(19) United States
(12) Patent Application Publication
(10) Pub. No.: US 2019/0169221 A1
(43) Pub. Date: Jun. 6, 2019

(54) SUBSTITUTED NUCLEOSIDES, NUCLEOTIDES AND ANALOGS THEREOF

(71) Applicant: Janssen BioPharma, Inc., South San Francisco, CA (US)

(72) Inventors: Guangyi WANG, Irvine, CA (US); Leonid BEIGELMAN, San Mateo, CA (US); Jerome DEVAL, El Granada, CA (US); Christian Andreas JEKLE, San Francisco, CA (US)

(21) Appl. No.: 16/324,862

(22) PCT Filed: Aug. 10, 2017
(86) PCT No.: PCT/US2017/046366

§ 371 Date: Feb. 11, 2019

Related U.S. Application Data
(60) Provisional application No. 62/374,537, filed on Aug. 12, 2016.

(51) Int. Cl. C07H 19/16; C07H 19/14; C07H 19/052; C07H 19/20; C07H 19/12; A61P 31/14

(52) U.S. Cl. CPC C07H 19/16; C07H 19/14; A61P 31/14; C07H 19/20; C07H 19/12; C07H 19/052

(57) ABSTRACT

Disclosed herein are nucleotide analogs, methods of synthesizing nucleotide analogs and methods of treating diseases and/or conditions such as a Picornaviridae and/or Flaviviridae viral infections with one or more nucleotide analogs.

### BACKGROUND

### Field

The present application relates to the fields of chemistry, biochemistry and medicine. More particularly, disclosed herein are nucleoside analogs, pharmaceutical compositions that include one or more nucleoside analogs and methods of synthesizing the same. Also disclosed herein are methods of treating viral diseases and/or conditions with a nucleotide analog, alone or in combination therapy with one or more other agents.

### Description

Nucleoside analogs are a class of compounds that have been shown to exert antiviral and anticancer activity both in vitro and in vivo, and thus, have been the subject of widespread research for the treatment of viral infections. Nucleoside analogs are usually therapeutically inactive compounds that are converted by host or viral enzymes to their respective active anti-metabolites, which, in turn, may inhibit polymerases involved in viral or cell proliferation. The activation occurs by a variety of mechanisms, such as the addition of one or more phosphate groups and, or in combination with, other metabolic processes.

### SUMMARY

Some embodiments disclosed herein relate to a compound of Formula (I), or a pharmaceutically acceptable salt thereof. Other embodiments disclosed herein relate to a compound of Formula (II), or a pharmaceutically acceptable salt thereof.

Some embodiments disclosed herein relate to a method of ameliorating and/or treating a Picornuviridae viral infection that can include administering to a subject identified as suffering from the Picornaviridae viral infection an effective amount of one or more compounds of Formulae (I) and/or (II), or a pharmaceutically acceptable salt of any of the foregoing, or a pharmaceutical composition that includes one or more compounds of Formulae (I) and/or (II), or a pharmaceutically acceptable salt of any of the foregoing. Other embodiments described herein relate to using one or more compounds of Formulae (I) and/or (II), or a pharmaceutically acceptable salt of any of the foregoing, in the manufacture of a medicament for ameliorating and/or treating a Picornaviridae viral infection. Still other embodiments described herein relate to one or more compounds of Formulae (I) and/or (II), or a pharmaceutically acceptable salt of any of the foregoing, or a pharmaceutical composition that includes one or more compounds of Formulae (I) and/or (II), or a pharmaceutically acceptable salt of any of the foregoing, that can be used for ameliorating and/or treating a Picornaviridae viral infection.

Some embodiments disclosed herein relate to a method of ameliorating and/or treating a Picornaviridae viral infection that can include contacting a cell infected with the picornavirus with an effective amount of one or more compounds described herein (for example, a compound of Formulae (I) and/or (II), or a pharmaceutically acceptable salt of any of the foregoing), or a pharmaceutical composition that includes one or more compounds described herein, or a pharmaceutically acceptable salt thereof. Other embodiments described herein relate to using one or more compounds described herein (for example, a compound of Formulae (I) and/or (II), or a pharmaceutically acceptable salt of any of the forgoing) in the manufacture of a medicament for ameliorating and/or treating a Picornaviridae viral infection that can include contacting a cell infected with the picornavirus with an effective amount of said compound(s), or a pharmaceutically acceptable salt thereof. Still other embodiments described herein relate to one or more compounds described herein (for example, a compound of Formulae (I) and/or (II), or a pharmaceutically acceptable salt of any of the foregoing), or a pharmaceutical composition that includes one or more compounds described herein, or a pharmaceutically acceptable salt thereof, that can be used for ameliorating and/or treating a Picornaviridae viral infection by contacting a cell infected with the picornavirus with an effective amount of said compound(s).

Some embodiments disclosed herein relate to a method of inhibiting replication of a Picornaviridae virus that can include contacting a cell infected with the picornavirus with an effective amount of one or more compounds described herein (for example, a compound of Formulae (I) and/or (II), or a pharmaceutically acceptable salt of any of the foregoing), or a pharmaceutical composition that includes one or more compounds described herein, or a pharmaceutically acceptable salt thereof. Other embodiments described herein relate to using one or more compounds described herein (for example, a compound of Formulae (I) and/or (II), or a pharmaceutically acceptable salt thereof) in the manufacture of a medicament for inhibiting replication of a Picornaviridae virus that can include contacting a cell infected with the Picornaviridae virus with an effective amount of said compound(s), or a pharmaceutically acceptable salt thereof. Still other embodiments described herein relate to one or more compounds described herein (for example, a compound of Formulae (I) and/or (II), or a pharmaceutically acceptable salt of any of the foregoing), or a pharmaceutical composition that includes one or more compounds described herein, or a pharmaceutically acceptable salt thereof, that can be used for inhibiting replication of a Picornaviridae virus by contacting a cell infected with the picornavirus with an effective amount of said compound(s), or a pharmaceutically acceptable salt thereof. In some embodiments, the Picornaviridae virus can be selected from a rhinovirus, hepatitis A virus, a coxasackie virus and an enterovirus.

Some embodiments disclosed herein relate to a method of ameliorating and/or treating a Flaviviridae viral infection that can include administering to a subject identified as suffering from the Flaviviridae viral infection an effective amount of one or more compounds of Formulae (I) and/or (II), or a pharmaceutically acceptable salt of any of the foregoing, or a pharmaceutical composition that includes one or more compounds of Formulae (I) and/or (II), or a pharmaceutically acceptable salt of any of the foregoing. Other embodiments disclosed herein relate to a method of ameliorating and/or treating a Flaviviridae viral infection that can include contacting a cell infected with the Flaviviridae virus with an effective amount of one or more compounds described herein (for example, a compound of Formulae (I) and/or (II), or a pharmaceutically acceptable salt of any of the foregoing), or a pharmaceutical composition that includes one or more compounds described herein, or a pharmaceutically acceptable salt thereof. Still other embodiments described herein relate to using one or more compounds of Formulae (I) and/or (II), or a pharmaceutically acceptable salt of any of the foregoing, in the manufacture of a medicament for ameliorating and/or treating a Flaviviridae viral infection. Yet still other embodiments described herein relate to one or more compounds of Formulae (I) and/or (II), or a pharmaceutically acceptable salt of any of the foregoing, or a pharmaceutical composition that includes one or more compounds of Formulae (I) and/or (II), or a pharmaceutically acceptable salt of any of the foregoing, that can be used for ameliorating and/or treating a Flaviviridae viral infection. Some embodiments disclosed herein relate to a method of inhibiting replication of a Flaviviridae virus that can include contacting a cell infected with the Flaviviridae with an effective amount of one or more compounds described herein (for example, a compound of Formulae (I) and/or (II), or a pharmaceutically acceptable salt of any of the foregoing), or a pharmaceutical composition that includes one or more compounds described herein, or a pharmaceutically acceptable salt thereof. Other embodiments described herein relate to using one or more compounds described herein (for example, a compound of Formulae (I) and/or (II), or a pharmaceutically acceptable salt thereof) in the manufacture of a medicament for inhibiting replication of a Flaviviridae virus. Still other embodiments described herein relate to one or more compounds described herein (for example, a compound of Formulae (I) and/or (II), or a pharmaceutically acceptable salt of any of the foregoing), or a pharmaceutical composition that includes one or more compounds described herein, or a pharmaceutically acceptable salt thereof, that can be used for inhibiting replication of a Flaviviridae virus. In some embodiments, the Flaviviridae virus can be selected from Hepatitis C (HCV), dengue and Zika.

### BRIEF DESCRIPTION OF THE DRAWINGS

FIG. 1 shows example HCV protease inhibitors.

FIG. 2 shows example nucleoside HCV polymerase inhibitors.

FIG. 3 shows example non-nucleoside HCV polymerase inhibitors.

FIG. 4 shows example NS5A inhibitors.

FIG. 5 shows example other antivirals.

FIG. 6 shows example compounds of Formula (CC) and alpha-thiotriphosphates thereof.

FIG. 7 shows example compounds of Formula (AA).

FIG. 8 shows example compounds of Formula (BB).

FIG. 9 shows example compounds of Formula (DD).

FIG. 10 shows example compounds of Formula (EE).

FIG. 11 shows example compounds of Formula (FF).

### DETAILED DESCRIPTION

The viruses within the Picornaviridae family are non-enveloped, positive sense, single-stranded, spherical RNA viruses with an icosahedral capsid. Picornavirus genomes are approximately 7-8 kilobases long and have an IRES (Internal Ribosomal Entry Site). These viruses are polyadenylated at the 3′ end, and have a VPg protein at the 5′ end in place of a cap. Genera within the Picornaviridae family include Aphthovirus, Aquamavirus, Avihepatovirus, Cardiovirus, Cosavirus, Dicipivirus, Enterovirus, Erbovirus, Hepatovirus, Kobuvirus, Megrivirus, Parechovirus, Rhinovirus, Salivirus, Sapelovirus, Senecavirus, Teschovirus and Tremovirus.

Enteroviruses are transmitted through the fecal-oral route and/or via aerosols of respiratory droplets, and are highly communicable. The genus of Enterovirus includes several species, including: enterovirus A, enterovirus B, enterovirus C, enterovirus D, enterovirus E, enterovirus F, enterovirus G, enterovirus H enterovirus J, rhinovirus A, rhinovirus B and rhinovirus C. Within a species of the aforementioned enteroviruses are the following serotypes: polioviruses, rhinoviruses, coxsackieviruses, echoviruses and enterovirus.

Rhinoviruses are the cause of the common cold. Rhinoviruses are named because of their transmission through the respiratory route and replication in the nose. A person can be infected with numerous Rhinoviruses over their lifetime because immunity develops for each serotype. Thus, each serotype can cause a new infection.

Hepatitis A is caused by infection with the hepatitis A virus, which is transmitted through the fecal-oral route. Person-to-person transmission can occur via ingestion of contaminated food or water, or through direct contact with an infectious individual.

Parechoviruses include human parechovirus 1 (echovirus 22), human parechovirus 2 (echovirus 23), human parechovirus 3, human parechovirus 4, human parechovirus 5 and human parechovirus 6.

Viruses in the Flaviviridae family are enveloped, positive sense, single-stranded, spherical RNA viruses with an icosahedral shaped capsid. These viruses are polyadenylated at the 5′ end but lack a 3′polyadenylate tail. Genera within the Flaviviridae family include: Flavivirus, Pestivirus and Hepacivirus. Flaviviridae viruses are predominantly arthropod-borne, and are often transmitted via mosquitos and ticks.

Hepaciviruses include Hepatitis C. Flaviviruses include several encephalitis viruses (for example, Japanese Encephalitis virus (JEV), St. Louis encephalitis virus (SLEV) and tick-borne encephalitis virus (TBEV), dengue virus 1-4 (DENV), West Nile virus (WNV), yellow fever virus (YFV), and Zika virus (ZIKV). Viruses within the Pestivirus genus include bovine viral diarrhea 1, bovine viral diarrhea 2 and classic swine fever virus.

### Definitions

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there are a plurality of definitions for a term herein, those in this section prevail unless stated otherwise.

As used herein, any “R” group(s) such as, without limitation, R^{A}, W^{1A}, R^{2A}, R^{3A}, R^{4A}, R^{5A}, R^{6A}, R^{7A}, R^{8A}, R^{9A}, R^{10A}, R^{11A}, R^{12A}, R^{13A}, R^{14A}, R^{15A}, R^{16A}, R^{17A}, R^{18A}, R^{19A}, R^{20A} and R^{21A} represent substituents that can be attached to the indicated atom. An R group may be substituted or unsubstituted. If two “R” groups are described as being “taken together” the R groups and the atoms they are attached to can form a cycloalkyl, cycloalkenyl, aryl, heteroaryl or heterocycle. For example, without limitation, if R^{a} and R^{b} of an NR^{a} R^{b} group are indicated to be “taken together,” it means that they are covalently bonded to one another to form a ring:

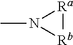

In addition, if two “R” groups are described as being “taken together” with the atom(s) to which they are attached to form a ring as an alternative, the R groups are not limited to the variables or substituents defined previously.

Whenever a group is described as being “optionally substituted” that group may be unsubstituted or substituted with one or more of the indicated substituents. Likewise, when a group is described as being “unsubstituted or substituted” if substituted, the substituent(s) may be selected from one or more of the indicated substituents. If no substituents are indicated, it is meant that the indicated “optionally substituted” or “substituted” group may be substituted with one or more group(s) individually and independently selected from alkyl, alkenyl, alkynyl, cycloalkyl, cycloalkenyl, aryl, heteroaryl, heterocyclyl, aryl(alkyl), heteroaryl(alkyl), (heterocyclyl)alkyl, hydroxy, alkoxy, acyl, cyano, halogen, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, an amino, a mono-substituted amine group and a di-substituted amine group.

As used herein, “Cₐ to C_{b}” in which “a” and “b” are integers refer to the number of carbon atoms in an alkyl, alkenyl or alkynyl group, or the number of carbon atoms in the ring of a cycloalkyl, cycloalkenyl, aryl, heteroaryl or heterocyclyl group. That is, the alkyl, alkenyl, alkynyl, ring of the cycloalkyl, ring of the cycloalkenyl, ring of the aryl, ring of the heteroaryl or ring of the heterocyclyl can contain from “a” to “b”, inclusive, carbon atoms. Thus, for example, a “C₁ to C₄ alkyl” group refers to all alkyl groups having from 1 to 4 carbons, that is, CH₃—, CH₃CH₂—, CH₃CH₂CH₂—, (CH₃)₂CH—, CH₃CH₂CH₂C₂—, CH₃CH₂CH(CH₃)— and (CH₃)₃C—. If no “a” and “b” are designated with regard to an alkyl, alkenyl, alkynyl, cycloalkyl cycloalkenyl, aryl, heteroaryl or heterocyclyl group, the broadest range described in these definitions is to be assumed.

As used herein, “alkyl” refers to a straight or branched hydrocarbon chain that comprises a fully saturated (no double or triple bonds) hydrocarbon group. The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 10 carbon atoms. The alkyl group could also be a lower alkyl having 1 to 6 carbon atoms. The alkyl group of the compounds may be designated as “C₁-C₄ alkyl” or similar designations. By way of example only, “C₁-C₄ alkyl” indicates that there are one to four carbon atoms in the alkyl chain, i.e., the alkyl chain is selected from methyl, ethyl, propyl, iso-propyl, n-butyl, iso-butyl, sec-butyl and t-butyl. Typical alkyl groups include, but are in no way limited to, methyl, ethyl, propyl, isopropyl, butyl, isobutyl, tertiary butyl, pentyl and hexyl. The alkyl group may be substituted or unsubstituted,

As used herein, “alkenyl” refers to an alkyl group that contains in the straight or branched hydrocarbon chain one or more double bonds. An alkenyl group may be unsubstituted or substituted.

As used herein, “alkynyl” refers to an alkyl group that contains in the straight or branched hydrocarbon chain one or more triple bonds. An alkynyl group may be unsubstituted or substituted.

As used herein, “cycloalkyl” refers to a completely saturated (no double or triple bonds) mono- or multi-cyclic hydrocarbon ring system. When composed of two or more rings, the rings may be joined together in a fused fashion. Cycloalkyl groups can contain 3 to 10 atoms in the ring(s) or 3 to 8 atoms in the ring(s). A cycloalkyl group may be unsubstituted or substituted. Typical cycloalkyl groups include, but are in no way limited to, cyclopropyl, cyclobutyl, cyclopentyl, cyclohexyl, cycloheptyl and cyclooctyl.

As used herein, “cycloalkenyl” refers to a mono- or multi-cyclic hydrocarbon ring system that contains one or more double bonds in at least one ring; although, if there is more than one, the double bonds cannot form a fully delocalized pi-electron system throughout all the rings (otherwise the group would be “aryl,” as defined herein). When composed of two or more rings, the rings may be connected together in a fused fashion. A cycloalkenyl can contain 3 to 10 atoms in the ring(s) or 3 to 8 atoms in the ring(s). A cycloalkenyl group may be unsubstituted or substituted.

As used herein, “aryl” refers to a carbocyclic (all carbon) monocyclic or multicyclic aromatic ring system (including fused ring systems where two carbocyclic rings share a chemical bond) that has a fully delocalized pi-electron system throughout all the rings. The number of carbon atoms in an aryl group can vary. For example, the aryl group can be a C₆-C₁₄ aryl group, a C₆-C₁₀ aryl group, or a C₆ aryl group. Examples of aryl groups include, but are not limited to, benzene, naphthalene and azulene. An aryl group may be substituted or unsubstituted.

As used herein, “heteroaryl” refers to a monocyclic, bicyclic and tricyclic aromatic ring system (a ring system with fully delocalized pi-electron system) that contain(s) one or more heteroatoms (for example, 1 to 5 heteroatoms), that is, an element other than carbon, including but not limited to, nitrogen, oxygen and sulfur. The number of atoms in the ring(s) of a heteroaryl group can vary. For example, the heteroaryl group can contain 4 to 14 atoms in the ring(s), 5 to 10 atoms in the ring(s) or 5 to 6 atoms in the ring(s). Furthermore, the term “heteroaryl” includes fused ring systems where two rings, such as at least one aryl ring and at least one heteroaryl ring, or at least two heteroaryl rings, share at least one chemical bond. Examples of heteroaryl rings include, but are not limited to, furan, furazan, thiophene, benzothiophene, phthalazine, pyrrole, oxazole, benzoxazole, 1,2,3-oxadiazole, 1,2,4-oxadiazole, thiazole, 1,2,3-thiadiazole, 1,2,4-thiadiazole, benzothiazole, imidazole, benzimidazole, indole, indazole, pyrazole, benzopyrazole, isoxazole, benzoisoxazole, isothiazole, triazole, benzotriazole, thiadiazole, tetrazole, pyridine, pyridazine, pyrimidine, pyrazine, purine, pteridine, quinoline, isoquinoline, quinazoline, quinoxaline, cinnoline and triazine. A heteroaryl group may be substituted or unsubstituted.

As used herein, “heterocyclyl” or “heteroalicyclyl” refers to three-, four-, five-, six-, seven-, eight-, nine-, ten-, up to 18-membered monocyclic, bicyclic and tricyclic ring system wherein carbon atoms together with from 1 to 5 heteroatoms constitute said ring system. For example, the heterocyclyl or heteroalicyclyl can contain 4 to 14 atoms in the ring(s), 5 to 10 atoms in the ring(s) or 5 to 6 atoms in the ring(s). A heterocycle may optionally contain one or more unsaturated bonds situated in such a way, however, that a fully delocalized pi-electron system does not occur throughout all the rings. The heteroatom(s) is an element other than carbon including, but not limited to, oxygen, sulfur and nitrogen. A heterocycle may further contain one or more carbonyl or thiocarbonyl functionalities, so as to make the definition include oxo-systems and thio-systems such as lactams, lactones, cyclic imides, cyclic thioimides and cyclic carbamates. When composed of two or more rings, the rings may be joined together in a fused fashion. Additionally, any nitrogens in a heteroalicyclyl may be quaternized. Heterocyclyl or heteroalicyclic groups may be unsubstituted or substituted. Examples of such “heterocyclyl” or “heteroalicyclyl” groups include but are not limited to, 1,3-dioxin, 1,3-dioxane, 1,4-dioxane, 1,2-dioxolane, 1,3-dioxolane, 1,4-dioxolane, 1,3-oxathiane, 1,4-oxathiin, 1,3-oxathiolane, 1,3-dithiole, 1,3-dithiolane, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, trioxane, hexahydro-1,3,5-triazine, imidazoline, imidazolidine, isoxazoline, isoxazolidine, oxazoline, oxazolidine, oxazolidinone, thiazoline, thiazolidine, morpholine, oxirane, piperidine N-Oxide, piperidine, piperazine, pyrrolidine, pyrrolidone, pyrrolidione, 4-piperidone, pyrazoline, pyrazolidine, 2-oxopyrrolidine, tetrahydropyran, 4H-pyran, tetrahydrothiopyran, thiamorpholine, thiamorpholine sulfoxide, thiamorpholine sulfone and their benzo-fused analogs (e.g., benzimidazolidinone, tetrahydroquinoline and 3,4-methylenedioxyphenyl).

As used herein, “aralkyl” and “aryl(alkyl)” refer to an aryl group connected, as a substituent, via a lower alkylene group. The lower alkylene and aryl group of an aryl(alkyl) may be substituted or unsubstituted. Examples include but are not limited to benzyl, 2-phenyl(alkyl), 3-phenyl(alkyl) and naphthyl(alkyl).

As used herein, “heteroaralkyl” and “heteroaryl(alkyl)” refer to a heteroaryl group connected, as a substituent, via a lower alkylene group. The lower alkylene and heteroaryl group of heteroaralkyl may be substituted or unsubstituted. Examples include but are not limited to 2-thienyl(alkyl), 3-thienyl(alkyl), furyl(alkyl), thienyl(alkyl), pyrrolyl(alkyl), pyridyl(alkyl), isoxazolyl(alkyl), imidazolyl(alkyl) and their benzo-fused analogs.

A “heteroalicyclyl(alkyl)” and “heterocyclyl(alkyl)” refer to a heterocyclic or a heteroalicyclylic group connected, as a substituent, via a lower alkylene group. The lower alkylene and heterocyclyl of a heteroalicyclyl(alkyl) may be substituted or unsubstituted. Examples include but are not limited tetrahydro-2H-pyran-4-yl(methyl), piperidin-4-yl(ethyl), piperidin-4-yl(propyl), tetrahydro-2H-thiopyran-4-yl(methyl) and 1,3-thiazinan-4-yl(methyl).

“Lower alkylene groups” are straight-chained —CH₂— tethering groups, forming bonds to connect molecular fragments via their terminal carbon atoms. Examples include but are not limited to methylene (—CH₂—), ethylene (—CH₂CH₂—), propylene (—CH₂CH₂CH₂—) and butylene (—CH₂CH₂CH₂CH₂—). A lower alkylene group can be substituted by replacing one or more hydrogen or deuterium of the lower alkylene group with a substituent(s) listed under the definition of “substituted.”

As used herein, “alkoxy” refers to the formula —OR wherein R is an alkyl, an alkenyl, an alkynyl, a cycloalkyl, a cycloalkenyl, aryl, heteroaryl, heterocyclyl, aryl(alkyl), (heteroaryl)alkyl or (heterocyclyl)alkyl is defined herein. A non-limiting list of alkoxys is methoxy, ethoxy, n-propoxy, 1-methylethoxy (isopropoxy), n-butoxy, iso-butoxy, sec-butoxy, tert-butoxy, phenoxy and benzoxy. An alkoxy may be substituted or unsubstituted.

As used herein, “acyl” refers to a hydrogen, deuterium, alkyl, alkenyl, alkynyl, or aryl connected, as substituents, via a carbonyl group. Examples include formyl, acetyl, propanoyl, benzoyl and acryl. An acyl may be substituted or unsubstituted.

As used herein, “hydroxyalkyl” refers to an alkyl group in which one or more of the hydrogen or deuterium atoms are replaced by a hydroxy group. Exemplary hydroxyalkyl groups include but are not limited to, 2-hydroxyethyl, 3-hydroxypropyl, 2-hydroxypropyl and 2,2-dihydroxyethyl. A hydroxyalkyl may be substituted or unsubstituted.

As used herein, “haloalkyl” refers to an alkyl group in which one or more of the hydrogen or deuterium atoms are replaced by a halogen (e.g., mono-haloalkyl, di-haloalkyl and tri-haloalkyl). Such groups include but are not limited to, chloromethyl, fluoromethyl, difluoromethyl, trifluoromethyl, 1-chloro-2-fluoromethyl and 2-fluoroisobutyl. A haloalkyl may be substituted or unsubstituted.

As used herein, “haloalkoxy” refers to an O-alkyl group in which one or more of the hydrogen or deuterium atoms are replaced by a halogen (e.g., mono-haloalkoxy, di-haloalkoxy and tri-haloalkoxy). Such groups include but are not limited to, chloromethoxy, fluoromethoxy, difluoromethoxy, trifluoromethoxy, 1-chloro-2-fluoromethoxy and 2-fluoroisobutoxy. A haloalkoxy may be substituted or unsubstituted.

A “sulfenyl” group refers to an “—SR” group in which R can be hydrogen, deuterium, alkyl, alkenyl, alkynyl, cycloalkyl, cycloalkenyl, aryl, heteroaryl, heterocyclyl, aryl(alkyl), (heteroaryl)alkyl or (heterocyclyl)alkyl. A sulfenyl may be substituted or unsubstituted.

A “sulfinyl” group refers to an “—S(═O)—R” group in which R can be the same as defined with respect to sulfenyl. A sulfinyl may be substituted or unsubstituted.

A “sulfonyl” group refers to an “SO₂R” group in which R can be the same as defined with respect to sulfenyl. A sulfonyl may be substituted or unsubstituted.

An “O-carboxy” group refers to a “RC(═O)O—” group in which R can be hydrogen, deuterium, alkyl, alkenyl, alkynyl, cycloalkyl, cycloalkenyl, aryl, heteroaryl, heterocyclyl, aryl(alkyl), (heteroaryl)alkyl or (heterocyclyl)alkyl, as defined herein. An O-carboxy may be substituted or unsubstituted.

The terms “ester” and “C-carboxy” refer to a “—C(═O)OR” group in which R can be the same as defined with respect to O-carboxy. An ester and C-carboxy may be substituted or unsubstituted.

A “thiocarbonyl” group refers to a “—C(═S)R” group in which R can be the same as defined with respect to O-carboxy. A thiocarbonyl may be substituted or unsubstituted.

A “trihalomethanesulfonyl” group refers to an “X₃CSO₂—” group wherein each X is a halogen.

A “trihalomethanesulfonamido” group refers to an “X₃CS(O)₂N(R_{A})—” group wherein each X is a halogen and R_{A} is hydrogen, deuterium, alkyl, alkenyl, alkynyl, cycloalkyl, cycloalkenyl, aryl, heteroaryl, heterocyclyl, aryl(alkyl), (heteroaryl)alkyl or (heterocyclyl)alkyl.

The term “amino” as used herein refers to a —NH₂ group.

The term “mono-substituted amine group” refers to an amino group where one hydrogen has been replaced with an R group, for example, “—NHR_{A},” in which R_{A} can be alkyl, alkenyl, alkynyl, cycloalkyl, cycloalkenyl, aryl, heteroaryl, heterocyclyl, aryl(alkyl), (heteroaryl)alkyl or (heterocyclyl)alkyl. The R_{A} can be substituted or unsubstituted.

The term “di-substituted amine group” refers to an amino group where both hydrogens have been replaced with R groups, for example, an “—NR_{A}R_{B}.” group in which R_{A} and R_{B} can be independently alkyl, alkenyl, alkynyl, cycloalkyl, cycloalkenyl, aryl, heteroaryl, heterocyclyl, aryl(alkyl), (heteroaryl)alkyl or (heterocyclyl)alkyl. R_{A} and R_{B} can independently be substituted or unsubstituted.

As used herein, the term “hydroxy” refers to a —OH group.

A “cyano” group refers to a “—CN” group.

The term “azido” as used herein refers to a —N₃ group.

An “isocyanato” group refers to a “—NCO” group.

A “thiocyanato” group refers to a “—CNS” group.

An “isothiocyanato” group refers to an “—NCS” group.

A “mercapto” group refers to an “—SH” group.

A “carbonyl” group refers to a C═O group.

An “S-sulfonamido” group refers to a “—SO₂N(R_{A}R_{B})” group in which R_{A} and R_{B} can be independently hydrogen, deuterium, alkyl, alkenyl, alkynyl, cycloalkyl, cycloalkenyl, aryl, heteroaryl, heterocyclyl, aryl(alkyl), (heteroaryl)alkyl or (heterocyclyl)alkyl. An S-sulfonamido may be substituted or unsubstituted.

An “N-sulfonamido” group refers to a “RSO₂N(R_{A})—” group in which R and R_{A} can be independently hydrogen, deuterium, alkyl, alkenyl, alkynyl, cycloalkyl, cycloalkenyl, aryl, heteroaryl, heterocyclyl, aryl(alkyl), (heteroaryl)alkyl or (heterocyclyl)alkyl. An N-sulfonamido may be substituted or unsubstituted.

An “O-carbamyl” group refers to a “—OC(═O)N(R_{A}R_{B})” group in which R_{A} and R_{B} can be independently hydrogen, deuterium, alkyl, alkenyl, alkynyl, cycloalkyl, cycloalkenyl, aryl, heteroaryl, heterocyclyl, aryl(alkyl), (heteroaryl)alkyl or (heterocyclyl)alkyl. An O-carbamyl may be substituted or unsubstituted.

An “N-carbamyl” group refers to an “ROC(═O)N(R_{A})—” group in which R and R_{A} can be independently hydrogen, deuterium, alkyl, alkenyl, alkynyl, cycloalkyl, cycloalkenyl, aryl, heteroaryl, heterocyclyl, aryl(alkyl), (heteroaryl)alkyl or (heterocyclyl)alkyl. An N-carbamyl may be substituted or unsubstituted.

An “O-thiocarbamyl” group refers to a “—OC(═S)—N(R_{A}R_{B})” group in which R_{A} and R_{B} can be independently hydrogen, deuterium, alkyl, alkenyl, alkynyl, cycloalkyl, cycloalkenyl, aryl, heteroaryl, heterocyclyl, aryl(alkyl), (heteroaryl)alkyl or (heterocyclyl)alkyl. An O-thiocarbamyl may be substituted or unsubstituted,

An “N-thiocarbamyl” group refers to an “ROC(═S)N(R_{A})—” group in which R and R_{A} can be independently hydrogen, deuterium, alkyl, alkenyl, alkynyl, cycloalkyl, cycloalkenyl, aryl, heteroaryl, heterocyclyl, aryl(alkyl), (heteroaryl)alkyl or (heterocyclyl)alkyl. An N-thiocarbamyl may be substituted or unsubstituted.

A “C-amido” group refers to a “—C(═O)N(R_{A}R_{B})” group in which R_{A} and R_{B} can be independently hydrogen, deuterium, alkyl, alkenyl, alkynyl, cycloalkyl, cycloalkenyl, aryl, heteroaryl, heterocyclyl, aryl(alkyl), (heteroaryl)alkyl or (heterocyclyl)alkyl. A C-amido may be substituted or unsubstituted.

An “N-amido” group refers to a “RC(═O)N(R_{A})—” group in which R and R_{A} can be independently hydrogen, deuterium, alkyl, alkenyl, alkynyl, cycloalkyl, cycloalkenyl, aryl, heteroaryl, heterocyclyl, aryl(alkyl), (heteroaryl)alkyl or (heterocyclyl)alkyl. An N-amido may be substituted or unsubstituted.

The term “halogen atom” or “halogen” as used herein, means any one of the radio-stable atoms of column 7 of the Periodic Table of the Elements, such as, fluorine, chlorine, bromine and iodine.

Where the numbers of substituents is not specified (e.g., haloalkyl), there may be one or more substituents present. For example “haloalkyl” may include one or more of the same or different halogens. As another example, “C₁-C₃ alkoxyphenyl” may include one or more of the same or different alkoxy groups containing one, two or three atoms.

As used herein, the abbreviations for any protective groups, amino acids and other compounds, are, unless indicated otherwise, in accord with their common usage, recognized abbreviations, or the IUPAC-IUB Commission on Biochemical Nomenclature (See, Biochem. 11:942-944 (1972)).

The term “nucleoside” is used herein in its ordinary sense as understood by those skilled in the art, and refers to a compound composed of an optionally substituted pentose moiety or modified pentose moiety attached to a heterocyclic base or tautomer thereof via a N-glycosidic bond, such as attached via the 9-position of a purine-base or the 1-position of a pyrimidine-base, or via a C-glycosidic bond, such as attached via the 7-position of an optionally substituted imidazo[2,1-f][1,2,4]triazine or an optionally substituted pyrrolo[2,1-f][1,2,4]triazine. Examples include, but are not limited to, a ribonucleoside comprising a ribose moiety and a deoxyribonucleoside comprising a deoxyribose moiety. A modified pentose moiety is a pentose moiety in which an oxygen atom has been replaced with a carbon and/or a carbon has been replaced with a sulfur or an oxygen atom. A “nucleoside” is a monomer that can have a substituted base and/or sugar moiety. Additionally, a nucleoside can be incorporated into larger DNA and/or RNA polymers and oligomers. In some instances, the nucleoside can be a nucleoside analog drug.

The term “nucleotide” is used herein in its ordinary sense as understood by those skilled in the art, and refers to a nucleoside having a phosphate ester bound to the pentose moiety, for example, at the 5′-position. A nucleotide may have one phosphate group (a “monophosphate”), two phosphate groups (a “diphosphate”) or three phosphate groups (a “triphosphate”).

As used herein, the term “heterocyclic base” refers to an optionally substituted nitrogen-containing heterocyclyl that can be attached to an optionally substituted pentose moiety or modified pentose moiety. In some embodiments, the heterocyclic base can be selected from an optionally substituted purine-base, an optionally substituted pyrimidine-base and an optionally substituted triazole-base (for example, a 1,2,4-triazole). The term “purine-base” is used herein in its ordinary sense as understood by those skilled in the art, and includes its mummers. Similarly, the term “pyrimidine-base” is used herein in its ordinary sense as understood by those skilled in the art, and includes its tautomers. A non-limiting list of optionally substituted purine-bases includes purine, adenine, guanine, hypoxanthine, xanthine, alloxanthine, 7-alkylguanine (e.g., 7-methylguanine), theobromine, caffeine, uric acid and isoguanine. Examples of pyrimidine-bases include, but are not limited to, cytosine, thymine, uracil, 5,6-dihydrouracil and 5-alkylcytosine 5-methylcytosine). An example of an optionally substituted triazole-base is 1,2,4-triazole-3-carboxamide. Other non-limiting examples of heterocyclic bases include diaminopurine, 8-oxo-N⁶-alkyladenine (e. 8-oxo-N⁶-methyladenine), 7-deazaxanthine, 7-deazaguanine, 7-deazaadenine, N⁴,N⁴-ethanocytosin, N⁶,N⁶-ethano-2,6-diaminopurine, 5-halouracil (e.g., 5-fluorouracil and 5-bromouracil), pseudoisocytosine, isocytosine, isoguanine, imidazo[2,1-f][1,2,4]triazine, pyrrolo[2,1-f][1,2,4]triazine, imidazo[2,1-f][1,2,4]triazine-4-amine, pyrrolo [2,1-f][1,2,4]triazine-4-amine and other heterocyclic bases described in U.S. Pat. Nos. 5,432,272 and 7,125,855, which are incorporated herein by reference for the limited purpose of disclosing additional heterocyclic bases. In some embodiments, a heterocyclic base can be optionally substituted with an amine or an enol protecting group(s).

The term “—N-linked amino acid” refers to an amino acid that is attached to the indicated moiety via a main-chain amino or mono-substituted amine group. When the amino acid is attached in an —N-linked amino acid, one of the hydrogen or deuteriums that is part of the main-chain amino or mono-substituted amine group is not present and the amino acid is attached via the nitrogen. N-linked amino acids can be substituted or unsubstituted.

The term “—N-linked amino acid ester derivative” refers to an amino acid in which a main-chain carboxylic acid group has been converted to an ester group. In some embodiments, the ester group has a formula selected from alkyl-O—C(═O)—, cycloalkyl-O—C(═O)—, aryl-O—C(═O)— and aryl(alkyl)-O—C(═O)—. A non-limiting list of ester groups include substituted and unsubstituted versions of the following: methyl-O—C(═O)—, ethyl-O—C(═O)—, n-propyl-O—C(═O)—, isopropyl-O—C(═O)—, n-butyl-O—C(═O)—, isobutyl-O—C(═O)—, tert-butyl-O—C(═O)—, neopentyl-O—C(═O)—, cyclopropyl-O—C(═O)—, cyclobutyl-O—C(═O)—, cyclopentyl-O—C(═O)—, cyclohexyl-O—C(═O)—, phenyl-O—C(═O)—, benzyl-O—C(═O)— and naphthyl-O—C(═O)—. N-linked amino acid ester derivatives can be substituted or unsubstituted.

The term “—O-linked amino acid” refers to an amino acid that is attached to the indicated moiety via the hydroxy from its main-chain carboxylic acid group. When the amino acid is attached in an —O-linked amino acid, the hydrogen or deuterium that is part of the hydroxy from its main-chain carboxylic acid group is not present and the amino acid is attached via the oxygen. O-linked amino acids can be substituted or unsubstituted.

As used herein, the term “amino acid” refers to any amino acid (both standard and non-standard amino acids), including, but not limited to, α-amino acids, β-amino acids, γ-amino acids and δ-amino acids. Examples of suitable amino acids include, but are not limited to, alanine, asparagine, aspartate, cysteine, glutamate, glutamine, glycine, proline, serine, tyrosine, arginine, histidine, isoleucine, leucine, lysine, methionine, phenylalanine, threonine, tryptophan and valine. Additional examples of suitable amino acids include, but are not limited to, ornithine, hypusine, 2-aminoisobutyric acid, dehydroalanine, gamma-aminobutyric acid, citrulline, beta-alanine, alpha-ethyl-glycine, alpha-propyl-glycine and norleucine.

The terms “phosphorothioate” and “phosphothioate” refer to a compound of the general formula

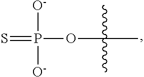

its protonated forms (for example,

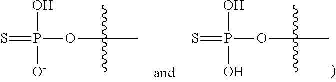 [SMILES: *OP([O-])(O)=S ; *OP(O)(O)=S]

and its tautomers (such as

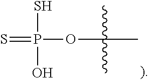 [SMILES: *OP(O)(=S)S]

As used herein, the term “phosphate” is used in its ordinary sense as understood by those skilled in the art, and includes its protonated forms (for example,

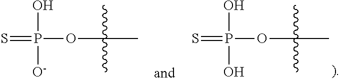 [SMILES: *OP([O-])(O)=S ; *OP(O)(O)=S]

As used herein, the terms “monophosphate,” “diphosphate,” and “triphosphate” are used in their ordinary sense as understood by those skilled in the art, and include protonated forms.

The terms “protecting group” and “protecting groups” as used herein refer to any atom or group of atoms that is added to a molecule in order to prevent existing groups in the molecule from undergoing unwanted chemical reactions. Examples of protecting group moieties are described in T. W. Greene and P. G. M. Wuts, Protective Groups in Organic Synthesis, 3. Ed. John Wiley & Sons. 1999 and in J. F. W. McOmie, Protective Groups in Organic Chemistry Plenum Press, 1973, both of which are hereby incorporated by reference for the limited purpose of disclosing suitable protecting groups. The protecting group moiety may be chosen in such a way, that they are stable to certain reaction conditions and readily removed at a convenient stage using methodology known from the art. A non-limiting list of protecting groups include benzyl; substituted benzyl; alkylcarbonyls and alkoxycarbonyls (e.g., t-butoxycarbonyl (BOC), acetyl, or isobutyryl); arylalkylcarbonyls and arylalkoxycarbonyls (e.g., benzyloxycarbonyl); substituted methyl ether (e.g., methoxymethyl ether); substituted ethyl ether; a substituted benzyl ether; tetrahydropyranyl ether; silyls (e.g., trimethylsilyl, triethylsilyl, triisopropylsilyl, t-butyldimethylsilyl, tri-iso-propylsilyloxymethyl, [2-(trimethylsilyl)ethoxy]methyl or t-butyldiphenylsilyl); esters (e.g., benzoate ester); carbonates (e.g., methoxymethylcarbonate); sulfonates (e.g., tosylate or mesylate); acyclic ketal (e.g., dimethyl acetal); cyclic ketals (e.g., 1,3-dioxane, 1,3-dioxolanes and those described herein); acyclic acetal; cyclic acetal (e.g., those described herein); acyclic hemiacetal; cyclic hemiacetal; cyclic dithioketals 1,3-dithiane or 1,3-dithiolane); orthoesters (e.g., those described herein) and triarylmethyl groups (e.g., trityl; monomethoxytrityl (MMTr); 4,4′-dimethoxytrityl (DMTr); 4,4′,4″-trimethoxytrityl (TMTr); and those described herein).

The term “pharmaceutically acceptable salt” refers to a salt of a compound that does not cause significant irritation to an organism to which it is administered and does not abrogate the biological activity and properties of the compound. In some embodiments, the salt is an acid addition salt of the compound. Pharmaceutical salts can be obtained by reacting a compound with inorganic acids such as hydrohalic acid (e.g., hydrochloric acid or hydrobromic acid), sulfuric acid, nitric acid and phosphoric acid. Pharmaceutical salts can also be obtained by reacting a compound with an organic acid such as aliphatic or aromatic carboxylic or sulfonic acids, for example formic, acetic, succinic, lactic, malic, tartaric, citric, ascorbic, nicotinic, methanesulfonic, ethanesulfonic, p-toluenesulfonic, salicylic or naphthalenesulfonic acid. Pharmaceutical salts can also be obtained by reacting a compound with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, C₁-C₇ alkylamine, cyclohexylamine, triethanolamine, ethylenediamine and salts with amino acids such as arginine and lysine.

Terms and phrases used in this application and variations thereof, especially in the appended claims, unless otherwise expressly stated, should be construed as open ended as opposed to limiting. As examples of any of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; and use of terms like ‘preferably,’ ‘preferred,’ ‘desired,’ or ‘desirable,’ and words of similar meaning should not be understood as implying that certain features are critical, essential, or even important to the structure or function, but instead as merely intended to highlight alternative or additional features that may or may not be utilized in a particular embodiment. In addition, the term “comprising” is to be interpreted synonymously with the phrases “having at least” or “including at least”. When used in the context of a process, the term “comprising” means that the process includes at least the recited steps, but may include additional steps. When used in the context of a compound, composition or device, the term “comprising” means that the compound, composition or device includes at least the recited features or components, but may also include additional features or components. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

It is understood that, in any compound described herein having one or more chiral centers, if an absolute stereochemistry is not expressly indicated, then each center may be independently of R-configuration or S-configuration or a mixture thereof. Thus, the compounds provided herein may be enantiomerically pure, enantiomerically enriched, racemic mixture, diastereomerically pure, diastereomerically enriched, or a stereoisomeric mixture. In addition it is understood that, in any compound described herein having one or more double bond(s) generating geometrical isomers that can be defined as E or Z, each double bond may be independently E or Z, or a mixture thereof.

Likewise, it is understood that, in any compound described, all tautomeric forms are also intended to be included. For example all tautomers of a phosphate and a phosphorothioate groups are intended to be included. Examples of tautomers of a phosphorothioate include the following:

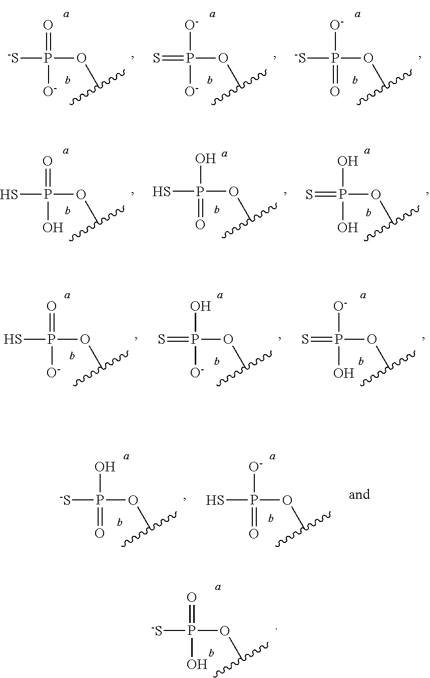

Examples of tautomers of a phosphate include the following:

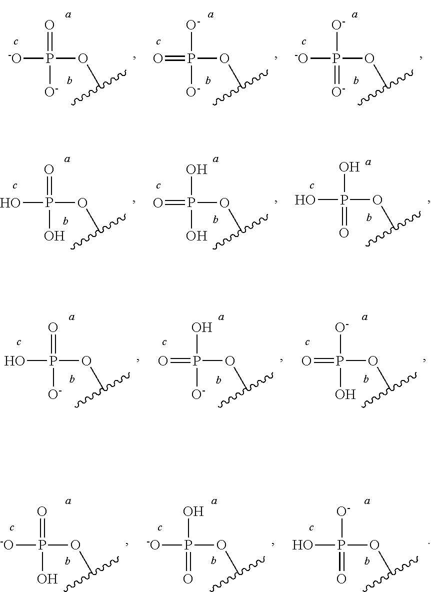

Furthermore, all tautomers of heterocyclic bases known in the art are intended to be included, including tautomers of natural and non-natural purine-bases and pyrimidine-bases.

It is to be understood that where compounds disclosed herein have unfilled valencies, then the valencies are to be filled as needed with hydrogen (also referred to as protium, hydrogen-1 or ¹H) or isotopes thereof. A suitable isotope of hydrogen is deuterium (also referred to as hydrogen-2 or ²H).

It is understood that the compounds described herein can be labeled isotopically. Substitution with isotopes such as deuterium may afford certain therapeutic advantages resulting from greater metabolic stability,such as, for example, increased in vivo half-life or reduced dosage requirements. Each chemical element as represented in a compound structure may include any isotope of said element. Thus, reference herein to a compound encompasses all potential isotopic forms unless the context clearly dictates otherwise or an isotope is already explicitly specified.

It is understood that the compounds, methods and combinations described herein include crystalline forms (also known as polymorphs, which include the different crystal packing arrangements of the same elemental composition of a compound), amorphous phases, salts, solvates and hydrates. In some embodiments, the compounds described herein (including those described in methods and combinations) exist in solvated forms with pharmaceutically acceptable solvents such as water, ethanol, or the like. In other embodiments, the compounds described herein (including those described in methods and combinations) exist in unsolvated form. Solvates contain either stoichiometric or non-stoichiometric amounts of a solvent, and may be formed during the process of crystallization with pharmaceutically acceptable solvents such as water, ethanol, or the like. Hydrates are formed when the solvent is water, or alcoholates are formed when the solvent is alcohol. In addition, the compounds provided herein can exist in unsolvated as well as solvated forms.

Where a range of values is provided, it is understood that the upper and lower limit, and each intervening value between the upper and lower limit of the range is encompassed within the embodiments.

### Compounds

Some embodiments disclosed herein relate to a compound of Formula (I), or a pharmaceutically acceptable salt thereof:

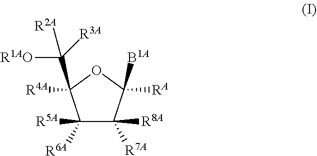

wherein can be

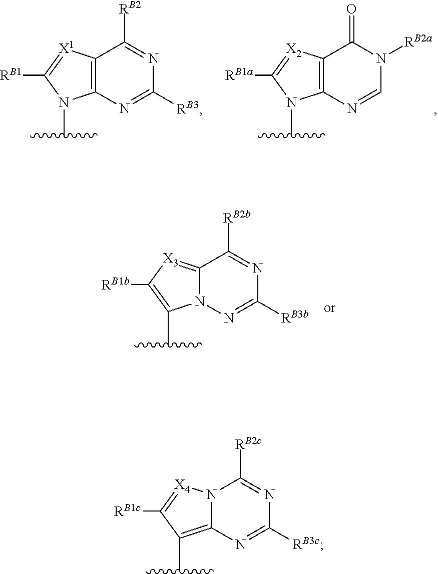

X¹ can be N (nitrogen) or —CR^{B6}; X² can be N (nitrogen) or —CR^{B6a}; X³ can be N (nitrogen) or —CR^{B6b}; X⁴ can be N (nitrogen) or —CR^{B6c}; R^{B1}, R^{B1a}, R^{B1b} and R^{B1c} can independently be selected from hydrogen or deuterium; R^{B2} can be NR^{B4a}R^{B4b}; R^{B2b} can be NR^{B4a1}R^{B4b1}; R^{B2c} can NR^{B4a2}R^{B4b2}; R^{B2a} can be selected from hydrogen, an optionally substituted C₁-C₆ alkyl, an optionally substituted C₂₋₆ alkenyl and an optionally substituted C₃₋₆ cycloalkyl; R^{B3} can be hydrogen, deuterium, halogen or NR^{B5a}R^{B5b}, R^{B3b} can be hydrogen, deuterium, halogen or NR^{B5a1}R^{B5b1}; R^{B3c} can be hydrogen, deuterium, halogen or NR^{B5a2}R^{B5b2}; R^{B4a}, R^{B4a1} and R^{B4a2} can be independently hydrogen or deuterium; R^{B4b}, R^{B4b1} and R^{B4b2} can be independently selected from hydrogen, deuterium, an optionally substituted C₁₋₆ alkyl, an optionally substituted C₂₋₆ alkenyl, an optionally substituted C₃₋₆ cycloalkyl, —C(═O)R^{B7} and —C(═O)OR^{B8}; R^{B5a} can be hydrogen or deuterium; R^{B5b} can be selected from hydrogen, an optionally substituted C₁₋₆ alkyl, an optionally substituted C₂₋₆ alkenyl, an optionally substituted C₃₋₆ cycloalkyl, —C(═O)R^{B9} and —C(═O)OR^{B10}; R^{B6}, R^{B6a}, R^{B6b} and R^{B6c} can independently be selected from hydrogen, deuterium, halogen, —C≡N, —C(═O)NH₂, an optionally substituted C₁₋₆ alkyl, an optionally substituted C₂₋₆ alkenyl and an optionally substituted C₂₋₆ alkynyl; R^{B7}, R^{B8}, R^{B9} and R^{B10} can independently be selected from an optionally substituted C₁₋₆ alkyl, an optionally substituted C₂₋₆ alkenyl, an optionally substituted C₂₋₆ alkynyl, an optionally substituted C₃₋₆ cycloalkyl, an optionally substituted C₅₋₁₀ cycloalkenyl, an optionally substituted C₆₋₁₀ aryl, an optionally substituted heteroaryl, an optionally substituted heterocyclyl, an optionally substituted aryl (C₁₋₆ alkyl), an optionally substituted heteroaryl (C₁₋₆ alkyl) and an optionally substituted heterocyclyl (C₁₋₆ alkyl); R^{1A} can be hydrogen, an optionally substituted acyl, an optionally substituted O-linked amino acid or

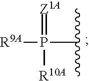

R^{2A}, R^{3A}, R^{5A} and R^{A} can independently be hydrogen or deuterium; R^{4A} can be hydrogen, deuterium or fluoro; R^{6A} can be selected from —OH, —OC(═O)R″^{A} and an optionally substituted O-linked amino acid; R^{7A} can be —OH, —OC(═O)R″^{B}, fluoro or chloro; R^{8A} can be an optionally substituted C₁₋₃ alkyl, an optionally substituted C₂₋₆ alkenyl or an optionally substituted C₂₋₆ alkynyl; R^{9A} and R^{10A} can independently be selected from of O⁻, —OH, an optionally substituted O—C₁₋₂₄ alkyl, an optionally substituted —O—C₂₋₂₄ alkenyl, an optionally substituted —O—C₂₋₂₄ alkenyl, an optionally substituted —O—C₃₋₆ cycloalkyl, an optionally substituted —O—C₅₋₁₀ cycloalkenyl, an optionally substituted —O-aryl, an optionally substituted —O-heteroaryl, an optionally substituted —O-aryl(C₁₋₆ alkyl), an optionally substituted *—O—(CR^{11A}R^{12A})ₚ—O—C₁₋₂₄ alkyl, an optionally substituted *—O—(CR^{13A}R^{14A})_{q}—O—C₂₋₂₄ alkenyl,

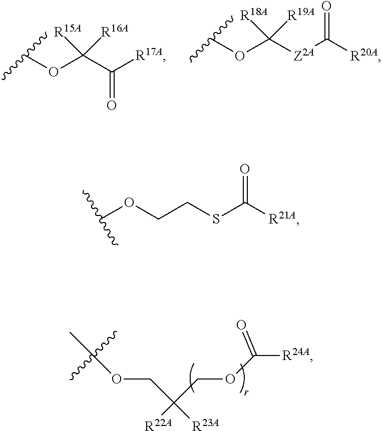

an optionally substituted N-linked amino acid and an optionally substituted N-linked amino acid ester derivative; or R^{9A} can be

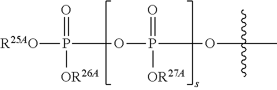

and R^{10A} can be O⁻ or OH; or R^{9A} and R^{10A} can be taken together to form a moiety selected from an optionally substituted

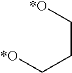

and an optionally substituted

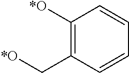

(wherein the asterisks indicate the points of attachment of the moieties), wherein the phosphorus and the moiety form a six-membered to ten-membered ring system; each R^{11A}, each R^{12A}, each R^{13A} and each R^{14A} can be independently hydrogen, deuterium, an optionally substituted C₁₋₂₄ alkyl or alkoxy; R^{15A}, R^{16A}, R^{18A} and R^{19A} can be independently selected from hydrogen, deuterium, an optionally substituted C₁₋₂₄ alkyl and an optionally substituted aryl; R^{17A} and R^{20A} can be independently selected from hydrogen, deuterium, an optionally substituted C₁₋₂₄ alkyl, an optionally substituted aryl, an optionally substituted —O—C₁₋₂₄ alkyl, an optionally substituted —O-aryl, an optionally substituted —O-heteroaryl and an optionally substituted —O-monocyclic heterocyclyl; R^{21A} can be selected from hydrogen, deuterium, an optionally substituted C₁₋₂₄ alkyl and an optionally substituted aryl; R^{22A} and R^{23A} can be independently selected from —C≡N, an optionally substituted C₂₋₈ organylcarbonyl, an optionally substituted C₂₋₈ alkoxycarbonyl and an optionally substituted C₂₋₈ organylaminocarbonyl; R^{24A} can be selected from hydrogen, deuterium, an optionally substituted C₁₋₂₄-alkyl, an optionally substituted C₂₋₂₄ alkenyl, an optionally substituted C₂₋₂₄ alkynyl, an optionally substituted C₃₋₆ cycloalkyl and an optionally substituted C₅₋₁₀ cycloalkenyl; R^{25A}, R^{26A} and R^{27A} can be independently absent, hydrogen or deuterium; p and q can be independently selected from 1, 2 and 3; r can be 1 or 2; s can be 0 or 1; R″^{A} and R″^{B} can be independently an optionally substituted C₁₋₂₄ alkyl; and Z^{1A} and Z^{2A} can be independently oxygen (O) or sulfur (S).

In some embodiments, R^{1A} can be hydrogen or deuterium. In some embodiments, R^{1A} can be an optionally substituted acyl. In other embodiments, R^{1A} can be —C(═O)R″^{A1}, wherein R″^{A1} can be an optionally substituted C₁₋₁₂ alkyl. In some embodiments, R″^{A1} can be an unsubstituted C₁₋₄ alkyl.

In still other embodiments, R^{1A} can be an optionally substituted O-linked amino acid, such as an optionally substituted O-linked a-amino acid. In some embodiments, R^{1A} can be an unsubstituted O-linked α-amino acid. Examples of suitable O-linked amino acids include alanine, asparagine, aspartate, cysteine, glutamate, glutamine, glycine, proline, serine, tyrosine, arginine, histidine, isoleucine, leucine, lysine, methionine, phenylalanine, threonine, tryptophan and valine. Additional examples of suitable amino acids include, but are not limited to, ornithine, hypusine, 2-aminoisobutyric acid, dehydroalanine, gamma-aminobutyric acid, citrulline, beta-alanine, alpha-ethyl-glycine, alpha-propyl-glycine and norleucine. In some embodiments, the O-linked amino acid can have the structure

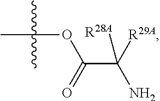

wherein R^{28A} can be selected from hydrogen, deuterium, an optionally substituted C₁₋₆ alkyl, an optionally substituted C₁₋₆ haloalkyl, an optionally substituted C₃₋₆ cycloalkyl, an optionally substituted C₆ aryl, an optionally substituted C₁₀ aryl and an optionally substituted aryl (C₁₋₆ alkyl); and R^{29A} can be hydrogen, deuterium or an optionally substituted C₁₋₄-alkyl; or R^{28A} and R^{29A} can be taken together to form an optionally substituted C₃₋₆ cycloalkyl. Those skilled in the art understand that when R^{1A} is an optionally substituted O-linked amino acid, the oxygen of R^{1A}O— of Formula (I) is part of the optionally substituted O-linked amino acid. For example, when R^{1A} is

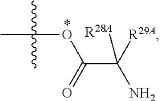

the oxygen indicated with “*” is the oxygen of R^{1A}O— of Formula (I).

When R^{28A} is substituted, R^{28A} can be substituted with one or more substituents selected from N-amido, mercapto, alkylthio, an optionally substituted aryl, hydroxy, an optionally substituted heteroaryl, O-carboxy and amino. In some embodiments, R^{28A} can be an unsubstituted. C₁₋₆-alkyl, such as those described herein. In some embodiments, R^{28A} can be hydrogen or deuterium. In other embodiments, R^{28A} can be methyl. In some embodiments, R^{29A} can be hydrogen or deuterium. In other embodiments, R^{29A} can be an optionally substituted C₁₋₄-alkyl, such as methyl, ethyl, n-propyl, isopropyl, n-butyl, isobutyl and tert-butyl. In an embodiment, R^{29A} can be methyl. Depending on the groups that are selected for R^{28A} and R^{29A}, the carbon to which R^{28A} and R^{29A} are attached may be a chiral center. In some embodiment, the carbon to which R^{28A} and R^{29A} are attached may be a (R)-chiral center. In other embodiments, the carbon to which R^{28A} and R^{29A} are attached may be a (S)-chiral center.

Examples of suitable

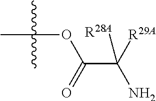

include the following:

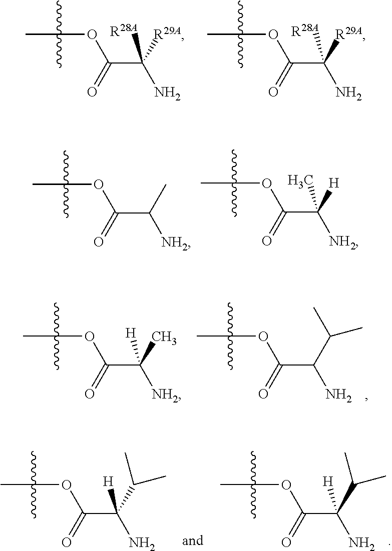

In some embodiments, R^{1A} can be

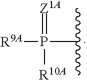

A variety of R^{9A} and R^{10A} groups can be attached to the phosphorus atom of Formula (I). In some embodiments, R^{9A} and R^{10A} can be both —OH. In other embodiments, R^{9A} and R^{10A} can be both O⁻. In still other embodiments, at least one R^{9A} and R^{10A} can be absent. In yet still other embodiments, at least one R^{9A} and R^{10A} can be hydrogen or deuterium. Those skilled in the art understand that when R^{9A} and/or R^{10A} are absent, the associated oxygen(s) will have a negative charge. For example, when R^{9A} is absent, the oxygen associated with R^{9A} will have a negative charge. In some embodiments, Z^{1A} can be O (oxygen). In other embodiments, Z^{1A} can be S (sulfur). In some embodiments, R^{1A} can be a monophosphate. In other embodiments, R^{1A} can be a monothiophosphate.

In some embodiments, one of R^{9A} and R^{10A} can be O⁻ or —OH and the other of R^{9A} and R^{10A} can be selected from an optionally substituted —O—C₁₋₂₄ alkyl, an optionally substituted —O—C₂₋₂₄ alkenyl, an optionally substituted —O—C₂₋₂₄ alkynyl, an optionally substituted —O—C₃₋₆ cycloalkyl, an optionally substituted —O—C₅₋₁₀ cycloalkenyl, an optionally substituted —O-aryl, an optionally substituted —O-heteroaryl and an optionally substituted —O-aryl (C₁₋₆ alkyl). In some embodiments, one of R^{9A} and R^{10A} can be O⁻ or —OH and the other of R^{9A} and R^{10A} can be an optionally substituted —O—C₁₋₂₄ alkyl. In other embodiments, both R^{9A} and R^{10A} can be independently selected from an optionally substituted —O—C₁₋₂₄ alkyl, an optionally substituted —O—C₂₋₂₄ alkenyl, an optionally substituted —O—C₂₋₂₄ alkynyl, an optionally substituted —O—C₃₋₆ cycloalkyl, an optionally substituted —O—C₅₋₁₀ cycloalkenyl, an optionally substituted —O-aryl, an optionally substituted —O-heteroaryl and an optionally substituted —O-aryl (C₁₋₆ alkyl). In some embodiments, both R^{9A} and R^{10A} can be an optionally substituted —O—C₁₋₂₄ alkyl. In other embodiments, both R^{9A} and R^{10A} can be an optionally substituted —O—C₂₋₂₄ alkenyl. In some embodiments, R^{9A} and R^{10A} can be independently an optionally substituted group selected from the following: —O-myristoleyl, —O-myristyl, —O-palmitoleyl, —O-palmityl, —O-sapienyl, —O-oleyl, —O-elaidyl, —O-vaccenyl, —O-linoleyl, —O-α-linolenyl, —O-arachidonyl, —O-eicosapentaenyl, —O-erucyl, —O-docosahexaenyl, —O-capryl, —O-lauryl, —O-stearyl, —O-arachidyl, —O-behenyl, —O-lignoceryl and —O-cerotyl.

In some embodiments, at least one of R^{9A} and R^{10A} can be an optionally substituted *—O—(CR^{11A}R^{12A})ₚ—O—C₁₋₂₄ alkyl. In other embodiments, R^{9A} and R^{10A} can be both an optionally substituted *—O—(CR^{11A}R^{12A})ₚ—O—C₁₋₂₄ alkyl. In some embodiments, each R^{11A} and each R^{12A} can be hydrogen or deuterium. In other embodiments, at least one of R^{11A} and R^{12A} can be an optionally substituted C₁₋₂₄ alkyl. In other embodiments, at least one of R^{11A} and R^{12A} can be an alkoxy (for example, benzoxy). In some embodiments, p can be 1. In other embodiments, p can be 2. In still other embodiments, p can be 3.

In some embodiments, at least one of R^{9A} and R^{10A} can be an optionally substituted *—O—(CR¹³R^{14A})_{q}—O—C₁₋₂₄ alkenyl. In other embodiments, R^{9A} and R^{10A} can be both an optionally substituted *—O—(CR^{13A}R^{14A})_{q}—O—C₁₋₂₄ alkenyl. In some embodiments, each R^{13A} and each R^{14A} can be hydrogen or deuterium. In other embodiments, at least one of R^{13A} and R^{14A} can be an optionally substituted C₁₋₂₄ alkyl. In some embodiments, q can be 1. In other embodiments, q can be 2. In still other embodiments, q can be 3. When at least one of R^{9A} and R^{10A} is *—O—(CR^{11A}R^{12A})ₚ—O—C₁₋₂₄ alkyl or an optionally substituted *—O—(CR^{13A}R^{14A})_{q}—O—C₁₋₂₄ alkenyl, the C₁₋₂₄ alkyl can be selected from caprylyl, capryl, lauryl, myristyl, palmityl, stearyl, arachidyl, behenyl, lignoceryl and cerotyl, and the C₂₋₂₄ alkenyl can be selected from myristoleyl, palmitoleyl, sapienyl, oleyl, elaidyl, vaccenyl, linoleyl, α-linolenyl, arachidonyl, eicosapentaenyl, erucyl and docosahexaenyl.

In some embodiments, at least one of R^{9A} and R^{10A} can be selected from

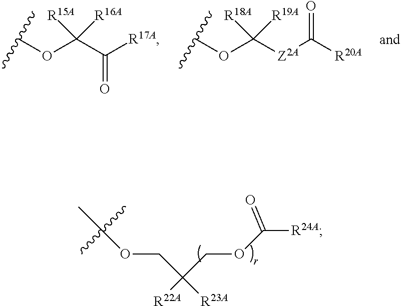

and the other of R^{9A} and R^{10A} can be selected from O⁻, —OH, an optionally substituted —O—C₁₋₂₄ alkyl, an optionally substituted —O—C₂₋₂₄ alkenyl, an optionally substituted —O—C₂₋₂₄ alkynyl, an optionally substituted —O—C₃₋₆ cycloalkyl, an optionally substituted —O—C₅₋₁₀ cycloalkenyl, an optionally substituted —O-aryl, an optionally substituted —O-heteroaryl and an optionally substituted —O-aryl (C₁₋₆ alkyl).

In some embodiments, at least one of R^{9A} and R^{10A} can be

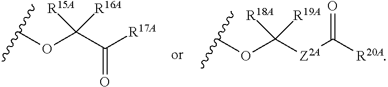

In some embodiments, both R^{9A} and R^{10A} can be

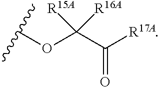

When one or both of R^{9A} and R^{10A} are

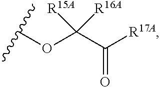

R^{15A} and R^{16A} can be independently selected from hydrogen, deuterium, an optionally substituted C₁₋₂₄ alkyl and an optionally substituted aryl; and R^{17A} can be selected from hydrogen, deuterium, an optionally substituted C₁₋₂₄ alkyl, an optionally substituted aryl, an optionally substituted —O—C₁₋₂₄ alkyl, an optionally substituted —O-aryl, an optionally substituted —O-heteroaryl and an optionally substituted —O-monocyclic heterocyclyl. In some embodiments, R^{15A} and R^{16A} can be hydrogen or deuterium. In other embodiments, at least one of R^{15A} and R^{16A} can be an optionally substituted C₁₋₂₄ alkyl or an optionally substituted aryl. In some embodiments, R^{17A} can be an optionally substituted C₁₋₂₄ alkyl. In some embodiments, R^{17A} can be an unsubstituted C₁₄ alkyl. In other embodiments, R^{17A} can be an optionally substituted aryl. In still other embodiments, R^{17A} can be an optionally substituted —O—C₁₋₂₄ alkyl, an optionally substituted —O-aryl, an optionally substituted —O-heteroaryl or an optionally substituted —O-monocyclic heterocyclyl. In some embodiments, R^{17A} can be an unsubstituted alkyl.

In some embodiments, both R^{9A} and R^{10A} can be

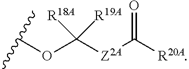

When one or both of R^{9A} and R^{10A} are

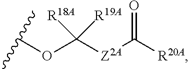

R^{18A} and R^{19A} can be independently selected from hydrogen, deuterium, an optionally substituted C₁₋₂₄ alkyl and an optionally substituted aryl; R^{20A} can be independently selected from hydrogen, deuterium, an optionally substituted C₁₋₂₄ alkyl, an optionally substituted aryl, an optionally substituted —O—C₁₋₂₄ alkyl, an optionally substituted —O-aryl, an optionally substituted —O-heteroaryl and an optionally substituted —O-monocyclic heterocyclyl; and Z^{2A} can be independently O (oxygen) or S (sulfur). In some embodiments, R^{18A} and R^{19A} can be hydrogen or deuterium. In other embodiments, at least one of R^{18A} and R^{19A} can be an optionally substituted C₁₋₂₄ alkyl or an optionally substituted aryl. In some embodiments, R^{20A} can be an optionally substituted C₁₋₂₄ alkyl. In some embodiments, R^{20A} can be an unsubstituted C₁₋₄ alkyl. In other embodiments, R^{20A} can be an optionally substituted aryl. In still other embodiments, R^{20A} can be an optionally substituted —O—C₁₋₂₄ alkyl, an optionally substituted —O-aryl, an optionally substituted —O-heteroaryl or an optionally substituted —O-monocyclic heterocyclyl. In some embodiments, R^{20A} can be an unsubstituted —O—C₁₋₄ alkyl. In some embodiments, Z^{2A} can be O (oxygen). In other embodiments, Z^{2A} can be or S (sulfur). In some embodiments, one or both of R^{9A} and R^{10A} can be an optionally substituted isopropyloxycarbonyloxymethoxy (POC). In some embodiments, R^{9A} and R^{10A} each can be an optionally substituted isopropyloxycarbonyloxymethoxy (POC) group, and form an optionally substituted bis(isopropyloxycarbonyloxymethyl) (bis(POC)) prodrug. In other embodiments, one or both of R^{9A} and R^{10A} can be an optionally substituted pivaloyloxymethoxy (POM). In some embodiments, R^{9A} and R^{10A} each can be an optionally substituted pivaloyloxymethoxy (POM) group, and form an optionally substituted bis(pivaloyloxymethyl) (bis(POM)) prodrug.

In some embodiments, at least one of R^{9A} and R^{10A} can be

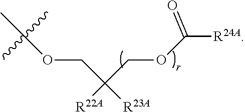

In some embodiments, both R^{9A} and R^{10A} can be

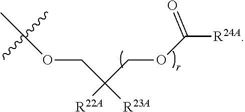

When one or both of R^{9A} and R^{10A} are

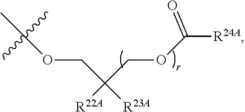

R^{22A} and R^{23A} can be independently —C≡N or an optionally substituted substituent selected from C₂₋₈ organylcarbonyl, C₂₋₈ alkoxycarbonyl and C₂₋₈ organylaminocarbonyl; R^{24A} can be selected from hydrogen, deuterium, an optionally substituted C₁₋₂₄ alkyl, an optionally substituted C₂₋₂₄ alkenyl, an optionally substituted C₂₋₂₄ alkynyl, an optionally substituted C₃₋₆ cycloalkyl and an optionally substituted C₅₋₁₀ cycloalkenyl; and r can be 1 or 2. In some embodiments, R^{22A} can be —C═N and R^{23A} can be an optionally substituted C₂₋₈ alkoxycarbonyl, such as —C(═O)OCH₃. In other embodiments, R^{22A} can be —C≡N and R^{23A} can be an optionally substituted C₂₋₈ organylaminocarbonyl, for example, C(═O)NHCH₂CH₃ and —C(═O)NHCH₂CH₂phenyl. In some embodiments, both R^{22A} and R^{23A} can be an optionally substituted C₂₋₈ organylcarbonyl, such as —C(═O)CH₃. In some embodiments, both R^{22A} and R^{23A} can be an optionally substituted. C₁₋₈ alkoxycarbonyl, for example, —C(═O)OCH₂CH₃ and —C(═O)OCH₃. In some embodiments, including those described in this paragraph, R^{24A} can be an optionally substituted C₁₋₄ alkyl. In some embodiment, R^{24A} can be methyl or tert-butyl. In some embodiments, r can be 1. In other embodiments, r can be 2.

In some embodiments, R^{9A} and R^{10A} can be both an optionally substituted —O-aryl. In some embodiments, at least one of R^{9A} and R^{10A} can be an optionally substituted —O-aryl. For example, both R^{9A} and R^{10A} can be an optionally substituted —O-phenyl or an optionally substituted —O-naphthyl. When substituted, the substituted —O-aryl can be substituted with 1, 2, 3 or more than 3 substituents. When more than two substituents are present, the substituents can be the same or different. In some embodiments, when at least one of R^{9A} and R^{10A} is a substituted —O-phenyl, the substituted —O-phenyl can be a para, ortho- or meta-substituted.

In some embodiments, R^{9A} and R^{10A} can be both an optionally substituted —O-aryl (C₁₋₆ alkyl). In some embodiments, at least one of R^{9A} and R^{10A} can be an optionally substituted —O-aryl (C₁₋₆ alkyl). For example, both R^{9A} and R^{10A} can be an optionally substituted —O-benzyl. When substituted, the substituted —O-benzyl group can be substituted with 1, 2, 3 or more than 3 substituents. When more than two substituents are present, the substituents can be the same or different. In some embodiments, the —O-aryl group of the aryl (C₁₋₆ alkyl) can be a para-, ortho- or meta-substituted phenyl.

In some embodiments, at least one of R^{9A} and R^{10A} can be

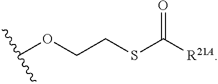

In some embodiments, R^{9A} and R^{10A} can be both

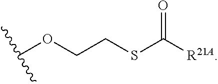

In some embodiments, at least one of R^{9A} and R^{10A} can be

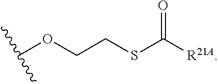

In some embodiments, R^{21A} can be hydrogen or deuterium. In other embodiments, R^{21A} can be an optionally substituted C₁₋₂₄ alkyl. In still other embodiments, R^{21A} can be an optionally substituted aryl (for example, an optionally substituted phenyl). In some embodiments, R^{21A} can be a C₁₋₆ alkyl, for example, methyl, ethyl, n-propyl, isopropyl, n-butyl, isobutyl, tut-butyl, pentyl (branched and straight-chained) and hexyl (branched and straight-chained). In some embodiments, R^{9A} and R^{10A} can be both an optionally substituted S-acylthioethoxy (SATE) group and form an optionally substituted SATE ester prodrug.

In some embodiments, R^{9A} and R^{10A} can be taken together to form an optionally substituted

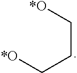

For example, when R^{9A} and R^{10A} can be taken together, the resulting moiety can be an optionally substituted

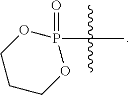 [SMILES: *P1(=O)OCCCO1]

When substituted, the ring can be substituted 1, 2, 3 or 3 or more times. When substituted with multiple substituents, the substituents can be the same or different. In some embodiments, the ring

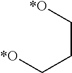

can be substituted with an optionally substituted aryl group and/or an optionally substituted heteroaryl. An example of a suitable heteroaryl is pyridinyl. In some embodiments, R^{5A} and R^{6A} can be taken together to form an optionally substituted

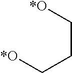

such as

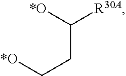

wherein R^{30A} can be an optionally substituted aryl, an optionally substituted heteroaryl or an optionally substituted heterocyclyl. In this paragraph, the asterisks indicate the points of attachment of the moieties. In some embodiments, R^{9A} and R^{10A} can form an optionally substituted cyclic 1-aryl-1,3-propanyl ester (HepDirect) prodrug moiety.

In some embodiments, R^{9A} and R^{10A} can be taken together to form an optionally substituted

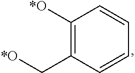

wherein the phosphorus and the moiety form a six-membered to ten-membered ring system. Example of an optionally substituted

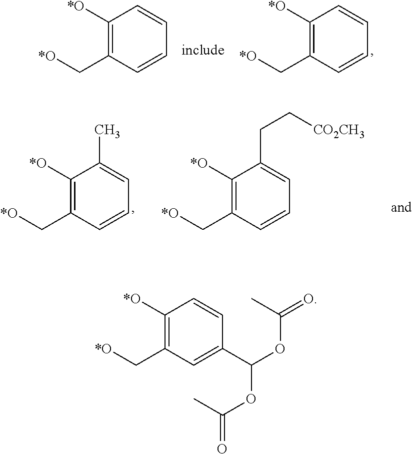

In this paragraph, the asterisks indicate the points of attachment of the moieties. In some embodiments, R^{9A} and R^{10A} can form an optionally substituted cyclosaligenyl (cycloSal) prodrug.

In other embodiments, R^{9A} can be an optionally substituted —O-aryl; and R^{10A} can be an optionally substituted N-linked amino acid or an optionally substituted N-linked amino acid ester derivative. In still other embodiments, R^{9A} can be an optionally substituted —O-heteroaryl; and R^{10A} can be an optionally substituted N-linked amino acid or an optionally substituted N-linked amino acid ester derivative.

In some embodiments, when R^{9A} can be an optionally substituted —O-aryl, R^{9A} can be an optionally substituted —O-phenyl. When the phenyl is substituted, the ring can be substituted 1, 2, 3 or more than 3 times. When substituted, the phenyl can be substituted at one or both ortho positions, one or both meta positions and/or the para position. In some embodiments, R^{9A} can be an unsubstituted —O-aryl. In some embodiments, R^{9A} can be an optionally substituted —O-naphthyl. In some embodiments, R^{9A} can be an unsubstituted —O-phenyl. In some embodiments, R^{9A} can be an unsubstituted —O-naphthyl.

In some embodiments, when R^{10A} can be an optionally substituted N-linked amino acid or an optionally substituted N-linked amino acid ester derivative, such as an optionally substituted N-linked α-amino acid or an optionally substituted N-linked α-amino acid ester derivative. Various amino acids are suitable, including those described herein. Examples of suitable amino acids include, but are not limited to, alanine, asparagine, aspartate, cysteine, glutamate, glutamine, glycine, proline, serine, tyrosine, arginine, histidine, isoleucine, leucine, lysine, methionine, phenylalanine, threonine, tryptophan and valine. In other embodiments, R^{10A} can be an optionally substituted N-linked amino acid ester derivative. Examples of suitable amino acid ester derivatives include, but are not limited to, an ester derivative of any of the following amino acids, alanine, asparagine, aspartate, cysteine, glutamate, glutamine, glycine, proline, serine, tyrosine, arginine, histidine, isoleucine, leucine, lysine, methionine, phenylalanine, threonine, tryptophan and valine. Additional examples of N-linked amino acid ester derivatives include, but are not limited to, an ester derivative of any of the following amino acids: alpha-ethyl-glycine, alpha-propyl-glycine and beta-alanine. In some embodiments, the N-linked amino acid ester derivative can be selected from N-alanine isopropyl ester, N-alanine cyclohexyl ester, N-alanine neopentyl ester, N-valine isopropyl ester and N-leucine isopropyl ester.

In some embodiments, R^{10A} can be

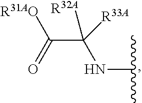

wherein R^{31A} can be selected from hydrogen, deuterium, an optionally substituted C₁₋₆-alkyl, an optionally substituted C₃₋₆ cycloalkyl, an optionally substituted aryl, an optionally substituted aryl (C₁₋₆ alkyl) and an optionally substituted haloalkyl; R^{32A} can be selected from hydrogen, deuterium, an optionally substituted C₁₋₆ alkyl, an optionally substituted C₁₋₆ haloalkyl, an optionally substituted C₃₋₆ cycloalkyl, an optionally substituted C₆ aryl, an optionally substituted C₁₀ aryl and an optionally substituted aryl (C₁₋₆ alkyl); and R^{33A} can be hydrogen, deuterium or an optionally substituted C₁₋₄-alkyl; or R^{32A} and R^{33A} can be taken together to form an optionally substituted C₃₋₆ cycloalkyl.

In some embodiments, R^{32A} can be substituted by a variety of substituents. Suitable examples of substituents include, but are not limited to, N-amido, mercapto, alkylthio, an optionally substituted aryl, hydroxyl, an optionally substituted heteroaryl, O-carboxy and amino. In some embodiments R^{32A} can be hydrogen or deuterium. In some embodiments, R^{32A} can be an optionally substituted C₁₋₆-alkyl. In some embodiments, R^{33A} can be hydrogen or deuterium. In some embodiments R^{33A} can be an optionally substituted C₁₋₄ alkyl, such as methyl, ethyl, n-propyl, isopropyl, n-butyl, isobutyl or tert-butyl. In some embodiments R^{33A} can be methyl. In some embodiments. R^{31A} can be an optionally substituted C₁₋₆ alkyl. Examples of optionally substituted C₁₋₆-alkyls include optionally substituted variants of the following: methyl, ethyl, n-propyl, isopropyl, n-butyl, isobutyl, tert-butyl, pentyl (branched and straight-chained) and hexyl (branched and straight-chained). In some embodiments, R^{31A} can be methyl or isopropyl. In some embodiments, R^{31A} can be ethyl or neopentyl. In some embodiments, R^{31A} can be an optionally substituted. C₃₋₆ cycloalkyl. Examples of optionally substituted C₃₋₆ cycloalkyls include optionally substituted variants of the following: cyclopropyl, cyclobutyl, cyclopentyl and cyclohexyl. Depending on the groups that are selected for R^{32A} and R^{33A}, the carbon to which R^{32A} and R^{33A} are attached may be a chiral center. In some embodiments, the carbon to which R^{32A} and R^{33A} are attached may be a (R)-chiral center. In other embodiments, the carbon to which R^{32A} and R^{33A} are attached may be a (S)-chiral center.

Examples of suitable

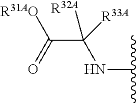

groups include the following:

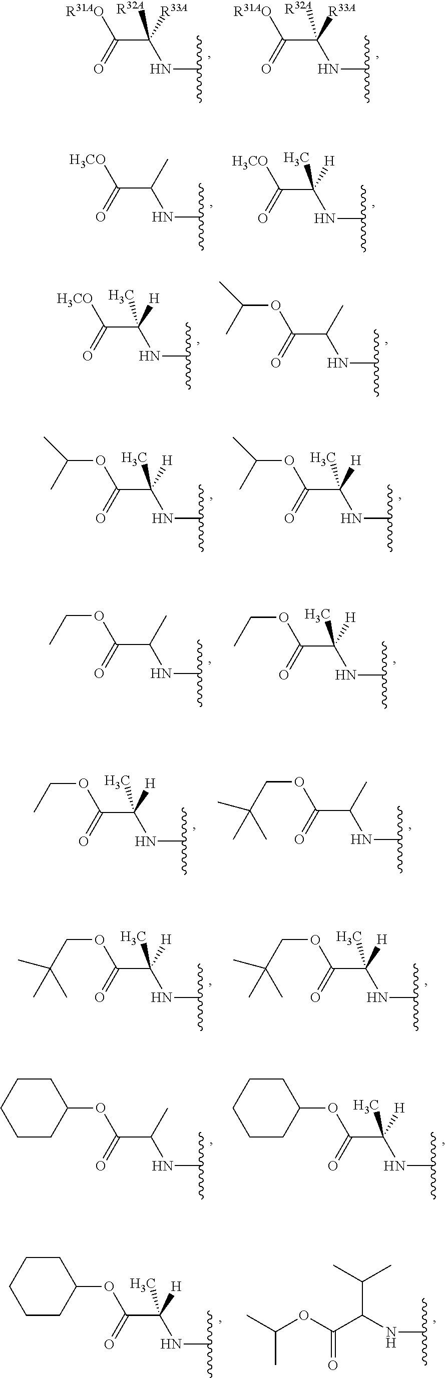

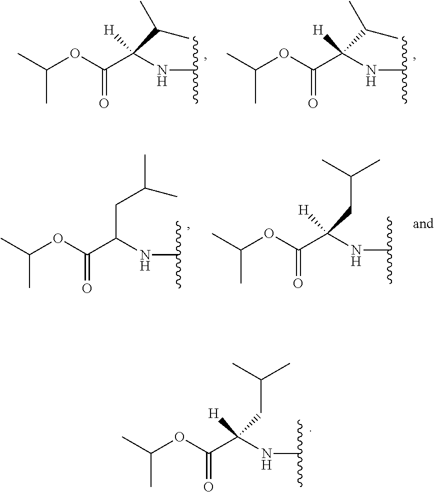

In some embodiments, R^{9A} and R^{10A} can form an optionally substituted phosphoramidate prodrug, such as an optionally substituted aryl phosphoramidate prodrug. For example, R^{9A} can be an —O-optionally substituted aryl and R^{10A} can be an optionally substituted N-linked amino acid or an optionally substituted N-linked amino acid ester derivative.

In some embodiments, both R^{9A} and R^{10A} can be independently an optionally substituted N-linked amino acid or an optionally substituted N-linked amino acid ester derivative for example, both R^{9A} and R^{10A} can be an optionally substituted N-linked amino acid or an optionally substituted N-linked α-amino acid ester derivative. Various amino acids are suitable, including those described herein. Examples of suitable amino acids include, but are not limited to, alanine, asparagine, aspartate, cysteine, glutamate, glutamine, glycine, proline, serine, tyrosine, arginine, histidine, isoleucine, leucine, lysine, methionine, phenylalanine, threonine, tryptophan and valine. In other embodiments, both R^{9A} and R^{10A} can be independently an optionally substituted N-linked amino acid ester derivative. Examples of suitable amino acid ester derivatives include, but are not limited to, an ester derivative of any of the following amino acids, alanine, asparagine, aspartate, cysteine, glutamate, glutamine, glycine, proline, serine, tyrosine, arginine, histidine, isoleucine, leucine, lysine, methionine, phenylalanine, threonine, tryptophan and valine. Additional examples of N-linked amino acid ester derivatives include, but are not limited to, an ester derivative of any of the following amino acids: alpha-ethyl-glycine, alpha-propyl-glycine and beta-alanine. In some embodiments, the N-linked amino acid ester derivative can be selected from N-alanine isopropyl ester, N-alanine cyclohexyl ester, N-alanine neopentyl ester, N-valine isopropyl ester and N-leucine isopropyl ester. In some embodiments, R^{9A} and R^{10A} can form an optionally substituted phosphonic diamide prodrug.

In some embodiments, both R^{9A} and R^{10A} can be independently

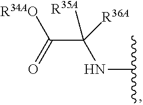

wherein R^{34A} can be selected from hydrogen, deuterium, an optionally substituted C₁₋₆-alkyl, an optionally substituted C₃₋₆ cycloalkyl, an optionally substituted aryl, an optionally substituted aryl (C₁₋₆ alkyl) and an optionally substituted haloalkyl; R^{35A} can be selected from hydrogen, deuterium, an optionally substituted C₁₋₆ alkyl, an optionally substituted C₁₋₆ haloalkyl, an optionally substituted C₃₋₆ cycloalkyl, an optionally substituted C₆ aryl, an optionally substituted C₁₀ aryl and an optionally substituted aryl (C₁₋₆ alkyl); and R^{36A} can be hydrogen, deuterium or an optionally substituted C₁₋₄-alkyl; or R^{35A} and R^{36A} can be taken together to form an optionally substituted C₃₋₆ cycloalkyl.

In some embodiments, R^{35A} can be substituted by a variety of substituents. Suitable examples of substituents include, but are not limited to, N-amido, mercapto, alkylthio, an optionally substituted aryl, hydroxyl, an optionally substituted heteroaryl, O-carboxy and amino. In some embodiments R^{35A} can be hydrogen or deuterium. In some embodiments, R^{35A} can be an optionally substituted C₁₋₆-alkyl. In some embodiments, R^{36A} can be hydrogen or deuterium. In some embodiments R^{36A} can be an optionally substituted C₁₋₄ alkyl, such as methyl, ethyl, n-propyl, isopropyl, n-butyl, isobutyl or tert-butyl. In some embodiments R_{36A} can be methyl. In some embodiments, R^{34A} can be an optionally substituted C₁₋₆ alkyl. Examples of optionally substituted C₁₋₆-alkyls include optionally substituted variants of the following: methyl, ethyl, n-propyl, isopropyl, n-butyl, isobutyl, tert-butyl, pentyl (branched and straight-chained) and hexyl (branched and straight-chained). In some embodiments, R^{34A} can be methyl or isopropyl. In some embodiments, R^{34A} can be ethyl or neopentyl. In some embodiments, R^{34A} can be an optionally substituted C₃₋₆ cycloalkyl. Examples of optionally substituted C₃₋₆ cycloalkyls include optionally substituted variants of the following: cyclopropyl, cyclobutyl, cyclopentyl and cyclohexyl. Depending on the groups that are selected for R^{35A} and R^{36A} the carbon to which R^{35A} and R^{36A} are attached may be a chiral center. In some embodiments, the carbon to which R^{35A} and R^{36A} are attached may be a (R)-chiral center. In other embodiments, the carbon to which R^{35A} and R^{36A} are attached may be a (S)-chiral center.

Examples of suitable

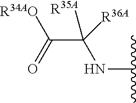

groups include the following

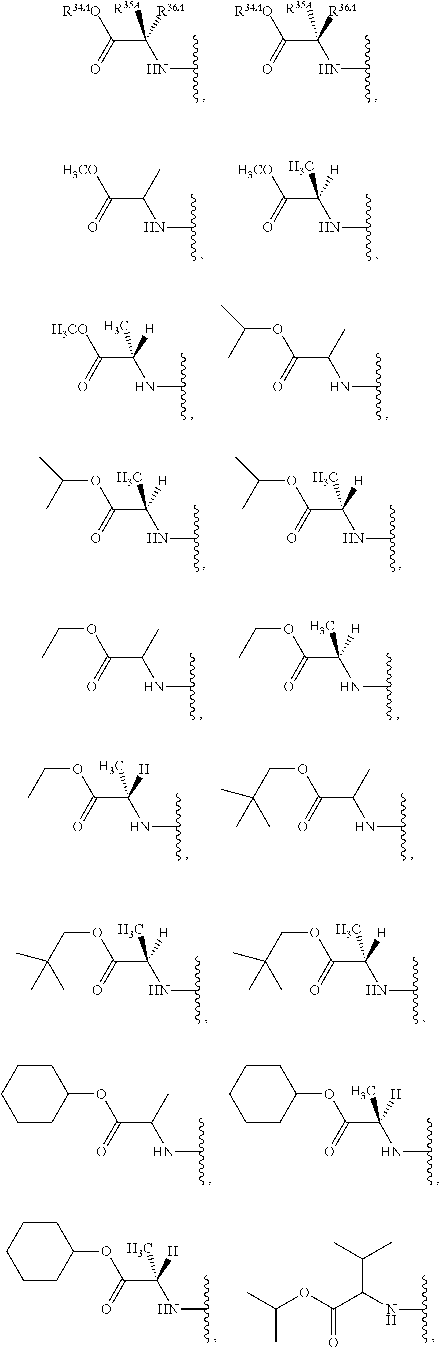

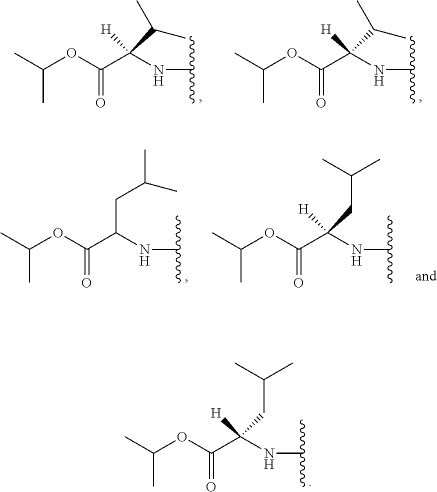

In some embodiments, R^{9A} and R^{10A} can be the same. In some embodiments, R^{9A} and R^{10A} can be different,

In some embodiments, R^{9A} and R^{10A} can be independently O⁻ or —OH. In other embodiments, R^{9A} can be

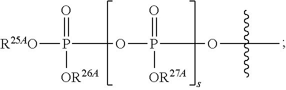

wherein s can be 0; R^{25A} and R^{26A} can be independently absent, hydrogen or deuterium; and R^{10A} can be O⁻ or —OH. Those skilled in the art understand that when R^{25A}, R^{26A} and R^{27A} are absent, the associated oxygen can have a negative charge. For example, when R^{26A} is absent, then the associated oxygen can have a negative charge, such that R^{9A} can be

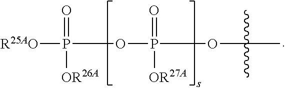

### When R^{9A} is

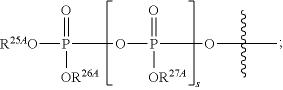

R^{25A} and R^{26A} are independently absent, hydrogen or deuterium, s is 0 and R^{10A} is O⁻ or —OH, a compound of Formula (I), or a pharmaceutically acceptable salt thereof, can be a diphosphate when Z^{1A} is O and an alpha-thiodiphosphate when Z^{1A} is S. In yet other embodiments R^{9A} can be

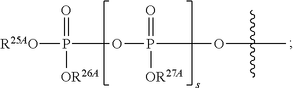

wherein s can be 1; R^{25A}, R^{26A} and R^{27A} can be independently absent, hydrogen or deuterium; and R^{10A} can be O⁻ or —OH. When R^{9A} is

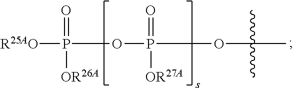

R^{25A}, R^{26A} and R^{27A} are independently absent, hydrogen or deuterium, s is 1 and R^{10A} is O⁻ or —OH, a compound of Formula (I), or a pharmaceutically acceptable salt thereof, can be a triphosphate when Z^{1A} is O and an alpha-thiotriphosphate when Z^{1A} is S.

In some embodiment, R^{6A} can be —OH. In other embodiment, R^{6A} can be OC(═O)R″^{A}, wherein R″^{A} can be an optionally substituted C₁₋₂₄ alkyl. In some embodiments, R″^{A} can be a substituted. C₁₋₁₂ alkyl. In other embodiments, R″^{A} can be an unsubstituted C₁₋₁₂ alkyl. In some embodiments, R″^{A} can be an unsubstituted C₁₋₈ alkyl.

In some embodiment, R^{6A} can be an optionally substituted O-linked amino acid, such as an optionally substituted O-linked α-amino acid. Examples of suitable O-linked amino acids are described herein and include alanine, asparagine, aspartate, cysteine, glutamate, glutamine, glycine, proline, serine, tyrosine, arginine, histidine, isoleucine, leucine, lysine, methionine, phenylalanine, threonine, tryptophan, valine, ornithine, hypusine, 2-aminoisobutyric acid, dehydroalanine, gamma-aminobutyric acid, citrulline, beta-alanine, alpha-ethyl-glycine, alpha-propyl-glycine and norleucine. In some embodiments, the O-linked amino acid can have the structure

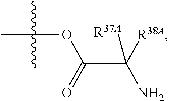

wherein R^{37A} can be selected from hydrogen, deuterium, an optionally substituted C₁₋₆ alkyl, an optionally substituted C₁₋₆ haloalkyl, an optionally substituted C₃₋₆ cycloalkyl, an optionally substituted C₆, aryl, an optionally substituted C₁₀ aryl and an optionally substituted aryl (C₁₋₆ alkyl); and R^{38A} can be hydrogen, deuterium or an optionally substituted C₁₋₄-alkyl; or R^{37A} and R^{38A} can be taken together to form an optionally substituted C₃₋₆ cycloalkyl.

When R^{37A} is substituted, R^{37A} can be substituted with one or more substituents selected from N-amino, mercapto, alkylthio, an optionally substituted aryl, hydroxy, an optionally substituted heteroaryl, O-carboxy and amino. In some embodiments, R^{37A} can be an unsubstituted C₁₋₆-alkyl, such as those described herein. In some embodiments, R^{37A} can be hydrogen or deuterium. In other embodiments, R^{37A} can be methyl. In some embodiments, R^{38A} can be hydrogen or deuterium. In other embodiments, R^{38A} can be an optionally substituted C₁₋₄-alkyl, such as methyl, ethyl, n-propyl, isopropyl, n-butyl, isobutyl and tert-butyl. In an embodiment, R^{38A} can be methyl. Depending on the groups that are selected for R^{37A} and R^{38A}, the carbon to which R^{37A} and R^{38A} are attached may be a chiral center. In some embodiment, the carbon to which R^{37A} and R^{38A} are attached may be a (R)-chiral center. In other embodiments, the carbon to which R^{37A} and R^{38A} are attached may be a (S)-chiral center.

Examples of suitable

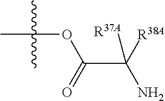

include the following

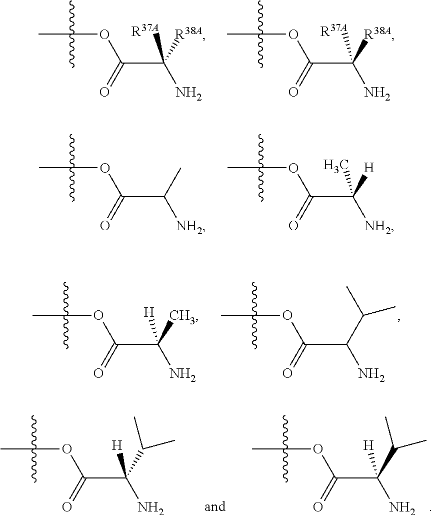

In some embodiments, R^{4A} can be hydrogen. In other embodiments, R^{4A} can be deuterium. In still other embodiments, R^{4A} can be fluoro.

At the 3′-position, in some embodiments, R^{5A} can be hydrogen. In other embodiments, R^{5A} can be deuterium. For the 1′-position, in some embodiments, R^{A} can be hydrogen. In other embodiments, R^{A} can be deuterium.

In some embodiments, R^{7A} can be —OH. In other embodiments, R^{7A} can be fluoro. In still other embodiments, R^{7A} can be chloro. In some embodiments, R^{7A} can be —OC(═O)R″^{B}. In some embodiments, R″^{B} can be a substituted C₁₋₁₂ alkyl. In other embodiments, R″^{B} can be an unsubstituted C₁₋₁₂ alkyl. In some embodiments, R″^{B} can be an unsubstituted C₁₋₈ alkyl.

In some embodiments, R^{8A} can be an optionally substituted C₂₋₆ allenyl or an unsubstituted C₂₋₆ allenyl. For example, R^{8A} can be —C═C═CH₂. In other embodiments, R^{8A} can be an optionally substituted C₂₋₆ alkynyl or an unsubstituted C₂₋₆ alkynyl. For example, R^{8A} can be ethynyl. In other embodiments, R^{8A} can be an optionally substituted C₁₋₃ alkyl. For example, R^{8A} can be methyl.

In some embodiments, R^{2A} can be hydrogen. In other embodiments, R^{2A} can be deuterium. In some embodiments, R^{3A} can be hydrogen. In other embodiments, R^{3A} can be deuterium. In some embodiments, R^{2A} and R^{3A} can each be hydrogen. In other embodiments, R^{2A} and R^{3A} can each be deuterium. In still other embodiments, one of R^{2A} and R^{3A} can be hydrogen and the other of R^{2A} and R^{3A} can be deuterium.

In some embodiments, B^{1A} can be adenine or an adenine derivative. As used herein, an adenine derivative refers to adenine that is substituted and/or in which one or more of the nitrogens in the bicyclic ring(s) is replaced with a CR^{C}, wherein R^{C} can be hydrogen, deuterium or any of the other substituents from the “optionally substituted” list. In some embodiments, B^{1A} can be guanine or an guanine derivative. As used herein, a guanine derivative refers to guanine that is substituted and/or in which one or more of the nitrogens in the bicyclic ring(s) is replaced with a CR^{C}, wherein R^{C} can be hydrogen, deuterium or any of the other substituents from the “optionally substituted” list. In some embodiments, B^{1A} is not an unsubstituted adenine or an unsubstituted guanine.

In some embodiments, B^{1A} can be

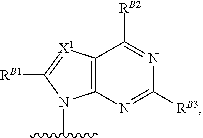

wherein X¹ can be N (nitrogen) or —CR^{B6}; R^{B1} can be hydrogen; R^{B2} can be NR^{B4a}R^{B4b}; R^{B3} can be hydrogen, halogen or NR^{B5a}R^{B5b}; R^{B4a}, R^{B4b}, R^{B5a} and R^{B5b} each be hydrogen; and R^{B6} can be hydrogen, halogen. —C≡N or —C(═O)NH₂.

In some embodiments, B^{1A} can be

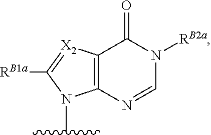

wherein X² can be N (nitrogen) or —CR^{B6a}; R^{B1a} can be hydrogen; R^{B2a} can be hydrogen or an optionally unsubstituted C₁₋₆ alkyl; and R^{B6a} can be hydrogen, halogen, —C≡N or —C(═O)NH₂.

In some embodiments, B^{1A} can be

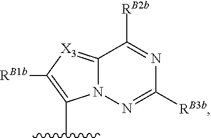

wherein X³ can be N (nitrogen) or —CR^{B6b}; R^{B1b} can be hydrogen; R^{B2b} can be NR^{B4a1}R^{B4b1}, R^{B3b} can be hydrogen, halogen or NR^{B5a1}R^{B5b1}; R^{B4a1}, R^{B4b1}, R^{B5a1} and R^{B5b1} can each be hydrogen; and R^{B6b} can be hydrogen, halogen, or —C≡N or —C(═O)NH₂.

In some embodiments, B^{1A} can be

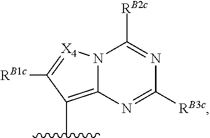

wherein

X⁴ can be N (nitrogen) or —CR^{B6c}; R^{B1c} can be hydrogen; R^{B2c} can be NR^{B4a2}R^{B4b2}; R^{B3c} can be hydrogen, halogen or NR^{B5a2}R^{B5b2}; R^{B4a2}, R^{B4b2}, R^{B5a2} and R^{B5b2} can each be hydrogen; and R^{B6c} can be hydrogen, halogen, —C≡N or —C(═O)NH₂.

In some embodiments, B^{1A} can be an optionally substituted

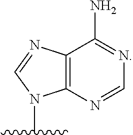 [SMILES: *n1cnc2c(N)ncnc21]

In some embodiments, B^{1A} can be an optionally substituted

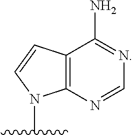 [SMILES: *n1ccc2c(N)ncnc21]

In some embodiments, B^{1A} can be an optionally substituted

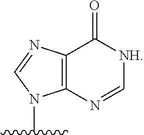 [SMILES: *n1cnc2c(=O)[nH]cnc21]

In some embodiments, B^{1A} can be an optionally substituted

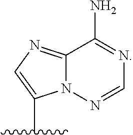 [SMILES: *c1cnc2c(N)ncnn12]

In some embodiments, B^{1A} can be an optionally substituted

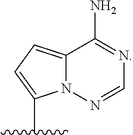 [SMILES: *c1ccc2c(N)ncnn12]

In some embodiments, B^{1A} can be an optionally substituted

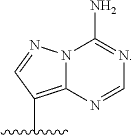 [SMILES: *c1cnn2c(N)ncnc12]

In some embodiments, B^{1A} can be an unsubstituted

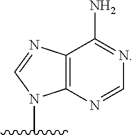 [SMILES: *n1cnc2c(N)ncnc21]

In some embodiments, B^{1A} can be a substituted

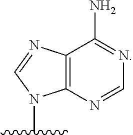 [SMILES: *n1cnc2c(N)ncnc21]

In some embodiments, B^{1A} can be an unsubstituted

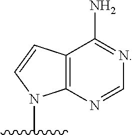 [SMILES: *n1ccc2c(N)ncnc21]

In some embodiments, B^{1A} can be a substituted

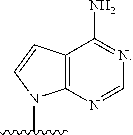 [SMILES: *n1ccc2c(N)ncnc21]

In some embodiments, B^{1A} can be a substituted

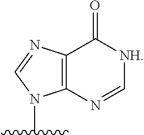 [SMILES: *n1cnc2c(=O)[nH]cnc21]

In some embodiments, B^{1A} can be an unsubstituted

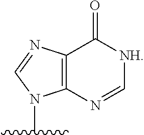 [SMILES: *n1cnc2c(=O)[nH]cnc21]

In some embodiments, B^{1A} can be a substituted

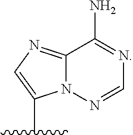 [SMILES: *c1cnc2c(N)ncnn12]

In some embodiments, B^{1A} can be an unsubstituted

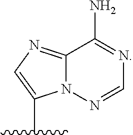 [SMILES: *c1cnc2c(N)ncnn12]

In some embodiments, B^{1A} can be a substituted

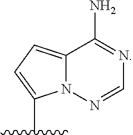 [SMILES: *c1ccc2c(N)ncnn12]

In some embodiments, B^{1A} can be an unsubstituted

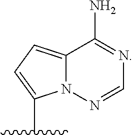 [SMILES: *c1ccc2c(N)ncnn12]

In some embodiments, B^{1A} can be a substituted

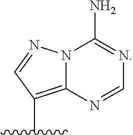 [SMILES: *c1cnn2c(N)ncnc12]

In some embodiments, B^{1A} can be an unsubstituted

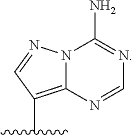 [SMILES: *c1cnn2c(N)ncnc12]

In some embodiments of this paragraph, the shown amino group (—NH₂) can replaced with a N-carbamyl group having the structure of —(NH)—(C═O)—OR″^{C}, wherein R″^{C} can be an optionally substituted C₁₋₆ alkyl. In some embodiments, R″^{C} can be an unsubstituted C₁₋₆ alkyl.

In some embodiments, B^{1A} can be selected from:

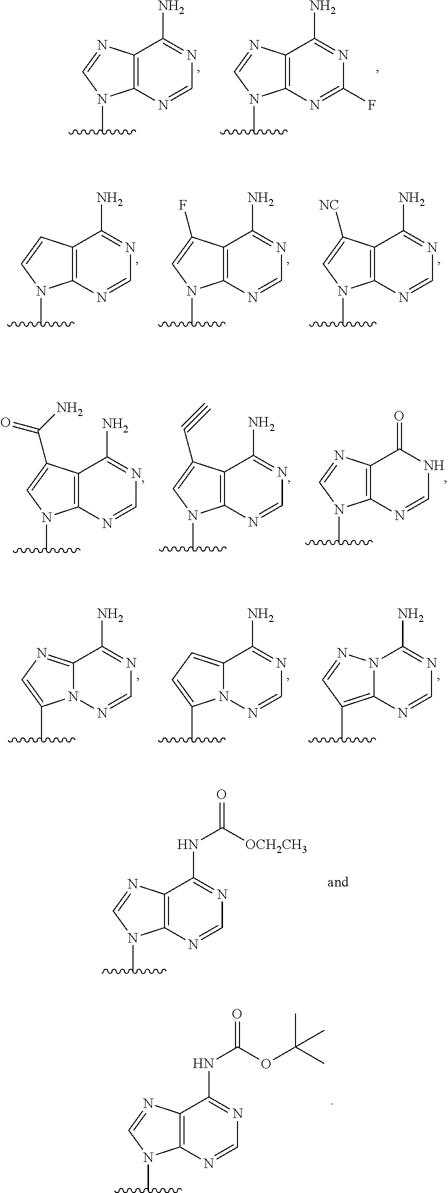

In some embodiments, R^{2A} can be hydrogen. In some embodiments, R^{2A} can be deuterium. In some embodiments, R^{3A} can be hydrogen. In some embodiments, R^{3A} can be deuterium. In some embodiments, R^{5A} can be hydrogen. In some embodiments, R^{5A} can be deuterium. In some embodiments, R^{2A} and R^{3A} can each be hydrogen. In some embodiments, R^{2A} and R^{3A} can each be deuterium

In some embodiments, R^{A} can be hydrogen. In some embodiments, R^{A} can be deuterium.

In some embodiments, when X¹ is N or CH, then (a) R^{4A} is fluoro, (b) R^{B3} is halogen or NR^{B5a}R^{B5b}, (c) R^{8A} is optionally substituted C₂₋₆ allenyl, or (d) any two or all three of said (a), (b) and (c) are present. In some embodiments when X¹ is N or CH, R^{4A} is fluoro and R^{1A} is hydrogen or triphosphate, then R^{8A} is not methyl. In some embodiments, the compound of Formula (I) is not selected from

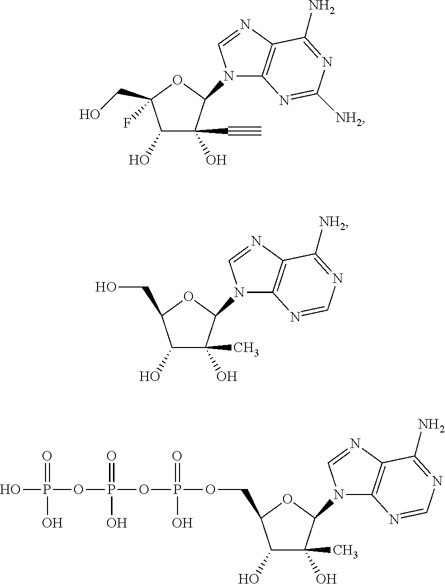 [SMILES: C#C[C@]1(O)[C@H](n2cnc3c(N)nc(N)nc32)O[C@](F)(CO)[C@H]1O ; C[C@@]1(O)[C@H](O)[C@@H](CO)O[C@H]1n1cnc2c(N)ncnc21 ; C[C@@]1(O)[C@H](O)[C@@H](COP(=O)(O)OP(=O)(O)OP(=O)(O)O)O[C@H]1n1cnc2c(N)ncnc21]

or a pharmaceutically acceptable salt of any of the foregoing.

In some embodiments, B^{1A} is not guanine or adenine. In some embodiments, when X¹ is N or CH, R^{4A} is fluoro and R^{1A} is hydrogen or triphosphate, then R^{8A} is not methyl. In some embodiments, when X¹ is N or CH, R^{4A} is fluoro and R^{8A} is methyl, then R^{B3} is halogen or NR^{B5a}R^{B5b}.

In some embodiments, X¹ can be N or —CR^{B6}, X² can be N (nitrogen) or —CR^{B6a}; X³ can be N (nitrogen) or —CR^{B6b}; X⁴ can be N (nitrogen) or —CR^{B6c}; R^{B1}, R^{B1a}, R^{B1b} and R^{B1c} can be hydrogen or deuterium; R^{B2} can be NR^{B4a}R^{B4b}; R^{B2b} can be NR^{B4a1}R^{B4b1}; R^{B2c} can be NR^{B4a2}R^{B4b2}; R^{B3} can be halogen or NR^{B5a}R^{B5b}; R^{B3b} can be halogen or NR^{B5a1}R^{B5b1}; R^{B3c} can be halogen or NR^{B5a2}R^{B5b2}; R^{B4a} and R^{B4b} can each be hydrogen; R^{B4a1} and R^{B4b1} can each be hydrogen; R^{B4a2} and R^{B4b2} can each be hydrogen; R^{B5a} and R^{B5b} can each be hydrogen; R^{B5a1} and R^{B5b1} can each be hydrogen; R^{B5a2} and R^{B5b2} can each be hydrogen; R^{B6}, R^{B6a}, R^{B6b} and R^{B6c} can be hydrogen or deuterium; R^{1A} can be hydrogen, an optionally substituted acyl, an optionally substituted O-linked amino acid or

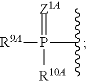

and R^{A} can be independently hydrogen or deuterium; R^{4A} can be fluoro; R^{6A} can be selected from —OH, —OC(═O)R″^{A} and an optionally substituted O-linked amino acid; R^{7A} can be —OH, fluoro or chloro; R^{8A} can be an optionally substituted C₁₋₃ alkyl, an optionally substituted C₂₋₆ allenyl or an optionally substituted C₂₋₆ alkynyl; R^{9A} and R^{10A} can be independently selected from O⁻, —OH, an optionally substituted —O—C₁₋₂₄ alkyl, an optionally substituted —O—C₂₋₂₄ alkenyl, an optionally substituted —O—C₂₋₂₄ alkynyl, an optionally substituted —O—C₃₋₆ cycloalkyl, an optionally substituted —O—C₅₋₁₀ cycloalkenyl, an optionally substituted —O-aryl, an optionally substituted —O-heteroaryl, an optionally substituted —O-aryl (C₁₋₆ alkyl), an optionally substituted *—O—(CR^{11A}R^{12A})ₚ—O—C₁₋₂₄ alkyl, an optionally substituted *—O—(CR^{13A}R^{14A})_{q}—O—C₁₋₂₄ alkenyl,

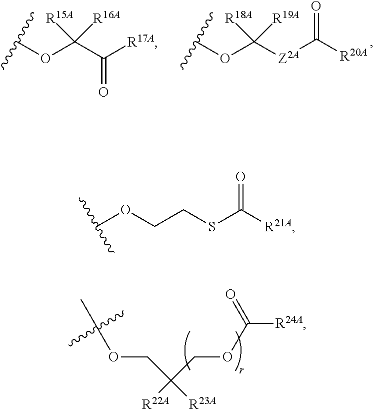

an optionally substituted N-linked amino acid or an optionally substituted N-linked amino acid ester derivative; or R^{9A} can be

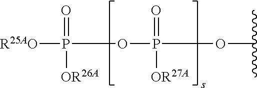

and R^{10A} can be O⁻ or OH; or R^{9A} and R^{10A} can be taken together to form a moiety selected from an optionally substituted

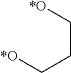

and an optionally substituted,

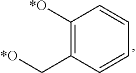

wherein the phosphorus and the moiety form a six-membered to ten-membered ring system; each R^{11A}, each R^{12A}, each R^{13A} and each R^{14A} are independently hydrogen, deuterium, an optionally substituted C₁₋₂₄ alkyl or alkoxy; R^{15A}, R^{16A}, R^{18A} and R^{19A} can be independently selected from hydrogen, deuterium, an optionally substituted C₁₋₂₄ alkyl and an optionally substituted aryl; R^{17A} and R^{20A} can be independently selected from hydrogen, deuterium, an optionally substituted C₁₋₂₄ alkyl, an optionally substituted aryl, an optionally substituted —O—C₁₋₂₄ alkyl, an optionally substituted —O-aryl, an optionally substituted —O-heteroaryl, an optionally substituted —O-monocyclic heterocyclyl; R^{21A} can be selected from hydrogen, deuterium, an optionally substituted C₁₋₂₄ alkyl and an optionally substituted aryl; R^{22A} and R^{23A} can be independently selected from —C≡N, an optionally substituted C₂₋₈ organylcarbonyl, an optionally substituted C₂₋₈ alkoxycarbonyl and an optionally substituted C₂₋₈ organylaminocarbonyl; R^{24A} can be selected from hydrogen, deuterium, an optionally substituted C₁₋₂₄-alkyl, an optionally substituted C₂₋₂₄ alkenyl, an optionally substituted C₂₋₂₄ alkynyl, an optionally substituted C₃₋₆ cycloalkyl and an optionally substituted C₅₋₁₀ cycloalkenyl; R^{25A}, R^{26A} and R^{27A} can be independently absent, hydrogen or deuterium; p and q can be independently selected from 1, 2 and 3; r can be 1 or 2; s can be 0 or 1; R″^{A} can be an optionally substituted C₁₋₂₄ alkyl; and Z^{1A} and Z^{2A} can be independently oxygen (O) or sulfur (S); and provided that the compound of Formula (I) is not selected from

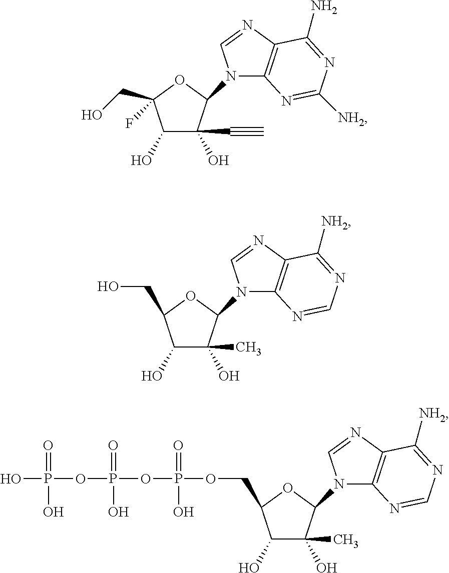 [SMILES: C#C[C@]1(O)[C@H](n2cnc3c(N)nc(N)nc32)O[C@](F)(CO)[C@H]1O ; C[C@@]1(O)[C@H](O)[C@@H](CO)O[C@H]1n1cnc2c(N)ncnc21 ; C[C@@]1(O)[C@H](O)[C@@H](COP(=O)(O)OP(=O)(O)OP(=O)(O)O)O[C@H]1n1cnc2c(N)ncnc21]

and a pharmaceutically acceptable salt thereof. In this paragraph, the asterisks indicate the points of attachment of the moieties.

In some embodiments, X¹ can be N or —CR^{B6}, R^{B1} can be hydrogen or deuterium; R^{B2} can be NR^{B4a}R^{B4b}; R^{B3} can be halogen or NR^{B5a}R^{B5b}; R^{B4a} and R^{B4b} can each be hydrogen; R^{B5a} and R^{B5b} can each be hydrogen; R^{B6} can be hydrogen or deuterium; R^{1A} can be hydrogen, an optionally substituted acyl, an optionally substituted O-linked amino acid or

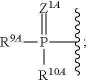

R^{2A}, R^{3A}, R^{5A} and R^{A} can be independently hydrogen or deuterium; R^{4A} can be fluoro; R^{6A} can be selected from —OH, —OC(═O)R″^{A} and an optionally substituted O-linked amino acid; R^{7A} can be —OH, fluoro or chloro; R^{8A} can be an optionally substituted C₁₋₃ alkyl, an optionally substituted C₂₋₆ alkenyl or an optionally substituted C₂₋₆ alkynyl; R^{9A} and R^{10A} can be independently selected from O⁻, —OH, an optionally substituted —O—C₁₋₂₄ alkyl, an optionally substituted —O—C₂₋₂₄ alkenyl, an optionally substituted —O—C₂₋₂₄ alkynyl, an optionally substituted —O—C₃₋₆ cycloalkyl, an optionally substituted —O—C₅₋₁₀ cycloalkenyl, an optionally substituted —O-aryl, an optionally substituted —O-heteroaryl, an optionally substituted —O-aryl (C₁₋₆ alkyl), an optionally substituted *—O—(CR^{11A}R^{12A})ₚ—O—C₁₋₂₄ alkyl, an optionally substituted *—O—(CR^{13A}R^{14A})_{q}—O—C₁₋₂₄ alkenyl,

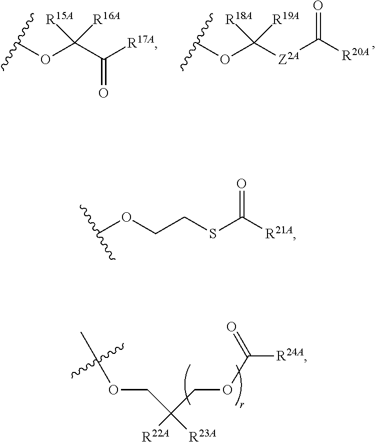

an optionally substituted N-linked amino acid or an optionally substituted N-linked amino acid ester derivative; or R^{9A} can be

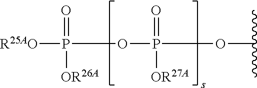

and R^{10A} can be O⁻ or OH; or R^{9A} and R^{10A} can be taken together to form a moiety selected from an optionally substituted

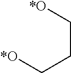

and an optionally substituted

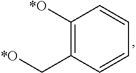

wherein the phosphorus and the moiety form a six-membered to ten-membered ring system; each R^{11A}, each R^{12A}, each R^{13A} and each R^{14A} can be independently hydrogen, deuterium, an optionally substituted C₁₋₂₄ alkyl or alkoxy; R^{15A}, R^{16A} and R^{19A} can be independently selected from hydrogen, deuterium, an optionally substituted C₁₋₂₄ alkyl a.nd an optionally substituted aryl; R^{17A} and R^{20A} can be independently selected from hydrogen, deuterium, an optionally substituted C₁₋₂₄ alkyl, an optionally substituted aryl, an optionally substituted —O—C₁₋₂₄ alkyl, an optionally substituted —O-aryl, an optionally substituted —O-heteroaryl, an optionally substituted —O-monocyclic heterocyclyl; R^{21A} can be selected from hydrogen, deuterium, an optionally substituted C₁₋₂₄ alkyl and an optionally substituted aryl; R^{27A} and R^{23A} can be independently selected from —C≡N, an optionally substituted C₂₋₈ organylcarbonyl, an optionally substituted C₂₋₈ alkoxycarbonyl and an optionally substituted C₂₋₈ organylaminocarbonyl; R^{24A} can be selected from hydrogen, deuterium, an optionally substituted C₁₋₂₄-alkyl, an optionally substituted C₂₋₂₄ alkenyl, an optionally substituted C₂₋₂₄ alkynyl, an optionally substituted C₃₋₆ cycloalkyl and an optionally substituted C₅₋₁₀ cycloalkenyl; R^{25A}, R^{26A} and R^{27A} can be independently absent, hydrogen or deuterium; p and q can be independently selected from 1, 2 and 3; r can be 1 or 2; s can be 0 or 1; R″^{A} can be an optionally substituted C₁₋₂₄ alkyl; and Z^{1A} and Z^{2A} can be independently oxygen (O) or sulfur (S); and provided that the compound of Formula (I) is not

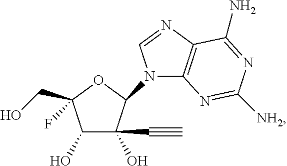 [SMILES: C#C[C@]1(O)[C@H](n2cnc3c(N)nc(N)nc32)O[C@](F)(CO)[C@H]1O]

or a pharmaceutically acceptable salt thereof. In this paragraph, the asterisks indicate the points of attachment of the moieties.

In some embodiments, X¹ can be N (nitrogen) or —CR^{B6}, X² can be N (nitrogen) or —CR^{B6a}; X³ can be N (nitrogen) or —CR^{B6b}; X⁴ can be N (nitrogen) or —CR^{B6c}; R^{B1}, R^{B1a}, R^{B1b} and R^{B1c} can be hydrogen or deuterium; R^{B2} can be NR^{B4a}R^{B4b}; R^{B2b} can be NR^{B4a1}R^{B4b1}; R^{B2c} can be NR^{B4a2}R^{B4b2}; R^{B3} can be hydrogen, deuterium, halogen or NR^{B5a}R^{B5b}, R^{B3b} can be hydrogen, deuterium, halogen or NR^{B5a1}R^{B5b1}; R^{B3c} can be hydrogen, deuterium, halogen or NR^{B5a2}R^{B5b2}; R^{B4a}, R^{B4a1} and R^{B4a2} can be independently hydrogen or deuterium; R^{B4b}, R^{B4b1} and R^{B4b2} can be independently selected from hydrogen, deuterium, an optionally substituted C₁₋₆ alkyl, an optionally substituted C₃₋₆ alkenyl, an optionally substituted C₃₋₆ cycloalkyl, —C(═O)R^{B7} and —C(═O)OR^{B8}; R^{B5a} can be hydrogen or deuterium; R^{B5a} can be selected from hydrogen, an optionally substituted C₁₋₆ alkyl, an optionally substituted C₃₋₆ alkenyl, an optionally substituted C₃₋₆ cycloalkyl, —C(═O)R^{B9} and —C(═O)OR^{B10}; R^{B6}, R^{B6a}, R^{B6b} and R^{B6c} can be selected from hydrogen, deuterium, halogen, —C≡N, —C(═O)NH₂, an optionally substituted C₁₋₆ alkyl, an optionally substituted C₂₋₆ alkenyl and an optionally substituted C₂₋₆ alkynyl; R^{B7}, R^{B8}, R^{B9} and R^{B10} can be independently selected from C₁₋₆ alkyl, C₂₋₆ alkenyl, C₂₋₆ alkynyl, C₃₋₆ cycloalkyl, C₅₋₁₀ cycloalkenyl, C₆₋₁₀ aryl, heteroaryl, heterocyclyl, aryl (C₁₋₆ alkyl), heteroaryl (C₁₋₆ alkyl) and heterocyclyl (C₁₋₆ alkyl); R^{1A} can be hydrogen, an optionally substituted acyl, an optionally substituted O-linked amino acid or

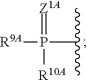

R^{2A}, R^{3A}, R^{5A} and R^{A} can be independently hydrogen or deuterium; R^{4A} can be hydrogen, deuterium or fluoro, R^{6A} can be selected from —OH, —OC(═O)R″^{A} and an optionally substituted O-linked amino acid; R^{7A} can be —OH, fluoro or chloro; R^{8A} can be an optionally substituted C₁₋₃ alkyl, an optionally substituted C₂₋₆ allenyl or an optionally substituted C₂₋₆ alkynyl; R^{9A} and R^{10A} can be independently selected from O⁻, —OH, an optionally substituted —O—C₁₋₂₄ alkyl, an optionally substituted —O—C₂₋₂₄ alkenyl, an optionally substituted —O—C₂₋₂₄ alkynyl, an optionally substituted —O—C₃₋₆ cycloalkyl, an optionally substituted —O—C₅₋₁₀ cycloalkenyl, an optionally substituted —O-aryl, an optionally substituted —O-heteroaryl, an optionally substituted —O-aryl (C₁₋₆ alkyl), an optionally substituted *—O—(CR^{11A}R^{12A})ₚ—O—C₁₋₂₄ alkyl, an optionally substituted *—O—(CR^{13A}R^{14A})_{q}—O—C₁₋₂₄ alkenyl,

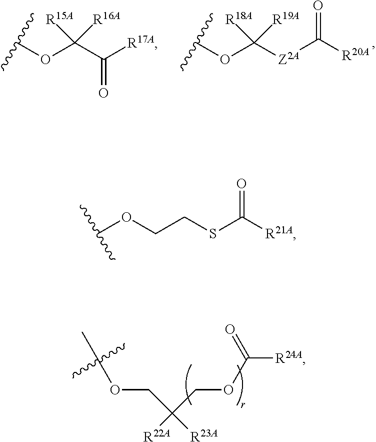

an optionally substituted N-linked amino acid or an optionally substituted N-linked amino acid ester derivative; or R^{9A} can be

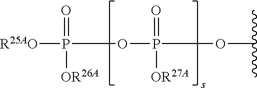

and R^{10A} can be O⁻ or OH; or R^{9A} and R^{10A} can be taken together to form a moiety selected from an optionally substituted

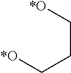

and an optionally substituted

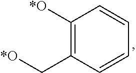

wherein the phosphorus and the moiety form a six-membered to ten-membered ring system; each R^{11A}, each R^{12A}, each R^{13A} and each R^{14A} can be independently hydrogen, deuterium, an optionally substituted C₁₋₂₄ alkyl or alkoxy; R^{16A}, R^{18A} and R^{19A} can be independently selected from hydrogen, deuterium, an optionally substituted C₁₋₂₄ alkyl and an optionally substituted aryl; R^{17A} and R^{20A} can be independently selected from hydrogen, deuterium, an optionally substituted C₁₋₂₄ alkyl, an optionally substituted aryl, an optionally substituted —O—C₁₋₂₄ alkyl, an optionally substituted —O-aryl, an optionally substituted —O-heteroaryl, an optionally substituted —O-monocyclic heterocyclyl; R^{21A} can be selected from hydrogen, deuterium, an optionally substituted C₁₋₂₄ alkyl and an optionally substituted aryl; R^{22A} and R^{23A} can be independently selected from C≡N, an optionally substituted C₂₋₈ organylcarbonyl, an optionally substituted C₂₋₈ alkoxycarbonyl and an optionally substituted C₂₋₈ organylaminocarbonyl; R^{24A} can be selected from hydrogen, deuterium, an optionally substituted C₁₋₂₄-alkyl, an optionally substituted C₂₋₂₄ alkenyl, an optionally substituted C₂₋₂₄ alkynyl, an optionally substituted C₃₋₆ cycloalkyl and an optionally substituted C₅₋₁₀ cycloalkenyl; R^{25A}, R^{26A} and R^{27A} can be independently absent, hydrogen or deuterium; p and q can be independently selected from 1, 2 and 3; r can be 1 or 2; s can be 0 or 1; R″^{A} can be an optionally substituted C₁₋₂₄ alkyl; and Z^{1A} and Z^{2A} can be independently oxygen (O) or sulfur (S); and provided that when X¹ is N or CH, then (a) R^{4A} is fluoro, (b) R^{B3} is halogen or NR^{B5a}R^{B5b}, (c) R^{8A} is optionally substituted C₂₋₆ allenyl, or (d) any two or all three of said (a), (b) and (c) are present; and provided that when X¹ is N or CH, R^{4A} is fluoro and R^{1A} is hydrogen or triphosphate, then R^{8A} is not methyl; and provided that the compound of Formula (I) is not selected from

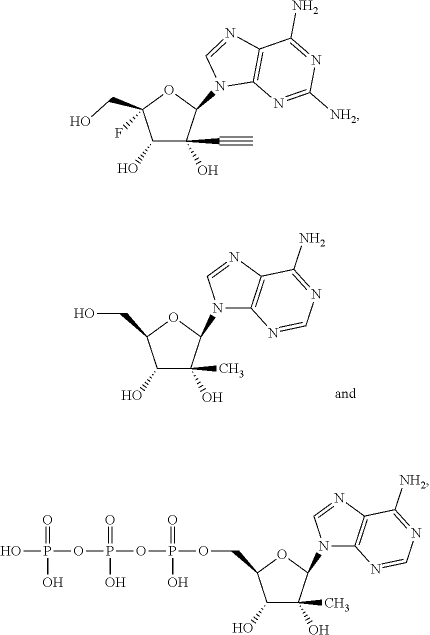 [SMILES: C#C[C@]1(O)[C@H](n2cnc3c(N)nc(N)nc32)O[C@](F)(CO)[C@H]1O ; C[C@@]1(O)[C@H](O)[C@@H](CO)O[C@H]1n1cnc2c(N)ncnc21 ; C[C@@]1(O)[C@H](O)[C@@H](COP(=O)(O)OP(=O)(O)OP(=O)(O)O)O[C@H]1n1cnc2c(N)ncnc21]

and a pharmaceutically acceptable salt thereof. In this paragraph, the asterisks indicate the points of attachment of the moieties.

In some embodiments, X¹ can be N (nitrogen) or —CR^{B6}, X² can be N (nitrogen) or —CR^{B6a}; X³ can be N (nitrogen) or —CR^{B6b}; X⁴ can be N (nitrogen) or —CR^{B6c}; R^{B1}, R^{B1a}, R^{B1b} and R^{B1c} can be hydrogen or deuterium; R^{B2} can be NR^{B4a}R^{B4b}; R^{B2b} can be NR^{B4a1}R^{B4b1}; R^{B2c} can be NR^{B4a2}R^{B4b2}; R^{B3} can be hydrogen, deuterium, halogen or NR^{B5a}R^{B3b}; R^{B3b} can be hydrogen, deuterium, halogen or NR^{B5a1}R^{B5b1}; R^{B3c} can be hydrogen, deuterium, halogen or NR^{B5a2}R^{B5b2}; R^{B4a}, R^{B4a1} and R^{B4a2} can be independently hydrogen or deuterium; R^{B4b}, R^{B4b1} and R^{B4b2} can be independently selected from hydrogen, deuterium an optionally substituted C₁₋₆ alkyl, an optionally substituted C₃₋₆ alkenyl, an optionally substituted C₃₋₆ cycloalkyl, —C(═O)R^{B7} and —C(═O)OR^{B8}; R^{B5a} can be hydrogen or deuterium; R^{B5a} can be selected from hydrogen, an optionally substituted C₁₋₆ alkyl, an optionally substituted C₃₋₆ alkenyl, an optionally substituted C₃₋₆ cycloalkyl, —C(═O)R^{B7} and —C(═O)OR^{B10}; R^{B6}, R^{B6a}, R^{B6b} and R^{B6c} can be selected from hydrogen, deuterium, halogen, —C≡N, —C(═O)NH₂, an optionally substituted C₁₋₆ alkyl, an optionally substituted C₂₋₆ alkenyl and an optionally substituted C₂₋₆ alkynyl; R^{B7}, R^{B8}, R^{B9} and R^{B10} can be independently selected from C₁₋₆ alkyl, C₂₋₆ alkenyl, C₂₋₆ alkynyl, C₃₋₆ cycloalkyl, C₅₋₁₀ cycloalkenyl, C₆₋₁₀ aryl, heteroaryl, heterocyclyl, aryl (C₁₋₆ alkyl), heteroaryl (C₁₋₆ alkyl) and heterocyclyl (C₁₋₆ alkyl); R^{1A} can be hydrogen, an optionally substituted acyl, an optionally substituted O-linked amino acid or

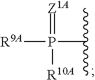

R^{2A}, R^{3A}, R^{5A} and R^{A} can be independently hydrogen or deuterium; R^{4A} can be hydrogen, deuterium or fluoro; R^{6A} can be selected from —OH, —OC(═O)R″^{A} and an optionally substituted O-linked amino acid; R^{7A} can be fluoro or chloro; R^{8A} can be an optionally substituted C₂₋₆ allenyl of an optionally substituted C₂₋₆ alkynyl; R^{9A} and R^{10A} can be independently selected from O⁻, —OH, an optionally substituted —O—C₁₋₂₄ alkyl, an optionally substituted —O—C₂₋₂₄ alkenyl, an optionally substituted —O—C₂₋₂₄ alkynyl, an optionally substituted —O—C₃₋₆ cycloalkyl, an optionally substituted —O—C₅₋₁₀ cycloalkenyl, an optionally substituted —O-aryl, an optionally substituted —O-heteroaryl, an optionally substituted —O-aryl (C₁₋₆ alkyl), an optionally substituted *—O—(CR^{11A}R^{12A})ₚ—O—C₁₋₂₄ alkyl, an optionally substituted *—O—(CR^{13A}R^{14A})_{q}—O—C₁₋₂₄ alkenyl,

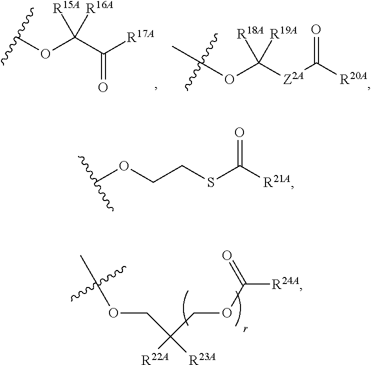

an optionally substituted N-linked amino acid or an optionally substituted N-linked amino acid ester derivative; or R^{9A} can be

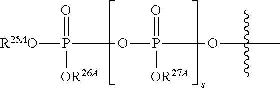

and R^{10A} can be O⁻ or OH; or R^{9A} and R^{10A} can be taken together to form a moiety selected from an optionally substituted

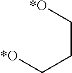

and an optionally substituted

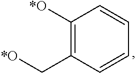

wherein the phosphorus and the moiety form a six-membered to ten-membered ring system; each R^{11A}, each R^{12A}, each R^{13A} and each R^{14A} can be independently hydrogen, deuterium, an optionally substituted C₁₋₂₄ alkyl or alkoxy; R^{15A}, R^{16A}, R^{18A} and R^{19A} can be independently selected from hydrogen, deuterium, an optionally substituted C₁₋₂₄ alkyl and an optionally substituted aryl; R^{17A} and R^{20A} can be independently selected from hydrogen, deuterium, an optionally substituted C₁₋₂₄ alkyl, an optionally substituted aryl, an optionally substituted —O—C₁₋₂₄ alkyl, an optionally substituted —O-aryl, an optionally substituted —O-heteroaryl, an optionally substituted —O-monocyclic heterocyclyl; R^{21A} can be selected from hydrogen, deuterium, an optionally substituted C₁₋₂₄ alkyl and an optionally substituted aryl; R^{22A} and R^{23A} can be independently selected from —C≡N, an optionally substituted C₂₋₈ organylcarbonyl, an optionally substituted C₂₋₈ alkoxycarbonyl and an optionally substituted C₂₋₈ organylaminocarbonyl; R^{24A} can be selected from hydrogen, deuterium, an optionally substituted C₁₋₂₄-alkyl, an optionally substituted C₂₋₂₄ alkenyl, an optionally substituted C₂₋₂₄ alkynyl, an optionally substituted C₃₋₆ cycloalkyl and an optionally substituted C₅₋₁₀ cycloalkenyl; R^{25A}, R^{26A} and R^{27A} can be independently absent, hydrogen or deuterium; p and q can be independently selected from 1, 2 and 3; r can be 1 or 2; s can be 0 or 1; R″^{A} can be an optionally substituted C₁₋₂₄ alkyl; and Z^{1A} and Z^{2A} can be independently oxygen (O) or sulfur (S). In some embodiments of this paragraph, when X¹ is N or CH, then (a) R^{4A} is fluoro, (b) R^{B3} is halogen or NR^{B5a}R^{B5b}, (c) R^{8A} is optionally substituted C₂₋₆ alkenyl, or (d) any two or all three of said (a), (b) and (c) are present. In some embodiments of this paragraph, the compound of Formula (I) is not

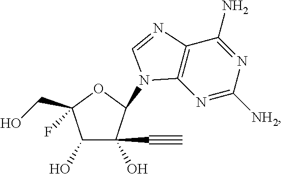 [SMILES: C#C[C@]1(O)[C@H](n2cnc3c(N)nc(N)nc32)O[C@](F)(CO)[C@H]1O]

and/or a pharmaceutically acceptable salt thereof. In this paragraph, the asterisks indicate the points of attachment of the moieties.

In some embodiments, X¹ can be N (nitrogen) or —CR^{B6}, X² can be N (nitrogen) or —CR^{B6a}; X³ can be N (nitrogen) or —CR^{B6b}; X⁴ can be N (nitrogen) or —CR^{B6c}; R^{B1}, R^{B1a}, R^{B1b} and R^{B1c} can be hydrogen or deuterium, R^{B2} can be NR^{B4a}R^{B4b}; R^{B2b} can be NR^{B4a1}R^{B4b1}; R^{B2c} can be NR^{B4a2}R^{B4b2}; R^{B3} can be hydrogen, deuterium, halogen or NR^{B5a}R^{B5b}; R^{B3b} can be hydrogen, deuterium, halogen or NR^{B5a1}R^{B5b1}; R^{B3c} can be hydrogen, deuterium, halogen or NR^{B5a2}R^{B5b2}; R^{B4a}, R^{B4a1} and R^{B4a2} can be indepdently hydrogen or deuterium; R^{B4b}, R^{B4b1} and R^{B4b2} can be independently selected from hydrogen, deuterium an optionally substituted C₁₋₆ alkyl, an optionally substituted C₃₋₆ alkenyl, an optionally substituted C₃₋₆ cycloalkyl, —C(═O)R^{B7} and —C(═O)OR^{B8}; R^{B6}, R^{B6b} and R^{B6c} can be selected from hydrogen, deuterium, halogen, —C≡N, —C(═O)NH₂, an optionally substituted C₁₋₆ alkyl, an optionally substituted C₂₋₆ alkenyl and an optionally substituted C₂₋₆ alkynyl; R^{B7}, R^{8B}, R^{B9} and R^{B10} can be independently selected from C₁₋₆ alkyl, C₂₋₆ alkenyl, C₂₋₆ alkynyl, C₃₋₆ cycloalkyl, C₅₋₁₀ cycloalkenyl, C₆₋₁₀ aryl, heteroaryl, heterocyclyl, aryl (C₁₋₆ alkyl), heteroaryl (C₁₋₆ alkyl) and heterocyclyl (C₁₋₆ alkyl); R^{1A} can be

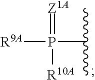

R^{2A}, R^{3A}, R^{5A} and R^{A} can be independently hydrogen or deuterium; R^{4A} can be hydrogen, deuterium or fluoro; R^{6A} can be selected from —OH, —OC(═O)R″^{A} and an optionally substituted O-linked amino acid; R^{7A} can be —OH, fluoro or chloro; R^{8A} can be an optionally substituted C₁₋₃ alkyl, an optionally substituted C₂₋₆ allenyl or an optionally substituted C₂₋₆ alkynyl; R^{9A} can be

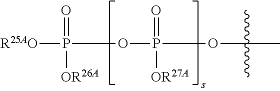

and R^{10A} can be O⁻ or OH; R^{25A}, R^{26A} and R^{27A} can be independently absent, hydrogen or deuterium; s can be 0 or 1; and Z^{1A} and Z^{2A} can be independently oxygen (O) or sulfur (S). In some embodiments of this paragraph, when X¹ is N or CH, then (a) R^{4A} is fluoro, (b) R^{B3} is halogen or NR^{B5a}R^{B5b}, (c) R^{8A} is optionally substituted C₂₋₆ allenyl, or (d) any two or all three of said (a). (b) and (c) are present. In some embodiments of this paragraph, when X¹ is N or CH, R^{4A} is fluoro and R^{1A} is triphosphate, then R^{8A} is not methyl. In some embodiments of this paragraph, the compound of Formula (I) is not

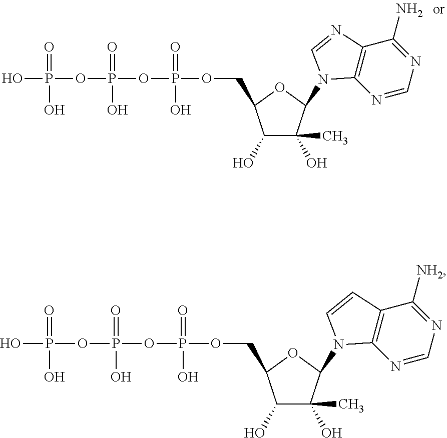 [SMILES: C[C@@]1(O)[C@H](O)[C@@H](COP(=O)(O)OP(=O)(O)OP(=O)(O)O)O[C@H]1n1cnc2c(N)ncnc21 ; C[C@@]1(O)[C@H](O)[C@@H](COP(=O)(O)OP(=O)(O)OP(=O)(O)O)O[C@H]1n1ccc2c(N)ncnc21]

and a pharmaceutically acceptable salt thereof. In some embodiments of this paragraph, R^{4A} can be hydrogen. In some embodiments of this paragraph, R^{4A} can be deuterium. In some embodiments of this paragraph, R^{4A} can be fluoro. In some embodiments of this paragraph, Z^{1A} can be O.

In some embodiments, a compound of Formula (I), or a pharmaceutically acceptable salt thereof can be wherein: B^{1A} can be

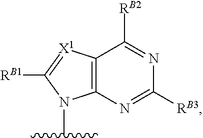

wherein: X¹ can be N (nitrogen) or —CR^{B6}; R^{B1} can be hydrogen or deuterium; R^{B2} can be NR^{B4a}R^{B4b}; R^{B3} can be hydrogen, deuterium, halogen or NR^{B5a}R^{B5b}; R^{B4a} can be hydrogen or deuterium; R^{B4b} can be selected from hydrogen, deuterium, an optionally substituted C₁₋₆ alkyl, an optionally substituted C₃₋₆ alkenyl, an optionally substituted C₃₋₆ cycloalkyl, —C(═O)R^{B7} and —C(═O)OR^{B8}; R^{B5a} can be hydrogen or deuterium; R^{B5a} can be selected from hydrogen, deuterium, an optionally substituted C₁₋₆ alkyl, an optionally substituted C₃₋₆ alkenyl, an optionally substituted C₃₋₆ cycloalkyl, —C(═O)R^{B9} and —C(═O)OR^{B10}; R^{B6} can be selected from hydrogen, deuterium, halogen, —C≡N, —C(═O)NH₂, an optionally substituted C₁₋₆ alkyl, an optionally substituted C₂₋₆ alkenyl and an optionally substituted C₂₋₆ alkynyl; R^{B7}, R^{B8}, R^{B9} and R^{B10} can be independently selected from C₁₋₆ alkyl, C₂₋₆ alkenyl, C₂₋₆ alkynyl, C₃₋₆ cycloalkyl, C₃₋₆ cycloalkenyl, C₆₋₁₀ aryl, heteroaryl, heterocyclyl, aryl (C₁₋₆ alkyl), heteroaryl (C₁₋₆ alkyl) and heterocyclyl (C₁₋₆ alkyl); R^{1A} can be hydrogen, deuterium, an optionally substituted acyl, an optionally substituted O-linked amino acid or

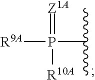

R^{2A}, R^{3A}, R^{5A} and R^{A} can be independently hydrogen or deuterium; R^{4A} can be hydrogen, deuterium or fluoro; R^{6A} can be selected from —OH, —OC(═O)R″^{A} and an optionally substituted O-linked amino acid; R^{7A} can be —OH, fluoro or chloro; R^{8A} can be an optionally substituted C₁₋₃ alkyl, an optionally substituted. C₂₋₆ allenyl or an optionally substituted C₂₋₆ alkynyl; R^{9A} and R^{10A} can be independently selected from O⁻, —OH, an optionally substituted —O—C₁₋₂₄ alkyl, an optionally substituted —O—C₂₋₂₄ alkenyl, an optionally substituted —O—₂₋₂₄ alkynyl, an optionally substituted —O—C₃₋₆ cycloalkyl, an optionally substituted —O—C₃₋₆ cycloalkenyl, an optionally substituted —O-aryl, an optionally substituted —O-heteroaryl, an optionally substituted —O-aryl (C₁₋₆ alkyl), an optionally substituted *—O—(CR^{11A}R^{12A})ₚ—O—C₁₋₂₄ alkyl, an optionally substituted *—O—(CR^{13A}R^{14A})_{q}—O—C₁₋₂₄ alkenyl,

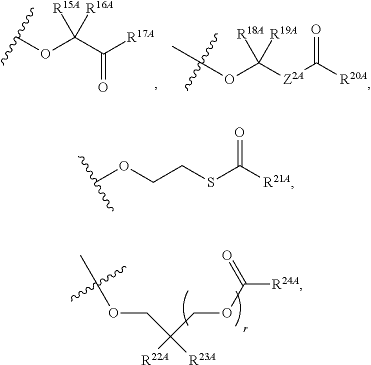

an optionally substituted N-linked amino acid or an optionally substituted N-linked amino acid ester derivative; or R^{9A} can be

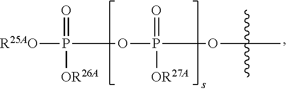

and R^{10A} can be O⁻ or OH; or R^{9A} and R^{10A} can be taken together to form a moiety selected from an optionally substituted

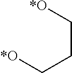

and an optionally substituted

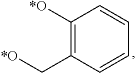

wherein the phosphorus and the moiety form a six-membered to ten-membered ring system; each R^{11A}, each R^{12A}, each R^{13A} and each R^{14A} can be independently hydrogen, deuterium, an optionally substituted C₁₋₂₄ alkyl or alkoxy; R^{15A}, R^{16A}, R^{18A} and R^{19A} can be independently selected from hydrogen, deuterium, an optionally substituted C₁₋₂₄ alkyl and an optionally substituted aryl; R^{17A} and R^{20A} can be independently selected from hydrogen, deuterium, an optionally substituted C₁₋₂₄ alkyl, an optionally substituted aryl, an optionally substituted —O—C₁₋₂₄ alkyl, an optionally substituted —O-aryl, an optionally substituted —O-heteroaryl, an optionally substituted —O-monocyclic heterocyclyl; R^{21A} can be selected from hydrogen, deuterium, an optionally substituted C₁₋₂₄ alkyl and an optionally substituted aryl; R^{22A} and R^{23A} can be independently selected from —C≡N, an optionally substituted C₂₋₈ organylcarbonyl, an optionally substituted C₂₋₈ alkoxycarbonyl and an optionally substituted C₂₋₈ organylaminocarbonyl; R^{24A} can be selected from hydrogen, deuterium, an optionally substituted C₁₋₂₄-alkyl, an optionally substituted C₂₋₂₄ alkenyl, an optionally substituted C₂₋₂₄ alkynyl, an optionally substituted C₃₋₆ cycloalkyl and an optionally substituted C₃₋₆ cycloalkenyl; R^{25A}, R^{26A} and R_{27A} can be independently absent, hydrogen or deuterium; p and q can be independently selected from 1, 2 and 3; r can be 1 or 2; s can be 0 or 1; R″^{A} can be an optionally substituted C₁₋₂₄ alkyl; and Z^{1A} and Z^{2A} can be independently oxygen (O) or sulfur (S). In this paragraph, the asterisks indicate the points of attachment of the moieties.

In some embodiments, a compound of Formula (I), or a pharmaceutically acceptable salt thereof can be wherein: B^{1A} can be

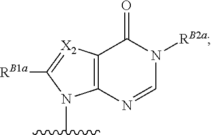

wherein: X² can be N (nitrogen) or —CR^{B6a}; R^{B1a} can be selected from hydrogen or deuterium; R^{B2a} can be NR^{B4a}R^{B4b}; R^{B3a} can be selected from hydrogen, deuterium, halogen or NR^{B5a}R^{B5b}; R^{B4a} can be hydrogen or deuterium; R^{B4b} can be selected from hydrogen, an optionally substituted C₁₋₆ alkyl, an optionally substituted C₂₋₆ alkenyl, an optionally substituted C₃₋₆ cycloalkyl, —C(═O)R^{B7} and —C(═O)OR^{B8}; R^{B5a} can be selected from hydrogen or deuterium; R^{B5b} can be selected from hydrogen, an optionally substituted C₁₋₆ alkyl, an optionally substituted C₂₋₆ alkenyl, an optionally substituted C₃₋₆ cycloalkyl, —C(═O)^{B9} and —C(═O)OR^{B10}; R^{B6a} can be selected from hydrogen, deuterium, halogen, —C≡N, —C(═O)NH₂, an optionally substituted C₁₋₆ alkyl, an optionally substituted C₂₋₆ alkenyl and an optionally substituted C₂₋₆ alkynyl; R^{B7}, R^{B8}, R^{B9} and R^{B10} can independently be selected from an optionally substituted C₁₋₆ alkyl, an optionally substituted C₂₋₆ alkenyl, an optionally substituted C₂₋₆ alkynyl, an optionally substituted C₃₋₆ cycloalkyl, an optionally substituted C₅₋₁₀ cycloalkenyl, an optionally substituted C₆₋₁₀ aryl, an optionally substituted heteroaryl, an optionally substituted heterocyclyl, an optionally substituted aryl (C₁₋₆ alkyl), an optionally substituted heteroaryl (C₁₋₆ alkyl) and an optionally substituted heterocyclyl (C₁₋₆ alkyl); R^{1A} can be hydrogen, an optionally substituted acyl, an optionally substituted O-linked amino acid or

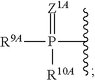

R^{2A}, R^{3A}, R^{5A} and R^{A} can independently be hydrogen or deuterium; R^{4A} can be hydrogen, deuterium or fluoro; R^{6A} can be selected from —OH, —OC(═O)R″^{A} and an optionally substituted O-linked amino acid; R^{7A} can be —OH, —OC(═O)R″^{B}, fluoro or chloro; R^{8A} can be an optionally substituted C₁₋₃ alkyl, an optionally substituted C₂₋₆ alkenyl or an optionally substituted C₂₋₆ alkynyl; R^{9A} and R^{10A} can independently be selected from O⁻, —OH, an optionally substituted —O—C₁₋₂₄ alkyl, an optionally substituted —O—C₂₋₂₄ alkenyl, an optionally substituted —O—C₂₋₂₄ alkynyl, an optionally substituted —O—C₃₋₆ cycloalkyl, an optionally substituted —O—C₅₋₁₀ cycloalkenyl, an optionally substituted —O-aryl, an optionally substituted —O-heteroaryl, an optionally substituted —O-aryl (C₁₋₆ alkyl), an optionally substituted *—O—(CR^{11A}R^{12A})ₚ—O—C₁₋₂₄ alkyl, an optionally substituted *—O—(CR^{13A}R^{14A})_{q}—O—C₂₋₂₄ alkenyl,

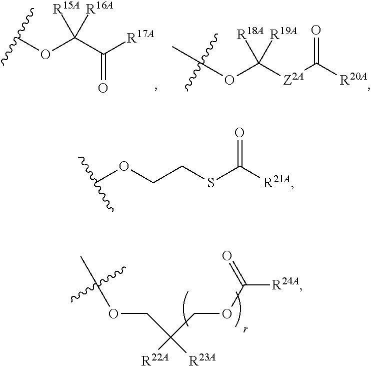

an optionally substituted N-linked amino acid or an optionally substituted N-linked amino acid ester derivative; or R^{9A} can be

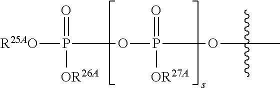

and R^{10A} can be O⁻ or OH; or R^{9A} and R^{10A} can be taken together to form a moiety selected from an optionally substituted

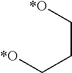

and an optionally substituted

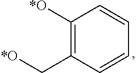

wherein the phosphorus and the moiety form a six-membered to ten-membered ring system; each R^{11A}, each R^{12A}, each R^{13A} and each R^{14A} can be independently hydrogen, deuterium, an optionally substituted C₁₋₂₄ alkyl or alkoxy; R^{15A}, R^{16A}, R^{18A} and R^{19A} can be independently selected from hydrogen, deuterium, an optionally substituted C₁₋₂₄ alkyl and an optionally substituted aryl; R^{17A} and R^{20A} can be independently selected from hydrogen, deuterium, an optionally substituted C₁₋₂₄ alkyl, an optionally substituted aryl, an optionally substituted —O—C₁₋₂₄ alkyl, an optionally substituted —O-aryl, an optionally substituted —O-heteroaryl and an optionally substituted —O-monocyclic heterocyclyl; R^{21A} can be selected from hydrogen, deuterium, an optionally substituted C₁₋₂₄ alkyl and an optionally substituted aryl; R^{22A} and R^{23A} can be independently selected from —C≡N, an optionally substituted C₂₋₈ organylcarbonyl, an optionally substituted C₂₋₈ alkoxycarbonyl and an optionally substituted C₂₋₈ organylaminocarbonyl; R^{24A} can be selected from hydrogen, deuterium, an optionally substituted C₁₋₂₄-alkyl, an optionally substituted C₂₋₂₄ alkenyl, an optionally substituted C₂₋₂₄ alkynyl, an optionally substituted C₂₋₆ cycloalkyl and an optionally substituted C₅₋₁₀ cycloalkenyl; R^{25A}, R^{26A} and R^{27A} can be independently absent, hydrogen or deuterium; p and q can be independently selected from 1, 2 and 3; r can be 1 or 2; s can be 0 or 1; R″^{A} and R″^{B} can be independently an optionally substituted C₁₋₂₄ alkyl; and Z^{1A} and Z^{2A} can be independently oxygen (O) or sulfur (S). In this paragraph, the asterisks indicate the points of attachment of the moieties.

In some embodiments, a compound of Formula (I), or a pharmaceutically acceptable salt thereof can be wherein: B^{1A} can be,

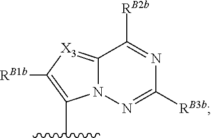

wherein, X³ can be N (nitrogen) or —CR^{B6b}; R^{B1b} can be selected from hydrogen or deuterium; R^{B2b} can be NR^{B4a1}R^{B4b1}; R^{B3b} can be selected from hydrogen, deuterium, halogen or NR^{B5a1}R^{B5b1}; R^{B4a1} can be hydrogen or deuterium; R^{B4b1} can be selected from hydrogen, deuterium, an optionally substituted C₁₋₆ alkyl, an optionally substituted C₂₋₆ alkenyl, an optionally substituted C₃₋₆ cycloalkyl, —C(═O)R^{B7} and —C(═O)OR^{B8}; R^{B5a1} can be selected from hydrogen or deuterium; R^{B5b1} can be selected from hydrogen, an optionally substituted C₁₋₆ alkyl, an optionally substituted C₂₋₆ alkenyl, an optionally substituted C₃₋₆ cycloalkyl, —C(═O)R^{B9} and —C(═O)OR^{B10}; R^{B6b} can be selected from hydrogen, deuterium, halogen, —C≡N, —C(═O)NH₂, an optionally substituted C₁₋₆ alkyl, an optionally substituted C₂₋₆ alkenyl and an optionally substituted C₂₋₆ alkynyl; R^{B7}, R^{B8}, R^{B9} and R^{B10} can independently be selected from an optionally substituted C₁₋₆ alkyl, an optionally substituted C₂₋₆ alkenyl, an optionally substituted C₂₋₆ alkynyl, an optionally substituted C₃₋₆ cycloalkyl, an optionally substituted C₅₋₁₀ cycloalkenyl, an optionally substituted C₆₋₁₀ aryl, an optionally substituted heteroaryl, an optionally substituted heterocyclyl, an optionally substituted aryl (C₁₋₆ alkyl), an optionally substituted heteroaryl (C₁₋₆ alkyl) and an optionally substituted heterocyclyl (C₁₋₆ alkyl); R^{1A} can be hydrogen, an optionally substituted acyl, an optionally substituted O-linked amino acid or

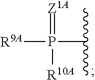

R^{2A}, R^{3A}, R^{5A} and R^{A} can independently be hydrogen or deuterium; R^{4A} can be hydrogen, deuterium or fluoro R^{6A} can be selected from —OH, —OC(═O)R″^{A} and an optionally substituted O-linked amino acid; R^{7A} can be —OH, —OC(═O)R″^{B}, fluoro or chloro; R^{8A} can be an optionally substituted C₁₋₃ alkyl, an optionally substituted C₂₋₆ alkenyl or an optionally substituted C₂₋₆ alkynyl; R^{9A} and R^{10A} can independently be selected from O⁻, —OH, an optionally substituted —O—C₁₋₂₄ alkyl, an optionally substituted —O—C₂₋₂₄ alkenyl, an optionally substituted —O—C₂₋₂₄ alkynyl, an optionally substituted —O—C₃₋₆ cycloalkyl, an optionally substituted —O—C₅₋₁₀ cycloalkenyl, an optionally substituted —O-aryl, an optionally substituted —O-heteroaryl, an optionally substituted —O-aryl (C₁₋₆ alkyl), an optionally substituted *—O—(CR^{11A}R^{12A})ₚ—O—C₁₋₂₄ alkyl, an optionally substituted *—O—(CR^{13A}R^{14A})_{q}—O—C₂₋₂₄ alkenyl,

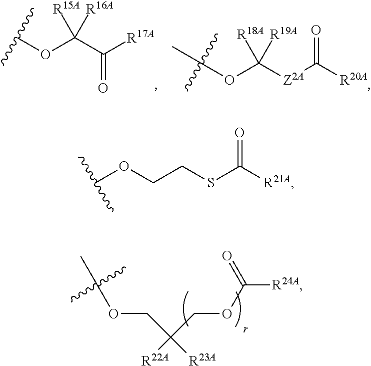

an optionally substituted N-linked amino acid or an optionally substituted N-linked amino acid ester derivative; or R^{9A} can be

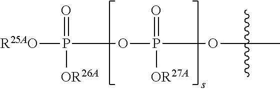

and R^{10A} can be O⁻ or OH; or R^{9A} and R^{10A} can be taken together to form a moiety selected from an optionally substituted

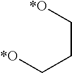

and an optionally substituted

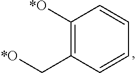

wherein the phosphorus and the moiety form a six-membered to ten-membered ring system; each R^{11A}, each R^{12A}, each R^{13A} and each R^{14A} can be independently hydrogen, deuterium, an optionally substituted C₁₋₂₄ alkyl or alkoxy; R^{15A}, R^{16A}, R^{18A} and R^{19A} can be independently selected from hydrogen, deuterium, an optionally substituted C₁₋₂₄ alkyl and an optionally substituted aryl; R^{17A} and R^{20A} can be independently selected from hydrogen, deuterium, an optionally substituted C₁₋₂₄ alkyl, an optionally substituted aryl, an optionally substituted —O—C₁₋₂₄ alkyl, an optionally substituted —O-aryl, an optionally substituted —O-heteroaryl and an optionally substituted —O-monocyclic heterocyclyl; R^{12A} can be selected from hydrogen, deuterium, an optionally substituted C₁₋₂₄ alkyl and an optionally substituted aryl; R^{22A} and R^{23A} can be independently selected from —C≡N, an optionally substituted C₂₋₈ organylcarbonyl, an optionally substituted C₂₋₈ alkoxycarbonyl and an optionally substituted C₂₋₈ organylaminocarbonyl; R^{24A} can be selected from hydrogen, deuterium, an optionally substituted C₁₋₂₄-alkyl, an optionally substituted C₂₋₂₄ alkenyl, an optionally substituted C₂₋₂₄ alkynyl, an optionally substituted C₃₋₆ cycloalkyl and an optionally substituted C₅₋₁₀ cycloalkenyl; R^{25A}, R^{26A} and R^{27A} can be independently absent, hydrogen or deuterium; p and q can be independently selected from 1, 2 and 3; r can be 1 or 2; s can be 0 or 1; R″^{A} and R″^{B} can be independently an optionally substituted C₁₋₂₄ alkyl; and Z^{1A} and Z^{2A} can be independently oxygen (O) or sulfur (S). In this paragraph, the asterisks indicate the points of attachment of the moieties.

In some embodiments, a compound of Formula (I), or a pharmaceutically acceptable salt thereof can be wherein: B^{1A} can be,

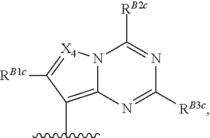

wherein, X⁴ can be N (nitrogen) or —CR^{B6c}; R^{B1c} can be hydrogen or deuterium; R^{B2c} can be NR^{B4a2}R^{B4b2}; R^{B3c} can be selected from hydrogen, deuterium, halogen or NR^{B5a2}R^{B5b2}; R^{B4a2} can be hydrogen or deuterium; R^{B4b2} can be selected from hydrogen, deuterium, an optionally substituted C₁₋₆ alkyl, an optionally substituted C₂₋₆ alkenyl, an optionally substituted C₃₋₆ cycloalkyl, —C(═O)R^{B7} and —C(═O)OR^{B8}; R^{B5a2} can be selected from hydrogen or deuterium; R^{B5b2} can be selected from hydrogen, an optionally substituted C₁₋₆ alkyl, an optionally substituted C₂₋₆ alkenyl, an optionally substituted C₃₋₆ cycloalkyl, —C(═O)R^{B9} and C(═O)OR^{B10}; R^{B6c} can be selected from hydrogen, deuterium, halogen, —C≡N, —C(═O)NH₂, an optionally substituted C₁₋₆ alkyl, an optionally substituted C₂₋₆ alkenyl and an optionally substituted C₂₋₆ alkynyl; R^{B7}, R^{B8}, R^{B9} and R^{B10} can independently be selected from an optionally substituted C₁₋₆ alkyl, an optionally substituted C₂₋₆ alkenyl, an optionally substituted C₂₋₆ alkynyl, an optionally substituted C₃₋₆ cycloalkyl, an optionally substituted C₅₋₁₀ cycloalkenyl, an optionally substituted C₆₋₁₀ aryl, an optionally substituted heteroaryl, an optionally substituted heterocyclyl, an optionally substituted aryl (C₁₋₆ alkyl), an optionally substituted heteroaryl (C₁₋₆ alkyl) and an optionally substituted heterocyclyl (C₁₋₆ alkyl); R^{1A} can be hydrogen, an optionally substituted acyl, an optionally substituted O-linked amino acid of

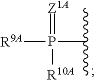

R^{2A}, R^{3A}, R^{5A} and R^{A} can independently be hydrogen or deuterium; R^{4A} can be hydrogen, deuterium or fluoro; R^{6A} can be selected from —OH, —OC(═O)R″^{A} and an optionally substituted O-linked amino acid; R^{7A} can be —OH, —OC(═O)R″^{B}, fluoro or chloro; R^{8A} can be an optionally substituted C₁₋₃ alkyl, an optionally substituted C₂₋₆ allenyl or an optionally substituted C₂₋₆ alkynyl; R^{9A} and R^{10A} can independently be selected from O⁻, —OH, an optionally substituted —O—C₁₋₂₄ alkyl, an optionally substituted —O—C₂₋₂₄ alkenyl, an optionally substituted —O—C₂₋₂₄ alkynyl, an optionally substituted —O—C₃₋₆ cycloalkyl, an optionally substituted —O—C₅₋₁₀ cycloalkenyl, an optionally substituted —O-aryl, an optionally substituted —O-heteroaryl, an optionally substituted —O-aryl (C₁₋₆ alkyl), an optionally substituted *—O—(CR^{11A}R^{12A})ₚ—O—C₁₋₂₄ alkyl, an optionally substituted *—O—(C^{13A}R^{14A})_{q}—O—C₂₋₂₄ alkenyl,

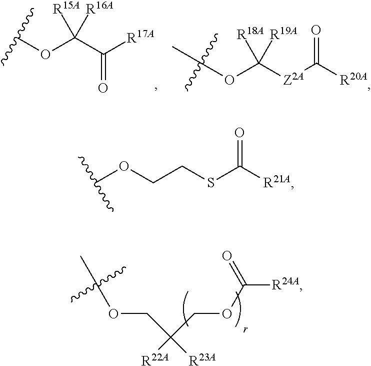

an optionally substituted N-linked amino acid or an optionally substituted N-linked amino acid ester derivative; or R^{9A} can be

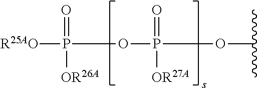

and R^{10A} can be O⁻ or OH; or R^{9A} and R^{10A} can be taken together to form a moiety selected from an optionally substituted

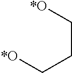

and an optionally substituted

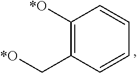

wherein the phosphorus and the moiety form a six-membered to ten-membered ring system; each R^{11A}, each R^{12A}, each R^{13A} and each R^{14A} can be independently hydrogen, deuterium, an optionally substituted C₁₋₂₄ alkyl or alkoxy; R^{15A}, R^{16A}, R^{18A} and R^{19A} can be independently selected from hydrogen, deuterium, an optionally substituted C₁₋₂₄ alkyl and an optionally substituted aryl; R^{17A} and R^{20A} can be independently selected from hydrogen, deuterium, an optionally substituted C₁₋₂₄ alkyl, an optionally substituted aryl, an optionally substituted —O—C₁₋₂₄ alkyl, an optionally substituted —O-aryl, an optionally substituted —O-heteroaryl and an optionally substituted —O-monocyclic heterocyclyl; R^{21A} can be selected from hydrogen, deuterium, an optionally substituted C₁₋₂₄ alkyl and an optionally substituted aryl; R^{22A} and R^{23A} can be independently selected from —C≡N, an optionally substituted C₂₋₈ organylcarbonyl, an optionally substituted C₂₋₈ alkoxycarbonyl and an optionally substituted C₂₋₈ organylaminocarbonyl; R^{24A} can be selected from hydrogen, deuterium, an optionally substituted C₁₋₂₄-alkyl, an optionally substituted C₂₋₂₄ alkenyl, an optionally substituted C₂₋₂₄ alkynyl, an optionally substituted C₃₋₆ cycloalkyl and an optionally substituted C₅₋₁₀ cycloalkenyl; R^{25A}, R^{26A} and R^{27A} can be independently absent, hydrogen or deuterium; p and q can be independently selected from 1, 2 and 3; r can be 1 or 2; s can be 0 or 1; R″^{A} and R″^{B} can be independently an optionally substituted C₁₋₂₄ alkyl; and Z^{1A} and Z^{2A} can be independently oxygen (O) or sulfur (S). In this paragraph, the asterisks indicate the points of attachment of the moieties.

In some embodiments, a compound of Formula (I), or a pharmaceutically acceptable salt thereof can be wherein: B^{1A} can be,

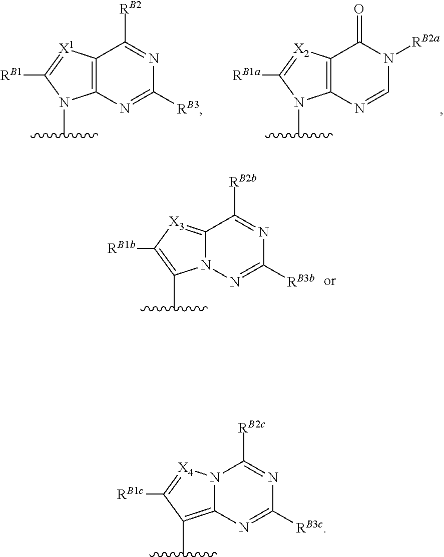

In some embodiments, X¹ can be N (nitrogen) or —CR^{B6}; X² can be N (nitrogen) or —CR^{B6a}; X³ can be N (nitrogen) or —CR^{B6b}; X⁴ can be N (nitrogen) or —CR^{B6c}; R^{B1}, R^{B1a}, R^{B1b} and R^{B1c} can independently be selected from hydrogen or deuterium; R^{B2} can be NR^{B4a}R^{B4b}; R^{B2b} can be NR^{B4a1}R^{B4b1}; R^{B2c} can be NR^{B4a1}R^{B4b1}; R^{B2a} can be hydrogen, an optionally substituted C₁₋₆ alkyl, an optionally substituted C₂₋₆ alkenyl and an optionally substituted C₃₋₆ cycloalkyl; R^{B3} can be selected from hydrogen, deuterium, halogen or NR^{B5a}R^{B5b}; R^{B3b} can be selected from hydrogen, deuterium, halogen or NR^{B5a1}R^{B5b1}; R^{B3c} can be selected from hydrogen, deuterium, halogen or NR^{B5a2}R^{B5b2}; R^{B4a}, R^{B4a1} and R^{B4a2} can be independently hydrogen or deuterium; R^{B4b}, R^{B4b1} and R^{B4b2} can be independently selected from hydrogen, deuterium, an optionally substituted C₁₋₆ alkyl, an optionally substituted C₂₋₆ alkenyl, an optionally substituted C₃₋₆ cycloalkyl, —C(═O)R^{B7} and —C(═O)OR^{B8}; R^{B5a} can be selected from hydrogen or deuterium; R^{B5b} can be selected from hydrogen, an optionally substituted C₁₋₆ alkyl, an optionally substituted C₂₋₆ alkenyl, an optionally substituted C₃₋₆ cycloalkyl, C(═O)R^{B9} and —C(═O)OR^{B10}; R^{B6}, R^{B6a}, R^{B6b} and R^{B6c} can independently be selected from hydrogen, deuterium, halogen, —C≡N, —C(═O)NH₂; an optionally substituted C₁₋₆ alkyl, an optionally substituted C₂₋₆ alkenyl and an optionally substituted C₂₋₆ alkynyl; R^{B7}, R^{B8}, R^{B9} and R^{B10} can independently be selected from an optionally substituted C₁₋₆ alkyl, an optionally substituted C₂₋₆ alkenyl, an optionally substituted C₂₋₆ alkynyl, an optionally substituted C₃₋₆ cycloalkyl, an optionally substituted C₅₋₁₀ cycloalkenyl, an optionally substituted C₆₋₁₀ aryl, an optionally substituted heteroaryl, an optionally substituted heterocyclyl, an optionally substituted aryl (C₁₋₆ alkyl), an optionally substituted heteroaryl (C₁₋₆ alkyl) and an optionally substituted heterocyclyl (C₁₋₆ alkyl); R^{1A} can be hydrogen, an optionally substituted acyl, an optionally substituted O-linked amino acid or

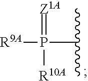

R^{5A} can be hydrogen or deuterium; R^{4A} can be hydrogen, deuterium or fluoro; R^{6A} can be selected from —OH and —OC(═O)R″^{A}; R^{7A} can be —OH, —OC(═O)R″^{B} or fluoro; R^{9A} and R^{10A} can independently be selected from O⁻, —OH, an optionally substituted —O—C₁₋₂₄ alkyl, an optionally substituted —O—C₂₋₂₄ alkenyl, an optionally substituted −O—C₂₋₂₄ alkynyl, an optionally substituted —O—C₃₋₆ cycloalkyl, an optionally substituted —O—C₅₋₁₀ cycloalkenyl, an optionally substituted —O-aryl, alkenyl, an optionally substituted N-linked amino acid or an optionally substituted N-linked amino acid ester derivative; or R^{9A} can be

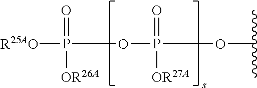

and R^{10A} can be O⁻ or OH; R^{25A}, R^{26A} and R^{27A} can be independently absent, hydrogen or deuterium; s can be 0 or 1; R″^{A} and R″^{B} can be independently an optionally substituted C₁₋₂₄ alkyl; and Z^{1A} and Z^{2A} can be independently oxygen (O) or sulfur (S).

In some embodiments, a compound of Formula (I), or a pharmaceutically acceptable salt thereof can be B^{1A} can be

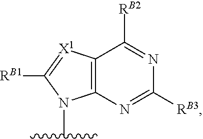

wherein: X¹ can be N (nitrogen) or —CR^{B6}; R^{B1} can be hydrogen or deuterium; R^{B2} can be NR^{B4a}R^{B4b}; R^{B4b}, R^{B3} can be hydrogen, deuterium, halogen or NR^{B5a}R^{B5b}; R^{B4a} can be hydrogen or deuterium, R^{B4b} can be selected from hydrogen, an optionally substituted C₁₋₆ alkyl, an optionally substituted C₃₋₆ alkenyl, an optionally substituted C₃₋₆ cycloalkyl, —C(═O)R^{B7} and —C(═O)OR^{B8}; R^{B5a} can be hydrogen or deuterium; R^{B5a} can be selected from hydrogen, an optionally substituted C₁₋₆ alkyl, an optionally substituted C₃₋₆ alkenyl, an optionally substituted C₃₋₆ cycloalkyl, C(═O)R^{B9} and —C(═O)OR^{B10}; R^{B6} can be selected from hydrogen, deuterium, halogen, —C(═O)NH₂, an optionally substituted C₁₋₆ alkyl, an optionally substituted C₂₋₆ alkenyl and an optionally substituted C₂₋₆ alkynyl; R^{B7}, R^{B8}, R^{B9} and R^{B10} can be independently selected from C₁₋₆ alkyl, C₂₋₆ alkenyl, C₂₋₆ alkynyl, cycloalkyl, C₃₋₆ cycloalkenyl, C₆₋₁₀ aryl, heteroaryl, heterocyclyl, aryl (C₁₋₆ alkyl), heteroaryl (C₁₋₆ alkyl) and heterocyclyl (C₁₋₆ alkyl); R^{1A} can be hydrogen, an optionally substituted acyl, an optionally substituted O-linked amino acid or

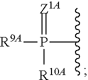

R^{2A}, R^{3A}, R^{5A} and R^{A} can be independently hydrogen or deuterium; R^{4A} can be hydrogen, deuterium or fluoro; R^{6A} can be selected from —OH, —OC(═O)R″^{A} and an optionally substituted O-linked amino acid; R^{7A} can be —OH, fluoro or chloro; R^{8A} can be an optionally substituted C₁₋₃ alkyl, an optionally substituted C₂₋₆ allenyl or an optionally substituted C₂₋₆ alkynyl; R^{9A} and R^{10A} can be independently selected from O⁻, —OH, an optionally substituted —O—C₁₋₂₄ alkyl, an optionally substituted —O—C₂₋₂₄ alkenyl, an optionally substituted —O—C₂₋₂₄ alkynyl, an optionally substituted —O—C₃₋₆ cycloalkyl, an optionally substituted —O—C₃₋₆ cycloalkenyl, an optionally substituted —O-aryl, an optionally substituted —O-heteroaryl, an optionally substituted —O-aryl (C₁₋₆ alkyl), an optionally substituted *—O—(CR^{11A}R^{12A})ₚ—O—C₁₋₂₄ alkyl, an optionally substituted *—O—(CR^{13A}R^{14A})_{q}—O—C₁₋₂₄ alkenyl,

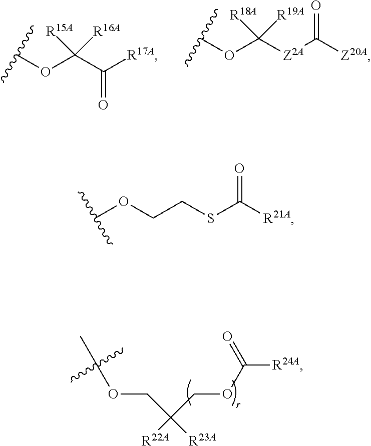

an optionally substituted N-linked amino acid or an optionally substituted N-linked amino acid ester derivative; or R^{9A} can be

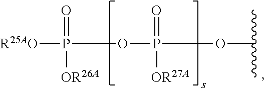

and R^{10A} can be O⁻ or OH; or R^{9A} and R^{10A} can be taken together to form a moiety selected from an optionally substituted

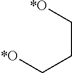

and an optionally substituted

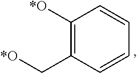

wherein the phosphorus and the moiety form a six-membered to ten-membered ring system; each R^{11A}, each R^{12A}, each R^{13A} and each R^{14A} can be independently hydrogen, deuterium, an optionally substituted C₁₋₂₄ alkyl or alkoxy; R^{15A}, R^{16A}, R^{18A} and R^{19A} can be independently selected from hydrogen, deuterium, an optionally substituted C₁₋₂₄ alkyl and an optionally substituted aryl; R^{17A} and R^{20A} can be independently selected from hydrogen, deuterium, an optionally substituted C₁₋₂₄ alkyl, an optionally substituted aryl, an optionally substituted —O—C₁₋₂₄ alkyl, an optionally substituted —O-aryl, an optionally substituted —O-heteroaryl, an optionally substituted —O-monocyclic heterocyclyl; R^{21A} can be selected from hydrogen, deuterium, an optionally substituted C₁₋₂₄ alkyl and an optionally substituted aryl; R^{22A} and R^{23A} can be independently selected from —C≡N, an optionally substituted C₂₋₅ organylcarbonyl, an optionally substituted C₂₋₈ alkoxycarbonyl and an optionally substituted C₂₋₈ organylaminocarbonyl; R^{24A} can be selected from hydrogen, deuterium, an optionally substituted C₁₋₂₄-alkyl, an optionally substituted C₂₋₂₄ alkenyl, an optionally substituted C₂₋₂₄ alkynyl, an optionally substituted C₃₋₆ cycloalkyl and an optionally substituted C₃₋₆ cycloalkenyl; R^{25A}, R^{26A} and R^{27A} can be independently absent, hydrogen or deuterium; p and q can be independently selected from 1, 2 and 3; r can be 1 or 2; s can be 0 or 1; R″^{A} can be an optionally substituted C₁₋₂₄ alkyl; and Z^{1A} and Z^{2A} can be independently oxygen (O) or sulfur (S); and provided that When X¹ is N or CH, then (a) R^{4A} is fluoro, (b) R^{B3} is halogen or NR^{B5a}R^{B5b}, (c) R^{8A} is optionally substituted C₂₋₆ allenyl, or (d) any two or all three of said (a), (b) and (c) are present; and provided that when X¹ is N or CH, R^{4A} is fluoro and R^{1A} is hydrogen or triphosphate, then R^{8A} is not methyl; and provided that the compound of Formula (I) is not selected from the group consisting of

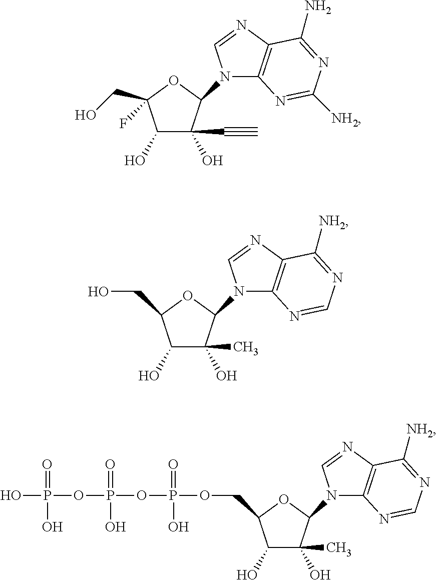 [SMILES: C#C[C@]1(O)[C@H](n2cnc3c(N)nc(N)nc32)O[C@](F)(CO)[C@H]1O ; C[C@@]1(O)[C@H](O)[C@@H](CO)O[C@H]1n1cnc2c(N)ncnc21 ; C[C@@]1(O)[C@H](O)[C@@H](COP(=O)(O)OP(=O)(O)OP(=O)(O)O)O[C@H]1n1cnc2c(N)ncnc21]

and a pharmaceutically acceptable salt thereof.

Other embodiments disclosed herein relate to a compound of Formula (II), or a pharmaceutically acceptable salt thereof:

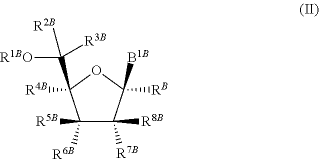

wherein: B^{1B} can be

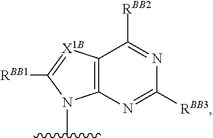

wherein: X^{1B} can be N (nitrogen) or —CR^{BB6}; R^{BB1} can be hydrogen or deuterium, R^{BB2} can be NR^{BB4a}R^{BB4b}; R^{BB3} can be halogen or NR^{BB5a}R^{BB5b}; R^{BB4a} can be hydrogen or deuterium; R^{BB4b} can be selected from hydrogen, deuterium, an optionally substituted C₁₋₆ alkyl, an optionally substituted C₃₋₆ alkenyl, an optionally substituted C₃₋₆ cycloalkyl, —C(═O)R^{BB7} and —C(═O)OR^{BB8}; R^{BB5a} can be hydrogen or deuterium; R^{BB5b} can be selected from hydrogen, deuterium, an optionally substituted C₁₋₆ alkyl, an optionally substituted C₃₋₆ alkenyl, an optionally substituted C₃₋₆ cycloalkyl, —C(═O)R^{BB9} and —C(═O)OR^{BB10}; R^{BB6} can be selected from hydrogen, deuterium. halogen, —C≡N, an optionally substituted C₁₋₆ alkyl, an optionally substituted C₂₋₆ alkenyl, an optionally substituted C₂₋₆ alkynyl or —C(═O)NH₂; R^{BB7}, R^{BB8}, R^{BB9} and R^{BB10} can be independently selected from C₁₋₆ alkyl, C₂₋₆ alkenyl, C₂₋₆ alkynyl, C₃₋₆ cycloalkyl, C₅₋₁₀ cycloalkenyl, C₆₋₁₀ aryl, heteroaryl, heterocyclyl, aryl (C₁₋₆ alkyl), heteroaryl (C₁₋₆ alkyl) and heterocyclyl (C₁₋₆ alkyl); R^{1B} can be hydrogen, deuterium, an optionally substituted acyl, an optionally substituted O-linked amino acid or

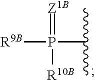

R^{2B}, R^{3B}, R^{5B} and R^{B} can be independently hydrogen or deuterium; R^{4B} can be fluoro; R^{6B} can be selected from —OH, —OC(═O)R″^{B} and an optionally substituted O-linked amino acid; R^{7B} can be —OH, fluoro or chloro; R^{8B} can be an unsubstituted C₂₋₆ alkenyl or an unsubstituted C₂₋₆ alkynyl; R^{9B} and R^{10B} can be independently selected from O⁻, —OH, an optionally substituted —O—C₁₋₂₄ alkyl, an optionally substituted —O—C₂₋₂₄ alkenyl, an optionally substituted —O—C₂₋₂₄ alkynyl, an optionally substituted —O—C₃₋₆ cycloalkyl, an optionally substituted —O—C₅₋₁₀ cycloalkenyl, an optionally substituted an optionally substituted —O-heteroaryl, an optionally substituted —O-heteroaryl, an optionally substituted —O-aryl (C₁₋₆ alkyl), an optionally substituted *—O—(CR^{11B}R^{12B})ₜ—O—C₁₋₂₄ alkyl, an optionally substituted *—O—(CR^{13B}R^{14B})ᵤ—O—C₁₋₂₄ alkenyl,

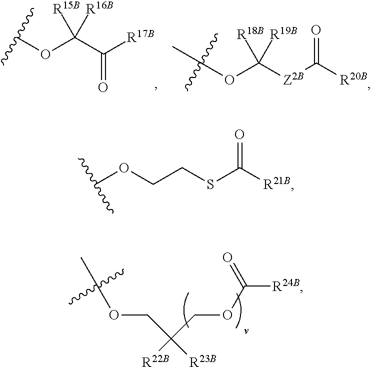

an optionally substituted N-linked amino acid or an optionally substituted N-linked amino acid ester derivative; or R^{9B} can b

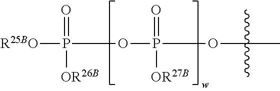

(and R^{10B} is O⁻ or OH; or R^{9B} and R^{10B} can be taken toaether to form a moiety selected from an optionally substituted

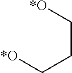

and an optionally substituted

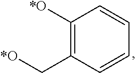

wherein the phosphorus and the moiety form a six-membered to ten-membered ring system; each R^{11B} each R^{12B} , each R^{13B} and each R^{14B} can be independently hydrogen, deuterium, an optionally substituted C₁₋₂₄ alkyl or alkoxy; R^{15B}, R^{16B}, R^{18B} and R^{19B} can be independently selected from hydrogen, deuterium, an optionally substituted C₁₋₂₄ alkyl and an optionally substituted aryl; R^{17B} and R^{20B} can be independently selected from hydrogen, deuterium, an optionally substituted C₁₋₂₄ alkyl, an optionally substituted aryl, an optionally substituted —O—C₁₋₂₄ alkyl, an optionally substituted —O-aryl, an optionally substituted —O-heteroaryl, an optionally substituted —O-monocyclic heterocyclyl; R^{21B} can be selected from hydrogen, deuterium, an optionally substituted C₁₋₂₄ alkyl and an optionally substituted aryl; R^{22B} and R^{23B} can be independently selected from —C≡N, an optionally substituted C₂₋₈ organylcarbonyl, an optionally substituted C₂₋₈ alkoxycarbonyl and an optionally substituted C₂₋₈ organylaminocarbonyl; R^{24B} can be selected from hydrogen, deuterium, an optionally substituted C₁₋₂₄-alkyl, an optionally substituted C₂₋₂₄ alkenyl, an optionally substituted C₂₋₂₄ alkynyl, an optionally substituted C₃₋₆ cycloalkyl and an optionally substituted C₅₋₁₀ cycloalkenyl; R^{25B}, R^{26B} and R^{27B} can be independently absent or hydrogen, deuterium; t and u can be independently selected from 1, 2 and 3; v can be 1 or 2; w can be 0 or 1; R″^{B} can be an optionally substituted C₁₋₂₄ alkyl; and Z^{1B} and Z^{2B} can be independently oxygen (O) or sulfur (S). In this paragraph, the asterisks indicate the points of attachment of the moieties.

In some embodiments, R^{1B} can be hydrogen or deuterium. In some embodiments, R^{1B} can be an optionally substituted acyl. In other embodiments, R^{1B} can be —C(═O)R″^{B1}, wherein R″^{B1} can be an optionally substituted C₁₋₁₂ alkyl. In some embodiments, R″^{B1} can be an unsubstituted C₁₋₄ alkyl.

In still other embodiments, R^{1B} can be an optionally substituted O-linked amino acid, for example, an optionally substituted O-linked α-amino acid. Examples of suitable O-linked amino acids include alanine, asparagine, aspartate, cysteine, glutamate, glutamine, glycine, proline, serine, tyrosine, arginine, histidine, isoleucine, leucine, lysine, methionine, phenylalanine, threonine, tryptophan and valine. Additional examples of suitable amino acids include, but are not limited to, ornithine, hypusine, 2-aminoisobutyric acid, dehydroalanine, gamma-aminobutyric acid, citrulline, beta-alanine, alpha-ethyl-glycine, alpha-propyl-glycine and norleucine. In some embodiments, the O-linked amino acid can have the structure

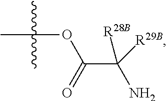

wherein R^{28B} can be selected from hydrogen, deuterium, an optionally substituted C₁₋₆ alkyl, an optionally substituted C₁₋₆ haloalkyl, an optionally substituted C₃₋₆ cycloalkyl, an optionally substituted C₆ aryl, an optionally substituted C₁₀ aryl and an optionally substituted aryl (C₁₋₆ alkyl); and R^{29B} can be hydrogen, deuterium or an optionally substituted C₁₋₄-alkyl; or R^{28B} and R^{29B} can be taken together to form an optionally substituted C₃₋₆ cycloalkyl. Those skilled in the art understand that when R^{1B} is an optionally substituted O-linked amino acid, the oxygen of R^{1B}O— of Formula (II) is part of the optionally substituted O-linked amino acid. For example, when R^{1B} is

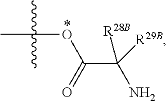

the oxygen indicated with “*” is the oxygen of R^{1B}O— of Formula (II).

When R^{28B} is substituted, R^{28B} can be substituted with one or more substituents selected from N-amino, mercapto, alkylthio, an optionally substituted aryl, hydroxy, an optionally substituted heteroaryl, O-carboxy and amino. In some embodiments, R^{28B} can be an unsubstituted C₁₋₆-alkyl, such as those described herein. In some embodiments, R^{28B} can be hydrogen or deuterium. In other embodiments, R^{28B} can be methyl. In some embodiments, R^{29B} can be hydrogen or deuterium. In other embodiments, R^{29B} can be an optionally substituted C₁₋₄-alkyl, such as methyl, ethyl, n-propyl, isopropyl, n-butyl, isobutyl and tert-butyl. In an embodiment, R^{29B} can be methyl. Depending on the groups that are selected for R^{28B} and R^{29B}, the carbon to which R^{28B} and R^{29B} are attached may be a chiral center. In some embodiment, the carbon to which R^{28B} and R^{29B} are attached may be a (R)-chiral center. In other embodiments, the carbon to which R^{28B} and. R^{29B} are attached may be a (S)-chiral center. In this paragraph, the asterisks indicate the points of attachment of the moieties.

Examples of suitable

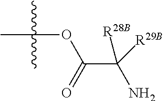

include the following:

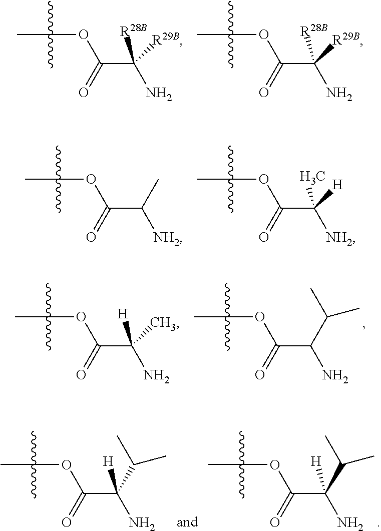

In some embodiments, R^{1B} can be

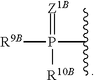

A variety of R^{9B} and R^{10B} groups can be attached to the phosphorus atom of Formula (II). In some embodiments, R^{9B} and R^{10B} can be both —OH. In other embodiments, R^{9B} and R^{10B} can be both O⁻. In still other embodiments, at least one R^{9B} and R^{10B} can be absent. In yet still other embodiments, at least one R^{9B} and R^{10B} can be hydrogen or deuterium. Those skilled in the art understand that when R^{9B} and/or R^{10B} are absent, the associated oxygen(s) will have a negative charge. For example, when R^{9B} is absent, the oxygen associated with R^{9B} will have a negative charge. In some embodiments, Z^{1B} can be O (oxygen). In other embodiments, Z^{1B} can be S (sulfur). In some embodiments, R^{1B} can be a monophosphate. In other embodiments, R^{1B} can be a monothiophosphate.

In some embodiments, one of R^{9B} and R^{10B} can be O⁻ or —OH and the other of R^{9B} and R^{10B} can be selected from an optionally substituted —O—C₁₋₂₄ alkyl, an optionally substituted —O—C₂₋₂₄ alkenyl, an optionally substituted —O—C₂₋₂₄ alkynyl, an optionally substituted —O—C₃₋₆ cycloalkyl, an optionally substituted —O—C₅₋₁₀ cycloalkenyl, an optionally substituted —O-aryl, an optionally substituted —O-heteroaryl and an optionally substituted —O-aryl (C₁₋₆ alkyl). In some embodiments, one of R^{9B} and R^{10B} can be O⁻ or —OH and the other of R^{9B} and R^{10B} can be an optionally substituted —O—C₁₋₂₄ alkyl. In other embodiments, both R^{9B} and R^{10B} can be independently selected from an optionally substituted —O—C₁₋₂₄ alkyl, an optionally substituted —O—C₂₋₂₄ alkenyl, an optionally substituted —O—C₂₋₂₄ alkynyl, an optionally substituted —O—C₃₋₆ cycloalkyl, an optionally substituted —O—C₅₋₁₀ cycloalkenyl, an optionally substituted —O-aryl, an optionally substituted —O-heteroaryl and an optionally substituted —O-aryl (C₁₋₆ alkyl). In some embodiments, both R^{9B} and R^{10B} can be an optionally substituted —O—C₁₋₂₄ alkyl. In other embodiments, both R^{9B} and R^{10B} can be an optionally substituted —O—C₂₋₂₄ alkenyl. In some embodiments, R^{9B} and R^{10B} can be independently an optionally substituted group selected from the following: —O-myristoleyl, —O-myristyl, —O-palmitoleyl, —O-palmityl, —O-sapienyl, —O-oleyl, —O-elaidyl, —O-vaccenyl, —O-linoleyl, —O-α-inolenyl, —O-arachidonyl, —O-eicosapentaenyl, —O-erucyl, —O-docosahexaenyl, —O-capryl, —O-lauryl, —O-stearyl, —O-arachidyl, —O-behenyl, —O-lignoceryl and —O-cerotyl.

In some embodiments, at least one of R^{9B} and R^{9B} can be an optionally substituted *—O—(CR^{11B}R^{12B})ₜ—O—C₁₋₂₄ alkyl. In other embodiments, R^{9B} and R^{10B} can be both an optionally substituted *—O—(CR^{11B}R^{12B})ₜ—O—C₁₋₂₄ alkyl. In some embodiments, each R^{11B} and each R^{12B} can be hydrogen or deuterium. In other embodiments, at least one of R^{11B} and R^{12B} can be an optionally substituted C₁₋₂₄ alkyl. In other embodiments, at least one of R^{11B} and R^{12B} can be an alkoxy (for example, benzoxy). In some embodiments, t can be 1. In other embodiments, t can be 2. In still other embodiments, t can be 3.

In some embodiments, at least one of R^{9B} and R^{10B} can be an optionally substituted *—O—(CR^{13B}R^{14B})ᵤ—O—C₁₋₂₄ alkenyl. In other embodiments, R^{9B} and R^{10B} can be both an optionally substituted *—O—(CR^{13B}R^{14B})ᵤ—O—C₁₋₂₄ alkenyl. In some embodiments, each R^{13B} and each R^{14B} can be hydrogen or deuterium. In other embodiments, at least one of R^{13B} and R^{14B} can be an optionally substituted C₁₋₂₄ alkyl. In some embodiments, u can be 1. In other embodiments, u can be 2. In still other embodiments, u can be 3. When at least one of R^{9B} and R^{10B} is *—O—(CR^{11B}R^{12B})ₜ—O—C₁₋₂₄ alkyl or an optionally substituted *—O—(CR^{13B}R^{14B})ᵤ—O—C₁₋₂₄ alkenyl, the C₁₋₂₄ alkyl can be selected from caprylyl, capryl, lauryl, myristyl, palmityl, stearyl, arachidyl, behenyl, lignoceryl and cerotyl, and the C₂₋₂₄ alkenyl can be selected from myristoleyl, palmitoleyl, sapienyl, oleyl, elaidyl, vaccenyl, linoleyl, α-linolenyl, arachidonyl, eicosapentaenyl, erucyl and docosahexaenyl.

In some embodiments, at least one of R^{9B} and R^{10B} can be selected from

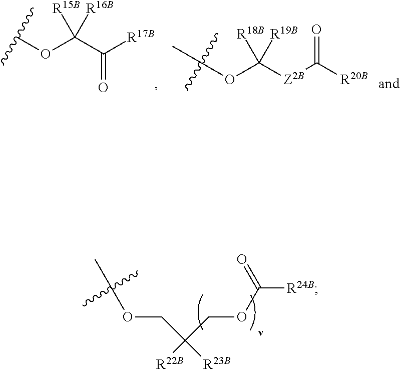

and the other of R^{9B} and R^{10B} can be selected from O⁻, —OH, an optionally substituted —O—C₁₋₂₄ alkyl, an optionally substituted —O—C₂₋₂₄ alkenyl, an optionally substituted —O—C₂₋₂₄ alkynyl, an optionally substituted —O—C₃₋₆ cycloalkyl, an optionally substituted —O—C₅₋₁₀ cycloalkenyl, an optionally substituted —O-aryl, an optionally substituted —O-heteroaryl and an optionally substituted —O-aryl (C₁₋₆ alkyl).

In some embodiments, at least one of R^{9B} and R^{10B} can be

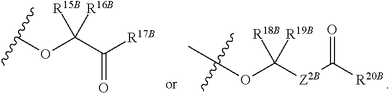

In some embodiments, both R^{9B} and R^{10B} can be

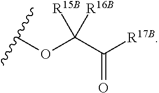

When one or both of R^{9B} and R^{10B} are

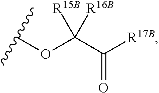

R^{15B} and R^{16B} can be independently selected from hydrogen, deuterium, an optionally substituted C₁₋₂₄ alkyl and an optionally substituted aryl; and R^{17B} can be selected from hydrogen, deuterium, an optionally substituted C₁₋₂₄ alkyl, an optionally substituted aryl, an optionally substituted —O—C₁₋₂₄ alkyl, an optionally substituted —O-aryl, an optionally substituted —O-heteroaryl and an optionally substituted —O-monocyclic heterocyclyl. In some embodiments, R^{15B} and R^{16B} can be hydrogen or deuterium. In other embodiments, at least one of R^{15B} and R^{16B} can be an optionally substituted C₁₋₂₄ alkyl or an optionally substituted aryl. In some embodiments, R^{17B} can be an optionally substituted C₁₋₂₄ alkyl. In some embodiments, R^{17B} can be an unsubstituted C₁₋₄ alkyl. In other embodiments, R^{17B} can be an optionally substituted aryl. In still other embodiments, R^{17B} can be an optionally substituted —O—C₁₋₂₄ alkyl, an optionally substituted —O-aryl, an optionally substituted —O-heteroaryl or an optionally substituted —O-monocyclic heterocyclyl. In some embodiments, R^{17B} can be an unsubstituted —O—C₁₋₄ alkyl.

In some embodiments, both R^{9B} and R^{10B} can be

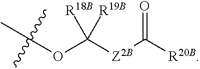

When one or both of R^{9B} and R^{10B} are

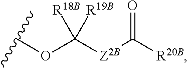

R^{18B} and R^{19B} can be independently selected from hydrogen, deuterium, an optionally substituted C₁₋₂₄ alkyl and an optionally substituted aryl; R^{20B} can be independently selected from hydrogen, deuterium, an optionally substituted C₁₋₂₄ alkyl, an optionally substituted aryl, an optionally substituted —O—C₁₋₂₄ alkyl, an optionally substituted —O-aryl, an optionally substituted —O-heteroaryl and an optionally substituted —O-monocyclic heterocyclyl; and Z^{2B} can be independently O (oxygen) or S (sulfur). In some embodiments, R^{18B} and R^{19B} can be hydrogen or deuterium. In other embodiments, at least one of R^{18B} and R^{19B} can be an optionally substituted C₁₋₂₄ alkyl or an optionally substituted aryl. In some embodiments, R^{20B} can be an optionally substituted C₁₋₂₄ alkyl. In some embodiments, R^{20B} can be an unsubstituted C₁₋₄ alkyl. In other embodiments, R^{20B} can be an optionally substituted aryl. In still other embodiments, R^{20B} can be an optionally substituted —O—C₁₋₂₄ alkyl, an optionally substituted —O-aryl, an optionally substituted —O-heteroaryl or an optionally substituted —O-monocyclic heterocyclyl. In some embodiments, R^{16B} can be an unsubstituted alkyl. In some embodiments, Z^{2B} can be O (oxygen). In other embodiments, Z^{2B} can be or S (sulfur). In some embodiments, one or both of R^{9B} and R^{10B} can be an optionally substituted isopropyloxycarbonyloxymethoxy (POC). In some embodiments, R^{9B} and R^{10B} each can be an optionally substituted isopropyloxycarbonyloxymethoxy (POC) group, and form an optionally substituted bis(isopropyloxycarbonyloxymethyl) (bis(POC)) prodrug. In other embodiments, one or both of R^{9B} and R^{10B} can be an optionally substituted pivaloyloxymethoxy (POM). In some embodiments, R^{9B} and R^{10B} each can be an optionally substituted pivaloyloxymethoxy (POM) group, and form an optionally substituted bis(pivaloyloxymethyl) (bis(POM)) prodrug.

In some embodiments, at least one of R^{9B} and R^{10B} can be

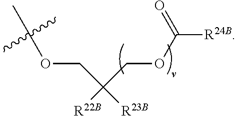

In some embodiments, both R^{9B} and R^{10B} can be

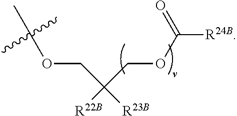

When one or both of R^{9B} and R^{10B} are

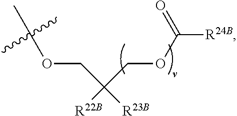

R^{22B} and R^{23B} can be independently —C≡N or an optionally substituted substituent selected from C₂₋₈ organylcarbonyl, C₂₋₈ alkoxycarbonyl and C₂₋₈ organylaminocarbonyl; R^{24B} can be selected from hydrogen, deuterium, an optionally substituted C₁₋₂₄ alkyl, an optionally substituted C₂₋₂₄ alkenyl, an optionally substituted C₂₋₂₄ alkynyl, an optionally substituted C₃₋₆ cycloalkyl and an optionally substituted C₅₋₁₀ cycloalkenyl; and v can be 1 or 2. In some embodiments, R^{22B} can be —C≡N and R^{23B} can be an optionally substituted C₂₋₈ alkoxycarbonyl, such as —C(═O)OCH₃. In other embodiments. R^{22B} can be —C≡N and R^{23B} can be an optionally substituted C₂₋₈ organylaminocarbonyl, for example, —C(═O)NHCH₂CH₃ and —C(═O)NHCH₂CH₂phenyl. In some embodiments, both R^{22B} and R^{23B} can be an optionally substituted C₂₋₈ organylcarbonyl, such as —C(═O)CH₃. In some embodiments, both R^{22B} and R^{23B} can be an optionally substituted C₁₋₈ alkoxycarbonyl, for example, —C(═O)OCH₂CH₃ and —C(═O)OCH₃. In some embodiments, including those described in this paragraph, R^{24B} can be an optionally substituted C₁₋₄ alkyl. In some embodiment, R^{24B} can be methyl or tert-butyl. In some embodiments, v can be 1. In other embodiments, v can be 2.

In some embodiments, R^{9B} and R^{10B} can be both an optionally substituted —O-aryl. In some embodiments, at least one of R^{9B} and R^{10B} can be an optionally substituted —O-aryl. For example, both R^{9B} and R^{10B} can be an optionally substituted —O-phenyl or an optionally substituted —O-naphthyl. When substituted, the substituted —O-aryl can be substituted with 1, 2, 3 or more than 3 substituents. When more than two substituents are present, the substituents can be the same or different. In some embodiments, when at least one of R^{9B} and R^{10B} is a substituted —O-phenyl, the substituted —O-phenyl can be a para, ortho- or meta-substituted.

In some embodiments, R^{9B} and R^{10B} can be both an optionally substituted —O-aryl (C₁₋₆ alkyl). In some embodiments, at least one of R^{9B} and R^{10B} can be an optionally substituted —O-aryl (C₁₋₆ alkyl). For example, both R^{9B} and R^{10B} can be an optionally substituted —O-benzyl. When substituted, the substituted —O-benzyl group can be substituted with 1, 2, 3 or more than 3 substituents. When more than two substituents are present, the substituents can be the same or different. In some embodiments, the —O-aryl group of the aryl (C₁₋₆ alkyl) can be a para-, ortho- or meta-substituted phenyl.

In some embodiments, at least one of R^{9B} and R^{10B} can be

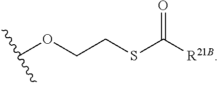

In some embodiments, R^{9B} and R^{10B} can be both

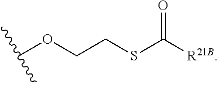

In some embodiments, at least one of R^{9B} and R^{10B} can be

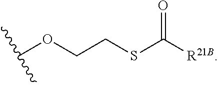

In some embodiments, R^{21B} can be hydrogen or deuterium. In other embodiments, R^{21B} can be an optionally substituted C₁₋₂₄ alkyl. In still other embodiments, R^{21B} can be an optionally substituted aryl (for example, an optionally substituted phenyl). In some embodiments, R^{21B} can be a C₁₋₆ alkyl, for example, methyl, ethyl, n-propyl, isopropyl, n-butyl, isobutyl, tert-butyl, pentyl (branched and straight-chained) and hexyl (branched and straight-chained). In some embodiments, R^{9B} and R^{10B} can be both an optionally substituted S-acylthioethoxy (SATE) group and form an optionally substituted SATE ester prodrug.

In some embodiments, R^{9B} and R^{10B} can be taken together to form an optionally substituted

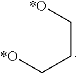

For example, when R^{9B} and R^{10B} can be taken together, the resulting moiety can be an optionally substituted

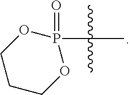 [SMILES: *P1(=O)OCCCO1]

When substituted, the ring can be substituted 1, 2, 3 or 3 or more times. When substituted with multiple substituents, the substituents can be the same or different. In some embodiments, the ring

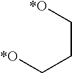

can be substituted with an optionally substituted aryl group and/or an optionally substituted heteroaryl. An example of a suitable heteroaryl is pyridinyl. In some embodiments, R^{9B} and R^{10B} can be taken together to form an optionally substituted

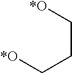

such as

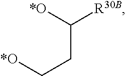

wherein R^{30B} can be an optionally substituted aryl, an optionally substituted heteroaryl or an optionally substituted heterocyclyl. In some embodiments, R^{9B} and R^{10B} can form an optionally substituted cyclic 1-aryl-1,3-propanyl ester (HepDirect) prodrug moiety. In this paragraph, the asterisks indicate the points of attachment of the moieties.

In some embodiments, R^{9B} and R^{10B} can be taken together to form an optionally substituted

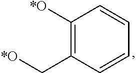

wherein the phosphorus and the moiety form a six-membered to ten-membered ring system. Example of an optionally substituted

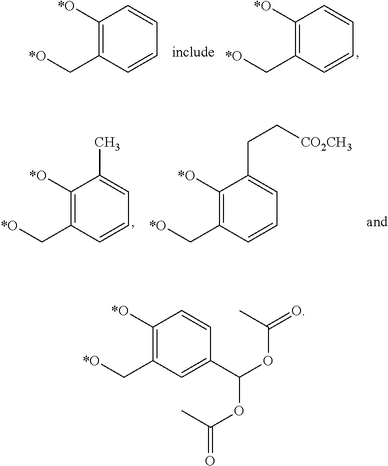

In some embodiments, R^{9B} and R^{10B} can form an optionally substituted cyclosaligenyl (cycloSal) prodrug. In this paragraph, the asterisks indicate the points of attachment of the moieties.

In other embodiments, R^{9B} can be an optionally substituted —O-aryl; and R^{10B} can be an optionally substituted N-linked amino acid or an optionally substituted N-linked amino acid ester derivative. In still other embodiments, R^{9B} can be an optionally substituted —O-heteroaryl; and R^{10B} can be an optionally substituted N-linked amino acid or an optionally substituted N-linked amino acid ester derivative.

In some embodiments, when R^{9B} can be an optionally substituted —O-aryl, R^{9B} can be an optionally substituted —O-phenyl. When the phenyl is substituted, the ring can be substituted 1, 2, 3 or more than 3 times. When substituted, the phenyl can be substituted at one or both ortho positions, one or both meta positions and/or the para position. In some embodiments, R^{9B} can be an unsubstituted —O-aryl. In some embodiments, R^{9B} can be an optionally substituted —O-naphthyl. In some embodiments, R^{9B} can be an unsubstituted —O-phenyl. In some embodiments, R^{9B} can be an unsubstituted —O-naphthyl.

In some embodiments, when R^{10B} can be an optionally substituted N-linked amino acid or an optionally substituted N-linked amino acid ester derivative, such as an optionally substituted N-linked α-amino acid or an optionally substituted N-linked α-amino acid ester derivative. Various amino acids are suitable, including those described herein. Examples of suitable amino acids include, but are not limited to, alanine, asparagine, aspartate, cysteine, glutamate, glutamine, glycine, proline, serine, tyrosine, arginine, histidine, isoleucine, leucine, lysine, methionine, phenylalanine, threonine, tryptophan and valine. In other embodiments, R^{10B} can be an optionally substituted N-linked amino acid ester derivative. Examples of suitable amino acid ester derivatives include, but are not limited to, an ester derivative of any of the following amino acids, alanine, asparagine, aspartate, cysteine, glutamate, glutamine, glycine, proline, serine, tyrosine, arginine, histidine, isoleucine, leucine, lysine, methionine, phenylalanine, threonine, tryptophan and valine. Additional examples of N-linked amino acid ester derivatives include, but are not limited to, an ester derivative of any of the following amino acids: alpha-ethyl-glycine, alpha.-propyl-glycine and beta-alanine. In some embodiments, the N-linked amino acid ester derivative can be selected from N-alanine isopropyl ester, N-alanine cyclohexyl ester, N-alanine neopentyl ester, N-valine isopropyl ester and N-leucine isopropyl ester.

In some embodiments, R^{10B} can be

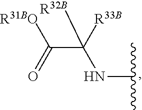

wherein R ^{31B} can be selected from hydrogen, deuterium, an optionally substituted C₁₋₆-alkyl, an optionally substituted C₃₋₆ cycloalkyl, an optionally substituted aryl, an optionally substituted aryl (C₁₋₆ alkyl) and an optionally substituted haloalkyl; R^{32B} can be selected from hydrogen, deuterium, an optionally substituted C₁₋₆ alkyl, an optionally substituted C₁₋₆ haloalkyl, an optionally substituted C₃₋₆ cycloalkyl, an optionally substituted C₆ aryl, an optionally substituted C₁₀ aryl and an optionally substituted aryl (C₁₋₆ alkyl); and R^{33B} can be hydrogen, deuterium or an optionally substituted C₁₋₄-alkyl; or R^{32B} and R^{33B} can be taken together to form an optionally substituted C₃₋₆ cycloalkyl.

In some embodiments, R^{32B} can be substituted by a variety of substituents. Suitable examples of substituents include, but are not limited to, N-amido, mercapto, alkylthio, an optionally substituted aryl, hydroxyl, an optionally substituted heteroaryl, carboxy and amino. In some embodiments R^{32B} can be hydrogen or deuterium. In some embodiments, R^{32B} can be an optionally substituted C₁₋₆-alkyl. In some embodiments, R^{33B} can be hydrogen or deuterium. In some embodiments R^{33B} can be an optionally substituted C₁₋₄ alkyl, such as methyl, ethyl, n-propyl, isopropyl, n-butyl, isobutyl or tert-butyl. In some embodiments R^{33B} can be methyl. In some embodiments, R^{31B} can be an optionally substituted C₁₋₆ alkyl. Examples of optionally substituted C₁₋₆-alkyls include optionally substituted variants of the following: methyl, ethyl, n-propyl, isopropyl, n-butyl, isobutyl, tea-butyl, pentyl (branched and straight-chained) and hexyl (branched and straight-chained). In some embodiments, R^{31B} can be methyl or isopropyl. In some embodiments, R^{31B} can he ethyl or neopentyl. In some embodiments, R^{31B} can be an optionally substituted C₃₋₆ cycloalkyl. Examples of optionally substituted C₃₋₆ cycloalkyls include optionally substituted variants of the following: cyclopropyl, cyclobutyl, cyclopentyl and cyclohexyl. Depending on the groups that are selected for R^{32B} and R^{33B}, the carbon to which R^{32B} and R^{33B} are attached may be a chiral center. In some embodiments, the carbon to which R^{32B} and R^{33B} are attached may be a (R)-chiral center. In other embodiments, the carbon to which R^{32B} and R^{33B} are attached may be a (S)-chiral center.

Examples of suitable

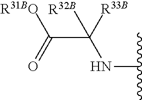

groups include the
following:

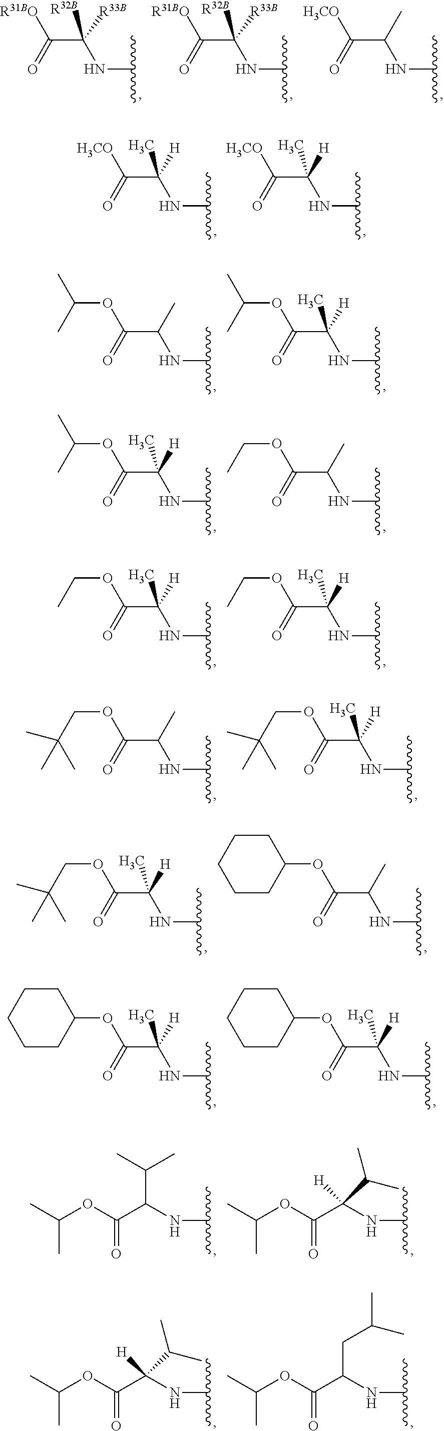

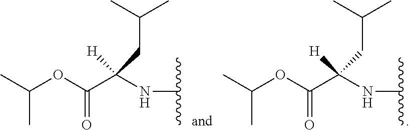 [SMILES: *N[C@H](CC(C)C)C(=O)OC(C)C ; *N[C@@H](CC(C)C)C(=O)OC(C)C]

In some embodiments, R^{9B} and R^{10B} can form an optionally substituted phosphoramidate prodrug, such as an optionally substituted aryl phosphoramidate prodrug. For example, R⁹ can be an —O-optionally substituted aryl and R^{10B} can be an optionally substituted N-linked amino acid or an optionally substituted N-linked amino acid ester derivative.

In some embodiments, both R^{9B} and R^{10B} can be independently an optionally substituted N-linked amino acid or an optionally substituted N-linked amino acid ester derivative, for example, both R^{9B} and R^{10B} can be an optionally substituted N-linked amino acid or an optionally substituted N-linked α-amino acid ester derivative. Various amino acids are suitable, including those described herein. Examples of suitable amino acids include, but are not limited to, alanine, asparagine, aspartate, cysteine, glutamate, glutamine, glycine, proline, serine, tyrosine, arginine, histidine, isoleucine, leucine, lysine, methionine, phenylalanine, threonine, tryptophan and valine. In other embodiments, both R^{9B} and R^{10B} can be independently an optionally substituted N-linked amino acid ester derivative. Examples of suitable amino acid ester derivatives include, but are not limited to, an ester derivative of any of the following amino acids, alanine, asparagine, aspartate, cysteine, glutamate, glutamine, glycine, proline, serine, tyrosine, arginine, histidine, isoleucine, leucine, lysine, methionine, phenylalanine, threonine, tryptophan and valine. Additional examples of N-linked amino acid ester derivatives include, but are not limited to, an ester derivative of any of the following amino acids: alpha-ethyl-glycine, alpha-propyl-glycine and beta-alanine. In some embodiments, the N-linked amino acid ester derivative can be selected from N-alanine isopropyl ester, N-alanine cyclohexyl ester, N-alanine neopentyl ester, N-valine isopropyl ester and N-leucine isopropyl ester. In some embodiments, R^{9B} and R^{10B} can form an optionally substituted phosphoric diamide prodrug.

In some embodiments, both R^{9B} and R^{10B} can be independently

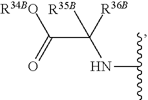

wherein R^{34B} can be selected from hydrogen, deuterium, an optionally substituted C₁₋₆-alkyl, an optionally substituted C₃₋₆ cycloalkyl, an optionally substituted aryl, an optionally substituted aryl (C₁₋₆ alkyl) and an optionally substituted haloalkyl; R^{35B} can be selected from hydrogen, deuterium, an optionally substituted C₁₋₆ alkyl, an optionally substituted C₁₋₆ haloalkyl, an optionally substituted C₃₋₆ cycloalkyl, an optionally substituted C₆ aryl, an optionally substituted C₁₀ aryl and an optionally substituted aryl (C₁₋₆ alkyl); and R^{36B} can be hydrogen, deuterium or an optionally substituted C₁₋₄-alkyl; or R^{35B} and R^{36B} can be taken together to form an optionally substituted C₃₋₆ cycloalkyl.

In some embodiments, R^{35B} can be substituted by a variety of substituents. Suitable examples of substituents include, but are not limited to, N-amido, mercapto, alkylthio, an optionally substituted aryl, hydroxyl, an optionally substituted heteroaryl, carboxy and amino. In some embodiments R^{35B} can be hydrogen or deuterium. In some embodiments. R^{35B} can be an optionally substituted C₁₋₆-alkyl. In some embodiments, R^{36B} can be hydrogen or deuterium. In some embodiments R^{36B} can be an optionally substituted C₁₋₄ alkyl, such as methyl, ethyl, n-propyl, isopropyl, n-butyl, isobutyl or tert-butyl. In some embodiments R^{36B} can be methyl. In some embodiments, R^{34B} can be an optionally substituted C₁₋₆ alkyl. Examples of optionally substituted C₁₋₆-alkyls include optionally substituted variants of the following: methyl, ethyl, n-propyl, isopropyl, n-butyl, isobutyl, ten-butyl, pentyl (branched and straight-chained) and hexyl (branched and straight-chained). In some embodiments, R^{34B} can be methyl or isopropyl. In some embodiments, R^{34B} can be ethyl or neopentyl. In some embodiments, R^{34B} can be an optionally substituted C₃₋₆ cycloalkyl. Examples of optionally substituted C₃₋₆ cycloalkyls include optionally substituted variants of the following: cyclopropyl, cyclobutyl, cyclopentyl and cyclohexyl. Depending on the groups that are selected for R^{35A} and R_{36A}, the carbon to which R^{35B} and R^{36B} are attached may be a chiral center. In some embodiments, the carbon to which R^{35B} and R^{36B} are attached may be a (R)-chiral center. In other embodiments, the carbon to which R^{35B} and R^{36B} are attached may be a (S)-chiral center.

Examples of suitable

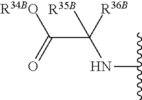

groups include the following:

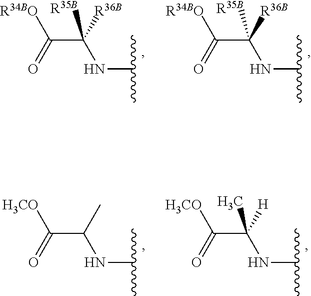

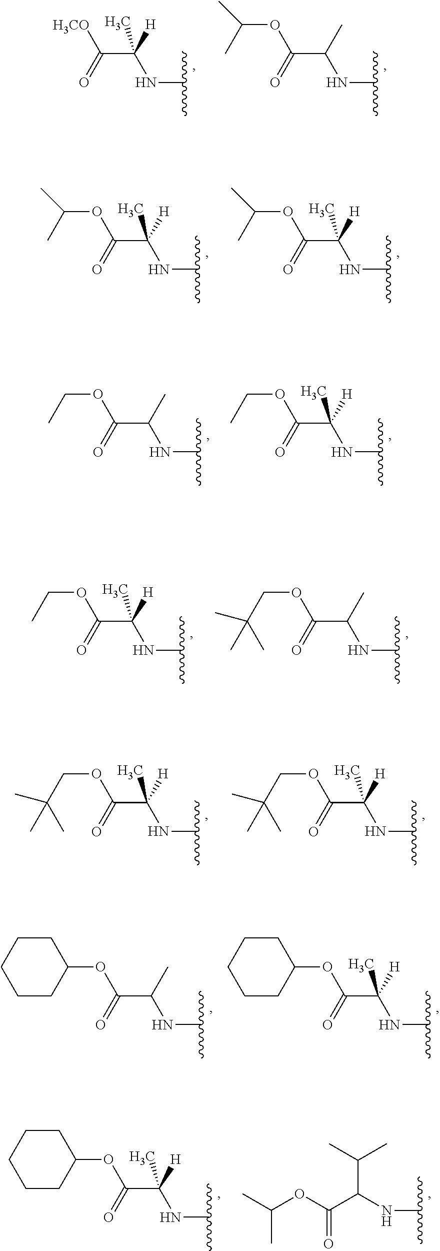

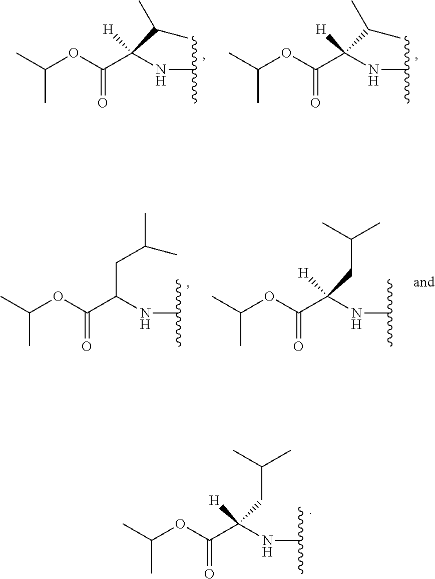

In some embodiments, R^{8B} and R^{10B} can be the same. In some embodiments. R^{9B} and R^{10B} can be different.

In some embodiments, R^{9B} and R^{10B} can be independently O⁻ or —OH. In other embodiments, R^{9B} can be

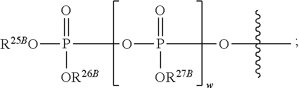

wherein w can be 0, R^{25B} and R^{26B} can be independently absent, hydrogen or deuterium; and R^{10B} can be O⁻ or —OH. Those skilled in the art understand that when R^{25B}, R^{26B} and R^{27B} are absent, the associated oxygen can have a negative charge. For example, when R^{26B} is absent, then the associated oxygen can have a negative charge, such that R^{9B} can be

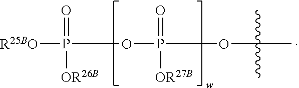

### When R^{9B} is

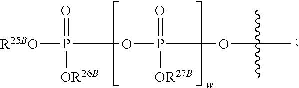

R^{25B} and R^{26B} are independently absent, hydrogen or deuterium, w is 0 and R^{10B} is O⁻ or —OH, a compound of Formula (II), or a pharmaceutically acceptable salt thereof, can be a diphosphate when Z^{1B} is O and an alpha-thiodiphosphate when Z^{1B} is S. In yet other embodiments R^{9B} can be

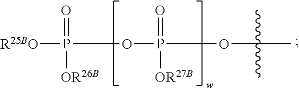

wherein w can be 1; R^{25B}, R^{26B} and R^{27B} can be independently absent, hydrogen or deuterium; and R^{10B} can be O⁻ or —OH. When R^{9B} is

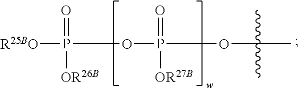

R^{25B}, R^{26B} and R^{27B} are independently absent, hydrogen or deuterium, w is 1 and is O⁻or −OH, a compound of Formula (II), or a pharmaceutically acceptable salt thereof, can be a triphosphate when Z^{1B} is O and an alpha-thiotriphosphate when Z^{1B} is S.

In some embodiment. R^{6B} can be —OH. In other embodiment, R^{6B} can be OC(═O)R″^{B}, wherein R″^{B} can be an optionally substituted C₁₋₂₄ alkyl. In some embodiments, R″^{B} can be a substituted C₁₋₁₂ alkyl. In other embodiments, R″^{B} can be an unsubstituted C₁₋₁₂ alkyl.

In some embodiment, R^{6B} can be an optionally substituted O-linked amino acid, such as an optionally substituted O-linked α-amino acid. Examples of suitable O-linked amino acids are described herein and include alanine, asparagine, aspartate, cysteine, glutamate, glutamine, glycine, proline, serine, tyrosine, arginine, histidine, isoleucine, leucine, lysine, methionine, phenylalanine, threonine, tryptophan, valine, ornithine, hypusine, 2-aminoisobutyric acid, dehydroalanine, gamma-aminobutyric acid, citrulline, beta-alanine, alpha-ethyl-glycine, alpha-propyl-glycine and norleucine. In some embodiments, the O-linked amino acid can have the structure

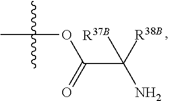

wherein R^{37B} can be selected from hydrogen, deuterium, an optionally substituted C₁₋₆ alkyl, an optionally substituted C₁₋₆ haloalkyl, an optionally substituted C₃₋₆ cycloalkyl, an optionally substituted C₆ aryl, an optionally substituted C₁₀ aryl and an optionally substituted aryl (C₁₋₆ alkyl); and R^{38B} can be hydrogen, deuterium or an optionally substituted C₁₋₄-alkyl; or R^{37B} and R^{38B} can be taken together to form an optionally substituted C₃₋₆ cycloalkyl.

When R^{37B} is substituted, R^{37B} can be substituted with one or more substituents selected from N-amido, mercapto, alkylthio, an optionally substituted aryl, hydroxy, an optionally substituted heteroaryl, O-carboxy and amino. In some embodiments, R^{37B} can be an unsubstituted C₁₋₆-alkyl, such as those described herein. In some embodiments. R^{37B} can be hydrogen or deuterium. In other embodiments, R^{37B} can be methyl. In some embodiments, R^{38B} can be hydrogen or deuterium. In other embodiments, R^{38B} can be an optionally substituted C₁₋₄-alkyl, such as methyl, ethyl, n-propyl, isopropyl, n-butyl, isobutyl and tert-butyl. In an embodiment, R^{38B} can be methyl. Depending on the groups that are selected for R^{37B} and R^{38B}, the carbon to which R^{37B} and R^{38B} are attached may be a chiral center. In some embodiment, the carbon to which R^{37B} and R^{38B} are attached may be a (R)-chiral center. In other embodiments, the carbon to which R^{37B} and R^{38B} are attached may be a (S)-chiral center.

Examples of suitable

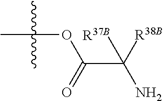

include the following:

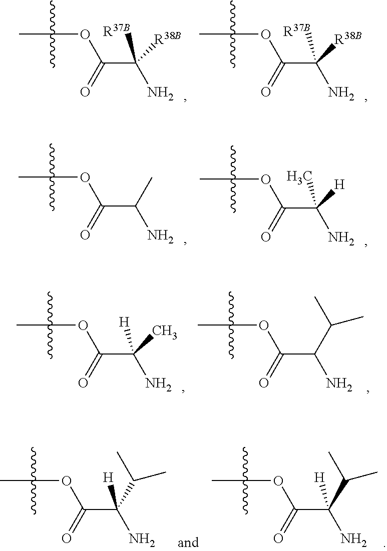

At the 3′-position, in some embodiments, R^{5B} can be hydrogen. In other embodiments, R^{5B} can be deuterium. For the I′-position, in some embodiments, R^{B} can be hydrogen. In other embodiments, R^{B} can be deuterium

In some embodiment, R^{7B} can be —OH. In other embodiment, R^{7B} can be fluoro. Instill other embodiment, R^{7B} can be chloro.

In some embodiment, R^{8B} can be an unsubstituted C₂₋₆ allenyl. For example, R^{8B} can be —C═C═CH₂. In other embodiments, R^{8B} can be an unsubstituted C₂₋₆ alkynyl. An example of an unsubstituted C₂₋₆ alkynyl is ethynyl.

In some embodiments, R^{2B} can be hydrogen. In other embodiments, R^{2B} can be deuterium. In some embodiments, R^{3B} can be hydrogen. In other embodiments, R^{3B} can be deuterium. In some embodiments, R^{2B} and R^{3B} can each be hydrogen. In other embodiments, R^{2B} and R^{3B} can each be deuterium. In still other embodiments, one of R^{2B} and R^{3B} can be hydrogen and the other of R^{2B} and R^{3B} can be deuterium.

In some embodiments, B^{1B} can be adenine or an adenine derivative. As used herein, an adenine derivative refers to adenine that is substituted and/or in which one or more of the nitrogens in the bicyclic ring(s) is replaced with a CR^{D}, wherein R^{D} can be hydrogen or deuterium or any of the other substituents from the “optionally substituted” list.

In some embodiments, B^{1B} can be

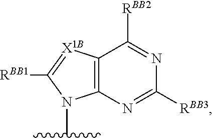

wherein X^{1B} can be N (nitrogen) or —CR^{BB6}; R^{BB1} can be hydrogen or deuterium; R^{BB2} can be NR^{BB4a}R^{BB4b}; R^{BB3} can be halogen or NR^{BB5a}R^{BB5b}, R^{BB4a} can be hydrogen or deuterium; R^{BB4b} can be selected from hydrogen, deuterium, an optionally substituted C₁₋₆ alkyl, an optionally substituted C₃₋₆ alkenyl, an optionally substituted C₃₋₆ cycloalkyl, —C(═O)R^{BB7} and —C(═O)OR^{BB8}; R^{BB5a} can be hydrogen or deuterium; R^{BB5b} can be selected from hydrogen, deuterium, an optionally substituted C₁₋₆ alkyl, an optionally substituted C₃₋₆ alkenyl, an optionally substituted C₃₋₆ cycloalkyl, —C(═O)R^{BB9} and —C(═O)OR^{BB10}; R^{BB6} can be selected from hydrogen, deuterium, halogen, —C≡N, an optionally substituted C₁₋₆ alkyl, an optionally substituted C₂₋₆ alkenyl, an optionally substituted C₂₋₆ alkynyl or —C(═O)NH₂; R^{BB7}, R^{BB8}, R^{BB9} and R^{BB10} can be independently selected from C₁₋₆ alkyl, C₂₋₆ alkenyl, C₂₋₆ alkynyl, C₃₋₆ cycloalkyl, C₅₋₁₀ cycloalkenyl, C₆₋₁₀ aryl, heteroaryl, heterocyclyl, aryl (C₁₋₆ alkyl), heteroaryl (C₁₋₆ alkyl) and heterocyclyl (C₁₋₆ alkyl).

In some embodiments, B^{1B} can be

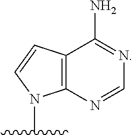 [SMILES: *n1ccc2c(N)ncnc21]

In still other embodiments, B^{1B} can be

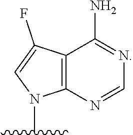 [SMILES: *n1cc(F)c2c(N)ncnc21]

In yet still other embodiments, B^{1B} can be

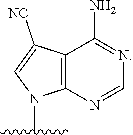 [SMILES: *n1cc(C#N)c2c(N)ncnc21]

In some embodiments, B^{1B} can be

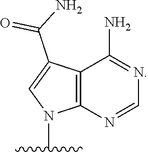 [SMILES: *n1cc(C(N)=O)c2c(N)ncnc21]

In other embodiments, B^{1B} can be

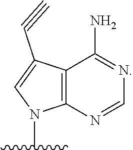 [SMILES: *n1cc(C#C)c2c(N)ncnc21]

In still other embodiments, B^{1B} can be

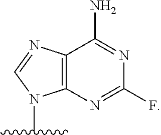 [SMILES: *n1cnc2c(N)nc(F)nc21]

In yet still other embodiments, B^{1B} can be

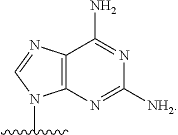 [SMILES: *n1cnc2c(N)nc(N)nc21]

In some embodiments of this paragraph, the shown amino group (—NH₂) can replaced with a N-carbamyl group having the structure of —(NH)—(C═O)—OR″^{D}, wherein R″^{D} can be an optionally substituted C₁₋₆ alkyl. In some embodiments, R″^{D} can be an unsubstituted C₁₋₆ alkyl. As examples wherein the shown amino group (—NH₂) is replaced with a N-carbamyl group, B^{1B} can be

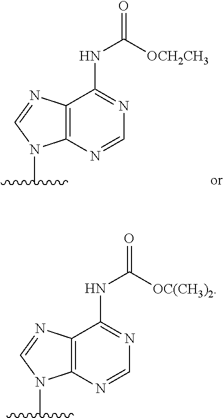

Examples of a compound of Formulae (I) and/or (II) include:

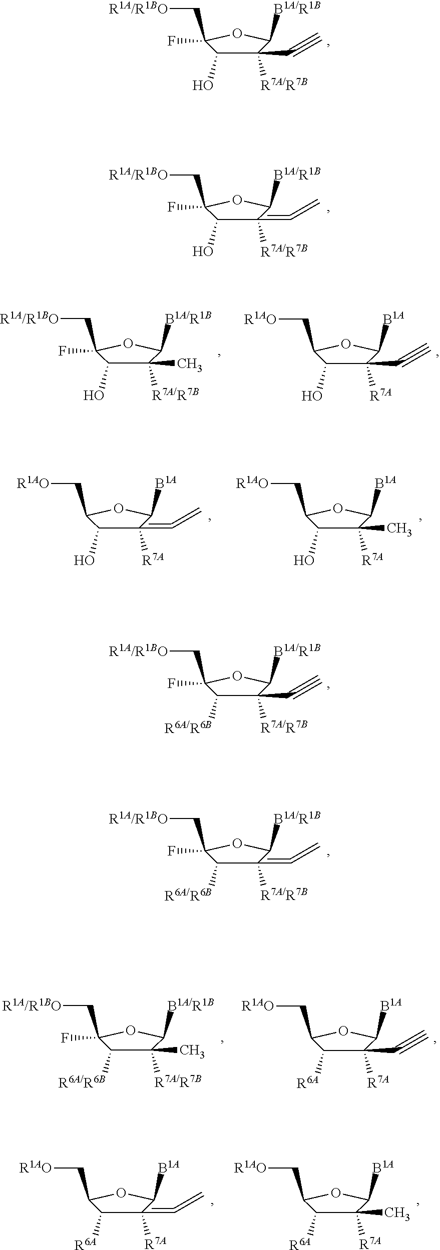

or a pharmaceutically acceptable salt of any of the foregoing. In some embodiments of this paragraph, R^{6A}/R^{6B} can be —OH. In some embodiments of this paragraph, R^{6A}/R^{6B} can be —OC(═O)R″^{A} or —OC(═O)R″^{B}, respectively, wherein each R″^{A} and each R″^{B} can be independently an optionally substituted C₁₋₂₄ alkyl. In some embodiments of this paragraph, R^{6A}/R^{6B} can be an optionally substituted O-linked amino acid, for example, an α-amino acid such as alanine or valine. In some embodiments of this paragraph, R^{7A}/R^{7B} can be —OH. In some embodiments of this paragraph, R^{7A} can be —OC(═O)R″^{B}, wherein R″^{B} can be an optionally substituted C₁₋₂₄ alkyl. In some embodiments of this paragraph, R^{7A}/R^{7B} can be fluoro. In some embodiments of this paragraph, R^{6A}/R^{6B} and R^{7A}/R^{7B} can each be —OH. In some embodiments of this paragraph, R^{6A} and R^{7A} can be —OC(═O)R″^{A} or —OC(═O)R″^{B}, respectively, wherein each R″^{A} and each R″^{B} can be independently an optionally substituted C₁₋₂₄ alkyl. In some embodiments of this paragraph, R^{6A}/R^{6B} can be —OH and R^{7A}/R^{7B} can be fluoro. In some embodiments of this paragraph, R^{6A} can be —OH and R^{7A} can be —OC(═O)R″^{B}, wherein R″^{B} can be an optionally substituted C₁₋₂₄ alkyl. In some embodiments of this paragraph, R^{6A}/R^{6B} can be —OC(═O)R″^{A} or —OC(═O)R″^{B}, respectively, wherein each R″^{A} and each R″^{B} can be independently an optionally substituted C₁₋₂₄ alkyl and R^{7A}/R^{7B} can be —OH. In some embodiments of this paragraph, R^{6A}/R^{6B} can be —OC(═O)R″^{A} or —OC(═O)R″^{B}, respectively, wherein each R″^{A} and each R″^{B} can be independently an optionally substituted C₁₋₂₄ alkyl and R^{7A}/R^{7B} can be fluoro. In some embodiments of this paragraph, R^{6A}/R^{6B} can be an optionally substituted O-linked amino acid (for example, an α-amino acid such as alanine or valine) and R^{7A}/R^{7B} can be —OH. In some embodiments of this paragraph, R^{6A}/R^{6B} can be an optionally substituted O-linked amino acid (for example, an α-amino acid such as alanine or valine) and R^{7A}/R^{7B} can be fluoro. In some embodiments of this paragraph, R^{6A} can be an optionally substituted O-linked amino acid (for example, an α-amino acid such as alanine or valine) and R^{7A} can be —OC(—O)R″^{B}, wherein R″^{B} can be an optionally substituted C₁₋₂₄ alkyl. In some embodiments, R^{1A}/R^{1B} can be hydrogen or deuterium. In some embodiments, R^{1A}/R^{1B} can be an optionally substituted acyl, for example, —C(═O)R″^{A1}, wherein R″^{A1} can be an optionally substituted C₁₋₁₂ alkyl or an unsubstituted C₁₋₈ alkyl. In some embodiments of this paragraph, R^{1A}/R^{1B} can be an optionally substituted O-linked amino acid, for example, an α-amino acid such as alanine or valine. In some embodiments of this paragraph, R^{1A}/R^{1B} can be a monophosphate. In some embodiments of this paragraph, R^{1A}/R^{1B} can be a diphosphate. In some embodiments of this paragraph, R^{1A}/R^{1B} can be a triphosphate. In some embodiments of this paragraph, R^{1A}/R^{1B} can be an optionally substituted bis(isopropyloxycarbonyloxymethyl) (bis(POC)) prodrug. In some embodiments of this paragraph, R^{1A}/R^{1B}can be an optionally substituted bis(pivaloyloxymethyl) (bis(POM)) prodrug. In some embodiments of this paragraph, R^{1A}/R^{1B} can be an optionally substituted SATE ester prodrug. In some embodiments of this paragraph, R^{1A}/R^{1B} can be an optionally substituted cyclic 1-aryl-1,3-propanyl ester (HepDirect) prodrug. In some embodiments of this paragraph R^{1A}/R^{1B} can be an optionally substituted cyclosaligenyl (cycloSal) prodrug. In some embodiments of this paragraph, R^{1A}/R^{1B} can be an optionally substituted phosphoramidate prodrug. In some embodiments of this paragraph, R^{1A}/R^{1B} can be an optionally substituted aryl phosphoramidate prodrug. In some embodiments of this paragraph, R^{1A}/R^{1B} can be an optionally substituted phosphonic diamide prodrug.

In some embodiments of this paragraph, B^{1A} can be

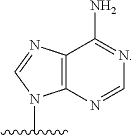 [SMILES: *n1cnc2c(N)ncnc21]

In some embodiments of this paragraph, B^{1A}/B^{1B} can be

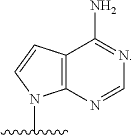 [SMILES: *n1ccc2c(N)ncnc21]

In some embodiments of this paragraph, B^{1A}/B^{1B} can be

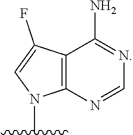 [SMILES: *n1cc(F)c2c(N)ncnc21]

In some embodiments of this paragraph, B^{1A}/B^{1B} can be

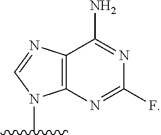 [SMILES: *n1cnc2c(N)nc(F)nc21]

In some embodiments of this paragraph, B^{1A}/B^{1B} can be

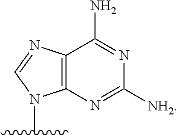 [SMILES: *n1cnc2c(N)nc(N)nc21]

In yet still other embodiments of this paragraph, B^{1A}/B^{1B} can be

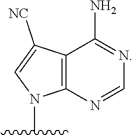 [SMILES: *n1cc(C#N)c2c(N)ncnc21]

In some embodiments of this paragraph, B^{1A}/B^{1B} can be

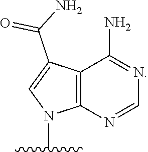 [SMILES: *n1cc(C(N)=O)c2c(N)ncnc21]

In other embodiments of this paragraph, B^{1A}/B^{1B} can be

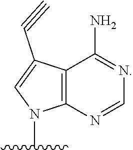 [SMILES: *n1cc(C#C)c2c(N)ncnc21]

In some embodiments of this paragraph, B^{1A}/B^{1B} can be

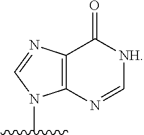 [SMILES: *n1cnc2c(=O)[nH]cnc21]

In some embodiments of this paragraph, B^{1A}/B^{1B} can be

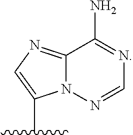 [SMILES: *c1cnc2c(N)ncnn12]

In some embodiments of this paragraph, B^{1A}/B^{1B} can be

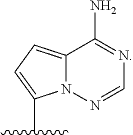 [SMILES: *c1ccc2c(N)ncnn12]

In some embodiments of this paragraph, B^{1A}/B^{1B} can be

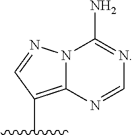 [SMILES: *c1cnn2c(N)ncnc12]

In some embodiments of this paragraph, B^{1A}/B^{1B} can be

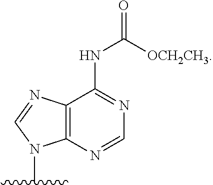 [SMILES: *n1cnc2c(NC(=O)OCC)ncnc21]

In some embodiments of this paragraph, B^{1A}/B^{1B} can be

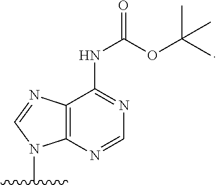 [SMILES: *n1cnc2c(NC(=O)OC(C)(C)C)ncnc21]

In some embodiments of this paragraph, B^{1A}/B^{1B} can be a base moiety provided in this paragraph wherein the shown amino group is replaced with a N-carbamyl group, such as those described herein (for example, —(NH)—(C═O)—OR″^{C} or —(NH)—(C═O)—OR″^{D}, wherein R″^{C} and R″^{D} can be independently an optionally substituted C₁₋₆ alkyl).

Examples of a compound of Formulae (I) and/or (II) include:

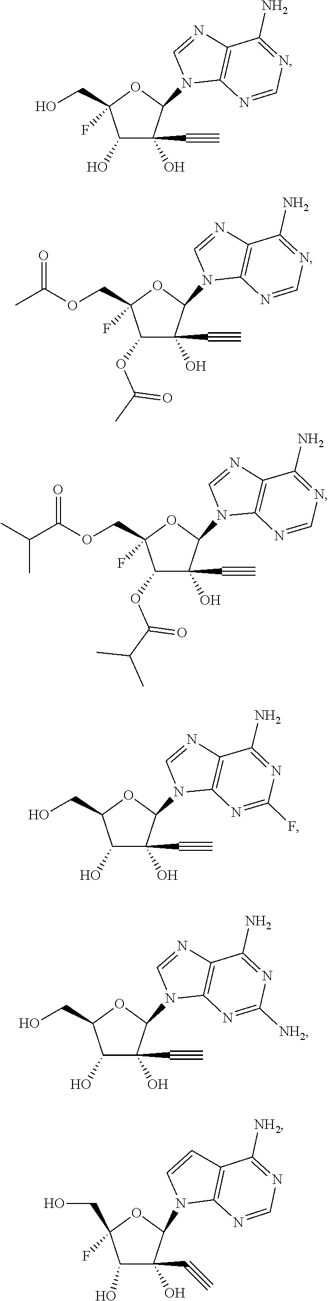 [SMILES: C#C[C@]1(O)[C@H](n2cnc3c(N)ncnc32)O[C@](F)(CO)[C@H]1O ; C#C[C@]1(O)[C@H](n2cnc3c(N)ncnc32)O[C@](F)(COC(C)=O)[C@H]1OC(C)=O ; C#C[C@]1(O)[C@H](n2cnc3c(N)ncnc32)O[C@](F)(COC(=O)C(C)C)[C@H]1OC(=O)C(C)C ; C#C[C@@]1(O)[C@H](O)[C@@H](CO)O[C@H]1n1cnc2c(N)nc(F)nc21 ; C#C[C@@]1(O)[C@H](O)[C@@H](CO)O[C@H]1n1cnc2c(N)nc(N)nc21 ; C#C[C@]1(O)[C@H](n2ccc3c(N)ncnc32)O[C@](F)(CO)[C@H]1O]

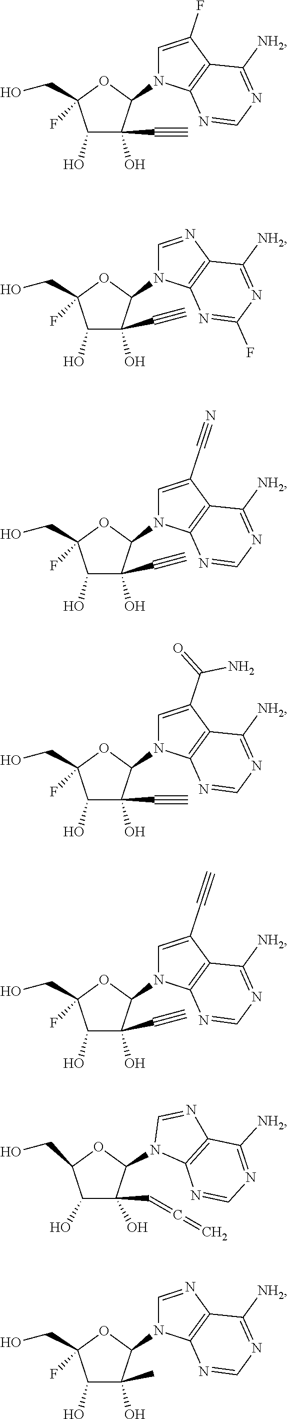

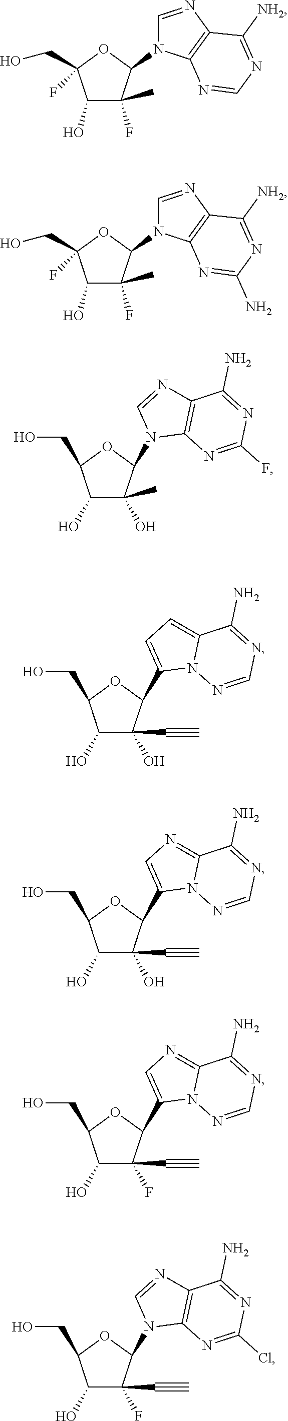

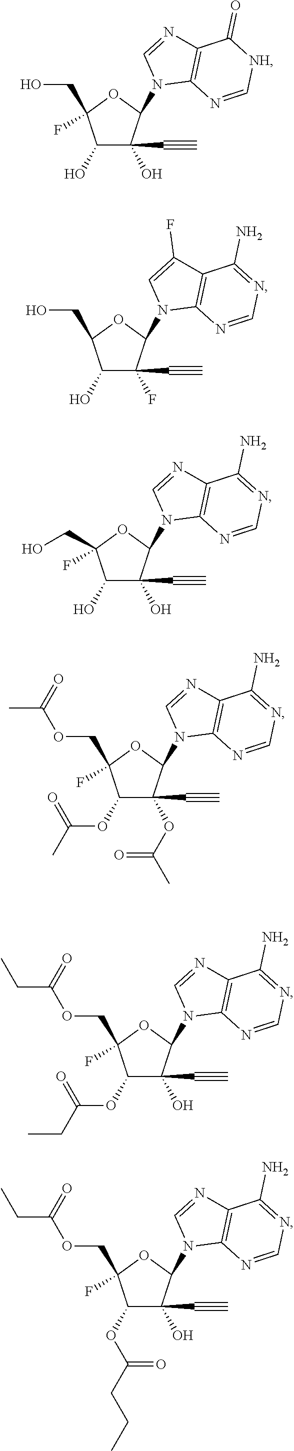 [SMILES: C#C[C@]1(O)[C@H](n2cnc3c(=O)[nH]cnc32)O[C@](F)(CO)[C@H]1O ; C#C[C@@]1(F)[C@H](O)[C@@H](CO)O[C@H]1n1cc(F)c2c(N)ncnc21 ; C#C[C@]1(O)[C@H](n2cnc3c(N)ncnc32)O[C@](F)(CO)[C@H]1O ; C#C[C@]1(OC(C)=O)[C@H](n2cnc3c(N)ncnc32)O[C@](F)(COC(C)=O)[C@H]1OC(C)=O ; C#C[C@]1(O)[C@H](n2cnc3c(N)ncnc32)O[C@](F)(COC(=O)CC)[C@H]1OC(=O)CC ; C#C[C@]1(O)[C@H](n2cnc3c(N)ncnc32)O[C@](F)(COC(=O)CC)[C@H]1OC(=O)CCC]

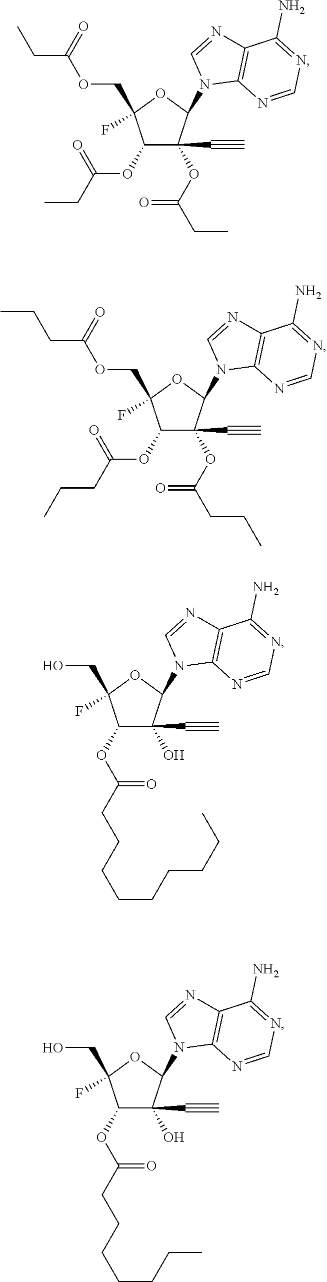

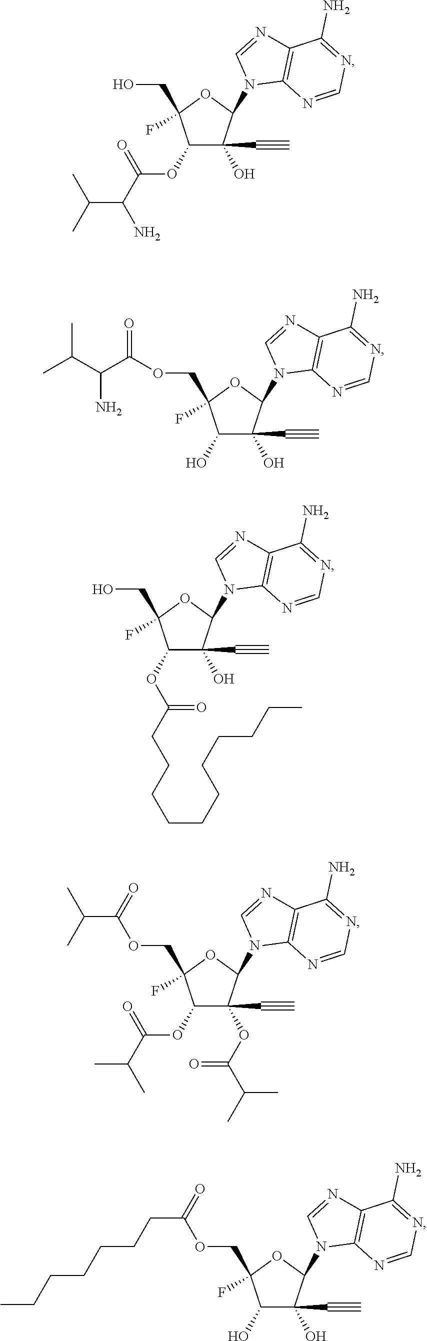 [SMILES: C#C[C@]1(O)[C@H](n2cnc3c(N)ncnc32)O[C@](F)(CO)[C@H]1OC(=O)C(N)C(C)C ; C#C[C@]1(O)[C@H](n2cnc3c(N)ncnc32)O[C@](F)(COC(=O)C(N)C(C)C)[C@H]1O ; C#C[C@]1(O)[C@H](n2cnc3c(N)ncnc32)O[C@](F)(CO)[C@H]1OC(=O)CCCCCCCCCCC ; C#C[C@]1(OC(=O)C(C)C)[C@H](n2cnc3c(N)ncnc32)O[C@](F)(COC(=O)C(C)C)[C@H]1OC(=O)C(C)C ; C#C[C@]1(O)[C@H](n2cnc3c(N)ncnc32)O[C@](F)(COC(=O)CCCCCCC)[C@H]1O]

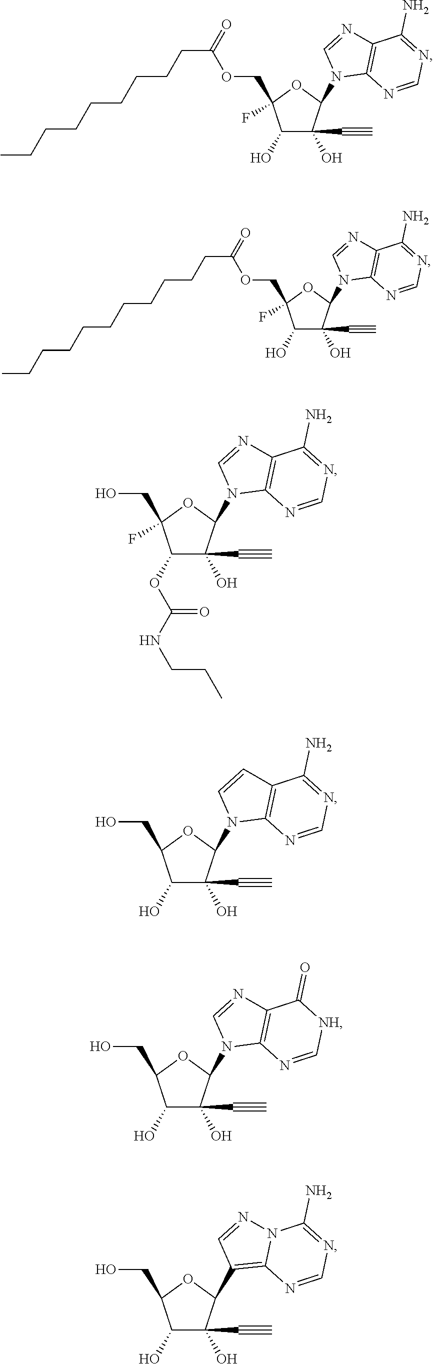 [SMILES: C#C[C@]1(O)[C@H](n2cnc3c(N)ncnc32)O[C@](F)(COC(=O)CCCCCCCCC)[C@H]1O ; C#C[C@]1(O)[C@H](n2cnc3c(N)ncnc32)O[C@](F)(COC(=O)CCCCCCCCCCC)[C@H]1O ; C#C[C@]1(O)[C@H](n2cnc3c(N)ncnc32)O[C@](F)(CO)[C@H]1OC(=O)NCCC ; C#C[C@@]1(O)[C@H](O)[C@@H](CO)O[C@H]1n1ccc2c(N)ncnc21 ; C#C[C@@]1(O)[C@H](O)[C@@H](CO)O[C@H]1n1cnc2c(=O)[nH]cnc21 ; C#C[C@@]1(O)[C@H](O)[C@@H](CO)O[C@H]1c1cnn2c(N)ncnc12]

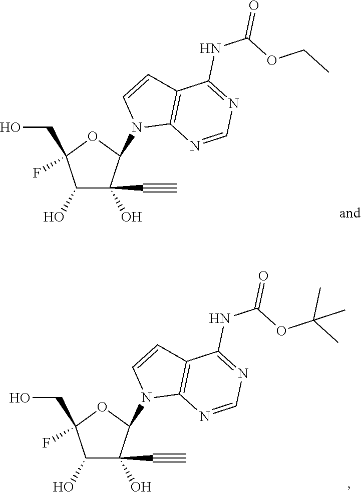 [SMILES: C#C[C@]1(O)[C@H](n2ccc3c(NC(=O)OCC)ncnc32)O[C@](F)(CO)[C@H]1O ; C#C[C@]1(O)[C@H](n2ccc3c(NC(=O)OC(C)(C)C)ncnc32)O[C@](F)(CO)[C@H]1O]

or a pharmaceuticallycceptable salt of any of the foregoing,

Additional examples of a compound of Formulae (I) and/or (II) include:

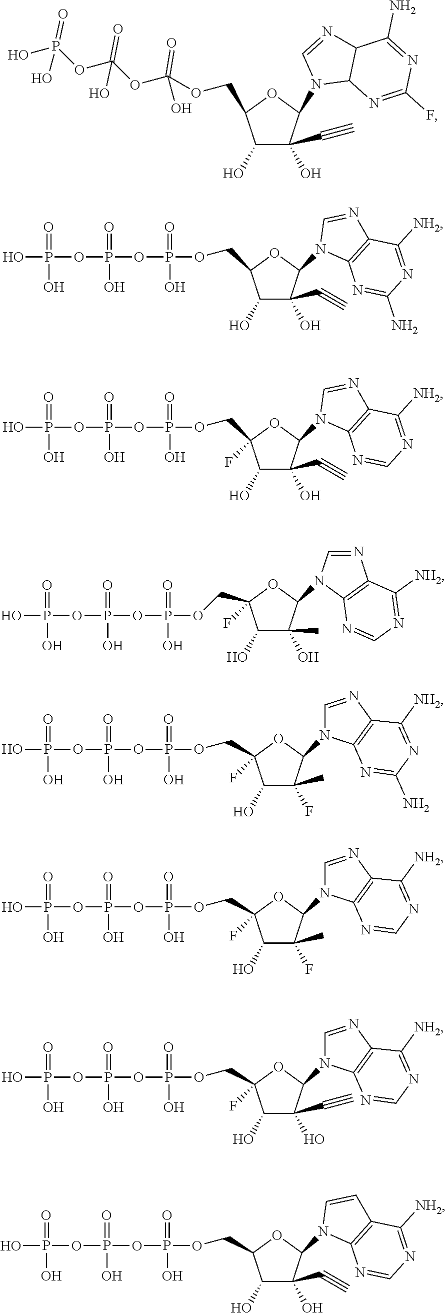

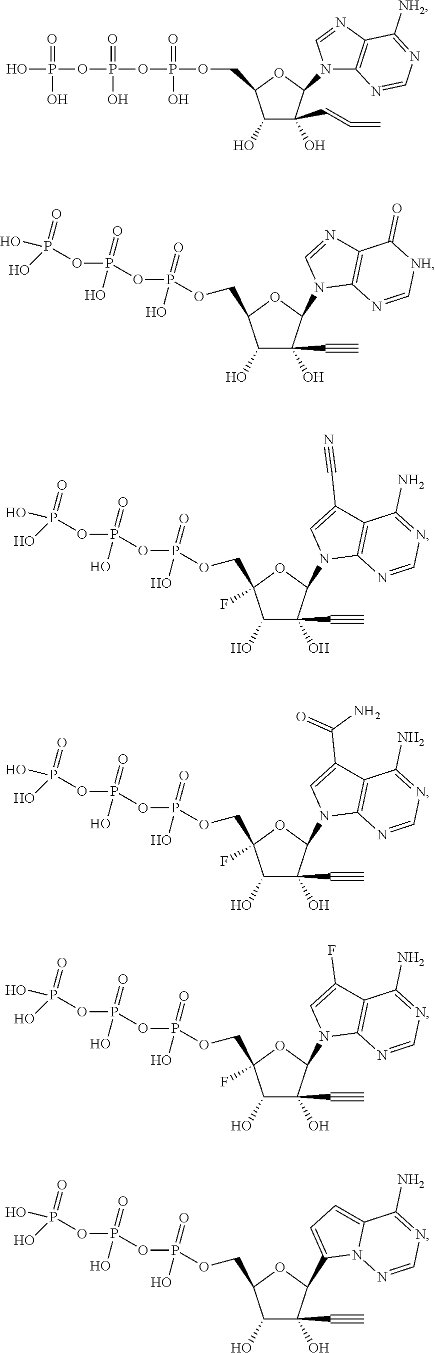

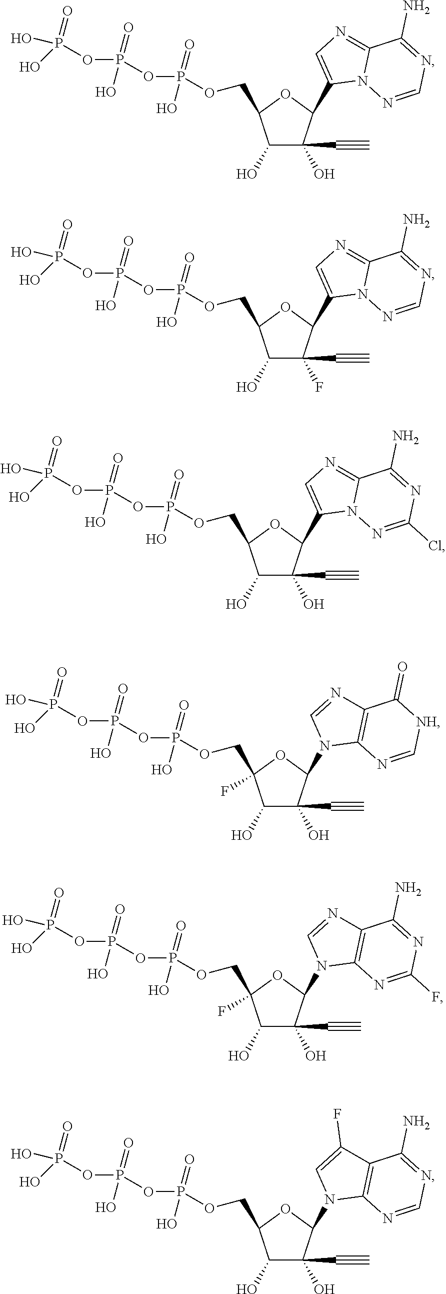

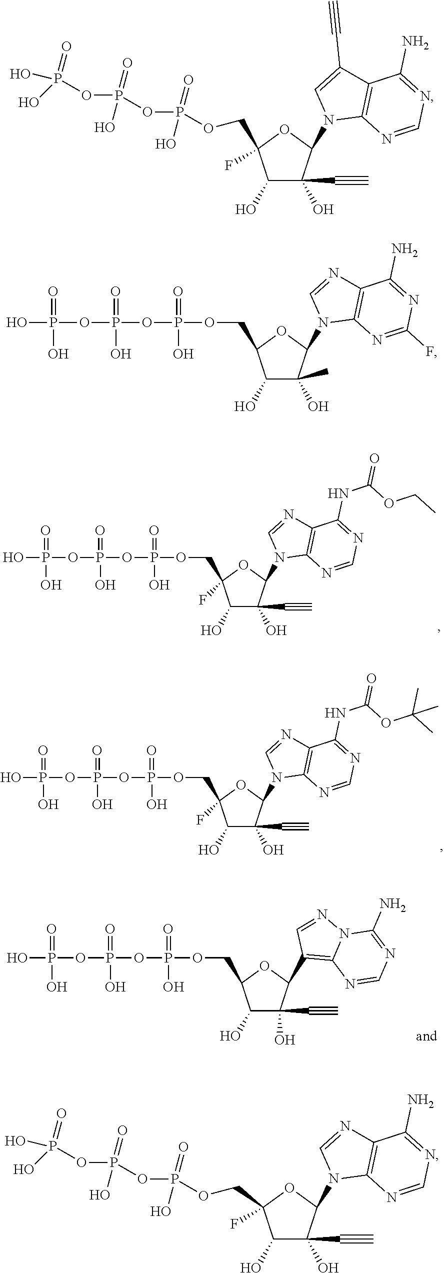

or pharmaceutically acceptable salt of any of any of the foregoing.

In some embodiments, B^{1A} cannot be

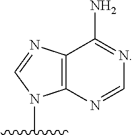 [SMILES: *n1cnc2c(N)ncnc21]

In some embodiments, B^{1A} cannot be

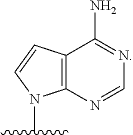 [SMILES: *n1ccc2c(N)ncnc21]

In some embodiments, B^{1B} cannot be

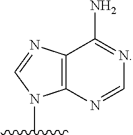 [SMILES: *n1cnc2c(N)ncnc21]

In some embodiments, B^{1B} cannot be

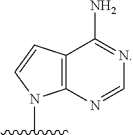 [SMILES: *n1ccc2c(N)ncnc21]

In some embodiments, R^{2A} and R^{3A} cannot each be —OH. In some embodiments, R^{2B} and R^{3B} cannot each be —OH. In some embodiments, R^{1A} cannot be hydrogen. In some embodiments, R^{1B} cannot be hydrogen.

In some embodiments of the compounds, methods and uses described herein, the compound of Formulae (I) and/or (II) cannot be

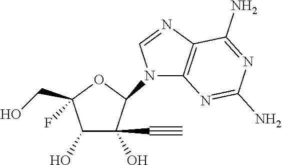 [SMILES: C#C[C@]1(O)[C@H](n2cnc3c(N)nc(N)nc32)O[C@](F)(CO)[C@H]1O]

or a pharmaceutically acceptable salt thereof. In some embodiments of the compounds, methods and uses described herein, the compound of Formulae (I) and/or (II) cannot be

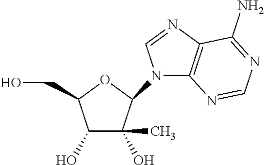 [SMILES: C[C@@]1(O)[C@H](O)[C@@H](CO)O[C@H]1n1cnc2c(N)ncnc21]

or a pharmaceutically acceptable salt thereof. In some embodiments of the compounds, methods and uses described herein, the compound of Formulae (I) and/or (II) cannot be

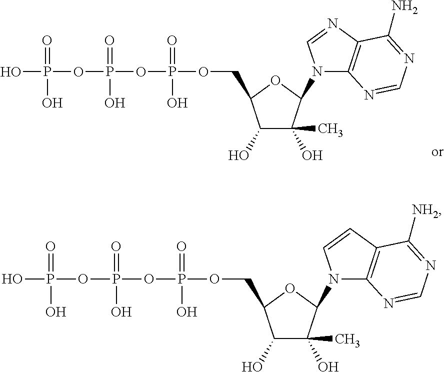 [SMILES: C[C@@]1(O)[C@H](O)[C@@H](COP(=O)(O)OP(=O)(O)OP(=O)(O)O)O[C@H]1n1cnc2c(N)ncnc21 ; C[C@@]1(O)[C@H](O)[C@@H](COP(=O)(O)OP(=O)(O)OP(=O)(O)O)O[C@H]1n1ccc2c(N)ncnc21]

or a pharmaceutically acceptable salt thereof.

In some embodiments of the compounds, methods and uses described herein, the compound of Formula (I) can be a compound or a pharmaceutically acceptable salt thereof as described herein, provided that when X¹ is N or CH, then (a) R^{4A} is fluoro, (b) R^{B3} is halogen or NR^{B5a}R^{B5b}, (c) R^{8A} is optionally substituted C₂₋₆ allenyl, or (d) any two or all three of said (a), (b) and (c) are present. In some embodiments of the compounds, methods and uses described herein, the compound of Formulae (I) and/or (II) can be a compound or a pharmaceutically acceptable salt thereof as described herein, provided that when X¹ is N or CH, R^{4A} is fluoro and R^{1A} is hydrogen or triphosphate, then R^{8A} is not methyl. In some embodiments of the compounds, methods and uses described herein, the compound of Formulae (I) and/or (II) can be a compound or a pharmaceutically acceptable salt thereof as described herein, provided that when X¹ is N or CH, R^{4A} is fluoro and R^{8A} is methyl, then R^{B3} is halogen or NR^{B5a}R^{B5b}. In some embodiments, when R^{4A} is hydrogen, then R^{8A} cannot be methyl. In some embodiments, when R^{4A} is deuterium, then R^{8A} cannot be methyl. In some embodiments, when R^{4A} is fluoro, then R^{8A} cannot be methyl. In some embodiments, when R^{4A} is hydrogen, then R^{8A} cannot be —CH═C═CH₂. In some embodiments, when R^{4A} is hydrogen, then R^{8A} cannot be a substituted or unsubstituted ethynyl. In some embodiments, when R^{4A} is hydrogen, then R^{8A} cannot be a substituted or unsubstituted C₃ or C₅ alkynyl. In some embodiments, when R^{4A} is hydrogen, then R^{1A} cannot be

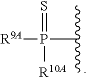

In some embodiments, when R^{8A} is methyl, then R^{1A} cannot be

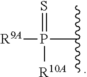

In some embodiments, when R^{8A} is methyl, then R^{1A} cannot be hydrogen. In some embodiments, when R^{8A} is an allenyl or an optionally substituted alkynyl, then R^{1A} cannot be

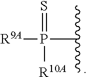

In some embodiments, a compound of Formulae (I) and/or (II), or a pharmaceutically acceptable salt of any of the foregoing, cannot be a compound, or a pharmaceutically acceptable salt thereof, described in U.S. 2013/0164261 or WO 2013/096680. In some embodiments, a compound of Formulae (I) and/or (II), or a pharmaceutically acceptable salt of any of the foregoing, cannot be a compound, or a pharmaceutically acceptable salt thereof, described in U.S. 2014/0179910, U.S. 2014/0179627 or WO 2014/100505. In some embodiments, a compound of Formulae (I) and/or (II), or a pharmaceutically acceptable salt of any of the foregoing, cannot be a compound, or a pharmaceutically acceptable salt thereof, described in U.S, 2012/0071434 or WO 2012/040127. In some embodiments, a compound of Formulae (I) and/or (II), or a pharmaceutically acceptable salt of any of the foregoing, cannot be a compound, or a pharmaceutically acceptable salt thereof, described in U.S. 2015/0105341 or WO 2015/054465.

By neutralizing the charge on the phosphorus moiety of Formulae (I) and/or (II), or a pharmaceutically acceptable salt of any of the foregoing, penetration of the cell membrane may be facilitated as a result of the increased lipophilicity of the compound. Once absorbed and taken inside the cell, the groups attached to the phosphorus can be easily removed by esterases, proteases and/or other enzymes. In some embodiments, the groups attached to the phosphorus can be removed by simple hydrolysis. Inside the cell, the phosphate thus released may then be metabolized by cellular enzymes to the diphosphate or the active triphosphate. Furthermore, in some embodiments, varying the substituents on a compound described herein, such as a compound of Formulae (I) and/or (II), or a pharmaceutically acceptable salt of any of the foregoing, can help maintain the efficacy of the compound by reducing undesirable effects.

In some embodiments, varying the substituents on a compound described herein, such as a compound of Formulae (I) and/or (II), or a pharmaceutically acceptable salt of any of the foregoing, can result in the phosphorous being a chiral center. In some embodiments, the phosphorous can be in the (R)-configuration. In some embodiments, the phosphorous can be in the (S)-configuration. Examples of the two configurations are:

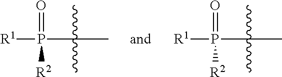

In some embodiments, a compound of Formulae (I) and/or (II), or a pharmaceutically acceptable salt of any of the foregoing, can be enriched in (R) or (S) configuration with respect to the phosphorous. For example, one of the (R) and (S) configuration with respect to the phosphorous atom can be present in an amount >50%, ≥75%, ≥90%, ≥95% or ≥99% compared to the amount of the other of the (R) or (S) configuration with respect to the phosphorous atom.

In some embodiments, a compound of Formulae (I) and/or (II), or a pharmaceutically acceptable salt of any of the foregoing, can inhibit the replication of a picornavirus because the compound of Formulae (I) and/or (II), or a pharmaceutically acceptable salt of any of the foregoing, can act as a chain terminator. For example, a compound of Formulae (I) and/or (II), or a pharmaceutically acceptable salt of any of the foregoing, can be incorporated into an RNA chain of a picornavirus, and then no further elongation is observed to occur.

In some embodiments, a compound of Formulae (I) and/or (II), or a pharmaceutically acceptable salt of any of the foregoing, can have increased metabolic and/or plasma stability. In some embodiments, a compound of Formulae (I) and/or (II), or a pharmaceutically acceptable salt of any of the foregoing, can be more resistant to hydrolysis and/or more resistant to enzymatic transformations. For example, a compound of Formulae (I) and/or (II), or a pharmaceutically acceptable salt of any of the foregoing, can have increased metabolic stability, increased plasma stability and can be more resistant to hydrolysis. In some embodiments, a compound of Formulae (I) and/or (II), or a pharmaceutically acceptable salt of any of the foregoing, can have improved properties. A non-limiting list of example properties include, but are not limited to, increased biological half-life, increased bioavailability (for example, increased oral bioavailability), increase potency, a sustained in vivo response, increased dosing intervals, decreased dosing amounts, decreased cytotoxicity, reduction in required amounts for treating disease conditions, reduction in viral load, reduction in plasma viral load, increase CD4+ T lymphocyte counts, reduction in time to seroconversion (i.e., the virus becomes undetectable in patient serum), increased sustained viral response, a reduction of morbidity or mortality in clinical outcomes, decrease in or prevention of opportunistic infections, increased subject compliance, increased compatibility with other medications and decreased side effects. In some embodiments, a compound of Formulae (I) and/or (II), or a pharmaceutically acceptable salt of any of the foregoing, can have a biological half-life of greater than 24 h. In some embodiments, a compound of Formulae (I) and/or (II), or a pharmaceutically acceptable salt of any of the foregoing, can have more potent antiviral activity (for example, a lower EC₅₀ in a picornavirus replicon assay) as compared to the current standard of care for a viral infection.

### Synthesis

Compounds of Formulae (I) and/or (II), or a pharmaceutically acceptable salt of any of the foregoing, and those described herein may be prepared in various ways, including those known to those skilled in the art. The routes shown and described herein are illustrative only and are not intended, nor are they to be construed, to limit the scope of the claims in any manner whatsoever. Those skilled in the art will be able to recognize modifications of the disclosed syntheses and to devise alternate routes based on the disclosures herein; all such modifications and alternate routes are within the scope of the claims. Examples of methods are described in the Examples below.

### Pharmaceutical Compositions

Some embodiments described herein relate to a pharmaceutical composition, that can include an effective amount of one or more compounds described herein (e.g., a compound of Formulae (I) and/or (II), or a pharmaceutically acceptable salt of any of the foregoing) and a pharmaceutically acceptable carrier, diluent, excipient or combination thereof. In some embodiments, the pharmaceutical composition can include a single diastereomer of a compound of Formulae (I) and/or (II), or a pharmaceutically acceptable salt of any of the foregoing, (for example, a single diastereomer is present in the pharmaceutical composition at a concentration of greater than 99% compared to the total concentration of the other diastereomers). In other embodiments, the pharmaceutical composition can include a mixture of diastereomers of a compound of Formulae (I) and/or (II), or a pharmaceutically acceptable salt of any of the foregoing. For example, the pharmaceutical composition can include a concentration of one diastereomer of >50%, ≥60%, ≥70%, ≥80%, ≥90%, ≥95%, or ≥98%, as compared to the total concentration of the other diastereomers. In some embodiments, the pharmaceutical composition includes a 1:1 mixture of two diastereomers of a compound of Formulae (I) and/or (II), or a pharmaceutically acceptable salt of any of the foregoing.

The term “pharmaceutical composition” refers to a mixture of one or more compounds disclosed herein with other chemical components, such as diluents or carriers. The pharmaceutical composition facilitates administration of the compound to an organism. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid and salicylic acid. Pharmaceutical compositions will generally be tailored to the specific intended route of administration. A pharmaceutical composition is suitable for human and/or veterinary applications.

The term “physiologically acceptable” defines a carrier, diluent or excipient that does not abrogate the biological activity and properties of the compound.

As used herein, a “carrier” refers to a compound that facilitates the incorporation of a compound into cells or tissues. For example, without limitation, dimethyl sulfoxide (DMSO) is a commonly utilized carrier that facilitates the uptake of many organic compounds into cells or tissues of a subject.

As used herein, a “diluent” refers to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

As used herein, an “excipient” refers to an inert substance that is added to a pharmaceutical composition to provide, without limitation, bulk, consistency, stability, binding ability, lubrication, disintegrating ability etc., to the composition. A “diluent” is a type of excipient

The pharmaceutical compositions described herein can be administered to a human patient per se, or in pharmaceutical compositions where they are mixed with other active ingredients, as in combination therapy, or carriers, diluents, excipients or combinations thereof. Proper formulation is dependent upon the route of administration chosen. Techniques for formulation and administration of the compounds described herein are known to those skilled in the art.

The pharmaceutical compositions disclosed herein may be manufactured in a manner that is itself known, e.g., by means of conventional mixing, dissolving, granulating, dragee-making, levigating, emulsifying, encapsulating, entrapping or tableting processes. Additionally, the active ingredients are contained in an amount effective to achieve its intended purpose. Many of the compounds used in the pharmaceutical combinations disclosed herein may be provided as salts with pharmaceutically compatible counterions.

Multiple techniques of administering a compound exist in the art including, but not limited to, oral, rectal, topical, aerosol, injection and parenteral delivery, including intramuscular, subcutaneous, intravenous, intramedullary injections, intrathecal, direct intraventricular, intraperitoneal, intranasal and intraocular injections.

One may also administer the compound in a local rather than systemic manner, for example, via injection of the compound directly into the infected area, often in a depot or sustained release formulation. Furthermore, one may administer the compound in a targeted drug delivery system, for example, in a liposome coated with a tissue-specific antibody. The liposomes will be targeted to and taken up selectively by the organ,

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions that can include a compound described herein formulated in a compatible pharmaceutical carrier may also be prepared, placed in an appropriate container and labeled for treatment of an indicated condition.

### Methods of Use

Some embodiments disclosed herein relate to a method of treating and/or ameliorating a Picornaviridae viral infection that can include administering to a subject infected with the Picornaviridae virus an effective amount of one or more compounds described herein (such as a compound of Formulae (I) and/or (II), or a pharmaceutically acceptable salt of any of the foregoing), or a pharmaceutical composition that includes a compound described herein (such as a compound of Formulae (I) and/or (II), or a pharmaceutically acceptable salt of any of the foregoing). Other embodiments disclosed herein relate to a method of treating and/or ameliorating a Picornaviridae viral infection that can include administering to a subject identified as suffering from the viral infection an effective amount of one or more compounds described herein (such as a compound of Formulae (I) and/or (II), or a pharmaceutically acceptable salt of any of the foregoing), or a pharmaceutical composition that includes a compound described herein (such as a compound of Formulae (I) and/or (II), or a pharmaceutically acceptable salt of any of the foregoing).

Some embodiments described herein relate to using one or more compounds described herein s a compound of Formulae (I) and/or (II), or a pharmaceutically acceptable salt of any of the foregoing), in the manufacture of a medicament for ameliorating and/or treating a Picornaviridae viral infection that can include administering to a subject infected with the Picornaviridae virus an effective amount of one or more compounds described herein (such as a compound of Formulae (I) and/or (II), or a pharmaceutically acceptable salt of any of the foregoing). Still other embodiments described herein relate to one or more compounds described herein (such as a compound of Formulae (I) and/or (II), or a pharmaceutically acceptable salt of any of the foregoing) that can be used for ameliorating and/or treating a Picornaviridae viral infection by administering to a subject infected with the Picornaviridae virus an effective amount of one or more compounds described herein, or a pharmaceutically acceptable salt thereof.

Some embodiments disclosed herein relate to methods of ameliorating and/or treating a Picornaviridae viral infection that can include contacting a cell infected with the Picornaviridae virus with an effective amount of one or more compounds described herein (such as a compound of Formulae (I) and/or (II), or a pharmaceutically acceptable salt of any of the foregoing), or a pharmaceutical composition that includes one or more compounds described herein (such as a compound of Formulae (I) and/or (II), or a pharmaceutically acceptable salt of any of the foregoing). Other embodiments described herein relate to using one or more compounds described herein (such as a compound of Formulae (I) and/or (II), or a pharmaceutically acceptable salt of any of the foregoing), in the manufacture of a medicament for ameliorating and/or treating a Picornaviridae viral infection that can include contacting a cell infected with the Picornaviridae virus with an effective amount of said compound(s), or a pharmaceutically acceptable salt thereof. Still other embodiments described herein relate to one or more compounds described herein (such as a compound of Formulae (I) and/or (II), or a pharmaceutically acceptable salt of any of the foregoing), that can be used for ameliorating and/or treating a Picornaviridae viral infection by contacting a cell infected with the Picornaviridae virus with an effective amount of said compound(s), or a pharmaceutically acceptable salt thereof.

Some embodiments disclosed herein relate to methods of inhibiting replication of a Picornaviridae virus that can include contacting a cell infected with the Picornaviridae virus with an effective amount of one or more compounds described herein (such as a compound of Formulae (I) and/or (II), or a pharmaceutically acceptable salt of any of the foregoing), or a pharmaceutical composition that includes one or more compounds described herein (such as a compound of Formulae (I) and/or (II), or a pharmaceutically acceptable salt of any of the foregoing). Other embodiments described herein relate to using one or more compounds described herein (such as a compound of Formulae (I) and/or (II), or a pharmaceutically acceptable salt of any of the foregoing), in the manufacture of a medicament for inhibiting replication of a Picornaviridae virus that can include contacting a cell infected with the Picornaviridae virus with an effective amount of said compound(s), or a pharmaceutically acceptable salt thereof. Still other embodiments described herein relate to a compound described herein (such as a compound of Formulae (I) and/or (II), or a pharmaceutically acceptable salt of any of the foregoing), that can be used for inhibiting replication of a Picornaviridae virus by contacting a cell infected with the Picornaviridae virus with an effective amount of said compound(s), or a pharmaceutically acceptable salt thereof. In some embodiments, a compound of Formulae (I) and/or (II), or a pharmaceutically acceptable salt of any of the foregoing, can inhibit a RNA dependent RNA polymerase of a Picornaviridae virus, and thus, inhibit the replication of RNA. In some embodiments, a polymerase of a Picornaviridae virus can be inhibited by contacting a cell infected with the Picornaviridae virus with a compound described herein (such as a compound of Formulae (I) and/or (II), or a pharmaceutically acceptable salt of any of the foregoing).

In some embodiments, the Picornaviridae virus can be selected from an Aphthovirus, an Enterovirus, a Rhinovirus, a Hepatovirus and a Parechovirus. Within the Enterovirus genus, there are several species of Enteroviruses including enterovirus A, enterovirus B, enterovirus C, enterovirus D, enterovirus E, enterovirus F, enterovirus G, enterovirus H, enterovirus J. Each Enterovirus species includes several serotypes. Examples of Enterovirus serotypes include the following: poliovirus 1, poliovirus 2, poliovirus 3, echovirus 1, echovirus 2, echovirus 3, echovirus 4, echovirus 5, echovirus 6, echovirus 7, echovirus 9, echovirus 11, echovirus 12, echovirus 13, echovirus 14, echovirus 15, echovirus 16, echovirus 17, echovirus 18, echovirus 19, echovirus 20, echovirus 21, echovirus 24, echovirus 25, echovirus 26, echovirus 27, echovirus 29, echovirus 30, echovirus 31, echovirus 32, echovirus 33, enterovirus 68, enterovirus 69, enterovirus 70, enterovirus 71 and viluisk human encephalomyelitis virus. In some embodiments, a compound described herein (for example, a compound of Formulae (I) and/or (II), or a pharmaceutical acceptable salt of any of the foregoing) can ameliorate and/or treat an Enterovirus infection. For example, by administering an effective amount of a compound of Formulae (I) and/or (II), or a pharmaceutical acceptable salt of any of the foregoing, to a subject infected with the Enterovirus and/or by contacting a cell infected with the Enterovirus with an effective amount of a compound of Formulae (I) and/or (II), or a pharmaceutical acceptable salt of any of the foregoing. In some embodiments, a compound described herein (for example, a compound of Formulae (I) and/or (II), or a pharmaceutical acceptable salt of any of the foregoing) can inhibit replication of an Enterovirus. In some embodiments, a compound of Formulae (I) and/or (II), or a pharmaceutical acceptable salt of any of the foregoing, can he effective against an Enterovirus, and thereby ameliorate one or more symptoms of an Enterovirus infection. In some embodiments, the Enterovirus can be Enterovirus A. In other embodiments, the Enterovirus can be Enterovirus B. In still other embodiments, the Enterovirus can be Enterovirus C. In yet still other embodiments, the Enterovirus can be Enterovirus D. In other embodiments, the Enterovirus can be Enterovirus E. In still other embodiments, the Enterovirus can be Enterovirus F. In yet still other embodiments, the Enterovirus can be Enterovirus G. In some embodiments, the Enterovirus can be Enterovirus H. In other embodiments, the Enterovirus can be Enterovirus J.

Coxsackieviruses are divided into group A and group B. Group A coxsackieviruses were noted to cause flaccid paralysis, while group B coxsackieviruses were noted to cause spastic paralysis. Over 20 serotypes of group A (CV-A1, CV-A2, CV-A3, CV-A4, CV-A5, CV-A6, CV-A7, CV-A8, CV-A9, CV-A10, CV-A11, CV-A12, CV-A13, CV-A14, CV-A15, CV-A16, CV-A17, CV-A18, CV-A19, CV-A20, CV-A21, CV-A22 and CV-A23) and 6 serotypes of group B (CV-B1, CN-B2, CV-B CV-B4, CV-B5 and CV-B6) are recognized. No specific treatment for coxsackievirus infections is currently approved. In some embodiments, a compound described herein (for example, a compound of Formulae (I) and/or (II), or a pharmaceutical acceptable salt of any of the foregoing) can ameliorate and/or treat a coxsackievirus infection. In some embodiments, a compound described herein (for example, a compound of Formulae (I) and/or (II), or a pharmaceutical acceptable salt of any of the foregoing) can inhibit replication of a coxsackievirus. In some embodiments, a compound of Formulae (I) and/or (II), or a pharmaceutical acceptable salt of any of the foregoing, can be effective against a coxsackievirus as demonstrated by the amelioration of one or more symptoms of a coxsackievirus infection. In some embodiments, a coxsackievirus infection can be ameliorated, treated and/or inhibited by administering an effective amount of a compound of Formulae (I) and/or (II), or a pharmaceutical acceptable salt of any of the foregoing, to a subject infected with the coxsackievirus and/or by contacting a cell infected with the coxsackievirus with an effective amount of a compound of Formulae (I) and/or (II), or a pharmaceutical acceptable salt of any of the foregoing. In some embodiments, the coxsackievirus can be a coxsackievirus A. In other embodiments, the coxsackievirus can be a coxsackievirus B. In some embodiments, a compound described herein (one or more a compound of Formulae (I) and/or (II), or a pharmaceutical acceptable salt of any of the foregoing) can ameliorate and/or treat hand, food and mouth disease caused by a coxsackie A virus.

Additional species within the Enterovirus genus includes rhinovirus A, rhinovirus B and rhinovirus C. In some embodiments, a compound described herein (for example, a compound of Formulae (I) and/or (II), or a pharmaceutical acceptable salt of any of the foregoing) can ameliorate and/or treat a Rhinovirus infection. In some embodiments, a compound described herein (for example, a compound of Formulae (I) and/or (II), or a pharmaceutical acceptable salt of any of the foregoing) can inhibit replication of a Rhinovirus. In some embodiments, a compound described herein (for example, a compound of Formulae (I) and/or (II), or a pharmaceutical acceptable salt of any of the foregoing) can be effective against multiple serotypes of a Rhinovirus. For example, a compound of Formulae (I) and/or (II), or a pharmaceutical acceptable salt of any of the foregoing, can be used to ameliorate and/or treat an infection caused by 2, 5, 10, 20, 40, 60, 80 or more serotypes of a Rhinovirus. In some embodiments, a compound of Formulae (I) and/or (II), or a pharmaceutical acceptable salt of any of the foregoing, can be effective against Rhinovirus, and thereby ameliorating one or more symptoms of a Rhinovirus infection. In some embodiments, a Rhinovirus infection can be ameliorated, treated and/or inhibited by administering an effective amount of a compound of Formulae (I) and/or (II), or a pharmaceutical acceptable salt of any of the foregoing, to a subject infected with the Rhinovirus and/or by contacting a cell infected with the Rhinovirus. In some embodiments, the Rhinovirus can be rhinovirus A. In other embodiments, the Rhinovirus can be rhinovirus B. In still other embodiments, the Rhinovirus can be rhinovirus C.

Another species of Enterovirus is Hepatovirus. Hepatitis A is a serotype of Hepatovirus. Several human genotypes of Hepatitis A are known, IA, IB, IIA, IIB, IIIA and IIIB. Genotype I is the most common. To date, there is no specific therapy for treating a hepatitis A infection. Rather, treatment is supportive in nature. In some embodiments, a compound described herein (for example, a compound of Formulae (I) and/or (II), or a pharmaceutical acceptable salt of any of the foregoing) can ameliorate and/or treat a Hepatovirus infection, such as a hepatitis A virus infection. In some embodiments, a compound described herein (for example, a compound of Formulae (I) and/or (II), or a pharmaceutical acceptable salt of any of the foregoing can inhibit replication of a Hepatovirus (for example, a hepatitis A virus). In some embodiment, a compound of Formulae (I) and/or (II), or a pharmaceutical acceptable salt of any of the foregoing, can treat and/or ameliorate an infection caused by a genotype I of hepatitis A. In some embodiments, a compound of Formulae (I) and/or (II), or a pharmaceutical acceptable salt of any of the foregoing, is effective against more than one genotype of hepatitis A, for example, 2, 3, 4, 5 or 6 genotypes of hepatitis A. In some embodiments, a Hepatovirus infection can be ameliorated, treated and/or inhibited by administering an effective amount of a compound of Formulae (I) and/or (II), or a pharmaceutical acceptable salt of any of the foregoing, to a subject infected with the Hepatovirus and/or by contacting a cell infected with the Hepatovirus with an effective amount of a compound of Formulae (I) and/or (II), or a pharmaceutical acceptable salt of any of the foregoing.

Parechovirus is another species of Enterovirus. Serotypes of parechovirus includes human parechovirus 1 (echovirus 22), human parechovirus 2 (echovirus 23), human parechovirus 3, human parechovirus 4, human parechovirus 5 and human parechovirus 6. In some embodiments, a compound described herein (for example, a compound of Formulae (I) and/or (II), or a pharmaceutical acceptable salt of any of the foregoing) can ameliorate and/or treat a parechovirus infection. In some embodiments, a compound described herein (for example, a compound of Formulae (I) and/or (II), or a pharmaceutical acceptable salt of any of the foregoing) can inhibit replication of a parechovirus. In some embodiments, a compound of Formulae (I) and/or (II), or a pharmaceutical acceptable salt of any of the foregoing, is effective against more than one serotype of a parechovirus. In some embodiments, a parechovirus infection can be ameliorated, treated and/or inhibited by administering an effective amount of a compound of Formulae (I) and/or (II), or a pharmaceutical acceptable salt of any of the foregoing, to a subject infected with the parechovirus and/or by contacting a cell infected with the parechovirus with an effective amount of a compound of Formulae (I) and/or (II), or a pharmaceutical acceptable salt of any of the foregoing.

Other genera of Picornaviridae virus include the following: Aquamavirus, Avihepatovirus, Cardiovirus, Cosavirus, Dicipivirus, Erbovirus, Kobuvirus, Megrivirus, Salivirus, Sapelovirus, Senecavirus, Teschovirus and Tremovirus. In some embodiments, a compound described herein (for example, a compound of Formulae (I) and/or (II), or a pharmaceutical acceptable salt of any of the foregoing) can ameliorate and/or treat a picornavirus infection caused by a virus selected from Aquamavirus, Avihepatovirus, Cardiovirus, Cosavirus, Dicipivirus, Erbovirus, Kobuvirus, Megrivirus, Salivirus, Sapelovirus, Senecavirus, Teschovirus and Tremovirus. In some embodiments, a compound described herein (for example, a compound of Formulae (I) and/or (II), or a pharmaceutical acceptable salt of any of the foregoing) can inhibit replication of a Picornaviridae virus selected from Aquainavirus, Avihepatovirus, Cardiovirus, Cosavirus, Dicipivirus, Erbovirus, Kobuvirus, Megrivirus, Salivirus, Sapelovirus, Senecavirus, Teschovirus and Tremovirus. A compound described herein (for example, a compound of Formulae (I) and/or (II), or a pharmaceutical acceptable salt of any of the foregoing) can ameliorate, treat and/or inhibit an infection caused by a virus selected from Aquamavirus, Avihepatovirus, Cardiovirus, Cosavirus, Dicipivirus, Erbovirus, Kobuvirus, Megrivirus, Salivirus, Sapelovirus, Senecavirus, Teschovirus and Tremovirus by administering an effective amount of a compound described herein to a subject infected by the virus and/or by contacting a cell infected with the virus with an effective amount of a compound described herein, or a pharmaceutically acceptable salt thereof.

In some embodiments, an effective amount of a compound of Formulae (I) and/(II), or a pharmaceutical acceptable salt of any of the foregoing, or a pharmaceutical composition that includes an effective amount of one or more compounds of Formulae (I) and/or (II), or a pharmaceutical acceptable salt of any of the foregoing, can be effective to treat an infection caused by more than one genera of Picornaviridae virus. In some embodiments, a compound described herein (for example, a compound of Formulae (I) and/or (II), or a pharmaceutical acceptable salt of any of the foregoing) can be used to ameliorate and/or treat an infection caused by more than one species of a Picornaviridae virus. As an example, a compound of Formulae (I) and/or (II), or a pharmaceutical acceptable salt of any of the foregoing, can be used to ameliorate and/or treat an infection caused by 2, 3, 4, 5, or more species of an Enterovirus. In some embodiments, a compound described herein (for example, a compound of Formulae (I) and/or (II), or a pharmaceutical acceptable salt of any of the foregoing) can be effective to treat an infection caused by multiple serotypes of a Picornaviridae virus described herein. For example, a compound described herein (one or more a compound of Formulae (I) and/or (II), or a pharmaceutical acceptable salt of any of the foregoing) can be effective to treat an infection caused by 2, 5, 10, 15 or more serotypes of Picornaviridae.

Various indicators for determining the effectiveness of a method for treating an Picornaviridae viral infection are known to those skilled in the art. Example of suitable indicators include, but are not limited to, a reduction in viral load, a reduction in viral replication, a reduction in time to seroconversion (virus undetectable in patient serum), a reduction of morbidity or mortality in clinical outcomes, a reduction in side effects of treatment and/or other indicator(s) of disease response. Further indicators include one or more overall quality of life health indicators, such as reduced illness duration, reduced illness severity,reduced time to return to normal health and normal activity, and reduced time to alleviation of one or more symptoms. In some embodiments, a compound of Formulae (I) and/or (II), or a pharmaceutically acceptable salt of any of the foregoing, can result in the reduction, alleviation or positive indication of one or more of the aforementioned indicators compared to an untreated subject. Effects/symptoms of a Picornaviridae viral infection are described herein, and include, but are not limited to, fever, blisters, rash, meningitis, conjunctivitis, acute hemorrhagic conjunctivitis (AHC), sore throat, nasal congestion, runny nose, sneezing, coughing, loss of appetite, muscle aches, headache, fatigue, nausea, jaundice, encephalitis, herpangina, myocarditis, pericarditis, meningitis, Bornholm disease, myalgia, nasal congestion, muscle weakness, loss of appetite, fever, vomiting, abdominal pain, abdominal discomfort, dark urine and muscle pain.

Some embodiments disclosed herein relate to a method of treating and/or ameliorating a Flaviviridae viral infection that can include administering to a subject infected with the Flaviviridae virus an effective amount of one or more compounds described herein (such as a compound of Formulae (I) and/or (II), or a pharmaceutically acceptable salt of any of the foregoing), or a pharmaceutical composition that includes a compound described herein (such as a compound of Formulae (I) and/or (II), or a pharmaceutically acceptable salt of any of the foregoing). Other embodiments disclosed herein relate to a method of treating and/or ameliorating a Flaviviridae viral infection that can include administering to a subject an effective amount of one or more compounds described herein (such as a compound of Formulae (I) and/or (II), or a pharmaceutically acceptable salt of any of the foregoing), or a pharmaceutical composition that includes a compound described herein (such as a compound of Formulae (I) and/or (II), or a pharmaceutically acceptable salt of any of the foregoing). Some embodiments described herein relate to using one or more compounds described herein (such as a compound of Formulae (I) and/or (II), or a pharmaceutically acceptable salt of any of the foregoing), in the manufacture of a medicament for ameliorating and/or treating a Flaviviridae viral infection that can include administering an effective amount of one or more compounds described herein (such as a compound of Formulae (I) and/or (II), or a pharmaceutically acceptable salt of any of the foregoing). Still other embodiments described herein relate to one or more compounds described herein (such as a compound of Formulae (I) and/or (II), or a pharmaceutically acceptable salt of any of the foregoing) that can be used for ameliorating and/or treating a Flaviviridae viral infection by administering to a subject an effective amount of one or more compounds described herein, or a pharmaceutically acceptable salt thereof.

Some embodiments disclosed herein relate to methods of ameliorating and/or treating a Flaviviridae viral infection that can include contacting a cell infected with the Flaviviridae virus with an effective amount of one or more compounds described herein (such as a compound of Formulae (I) and/or (II), or a pharmaceutically acceptable salt of any of the foregoing), or a pharmaceutical composition that includes one or more compounds described herein (such as a compound of Formulae (I) and/or (II), or a pharmaceutically acceptable salt of any of the foregoing). Other embodiments described herein relate to using one or more compounds described herein (such as a compound of Formulae (I) and/or (II), or a pharmaceutically acceptable salt of any of the foregoing), in the manufacture of a medicament for ameliorating and/or treating a Flaviviridae viral infection that can include contacting a cell infected with the Flaviviridae virus with an effective amount of said compound(s). Still other embodiments described herein relate to one or more compounds described herein (such as a compound of Formulae (I) and/or (II), or a pharmaceutically acceptable salt of any of the foregoing), that can be used for ameliorating and/or treating a Flaviviridae viral infection by contacting a cell infected with the Flaviviridae virus with an effective amount of said compound(s), or a pharmaceutically acceptable salt thereof.

Some embodiments disclosed herein relate to methods of inhibiting replication of a Flaviviridae virus that can include contacting a cell infected with the Flaviviridae virus with an effective amount of one or more compounds described herein (such as a compound of Formulae (I) and/or (II), or a pharmaceutically acceptable salt of any of the foregoing), or a pharmaceutical composition that includes one or more compounds described herein (such as a compound of Formulae (I) and/or (II), or a pharmaceutically acceptable salt of any of the foregoing). Other embodiments described herein relate to using one or more compounds described herein (such as a compound of Formulae (I) and/or (II), or a pharmaceutically acceptable salt of any of the foregoing), in the manufacture of a medicament for inhibiting replication of a Flaviviridae virus that can include contacting a cell infected with the Flaviviridae virus with an effective amount of said compound(s). Still other embodiments described herein relate to a compound described herein (such as a compound of Formulae (I) and/or (II), or a pharmaceutically acceptable salt of any of the foregoing), that can be used for inhibiting replication of a Flaviviridae virus by contacting a cell infected with the Flaviviridae virus with an effective amount of said compound(s), or a pharmaceutically acceptable salt thereof. In some embodiments, a polymerase of a Flaviviridae virus can be inhibited by contacting a cell infected with the Flaviviridae virus with a compound described herein (such as a compound of Formulae (I) and/or (II), or a pharmaceutically acceptable salt of any of the foregoing), and thereby, inhibit the replication of viral RNA.

HCV is an enveloped positive strand RNA virus in the Flaviviridae family. There are various nonstructural proteins of HCV, such as NS2, NS3, NS4, NS4A, NS4B, NS5A and NS5B. NS5B is believed to be an RNA-dependent RNA polymerase involved in the replication of HCV RNA.

Some embodiments disclosed herein relate to methods of ameliorating and/or treating a HCV infection that can include contacting a cell infected with HCV with an effective amount of one or more compounds described herein (such as a compound of Formulae (I) and/or (II), or a pharmaceutically acceptable salt of any of the foregoing), or a pharmaceutical composition that includes one or more compounds described herein (such as a compound of Formulae (I) and/or (II), or a pharmaceutically acceptable salt of any of the foregoing). Other embodiments described herein relate to using one or more compounds described herein (such as a compound of Formulae (I) and/or (II), or a pharmaceutically acceptable salt of any of the foregoing), in the manufacture of a medicament for ameliorating and/or treating a HCV infection that can include contacting a cell infected with HCV with an effective amount of said compound(s), or a pharmaceutically acceptable salt thereof. Still other embodiments described herein relate to one or more compounds described herein (such as a compound of Formulae (I) and/or (II), or a pharmaceutically acceptable salt of any of the foregoing), that can be used for ameliorating and/or treating a HCV infection by contacting a cell infected with HCV with an effective amount of said compound(s), or a pharmaceutically acceptable salt thereof.

Some embodiments described herein relate to a method of inhibiting NS5B polymerase activity that can include contacting a cell infected with hepatitis C virus with an effective amount of a compound of Formulae (I) and/or (II), or a pharmaceutically acceptable salt of any of the foregoing. Sonic embodiments described herein relate to a method of inhibiting NS5B polymerase activity that can include administering to a subject infected with hepatitis C virus an effective amount of a compound of Formulae (I) and/or (II), or a pharmaceutically acceptable salt of any of the foregoing. In some embodiments, a compound of Formulae (I) and/or (II), or a pharmaceutically acceptable salt of any of the foregoing, can inhibit a RNA dependent RNA polymerase, and thus, inhibit the replication of HCV RNA. In some embodiments, a compound of Formulae (I) and/or (II), or a pharmaceutically acceptable salt of any of the foregoing, can inhibit a HCV polymerase (for example, NS5B polymerase)

Some embodiments described herein relate to a method of treating a condition selected from liver fibrosis, liver cirrhosis and liver cancer in a subject suffering from one or more of the aforementioned liver conditions that can include administering to the subject an effective amount of a compound or a pharmaceutical composition described herein (for example, a compound of Formulae (I) and/or (II), or a pharmaceutically acceptable salt of any of the foregoing), wherein the liver condition is caused by a HCV infection. Some embodiments described herein relate to a method of increasing liver function in a subject having a HCV infection that can include administering to the subject an effective amount of a compound or a pharmaceutical composition described herein (for example, a compound of Formulae (I) and/or (II), or a pharmaceutically acceptable salt of any of the foregoing). Also contemplated is a method for reducing or eliminating further virus-caused liver damage in a subject having an HCV infection by administering to the subject an effective amount of a compound or a pharmaceutical composition described herein (for example, a compound of Formulae (I) and/or (II), or a pharmaceutically acceptable salt of any of the foregoing). In some embodiments, this method can include slowing or halting the progression of liver disease. In other embodiments, the course of the disease can be reversed, and stasis or improvement in liver function is contemplated. In some embodiments, liver fibrosis, liver cirrhosis and/or liver cancer can be treated; liver function can be increased; virus-caused liver damage can be reduced or eliminated; progression of liver disease can be slowed or halted; the course of the liver disease can be reversed and/or liver function can be improved or maintained by contacting a cell infected with hepatitis C virus with an effective amount of a compound described herein (for example, a compound of Formulae (I) and/or (II), or a pharmaceutically acceptable salt of any of the foregoing.)

There are a variety of genotypes of HCV, and a variety of subtypes within each genotype. For example, at present it is known that there are eleven (numbered 1 through 11) main genotypes of HCV, although others have classified the genotypes as 6 main genotypes. Each of these genotypes is further subdivided into subtypes (1a-1c; 2a-2c; 3a-3b; 4a-4e; 5a; 6a; 7a-7b; 8a-8b; 9a; 10a; and 11a). In some embodiments, an effective amount of a compound of Formulae (I) and/or (II), or a pharmaceutical acceptable salt of any of the foregoing, or a pharmaceutical composition that includes an effective amount of a compound of Formulae (I) and/or (II), or a pharmaceutical acceptable salt of any of the foregoing, can be effective to treat an infection caused by at least one genotype of HCV. In some embodiments, a compound described herein (for example, a compound of Formulae (I) and/or (II), or a pharmaceutically acceptable salt of any of the foregoing) can be effective to treat an infection caused by all 11 genotypes of HCV. In some embodiments, a compound described herein (for example, a compound of Formulae (I) and/or (II), or a pharmaceutically acceptable salt of any of the foregoing) can be effective to treat an infection caused by 3 or more, 5 or more, 7 or more, or 9 or more genotypes of HCV. In some embodiments, a compound of Formulae (I) and/or (II), or a pharmaceutical acceptable salt of any of the foregoing, can be more effective against a larger number of HCV genotypes than the standard of care. In some embodiments, a compound of Formulae (I) and/or (II), or a pharmaceutically acceptable salt of any of the foregoing, can be more effective against a particular HCV genotype than the standard of care (such as genotype 1, 2, 3, 4, 5 and/or 6).

Various indicators for determining the effectiveness of a method for treating a HCV infection are known to those skilled in the art. Examples of suitable indicators include, but are not limited to, a reduction in viral load, a reduction in viral replication, a reduction in time to seroconversion (virus undetectable in patient serum), an increase in the rate of sustained viral response to therapy, a reduction of morbidity or mortality in clinical outcomes, a reduction in the rate of liver function decrease; stasis in liver function; improvement in liver function; reduction in one or more markers of liver dysfunction, including alanine transaminase, aspartate transaminase, total bilirubin, conjugated bilirubin, gamma glutamyl transpeptidase and/or other indicator of disease response. Similarly, successful therapy with an effective amount of a compound or a pharmaceutical composition described herein (for example, a compound of Formulae (I) and/or (II), or a pharmaceutically acceptable salt of any of the foregoing) can reduce the incidence of liver cancer in HCV infected subjects.

In some embodiments, an effective amount of a compound of Formulae (I) and/or (II), or a pharmaceutically acceptable salt of any of the foregoing, is an amount that is effective to reduce HCV viral titers to undetectable levels, for example, to about 100 to about 500, to about 50 to about 100, to about 10 to about 50, or to about 15 to about 25 international units/mL serum. In some embodiments, an effective amount of a compound of Formulae (I) and/or (II), or a pharmaceutically acceptable salt of any of the foregoing, is an amount that is effective to reduce HCV viral load compared to the HCV viral load before administration of the compound of Formulae (I) and/or (II), or a pharmaceutically acceptable salt of any of the foregoing. For example, wherein the HCV viral load is measured before administration of the compound of Formulae (I) and/or (II), or a pharmaceutically acceptable salt of any of the foregoing, and again after completion of the treatment regime with the compound of Formulae (I) and/or (II), or a pharmaceutically acceptable salt of any of the foregoing (for example, 1 month after completion). In some embodiments, an effective amount of a compound of Formulae (I) and/or (II), or a pharmaceutically acceptable salt of any of the foregoing, can be an amount that is effective to reduce HCV viral load to lower than about 25 international units/mL serum. In some embodiments, an effective amount of a compound of Formulae (I) and/or (II), or a pharmaceutically acceptable salt of any of the foregoing, is an amount that is effective to achieve a reduction in HCV viral titer in the serum of the subject in the range of about 1.5-log to about a 2.5-log reduction, about a 3-log to about a 4-log reduction, or a greater than about 5-log reduction compared to the viral load before administration of the compound of Formulae (I) and/or (II), or a pharmaceutically acceptable salt of any of the foregoing. For example, the HCV viral load can be measured before administration of the compound of Formulae (I) and/or (II), or a pharmaceutically acceptable salt of any of the foregoing, and again after completion of the treatment regime with the compound of Formulae (I) and/or (II), or a pharmaceutically acceptable salt of any of the foregoing (for example, 1 month after completion).

In some embodiments, a compound of Formulae (I) and/or (II), or a pharmaceutically acceptable salt of any of the foregoing, can result in at least a 1, 2, 3, 4, 5, 10, 15, 20, 25, 50, 75, 100-fold or more reduction in the replication of the hepatitis C virus relative to pre-treatment levels in a subject, as determined after completion of the treatment regime (for example 1 month after completion). In some embodiments, a compound of Formulae (I) and/or (II), or a pharmaceutically acceptable salt of any of the foregoing, can result in a reduction of the replication of the hepatitis C virus relative to pre-treatment levels in the range of about 2 to about 5 fold, about 10 to about 20 fold, about 15 to about 40 fold, or about 50 to about 100 fold. In some embodiments, a compound of Formulae (I) and/or (II), or a pharmaceutically acceptable salt of any of the foregoing, can result in a reduction of the hepatitis C virus replication in the range of 1 to 1.5 log, 1.5 log to 2 log, 2 log to 2.5 log, 2.5 to 3 log, 3 log to 3.5 log or 3.5 to 4 log more reduction of the hepatitis C virus replication compared to the reduction of the hepatitis C virus reduction achieved by pegylated interferon in combination with ribavirin, administered according to the standard of care, or may achieve the same reduction as that standard of care therapy in a shorter period of time, for example, in one month, two months, or three months, as compared to the reduction achieved after six months of standard of care therapy with ribavirin and pegylated interferon.

In some embodiments, an effective amount of a compound of Formulae (I) and/or (II), or a pharmaceutically acceptable salt of any of the foregoing, is an amount that is effective to achieve a sustained viral response, for example, non-detectable or substantially non-detectable HCV RNA (e.g., less than about 500, less than about 200, less than about 100, less than about 25, or less than about 15 international units per milliliter serum) is found in the subject's serum for a period of at least about one month, at least about two months, at least about three months, at least about four months, at least about five months, or at least about six months following cessation of therapy.

In some embodiments, an effective amount of a compound of Formulae (I) and/or (II), or a pharmaceutically acceptable salt of any of the foregoing, can reduce a level of a marker of liver fibrosis by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80% or more, compared to the level of the marker in an untreated subject, or to a placebo-treated subject. Methods of measuring serum markers are known to those skilled in the art and include immunological-based methods, e.g., enzyme-linked immunosorbent assays (ELISA), radioimmunoassays and the like, using antibody specific for a given serum marker. A non-limiting list of examples of markers includes measuring the levels of serum alanine aminotransferase (ALT), aspartate aminotransferase (AST), alkaline phosphatase (ALP), gamma-glutamyl transpeptidase (GGT) and total bilirubin (TBIL) using known methods. In general, an ALT level of less than about 45 IU/L (international units/liter), an AST in the range of 10-34 IU/L, ALP in the range of 44-147 IU/L, GGT in the range of 0-51 IU/L, TBIL in the range of 0.3-1.9 mg/dL is considered normal. In some embodiments, an effective amount of a compound of Formulae (I) and/or (II), or a pharmaceutically acceptable salt of any of the foregoing, can be an amount effective to reduce ALT, AST, ALP, GGT and/or TBIL levels to with what is considered a normal level.

Subjects who are clinically diagnosed with HCV infection include “naive” subjects (e.g., subjects not previously treated for HCV, particularly those who have not previously received IFN-alpha-based and/or ribavirin-based therapy) and individuals who have failed prior treatment for HCV (“treatment failure” subjects). Treatment failure subjects include “non-responders” (i.e., subjects in whom the HCV titer was not significantly or sufficiently reduced by a previous treatment for HCV (≤0.5 log IU/mL), for example, a previous IFN-alpha monotherapy, a previous IFN-alpha and ribavirin combination therapy, or a previous pegylated IFN-alpha and ribavirin combination therapy); and “relapsers” (i.e., subjects who were previously treated for HCV, for example, who received a previous IFN-alpha monotherapy, a previous IFN-alpha and ribavirin combination therapy, or a previous pegylated IFN-alpha and ribavirin combination therapy, whose HCV titer decreased, and subsequently increased).

In some embodiments, a compound of Formulae (I) and/or (II), or a pharmaceutically acceptable salt of any of the foregoing, can be administered to a treatment failure subject suffering from HCV. In some embodiments, a compound of Formulae (I) and/or (II), or a pharmaceutically acceptable salt of any of the foregoing, can be administered to a non-responder subject suffering from HCV. In some embodiments, a compound of Formulae (I) and/or (II), or a pharmaceutically acceptable salt of any of the foregoing, can be administered to a relapsed subject suffering from HCV.

After a period of time, infectious agents can develop resistance to one or more therapeutic agents. The term “resistance” as used herein refers to a viral strain displaying a delayed, lessened and/or null response to a therapeutic agent(s). For example, after treatment with an antiviral agent, the viral load of a subject infected with a resistant virus may be reduced to a lesser degree compared to the amount in viral load reduction exhibited by a subject infected with a non-resistant strain. In some embodiments, a compound of Formulae (I) and/or (II), or a pharmaceutically acceptable salt of any of the foregoing, can be administered to a subject infected with an HCV strain that is resistant to one or more different anti-HCV agents (for example, an agent used in a conventional standard of care). In some embodiments, development of resistant HCV strains is delayed when a subject is treated with a compound of Formulae (I) and/or (II), or a pharmaceutically acceptable salt of any of the foregoing, compared to the development of HCV strains resistant to other HCV drugs (such as an agent used in a conventional standard of care)

In some embodiments, an effective amount of a compound of Formulae (I) and/or (II), or a pharmaceutically acceptable salt of any of the foregoing, can be administered to a subject for whom other anti-HCV medications are contraindicated. For example, administration of pegylated interferon alpha in combination with ribavirin is contraindicated in subjects with hemoglobinopathies, thalassemia major, sickle-cell anemia) and other subjects at risk from the hematologic side effects of current therapy. In some embodiments, a compound of Formulae (I) and/or (II), or a pharmaceutically acceptable salt of any of the foregoing, can be provided to a subject that is hypersensitive to interferon and/or ribavirin.

Some subjects being treated for HCV experience a viral load rebound. The term “viral load rebound” as used herein refers to a sustained ≥0.5 log IU/mL increase of viral load above nadir before the end of treatment, where nadir is a ≥0.5 log IU/mL decrease from baseline. In some embodiments, a compound of Formulae (I) and/or (II), or a pharmaceutically acceptable salt of any of the foregoing, can be administered to a subject experiencing viral load rebound, or can prevent such viral load rebound when used to treat the subject.

The standard of care for treating HCV has been associated with several side effects (adverse events). In some embodiments, a compound of Formulae (I) and/or (II), or a pharmaceutically acceptable salt of any of the foregoing, can decrease the number and/or severity of side effects that can be observed in HCV patients being treated with ribavirin and pegylated interferon according to the standard of care. Examples of side effects include, but are not limited to fever, malaise, tachycardia, chills, headache, arthralgias, myalgias, fatigue, apathy, loss of appetite, nausea, vomiting, cognitive changes, asthenia, drowsiness, lack of initiative, irritability, confusion, depression, severe depression, suicidal ideation, anemia, low white blood cell counts and thinning of hair. In some embodiments, a compound of Formulae (I) and/or (II), or a pharmaceutically acceptable salt of any of the foregoing, can be provided to a subject that discontinued a HCV therapy because of one or more adverse effects or side effects associated with one or more other HCV agents (for example, an agent used in a conventional standard of care

In some embodiments, a compound described herein (for example, a compound of Formulae (I) and/or (II), or a pharmaceutical acceptable salt of any of the foregoing) can be ameliorate and/or treat a Flavivirus infection. In some embodiments, a compound described herein (for example, a compound of Formulae (I) and/or (II), or a pharmaceutical acceptable salt of any of the foregoing) can inhibit replication of a Flavivirus.

In some embodiments, the Flavivirus can be a West Nile virus. A West Nile infection can lead to West Nile fever or severe West Nile disease (also called West Nile encephalitis or meningitis or West Nile poliomyelitis). Symptoms of West Nile fever include fever, headache, tiredness, body aches, nausea, vomiting, a skin rash (on the trunk of the body) and swollen lymph glands. Symptoms of West Nile disease include headache, high fever, neck stiffness, stupor, disorientation, coma, tremors, convulsions, muscle weakness and paralysis. Current treatment for a West Nile virus infection is supportive, and no vaccination is currently available for humans.

In some embodiments, a compound described herein (for example, a compound of Formulae (I) and/or (II), or a pharmaceutical acceptable salt of any of the foregoing) can treat and/or ameliorate an infection caused by a dengue virus, such as DENV-1, DENV-2, DENV-3 and DENV-4. A dengue virus infection can cause dengue hemorrhagic fever and/or dengue shock syndrome. In some embodiments, a compound described herein (for example, a compound of Formulae (I) and/or (II), or a pharmaceutical acceptable salt of any of the foregoing) can treat and/or ameliorate dengue hemorrhagic fever and/or dengue shock syndrome. According to the World Health Organization (WHO), global incidence of dengue has grown dramatically in recent decades. To date, there is no treatment for a dengue virus infection. Further, recovery from an infection of one serotype of dengue virus provides only partial and temporary immunity against the other serotypes. Subsequent infection(s) with another serotypes increases the likelihood of developing severe dengue (previously known as dengue hemorrhagic fever). A dengue infection is suspected with a high fever (approx. 104° F.) accompanied by one or more of the following symptoms: severe headache, pain behind the eyes, muscle and joint pain, nausea, vomiting, swollen glands and rash.

Yellow fever is an acute viral hemorrhagic disease. As provided by the WHO, up to 50% of severely affected persons without treatment die from yellow fever. An estimated 200,000 cases of yellow fever, causing 30,000 deaths, worldwide occur each year. As with other Flaviviruses, there is no cure or specific treatment for yellow fever, and treatment with ribavirin and interferons are insufficient. In some embodiments, the Flavivirus can be yellow fever virus. Symptoms of a yellow fever infection include fever, muscle pain with prominent backache, headache, shivers, loss of appetite, nausea, vomiting, jaundice and bleeding (for example from the mouth, nose, eyes and/or stomach).

In yet still other embodiments, the Flavivirus can be an encephalitis virus from within the Flavivirus genus. Examples of encephalitis viruses include, but are not limited to, Japanese encephalitis virus, St. Louis encephalitis virus and tick borne encephalitis. Viral encephalitis causes inflammation of the brain and/or meninges. Symptoms include high fever, headache, sensitivity to light, stiff neck and back, vomiting, confusion, seizures, paralysis and coma. There is no specific treatment for an encephalitis infection, such as Japanese encephalitis, St. Louis encephalitis and tick borne encephalitis.

In some embodiments, the Flavivirus can be a Zika virus. According to the Centers for Disease Control, Zika is spread mostly by the bite of an infected Aedes species mosquito (Ae. aegypti and Ae. albopictus) and can be passed from a pregnant woman to her fetus. Infection during pregnancy can cause certain birth defects. Many people infected with Zika virus will not have symptoms or will only have mild symptoms. The most common symptoms of Zika are fever, rash, joint pain and conjunctivitis. Zika is usually mild with symptoms lasting for several days to a week. People usually do not get sick enough to go to the hospital, and they very rarely die of Zika. For this reason, many people might not realize they have been infected. Symptoms of Zika are similar to other viruses spread through mosquito bites, like dengue and chikungunya. In some embodiments, a compound described herein (for example, a compound of Formulae (I) and/or (II), or a pharmaceutically acceptable salt of any of the foregoing) can be provided prophylactically to a subject through administration and/or contact with a cell in the subject, wherein when the subject is infected with the Zika virus, the subject has an immunity to the Zika virus and/or develops a Zika virus infection that is less severe compared to the Zika infection in a subject that did not prophylactically receive a compound described herein.

Various indicators for determining the effectiveness of a method for treating an Picornaviridae and/or Flaviviridae viral infection are known to those skilled in the art. Example of suitable indicators include, but are not limited to, a reduction in viral load, a reduction in viral replication, a reduction in time to seroconversion (virus undetectable in patient serum), a reduction of morbidity or mortality in clinical outcomes, and/or other indicator(s) of disease response. Further indicators include one or more overall quality of life health indicators, such as reduced illness duration, reduced illness severity, reduced time to return to normal health and normal activity and reduced time to alleviation of one or more symptoms. In some embodiments, a compound of Formulae (I) and/or (II), or a pharmaceutically acceptable salt of any of the foregoing, can result in the reduction, alleviation or positive indication of one or more of the aforementioned indicators compared to a subject who is receiving the standard of care (for HCV) or an untreated subject (Picornaviridae, and other Flaviviridae viral infections besides HCV). Effects/symptoms of a Picornaviridae viral infection are described herein, and include, but are not limited to, fever, blisters, rash, meningitis, conjunctivitis, acute hemorrhagic conjunctivitis (AHC), sore throat, nasal congestion, runny nose, sneezing, coughing, loss of appetite, muscle aches, headache, fatigue, nausea, jaundice, encephalitis, herpangina, myocarditis, pericarditis, meningitis, Bornholm disease, myalgia, nasal congestion, muscle weakness, loss of appetite, fever, vomiting, abdominal pain, abdominal discomfort, dark urine and muscle pain. Effects/symptoms of a Flaviviridae viral infection are also described herein.

In some embodiments, a compound of Formulae (I) and/or (II), or a pharmaceutically acceptable salt of any of the foregoing, can result in a reduction in the length and/or severity of one or more symptoms associated with a Picornaviridae or a Flaviviridae viral infection compared to a subject who is receiving the standard of care (for HCV) or an untreated subject (Picornaviridae, and other Flaviviridae viral infection besides HCV). Table 1 provides some embodiments of the percentage improvements obtained using a compound of Formulae (I) and/or (II), or a pharmaceutically acceptable salt of any of the foregoing, as compared to the standard of care (for HCV) or an untreated subject (Picornaviridae, and other Flaviviridae viral infection besides HCV). Examples include the following: in some embodiments, a compound of Formulae (I) and/or (II), or a pharmaceutically acceptable salt of any of the foregoing, results in a percentage of non-responders that is 10% less than the percentage of non-responders receiving the standard of care for HCV; in some embodiments, a compound of Formulae (I) and/or (II), or a pharmaceutically acceptable salt of any of the foregoing, results in a duration of illness that is in the range of about 10% to about 30% less than compared to the duration of illness experienced by a subject who is untreated for a Zika viral infection; and in some embodiments, a compound of Formulae (I) and/or (II), or a pharmaceutically acceptable salt of any of the foregoing, results in a severity of a symptom (such as one of those described herein) that is 25% less than compared to the severity of the same symptom experienced by a subject who is untreated for a dengue virus infection. Methods of quantifying the severity of a side effect and/or symptom are known to those skilled in the art.

TABLE 1

| Percentage | | Percentage | Percentage | | |
|---|---|---|---|---|---|
| of non- | Percentage | of | of viral load | Number of | Severity of |
| responders | of relapsers | resistance | rebound | side effects | side effect(s) |
| 10% less | 10% less | 10% less | 10% less | 10% less | 10% less |
| 25% less | 25% less | 25% less | 25% less | 25% less | 25% less |
| 40% less | 40% less | 40% less | 40% less | 40% less | 40% less |
| 50% less | 50% less | 50% less | 50% less | 50% less | 50% less |
| 60% less | 60% less | 60% less | 60% less | 60% less | 60% less |
| 70% less | 70% less | 70% less | 70% less | 70% less | 70% less |
| 80% less | 80% less | 80% less | 80% less | 80% less | 80% less |
| 90% less | 90% less | 90% less | 90% less | 90% less | 90% less |
| about 10% | about 10% | about 10% | about 10% to | about 10% to | about 10% to |
| to about | to about | to about | about 30% | about 30% | about 30% |
| 30% less | 30% less | 30% less | less | less | less |
| about 20% | about 20% | about 20% | about 20% to | about 20% to | about 20% to |
| to about | to about | to about | about 50% | about 50% | about 50% |
| 50% less | 50% less | 50% less | less | less | less |
| about 30% | about 30% | about 30% | about 30% to | about 30% to | about 30% to |
| to about | to about | to about | about 70% | about 70% | about 70% |
| 70% less | 70% less | 70% less | less | less | less |
| about 20% | about 20% | about 20% | about 20% to | about 20% to | about 20% to |
| to about | to about | to about | about 80% | about 80% | about 80% |
| 80% less | 80% less | 80% less | less | less | less |
| Duration of | Duration of | Duration of | Severity of | Severity of | Severity of |
| illness | illness | illness | symptom(s) | symptom(s) | symptom(s) |
| 10% less | 60% less | about 10% | 10% less | 60% less | about 10% to |
| | | to about | | | about 30% |
| | | 30% less | | | less |
| 25% less | 70% less | about 20% | 25% less | 70% less | about 20% to |
| | | to about | | | about 50% |
| | | 50% less | | | less |
| 40% less | 80% less | about 30% | 40% less | 80% less | about 30% to |
| | | to about | | | about 70% |
| | | 70% less | | | less |
| 50% less | 90% less | about 20% | 50% less | 90% less | about 20% to |
| | | to about | | | about 80% |
| | | 80% less | | | less |

In some embodiments, the compound can be a compound of Formulae (I) and/or (II), or a pharmaceutical acceptable salt of any of the foregoing, wherein R^{1A} is hydrogen or deuterium. In other embodiments, the compound can be a compound of Formulae (I) and/or (II), wherein compound of Formulae (I) and/or (II) is a mono, di, or triphosphate, or a pharmaceutically acceptable salt of any of the foregoing. In still other embodiments, the compound can be a compound of Formulae (I) and/or (II), wherein compound of Formulae (I) and/or (II) is a thiomonophosphate, alpha-thiodiphosphate, or alpha-thiotriphosphate, or a pharmaceutically acceptable salt of any of the foregoing. In some embodiments, the compound of Formulae (I) and/or (II), or a pharmaceutical acceptable salt of any of the foregoing, that can be used to ameliorate and/or treat a Picornaviridae viral infection (and/or a Flaviviridae viral infection) and/or inhibit replication of a Picornaviridae virus (and/or a Flaviviridae virus) can be any of the embodiments provided in any of the embodiments described herein.

As used herein, a “subject” refers to an animal that is the object of treatment, observation or experiment. “Animal” includes cold- and warm-blooded vertebrates and invertebrates such as fish, shellfish, reptiles and, in particular, mammals. “Mammal” includes, without limitation, mice, rats, rabbits, guinea pigs, dogs, cats, sheep, goats, cows, horses, primates, such as monkeys, chimpanzees and apes, and, in particular, humans. In some embodiments, the subject is human.

As used herein, the terms “treating,” “treatment,” “therapeutic,” or “therapy” do not necessarily mean total cure or abolition of the disease or condition. Any alleviation of any undesired signs or symptoms of a disease or condition, to any extent can be considered treatment and/or therapy. Furthermore, treatment may include acts that may worsen the patient's overall feeling of well-being or appearance.

The terms “therapeutically effective amount” and “effective amount” are used to indicate an amount of an active compound, or pharmaceutical agent, that elicits the biological or medicinal response indicated. For example, an effective amount of compound can be the amount needed to prevent, alleviate or ameliorate symptoms of disease or prolong the survival of the subject being treated This response may occur in a tissue, system, animal or human and includes alleviation of the signs or symptoms of the disease being treated. Determination of an effective amount is well within the capability of those skilled in the art, in view of the disclosure provided herein. The effective amount of the compounds disclosed herein required as a dose will depend on the route of administration, the type of animal, including human, being treated, and the physical characteristics of the specific animal under consideration. The dose can be tailored to achieve a desired effect, but will depend on such factors as weight, diet, concurrent medication and other factors which those skilled in the medical arts will recognize.

As will be readily apparent to one skilled in the art, the useful in vivo dosage to be administered and the particular mode of administration will vary depending upon the age, weight, the severity of the affliction, and mammalian species treated, the particular compounds employed, and the specific use for which these compounds are employed. The determination of effective dosage levels, that is the dosage levels necessary to achieve the desired result, can be accomplished by one skilled in the art using routine methods, for example, human clinical trials and in vitro studies.

The dosage may range broadly, depending upon the desired effects and the therapeutic indication. Alternatively dosages may be based and calculated upon the surface area of the patient, as understood by those of skill in the art. Although the exact dosage will be determined on a drug-by-drug basis, in most cases, some generalizations regarding the dosage can be made. The daily dosage regimen for an adult human patient may be, for example, an oral dose of between 0.01 mg and 3000 mg of each active ingredient, preferably between 1 mg and 700 mg, e.g., 5 to 200 mg. The dosage may be a single one or a series of two or more given in the course of one or more days, as is needed by the subject. In some embodiments, the compounds will be administered for a period of continuous therapy, for example for a week or more, or for months or years. In some embodiments, a compound of Formulae (I) and/or (II), or a pharmaceutically acceptable salt of any of the foregoing, can be administered less frequently compared to the frequency of administration of an agent within the standard of care. In some embodiments, a compound of Formulae (I) and/or (II), or a pharmaceutically acceptable salt of any of the foregoing, can be administered one time per day. For example, a compound of Formulae (I) and/or (II), or a pharmaceutically acceptable salt of any of the foregoing, can be administered one time per day to a subject suffering from a picornavirus infection. In some embodiments, the total time of the treatment regime with a. compound of Formulae (I) and/or (II), or a pharmaceutically acceptable salt of any of the foregoing, can be less compared to the total time of the treatment regime with the standard of care.

In instances where human dosages for compounds have been established for at least some condition, those same dosages may be used, or dosages that are between about 0.1% and 500%, more preferably between about 25% and 250% of the established human dosage. Where no human dosage is established, as will be the case for newly-discovered pharmaceutical compositions, a suitable human dosage can be inferred from ED₅₀ or ID₅₀ values, or other appropriate values derived from in vitro or in vivo studies, as qualified by toxicity studies and efficacy studies in animals.

In cases of administration of a pharmaceutically acceptable salt, dosages may be calculated as the free base. As will be understood by those of skill in the art, in certain situations it may be necessary to administer the compounds disclosed herein in amounts that exceed, or even far exceed, the above-stated, preferred dosage range in order to effectively and aggressively treat particularly aggressive diseases or infections.

Dosage amount and interval may be adjusted individually to provide plasma levels of the active moiety which are sufficient to maintain the modulating effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations. Dosage intervals can also be determined using MEC value. Compositions should be administered using a regimen which maintains plasma levels above the MFC for 10-90% of the time, preferably between 30-90% and most preferably between 50-90%. In cases of local administration or selective uptake, the effective local concentration of the drug may not be related to plasma concentration.

It should be noted that the attending physician would know how to and When to terminate, interrupt, or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the condition to be treated and to the route of administration. The severity of the condition may, for example, be evaluated, in part, by standard prognostic evaluation methods. Further, the dose and perhaps dose frequency, will also vary according to the age, body weight and response of the individual patient. A program comparable to that discussed above may be used in veterinary medicine.

Compounds disclosed herein can be evaluated for efficacy and toxicity using known methods. For example, the toxicology of a particular compound, or of a subset of the compounds, sharing certain chemical moieties, may be established by determining in vitro toxicity towards a cell line, such as a mammalian, and preferably human, cell line. The results of such studies are often predictive of toxicity in animals, such as mammals, or more specifically, humans. Alternatively, the toxicity of particular compounds in an animal model, such as mice, rats, rabbits, or monkeys, may be determined using known methods. The efficacy of a particular compound may be established using several recognized methods, such as in vitro methods, animal models, or human clinical trials. When selecting a model to determine efficacy, the skilled artisan can be guided by the state of the art to choose an appropriate model, dose, route of administration and/or regime.

### Combination Therapies

In some embodiments, the compounds disclosed herein, such as a compound of Formulae (I) and/or (II), or a pharmaceutically acceptable salt of any of the foregoing, or a pharmaceutical composition that includes a compound described herein, or a pharmaceutically acceptable salt thereof, can be used in combination with one or more additional agent(s) for treating, ameliorating and/or inhibiting a Picornaviridae and/or Flaviviridae viral infection.

In some embodiments, a compound of Formulae (I) and/or (II), or a pharmaceutically acceptable salt of any of the foregoing, can be administered with one or more additional agent(s) together in a single pharmaceutical composition. In some embodiments, a compound of Formulae (I) and/or (II), or a pharmaceutically acceptable salt of any of the foregoing, can be administered with one or more additional agent(s) as two or more separate pharmaceutical compositions. For example, a compound of Formulae (I) and/or (II), or a pharmaceutically acceptable salt of any of the foregoing, can be administered in one pharmaceutical composition, and at least one of the additional agents can be administered in a second pharmaceutical composition. If there are at least two additional agents, one or more of the additional agents can be in a first pharmaceutical composition that includes a compound of Formulae (I) and/or (II), or a pharmaceutically acceptable salt of any of the foregoing, and at least one of the other additional agent(s) can be in a second pharmaceutical composition.

The dosing amount(s) and dosing schedule(s) when using a compound of Formulae (I) and/or (II), or a pharmaceutically acceptable salt of any of the foregoing, or a pharmaceutical composition that includes a compound of Formulae (I) and/or (II), or a pharmaceutically acceptable salt of any of the foregoing, and one or more additional agents are within the knowledge of those skilled in the art. For example, when performing a conventional standard of care therapy using art-recognized dosing amounts and dosing schedules, a compound of Formulae (I) and/or (II), or a pharmaceutically acceptable salt of any of the foregoing, or a pharmaceutical composition that includes a compound of Formulae (I) and/or (II), or a pharmaceutically acceptable salt of any of the foregoing, can be administered in addition to that therapy, or in place of one of the agents of a combination therapy, using effective amounts and dosing protocols as described herein.

The order of administration of a compound of Formulae (I) and/or (II), or a pharmaceutically acceptable salt of any of the foregoing, with one or more additional agent(s) can vary. In some embodiments, a compound of Formulae (I) and/or (II), or a pharmaceutically acceptable salt of any of the foregoing, can be administered prior to all additional agents. In other embodiments, a compound of Formulae (I) and/or (II), or a pharmaceutically acceptable salt of any of the foregoing, can be administered prior to at least one additional agent. In still other embodiments, a compound of Formulae (I) and/or (II), or a pharmaceutically acceptable salt of any of the foregoing, can be administered concomitantly with one or more additional agent(s). In yet still other embodiments, a compound of Formulae (I) and/or (II), or a pharmaceutically acceptable salt of any of the foregoing, can be administered subsequent to the administration of at least one additional agent. In some embodiments, a compound of Formulae (I) and/or (II), or a pharmaceutically acceptable salt of any of the foregoing, can be administered subsequent to the administration of all additional agents.

In some embodiments, the combination of a compound of Formulae (I) and/or (II), or a pharmaceutically acceptable salt of any of the foregoing, in combination with one or more additional agent(s) can result in an additive effect. In some embodiments, the combination of a compound of Formulae (I) and/or (II), or a pharmaceutically acceptable salt of any of the foregoing, used in combination with one or more additional agent(s) can result in a synergistic effect. In some embodiments, the combination of a compound of Formulae (I) and/or (II), or a pharmaceutically acceptable salt of any of the foregoing, used in combination with one or more additional agent(s) can result in a strongly synergistic effect. In some embodiments, the combination of a compound of Formulae (I) and/or (II), or a pharmaceutically acceptable salt of any of the foregoing, in combination with one or more additional agent(s) is not antagonistic.

As used herein, the term “antagonistic” means that the activity of the combination of compounds is less compared to the sum of the activities of the compounds in combination when the activity of each compound is determined individually (i.e., as a single compound). As used herein, the term “synergistic effect” means that the activity of the combination of compounds is greater than the sum of the individual activities of the compounds in the combination when the activity of each compound is determined individually. As used herein, the term “additive effect” means that the activity of the combination of compounds is about equal to the sum of the individual activities of the compound in the combination when the activity of each compound is determined individually.

A potential advantage of utilizing a compound of Formulae (I) and/or (II), or a pharmaceutically acceptable salt of any of the foregoing, in combination with one or more additional agent(s) may be a reduction in the required amount(s) of one or more additional agent(s) that is effective in treating a picornavirus virus infection, as compared to the amount required to achieve same therapeutic result when one or more additional agent(s) are administered without a compound of Formulae (I) and/or (II), or a pharmaceutically acceptable salt of any of the foregoing. Another potential advantage of utilizing a compound of Formulae (I) and/or (II), or a pharmaceutically acceptable salt of any of the foregoing, in combination with one or more additional agent(s) is that the use of two or more compounds having different mechanism of actions can create a higher barrier to the development of resistant viral strains compared to the barrier when a compound is administered as monotherapy.

Additional advantages of utilizing a compound of Formulae (I) and/or (II), or a pharmaceutically acceptable salt of any of the foregoing, in combination with one or more additional agent(s) may include little to no cross resistance between a compound of Formulae (I) and/or (II), or a pharmaceutically acceptable salt of any of the foregoing, and one or more additional agent(s) thereof; different routes for elimination of a compound of Formulae (I) and/or (II), or a pharmaceutically acceptable salt of any of the foregoing, and one or more additional agent(s); little to no overlapping toxicities between a compound of Formulae (I) and/or (II), or a pharmaceutically acceptable salt of any of the foregoing, and one or more additional agent(s); little to no significant effects on cytochrome P450; little to no pharmacokinetic interactions between a compound of Formulae (I) and/or (II), or a pharmaceutically acceptable salt of any of the foregoing, and one or more additional agent(s); greater percentage of subjects achieving a sustained viral response compared to when a compound is administered as monotherapy and/or a decrease in treatment time to achieve a sustained viral response compared to when a compound is administered as monotherapy; and reduction in the amount of the one or more additional agent(s) administered to subjects when administered with a compound of Formulae (I) and/or (II), or a pharmaceutically acceptable salt of any of the foregoing, compared to when the one or more additional agent(s) is administered as monotherapy.

For treating of a Picornaviridae and/or a Flaviviridae viral infection other than HCV, examples of additional agents that can be used in combination with a compound of Formulae (I) and/or (II), or a pharmaceutically acceptable salt of any of the foregoing, or a pharmaceutical composition that includes a compound of Formulae (I) and/or (II), or a pharmaceutically acceptable salt of any of the foregoing, include, but are not limited to, ribavirin and an interferon (including those described herein).

For the treatment of HCV, examples of additional agents that can be used in combination with a compound of Formulae (I) and/or (II), or a pharmaceutically acceptable salt of any of the foregoing, or a pharmaceutical composition that includes a compound of Formulae (I) and/or (II), or a pharmaceutically acceptable salt of any of the foregoing, include, but are not limited to, agents currently used in a conventional standard of care for treating HCV, HCV protease inhibitors, HCV polymerase inhibitors, NS5A inhibitors, other antiviral compounds, compounds of Formula (AA), (including pharmaceutically acceptable salts and pharmaceutical compositions that can include a compound of Formula (AA), or a pharmaceutically acceptable salt thereof), compounds of Formula (BB) (including pharmaceutically acceptable salts and pharmaceutical compositions that can include a compound of Formula (BB), or a pharmaceutically acceptable salt thereof), compounds of Formula (CC) (including pharmaceutically acceptable salts and pharmaceutical compositions that can include a compound of Formula (CC), or a pharmaceutically acceptable salt thereof), compounds of Formula (DD) (including pharmaceutically acceptable salts and pharmaceutical compositions that can include a compound of Formula (DD), or a pharmaceutically acceptable salt thereof), compounds of Formula (EE) (including pharmaceutically acceptable salts and pharmaceutical compositions that can include a compound of Formula (EE), or a pharmaceutically acceptable salt thereof), compounds of Formula (FF) (including pharmaceutically acceptable salts and pharmaceutical compositions that can include a compound of Formula (FF), or a pharmaceutically acceptable salt thereof), and/or combinations thereof. In some embodiments, a compound of Formulae (I) and/or (II), or a pharmaceutically acceptable salt of any of the foregoing, or a pharmaceutical composition that includes a compound of Formulae (I) and/or (II), or a pharmaceutically acceptable salt of any of the foregoing, can be used with one, two, three or more additional agents described herein.

In some embodiments, a compound of Formulae (I) and/or (II), or a pharmaceutically acceptable salt of any of the foregoing, or a pharmaceutical composition that includes a compound of Formulae (I) and/or (II), or a pharmaceutically acceptable salt of any of the foregoing, can be used in combination with an agent(s) currently used in a conventional standard of care therapy For example, for the treatment of HCV, a compound disclosed herein can be used in combination with Pegylated interferon-alpha-2a (brand name PEGASYS®) and ribavirin, Pegylated interferon-alpha-2b (brand name PEG-INTRON®) and ribavirin, Pegylated interferon-alpha-2a, Pegylated interferon-alpha-2b, or ribavirin,

In some embodiments, a compound of Formulae (I) and/or (II), or a pharmaceutically acceptable salt of any of the foregoing, or a pharmaceutical composition that includes a compound of Formulae (I) and/or (II), or a pharmaceutically acceptable salt of any of the foregoing, can be substituted for an agent currently used in a conventional standard of care therapy. For example, for the treatment of HCV, a compound of Formulae (I) and/or (II), or a pharmaceutically acceptable salt of any of the foregoing, or a pharmaceutical composition that includes a compound of Formulae (I) and/or (II), or a pharmaceutically acceptable salt of any of the foregoing, can be used in place of ribavirin.

In some embodiments, a compound of Formulae (I) and/or (II), or a pharmaceutically acceptable salt of any of the foregoing, or a pharmaceutical composition that includes a compound of Formulae (I) and/or (II), or a pharmaceutically acceptable salt of any of the foregoing, can be used in combination with an interferon, such as a pegylated interferon. Examples of suitable interferons include, but are not limited to, Pegylated interferon-alpha-2a (brand name PEGASYS®), Pegylated interferon-alpha-2b) (brand name PEG-INTRON®), interferon alfacon-1 (brand name INFERGEN®), pegylated interferon lambda and/or a combination thereof.

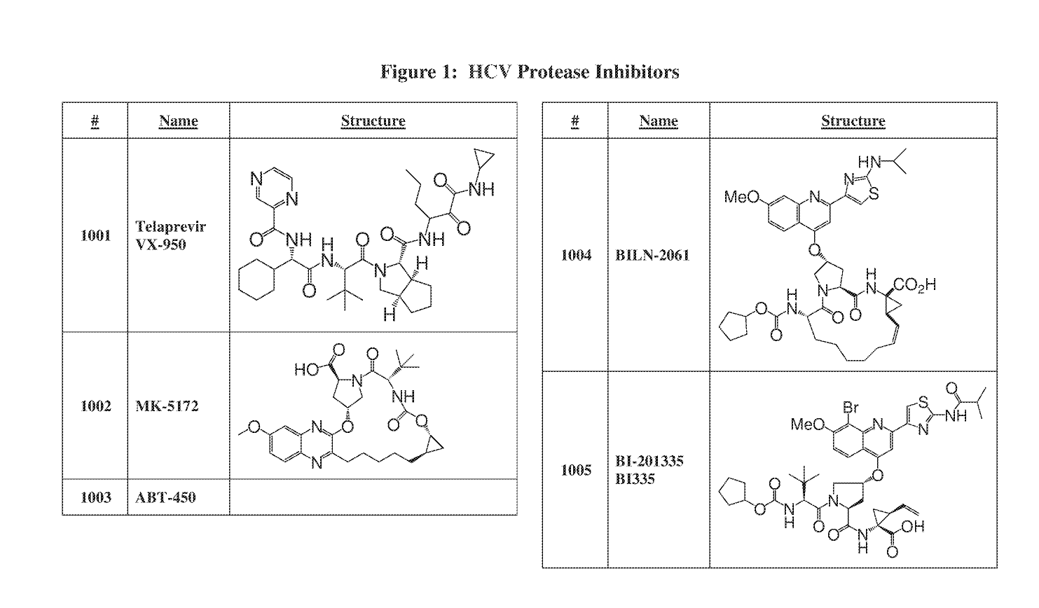

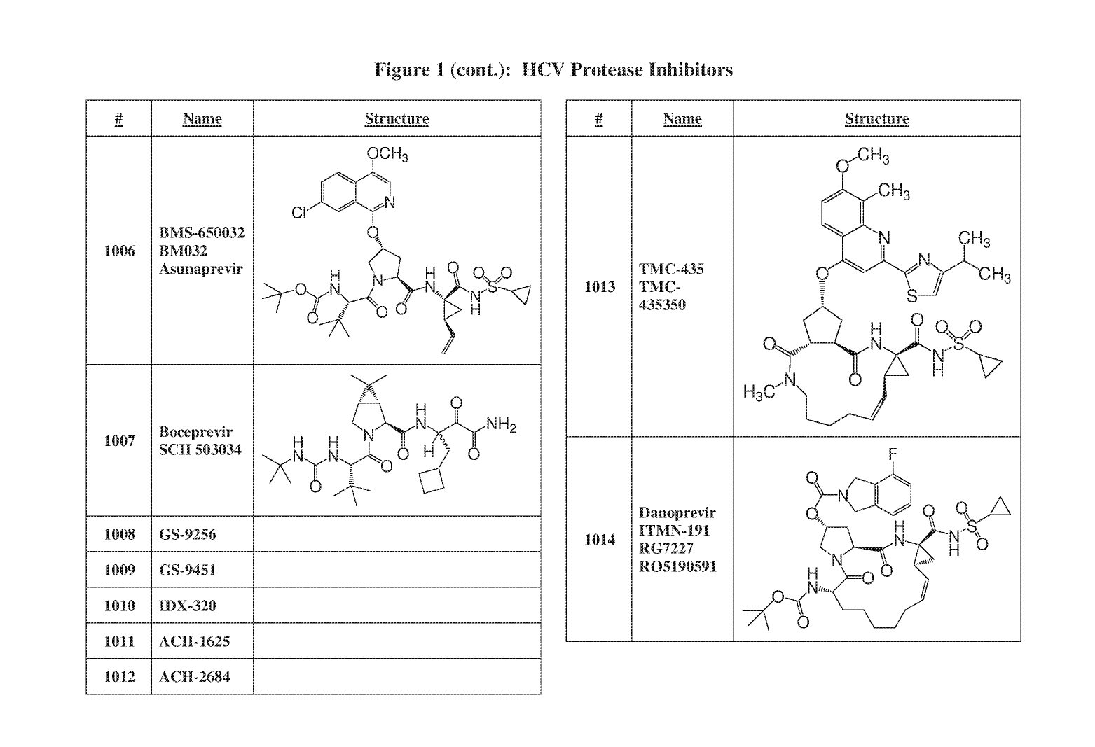

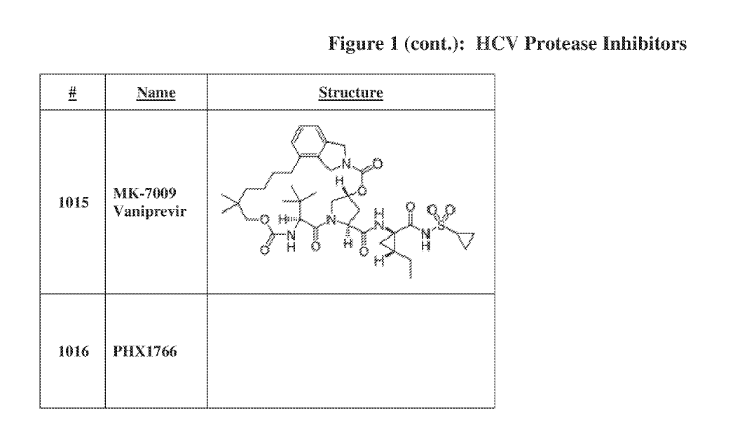

In some embodiments, a compound of Formulae (I) and/or (II), or a pharmaceutically acceptable salt of any of the foregoing, or a pharmaceutical composition that includes a compound of Formulae (I) and/or (II), or a pharmaceutically acceptable salt of any of the foregoing, can be used in combination with a HCV protease inhibitor. A non-limiting list of example HCV protease inhibitors include the following: VX-950 (TELAPREVIR®), MK-5172, ABT-450, BILN-2061, BI-201335, BMS-650032, SCH 503034 (BOCEPREVIR®), GS-9256, GS-9451, IDX-320, ACH-1625, ACH-2684, TMC-435, ITMN-191 (DANOPREVIR®) and/or a combination thereof. Additional HCV protease inhibitors suitable for use in combination with a compound of Formulae (I) and/or (II), or a pharmaceutically acceptable salt of any of the foregoing, or a pharmaceutical composition that includes a compound of Formulae (I) and/or (II), or a pharmaceutically acceptable salt of any of the foregoing, include VP-19744, PSI-879, VM-759/VX-759, HCV-371, IDX-375, GL-60667, JTK-109, PSI-6130, R1479, R-1626, R-7182, MK-0608, INX-8014, INX-8018, A-848837, A-837093, BILB-1941, VCH-916, VCH-716, GSK-71185, GSK-625433, XTL-2125 and those disclosed in PCT Publication No. WO 2012/142085, which is hereby incorporated by reference for the limited purpose of its disclosure of HCV protease inhibitors, HCV polymerase inhibitors and NS5A inhibitors. A non-limiting list of example HCV protease inhibitors includes the compounds numbered 1001-1016 in FIG. 1.

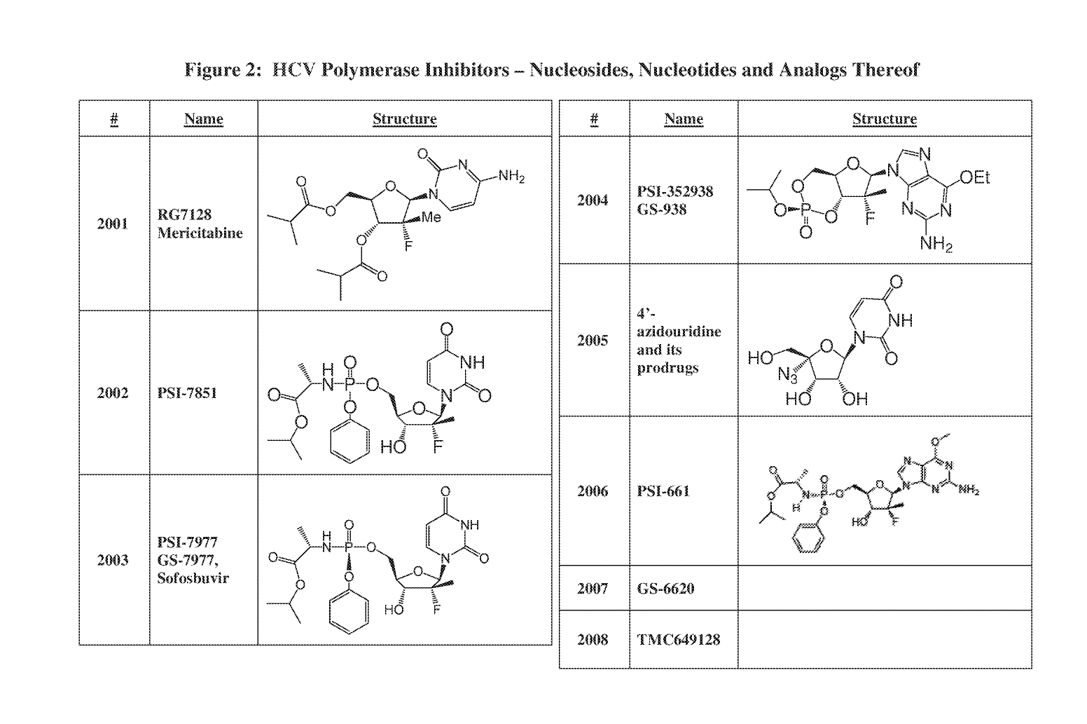

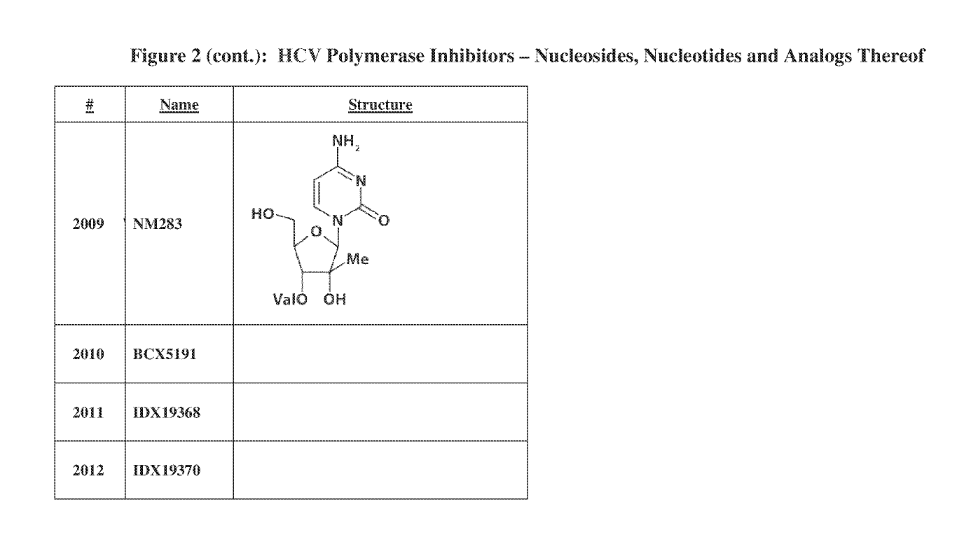

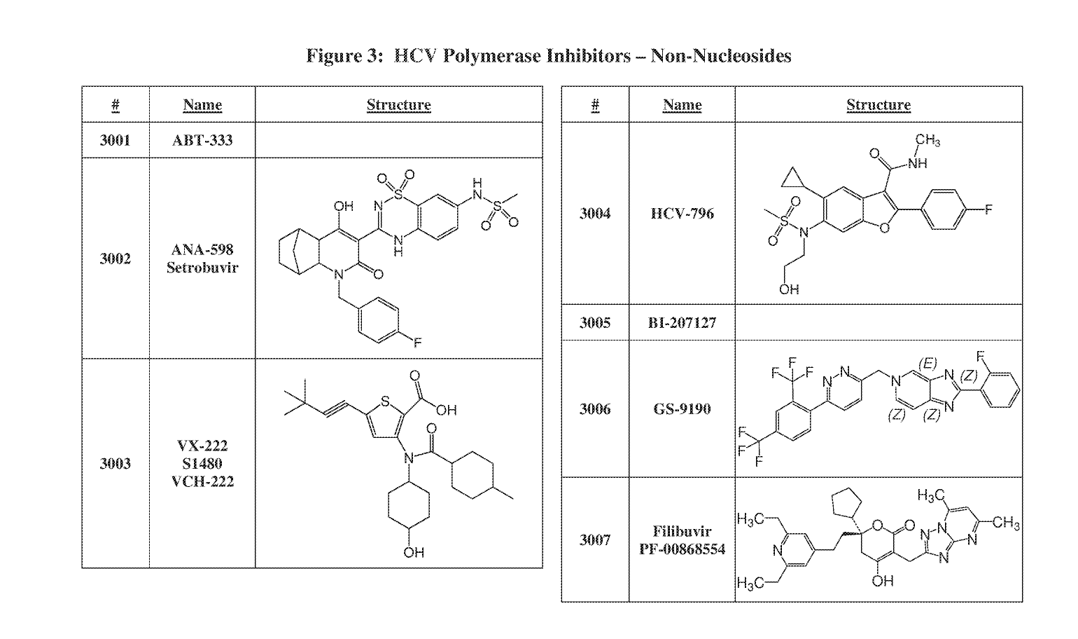

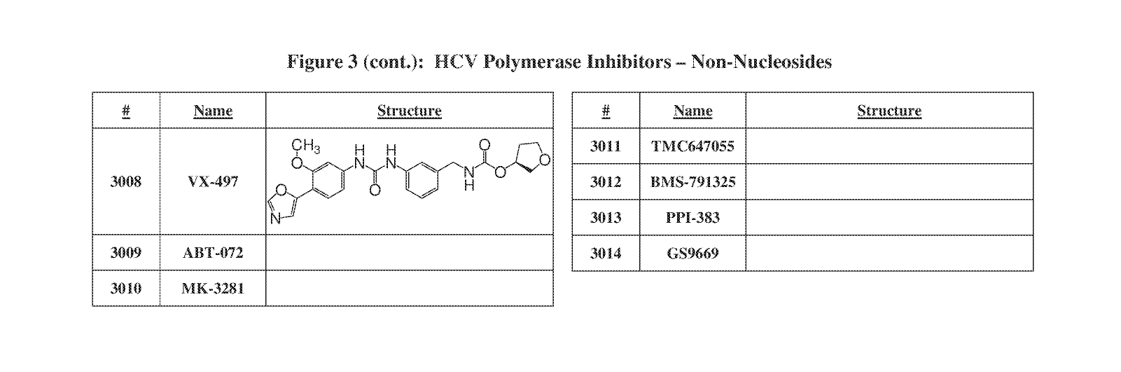

In some embodiments, a compound of Formulae (I) and/or (II), or a pharmaceutically acceptable salt of any of the foregoing, or a pharmaceutical composition that includes a compound of Formulae (I) and/or (II), or a pharmaceutically acceptable salt of any of the foregoing, can be used in combination with a HCV polymerase inhibitor. In some embodiments, the HCV polymerase inhibitor can be a nucleoside inhibitor. In other embodiments, the HCV polymerase inhibitor can be a non-nucleoside inhibitor. Examples of suitable nucleoside inhibitors include, but are not limited to, RG7128, PSI-7851, PSI-7977, INX-189, PSI-352938, PSI-661, 4′-azidouridine (including known prodrugs of 4′-azidouridine), GS-6620, MX-184 and TMC649128 and/or combinations thereof. A non-limiting list of example nucleoside inhibitors includes compounds numbered 2001-2012 in FIG. 2. Examples of suitable non-nucleoside inhibitors include, but are not limited to, ABT-333, ANA-598, VX-222, HCV-796, BI-207127, GS-9190, PF-00868554 (FILIBUVIR®), VX-497 and/or combinations thereof. A non-limiting list of example non-nucleoside inhibitors includes the compounds numbered 3001-3014 in FIG. 3. Further HCV polymnerase inhibitors suitable for use in combination with a compound of Formulae (I) and/or (II), or a pharmaceutically acceptable salt of any of the foregoing, or a pharmaceutical composition that includes a compound of Formulae (I) and/or (II), or a pharmaceutically acceptable salt of any of the foregoing, include VX-500, VX-813, VBY-376, TMC-435350, EZ-058, EZ-063, GS-9132,ACH-1095, IDX-136, IDX-316, ITMN-8356, ITMN-8347, ITMN-8096, ITMN-7587, VX-985 and those disclosed in PCT Publication No. WO 2012/142085.

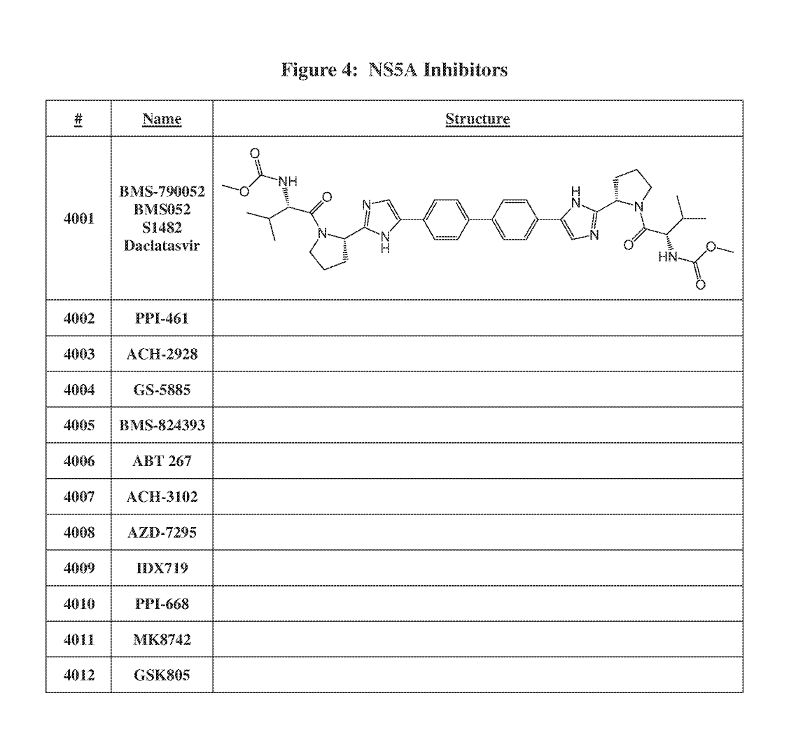

In some embodiments, a compound of Formulae (I) and/or (II), or a pharmaceutically acceptable salt of any of the foregoing, or a pharmaceutical composition that includes a compound of Formulae (I) and/or (II), or a pharmaceutically acceptable salt of any of the foregoing, can be used in combination with a NS5A inhibitor. Examples of NS5A inhibitors include BMS-790052, PPI-461, ACTT-2928, GS-5885, BMS-824393 and/or combinations thereof. A non-limiting list of example NS5A inhibitors includes the compounds numbered 4001-4012 in FIG. 4. Additional NS5A inhibitors suitable for use in combination with a compound of Formulae (I) and/or (II), or a pharmaceutically acceptable salt of any of the foregoing, or a pharmaceutical composition that includes a compound of Formulae (I) and/or (II), or a pharmaceutically acceptable salt of any of the foregoing, include A-832, PPI-1301 and those disclosed in PCI Publication No. WO 2012/142085.

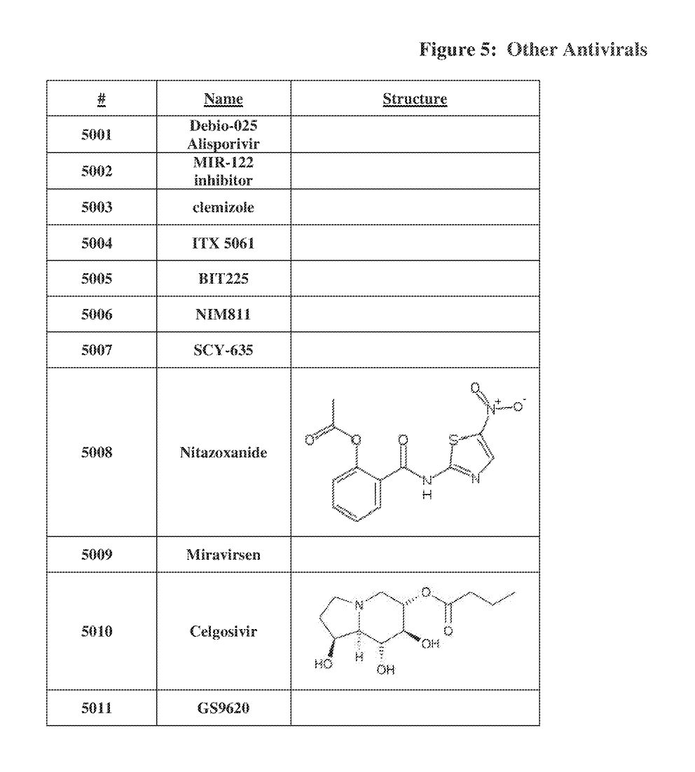

In some embodiments, a compound of Formulae (I) and/or (II), or a pharmaceutically acceptable salt of any of the foregoing, or a pharmaceutical composition that includes a compound of Formulae (I) and/or (II), or a pharmaceutically acceptable salt of any of the foregoing, can be used in combination with other antiviral compounds. Examples of other antiviral compounds include, but are not limited to, Debio-025, a MIR-122 inhibitor (for example, Miravirsen (SPC3649)), cyclosporin A and/or combinations thereof. A non-limiting list of example other antiviral compounds includes the compounds numbered 5001-5011 in FIG. 5.

For each of Formulae (AA), (BB), (CC), (DD), (EE) and (FF), or a pharmaceutically acceptable salt of any of the foregoing, each variable pertains only to each individual formula. For example for Compounds of Formula (AA), the variables described under Compounds of Formula (AA) refer only to Compounds of Formula (AA) and not Compounds of Formula (BB) or any of the other formulae provided in this combination therapy section, unless stated otherwise.

In some embodiments, a compound of Formulae (I) and/or (II), or a pharmaceutically acceptable salt of any of the foregoing, or a pharmaceutical composition that includes a compound of Formulae (I) and/or (II), or a pharmaceutically acceptable salt of any of the foregoing, can be used in combination with a compound of Formula (AA), or a pharmaceutically acceptable salt thereof, or a pharmaceutical composition that includes a compound of Formula (AA), or a pharmaceutically acceptable salt thereof (see, U.S. Publication No. 2013/0164261 A1, filed Dec. 20, 2012, the contents of which are incorporated by reference in its entirety):

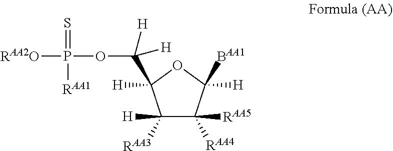

wherein: B^{AA1} can be an optionally substituted heterocyclic base or an optionally substituted heterocyclic base with a protected amino group; R^{AA1} can be selected from O⁻, OH, an optionally substituted N-linked amino acid and an optionally substituted N-linked amino acid ester derivative; R^{AA2} can be absent or selected from hydrogen, an optionally substituted aryl, an optionally substituted heteroaryl, an optionally substituted heterocyclyl and

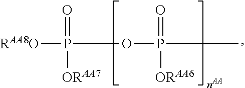

wherein R^{AA6}, R^{AA7} and R^{AA8} can be independently absent or hydrogen and n^{AA} can be 0 or 1; provided that when R^{AA1} is O⁻ or OH, then R^{AA2} is absent, hydrogen or

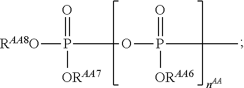

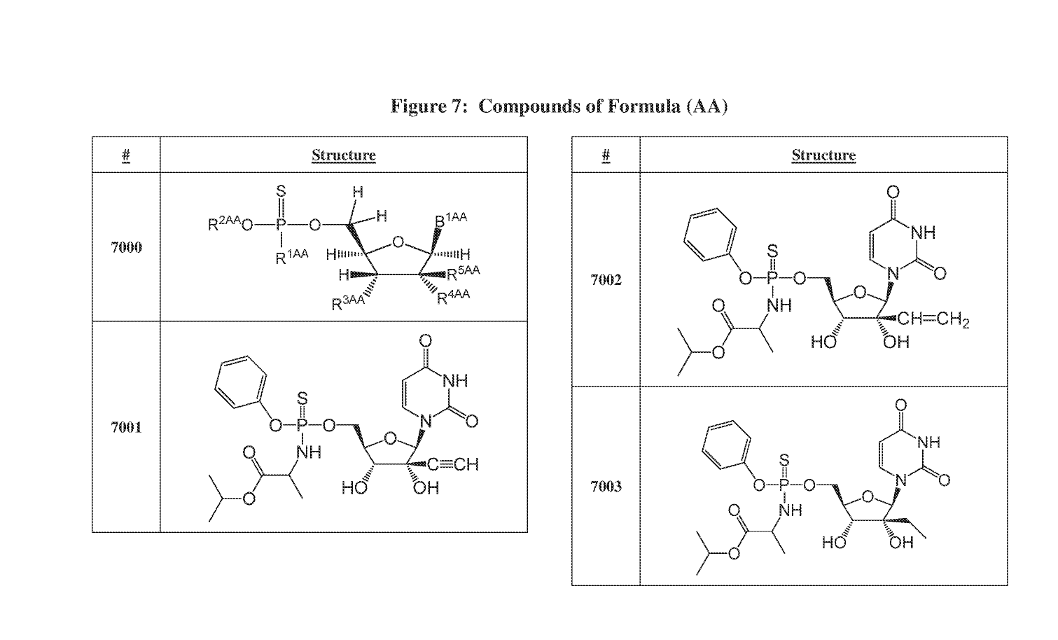

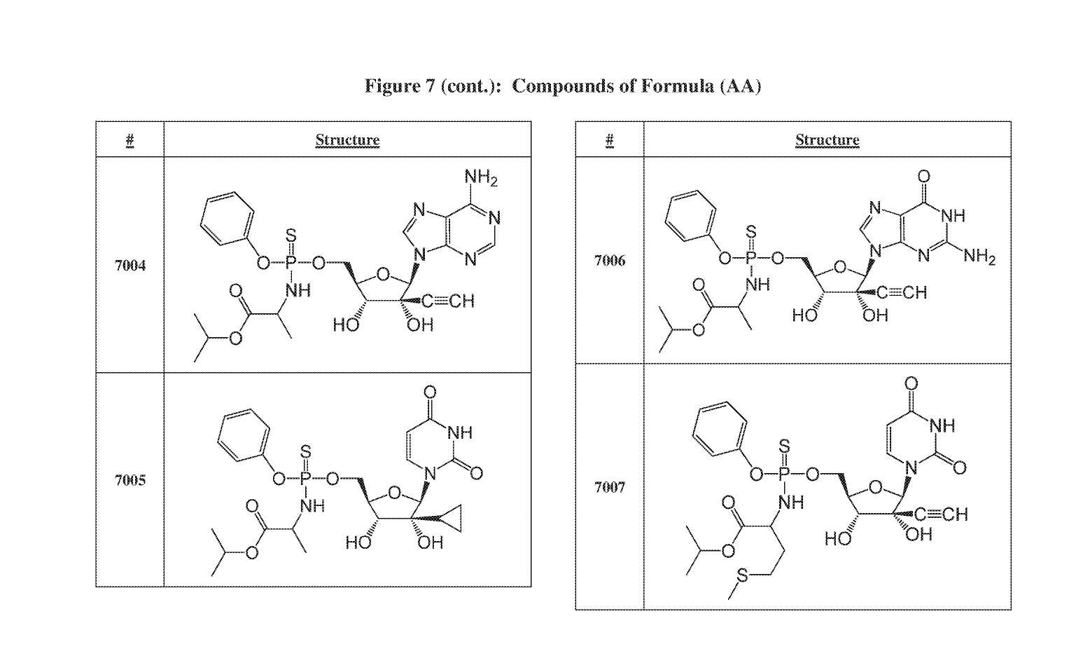

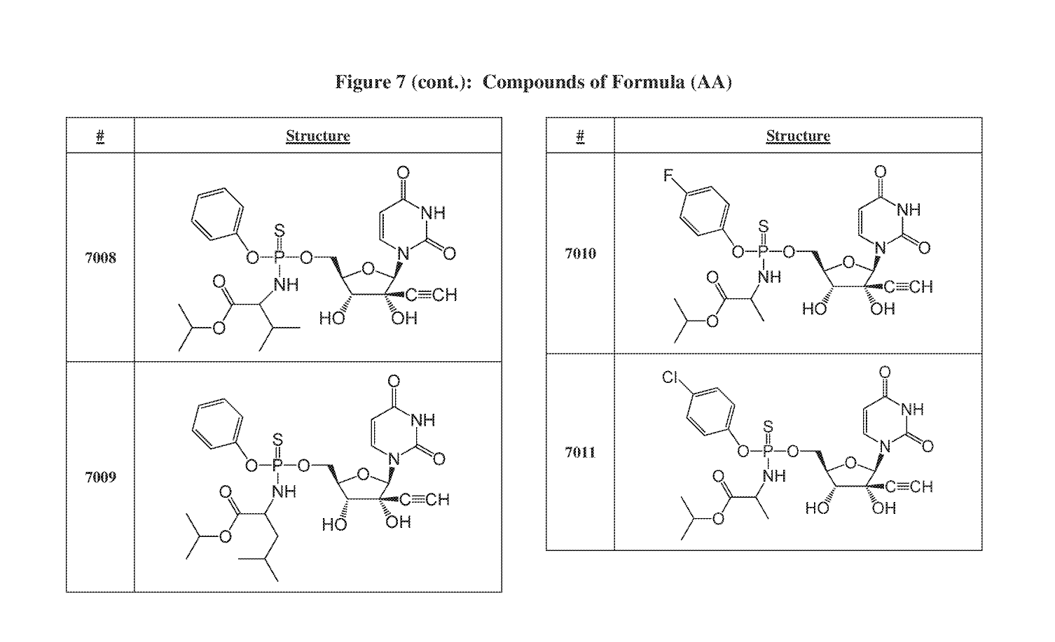

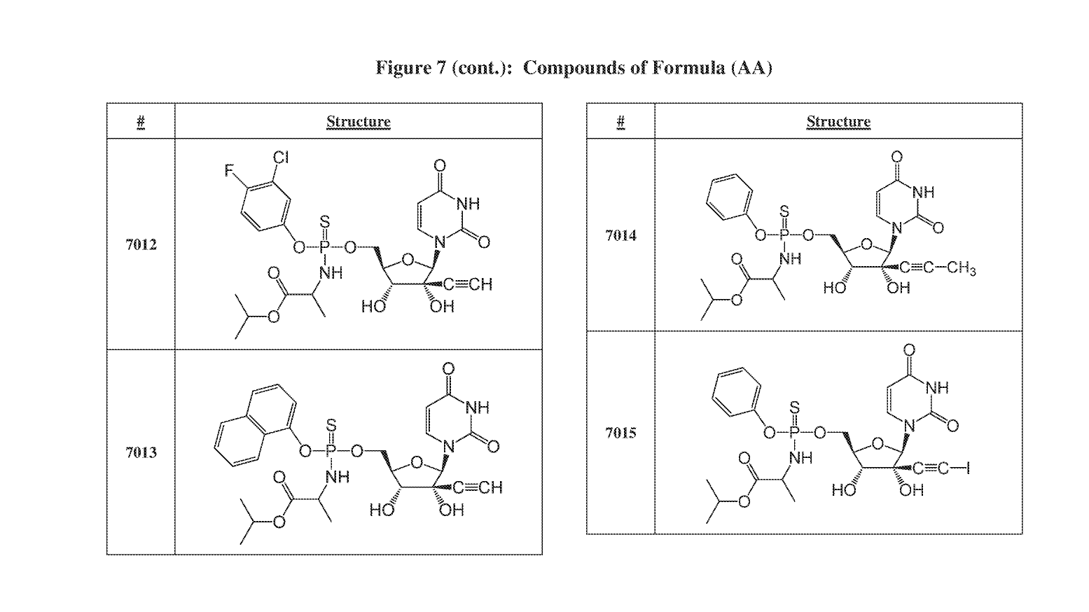

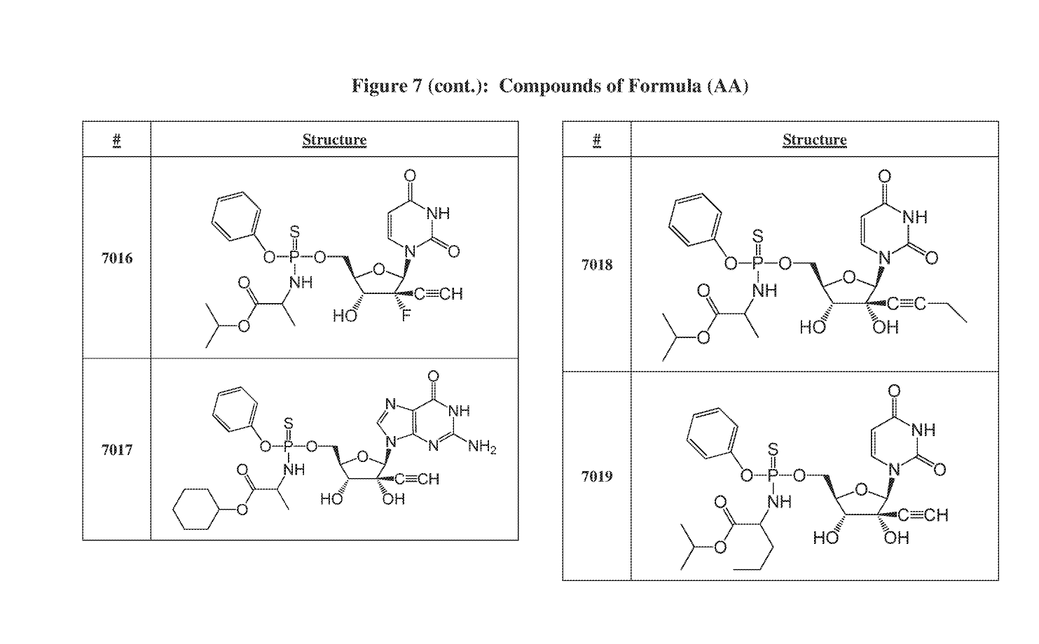

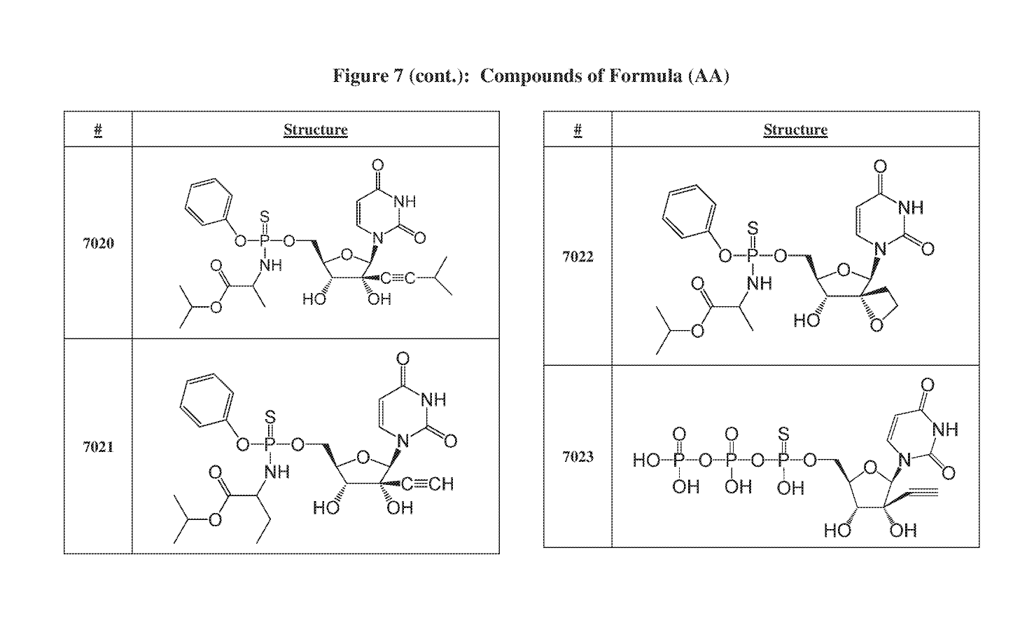

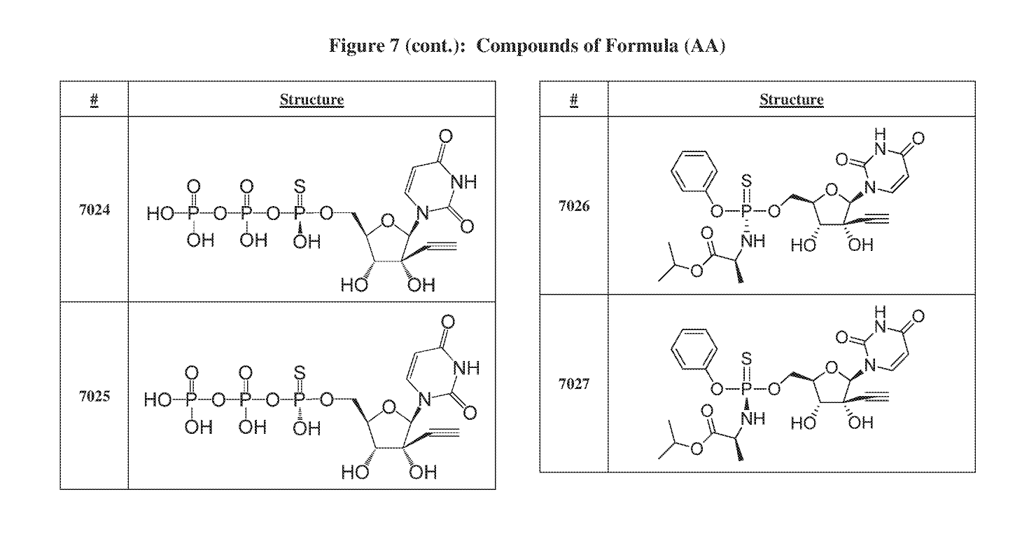

R^{AA3} can be selected from hydrogen, halogen, —OR^{AA9} and —OC(═O)R^{AA10}, R^{AA4} can be selected from halogen, —OR^{AA11} and —OC(═O)R^{AA12}; or R^{AA3} and R^{AA4} can be both an oxygen atom which are linked together by a carbonyl group; R^{AA5} can be selected from an optionally substituted C₂₋₆ alkyl, an optionally substituted C₂₋₆ alkenyl, an optionally substituted C₂₋₆ alkenyl and an optionally substituted C₃₋₆ cycloalkyl; or R^{AA4} and R^{AA5} together can form —(C₁₋₆ alkyl)-O— or —O—(C₁₋₆ alkyl)-; R^{AA9} and R^{AA11} can be independently hydrogen or an optionally substituted C₁₋₆ alkyl; and R^{AA10} and R^{AA12} can be independently an optionally substituted C₁₋₆ alkyl or an optionally substituted C₃₋₆ cycloalkyl. A non-limiting list of examples of compounds of Formula (AA) includes the compounds numbered 7000-7027 in FIG. 7.

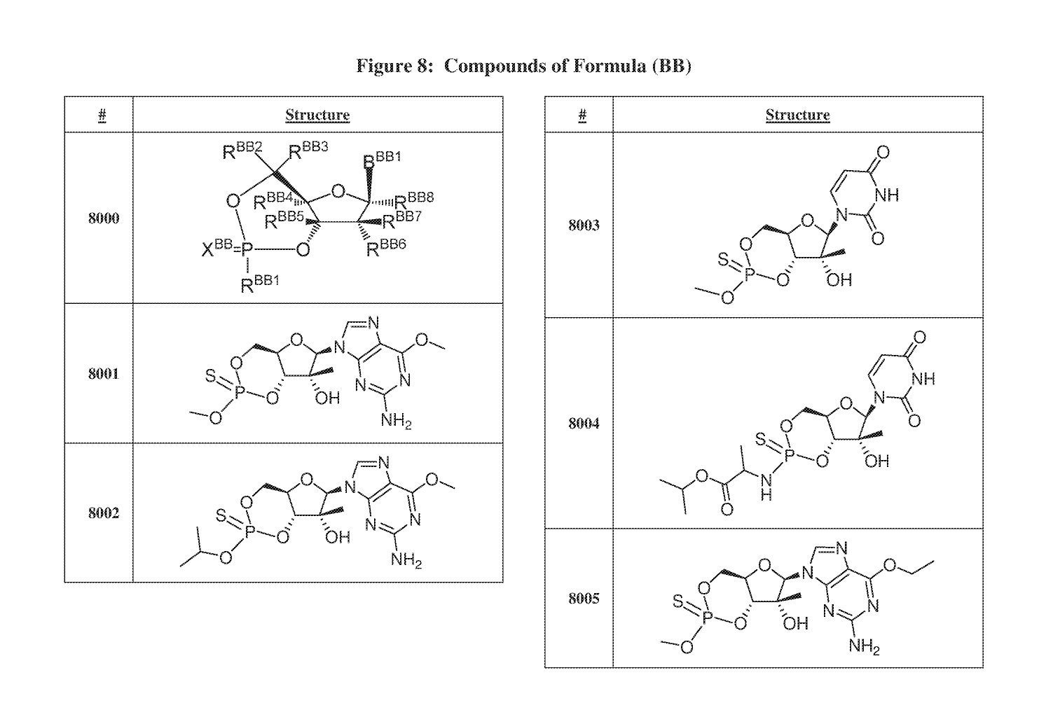

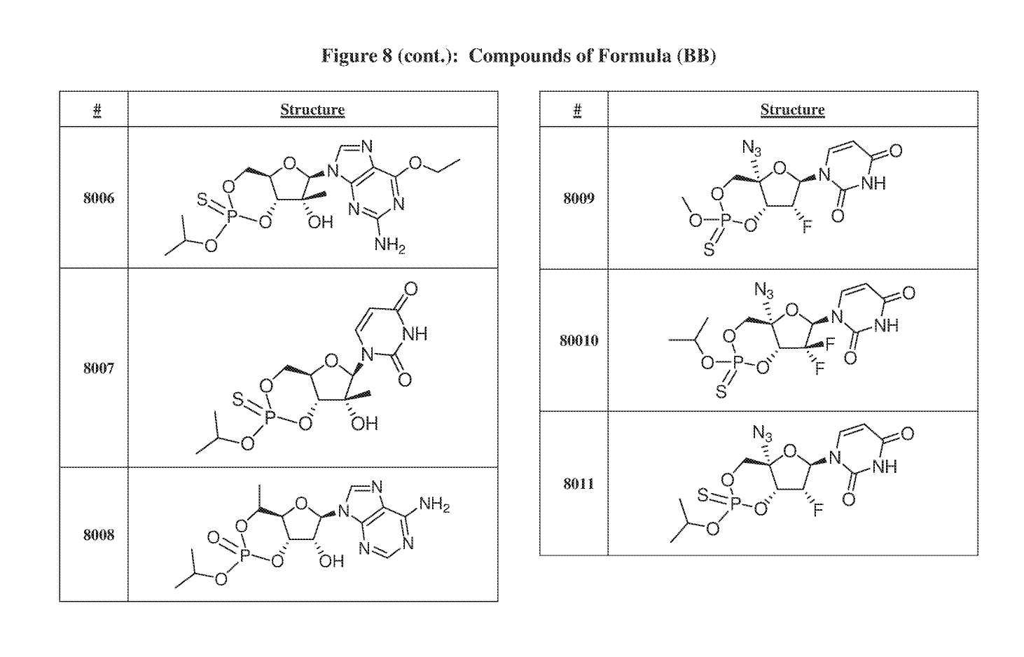

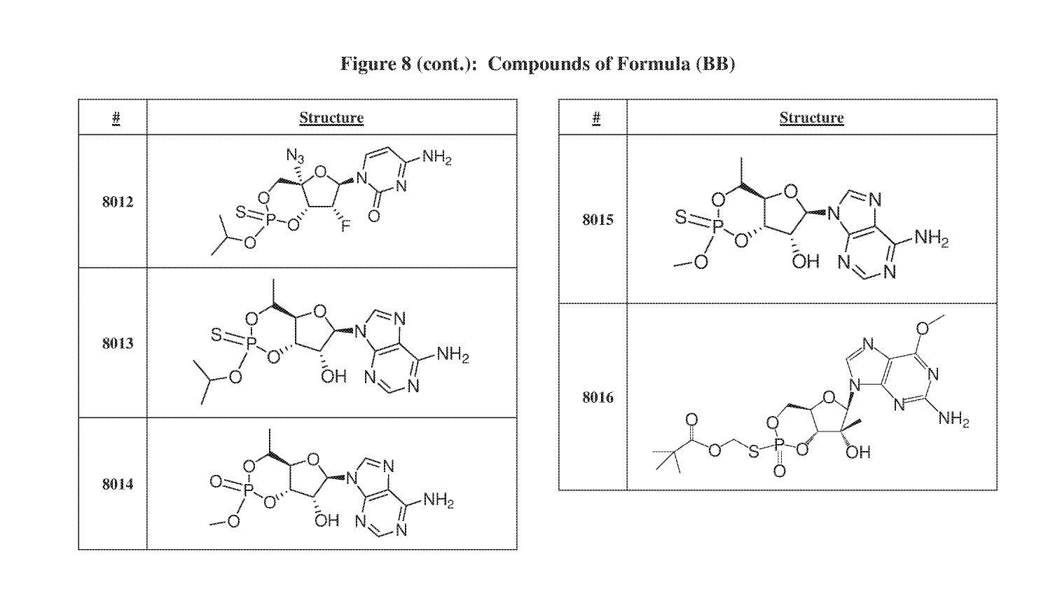

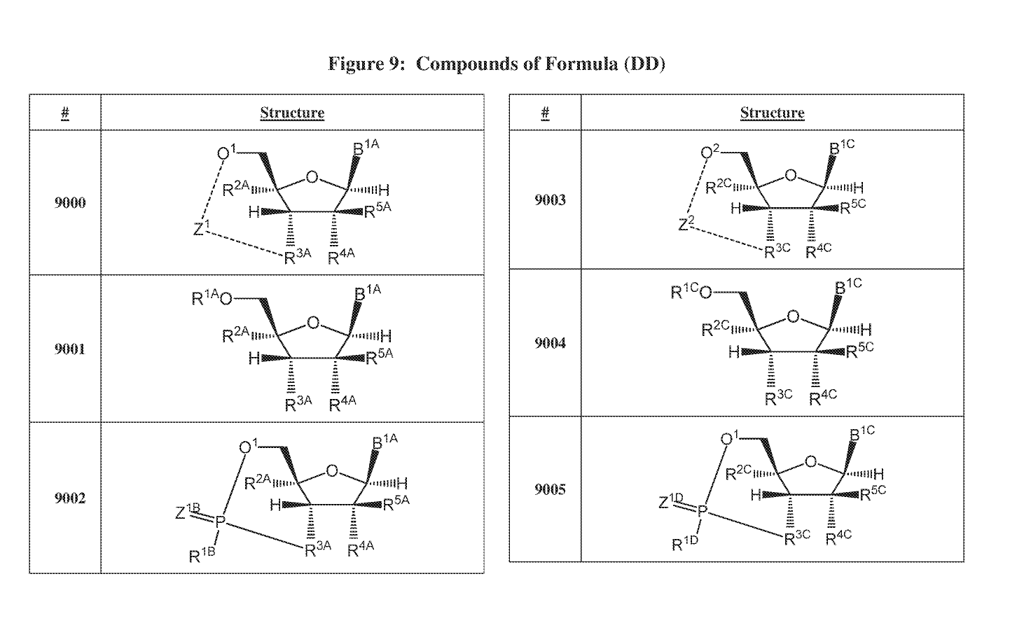

In some embodiments, a compound of Formulae (I) and/or (II), or a pharmaceutically acceptable salt of any of the foregoing, or a pharmaceutical composition that includes a compound of Formulae (I) and/or (II), or a pharmaceutically acceptable salt of any of the foregoing, can be used in combination with a compound of Formula (BB), or a pharmaceutically acceptable salt thereof, or a pharmaceutical composition that includes a compound of Formula (BB), or a pharmaceutically acceptable salt thereof (see, U.S. Publication No. 2012/0165286, published Jun. 28, 2012, the contents of which are incorporated by reference in their entireties):

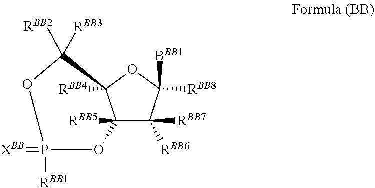

wherein B^{BB1} can be an optionally substituted heterocyclic base or an optionally substituted heterocyclic base with a protected amino group; X^{BB} can be O (oxygen) or S (sulfur); RB^{BB1} can be selected from —Z^{BB}—R^{BB9} an optionally substituted N-linked amino acid and an optionally substituted N-linked amino acid ester derivative; Z^{BB} can be selected from O (oxygen), S (sulfur) and N(R^{BB10});and R^{BB3} can be independently selected from hydrogen, an optionally substituted C₁₋₆ alkyl, an optionally substituted C₂₋₆ alkenyl, an optionally substituted C₂₋₆ alkynyl, an optionally substituted C₁₋₆ haloalkyl and an optionally substituted aryl (C₁₋₆ alkyl); or R^{BB2} and R^{BB3} can be taken together to form a group selected from an optionally substituted C₃₋₆ cycloalkyl, an optionally substituted C₃₋₆ cycloalkenyl, an optionally substituted C₃₋₆ aryl and an optionally substituted C₃₋₆ heteroaryl; R^{BB4} can be selected from hydrogen, halogen, azido, cyano, an optionally substituted C₁₋₆ alkyl, an optionally substituted C₂₋₆ alkenyl, an optionally substituted C₂₋₆ alkynyl and an optionally substituted allenyl; R^{BB5} can be hydrogen or an optionally substituted C₁₋₆ alkyl; R^{BB6} can be selected from hydrogen, halogen, azido, amino, cyano, an optionally substituted C₁₋₆ alkyl, —OR^{BB11} and —OC(═O)R^{BB12}; R^{BB7} can be selected from hydrogen, halogen, azido, cyano, an optionally substituted C₁₋₆ alkyl, —OR^{BB13} and —OC(═O)R^{BB14}; R^{BB8} can be selected from hydrogen, halogen, azido, cyano, an optionally substituted C₁₋₆ alkyl, —OR^{BB15} and —OC(═O)R^{BB16}; R^{BB9} can be selected from an optionally substituted alkyl, an optionally substituted alkenyl, an optionally substituted alkynyl, an optionally substituted cycloalkyl, an optionally substituted cycloalkenyl, an optionally substituted aryl, an optionally substituted heteroaryl, an optionally substituted heterocyclyl, an optionally substituted aryl (C₁₋₆ alkyl), an optionally substituted heteroaryl (C₁₋₆ alkyl) and an optionally substituted heterocyclyl (C₁₋₆ alkyl); R^{BB10} can be selected from hydrogen, an optionally substituted alkyl, an optionally substituted alkenyl, an optionally substituted alkynyl, an optionally substituted cycloalkyl, an optionally substituted cycloalkenyl, an optionally substituted aryl, an optionally substituted heteroaryl, an optionally substituted heterocyclyl, an optionally substituted aryl (C₁₋₆ alkyl), an optionally substituted heteroaryl (C₁₋₆ alkyl) and an optionally substituted heterocyclyl (C₁₋₆ alkyl); R^{BB11}, R^{BB13} and R^{BB15} can be independently hydrogen or an optionally substituted C₁₋₆ alkyl; and R^{BB12}, R^{BB14} and R^{BB16} can be independently an optionally substituted C₁₋₆ alkyl or an optionally substituted C₃₋₆ cycloalkyl. In some embodiments, at least one of R^{BB2} and R^{BB2} is not hydrogen. A non-limiting list of example compounds of Formula (BB) includes the compound numbered 8000-8016 in FIG. 8.

In some embodiments, a compound of Formulae (I) and/or (II), or a pharmaceutically acceptable salt of any of the foregoing, or a pharmaceutical composition that includes a compound of Formulae (I) and/or (II), or a pharmaceutically acceptable salt of any of the foregoing, can be used in combination with a compound of Formula (CC), or a pharmaceutically acceptable salt thereof, or a pharmaceutical composition that includes a compound of Formula (CC), or a pharmaceutically acceptable salt thereof (see, U.S. Publication No. 2012/0071434, published Mar. 22, 2012, the contents of which are incorporated by reference in its entirety):

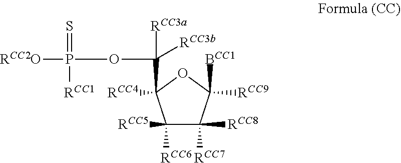

wherein B^{CC1} can be an optionally substituted heterocyclic base or an optionally substituted heterocyclic base with a protected amino group; R^{CC1} can be selected from O⁻, OH, an optionally substituted N-linked amino acid and an optionally substituted N-linked amino acid ester derivative; R^{CC2} can be selected from an optionally substituted aryl, an optionally substituted heteroaryl, an optionally substituted heterocyclyl and

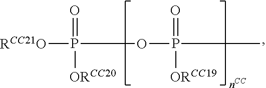

wherein R^{CC19}, C^{CC20} and R^{CC21} can be independently absent or hydrogen and n^{CC} can be 0 or 1; provided that when R^{CC1} is O⁻ or OH, then R^{CC2} is

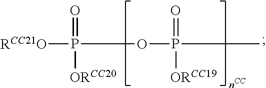

R^{CC3a} and R^{CC3b} can be independently selected from hydrogen, deuterium, an optionally substituted C₁₋₆ alkyl, an optionally substituted C₂₋₆ alkenyl, an optionally substituted C₂₋₆ alkynyl, an optionally substituted C₁₋₆ haloalkyl and aryl (C₁₋₆ alkyl); or R^{CC3a} and R^{CC3b} can be taken together to form an optionally substituted C₃₋₆ cycloalkyl; R^{CC4} can be selected from hydrogen, azido, an optionally substituted C₁₋₆ alkyl, an optionally substituted C₂₋₆ alkenyl and an optionally substituted C₂₋₆ alkynyl; R^{CC5} can be selected from hydrogen, halogen, azido, cyano, an optionally substituted C₁₋₆ alkyl, —OR^{CC10} and —OC(═O)R^{CC11}; R^{CC6} can be selected from hydrogen, halogen, azido, cyano, an optionally substituted C₁₋₆ alkyl, —OR^{CC12} and —OC(═O)R^{CC13}; R^{CC7} can be selected from hydrogen, halogen, azido, cyano, an optionally substituted C₁₋₆ alkyl, —OR^{CC14} and —OC(═O)R^{CC15}; or R^{CC6} and R^{CC7} can be both oxygen atoms and linked together by a carbonyl group; R^{CC8} can be selected from hydrogen, halogen, azido, cyano, an optionally substituted C₁₋₆ alkyl, —OR^{CC16} and —OC(═O)R_{CC17}; R^{CC 9} can be selected from hydrogen, azido, cyano, an optionally substituted C₁₋₆ alkyl and —OR^{CC18}; R^{CC10}, R^{CC12}, R^{CC14}, R^{CC16} and R^{CC18} can be independently selected from hydrogen and an optionally substituted C₁₋₆ alkyl; and R^{CC11}, R^{CC13}, R^{CC15} and R^{CC17} can be independently selected from an optionally substituted C₁₋₆ alkyl and an optionally substituted C₃₋₆ cycloalkyl. In some embodiments, when R^{CC3a}, R^{CC3b}, R^{CC4}, R^{CC5}, R^{CC7}, R^{CC8} and R^{CC9} are all hydrogen, then R^{CC6} is not azido. In some embodiments, R^{CC2} cannot be

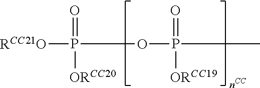

when R^{CC3a} is a hydrogen, R^{CC3b} is hydrogen, R^{CC4} is H, R^{CC5} is OH or H, R^{CC6} is hydrogen, OH, or —OC(═O)CH₃, R^{CC7} is hydrogen, OH, OCH₃ or —OC(═O)CH₃, R^{CC8} is hydrogen, OH or OCH₃, R^{CC9} is H and B^{CC1} is an optionally substituted adenine, an optionally substituted guanine, an optionally substituted uracil or an optionally substituted hypoxanthine. In some embodiments, R^{CC2} cannot be

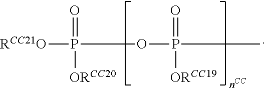

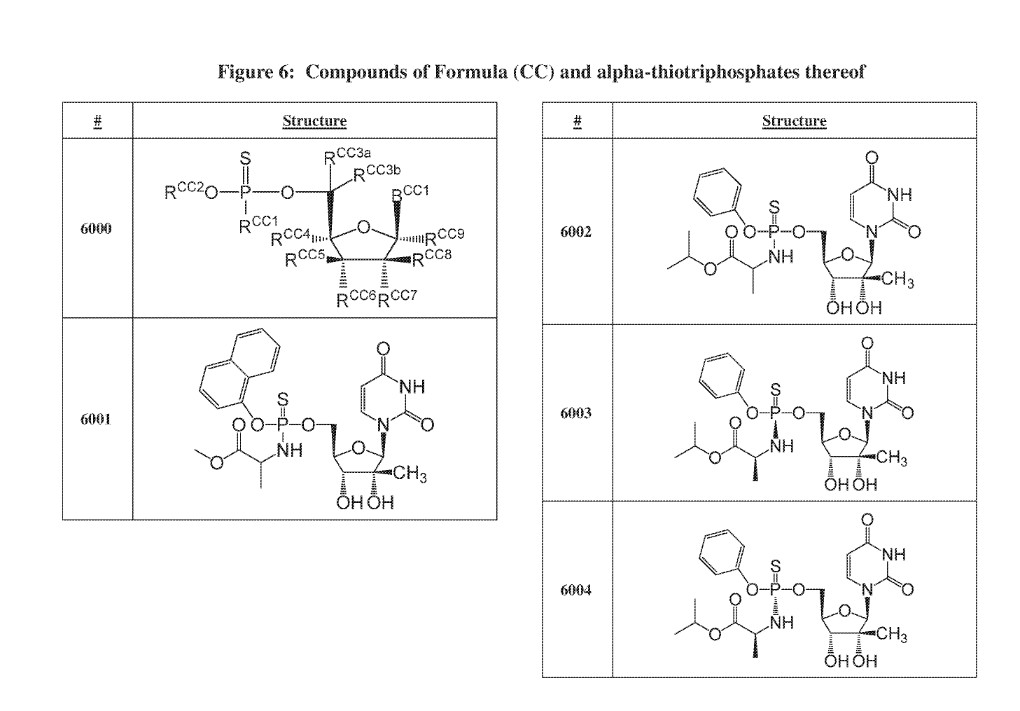

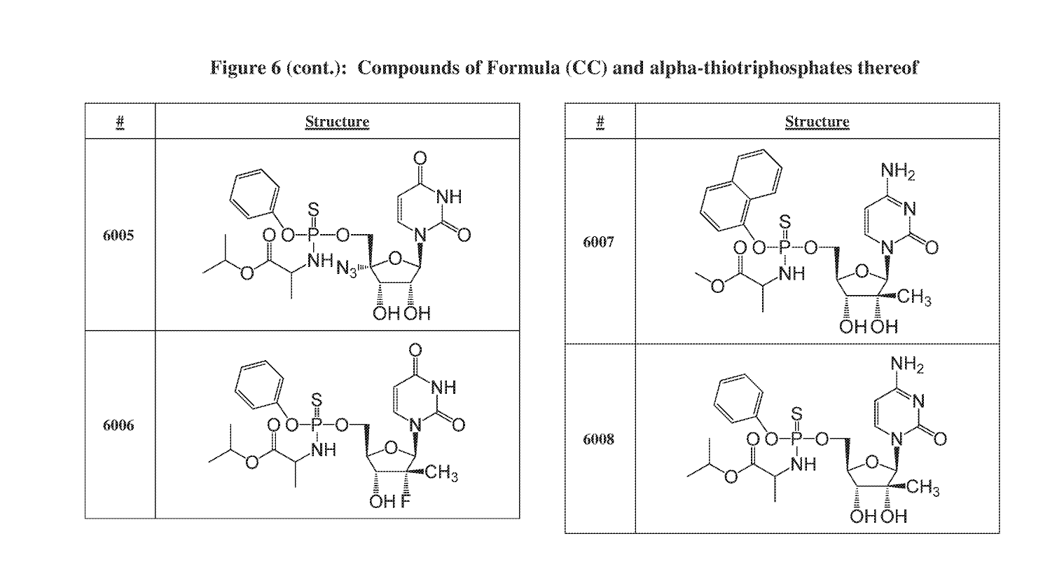

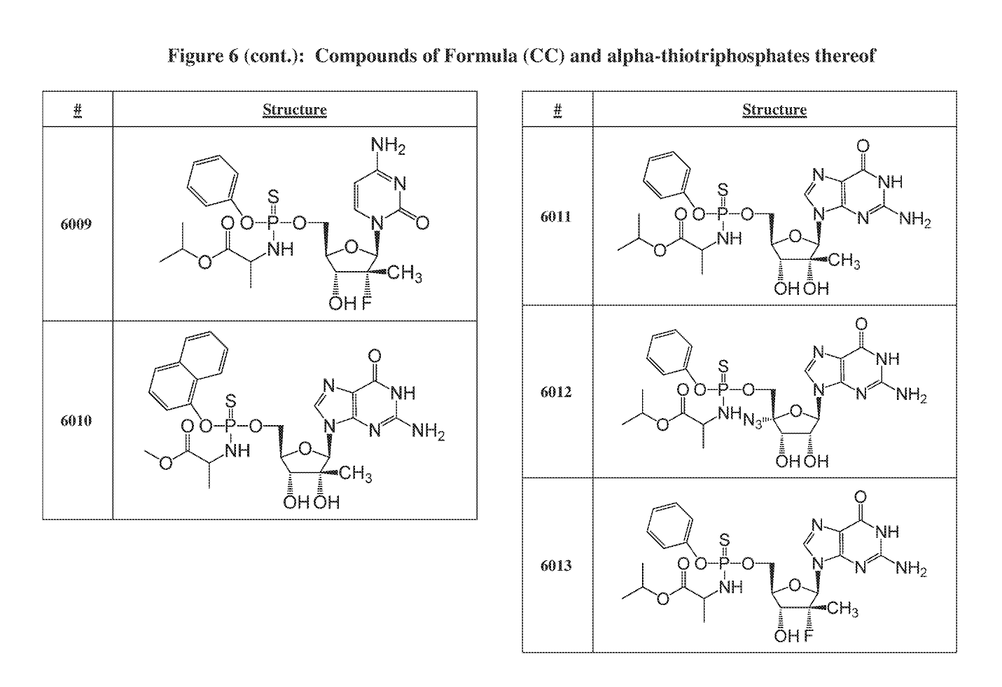

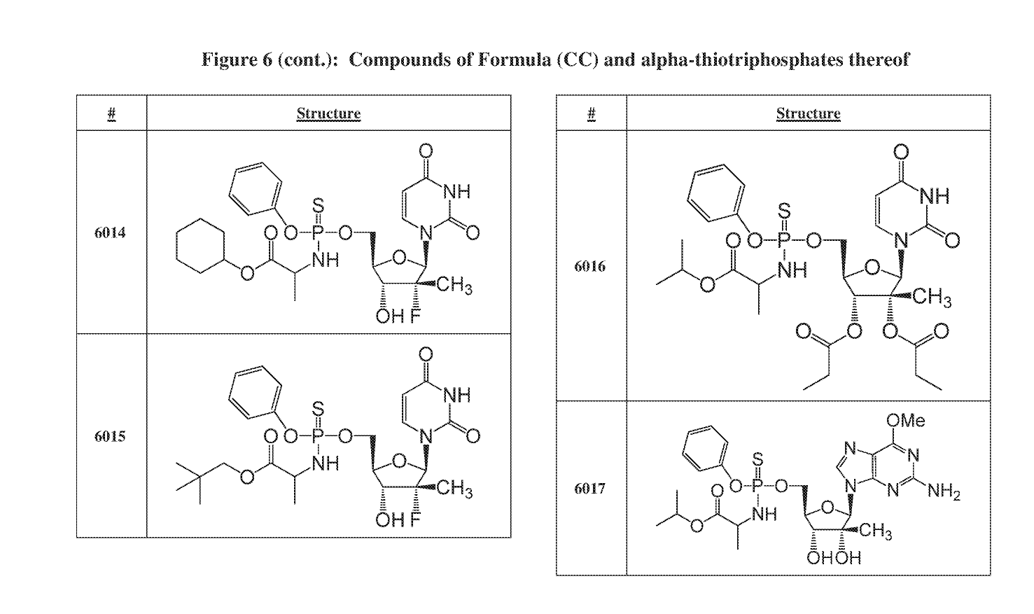

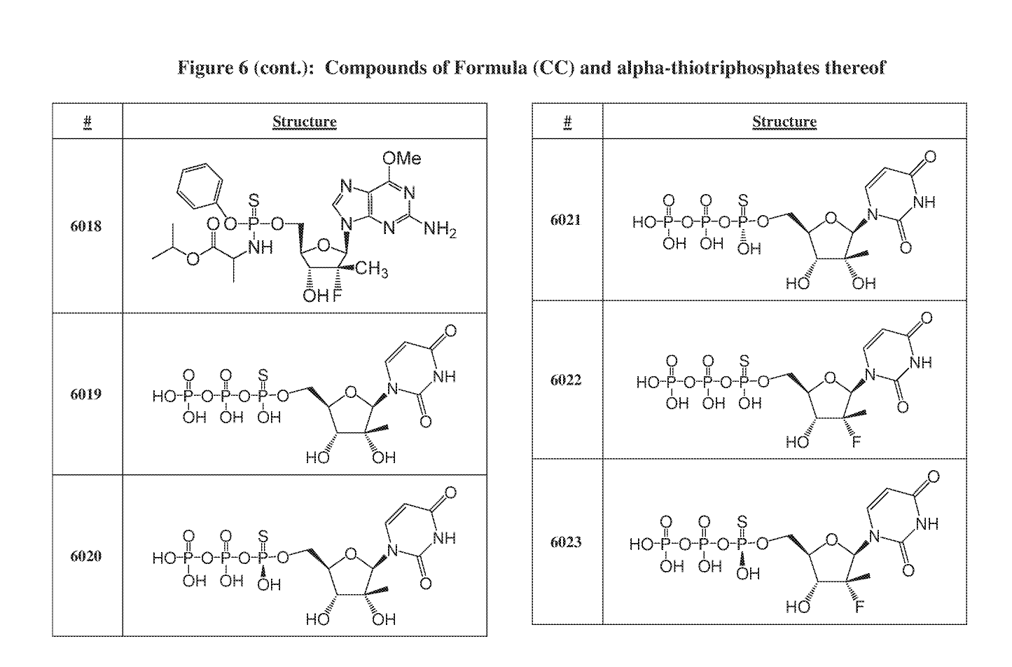

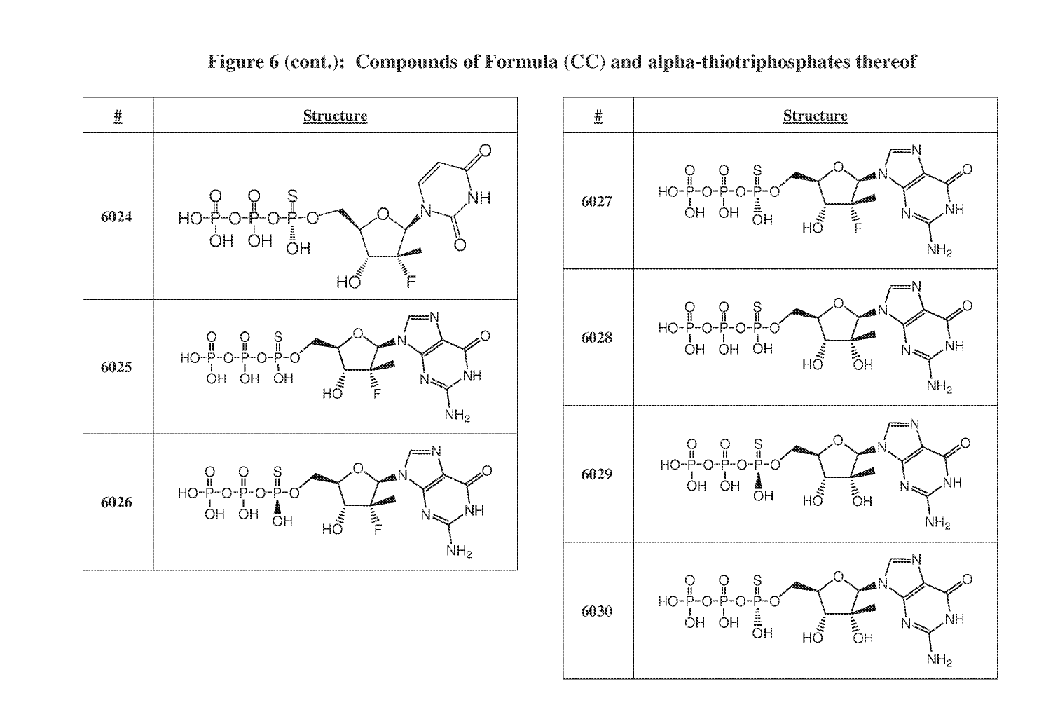

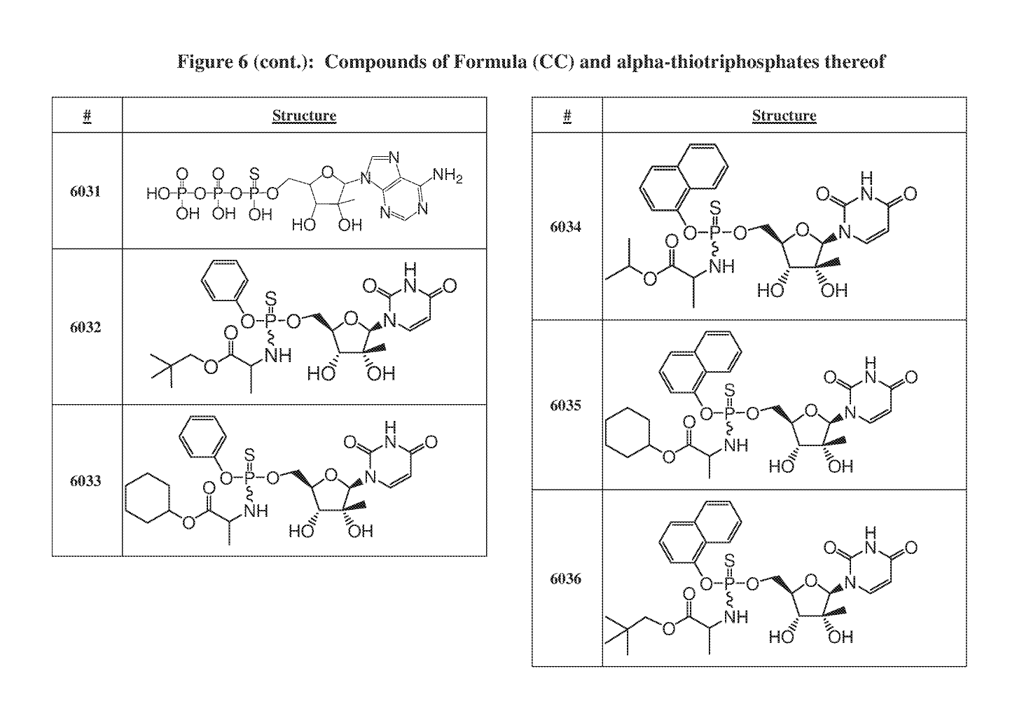

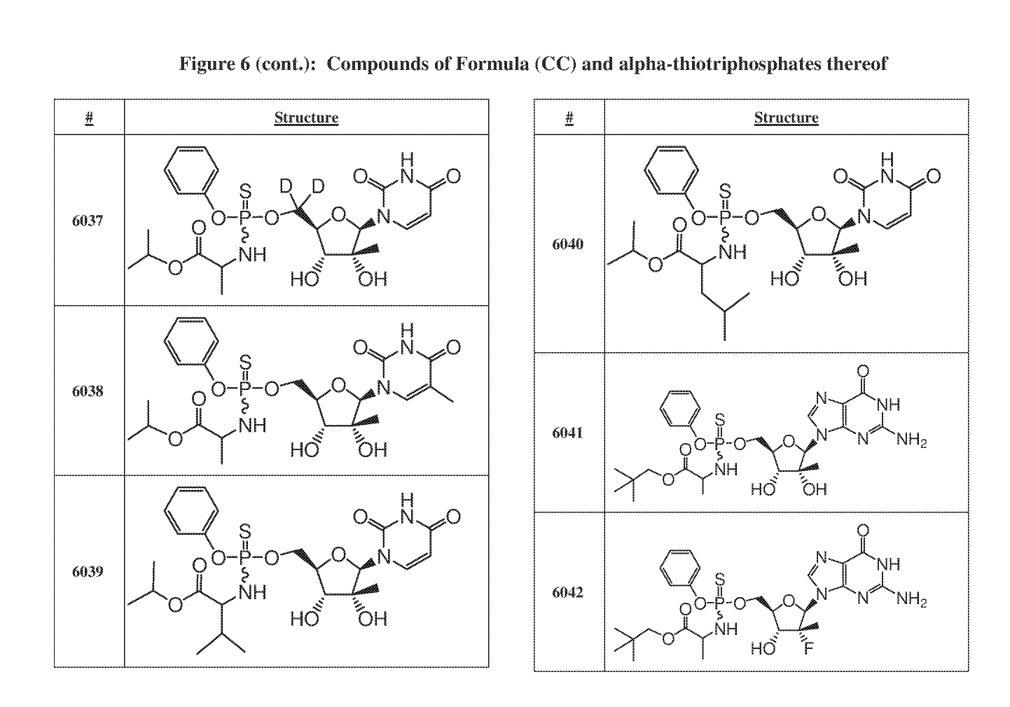

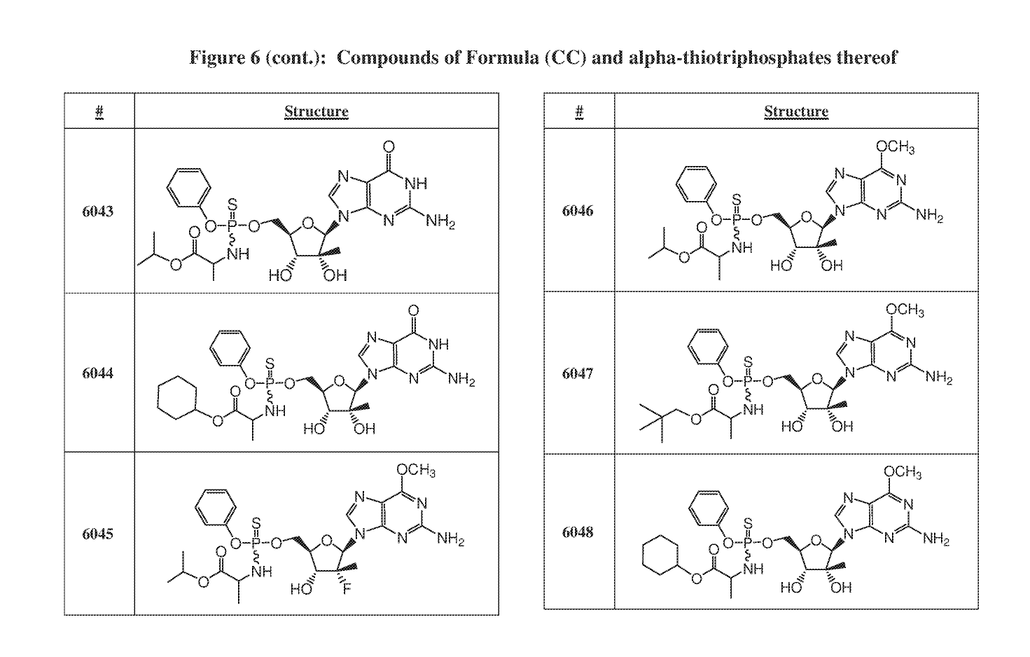

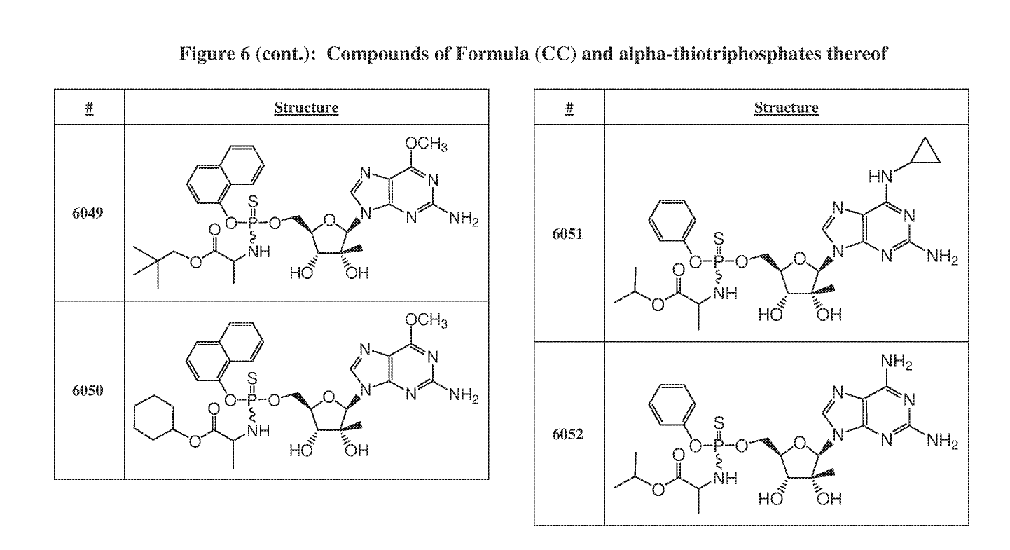

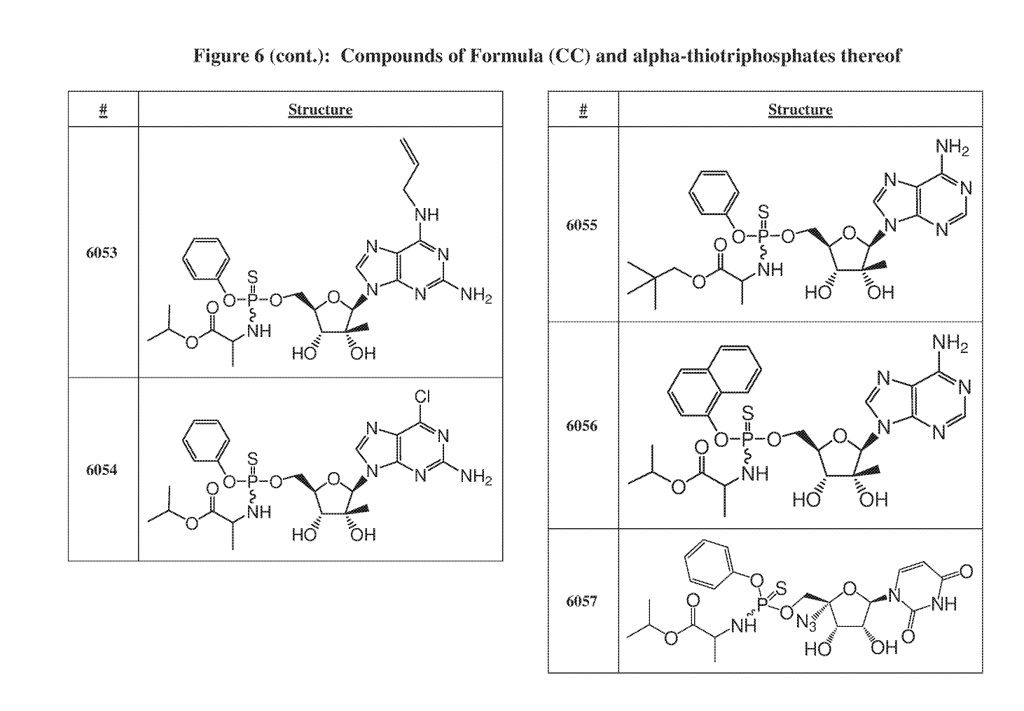

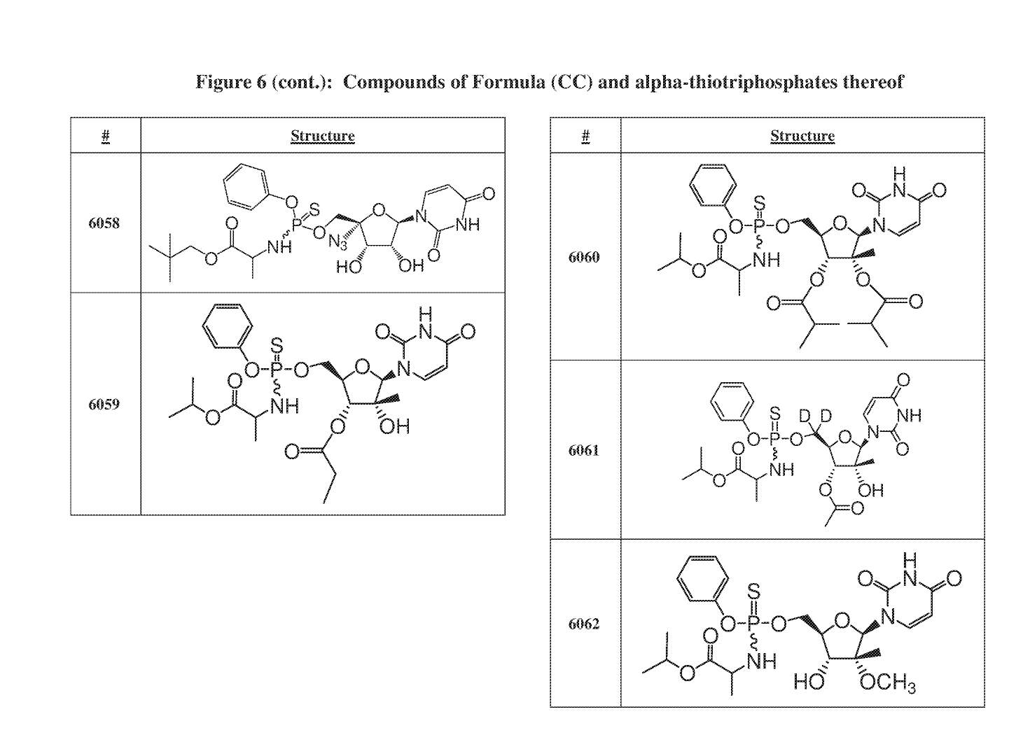

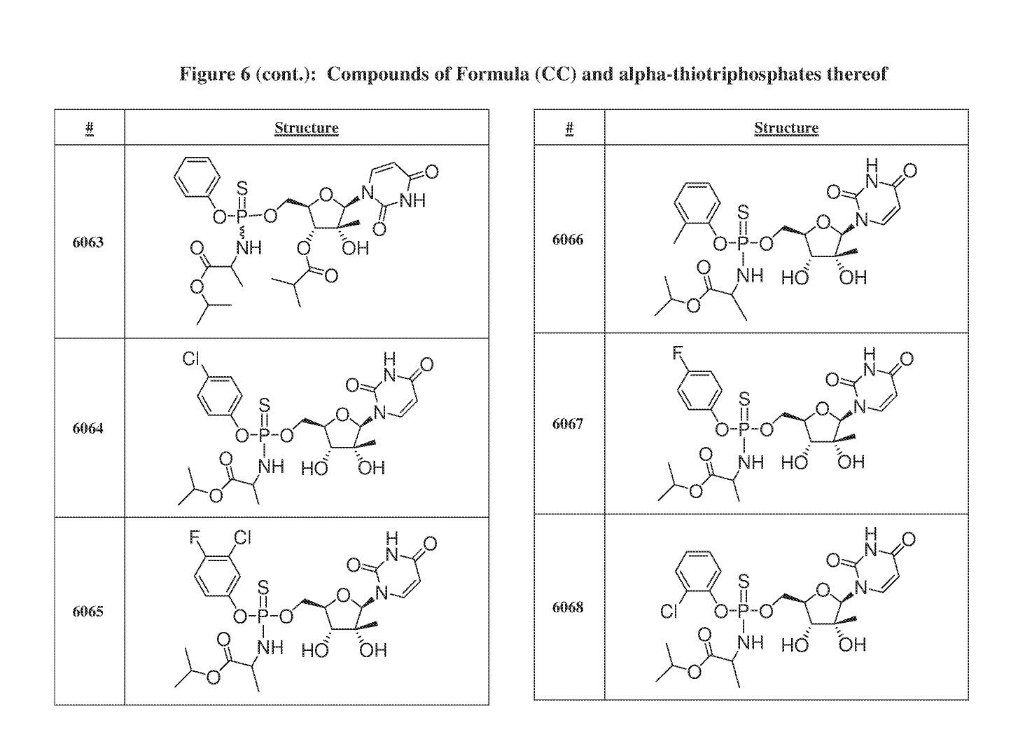

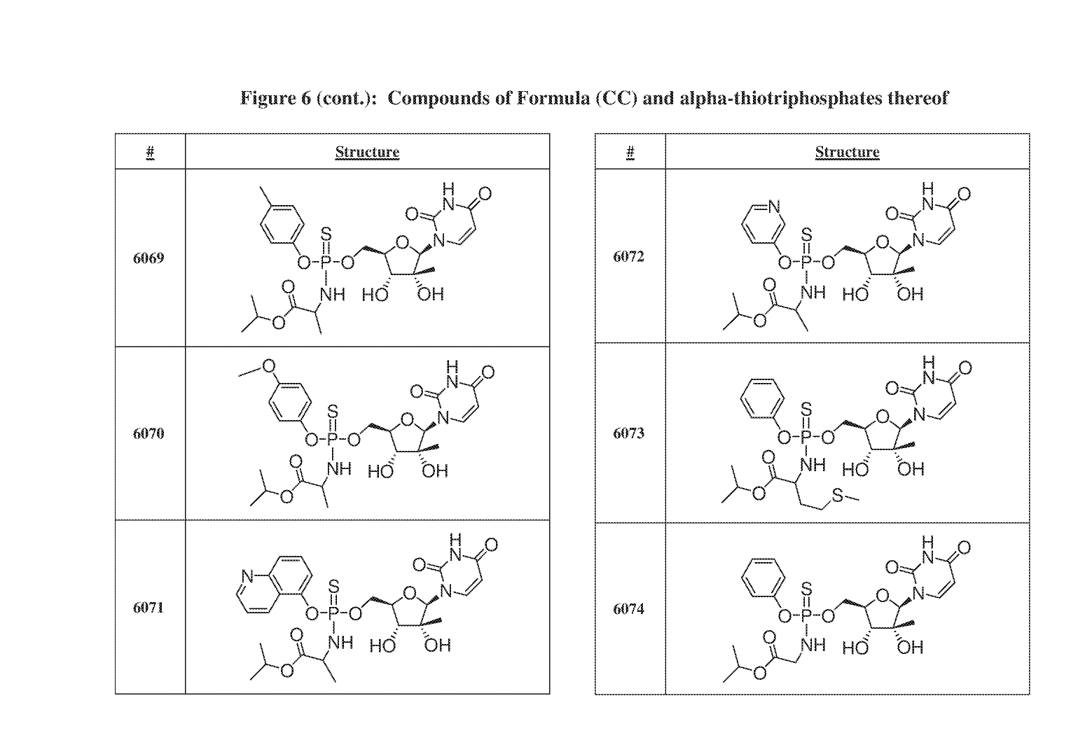

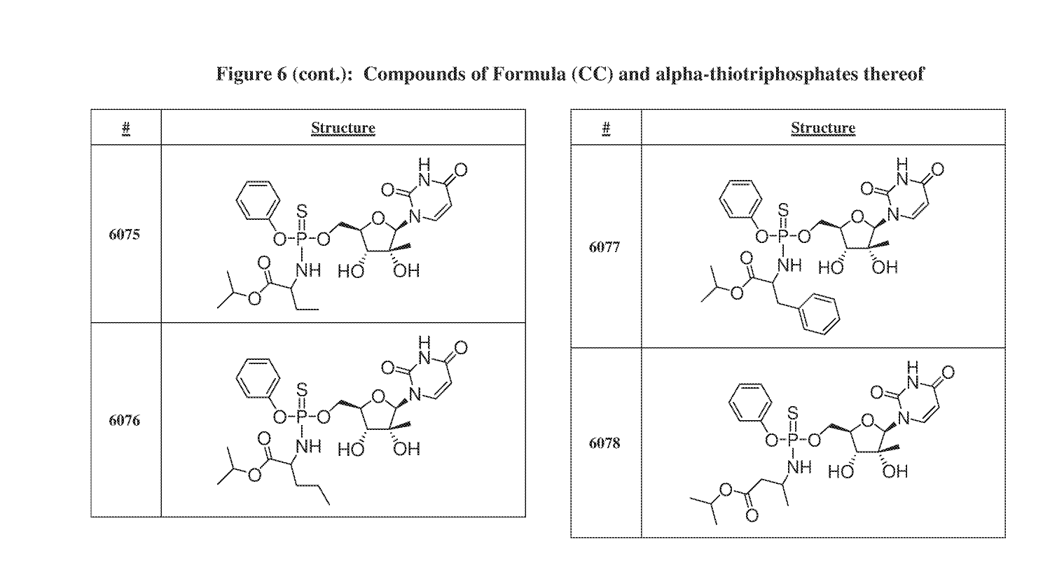

A non-limiting list of examples of compounds of Formula (CC) includes the compounds numbered 6000-6078 in FIG. 6.

In some embodiments, a compound of Formulae (I) and/or (II), or a pharmaceutically acceptable salt of any of the foregoing, or a pharmaceutical composition that includes a compound of Formulae (I) and/or (II), or a pharmaceutically acceptable salt of any of the foregoing, can be used in combination with a compound of Formula (DD), or a pharmaceutically acceptable salt thereof, or a pharmaceutical composition that includes a compound of Formula (DD), or a pharmaceutically acceptable salt thereof (see, U.S. Publication No. 2015/0105341 published Apr. 16, 2015, the contents of which are incorporated by reference in its entirety):

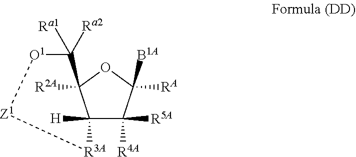

wherein: B^{1A} can be an optionally substituted heterocyclic base or an optionally substituted heterocyclic base with a protected amino group; -------- can be absent or a single bond, provided that both -------- are absent or both -------- are a single bond; when -------- are both absent, then Z¹ can be absent, O¹ can be OR^{1A}, R^{3A} can be selected from H, halo, OH, —OC(═O)R″^{A} and an optionally substituted O-linked amino acid, R^{4A} can be selected from H, OH, halo, N₃, —OC(═O)R″^{B}, an optionally substituted O-linked amino acid and NR″^{B1}R″^{B2}, or R^{3A} and R^{4A} can be both an oxygen atom connected via a carbonyl to form a 5-membered ring; when -------- are each a single bond, then Z¹ can be

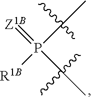

O¹ can be O, R^{3A} can be O, R^{4A} can be selected from H, OH, halo, N₃, —OC(═O)R″^{B}, an optionally substituted O-linked amino acid and NR″^{B1}R″^{B2}; and R^{1B} can be selected from O⁻, OH, an —O-optionally substituted C₁₋₆ alkyl.

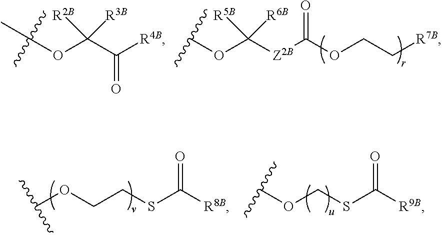

an optionally substituted N-linked amino acid and an optionally substituted N-linked amino acid ester derivative; R^{a1} and R^{a2} can be independently hydrogen or deuterium; R^{A} can be hydrogen, deuterium, an unsubstituted alkyl, an unsubstituted C₂₋₄ alkenyl, an unsubstituted C₂₋₃ alkynyl or cyano; R^{1A} can be selected from hydrogen, an optionally substituted acyl, an optionally substituted O-linked amino acid,

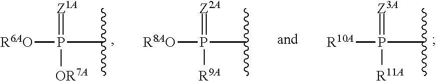

R^{2A} can be hydrogen, halo, an unsubstituted C₁₋₄ alkyl, an unsubstituted C₂₋₄ alkenyl, an unsubstituted C₂₋₄ alkynyl, —CHF₂, —(CH₂)₁₋₆ halogen, —(CH₂)₁₋₆N₃, —(CH₂)₁₋₆NH₂ or —CN; R^{5A} can be selected from H, halo, OH, an optionally substituted C₁₋₆ alkyl, an optionally substituted C₂₋₆ alkenyl and an optionally substituted C₂₋₆ alkynyl; R^{6A}, R^{7A} and R^{8A} can be independently selected from absent, hydrogen, an optionally substituted C₁₋₂₄ alkyl, an optionally substituted C₂₋₂₄ alkenyl, an optionally substituted C₂₋₂₄ alkynyl, an optionally substituted C₃₋₆ cycloalkyl, an optionally substituted C₃₋₆ cycloalkenyl, an optionally substituted aryl, an optionally substituted heteroaryl, an optionally substituted aryl (C₁₋₆ alkyl), an optionally substituted *—(CR^{15A}R^{16A})ₚ—O—C₁₋₂₄ alkyl, an optionally substituted *—(CR^{17A}R^{18A})_{q}—O—C₁₋₂₄ alkenyl,

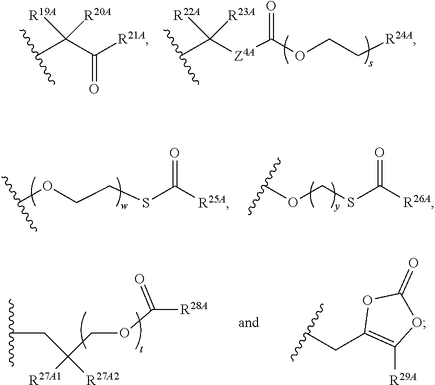

### or R^{6A} can be

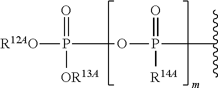

and R^{7A} can be absent or hydrogen; or R^{6A} and R^{7A} can be taken together to form a moiety selected from an optionally substituted

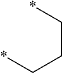 [SMILES: *CCC*]

and an optionally substituted

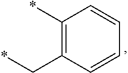 [SMILES: *Cc1ccccc1*]

wherein the oxygens connected to R^{6A} and R^{7A}, the phosphorus and the moiety form a six-membered to ten-membered ring system; R^{9A} can be independently selected from an optionally substituted C₁₋₂₄ alkyl, an optionally substituted C₂₋₂₄ alkenyl, an optionally substituted C₂₋₂₄ alkynyl, an optionally substituted C₃₋₆ cycloalkyl, an optionally substituted C₃₋₆ cycloalkenyl, NR^{30A}R^{31A}, an optionally substituted N-linked amino acid and an optionally substituted N-linked amino acid ester derivative; R^{10A} and R^{11A} can be independently an optionally substituted N-linked amino acid or an optionally substituted N-linked amino acid ester derivative; R^{12A} and R^{13A} can be independently absent or hydrogen; R^{14A} can be O—, OH or methyl; each R^{15A}, each R^{16A}, each R^{17A} and each R^{18A} can be independently hydrogen, an optionally substituted C₁₋₂₄ alkyl or an alkoxy; R^{19A}, R^{20A}, R^{22A}, R^{23A}, R^{2B}, R^{3B}, R^{5B} and R^{6B} can be independently selected from hydrogen, an optionally substituted C₁₋₂₄ alkyl and an optionally substituted aryl; R^{21A} and R^{4B} can be independently selected from hydrogen, an optionally substituted C₁₋₂₄ alkyl, an optionally substituted aryl, an optionally substituted —O—C₁₋₂₄ alkyl, an optionally substituted —O-aryl, an optionally substituted —O-heteroaryl and an optionally substituted —O-monocyclic heterocyclyl; R^{24A} and R^{7B} can be independently selected from of hydrogen, an optionally substituted C₁₋₂₄ alkyl, an optionally substituted aryl, an optionally substituted —O—C₁₋₂₄ alkyl, an optionally substituted —O-aryl, an optionally substituted —O-heteroaryl, an optionally substituted —O-monocyclic heterocyclyl and

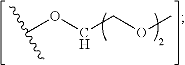

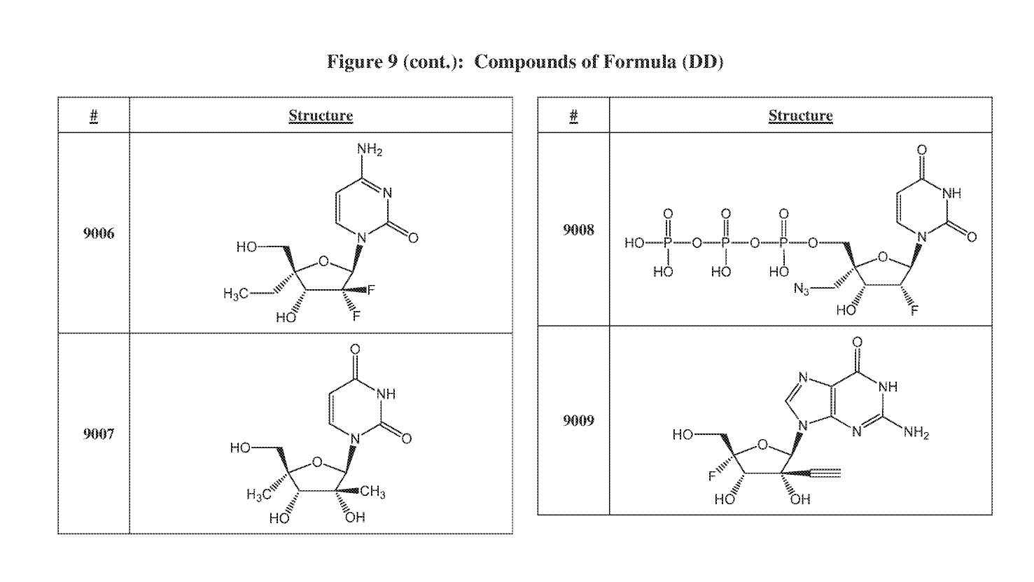

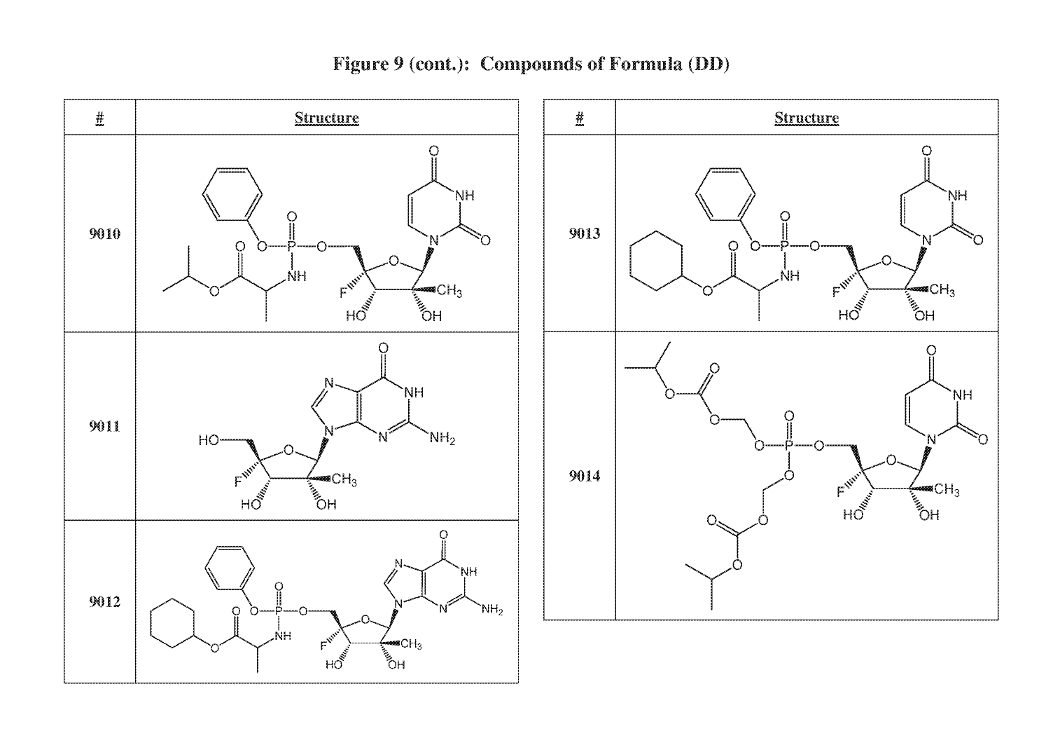

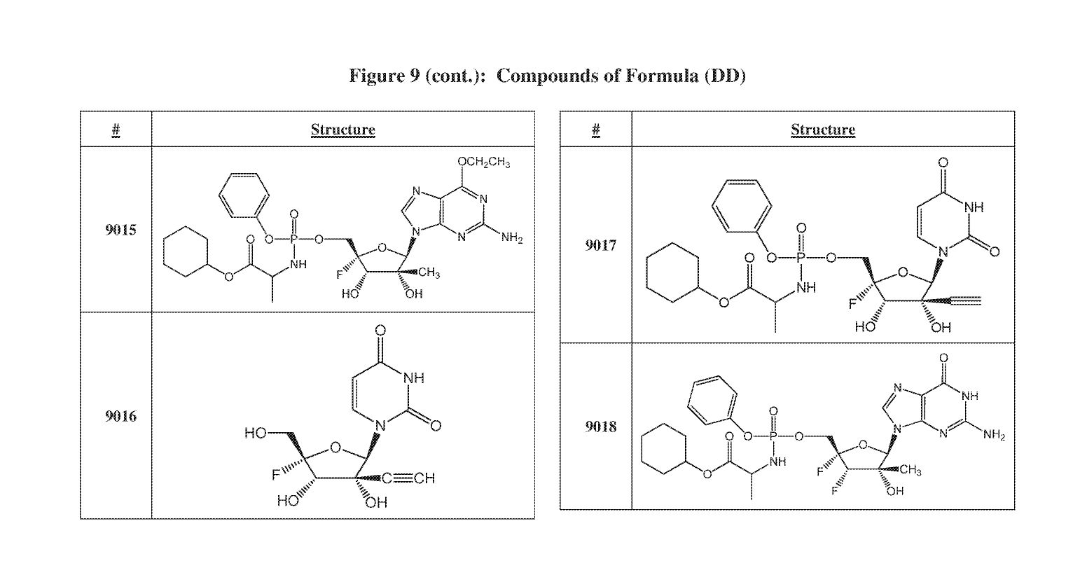

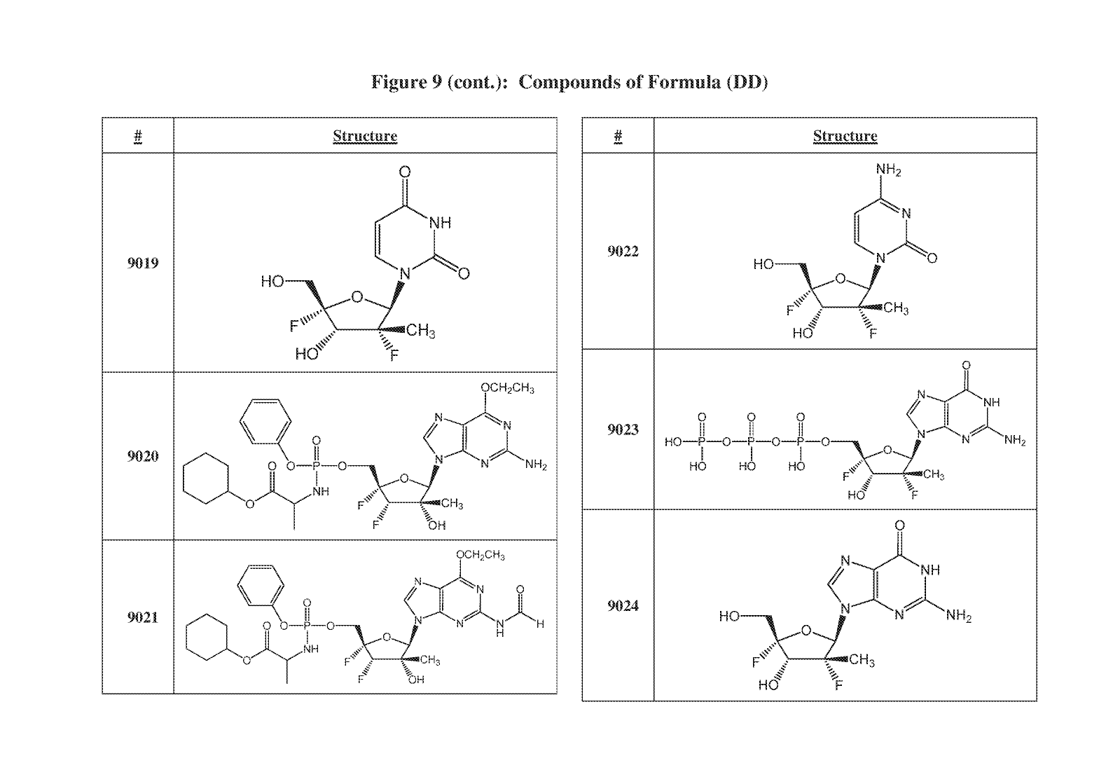

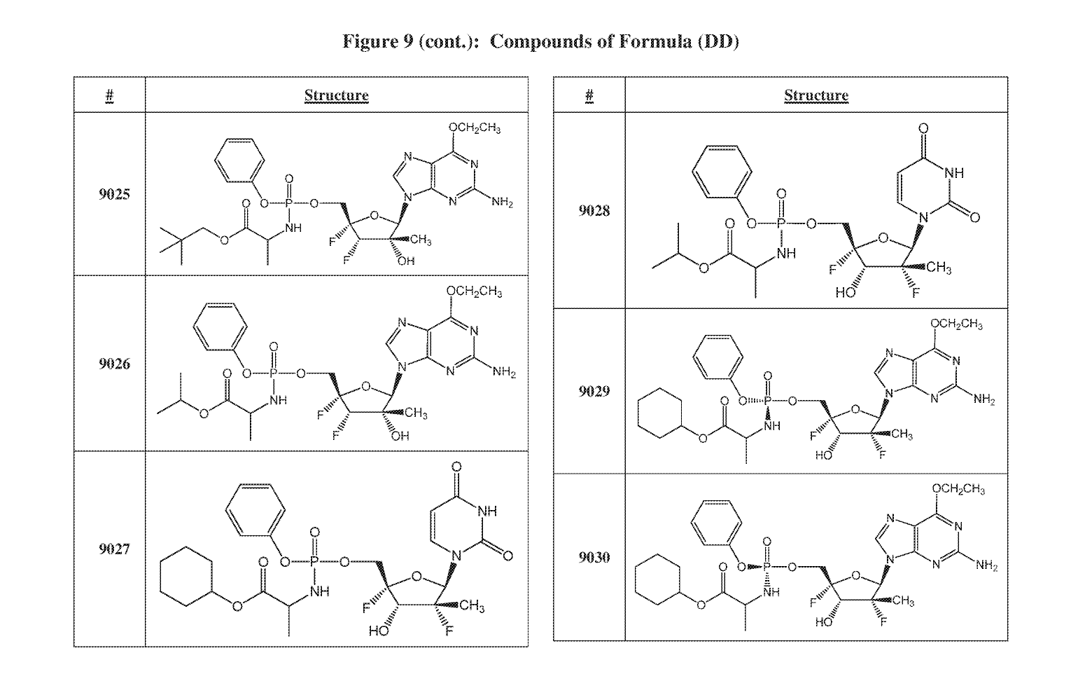

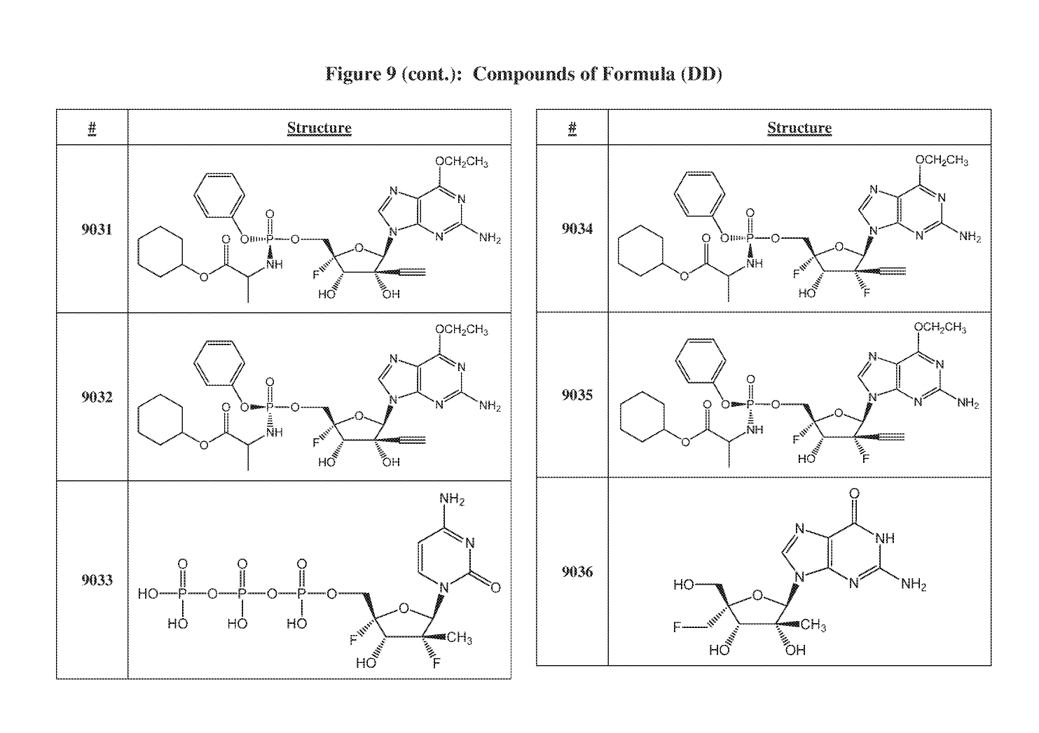

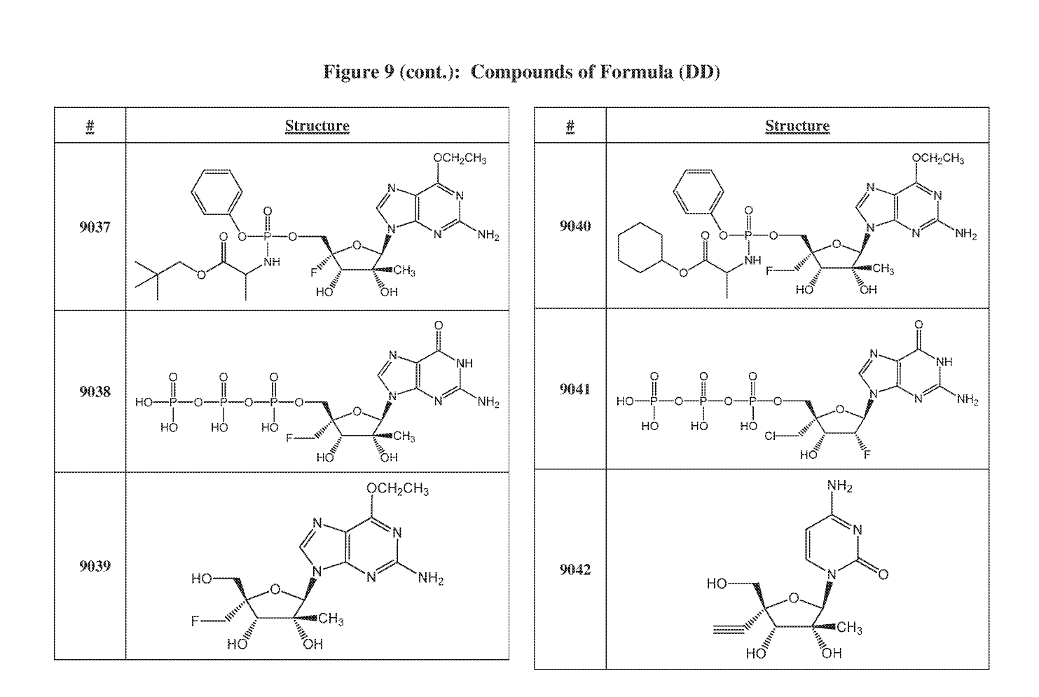

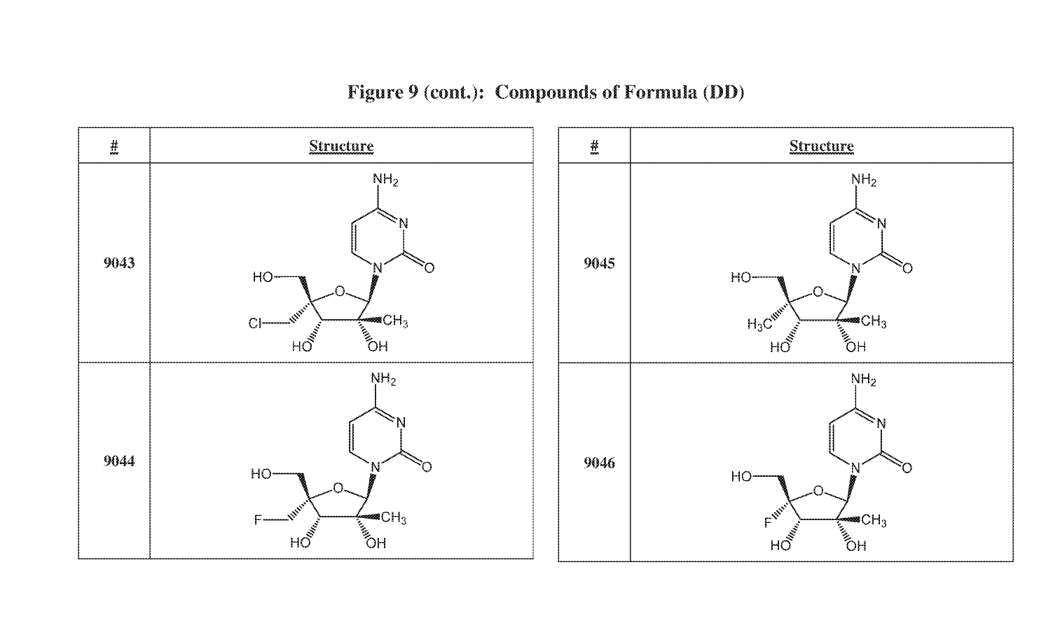

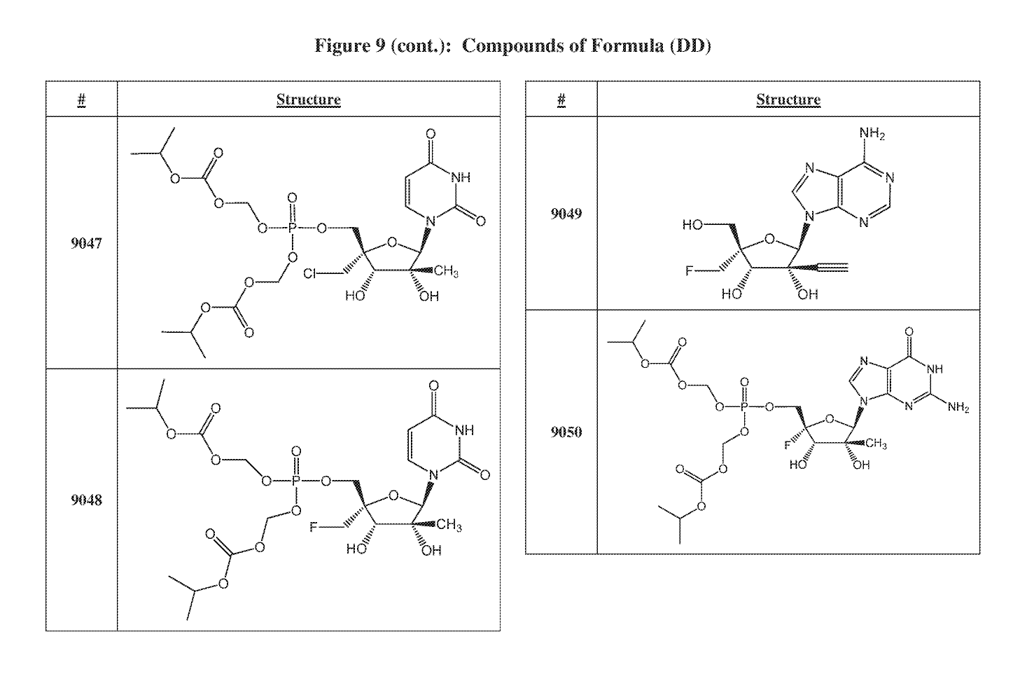

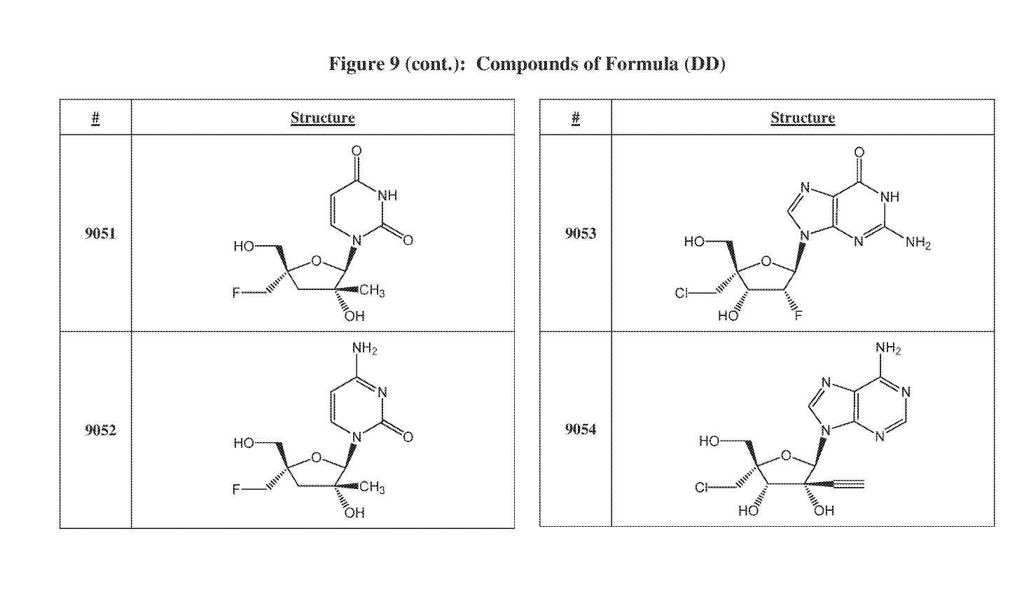

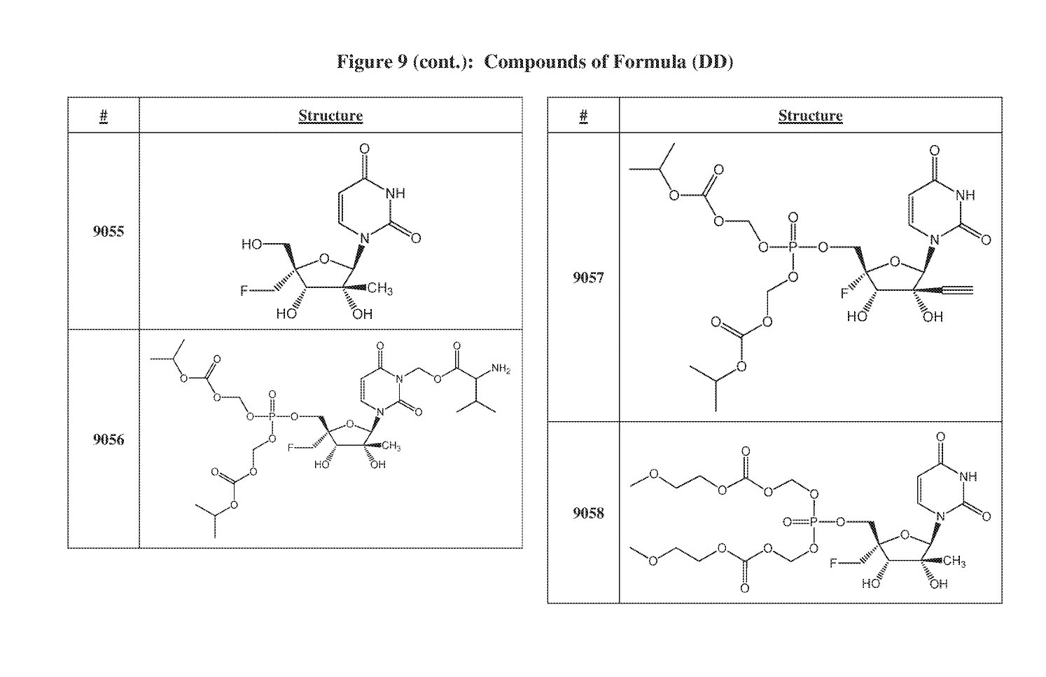

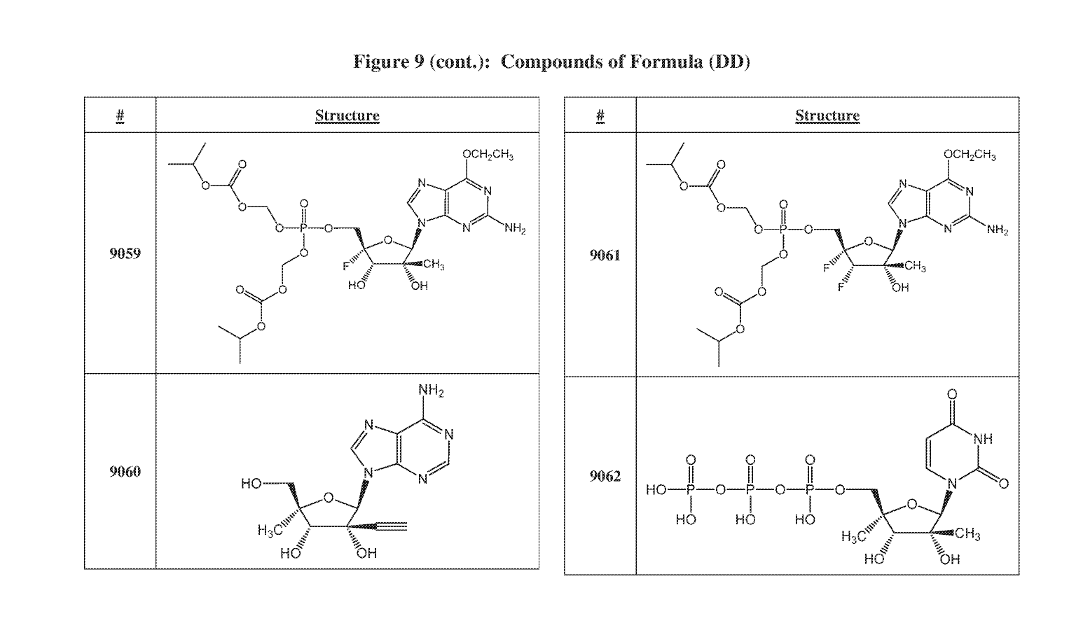

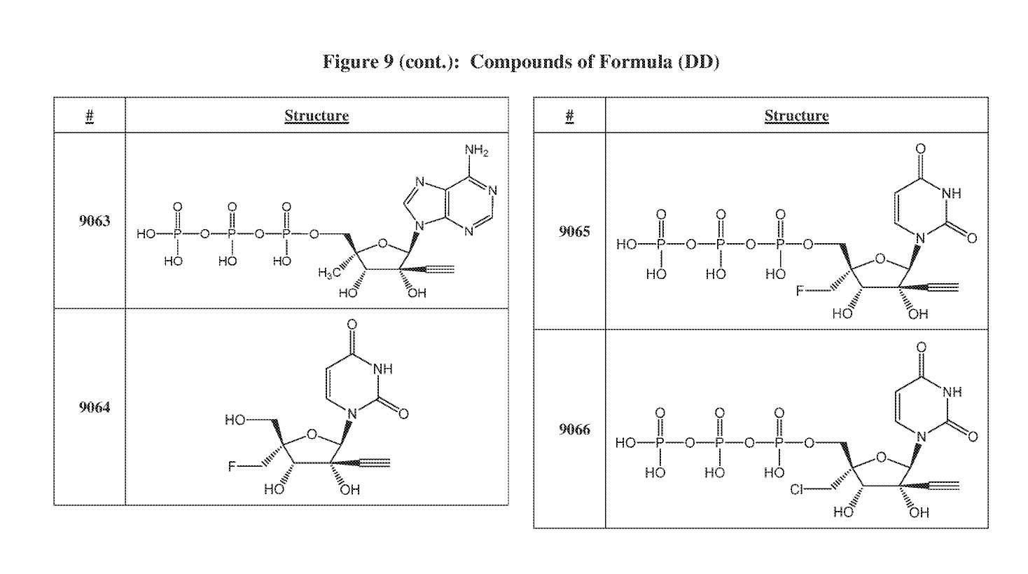

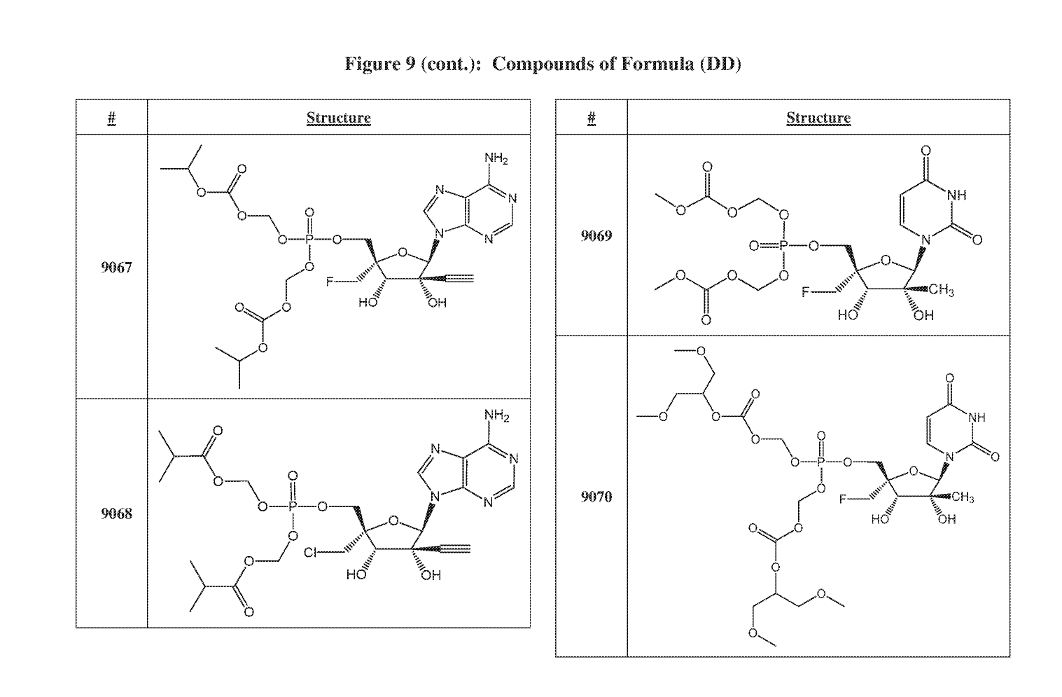

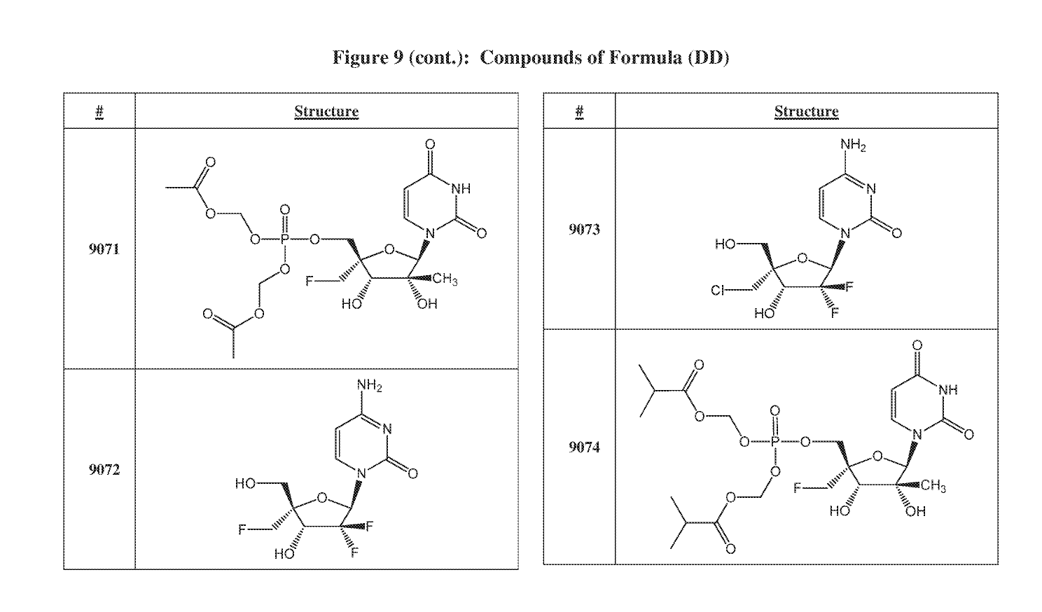

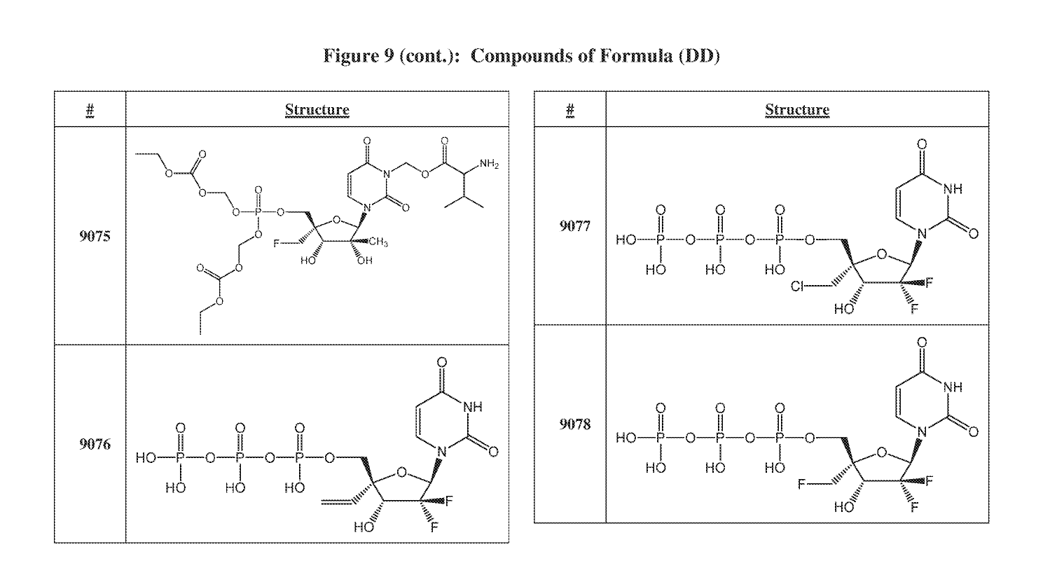

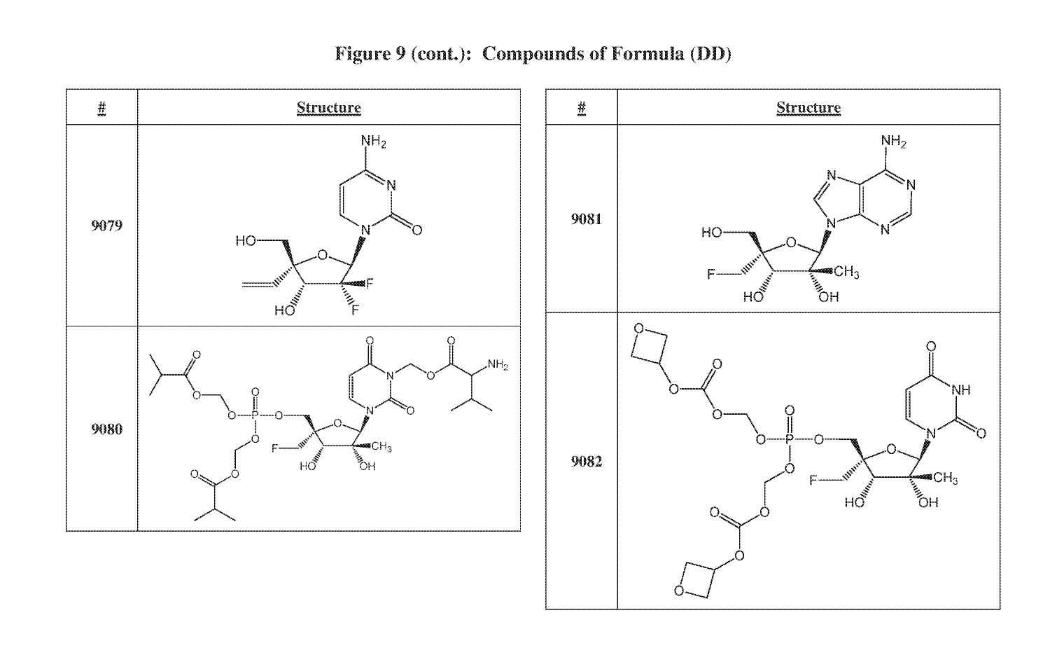

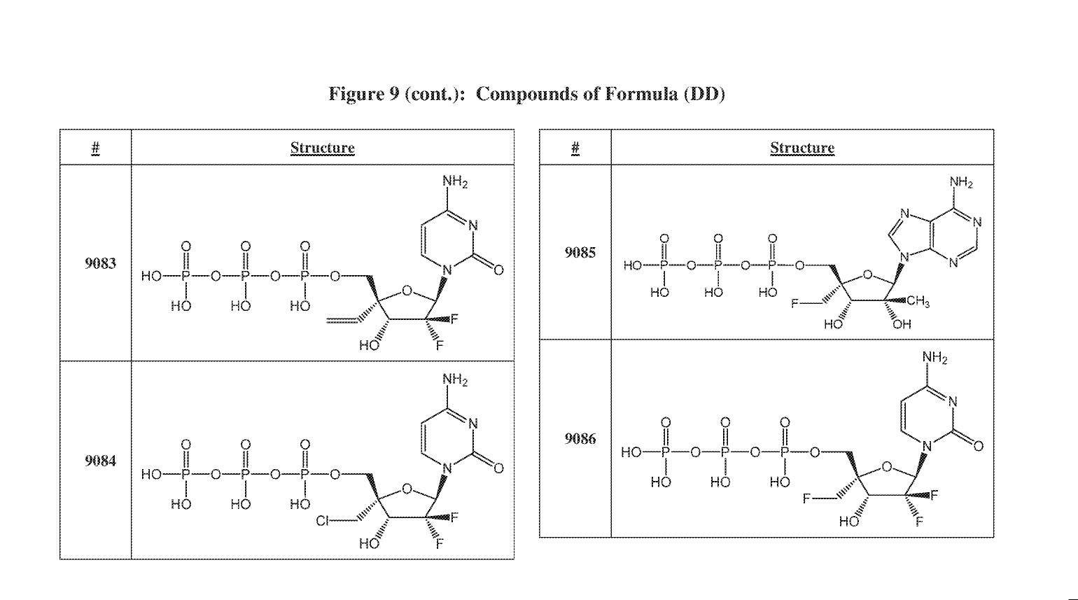

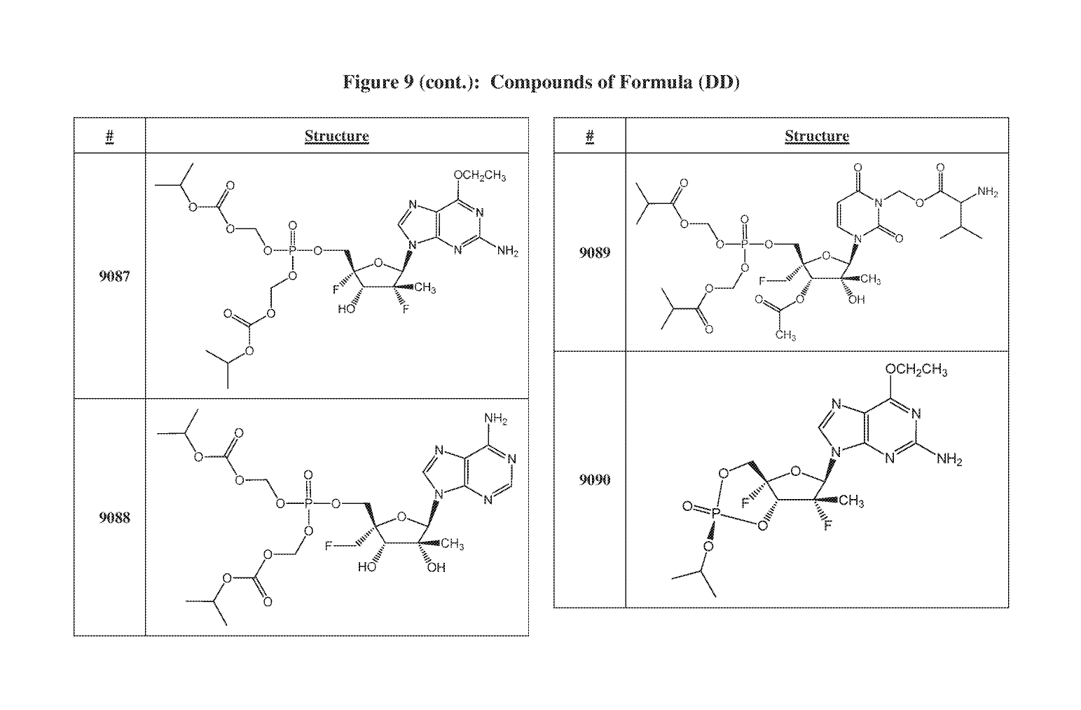

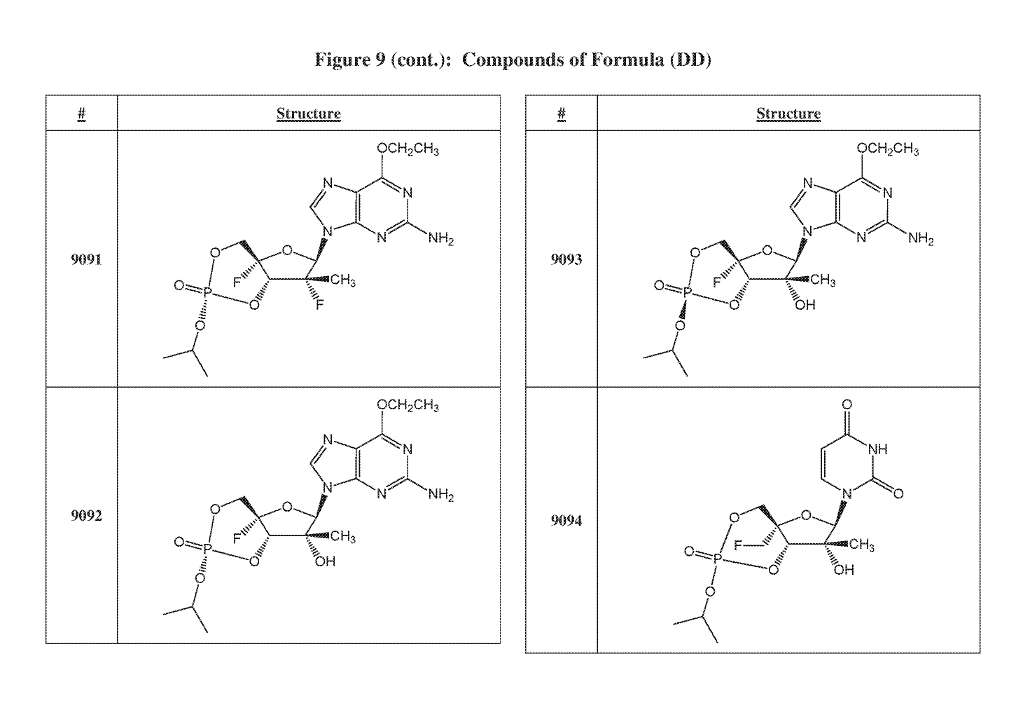

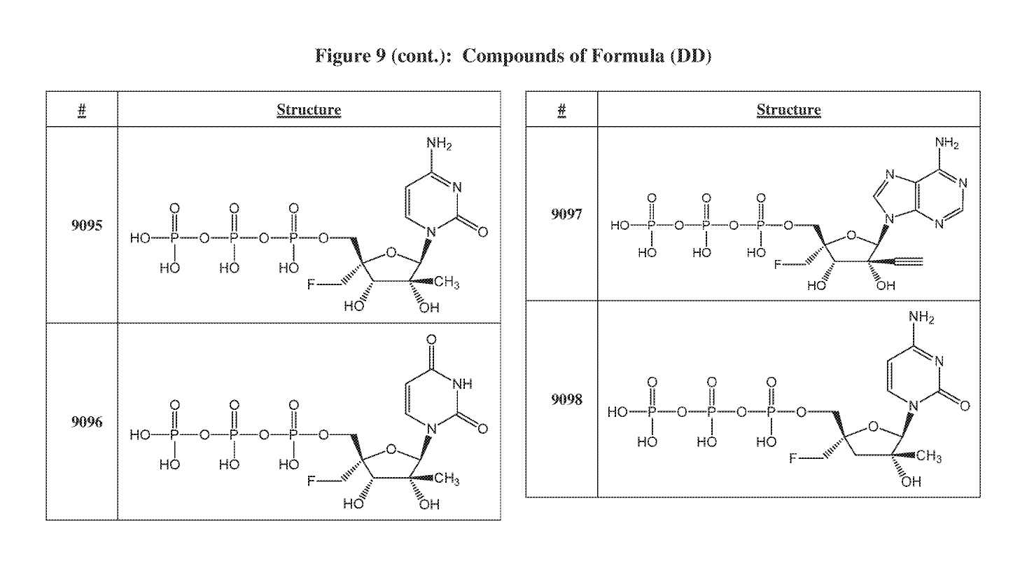

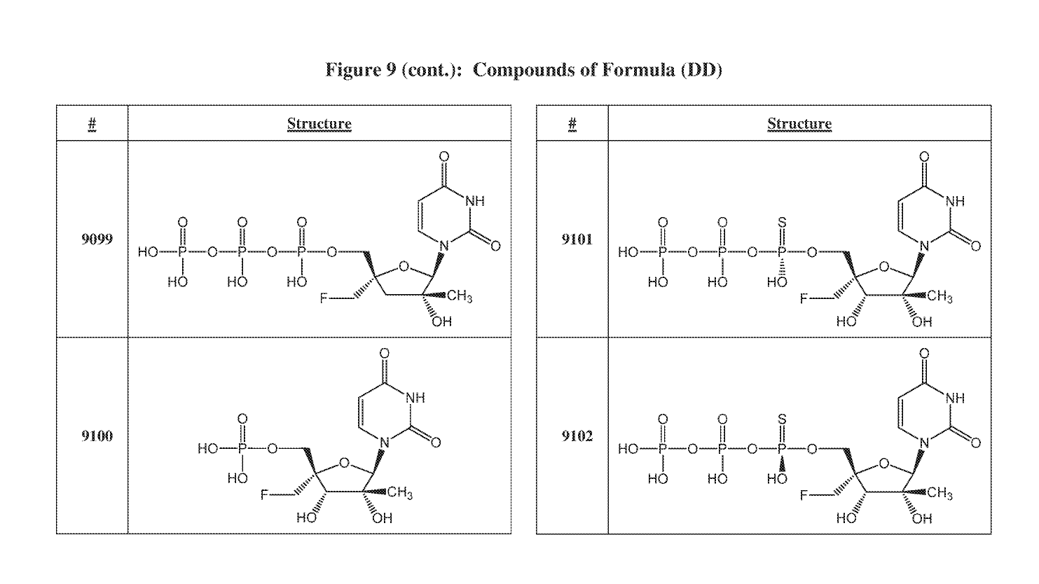

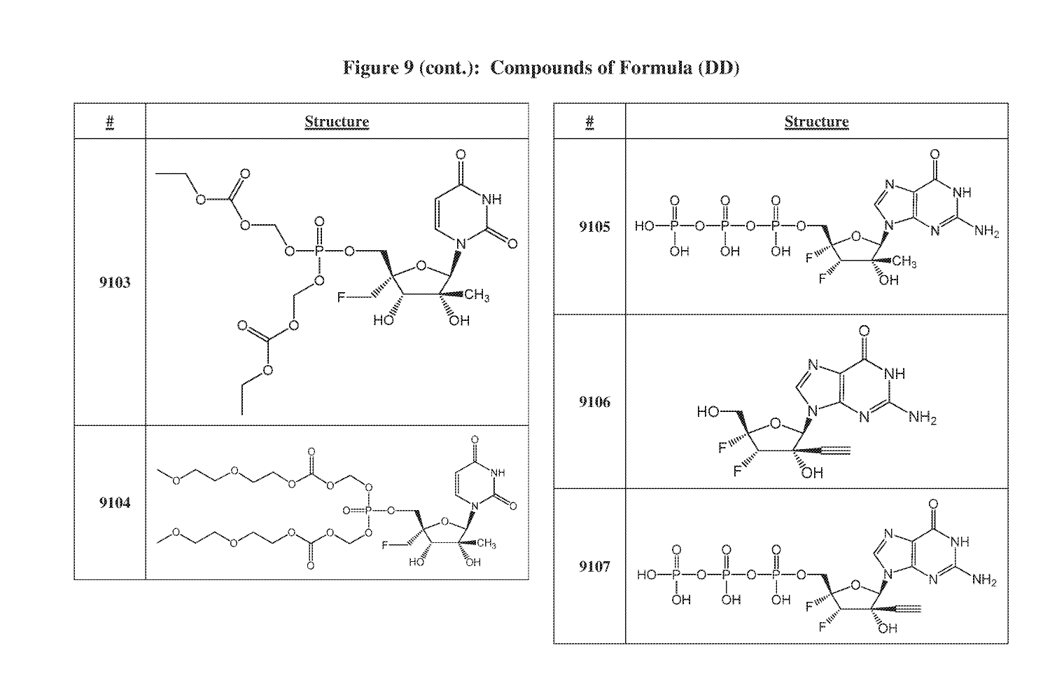

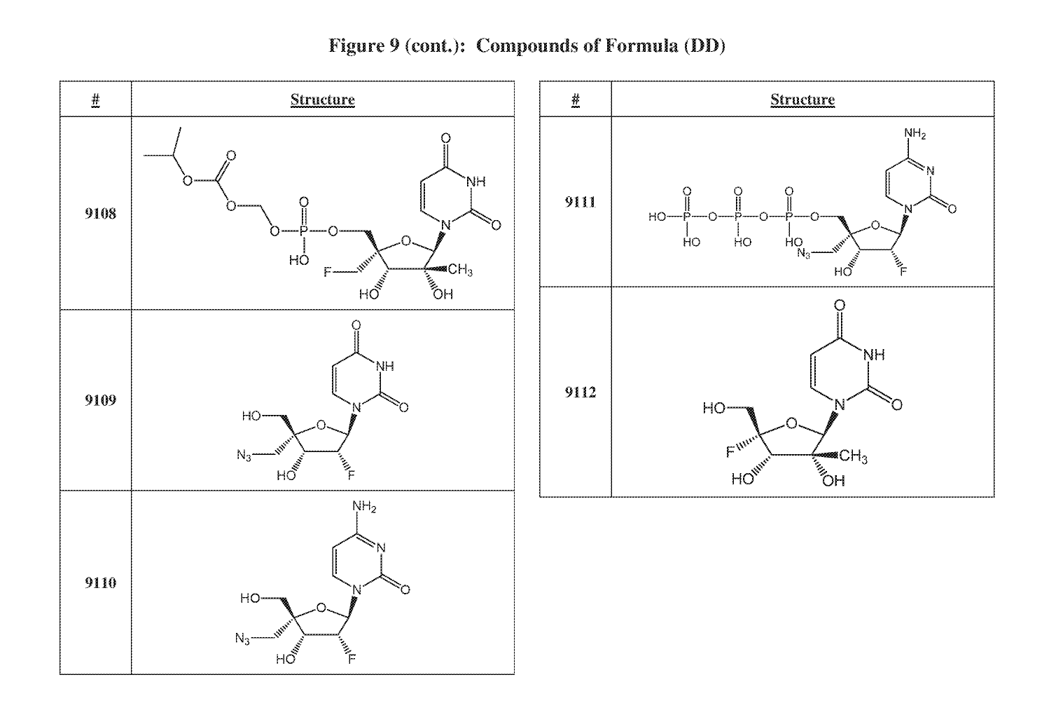

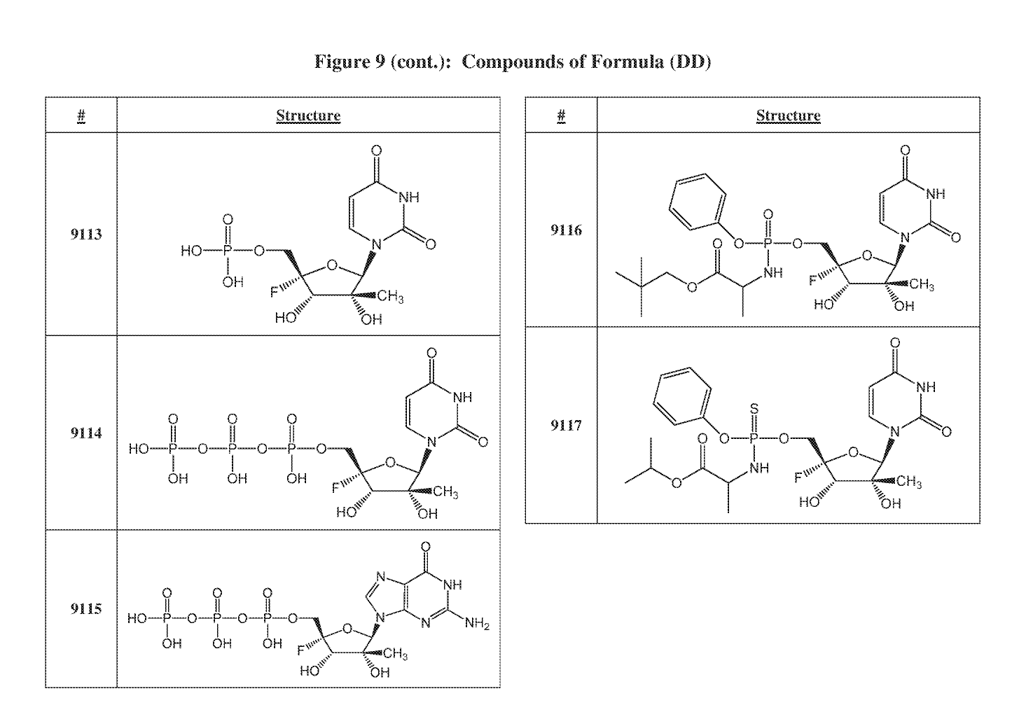

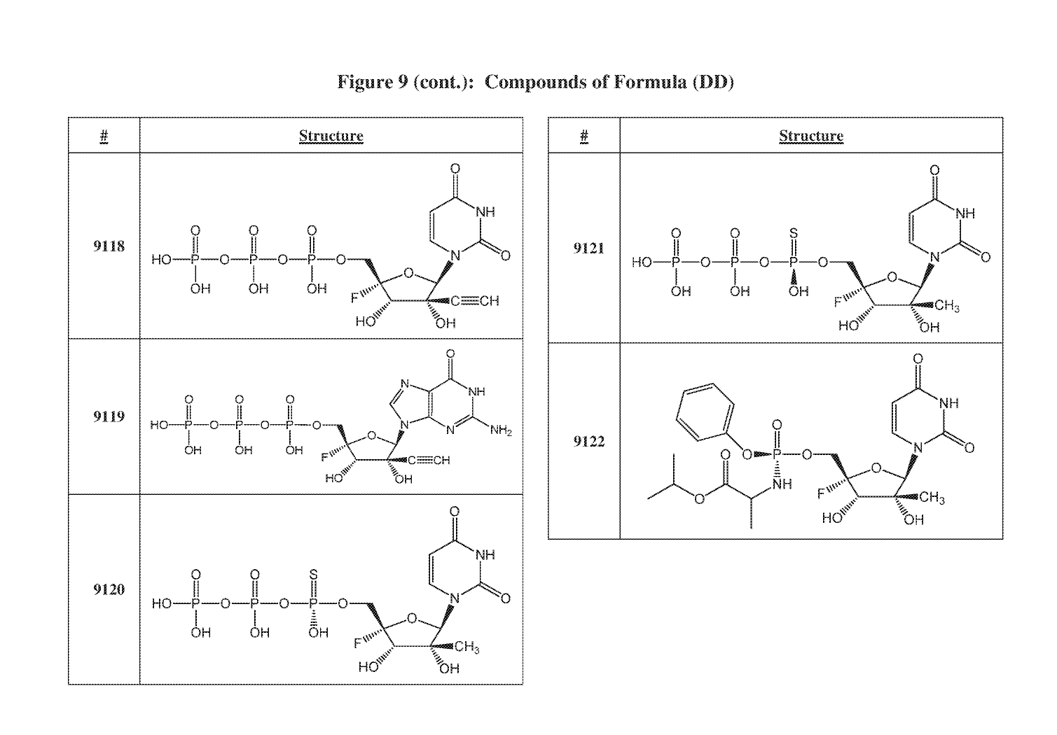

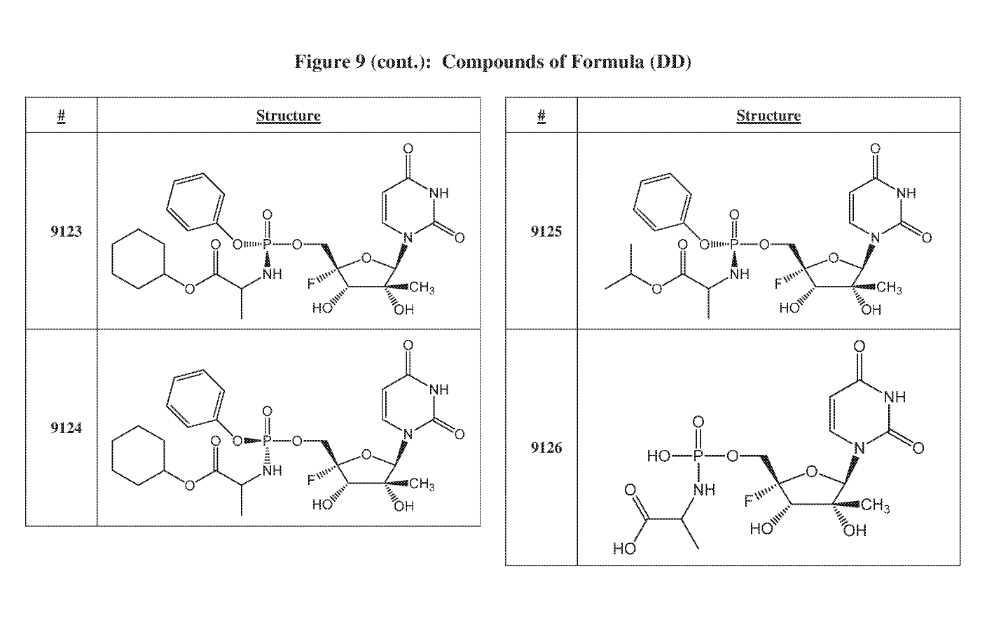

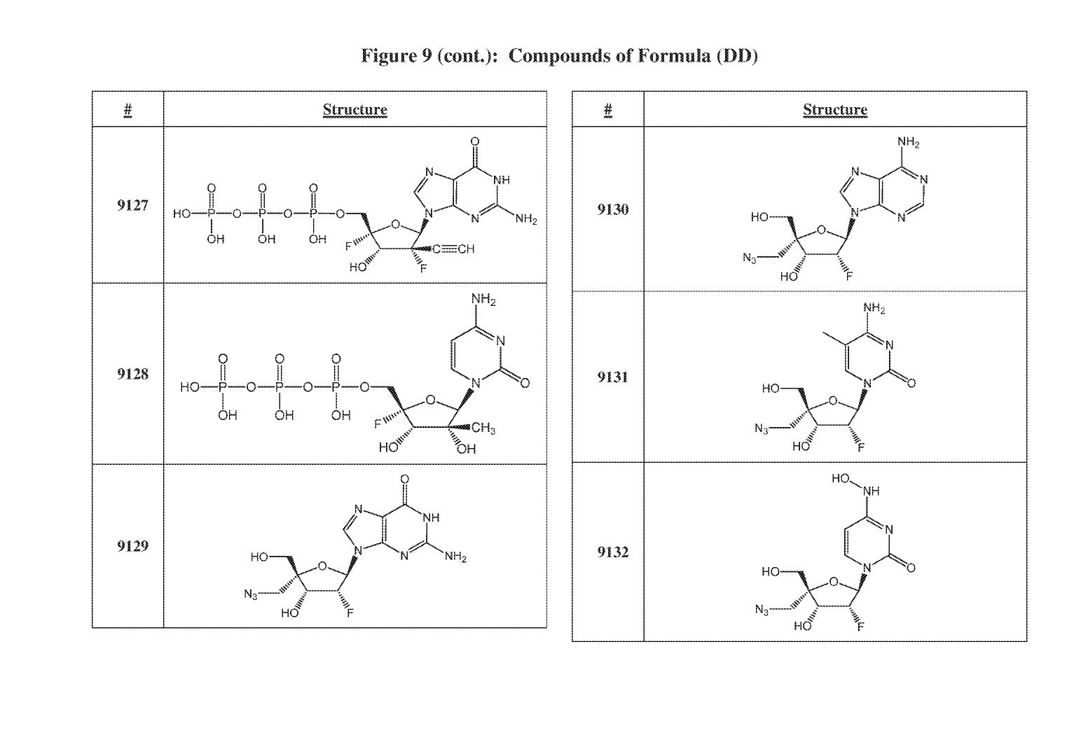

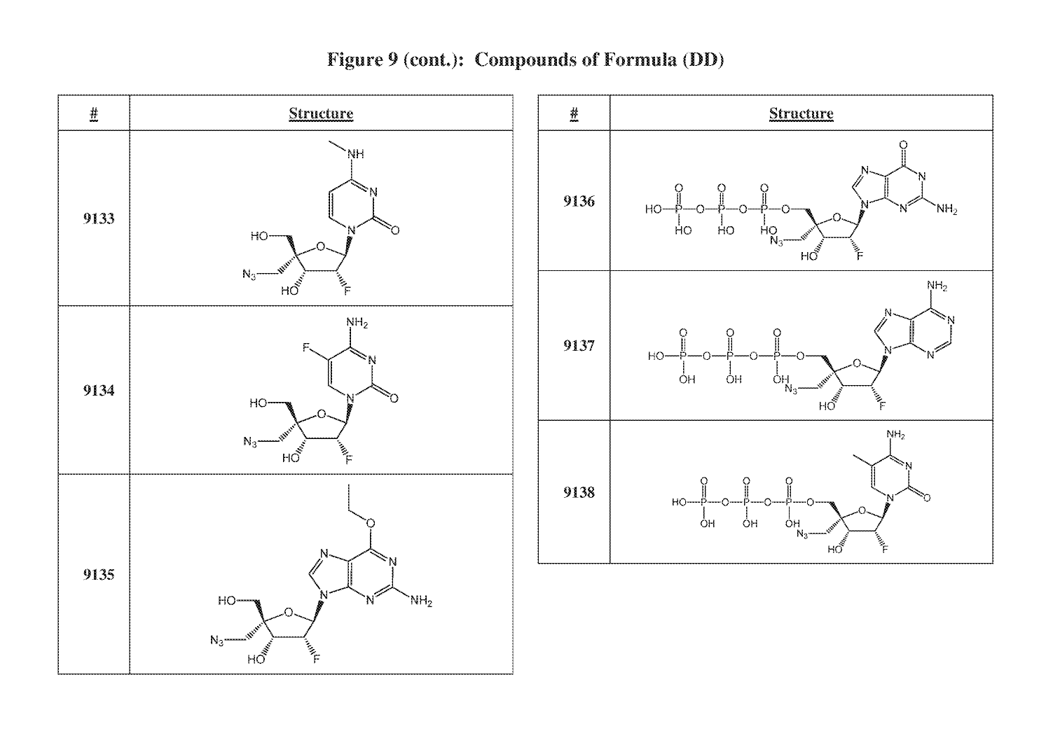

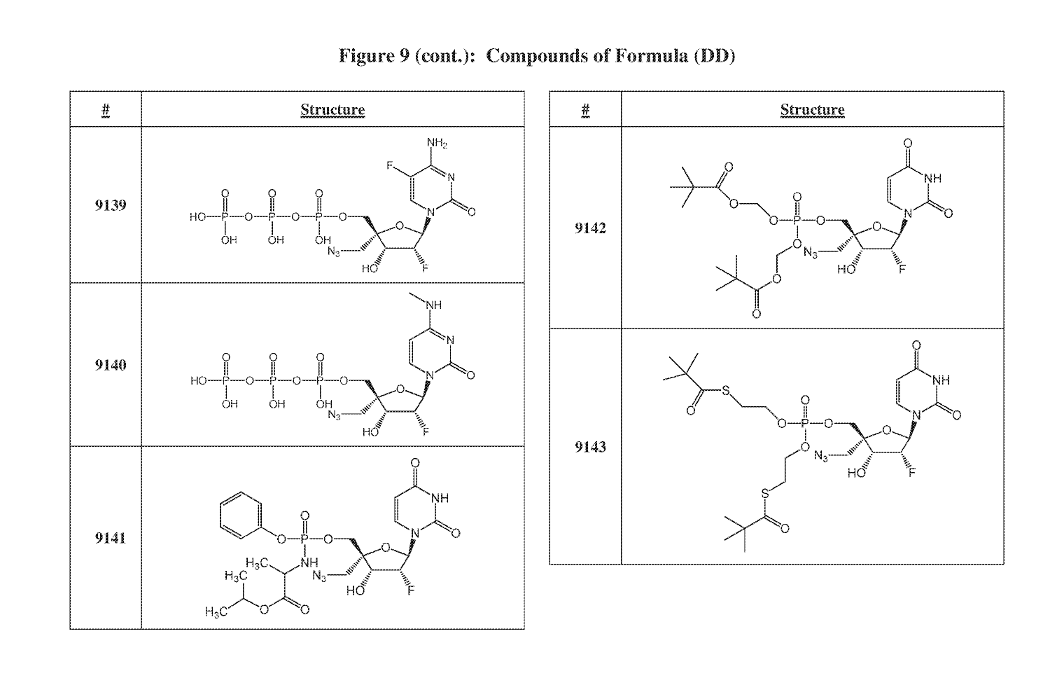

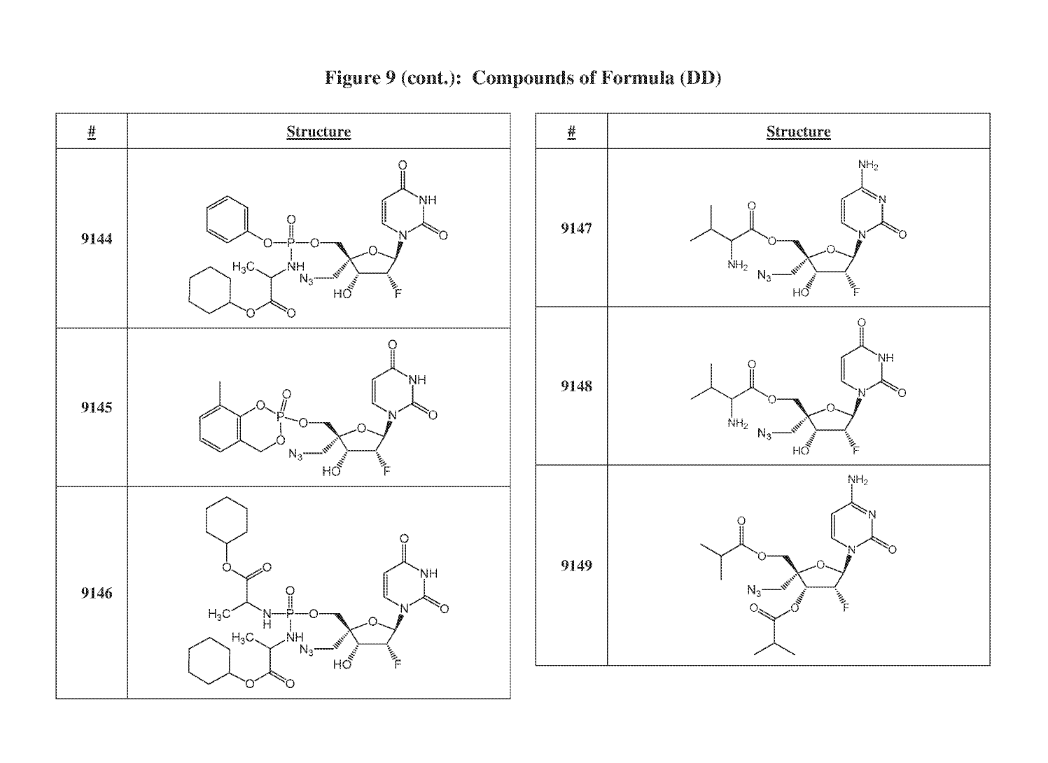

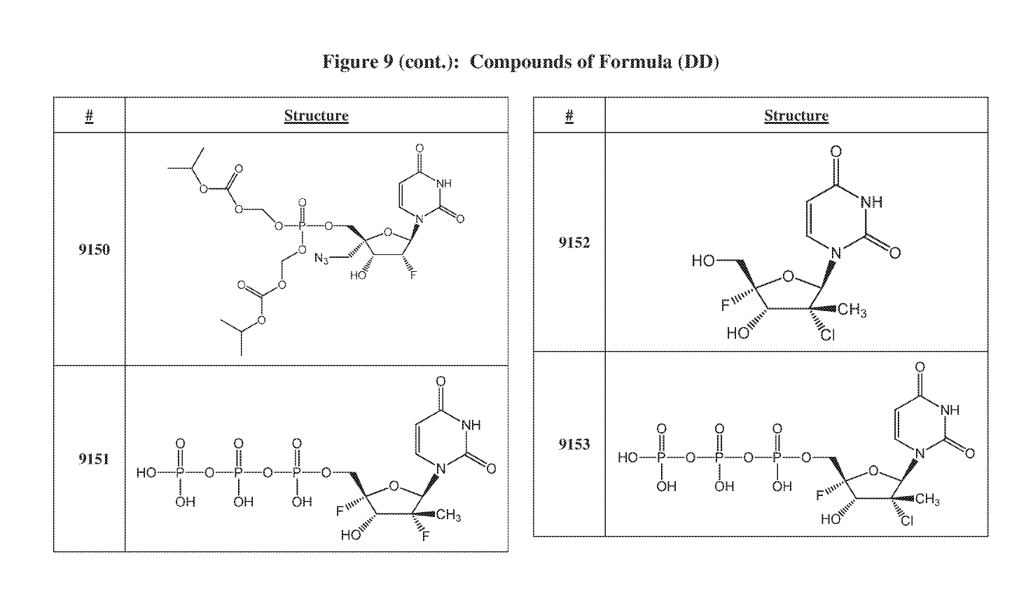

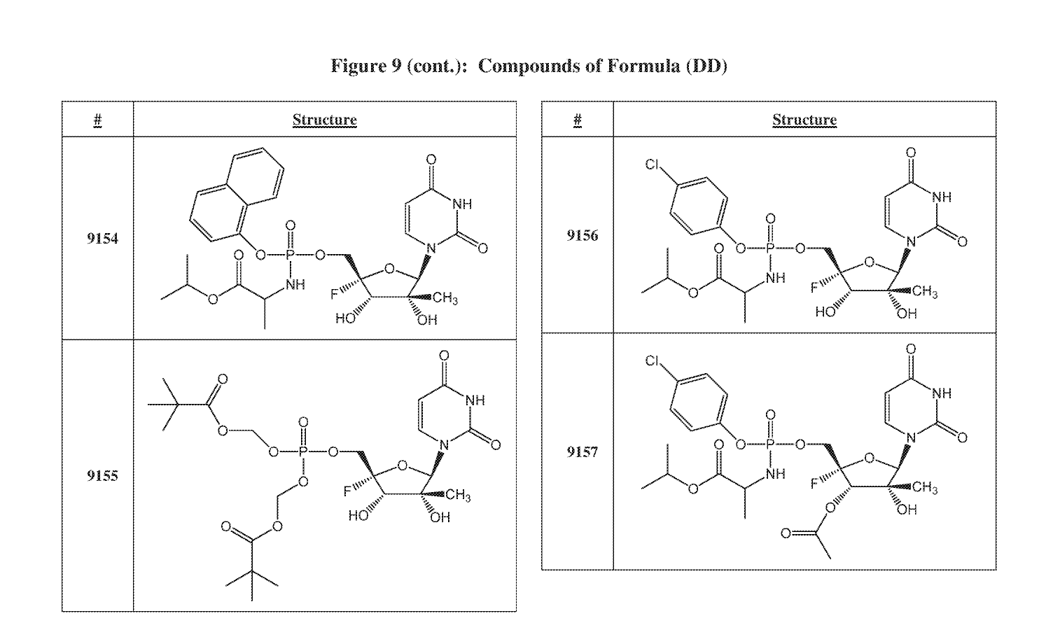

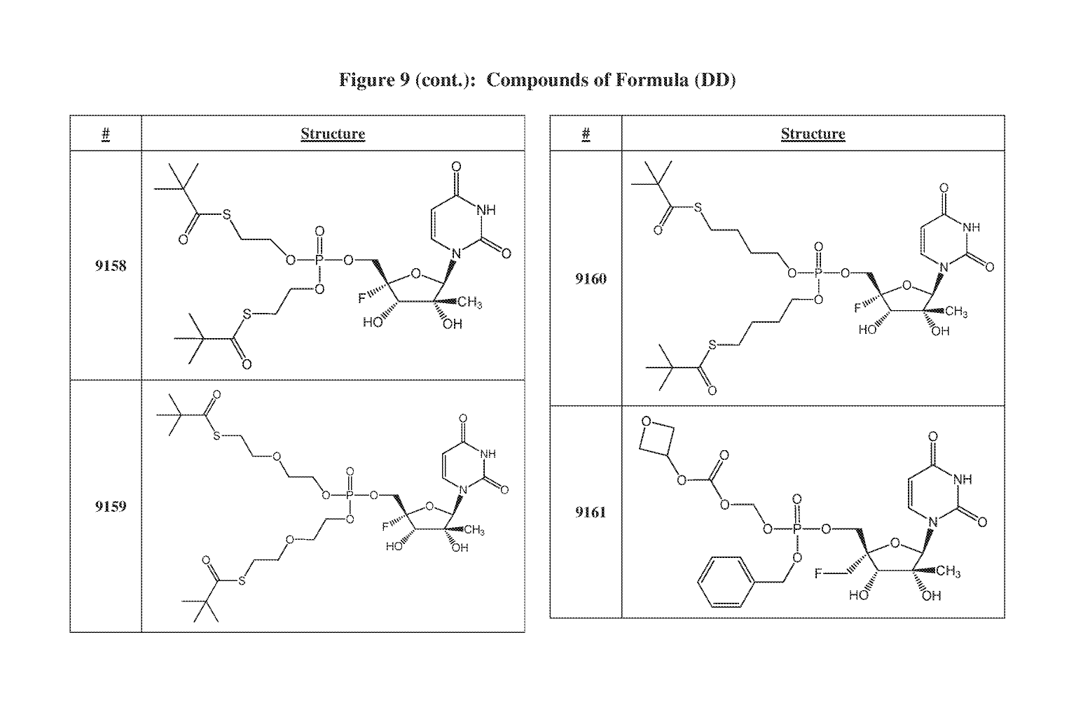

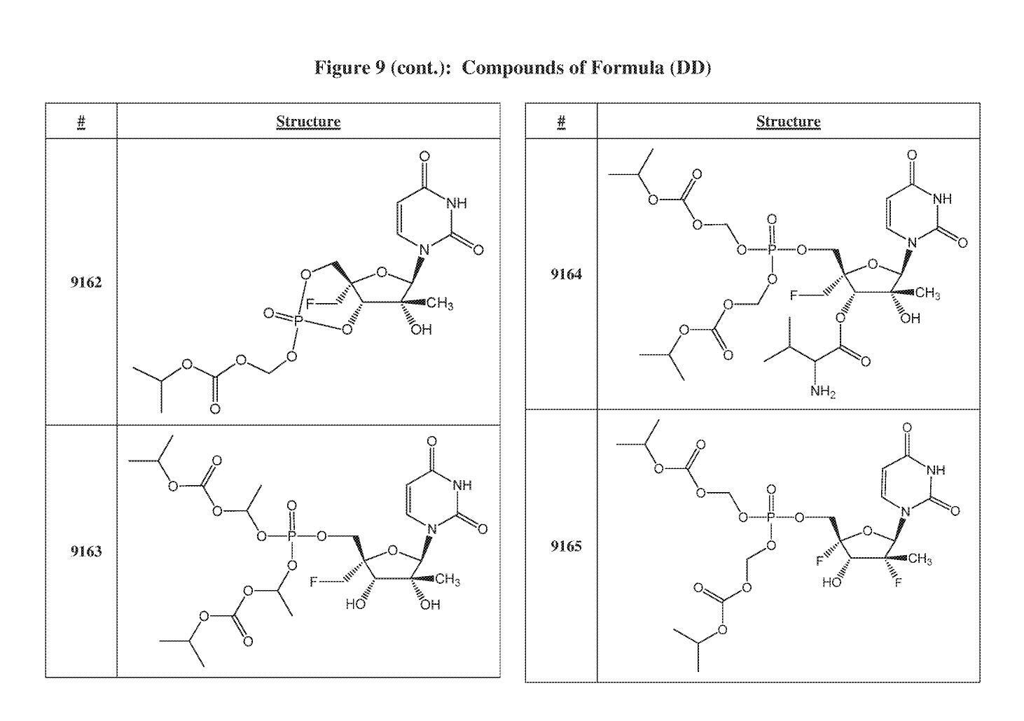

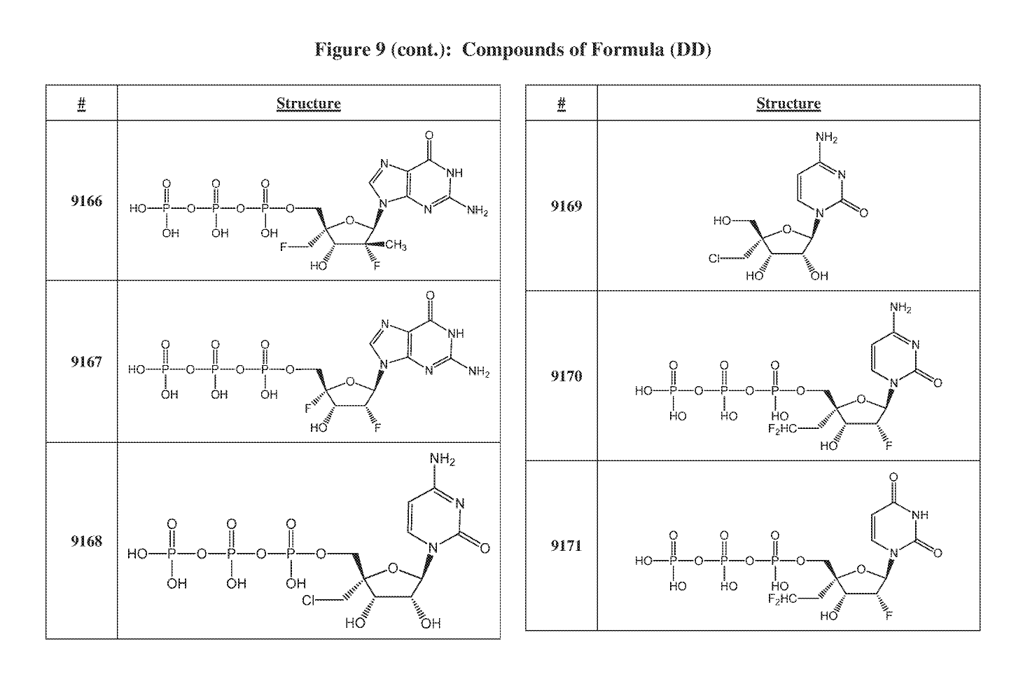

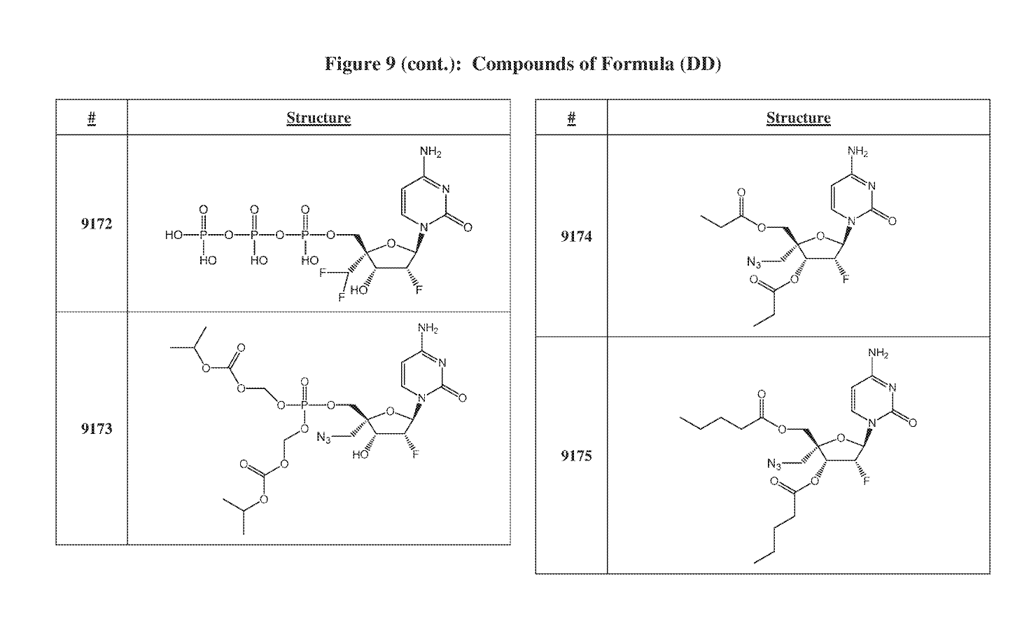

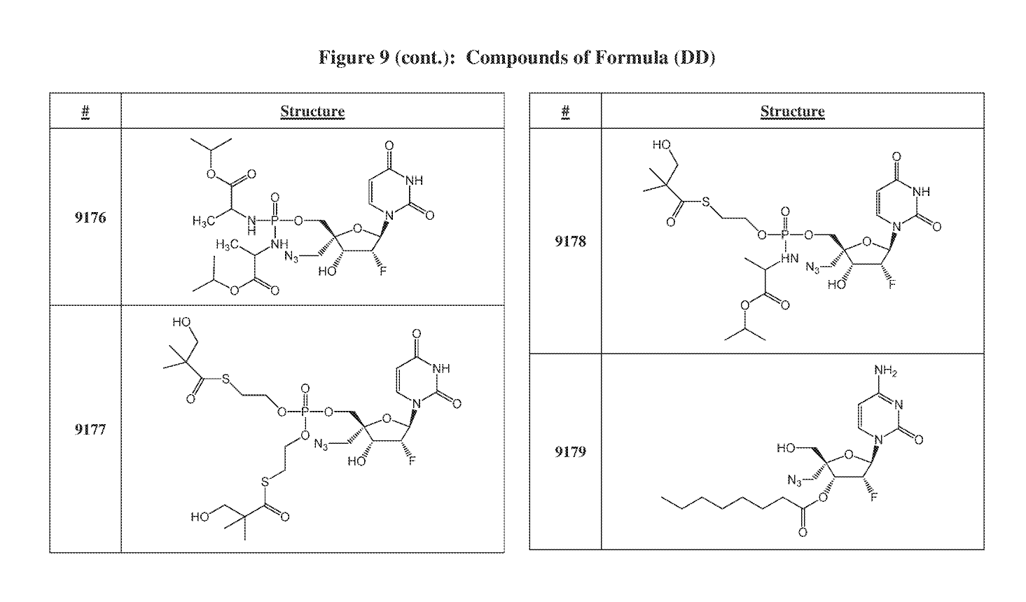

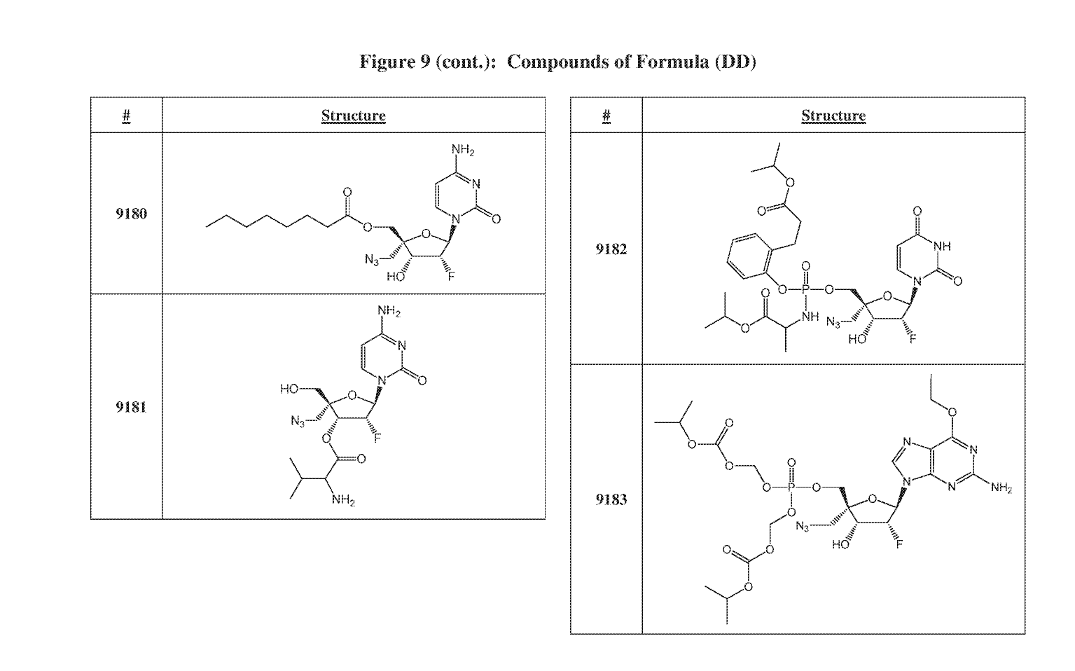

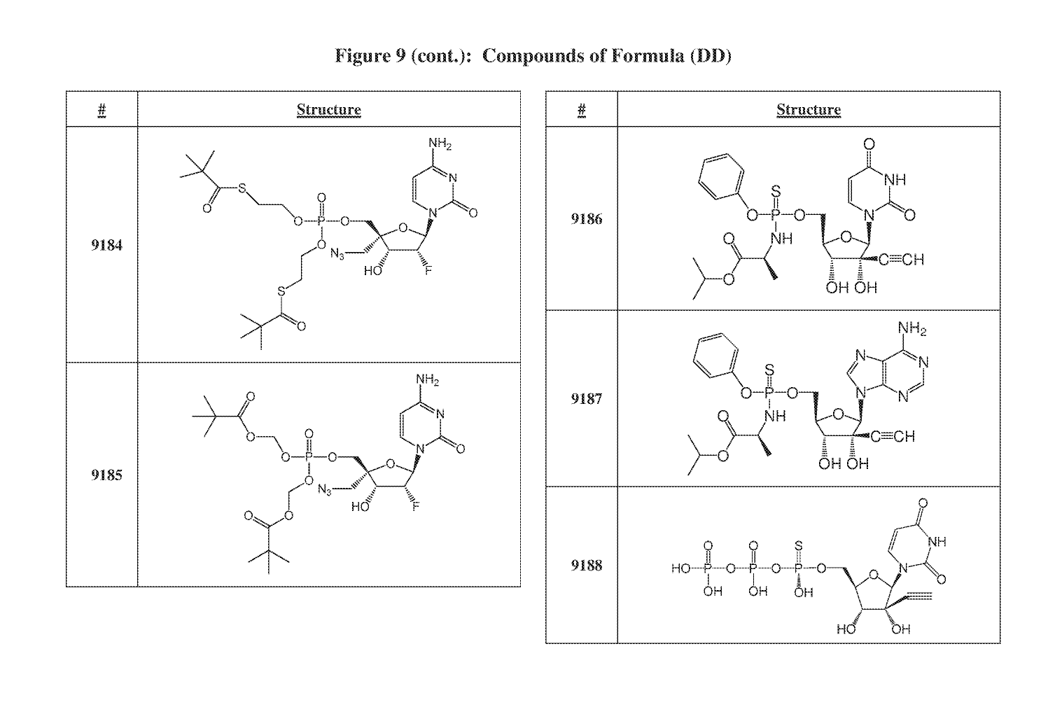

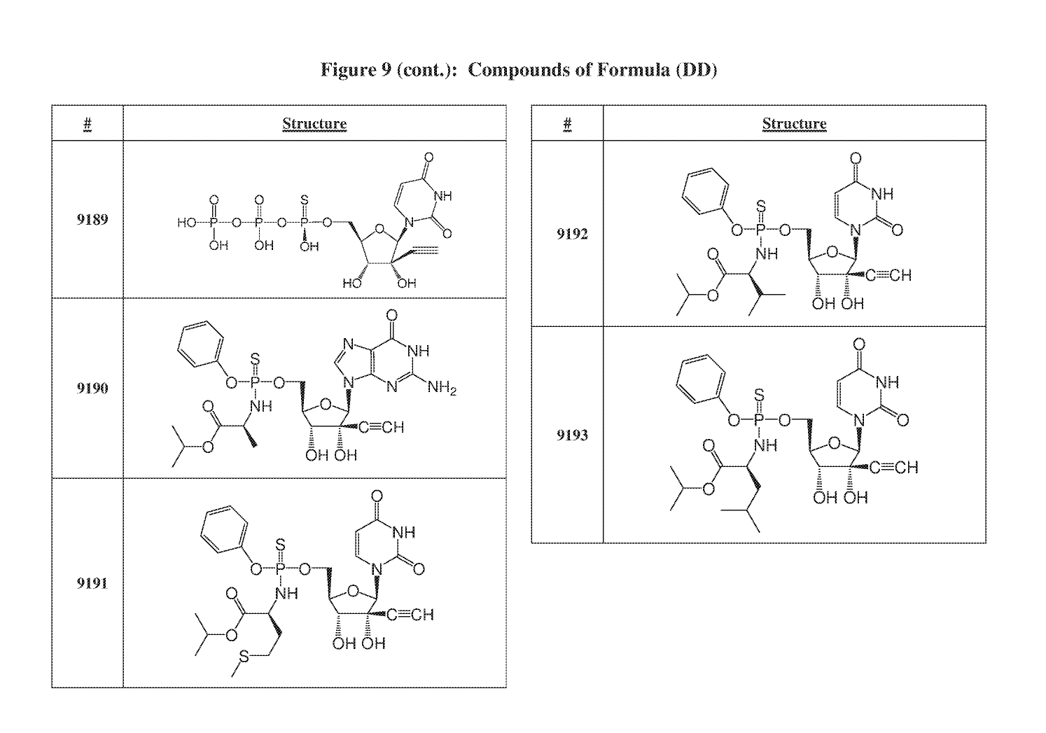

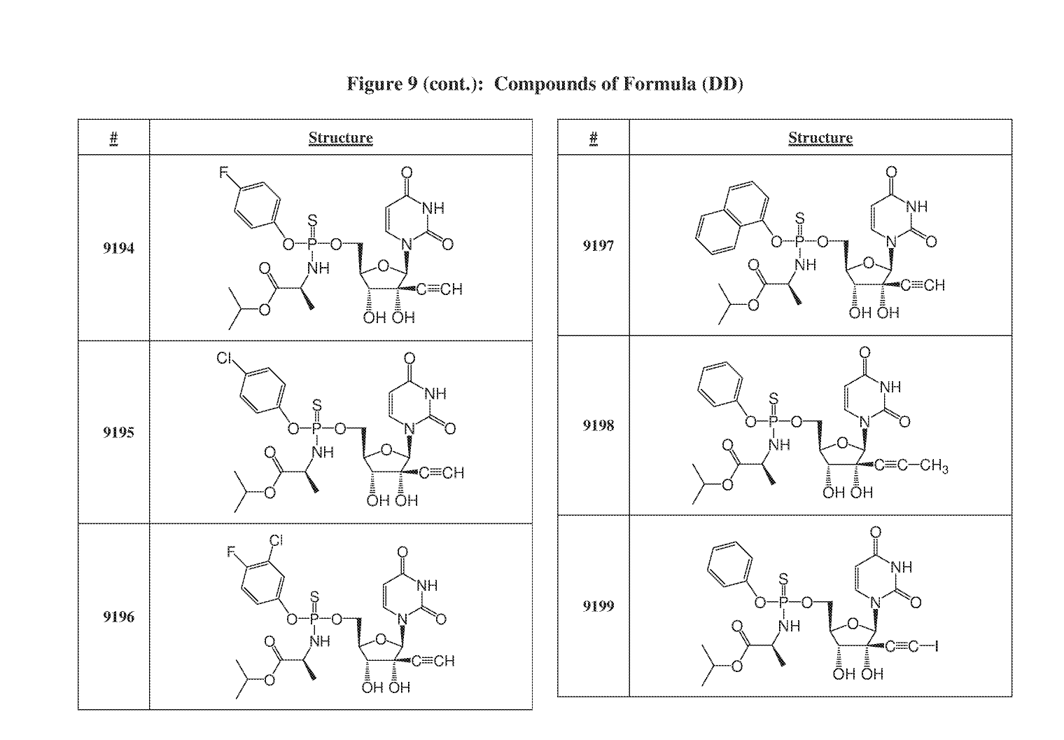

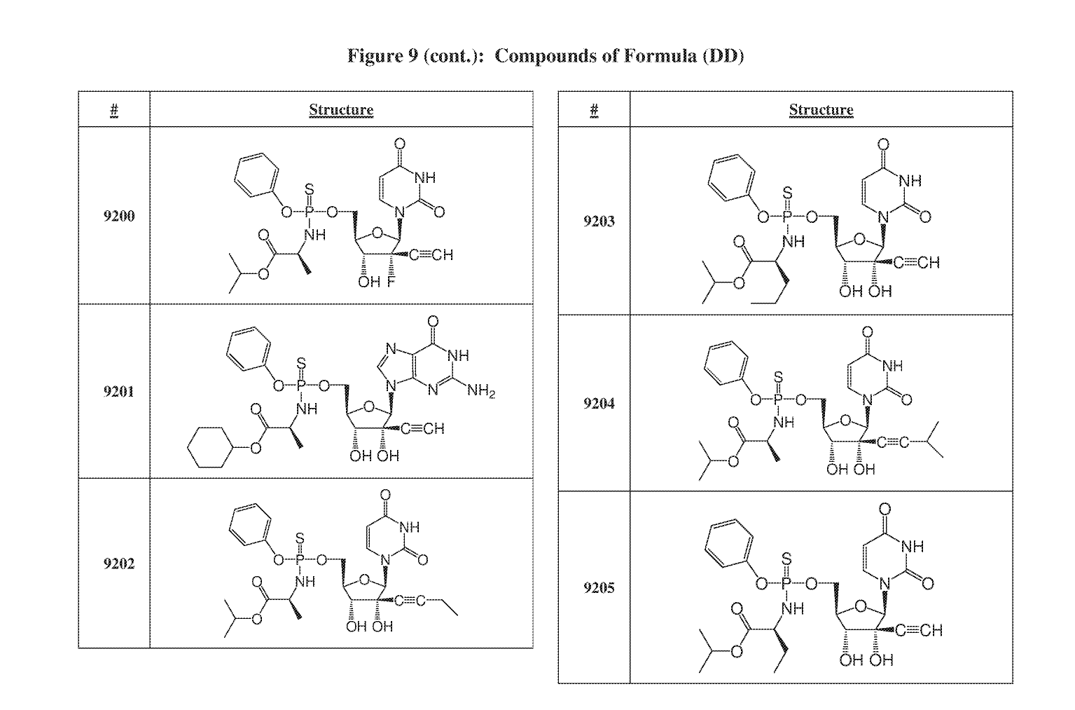

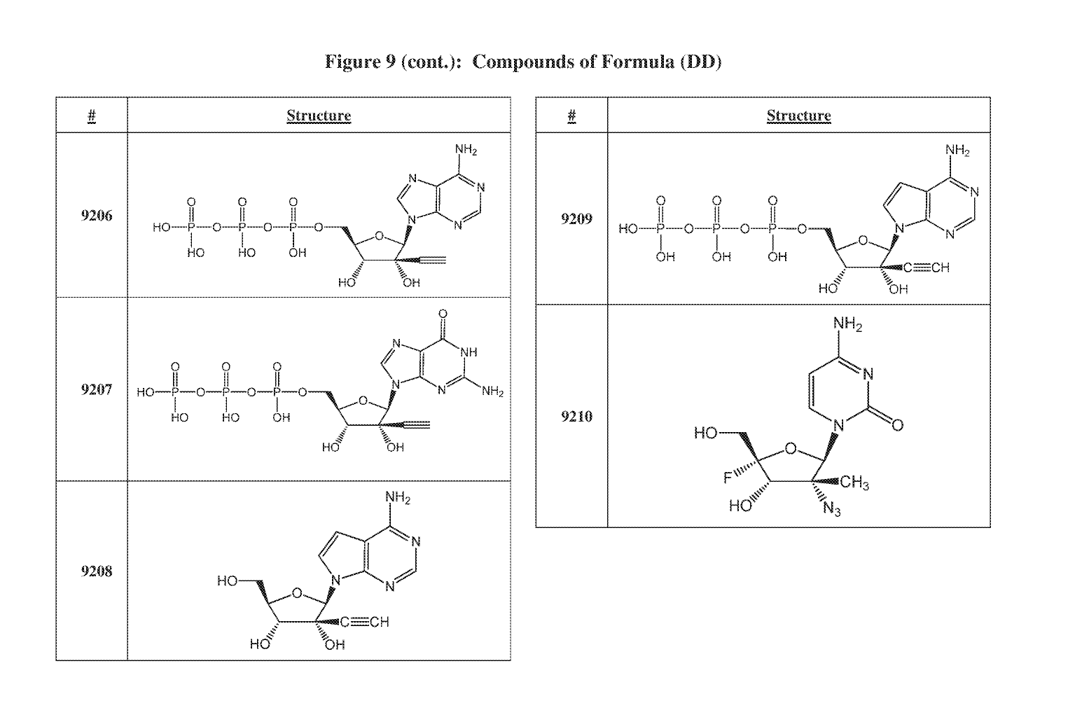

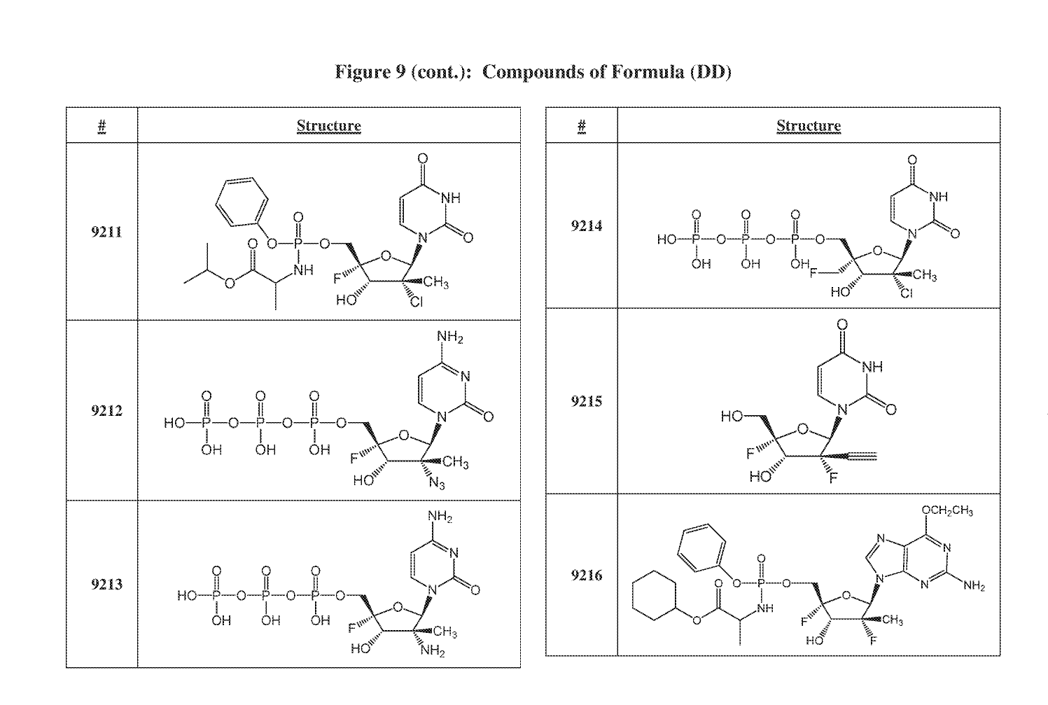

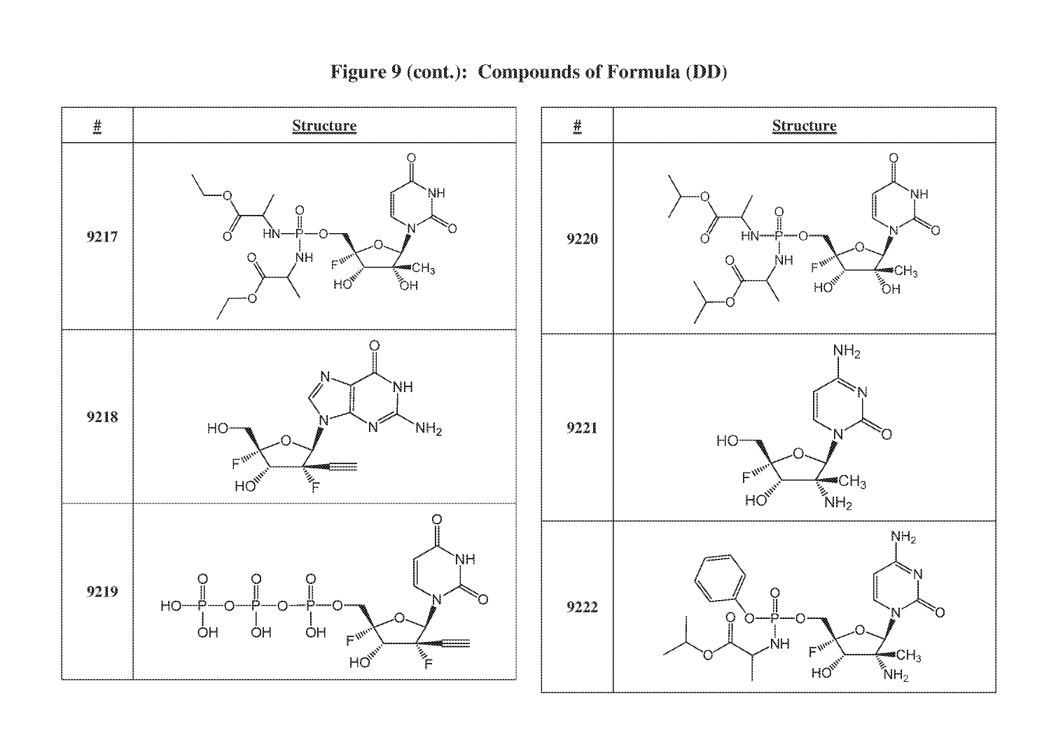

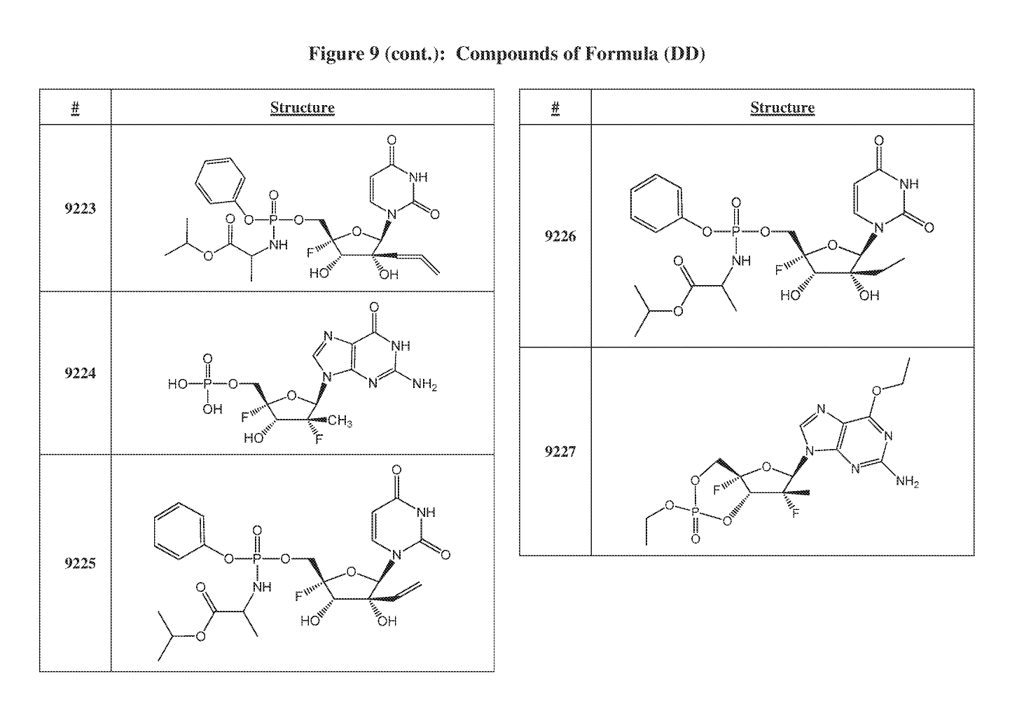

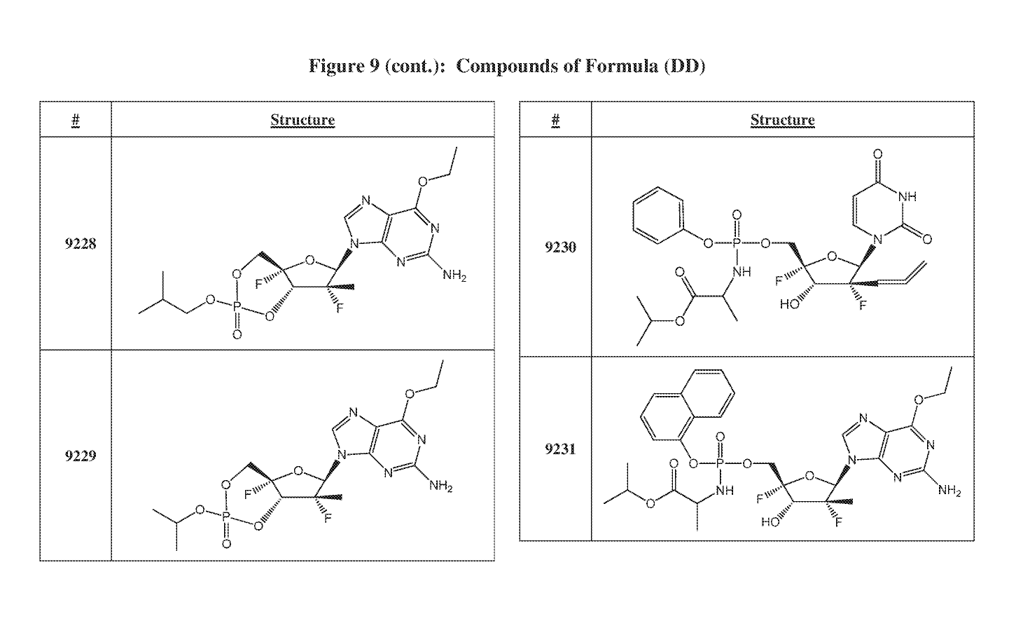

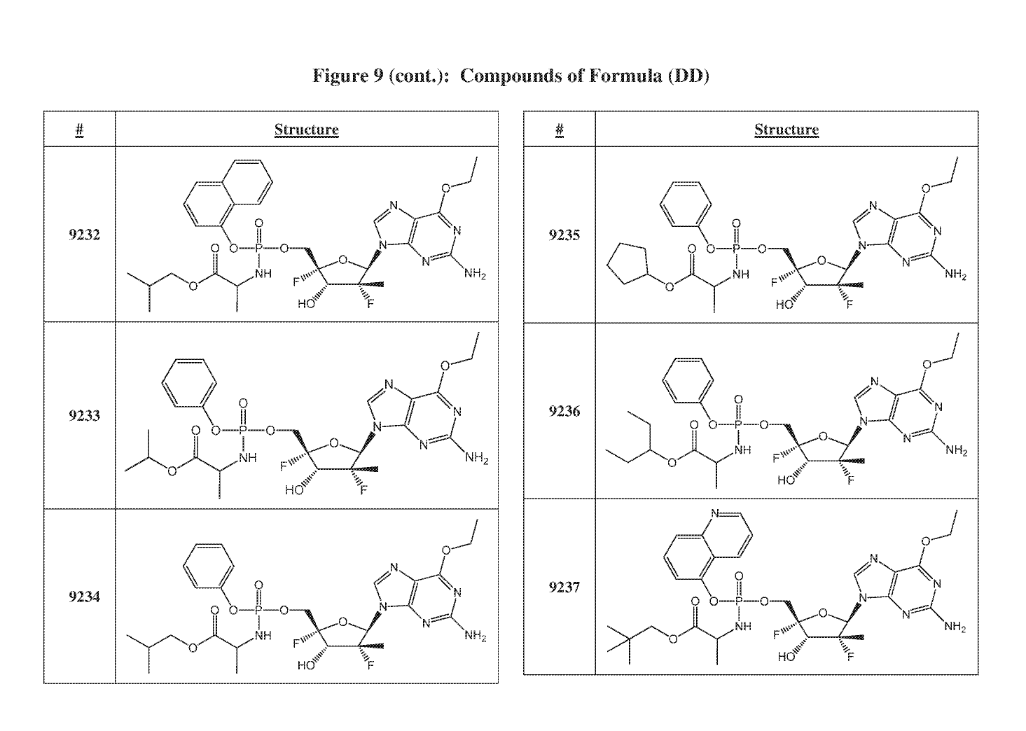

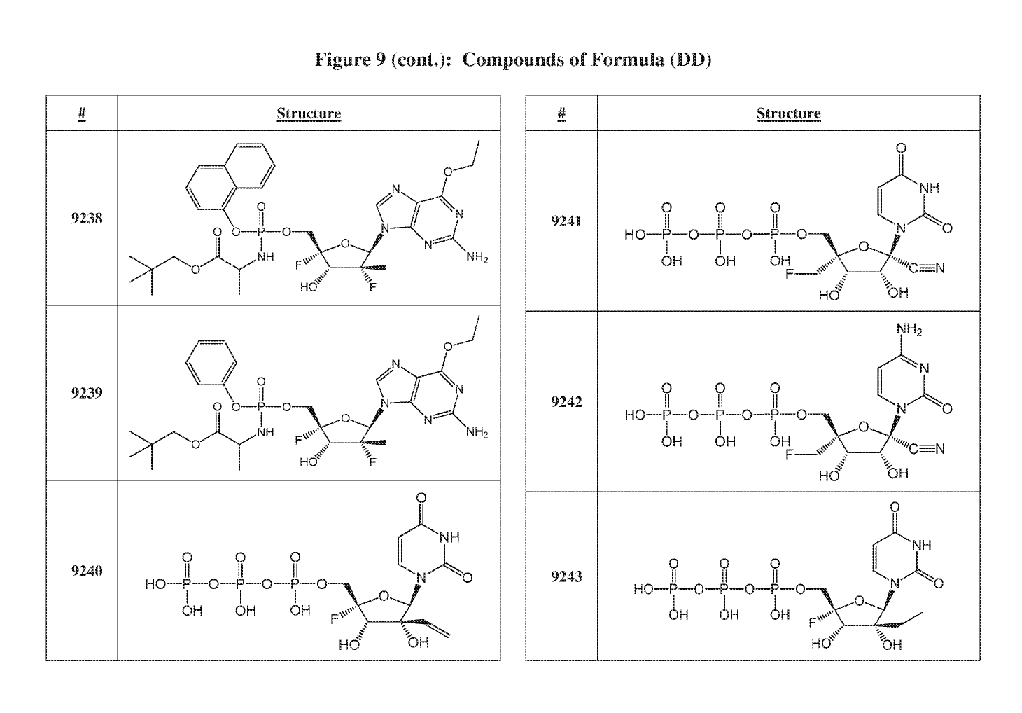

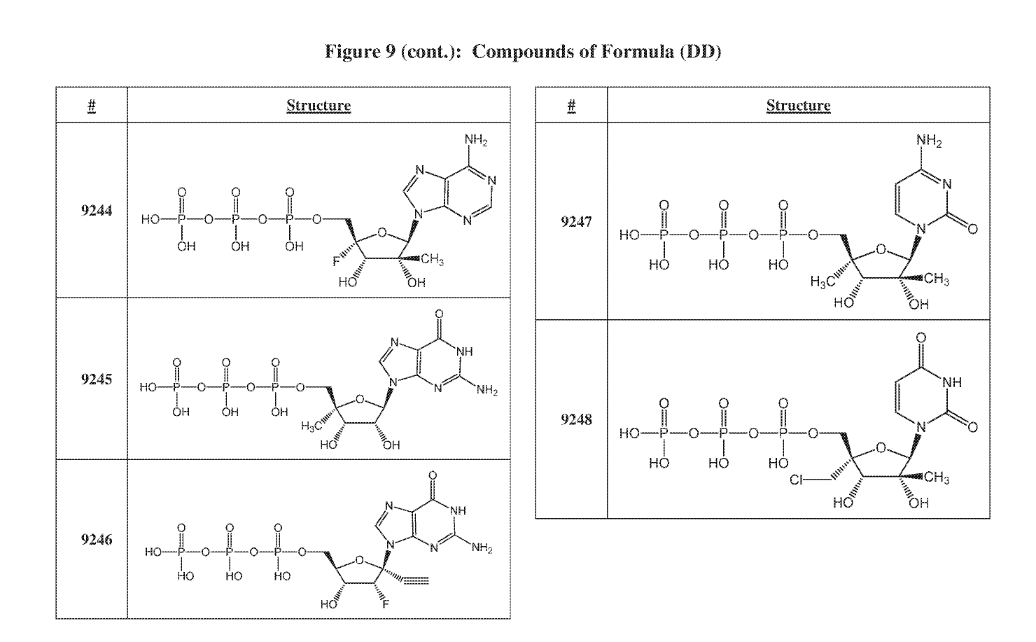

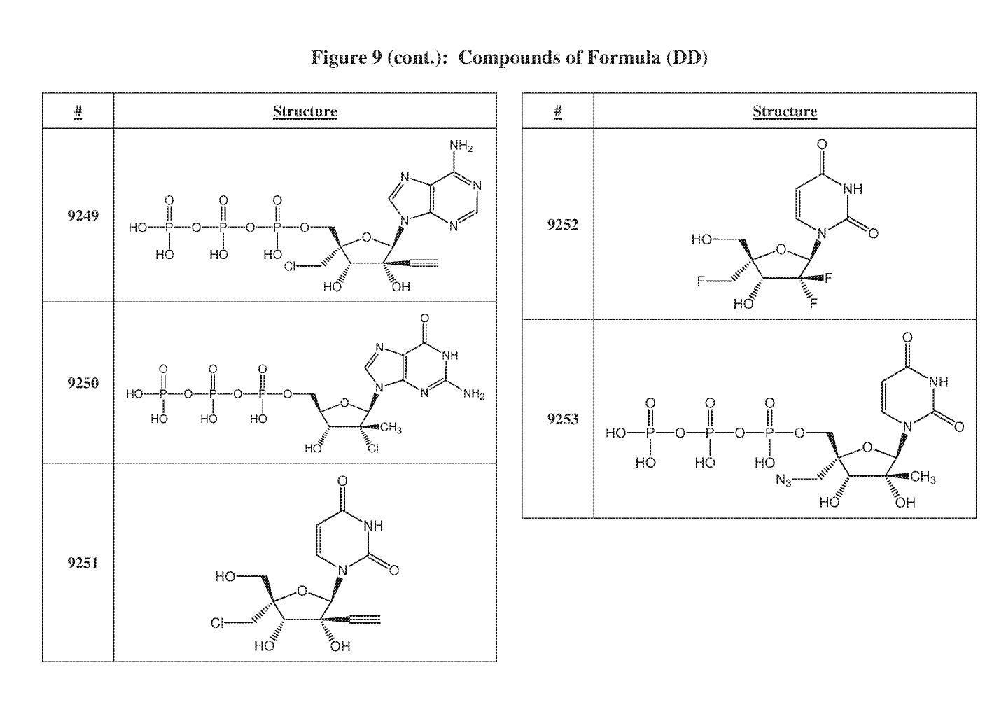

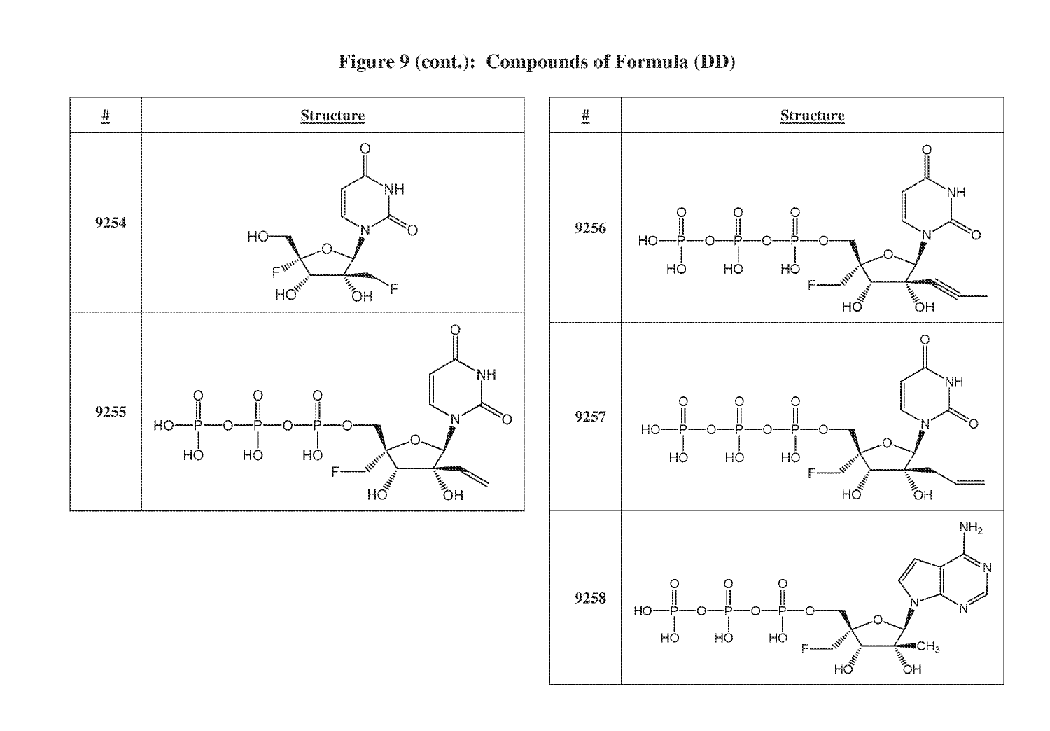

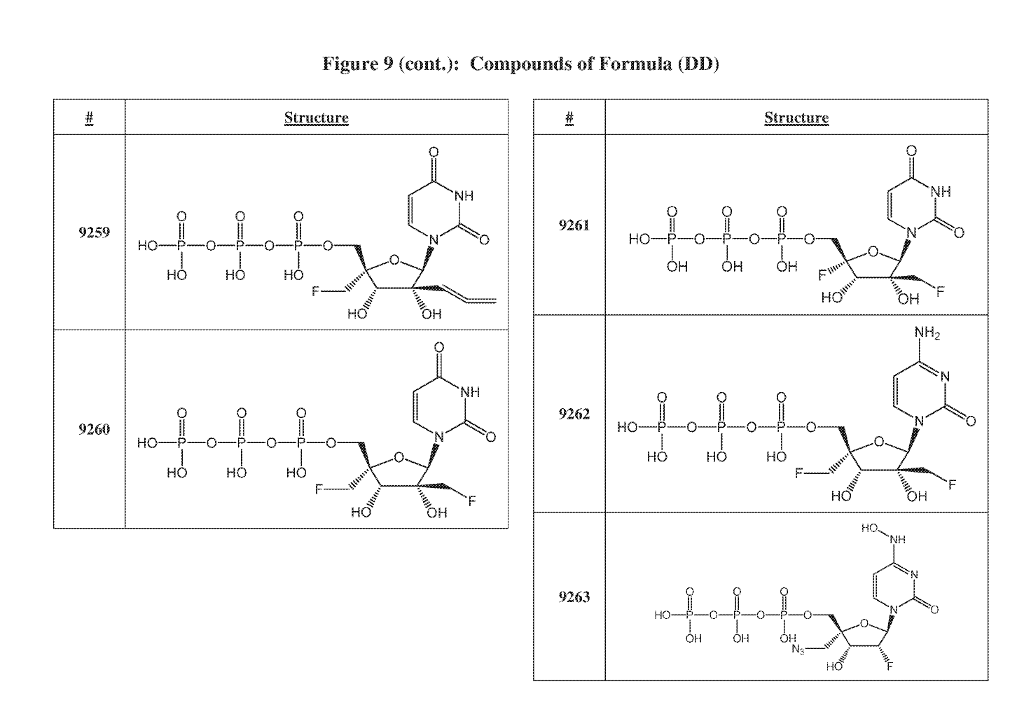

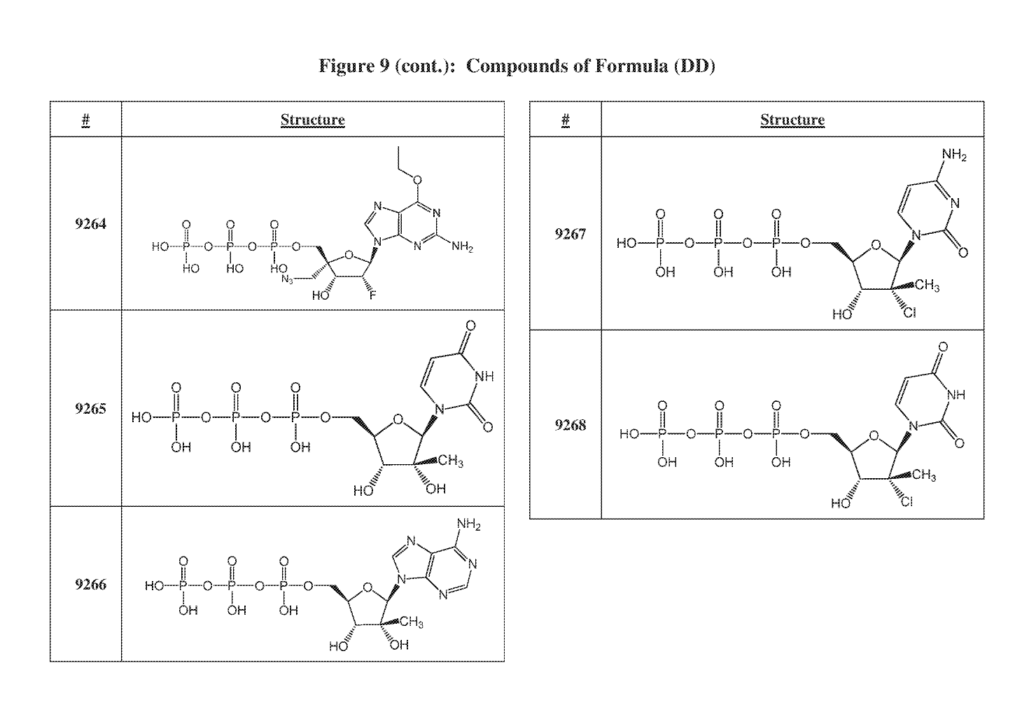

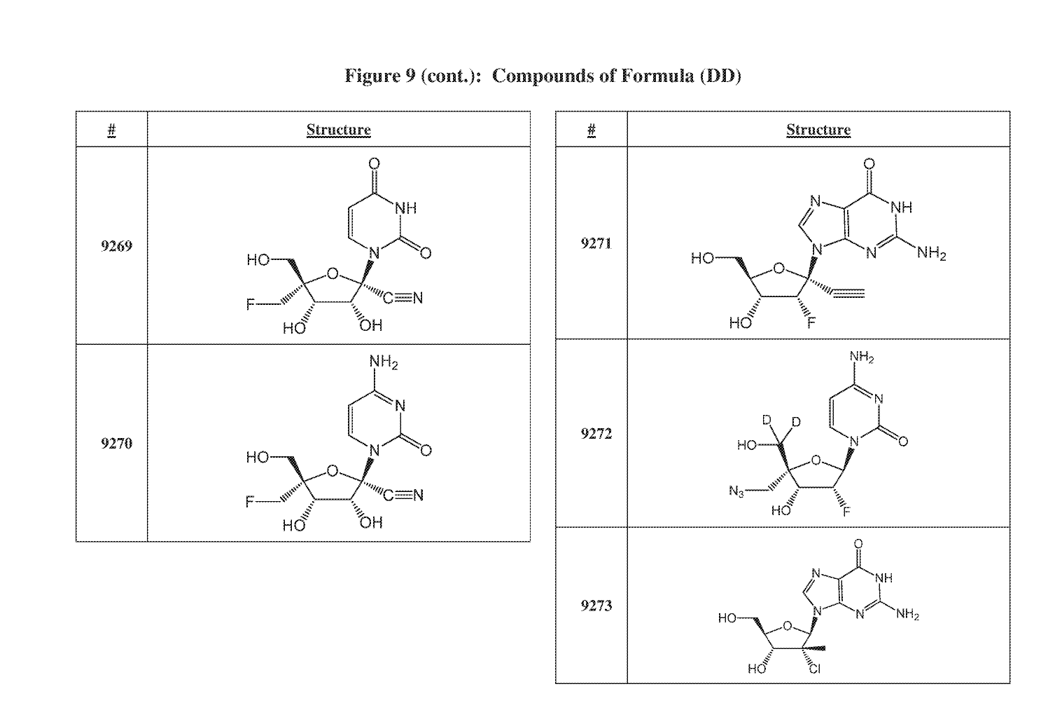

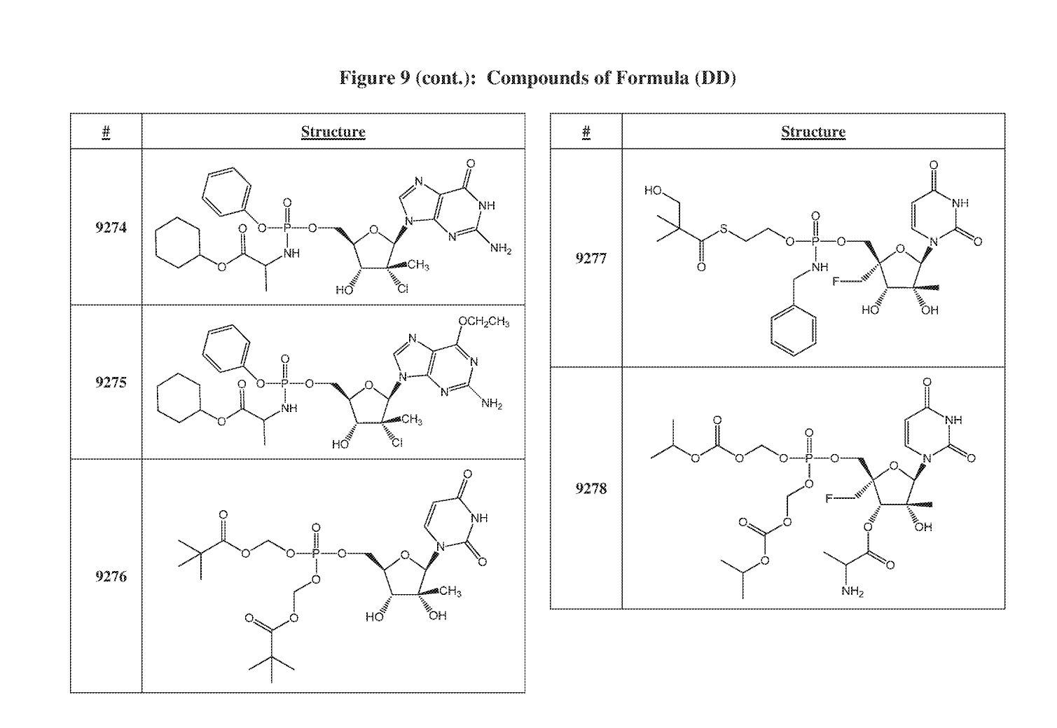

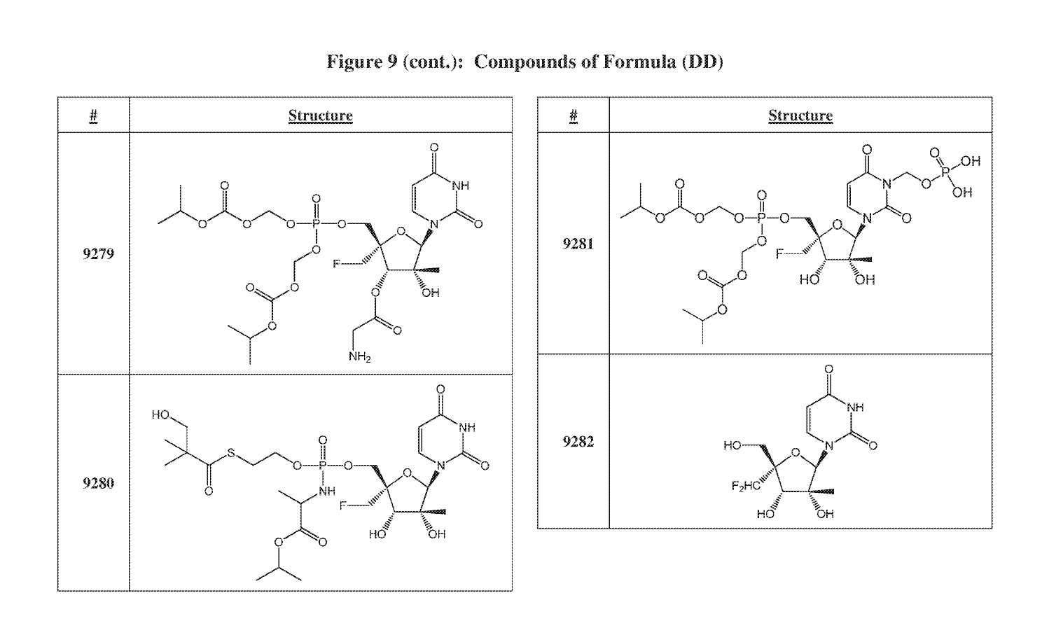

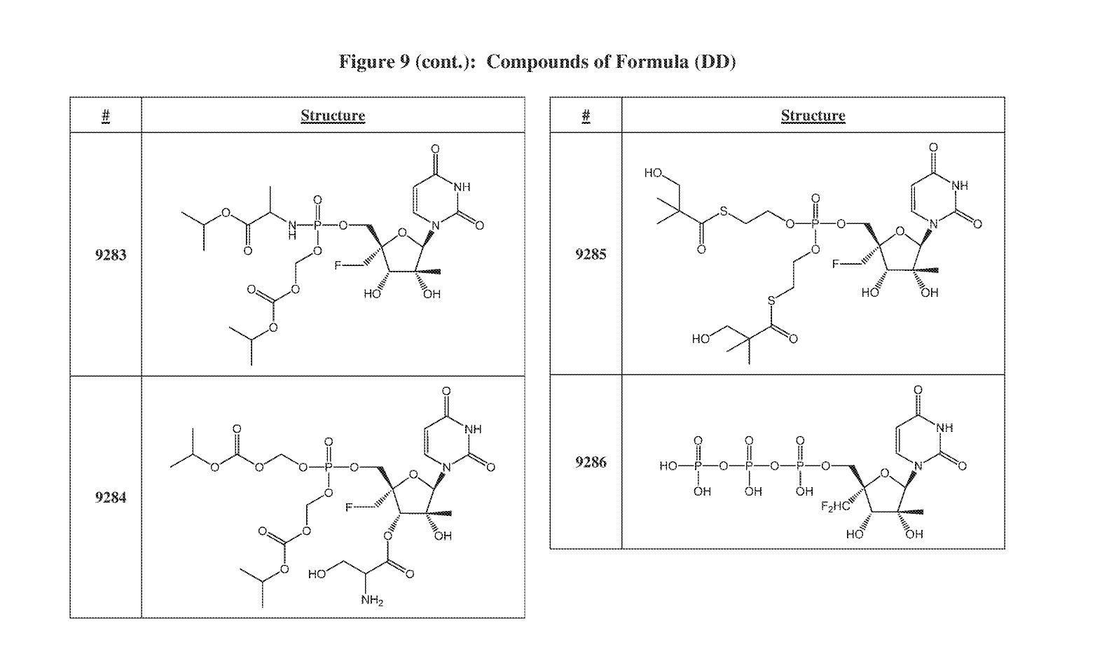

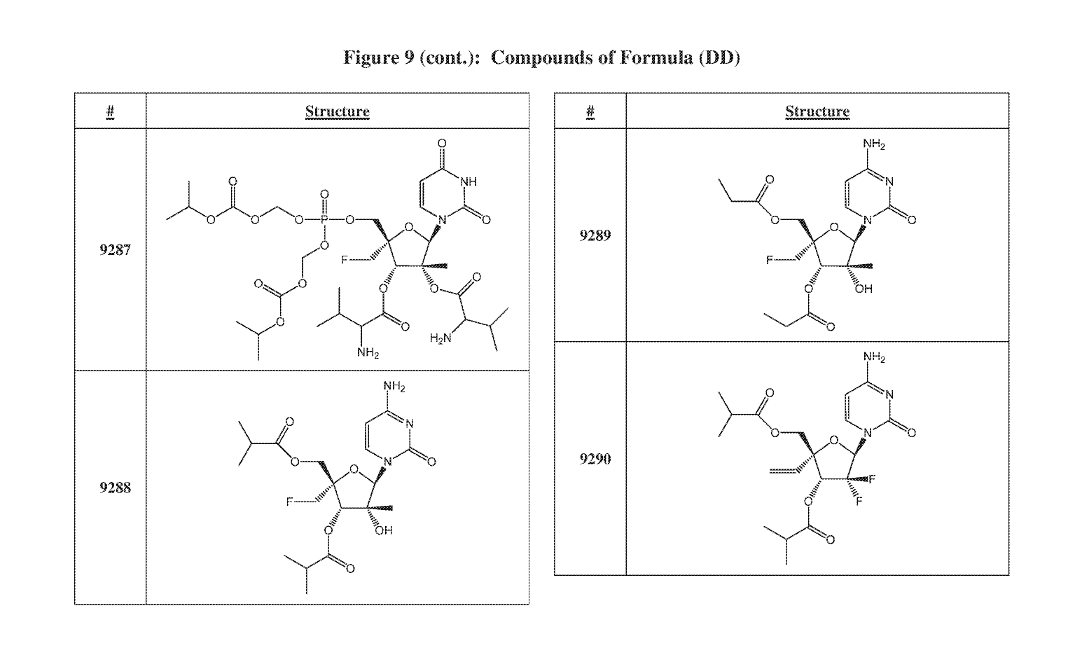

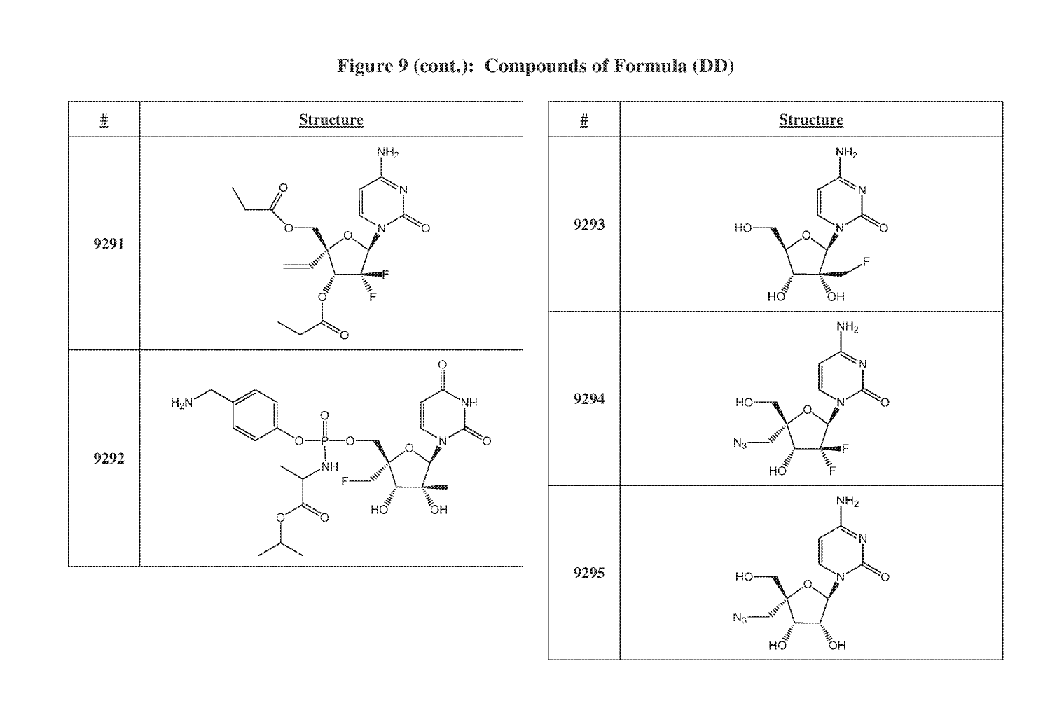

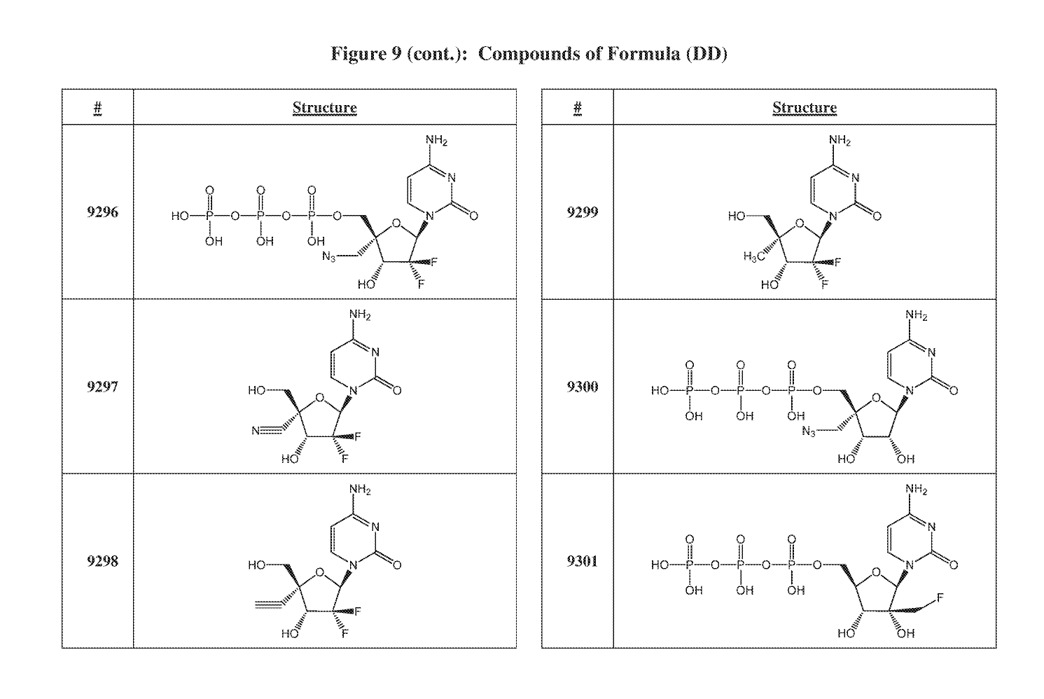

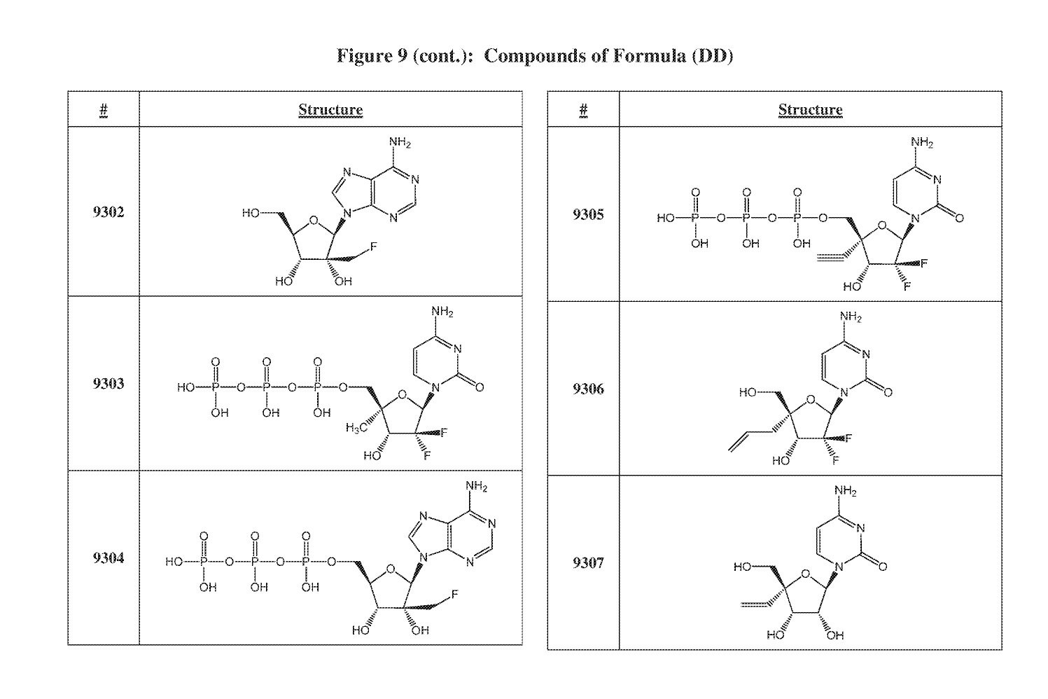

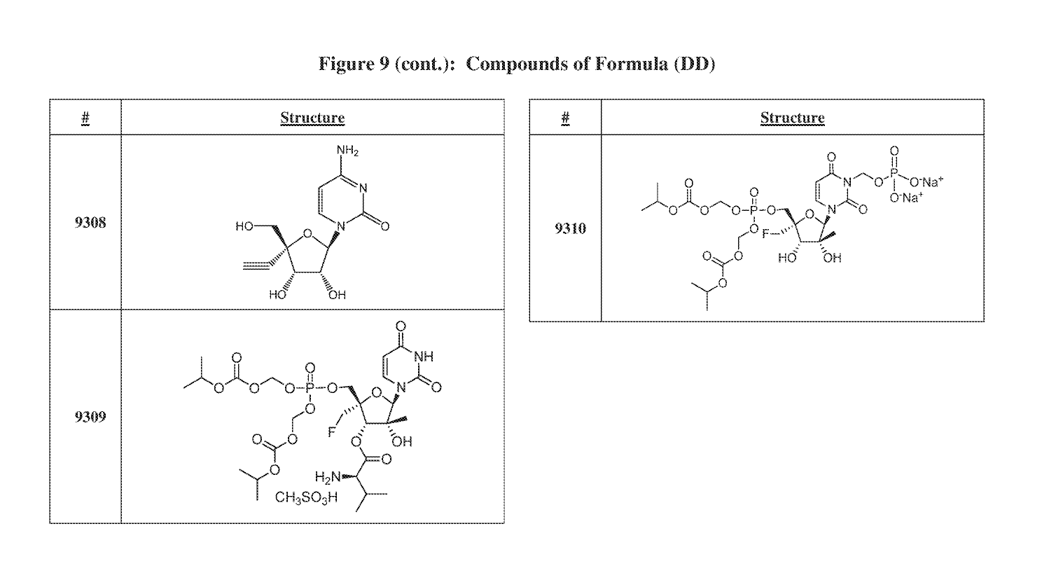

R^{25A}, R^{26A}, R^{29A}, R^{8B} and R^{9B} can be independently selected from hydrogen, an optionally substituted C₁₋₂₄ alkyl and an optionally substituted aryl; R^{27A1} and R^{27A2} can be independently selected from —C≡N, an optionally substituted C₂₋₈ organylcarbonyl, an optionally substituted C₂₋₈ alkoxycarbonyl and an optionally substituted C₂₋₈ organylaminocarbonyl; R^{28A} can be selected from hydrogen, an optionally substituted C₁₋₂₄ alkyl, an optionally substituted C₂₋₂₄ alkenyl, an optionally substituted C₂₋₂₄ alkynyl, an optionally substituted C₃₋₆ cycloalkyl and an optionally substituted C₃₋₆ cycloalkenyl; R^{30A} and R^{31A} can be independently selected from hydrogen, an optionally substituted C₁₋₂₄ alkyl, an optionally substituted C₂₋₂₄ alkenyl, an optionally substituted C₂₋₂₄ alkynyl, an optionally substituted C₃₋₆ cycloalkyl, an optionally substituted C₃₋₆ cycloalkenyl and an optionally substituted aryl (C₁₋₄ alkyl); R″^{A} and each R″^{B} can be independently an optionally substituted C₁₋₂₄ alkyl; each R″^{B1} and each R″^{B2} can be independently hydrogen or an optionally substituted C₁₋₆ alkyl; m, v and w can be independently 0 or 1; p and q can be independently 1, 2 or 3; r and s can be independently 0, 1, 2 or 3; t can be 1 or 2; u and y can be independently 3, 4 or 5; and Z^{1A}, Z^{2A}, Z^{4A}, Z^{1B} and Z^{2B} can be independently oxygen (O) or sulfur (S). In this paragraph, the asterisks indicate the points of attachment of the moieties. A non-limiting list of example compounds of Formula (DD) includes the compound numbered 9000-9310 in FIG. 9.

In some embodiments, a compound of Formulae (I) and/or (II), or a pharmaceutically acceptable salt of any of the foregoing, or a pharmaceutical composition that includes a compound of Formulae (I) and/or (II), or a pharmaceutically acceptable salt of any of the foregoing, can be used in combination with a compound of Formula (EE), or a pharmaceutically acceptable salt thereof, or a pharmaceutical composition that includes a compound of Formula (EE), or a pharmaceutically acceptable salt thereof (see, PCT Publication No. WO 2014/100505 published Jun. 26, 2014, the contents of which are incorporated by reference in its entirety):

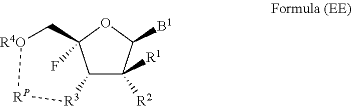

wherein: B¹ can be selected from an optionally substituted

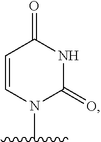 [SMILES: *n1ccc(=O)[nH]c1=O]

an optionally substituted

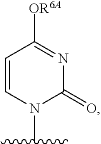

an optionally substituted

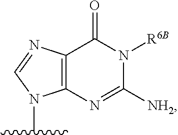

an optionally substituted

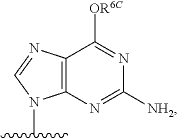

an optionally substituted

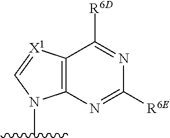

and an optionally substituted

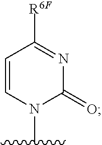

R¹ can be selected from an optionally substituted C₁₋₆ alkyl, an optionally substituted C₂₋₆ alkenyl, an optionally substituted C₂₋₆ alkenyl and an optionally substituted C₃₋₆ cycloalkyl; each -------- can be absent or a single bond, provided that both -------- are each absent or both -------- are each a single bond; when both -------- are each a single bond, then R² can be halo, N₃, —OR^{7A} or —N(R^{7B}R^{7C}); R⁴ can be absent; R³ can be oxygen (O); and R^{p} can be

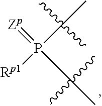

wherein Z^{p} can be oxygen (O) sulfur (S) and R^{p1} can be selected from O⁻, OH, an —O-optionally substituted C₁₋₆ alkyl.

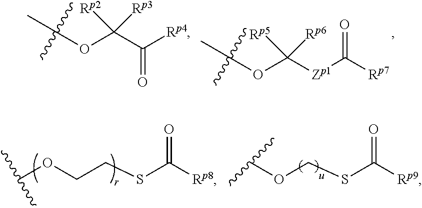

an optionally substituted N-linked amino acid and an optionally substituted N-linked amino acid ester derivative; when both -------- are each absent, then R^{p} can be absent; R² can be halo, N₃, —OR^{7A} or —N(R^{7B}R^{7C}); R³ can be —OH or —OC(═O)R⁸; or R² R³ can be each an oxygen atom which are linked together by a carbonyl group; and R⁴ can be hydrogen or

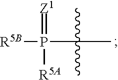

can be selected from O⁻, OH, an optionally substituted N-linked amino acid, an optionally substituted N-linked amino acid ester derivative,

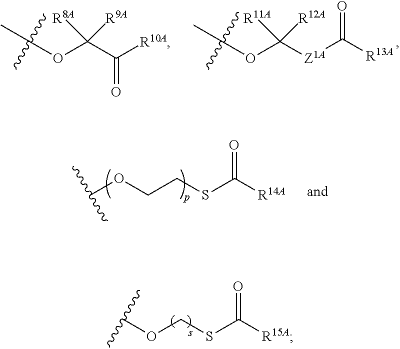

R^{5B} can be selected from O⁻, OH, an —O-optionally substituted aryl, an —O-optionally substituted heteroaryl, an —O-optionally substituted heterocyclyl, an optionally substituted N-linked amino acid, an optionally substituted N-linked amino acid ester derivative,

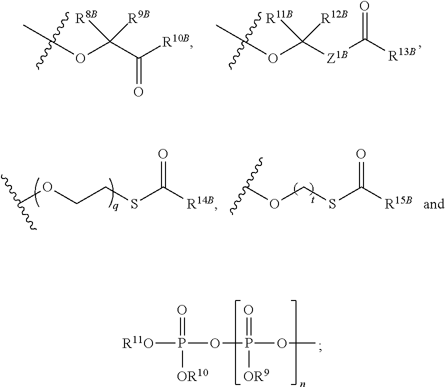

R^{6A} can be an optionally substituted C₁₋₆ alkyl or an optionally substituted C₃₋₆ cycloalkyl; R^{6B} and R^{6C} can be independently selected from hydrogen, an unsubstituted C₁₋₆ alkyl, an unsubstituted C₃₋₆ alkenyl, an unsubstituted C₃₋₆ alkynyl and an unsubstituted C₃₋₆ cycloalkyl; R^{6D} can be NHR^{6G}; R^{6E} can be hydrogen, halogen or NHR^{6H}; R^{6F} can be NHR^{6L}; R^{6G} can be selected from hydrogen, an optionally substituted C₁₋₆ alkyl, an optionally substituted C₃₋₆ alkenyl, an optionally substituted C₃₋₆ cycloalkyl, —C(═O)R^{A1} and —C(═O)OR^{A2}; R^{6H} can be selected from hydrogen, an optionally substituted C₁₋₆ alkyl, an optionally substituted C₃₋₆ alkenyl, an optionally substituted C₃₋₆ cycloalkyl, —C(═O)R^{A3} and —C(═O)OR^{A4}; R^{6I} can be selected from hydrogen, an optionally substituted C₁₋₆ alkyl, an optionally substituted C₃₋₆ alkenyl, an optionally substituted C₃₋₆ cycloalkyl, —C(═O)R^{A5} and —C(═O)OR^{A6}; X¹ can be N (nitrogen) or —CR^{6J}, R^{6J} can be selected from hydrogen, halogen, an optionally substituted C₁₋₆ alkyl, an optionally substituted C₂₋₆ alkenyl and an optionally substituted C₂₋₆ alkynyl; R^{A1}, R^{A2}, R^{A3}, R^{A4}, R^{A5} and R^{A6} can be independently selected from C₁₋₆ alkyl, C₂₋₆ alkenyl, C₂₋₆ alkynyl, C₃₋₆ cycloalkyl, C₃₋₆ cycloalkenyl, C₆₋₁₀ aryl, heteroaryl, heterocyclyl, aryl (C₁₋₆ alkyl), heteroaryl (C₁₋₆ alkyl) and heterocyclyl (C₁₋₆ alkyl); R^{7A} can be hydrogen or —C(═O)R¹²; R^{7B} and R^{7C} can be independently hydrogen or an optionally substituted C₁₋₆ alkyl; R⁸ and R¹² can be independently an optionally substituted C₁₋₆ alkyl or an optionally substituted C₃₋₆ cycloalkyl; R⁹, R¹⁰ and R¹¹ can be independently absent or hydrogen; R^{8A}, R^{9A}, R^{11A}, R^{12A}, R^{8B}, R^{9B}, R^{11B}, R^{12B}, R^{p2}, R^{p3}, R^{p5} and R^{p6} can be independently selected from hydrogen, an optionally substituted C₁₋₂₄ alkyl and an optionally substituted aryl; R^{10A}, R^{10B}, R^{13A}, R^{13B}, R^{p4} and R^{p7} can be independently selected from hydrogen, an optionally substituted C₁₋₂₄ alkyl, an optionally substituted aryl, an optionally substituted —O—C₁₋₂₄ alkyl, an optionally substituted —O-aryl, an optionally substituted —O-heteroaryl and an optionally substituted —O-monocyclic heterocyclyl; R^{14A}, R^{14B}, R^{15A}, R^{15B}, R^{p8} and R^{p9} can be independently selected from hydrogen, an optionally substituted C₁₋₂₄ alkyl and an optionally substituted aryl; n can be 0 or 1; p, q and r can be independently 1 or 2; s, t and u can be independently 3, 4 or 5; Z¹, Z^{1A}, Z^{1B} and Z^{p1} can be independently O (oxygen) or S (sulfur); and provided that when R⁴ is

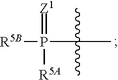

and R^{5A} is O⁻ or OH, then R^{5B} is O⁻, OH,

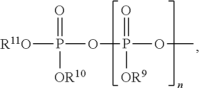

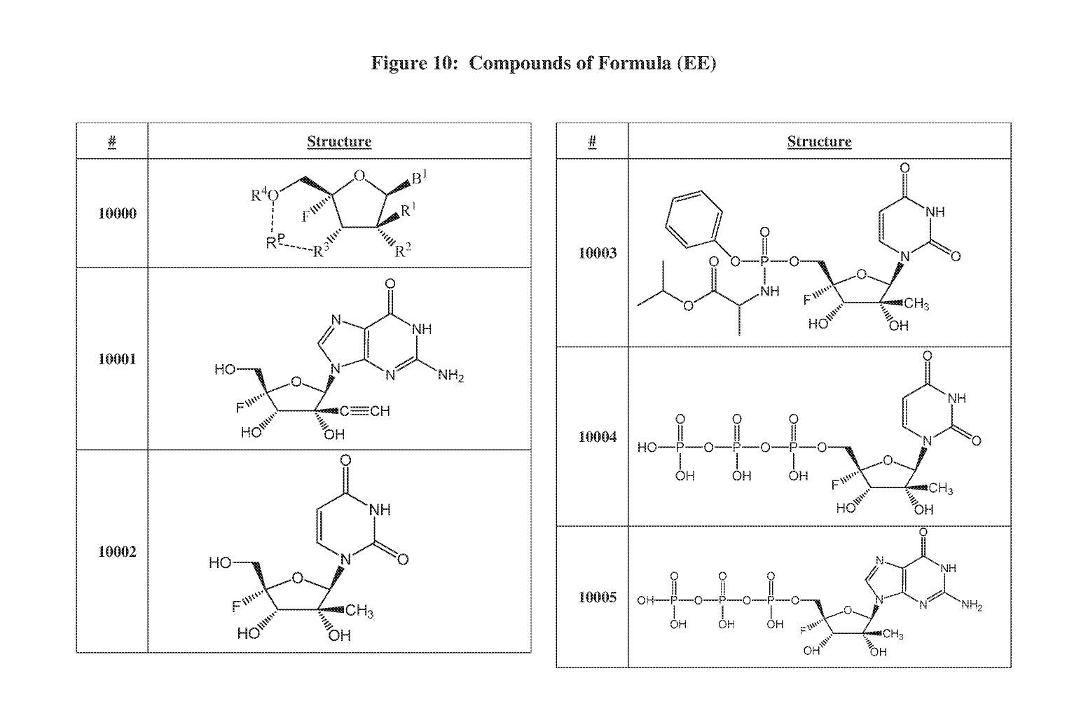

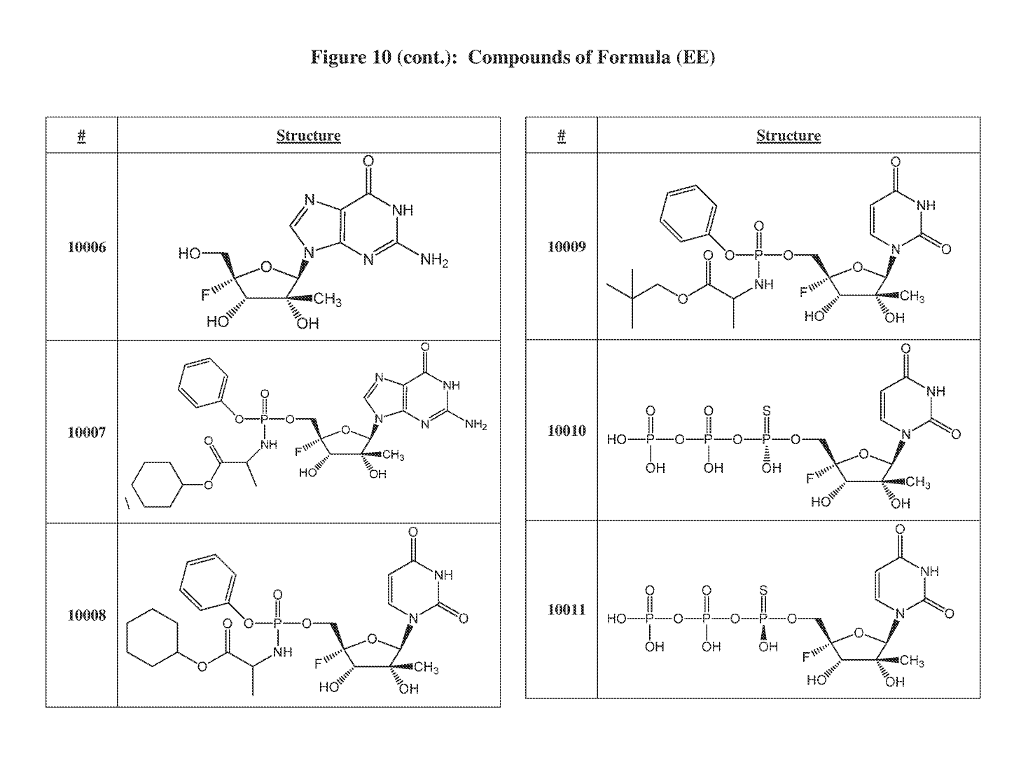

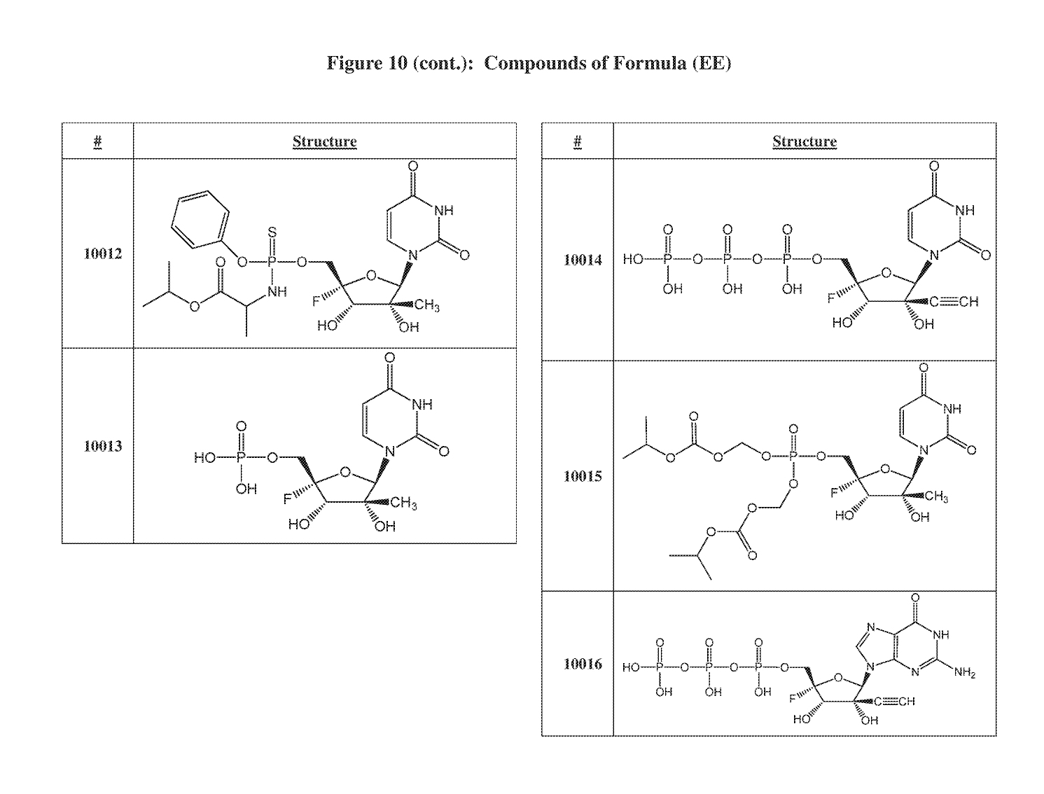

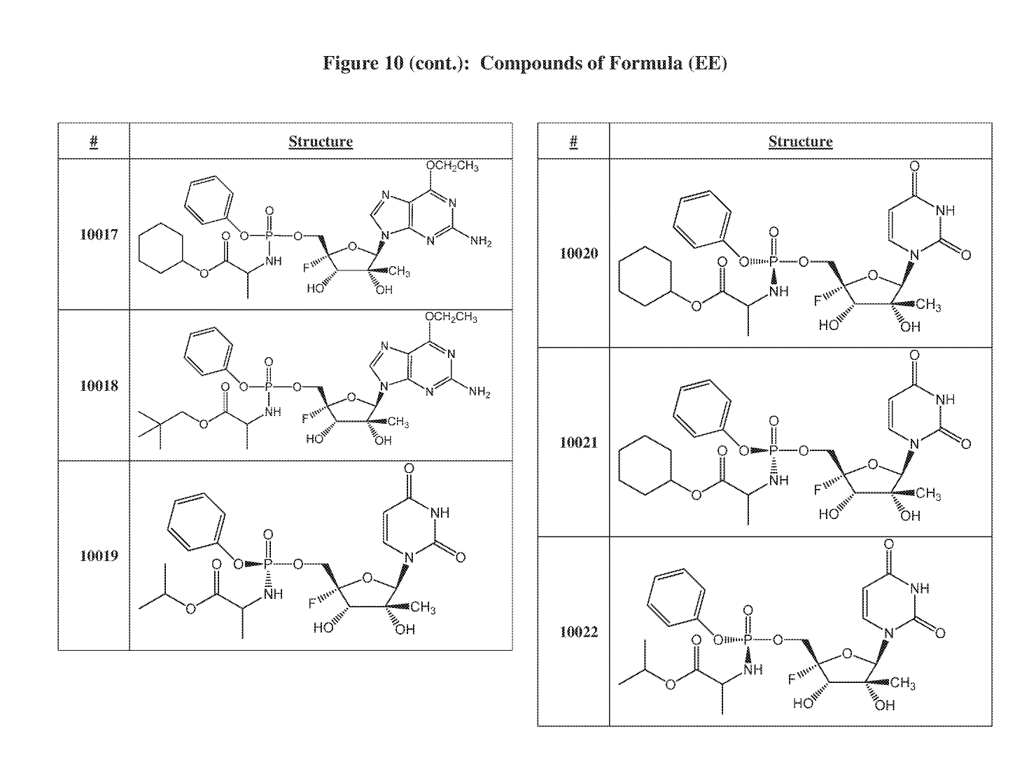

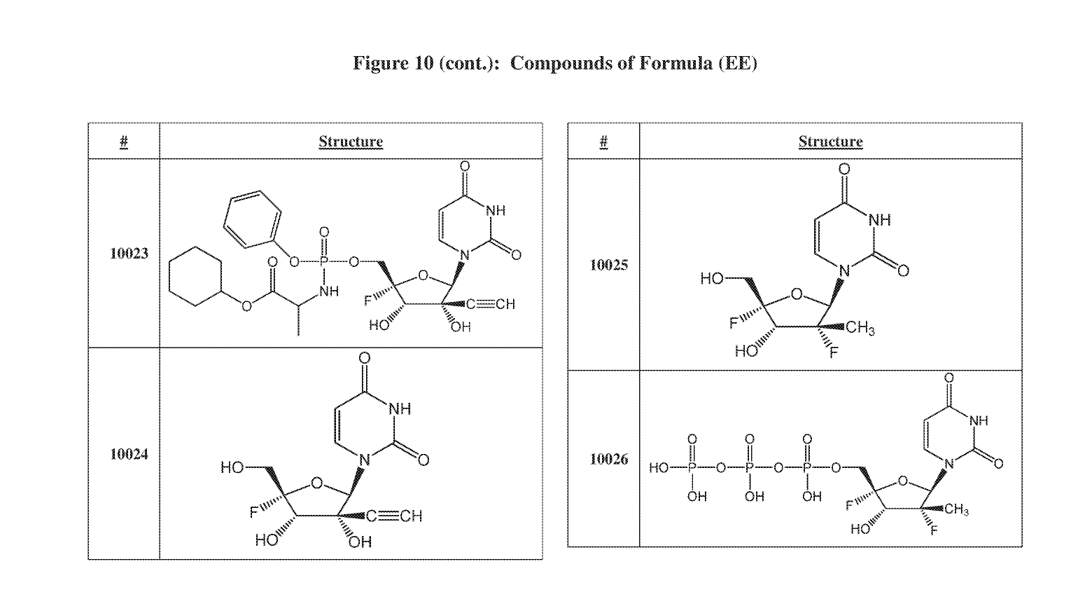

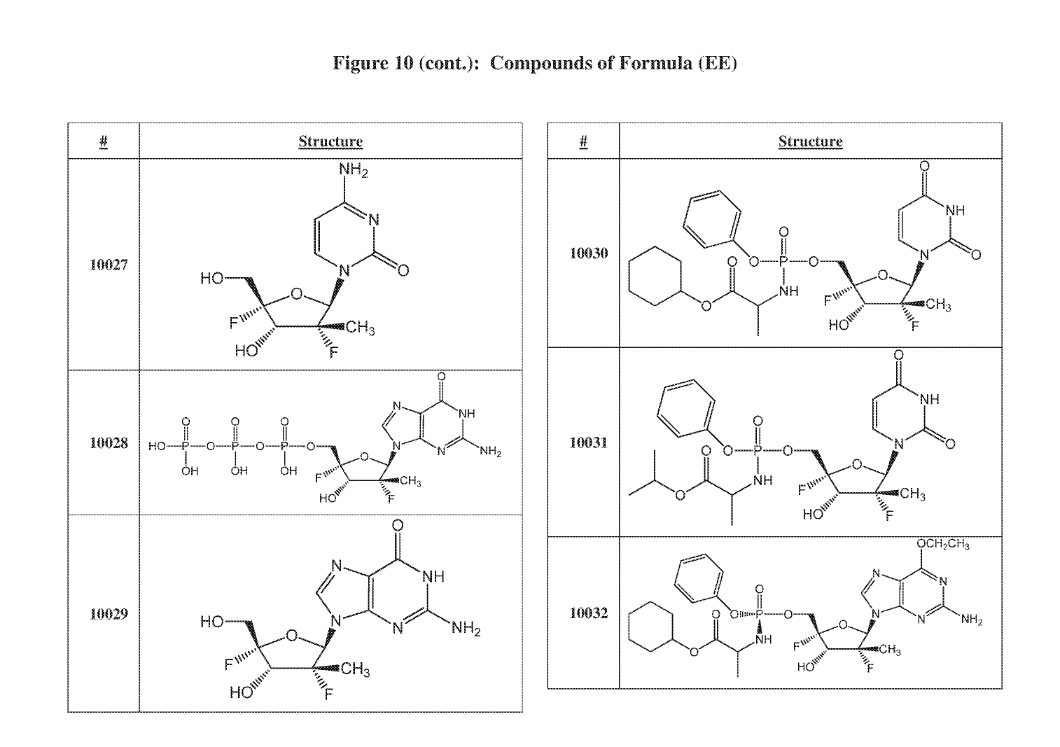

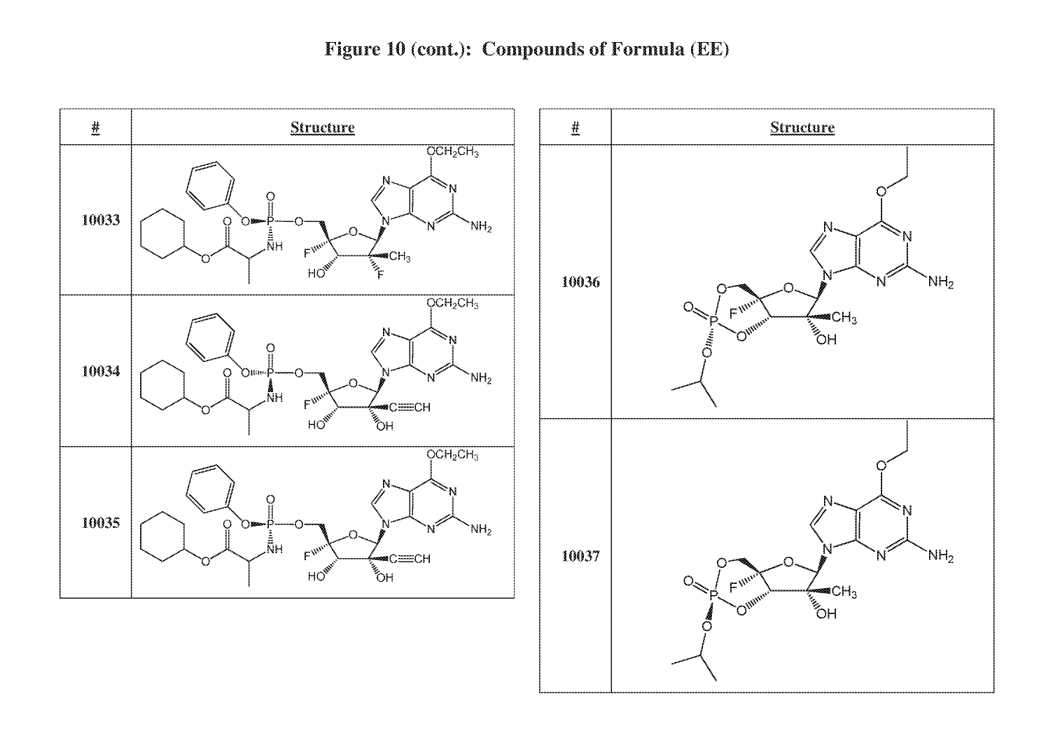

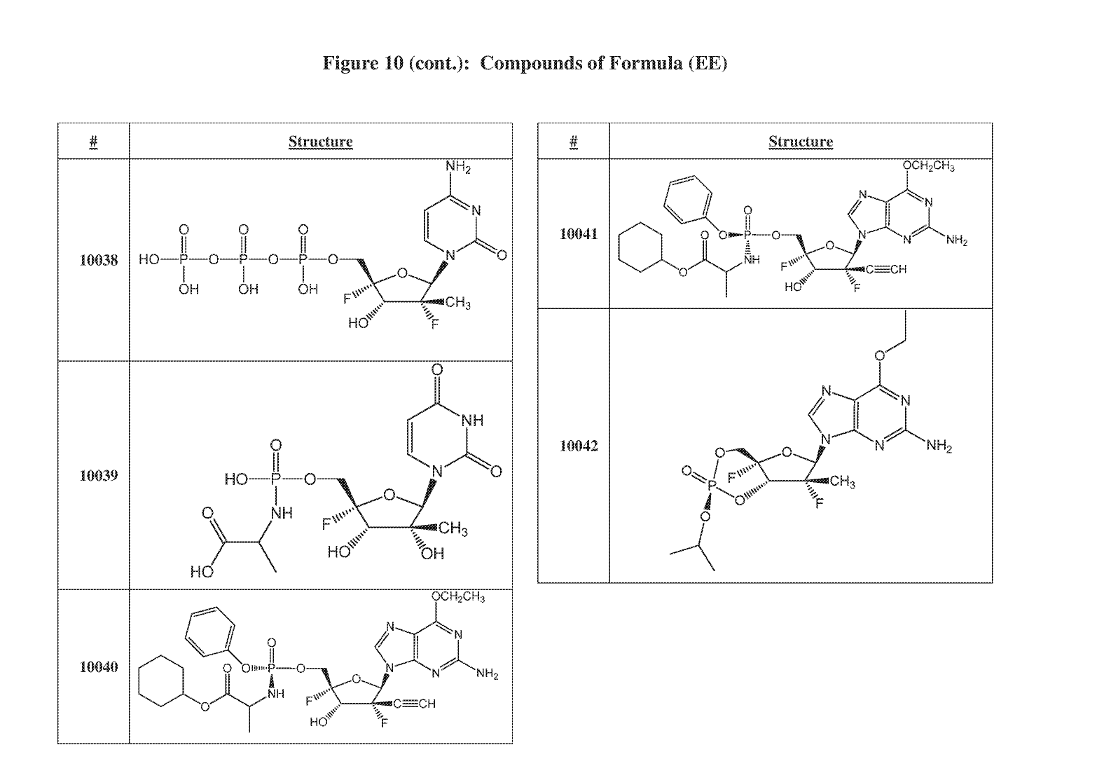

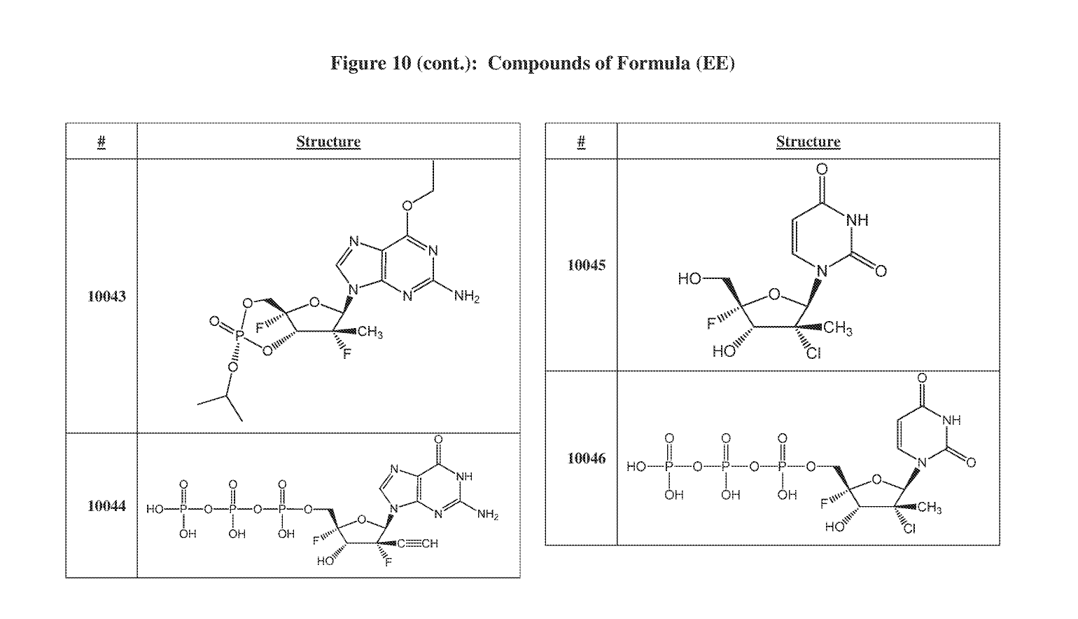

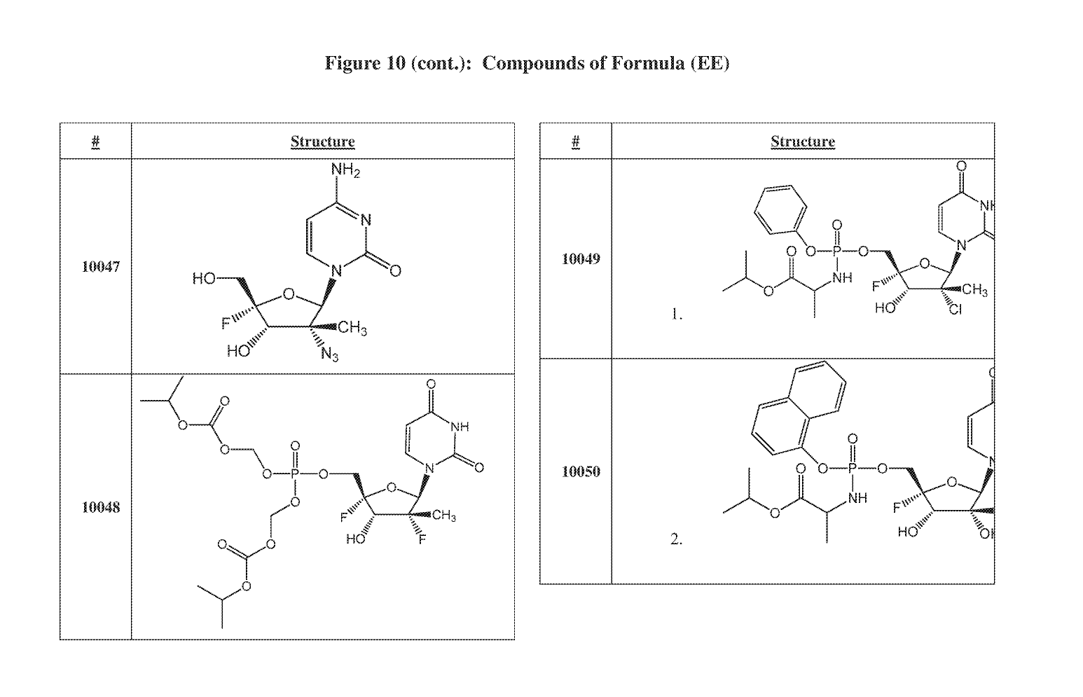

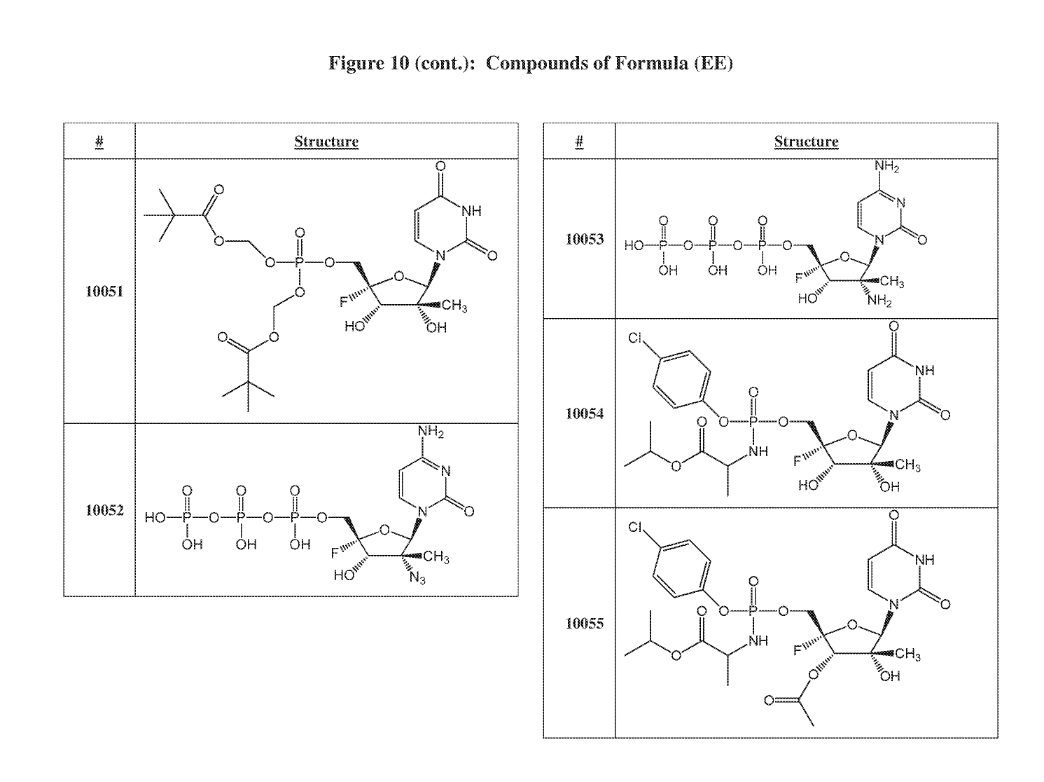

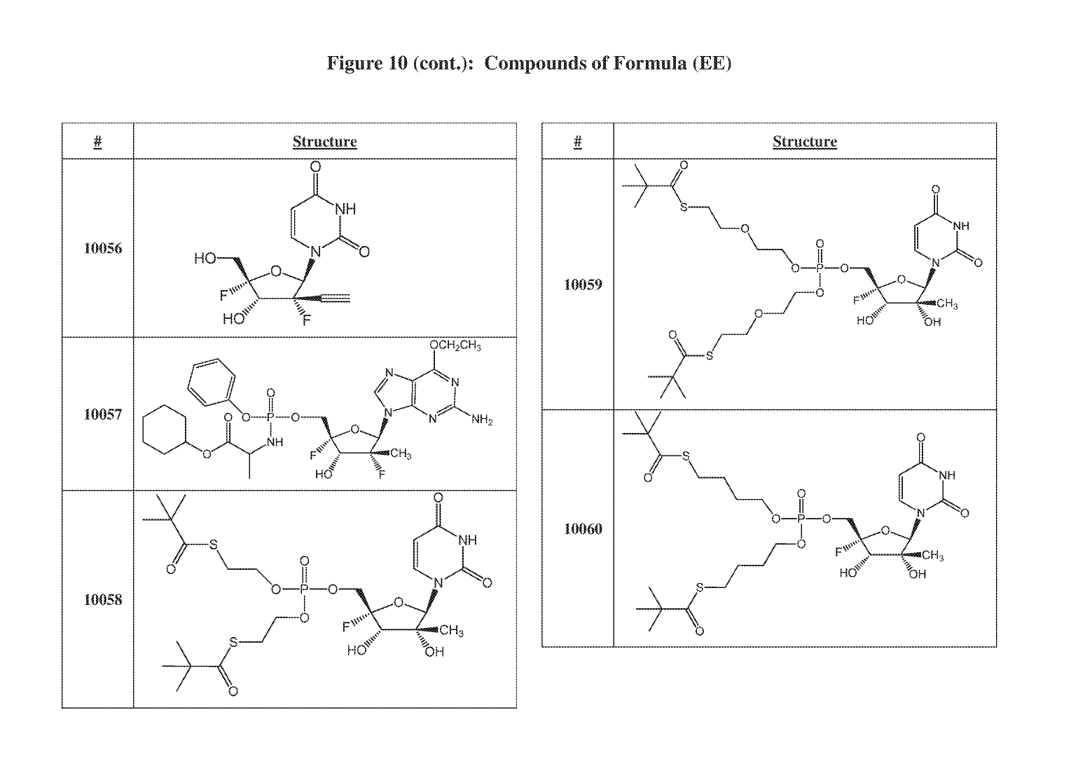

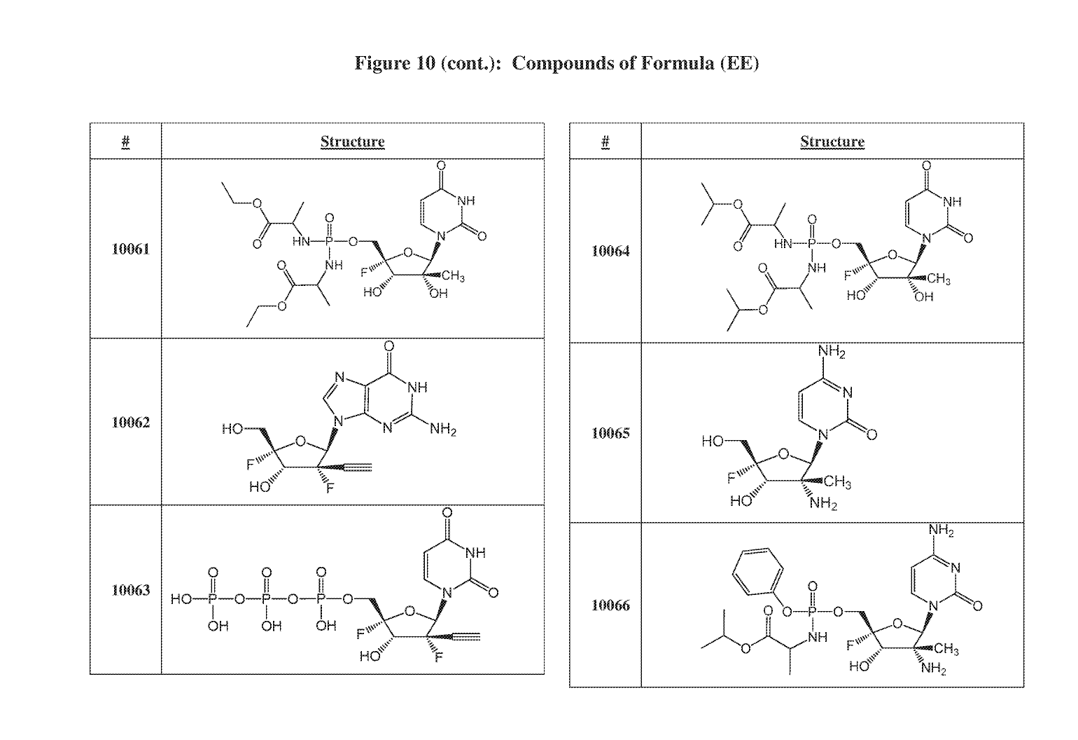

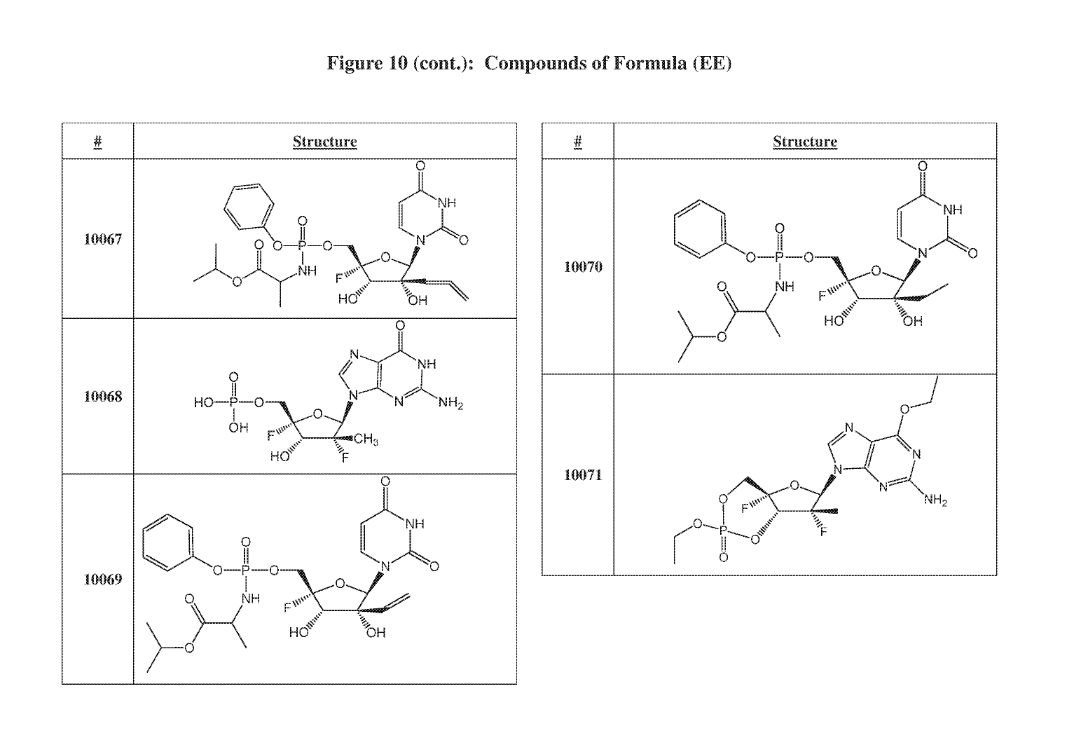

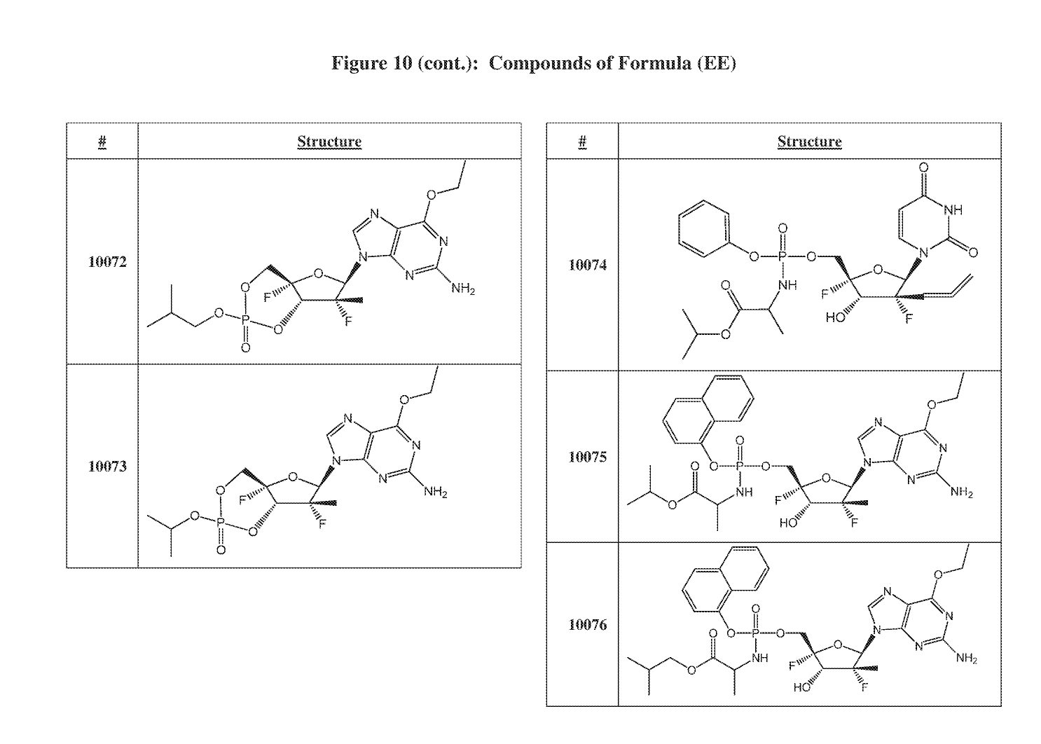

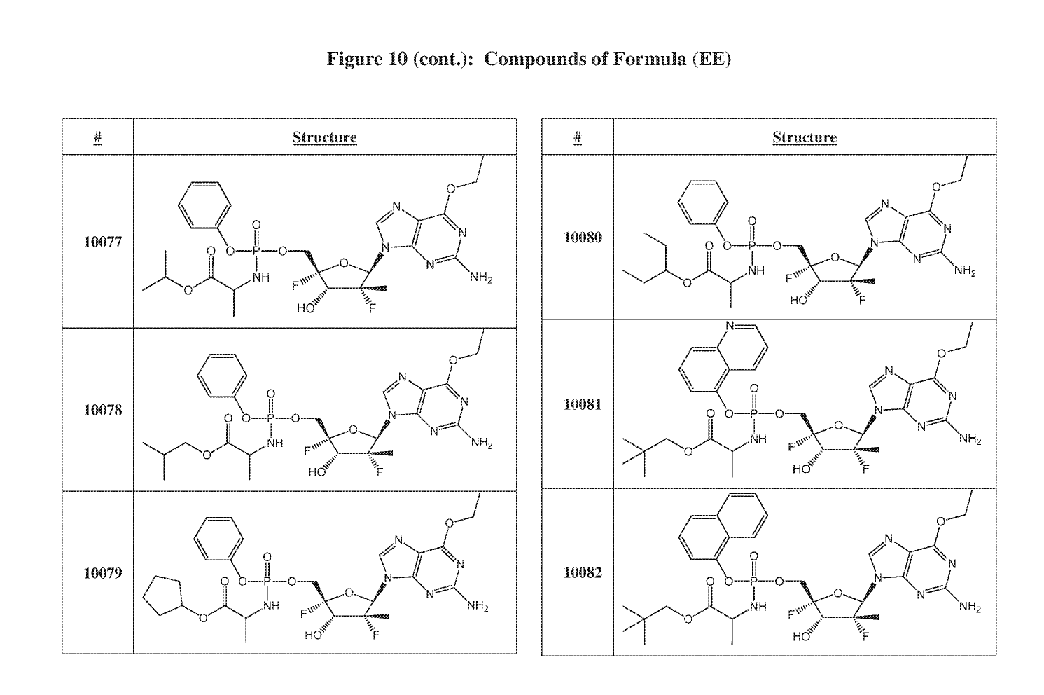

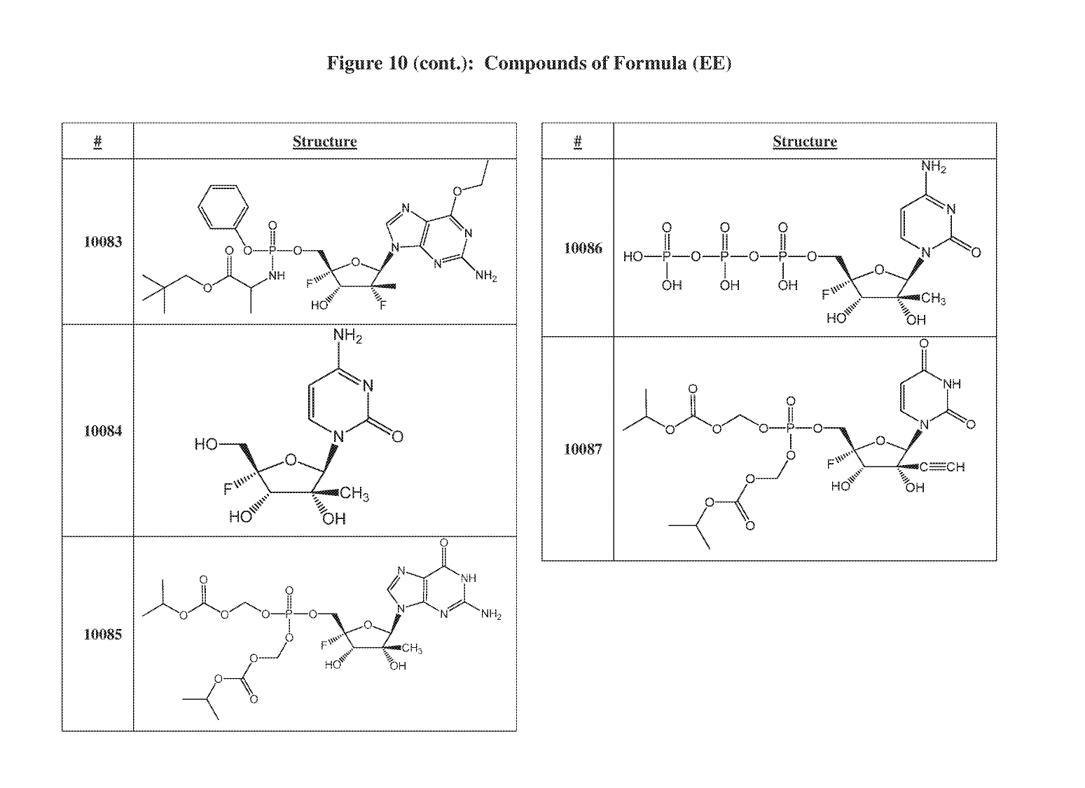

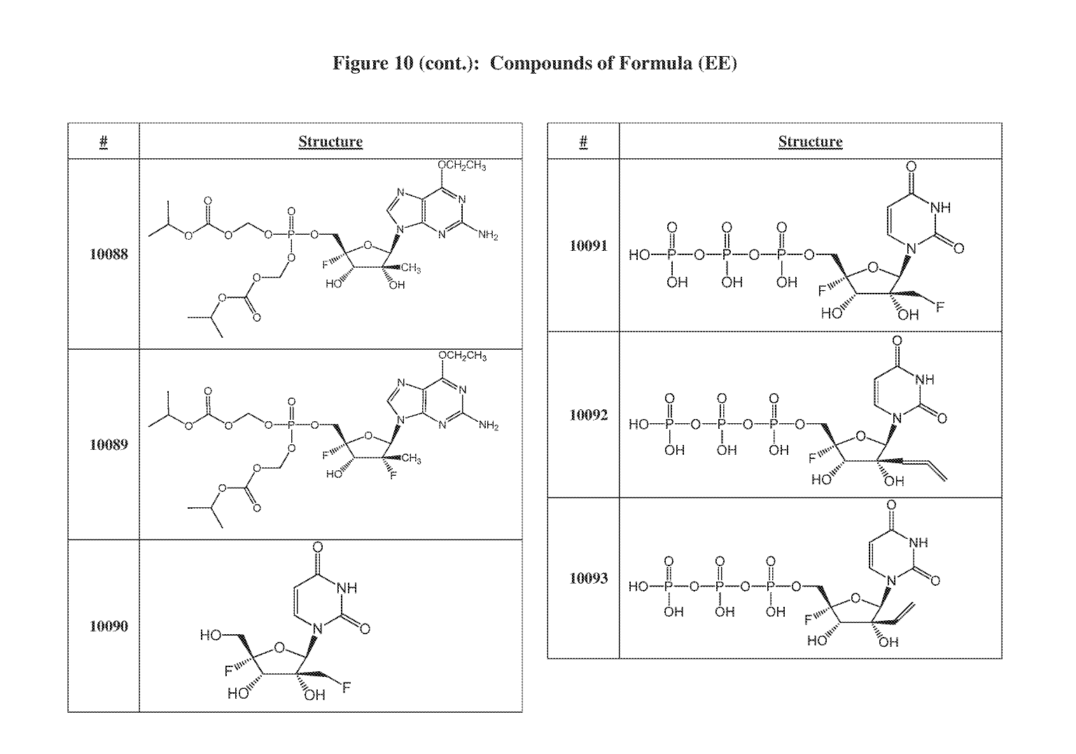

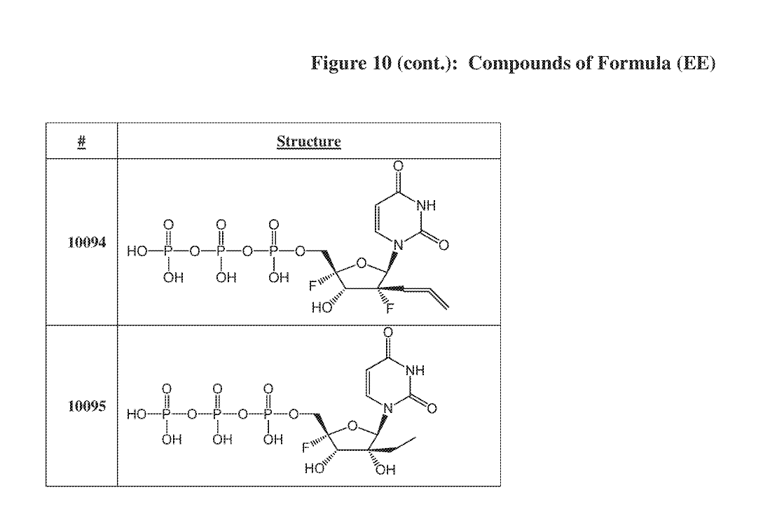

an optionally substituted N-linked amino acid or an optionally substituted N-linked amino acid ester derivative. A non-limiting list of example compounds of Formula (EE) includes the compound numbered 10000-10095 in FIG. 10.

In some embodiments, a compound of Formulae (I) and/or (II), or a pharmaceutically acceptable salt of any of the foregoing, or a pharmaceutical composition that includes a compound of Formulae (I) and/or (II), or a pharmaceutically acceptable salt of any of the foregoing, can be used in combination with a compound of Formula (FF), or a pharmaceutically acceptable salt thereof, or a pharmaceutical composition that includes a compound of Formula (FF), or a pharmaceutically acceptable salt thereof (see, PCT Publication No. WO 2014/100498 published Jun. 26, 2014, the contents of which are incorporated by reference in its entirety):

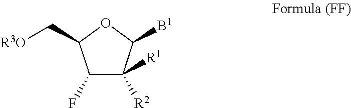

wherein: B¹ can be an optionally substituted

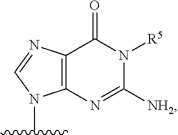

an optionally substituted

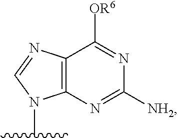

or an optionally substituted

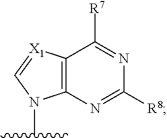

R¹ can be selected from an unsubstituted C₁₋₆ alkyl, an unsubstituted C₂₋₆ alkenyl, an unsubstituted C₂₋₆ alkynyl, an unsubstituted C₃₋₆ cycloalkyl and an unsubstituted C₁₋₆ haloalkyl; R² can be halo, —OR^{9A} or —N(R^{9B}R^{9C}); R³ can be hydrogen or

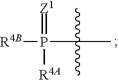

R^{4A} can be selected from O—, OH, an optionally substituted N-linked amino acid and an optionally substituted N-linked amino acid ester derivative; R^{4B} can be selected from O⁻, OH, an —O-optionally substituted aryl, an —O-optionally substituted heteroaryl, an —O-optionally substituted heterocyclyl, an optionally substituted N-linked amino acid, an optionally substituted N-linked amino acid ester derivative and

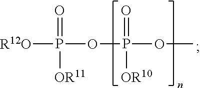

R⁵ and R⁶ can be independently selected from hydrogen, an unsubstituted C₁₋₆ alkyl, an unsubstituted C₃₋₆ alkenyl, an unsubstituted C₃₋₆ alkynyl and an unsubstituted C₃₋₆ cycloalkyl; R⁷ can be NHR¹³; R⁸ can be NHR¹⁴; R^{9A} can be hydrogen or —C(═O)R¹⁵; R^{9B} and R^{9C} can be independently hydrogen or an optionally substituted C₁₋₆ alkyl; R¹⁰, R¹¹ and R¹² can be independently absent or hydrogen; R¹³ can be selected from hydrogen, an optionally substituted C₁₋₆ alkyl, an optionally substituted C₃₋₆ alkenyl, an optionally substituted cycloalkyl, —C(═O)R^{A1} and —C(═O)OR^{A2}; R¹⁴ can be selected from hydrogen, an optionally substituted C₁₋₆ alkyl, an optionally substituted C₃₋₆ alkenyl, an optionally substituted C₃₋₆ cycloalkyl, —C(═O)R^{A3} and —C(═O)OR^{A4}; R¹⁵ can be an optionally substituted C₁₋₆ alkyl or an optionally substituted C₃₋₆ cycloalkyl; X¹ can be N or —CR¹⁶; R¹⁶ can be selected from hydrogen, halogen, an optionally substituted C₁₋₆ alkyl, an optionally substituted C₂₋₆ alkenyl and an optionally substituted C₂₋₆ alkynyl; R^{A1}, R^{A2}, R^{A3} and R^{A4} can be independently selected from C₁₋₆ alkyl, C₂₋₆ alkenyl, C₂₋₆ alkynyl, C₃₋₆ cycloalkyl, C₃₋₁₀ cycloalkenyl, C₆₋₁₀ aryl, heteroaryl, heteroalicyclyl, aryl (C₁₋₆ alkyl), heteroaryl (C₁₋₆ alkyl) and heteroalicyclyl (C₁₋₆ alkyl); n can be 0 or 1; Z¹ can be O or S; and provided that when R³ is

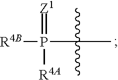

and R^{4A} is O⁻ or OH, then R^{4B} is O⁻, OH or

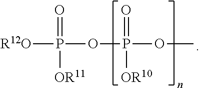

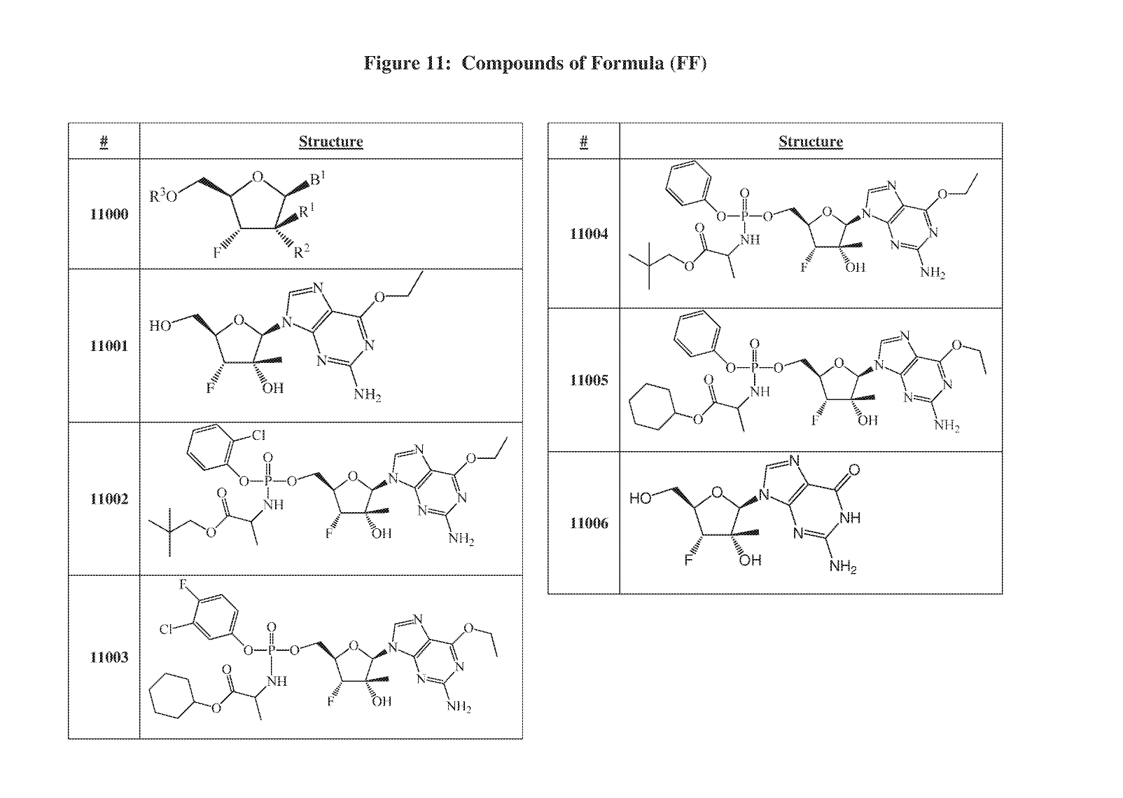

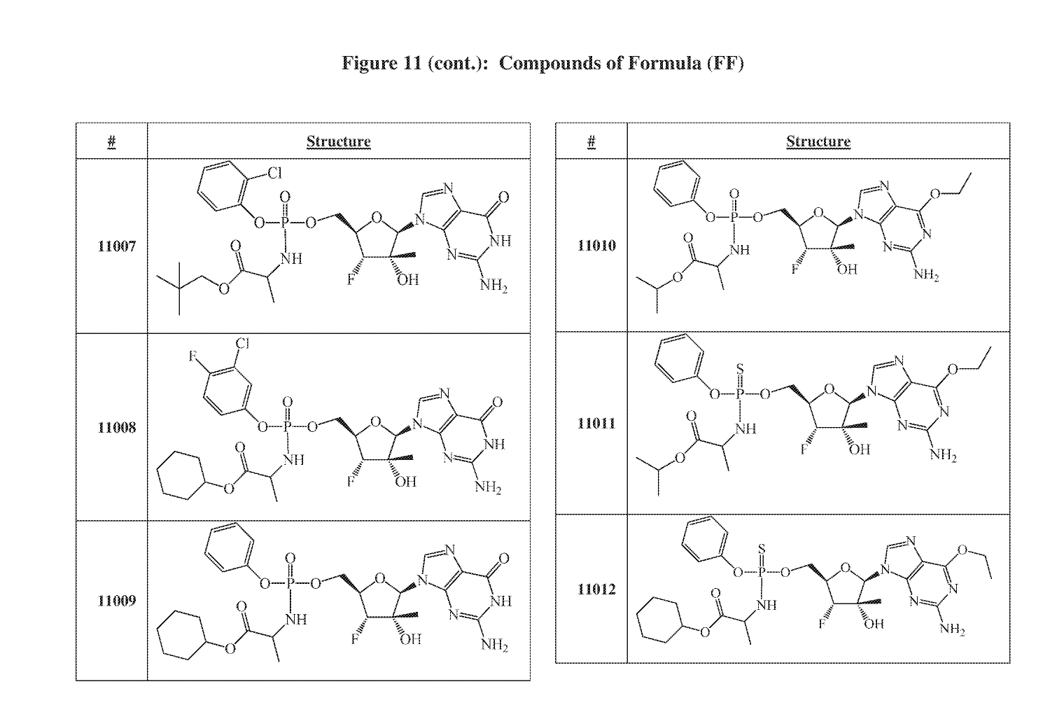

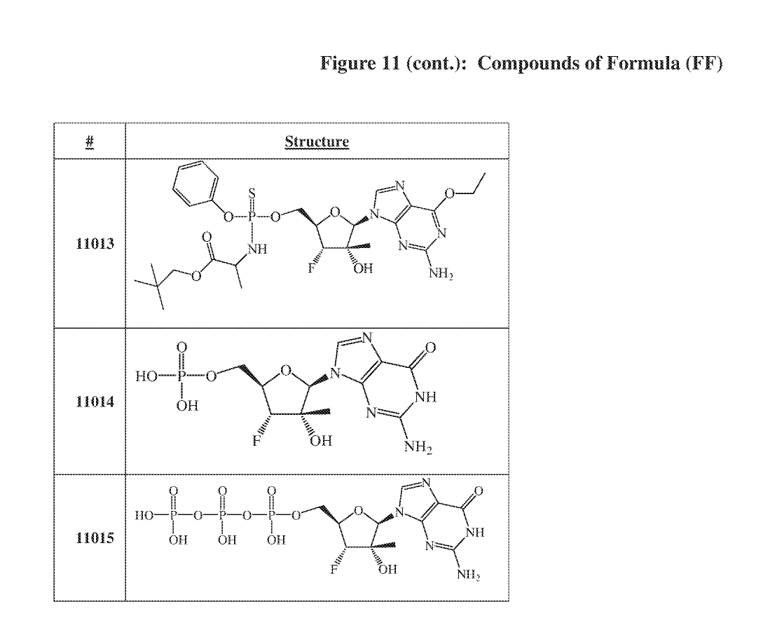

A non-limiting list of example compounds of Formula (FF) includes the compound numbered 11000-11015 in FIG. 11.

Some embodiments described herein relate to a method of ameliorating or treating a picornavirus and/or a Flaviviridae viral infection that can include contacting a cell infected with the virus with an effective amount of a compound of Formulae (I) and/or (II), or a pharmaceutically acceptable salt of any of the foregoing, in combination with one or more agents selected from an interferon, ribavirin, a compound of Formula (AA), a compound of Formula (BB), a compound of Formula (CC), a compound of Formula (DD), a compound of Formula (EE), and a compound of Formula (FF), or a pharmaceutically acceptable salt of any of the aforementioned compounds. Some embodiments described herein relate to a method of ameliorating or treating a HCV infection that can include contacting a cell infected with the HCV infection with an effective amount of a compound of Formulae (I) and/or (II), or a pharmaceutically acceptable salt of any of the foregoing, in combination with one or more agents selected from an interferon, ribavirin, a HCV protease inhibitor, a HCV polymerase inhibitor, a NS5A inhibitor, an antiviral compound, a compound of Formula (AA), a compound of Formula (BB), a compound of Formula (CC), a compound of Formula (DD), a compound of Formula (EE) and a compound of Formula (FF), or a pharmaceutically acceptable salt of any of the aforementioned compounds.

Some embodiments described herein relate to a method of ameliorating or treating a picornavirus and/or a Flaviviridae viral infection that can include administering to a subject suffering from the viral infection an effective amount of a compound of Formulae (I) and/or (II), or a pharmaceutically acceptable salt of any of the foregoing, in combination with one or more agents selected from an interferon, ribavirin, a compound of Formula (AA), a compound of Formula (BB), a compound of Formula (CC), a compound of Formula (DD), a compound of Formula (EE) and a compound of Formula (FF), or a pharmaceutically acceptable salt of any of the aforementioned compounds. Some embodiments described herein relate to a method of ameliorating or treating a HCV infection that can include administering to a subject suffering from the HCV infection an effective amount of a compound of Formulae (I) and/or (II), or a pharmaceutically acceptable salt of any of the foregoing, in combination with one or more agents selected from an interferon, ribavirin, a HCV protease inhibitor, a HCV polymerase inhibitor, a NS5A inhibitor, an antiviral compound, a compound of Formula (AA), a compound of Formula (BB), a compound of Formula (CC), a compound of Formula (DD), a compound of Formula (EE) and a compound of Formula (FF), or a pharmaceutically acceptable salt of any of the aforementioned compounds.

Some embodiments described herein relate to a method of inhibiting the replication of a Picornavirus and/or a Flaviviridae virus that can include contacting a cell infected with the virus with an effective amount of a compound of Formulae (I) and/or (II), or a pharmaceutically acceptable salt of any of the foregoing, in combination with one or more agents selected from an interferon, ribavirin, a compound of Formula (AA), a compound of Formula (BB), a compound of Formula (CC), a compound of Formula (DD), a compound of Formula (EE) and a compound of Formula (FF), or a pharmaceutically acceptable salt of any of the aforementioned compounds. Some embodiments described herein relate to a method of inhibiting the replication of a hepatitis C virus that can include contacting a cell infected with the hepatitis C virus with an effective amount of a compound of Formulae (I) and/or (II), or a pharmaceutically acceptable salt of any of the foregoing, in combination with one or more agents selected from an interferon, ribavirin, a HCV protease inhibitor, a HCV polymerase inhibitor, a NS5A inhibitor, an antiviral compound, a compound of Formula (AA), a compound of Formula (BB), a compound of Formula (CC), a compound of Formula (DD), a compound of Formula (FE) and a compound of Formula (FF), or a pharmaceutically acceptable salt of any of the aforementioned compounds.

Some embodiments described herein relate to a method of inhibiting the replication of a Picornaviridae and/or a Flaviviridae virus that can include administering to a subject infected with the virus an effective amount of a compound of Formulae (I) and/or (II), or a pharmaceutically acceptable salt of any of the foregoing, in combination with one or more agents selected from an interferon, ribavirin, a compound of Formula (AA), a compound of Formula (BB), a compound of Formula (CC), a compound of Formula (DD), a compound of Formula (EE) and a compound of Formula (FF), or a pharmaceutically acceptable salt of any of the aforementioned compounds. Some embodiments described herein relate to a method of inhibiting the replication of a hepatitis C virus that can include administering to a subject infected with the hepatitis C virus an effective amount of a compound of Formulae (I) and/or (II), or a pharmaceutically acceptable salt of any of the foregoing, in combination with one or more agents selected from an interferon, ribavirin, a HCV protease inhibitor, a HCV polymerase inhibitor, a NS5A inhibitor, an antiviral compound, a compound of Formula (AA), a compound of Formula (BB), a compound of Formula (CC), a compound of Formula (DD), a compound of Formula (EE) and a compound of Formula (FF), or a pharmaceutically acceptable salt of any of the aforementioned compounds. In some embodiments described herein, the combination of agents can be used to treat, ameliorate and/or inhibit a virus and/or a viral infection, wherein the virus can be Picornaviridae and/or Flaviviridae virus and the viral infection can be a Picornaviridae and/or Flaviviridae viral infection.

### EXAMPLES

Additional embodiments are disclosed in further detail in the following examples, which are not in any way intended to limit the scope of the claims.

### Intermediate 1

### (2R,3R,4R,5R)-5-((benzoyloxy)methyl)-3-ethynyltetrahydrofuran-2,3,4-triyltribenzoate

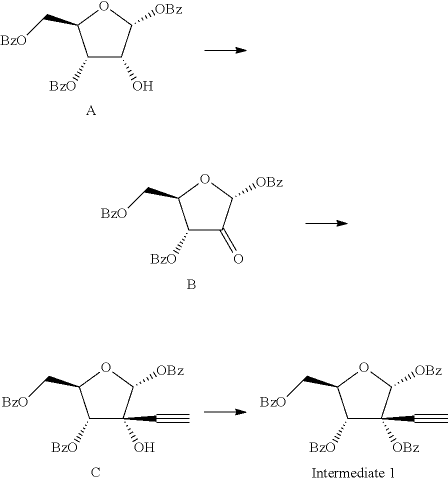

Compound B: To a solution of compound A ((2R,3R,4S,5R)-5-((benzoyloxy)methyl)-3-hydroxytetrahydrofuran-2,4-diyldibenzoate, 15 g, 32.4 mmol) in ACN (ACN, 150 mL) was added IBX (2-iodoxybenzoic acid) (18.18 g, 64.9 mmol) at room temperature (R.T.). The solution was stirred for 16 h at 80° C. and then cooled to R.T. The solid was filtered and the resulting solution was concentrated under reduced pressure to provide compound B ((2R,4R,5R)-5-((benzoyloxy)methyl)-3-oxotetrahydrofuran-2,4-diyldibenzoate, 14.1 g, 94%) as a yellow solid. MS m/z (ESI): 461 [M+H]⁺.

Compound C: To a solution of compound B (20 g, 43.4 mmol) in THF (200 mL) was added ethynylmagnesium bromide (0.5 M in THF, 348 mL) at −78° C. to −30° C. The solution was stirred for 2 h at −30° C. The reaction was quenched by the addition of sat. NH₄Cl solution (500 mL). The solution was extracted with ethyl acetate (EA, 2×500 mL). The extracts were dried over anhydrous Na₂SO₄, filtered and concentrated under reduced pressure to provide compound C ((2R,3R,4R,5R)-5-((benzoyloxy)methyl)-3-ethynyl-3-hydroxytetrahydrofuran-2,4-diyldibenzoate, 18.7 g, crude) as a brown solid. MS m/z (ESI): 509 [M+Na]⁺.

Intermediate 1: To a solution of compound C (5 g, 10.3 mmol) in DCM (50 mL) was added DHAP (2.51 g, 20.6 mmol) and triethylamine (3.12 g, 30.8 mmol). Benzoyl chloride (4.35 g, 31 mmol) was then added at 0° C. The solution was stirred for 16 h at R.T., diluted with DCM (500 mL) and washed with NaHCO₃ solution (500 mL). The solution was dried over anhydrous Na₂SO₄, filtered and concentrated under reduced pressure. The residue was applied onto a silica gel column with EA:PE (petroleum ether) (1:10-1:5) to provide Intermediate 1 ((2R,3R,4R,5R)-5-((benzoyloxy)methyl)-3-ethynyltetrahydrofuran-2,3,4-triyltribenzoate, 4.1 g, 68%) as a white solid. MS m/z (ESI): 613 [M+Na]⁺.

### Intermediate 2

### (3R,4R,5R)-5-((benzoyloxy)methyl)-3-methyltetrahydrofuran-2,3,4-triyltribenzoate

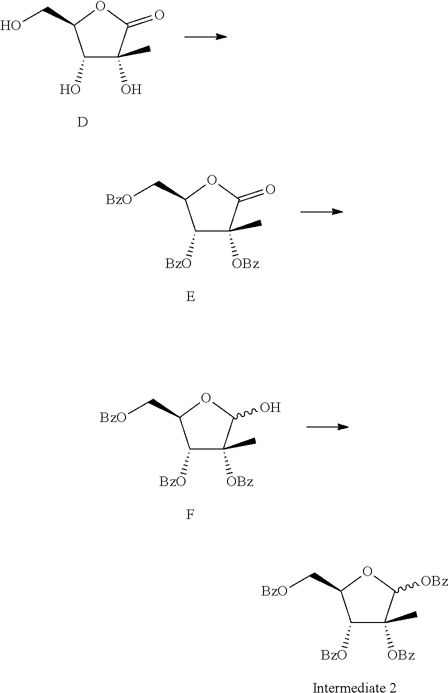

Compound E: To a solution of compound D ((3R,4R,5R)-3,4-dihydroxy-5-(hydroxymethyl)-3-methyldihydrofuran-2(3H)-one, 20 g, 122.1 mmol) in pyridine (200 mL) was added benzoyl chloride (86.8 g, 617 mmol). The solution was stirred for 16 h at R.T. The reaction was quenched by the addition of MeOH (50 mL). The mixture was concentrated under reduced pressure, diluted with EA (1000 mL) and washed with NaHCO₃ (aq., 2×500 mL). The solution was dried over anhydrous Na₂SO₄, filtered and concentrated under reduced pressure. The residue was applied onto a silica gel column with EA/PE (1:2) to provide compound E ((3R,4R,5R)-5-((benzoyloxy)methyl)-3-methyl-2-oxotetra hydrofuran-3,4-diyldibenzoate, 50 g, 82%) as a white solid. ESI-MS: m/z 475 [M+H]⁺.

Compound F: To a solution of compound E (60 g, 120 mmol) in THF (400 mL) was added LiAl(t-BuO)₃H (1M in THF, 189.7 mL). The solution was stirred for 4 h at R.T., quenched by the addition of 1 N HCl (2000 mL), and extracted EA (2×2000 mL). The organic layers were combined, washed with NaHCO₃ (aq., 2000 mL). The solution was dried over anhydrous Na₂SO₄, filtered and concentrated under reduced pressure to provide compound F ((3R,4R,5R)-5-((benzoyloxy)methyl)-2-hydroxy-3-methyltetrahydrofuran-3,4-diyldibenzoate, crude, 60 g) as a colorless oil. ESI-MS: m/z: 477 [M+H]⁺.

Intermediate 2: To a solution of compound F (65 g, 129.6 mmol) in pyridine (600 mL) was added benzoyl chloride (57.3 g, 407.6 mmol) at R.T. The solution was stirred for 4 h at 60° C. The reaction was quenched by the addition MeOH (50 mL). The solution was concentrated under reduced pressure and then diluted with EA (1000 mL), washed with NaHCO₃ (aq., 2×500 mL). The solution was dried over anhydrous Na₂SO₄, filtered and concentrated under reduced pressure. The residue was applied onto a silica gel column with EA/PE (1:4) to provide Intermediate 2 ((3R,4R,5R)-5-((benzoyloxy)methyl)-3-methyltetrahydrofuran-2,3,4-triyltribenzoate, 70 g, 88%) as a yellow solid. ESI-MS: m/z 603 [M+N]⁺.

### Intermediate 3

### ((2R,3R,4R)-3-(benzoyloxy)-4-fluoro-5-hydroxy-4-methyltetrahydrofuran-2-yl)methyl benzoate

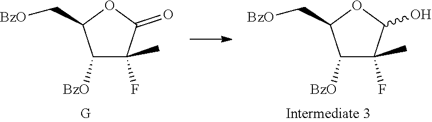

Intermediate 3 was prepared according to Reddy et al., J. Org. Chem. (2011) 76(10), 3782-3790, which is hereby incorporated by reference for the limited purpose of the preparation of Intermediate 3. To a solution of compound G ((2R,3R,4R)-3-(benzoyloxy)-4-fluoro-4-methyl-5-oxotetrahydrofuran-2-yl)methylbenzoate, 10 g, 26.9 mmol. See Wang et al., J. Org. Chem. (2009) 74(17):6819-6824) in THF (46 mL) was added lithium tri-tert-butoxyaluminohydride (1 M in THF, 40 mL) at −20° C. The resulting solution was stirred for 1 h at −20° C. The reaction was quenched with EA (100 mL) followed by saturated aq. NH₄Cl (10 mL) below 0° C. The result solution was diluted with 150 mL of EA, washed with 200 mL of 3N HCl and 200 mL of saturated aq. NaHCO₃, dried over anhydrous Na₂SO₄, filtered and concentrated under reduced pressure. The residue was applied onto a silica gel column with EA/PE (2:3), which provided Intermediate 3 ((2R,3R,4R)-3-(benzoyloxy)-4-fluoro-5-hydroxy-4-methyltetrahydrofuran-2-yl)methylbenzoate, 9.28 g (92%, α/β=1/3)) as a colorless oil.

### Intermediate 4

### ((2R,3R,4R,5R)-3-(benzoyloxy)-5-bromo-4-fluoro-4-methyltetrahydrofuran-2-yl)methyl benzoate

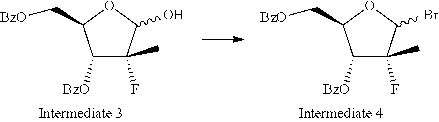

### Intermediate 3 ((2R,3R,4R)-3-(benzoyloxy)-4-fluoro-5-hydroxy-4-methyltetrahydrofuran-2-yl)methylbenzoate) (α/β=1/3) stored at 50° C. for 48 h, α/β=1/3 changed α/β=1/20. To a solution of Intermediate 3 (5 g, 13.4 mmol, α/β=1/20) in DCM (50 mL) was added Ph₃P (4.9 g, 18.7 mmol) at −20° C. The resulting solution was stirred for 15 mins at −20° C. and tetrabromomethane (6.63 g, 20 mmol) was added at −20° C. The resulting solution was stirred for 5 h at −20° C., then quenched by the addition of silica gel (5 g) and filtered. The solution was concentrated under reduced pressure. The residue was applied onto a silica gel column with EA/PE (1:6). This resulted in 2.41 g (43%) of Intermediate 4 (((2R,3R,4R,5R)-3-(benzoyloxy)-5-bromo-4-fluoro-4-methyltetrahydrofuran-2-yl)methylbenzoate) as a colorless oil. ESI-MS: m/z 437, 439 [M+H]⁺.

### Intermediate 5

### 3,5-bis(methylthio)-1,2,4-triazin-6-amine

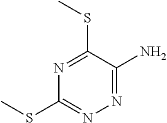

To a solution of 1,2,4-triazine-3,5(2H,4H)-dione (25.0 g, 221 mmol) in H₂O (350 mL) was added Br₂ (77.5 g, 485 mmol) drop-wise. The mixture was stirred at 25° C. for 24 h. The mixture was filtered to give a white solid. The solid was dried under reduced pressure. 6-bromo-1,2,4-triazine-3,5(2H,4H)-dione (40 g, 47.1% yield) was obtained as a white solid. ¹H NMR (400 MHz, DMSO-d₆) δ=12.55 (s, 1H), 12.29 (s, 1H).

6-bromo-1,2,4-triazine-3,5(2H,4H)-dione (10.0 g, 52.1 mmol) was treated with Cu (331 mg, 5.2 mmol, 37 μL) and NH₃ (50 mL) in sealed tube and the reaction was stirred at 80° C. for 48 h. The mixture was cooled up to −40° C. and NH₃ (liquid) was volatilized. The crude product was dissolved with hot H₂O (400 mL) and the resulting solution was adjusted to pH 4 with HCl. The resulting suspension was filtered, dissolved in dilute aq. NH₄OH and filtered again. The filtrate was acidified with HCl until a precipitate formed and the suspension was filtered to give a white solid. 6-amino-1,2,4-triazine-3,5(2H,4H)-dione (15.40 g, 120.2 mmol, 57.7% yield) was obtained as a white solid. ¹H NMR (400 MHz, DMSO-d₆) δ=11.72 (s, 1H), 10.87 (s, 1H), 5.94 (d, J=3.7 Hz, 2H).

To a solution of 6-amino-1,2,4-triazine-3,5(2H,4H)-dione (7.70 g, 60.1 mmol) in pyridine (500 mL) was added P₂S₅ (29.40 g, 132 mmol, 14.1 mL). The mixture was stirred at 130° C. for 7 h. Solvent was removed under reduced pressure and the residue was dissolved in H₂O (500 mL). The suspension was stirred at 100° C. and then allowed to stand for 18 h. The solid was collected by filtration, dissolved in H₂O (300 mL), and adjusted to pH 10 with NH₄OH. The solution was treated with norit, filtered, and the filtrate was acidified with HCl. After concentrating under reduced pressure, 6-amino-1,2,4-triazine-3,5(2H,4H)-dithione (10.0 g, 51.9% yield) was obtained as a brown solid. ¹H NMR (400 MHz, DMSO-d₆) δ=14.25 (s, 1H), 13.02 (s, 1H), 6.63 (s, 2H).

To a solution of 6-amino-1,2,4-triazine-3,5(2H,4H)-dithione (5.20 g, 32.5 mmol) in DCM (400 mL) was added DIEA (25.17 g, 194.8 mmol, 34.0 mL) and MeI (13.4 g, 94.4 mmol, 5.9 mL). The mixture was stirred at R.T. for 12 h. After concentrating under reduced pressure, the residue was purified on silica gel column with PE/EA (10:1-1:2). Intermediate 5 (3,5-bis(methylthio)-1,2,4-triazin-6-amine, 5.0 g, 26.6 mmol, 81.8% yield) was obtained as a yellow solid. ¹H NMR (400 MHz, CDCl₃) δ=4.65 (s, 2H), 2.60-2.61 (m, 6H).

### Example 1

### Compounds 1, 2 and 3

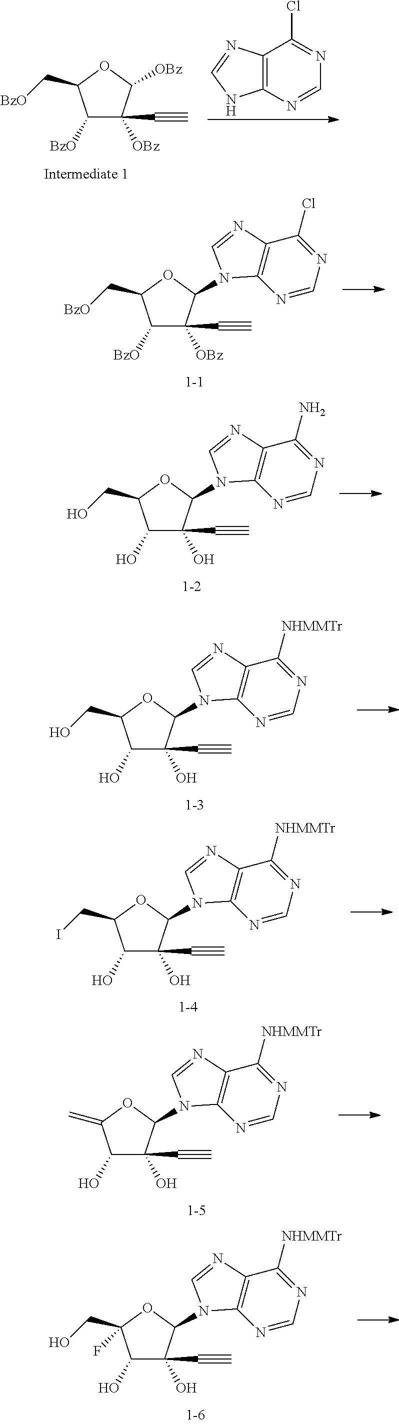

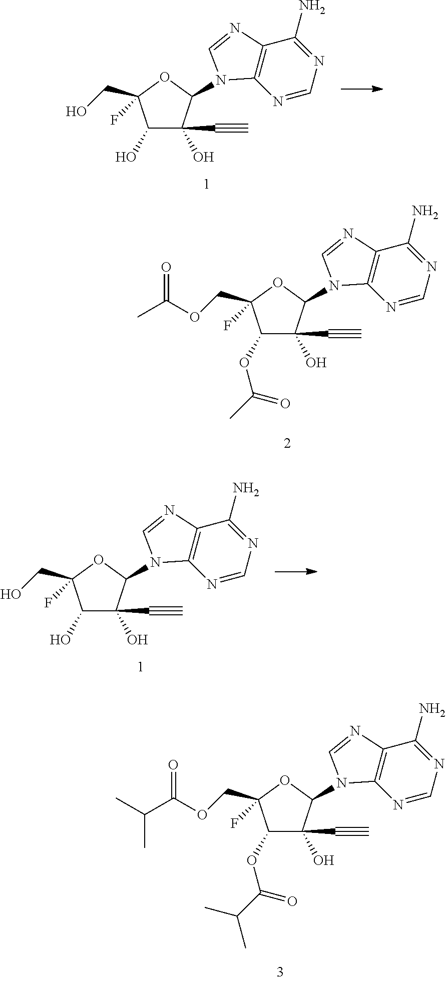 [SMILES: C#C[C@]1(O)[C@H](n2cnc3c(N)ncnc32)O[C@](F)(CO)[C@H]1O ; C#C[C@]1(O)[C@H](n2cnc3c(N)ncnc32)O[C@](F)(COC(C)=O)[C@H]1OC(C)=O ; C#C[C@]1(O)[C@H](n2cnc3c(N)ncnc32)O[C@](F)(CO)[C@H]1O ; C#C[C@]1(O)[C@H](n2cnc3c(N)ncnc32)O[C@](F)(COC(=O)C(C)C)[C@H]1OC(=O)C(C)C]

To a solution of 2R,3R,4R,5R)-5-((benzoyloxy)methyl)-3-ethynyltetrahydrofuran-2,3,4-triyltribenzoate (Intermediate 1, 4.0 g, 6.8 mmol) in ACN (40 mL) was added 6-chloro-9H-purine (2.09 g, 13.5 mmol) at R.T. DBU (5.88 g, 38.6 mmol) was then added at 0° C. The solution was stirred for 15 mins at 0° C. and then trimethylsilyl trifluoromethanesulfonate (12.05 g, 54.2 mmol) was added dropwise with stirring at 0° C. The solution was stirred for 15 mins at 0° C., then 16 h at 70° C. The solution was diluted with EA (500 mL) and washed with sat. NaHCO₃ solution (200 mL). The solution was dried over anhydrous Na₂SO₄, filtered and concentrated under reduced pressure. The residue was applied onto a silica gel column with EA:PE (1:5-1:3). Compound 1-1 was obtained ((2R,3R,4R,5R)-5-((benzoyloxy)methyl)-2-(6-chloro-9-yl)-3-ethynyltetrahydrofuran-3,4-diyldibenzoate, 1.5 g, 36%) as a yellow solid. MS m/z (ESI): 623 [M+H]⁺.

To a solution of compound 1-1 (1.5 g, 2.4 mmol) in 1,4-dioxane (15 mL) was added ammonia (30%, 30 mL). The solution was stirred for 12 h at 110° C. in sealed tube. The solution was cooled to R.T. and concentrated under reduced pressure. The residue was applied onto a silica gel column with EA:MeOH (30:1-10:1). Compound 1-2 was obtained ((2R,3R,4R,5R)-2-(6-amino-9H-purin-9-yl)-3-ethynyl-5-(hydroxymethyl)tetrahydrofuran-3,4-diol, 520 mg, 74%) as a yellow solid. MS m/z (ESI): 292 [M+H]⁺.

To a solution of compound 1-2 (5 g, 17.2 mmol) in pyridine (50 mL) was added trimethylchlorosilane (18.65 g, 171.7 mmol). The solution was stirred for 8 h at R.T. 4-methoxytriphenylmethyl chloride (26.45 g, 85.9 mmol) and 4-dimethylaminopyridine (415 mg, 3.4 mmol) were added. The solution was allowed to react for 24 h at 40° C. The solution was diluted with EA (500 mL), washed with water (500 mL) and dried over anhydrous Na₂SO₄. The solid was filtered off and the resulting solution was concentrated under reduced pressure. THE (50 mL) and tetrabutylammonium fluoride (1M in THF, 34.4 mL) were added and the reaction was allowed to proceed for 2 h at R.T. The solution was diluted with EA (500 mL) and washed with water (500 mL), dried over anhydrous Na₂SO₄, filtered and concentrated under reduced pressure. The residue was purified on silica gel with DCM:MeOH (40:1-20:1). Compound 1-3 was obtained ((2R,3R,4R,5R)-3-ethynyl-5-(hydroxymethyl)-2-(6-(((4-methoxyphenyl)diphenylmethyl)amino)-9H-purin-9-yl)tetrahydrofuran-3,4-diol, 5 g, 41%) as a white solid. MS m/z (ESI): 564.

To a solution of compound 1-3 (5 g, 8.9 mmol) and PPh₃ (2.79 g, 10.5 mmol) and imidazole (713.5 mg, 10.5 mmol) in THF (50 mL) was added a solution of iodine (2.47 g, 9.7 mmol) at 0° C. The solution was stirred for 2 h at R.T., diluted with EA (500 mL), and washed with sodium thiosulfate (aq) (500 mL). The solution was dried over anhydrous Na₂SO₄, filtered and concentrated under reduced pressure. The residue was purified on silica gel with DCM:MeOH (40:1). Compound 1-4 was obtained ((2R,3R,4R,5S)-3-ethynyl-5-(iodomethyl)-2-(6-(((4-methoxyphenyl)diphenylmethyl)amino)-9H-purin-9-yl)tetrahydrofuran-3,4-diol, 3.8 g, 51%) as a white solid. MS m/z (ESI): 674 [M+H]⁺.

A solution of compound 1-4 (3 g, 4.5 mmol) in 5% NaOMe in MeOH (30 mL) was stirred for 4 h at 60° C. The pH value of the solution was adjusted to 7 with acetic acid. The solution was concentrated under reduced pressure. The residue was applied onto a silica gel column with DCM:MeOH (40:1). Compound 1-5 was obtained ((2R,3R,4S)-3-ethynyl-2-(6-(((4-methoxyphenyl)diphenylmethyl)amino)-9H-purin-9-yl)-5-methylenetetrahydrofuran-3,4-diol, 1.5 g, 56%) as a white solid. MS m/z (ESI): 546 [M+H]⁺.

To a solution of compound 1-5 (500 mg, 0.9 mmol) in DCM (4 mL) was added a solution of 3-chloroperoxybenzoic acid (70%, 450 mg, 1.8 mmol) in DCM (2 mL) at 0° C. TEA●3HF (0.73 g, 4.5 mmol) was added at 0° C. The solution was stirred for 2 h at R.T. and then concentrated under reduced pressure. The residue was applied onto a silica gel column with DCM:MeOH (40:1). Compound 1-6 was obtained ((2S,3S,4R,5R)-4-ethynyl-2-fluoro-2-(hydroxymethyl)-5-(6-(((4-methoxyphenyl)diphenylmethyl)amino)-9H-purin-9-yl)tetrahydrofuran-3,4-diol, 87.5 mg, 15%) as a white solid. MS m/z (ESI): 582 [M+H]⁺.

To a solution of compound 1-6 (300 mg, 0.52 mmol) in dioxane (3 mL) was added 5% TFA (6 mL). The solution was stirred for 2 h at R.T. The pH value of the solution was adjusted to 8 with ammonia (30%). The solution was concentrated under reduced pressure. The crude product (300 mg) was purified by Prep-HPLC with the following conditions: Atlantis Prep T3 OBD Column, 19*250 mm 10 u; mobile phase, water/ACN (3-15% ACN in 12 min); Detector, uv 254 nm. Compound 1 was obtained ((2S,3S,4R,5R)-5-(6-amino-9H-purin-9-yl)-4-ethynyl-2-fluoro-2-(hydroxymethyl)tetrahydrofuran-3,4-diol, 70.1 mg, 42%) as a white solid. MS m/z (ESI): 310 [M+H]⁺.

To a solution of compound 1 (40 mg, 0.13 mmol) in pyridine (2.4 mL) was added acetic anhydride (52.8 mg, 0.52 mmol). The solution was stirred for 20 h at 25° C. The reaction was quenched by the addition MeOH (1 mL). After concentrating under reduced pressure, the residue was purified on silica gel with DCM:MeOH (10:1). Compound 2 was obtained (((2S,3S,4R,5R)-3-acetoxy-5-(6-amino-9H-purin-9-yl)-4-ethynyl-2-fluoro-4-hydroxytetrahydrofuran-2-yl)methyl acetate, 31.2 mg, 61%) as a white solid. MS m/z (ESI): 394 [M+H]⁺.

To a solution of compound 1 (50 mg, 0.16 mmol) in pyridine (3 mL) was added isobutyric anhydride (153.5 mg, 0.97 mmol). The solution was stirred for 48 h at R.T. The reaction was quenched by the addition of MeOH (1 mL). After concentrated under reduced pressure, the residue was applied onto a silica gel column with DCM:MeOH (10:1). Compound 3 was obtained ((2S,3S,4R,5R)-5-(6-amino-9H-purin-9-yl)-4-ethynyl-2-fluoro-4-hydroxy-2-((isobutyryloxy)methyl)tetrahydrofuran-3-ylisobutyrate, 37.5 mg, 52%) as a white solid. MS m/z (ESI): 450 [M+H]⁺.

### Example 2

### Compound 4: (2R,3R,4R,5R)-2-(6-amino-2-fluoro-9H-purin-9-yl)-3-ethynyl-5-(hydroxymethyl)tetrahydrofuran-3,4-diol

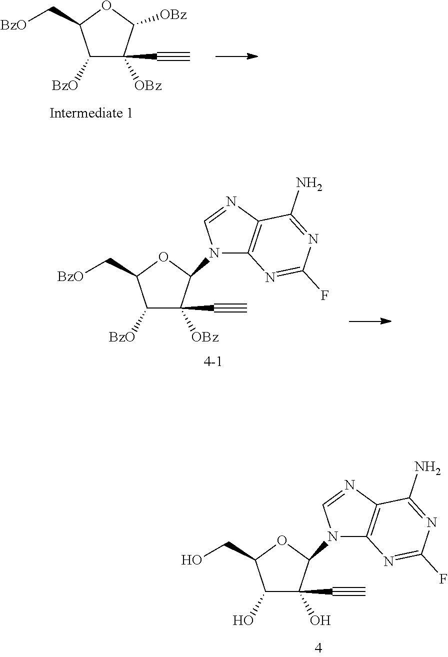

2-fluoroadenosine (1.6 g, 10.4 mmol) was co-evaporated with anhydrous toluene (3×5 mL) and was then suspended in 1,2-DCE (60 mL). 1,8-diazabicyclo[5.4.0]undec-7-ene (DBU, 2.01 mL, 13.9 mmol, 2.0 eq.) and trimethylsilyl trifluoromethanesulfonate (TMSOTf, 7.6 mL, 41.8 mmol) were added. The mixture was heated to 65° C. for 30 mins. Intermediate 1 (4.1 g, 7 mmol, 1.0 eq.) in 1,2-DCE (40 mL), added at 65° C. After stirring at 65° C. for 10 mins, the mixture was refluxed (100° C.) for 18 h. The mixture was cooled R.T. The solution was diluted with EA (250 mL), washed with sat. NaHCO₃ solution (1×50 mL), filtered, dried over anhydrous Na₂SO₄, and concentrated under reduced pressure. The crude residue was purified by silica gel chromatography (0-80% EA in hexane, v/v) to afford compound 4-1 ((2R,3R,4R,5R)-2-(6-amino-2-fluoro-9H-purin-9-yl)-5-((benzoyloxy)methyl)-3-ethynyl)tetrahydrofuran-3,4-diyldibenzoate, 3.1 g, 72%) as a white solid. MS m/z (ESI): 622.15 [M+H]⁺.

Compound 4-2 (150 mg, 0.24 mmol) was suspended in NH₃/MeOH (6N, 10 mL) and the mixture was heated to 55° C. for 16 h. The mixture was then evaporated to dryness. The crude residue was purified by silica gel chromatography (3-25% MeOH in DCM, v/v) to afford compound 4 ((2R,3R,4R,5R)-2-(6-amino-2-fluoro-9H-purin-9-yl)-3-ethynyl-5-(hydroxymethyl)tetrahydrofuran-3,4-diol, 44 mg, 59%) as a white solid. MS m/z (ESI): 310 [M+H]⁺.

### Example 3

### Compound 5: (2R,3R,4R,5R)-2-(2,6-diamino-9H-purin-9-yl)-3-ethynyl-5-(hydroxymethyl)tetrahydrofuran-3,4-diol

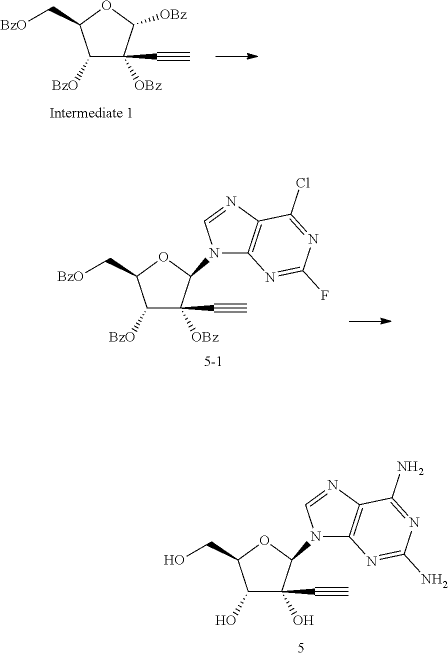

Intermediate 1 (500 mg, 0.85 mmol) was co-evaporated with anhydrous toluene (3×5 mL) and dissolved in anhydrous ACN (5 mL). 2-fluoro-6-chloro-9H-purine (292 mg, 1.7 mmol) was added at R.T. 1,8-diazabicyclo[5.4.0]undec-7-ene (721 μL, 4.8 mmol) was added at 0° C. The solution was stirred for 15 min at 0° C. Trimethylsilyl trifluoromethanesulfonate (1.2 mL, 6.8 mmol) was added dropwise with stirring at 0° C. The solution was stirred for 15 mins at 0° C., warmed to 70° C. and stirred for 18 h. The solution was cooled to R.T., the solution was diluted with EA (50 mL), washed with sat. NaHCO₃ (1×15 mL) and dried over anhydrous Na₂SO₄. The crude residue was purified on silica gel (0-50% EA in hexane, v/v) to afford compound 5-1 ((2R,3R,4R,5R)-5-((benzoyloxy)methyl)-2-(6-chloro-2-fluoro-9H-purin-9-yl)-3-ethynyltetrahydrofuran-3,4-diyldibenzoate, 349 mg, 65%) as a white solid. MS m/z (ESI): 641.15 [M+H]⁺.

Compound 5-1 (45 mg, 0.07 mmol) was suspended in NH₃/MeOH (6N, 6 mL) and the mixture was heated to 110° C. for 28 h. The mixture was evaporated to dryness and purified by Prep-HPLC (Buffer A: 50 mM TEAA in H₂O, Buffer B: 50 mM TEAA in ACN, with liner gradient increase of 0-30% in 20 min) to afford compound 5 ((2R,3R,4R,5R)-2-(2,6-diamino-9H-purin-9-yl)-3-ethynyl-5-(hydroxymethyl)tetrahydrofuran-3,4-diol, 10.3 mgs, 46%) as a white solid. MS m/z (ESI): 307 [M+H]⁺.

### Example 4

### Compound 6: (2S,3S,4R,5R)-5-(4-amino-7H-pyrrolo[2,3-d]pyrimidin-7-yl)-4-ethynyl-2-fluoro-2-(hydroxymethyl)tetrahydrofuran-3,4-diol

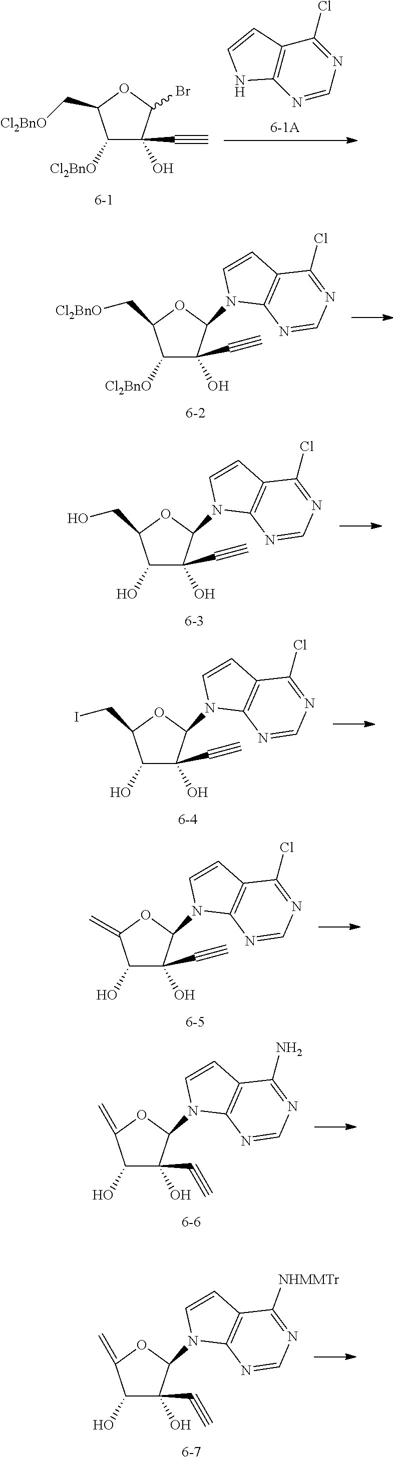

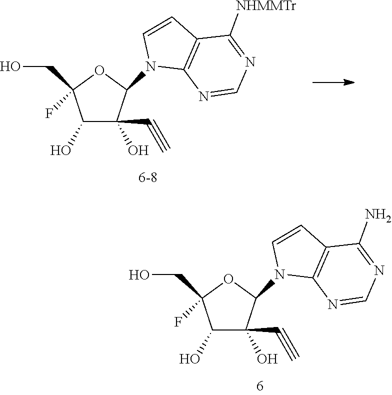

To a solution of compound 6-1A (4-chloro-7H-pyrrolo[2,3-d]pyrimidine, 2.21 g, 14.4 mmol) in ACN (300 mL) was added NaH (2.88 g, 72.1 mmol, 60% purity) at 25° C. The mixture stirred for 30 mins and compound 6-1 (((3R,4R,5R)-2-bromo-4-((2,4-dichlorobenzyl)oxy)-5-(((2,4-dichlorobenzyl)oxy)methyl)-3-ethynyltetrahydrofuran-3-ol, 8.0 g, 14.4 mmol, prepared as described in WO 2010/015643, which is hereby incorporated by reference for the particular purpose of its description for preparing compound 6-1) was added. The mixture was stirred at 25° C. for 12 h. The reaction was quenched by the addition of 10% citric acid solution (20 mL) and the solution was concentrated under reduced pressure. The residue was dissolved with DCM (100 mL). The solution was washed with H₂O (2×100 mL), dried over anhydrous NaSO₄, filtered and concentrated under reduced pressure. The residue was purified by column chromatography (PE:EA=40:1-5:1) to give compound 6-2 (5.5 g, 60.8%) as a yellow solid.

To a solution of compound 6-2 ((2R,3R,4R,5R)-2-(4-chloro-7H-pyrrolo[2,3-d]pyrimidin-7-yl)-4-((2,4-dichlorobenzyl)oxy)-5-(((2,4-dichlorobenzyl)oxy)methyl)-3-ethynyltetrahydrofuran-3-ol, 4.20 g, 6.7 mmol) in DCM (40 mL) was added BCl₃ (1 M, 8.71 mL) at −78° C. The mixture was stirred at 0° C. for 1 h. The reaction was quenched with isopropanol (15 mL) and stirred for 30 mins. The mixture was concentrated to dryness. The residue was purified by column chromatography (DCM:MeOH=50:1-5:1) to give compound 6-3 ((2R,3R,4R,5R)-2-(4-chloro-7H-pyrrolo[2,3-d]pyrimidin-7-yl)-3-ethynyl-5-(hydroxymethyl)tetrahydrofuran-3,4-diol, 1.5 g, 72.4%) as a white solid. ESI-MS: m/z=309.8 [M+1]⁻.

To a solution of compound 6-3 (185 mg, 597.35 μmol) in THF (2 mL) was added 12 (151.61 mg, 597.35 μmol), PPh₃ (313 mg, 1.2 mmol) and imidazole (81.3 mg, 1.2 mmol). The mixture was stirred at 25° C. for 12 h. The reaction was quenched by the addition of sat. Na₂S₂O₃ solution (2 mL) and extracted with EA (3×10 mL). The organic layer was concentrated under reduced pressure. The residue was purified by column chromatography (PE:EA=20:1-5:1) to give compound 6-4 ((2R,3R,4R,5S)-2-(4-chloro-7H-pyrrolo[2,3-d]pyrimidin-7-yl)-3-ethynyl-5-(iodomethyl)tetrahydrofuran-3,4-diol, 170 mg, 67.82% as a white solid. ESI-MS: m/z=419.8 [M+1]⁺.

To a solution of compound 6-4 (2.0 g, 4.8 mmol) in THF (20 mL) was added DBU (10.89 g, 71.6 mmol) at 0° C. The mixture was stirred at 25° C. for 5 h. The mixture was adjusted to pH 7 by the addition of a HOAc solution and extracted with EA (40 mL). The organic layer was concentrated under reduced pressure. The residue was purified by column chromatography (PE:EA=20:1-5:1) to give compound 6-5 ((2R,3R,4S)-2-(4-chloro-7H-pyrrolo[2,3-d]pyrimidin-7-yl)-3-ethynyl-5-methylenetetrahydrofuran-3,4-diol, 900.0 mg, 60.1%) as a white solid.

Compound 6-5 (810 mg, 2.8 mmol) was subjected to NH₃₍ₗ₎ at 90° C. for 11 h. The ammonia was removed and the residue purified on silica gel (3-15% MeOH/DCM, v/v) to afford compound 6-6 ((2R,3R,4S)-2-(4-amino-7H-pyrrolo[2,3-d]pyrimidin-7-yl)-3-ethynyl-5-methylenetetrahydrofuran-3,4-diol, 625 mg, 82%) as a white solid. MS m/z [M+H]⁺ (ESI): 272.95.

Compound 6-6 (590 mg, 2.2 mmol) was co-evaporated with anhydrous pyridine (2×20 mL) and dissolved in anhydrous pyridine (25 mL). Monomethoxytrityl chloride (1.46 g, 4.8 mmol) was added at R.T. After stirring at 45° C. for 20 h, the mixture was diluted EA (50 mL) and washed with sat. aq. NaHCO₃ (20 mL) and sat. NaCl (20 mL). The crude was purified by column chromatography (0-80% EA in hexane, v/v) to afford compound 6-7 ((2R,3R,4S)-3-ethynyl-2-(4-(((4-methoxyphenyl)diphenylmethyl)amino)-7H-pyrrolo[2,3-d]pyrimidin-7-yl)-5-methylenetetrahydrofuran-3,4-diol, 675 mg, 58%) as a white solid. MS m/z [M+H]⁺ (ESI): 545.10.

Compound 6-7 (470 mg, 0.86 mmol) was co-evaporated with anhydrous toluene (2×20 mL) and dissolved in anhydrous DCM (6 mL). The mixture was cooled to 0° C. A solution of 3-chloroperoxybenzoic acid (70%, 297 mg, 1.7 mmol) in DCM (2 mL) was added, followed by TEA●3HF (0.71 mL, 4.3 mmol) at 0° C. The solution was stirred for 2 h at R.T. and then concentrated under reduced pressure. The crude was purified by column chromatography (0-10% MeOH in DCM, v/v) to afford compound 6-8 ((2S,3S,4R,5R)-4-ethynyl-2-fluoro-2-(hydroxymethyl)-5-(4-(((4-methoxyphenyl)diphenylmethyl)amino)-7H-pyrrolo[2,3-d]pyrimidin-7-yl)tetrahydrofuran-3,4-diol, 75 mg, 15%) as a white solid. MS m/z (ESI): 581.10 [M+H]⁺.

Compound 6-8 (102 mg, 0.18 mmol) was subjected to HCl in ACN (0.525 mmol, 0.4M, 1.3 mL). After stirring at R.T. for 8 h, the solution was evaporated to dryness and purified on silica gel (3-25% MeOH in DCM, v/v) to afford compound 6 ((2S,3S,4R,5R)-5-(4-amino-7H-pyrrolo[2,3-d]pyrimidin-7-yl)-4-ethynyl-2-fluoro-2-(hydroxymethyl)tetrahydrofuran-3,4-diol, 25.4 mg, 48%) as a white solid. MS m/z (ESI): 308.95 [M+H]⁺.

The structures of compounds 1-6 are summarized in Table 2 below.

TABLE 2

| No. | Structure |
|---|---|
| 1 | 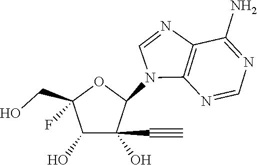 [SMILES: C#C[C@]1(O)[C@H](n2cnc3c(N)ncnc32)O[C@](F)(CO)[C@H]1O] |
| 2 | 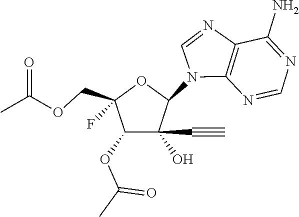 [SMILES: C#C[C@]1(O)[C@H](n2cnc3c(N)ncnc32)O[C@](F)(COC(C)=O)[C@H]1OC(C)=O] |
| 3 | 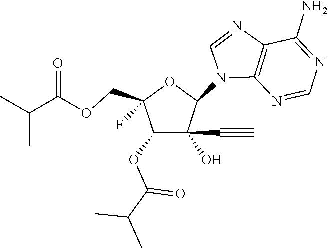 [SMILES: C#C[C@]1(O)[C@H](n2cnc3c(N)ncnc32)O[C@](F)(COC(=O)C(C)C)[C@H]1OC(=O)C(C)C] |
| 4 | 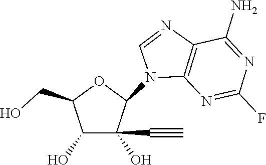 [SMILES: C#C[C@@]1(O)[C@H](O)[C@@H](CO)O[C@H]1n1cnc2c(N)nc(F)nc21] |
| 5 | 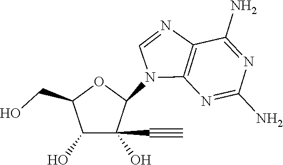 [SMILES: C#C[C@@]1(O)[C@H](O)[C@@H](CO)O[C@H]1n1cnc2c(N)nc(N)nc21] |
| 6 | 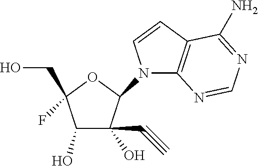 [SMILES: C#C[C@]1(O)[C@H](n2ccc3c(N)ncnc32)O[C@](F)(CO)[C@H]1O] |

### Example 5

### Compound 7: (2S,3S,4R,5R)-5-(4-amino-5-fluoro-7H-pyrrolo[2,3-d]pyrimidin-7-yl)-4-ethynyl-2-fluoro-2-(hydroxymethyl)tetrahydrofuran-3,4-diol

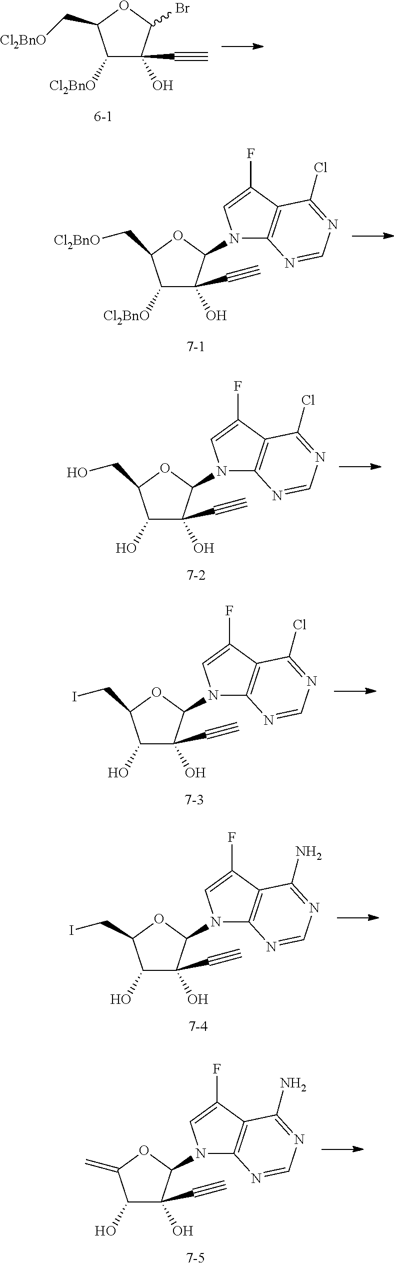

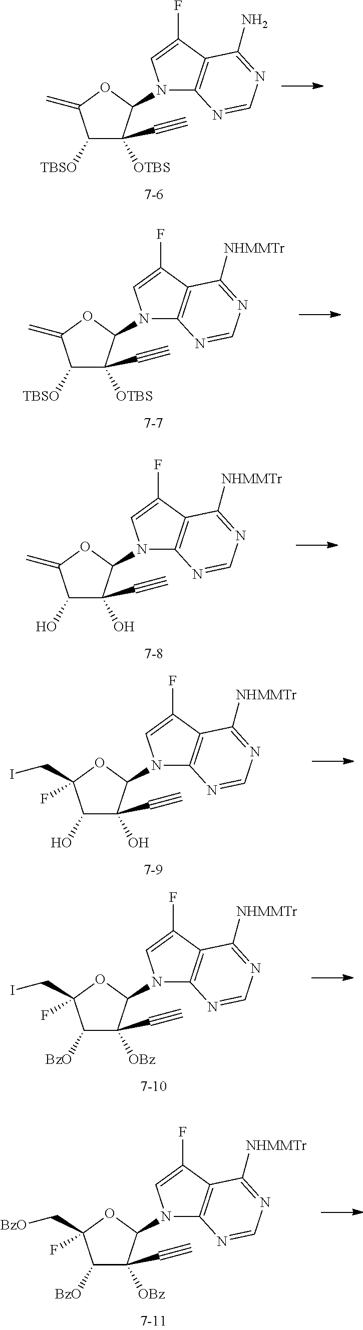

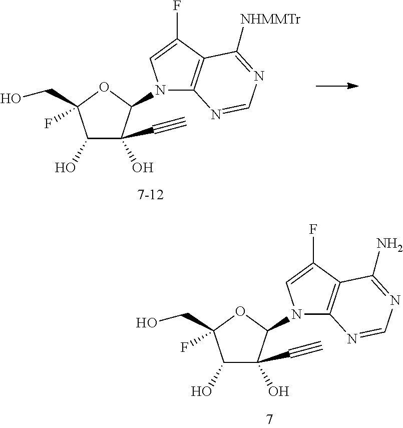

To a suspension of 4-chloro-5-fluoro-7H-pyrrolo[2,3-d]pyrimidine (2.29 g, 13.33 mmol, 1 eq.) in ACN (135.00 mL) was added NaH (1.60 g, 40 mmol, 60% purity, 3.00 eq.) in one portion at R.T. under N₂. The mixture was stirred at R.T. for 1 h, then a solution of compound 6-1 ((3R,4R,5R)-2-bromo-4-((2,4-dichlorobenzyl)oxy)-5-(((2,4-dichlorobenzyl)oxy)methyl)-3-ethynyltetrahydrofuran-3-ol, 7.40 g, 13.33 mmol, 1 eq.) in ACN (130 mL) was added. The reaction was stirred at 25° C. for 4 h, neutralized with saturated aqueous citric acid (to pH 7) and diluted with EA (160 mL) and water (40 mL). The aqueous phase was extracted with EA (80 mL*2) and the combined organic phase was washed with brine (50 mL*2), dried with anhydrous NaSO₄, filtered and concentrated in vacuum. The residue was purified by column chromatography (SiO₂, PE/EA=20/1 to 3/1) to give compound 7-1 ((2R,3R,4R,5R)-2-(4-chloro-5-fluoro-7H-pyrrolo[2,3-d]pyrimidin-7-yl)-4-((2,4-dichlorobenzyl)oxy)-5-4(2,4-dichlorobenzyl)oxy)methyl)-3-ethynyltetrahydrofuran-3-ol, 5.60 g, crude) as a brown oil, which was further purified using preparative HPLC to provide (2 g, 35.8%) of compound 7-1 as a white solid. LCMS: ESI-MS: m/z=643.8 [M+H]⁺.

To a solution of 7-1 (2.00 g, 3.10 mmol, 1 eq.) in DCM (25.00 mL) was added BCl₃ (1 M, 24.80 mL, 8 eq.) drop-wise at −78° C. under N₂. The mixture was stirred at 0° C. for 2 h and then quenched with i-PrOH (8 mL) at 0° C. and neutralized with NH₃ H₂O to pH 7. The mixture was concentrated under reduced pressure and the residue was purified by column chromatography (SiO₂, DCM/MeOH=20/1 to 5/1) to give 7-2 ((2R,3R,4R,5R)-2-(4-chloro-5-fluoro-7H-pyrrolo[2,3-d]pyrimidin-7-yl)-3-ethynyl-5-(hydroxymethyl)tetrahydrofuran-3,4-diol, 700 mg, 2.14 mmol, 68.9%) as a white solid. ¹⁹F NMR (MeOD, 376 MHz) δ=−170.78. LCMS: ESI-MS: m/z=327.9 [M+H]⁺.

To a solution of Compound 7-2 (1.17 g, 3.57 mmol, 1 eq.) in THF (20.00 mL) was added PPh₃ (1.87 g, 7.14 mmol, 2 eq.) and imidazole (486.14 mg, 7.14 mmol, 2.00 eq.) in one portion, followed by drop-wise a solution of I₂ (1.36 g, 5.36 mmol, 1.08 mL, 1.50 eq.) in THF (20.00 mL). The reaction mixture was stirred at R.T. for 2 h. The reaction mixture was quenched by saturated NaS₂O₃ (5 mL) and diluted with EA (30 mL) and water (20 mL). The aqueous phase was extracted with ethyl acetate (25 mL*2). The combined organic phase was washed with brine (5 mL), dried over anhydrous Na₂SO₄, filtered and concentrated at low pressure. The residue was purified by column chromatography (SiO₂, PE:EA=8/1 to 2.5/1) to give compound 7-3 ((2R,3R,4R,5S)-2-(4-chloro-5-fluoro-7H-pyrrolo[2,3-d]pyrimidin-7-yl)-3-ethynyl-5-(iodomethyl)tetrahydrofuran-3,4-diol, 1.40 g, 3.20 mmol, 89.6%, 100% purity) as the white solid. LCMS: ESI-MS: m/z=438.0 [M+H]⁺.

Compound 7-3 (Batch 1, 2.20 g, 5.03 mmol, 1 eq.) was dissolved in liquid NH₃ (60 mL) and then the mixture was stirred at 40° C. for 1.5 h in sealed tube. The mixture was concentrated under reduced pressure and the residue was purified by silica gel chromatography (Eluent of 0˜5% MeOH/DCM ether). 731 mg of mixture of compound 7-4 ((2R,3R,4R,5S)-2-(4-amino-5-fluoro-7H-pyrrolo[2,3-d]pyrimidin-7-yl)-3-ethynyl-5-(iodomethyl)tetrahydrofuran-3,4-diol, 73% purity) and compound 7-5 ((2R,3R,4S)-2-(4-amino-5-fluoro-7H-pyrrolo [2,3-d]pyrimidin-7-yl)-3-ethynyl-5-methylenetetrahydrofuran-3,4-diol, 21% purity) was obtained as a white solid.

Compound 7-3 (Batch 2, 2.20 g, 5.03 mmol, 1.00 eq.) was dissolved in liquid NH₃ (60.00 mL) and then the mixture was stirred at 40° C. for 1.5 h in sealed tube. The mixture was concentrated under reduced pressure. The residue was purified by silica gel chromatography (Eluent of 0˜5% MeOH/DCM ether). 711 mg of mixture of compound 7-4 ((2R,3R,4R,5S)-2-(4-amino-5-fluoro-7H-pyrrolo[2,3-d]pyrimidin-7-yl)-3-ethynyl-5-(iodomethyl)tetrahydrofuran-3,4-diol, 73% purity) and compound 7-5 ((2R,3R,4S)-2-(4-amino-7H-pyrrolo[2,3-d]pyrimidin-7-yl)-3-ethynyl-5-methylenetetrahydrofuran-3,4-diol, 21% purity) was obtained as a white solid. Batches 1 and 2 of compound 7-4 (1.44 g, 73% purity) were used to next step directly without further purification. LCMS: ESI-MS: m/z=419.1 [M+H]⁺.

To a solution of crude compound 7-4 (1.44 g, 2.51 mmol, 1 eq.) in THF (17 mL) was added DBU (1.91 g, 12.57 mmol, 1.89 mL, 5 eq.). The mixture was stirred at R.T. for 16 h. The reaction was neutralized with AcOH to pH 7, and concentrated under reduced pressure. The residue was purified by silica gel chromatography (Eluent of 90% (EA/ACN=10:1)/petroleum ether gradient) to give compound 7-5 ((2R,3R,4S)-2-(4-amino-5-fluoro-7H-pyrrolo[2,3-d]pyrimidin-7-yl)-3-ethynyl-5-methylenetetrahydrofuran-3,4-diol, 830 mg, 92% purity, 28% over two steps) as a white solid. LCMS: ESI-MS: m/z=291.0 [M+H]⁺.

To a solution of compound 7-5 (1.24 g, 4.27 mmol, 1 eq.) in DMF (5 mL) was added imidazole (1.74 g, 25.62 mmol, 6 eq.) and TBSCl (2.57 g, 17.08 mmol, 2.09 mL, 4 eq.). The mixture was stirred at 60° C. for 16 h. The reaction mixture was partitioned between H₂O (100 mL) and EA (150 mL). The organic phase was separated, washed with brine (100 mL), dried over MgSO₄, filtered and concentrated under reduced pressure. The residue was purified by silica gel chromatography (Eluent of 35% EA/petroleum ether) to give compound 7-6 (7-((2R,3R,4R)-3,4-bis((tert-butyldimethylsilyl)oxy)-3-ethynyl-5-methylenetetrahydrofuran-2-yl)-5-fluoro-7H-pyrrolo[2,3-d]pyrimidin-4-amine, 1.42 g, 2.74 mmol, 64.1%, 100% purity) as a white solid. LCMS: ESI-MS: m/z=519.1 [M+H]⁺.

To a solution of compound 7-6 (1.42 g, 2.74 mmol, 1.00 eq.) in pyridine (9 mL) was added DMAP (167.20 mg, 1.37 mmol, 0.5 eq.) and MMTrCl (2.11 g, 6.84 mmol, 2.5 eq.). The mixture was stirred at 60° C. for 16 h. The reaction mixture was partitioned between H₂O (30 mL) and EA (50 mL). The organic phase was separated, washed with brine (30 mL), dried over MgSO₄, filtered and concentrated under reduced pressure. The residue was purified by silica gel chromatography (Eluent of 15% EA/petroleum ether) to give compound 7-7 (7-((2R,3R,4R)-3,4-bis((tert-butyldimethylsilyl)oxy)-3-ethynyl-5-methylenetetrahydrofuran-2-fluoro-N-((4-methoxyphenyl)diphenylmethyl)-7H-pyrrolo[2,3-d]pyrimidin-4-amine, 2.06 g, 2.50 mmol, 91.2%, 96% purity) as a white solid. LCMS: ESI-MS: m/z=791.3 [M+H]⁺.

To a solution of compound 7-7 (2.80 g, 3.54 mmol, 1 eq.) in THF (10 mL) was added TBAF (1 M, 10.62 mL, 3 eq.). The mixture was stirred at R.T. for 15 min. The mixture was concentrated under reduced pressure. The residue was purified by silica gel chromatography (Eluent of 50-85% EA/Petroleum ether) to give compound 7-8 ((2R,3R,4S)-3-ethynyl-2-(5-fluoro-4-(((4-methoxyphenyl)diphenylmethyl)amino)-7H-pyrrolo[2,3-d]pyrimidin-7-yl)-5-methylenetetrahydrofuran-3,4-diol, 1.85 g, 3.19 mmol, 90.1%, 97% purity) as a white solid. LCMS: ESI-MS: m/z=563.3 [M+H]⁺.

A solution of compound 7-8 (1.24 g, 2.20 mmol, 1 eq.) in ACN (15 mL) was treated with N,N-diethylethanamine trihydrofluoride (532 mg, 3.30 mmol, 537.4 μL, 1.5 eq.) and NIS (1.24 g, 5.50 mmol, 2.5 eq.). The mixture was cooled to 0° C. and stirred at 0° C. for 1.5 h. The mixture was concentrated at under reduced pressure. The residue was purified by silica gel chromatography (Eluent of 1˜40% EA/petroleum ether) to give crude product. The crude product was purified by Prep-HPLC (FA system) to give compound 7-9 ((2R,3S,4R,5R)-4-ethynyl-2-fluoro-5-(5-fluoro-4-(((4-methoxyphenyl)diphenylmethyl)amino)-7H-pyrrolo[2,3-d]pyrimidin-7-yl)-2-(iodomethyl)tetrahydrofuran-3,4-diol, 468 mg, 594.5 μmol, 13.5%) as a yellow solid. ¹⁹F NMR(376 MHz, CD₃OD) δ−111.37, −171.05. LCMS: ESI-MS: m/z=709.1 [M+H]₊.

A solution of compound 7-9 (468 mg, 661 μmol, 1 eq.) in pyridine (4.4 mL) was treated with DMAP (40.4 mg, 330.3 μmol, 0.5 eq.) and benzoylbenzoate (598 mg, 2.64 mmol, 498 μL, 4 eq.). The mixture was stirred at 60° C. for 3 h, then quenched by addition of saturated NaHCO₃ (30 mL) at 20° C. and extracted with EA (45 mL). The organic layer was washed with brine (35 mL), dried over MgSO₄, filtered and concentrated under reduced pressure. The residue was purified by silica gel chromatography (Eluent of 0˜15% EA/petroleum ether) to give compound 7-10 ((2R,3S,4R,5R)-4-ethynyl-2-fluoro-5-5-fluoro-4-(((4-methoxyphenyl)diphenylmethyl)amino)-7H-pyrrolo[2,3-d]pyrimidin-7-yl)-2-(iodomethyl)tetrahydrofuran-3,4-diyldibenzoate, 490 mg, 534.53 μmol, 80.9%, 100% purity) as a white solid. ¹⁹F NMR (CD₃OD, 376 MHz) δ=−104.84, −168.27. LCMS: ESI-MS: m/z=917.0 [M+H]⁺, 939.4 [M+Na]⁺.

To a solution of compound 7-10 (490 mg, 534.5 μmol, 1 eq.) in DMF (13 mL) was added 15-crown-5 (1.30 g, 5.88 mmol, 1.17 mL, 11 eq.) and benzoyloxysodium (770.3 mg, 5.35 mmol, 1.60 mL, 10 eq.). The mixture was stirred at 105° C. for 36 h. The mixture was filtered and then diluted with EA (100 mL). The combined organic layers were washed with H₂O (100 mL), saturated NaHCO₃ (100 mL), brine (100 mL), dried over Na₂SO₄, filtered and concentrated under reduced pressure. The residue was purified by silica gel chromatography (Eluent of 15% EA/petroleum ether gradient) to give compound 7-11 ((2S,3S,4R,5R)-2-((benzoyloxy)methyl)-4-ethynyl-2-fluoro-5-(5-fluoro-4-(((4-methoxyphenyl)diphenylmethyl)amino)-7H-pyrrolo[2,3-d]pyrimidin-7-yl)tetrahydrofuran-3,4-diyldibenzoate, 280 mg, 307.4 μmol, 57.5%, 100% purity) as a white solid. LCMS: ESI-MS: m/z=911.1 [M+H]⁺.

Compound 7-11 (280.00 mg, 307.38 μmol, 1 eq.) was dissolved in THF (2 mL) and butan-1-amine (3.70 g, 50.59 mmol, 5 mL, 164.6 eq.). The mixture was stirred at R.T. for 16 h. The mixture was concentrated under reduced pressure. The residue was purified by silica gel chromatography (Eluent of 50% EA/petroleum ether) to give compound 7-12 ((2S,3S,4R,5R)-4-ethynyl-2-fluoro-5-(5-fluoro-4-(((4-methoxyphenyl)diphenylmethyl)amino)-7H-pyrrolo[2,3-d]pyrimidin-7-yl)-2-(hydroxymethyl)tetrahydrofuran-3,4-diol, 152 mg, 253.9 μmol, 82.6%, 100% purity) as a white solid.

Compound 7-12 (152 mg, 253.9 μmol, 1 eq.) was treated with 80% AcOH (1.50 mL), stirred at 20° C. for 6 h, then concentrated under reduced pressure. The residue was purified by silica gel chromatography (Eluent of 90% EA/Petroleum ether) to give crude product. The crude product was purified by Prep-HPLC (FA system) to give compound 7 ((2S,3 S,4R,5R)-5-(4-amino-5-fluoro-7H-pyrrolo[2,3-d]pyrimidin-7-yl)-4-ethynyl-2-fluoro-2-(hydroxymethyl)tetrahydrofuran-3,4-diol, 44 mg, 133.5 μmol, 52.5%, 99% purity) as a white solid. ¹H NMR (400 MHz, CD₃OD) δ 8.10 (s, 1H), 7.26 (d, J=2.0 Hz, 1H), 6.63 (s, 1H), 4.63 (br, d, J=19.1 Hz, 1H), 3.82-3.72 (m, 2H), 2.63 (s, 1H). ¹⁹F NMR (376 MHz, CD₃OD) δ−123.69, −169.71. LCMS: ESI-MS: m/z=326.9 [M+H]⁺.

### Example 6

### Compound 8: (2S,3S,4R,5R)-5-(7-amino-5-fluoro-3H-imidazo[4,5-b]pyridin-3-yl)-4-ethynyl-2-fluoro-2-(hydroxymethyl)tetrahydrofuran-3,4-diol

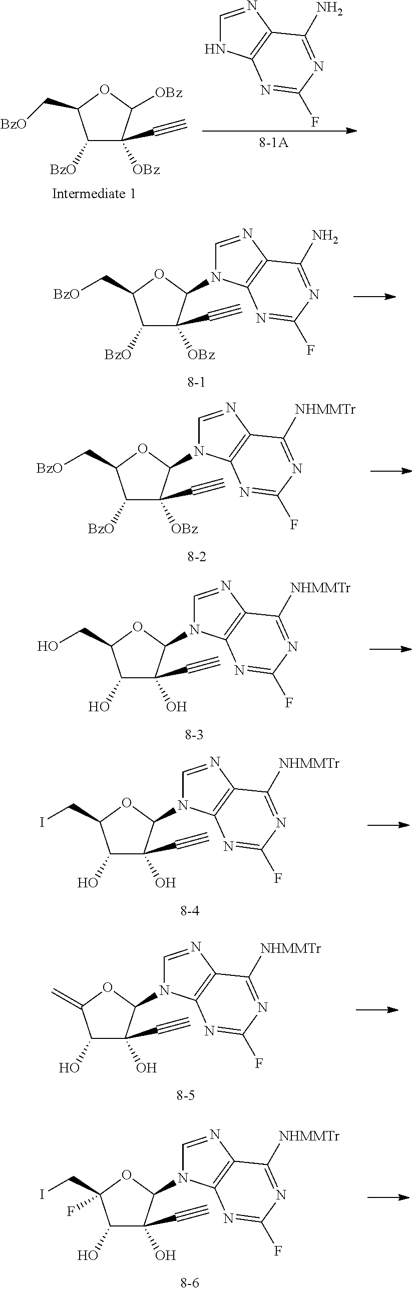

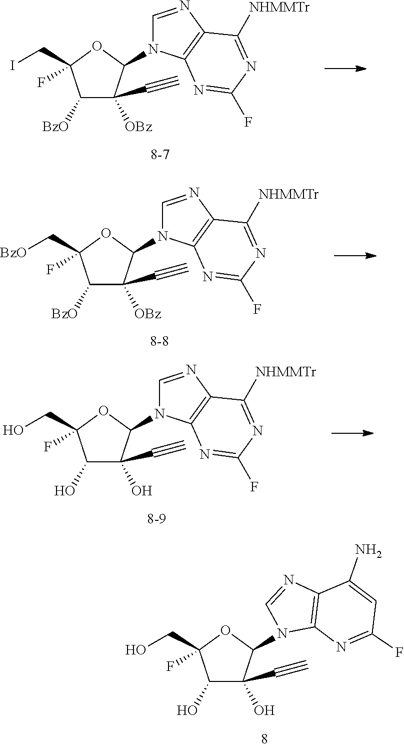

To a solution of Intermediate 1 (1.59 g, 10.41 mmol) in DCE (29.00 mL) was added DBU (2.11 g, 13.88 mmol) and TMSOTf (9.26 g, 41.65 mmol, 7.53 mL). The mixture was heated at 65° C. for 0.5 h. Compound 8-1A (2-fluoro-9H-purin-6-amine, 4.1 g, 6.94 mmol) in DCE (19.00 mL) was added into the mixture. The resulting mixture was stirred at 100° C. for 18 h, then diluted with EA (100 mL), washed with sat. NaHCO₃ solution (100 mL) and dried over anhydrous Na₂SO₄, filtered, and concentrated under reduced pressure. The residue was purified by column chromatography (petroleum ether/EA, 10/1 to 1/1) to give Compound 8-1 ((2R,3R,4R,5R)-2-(6-amino-2-fluoro-9H-purin-9-yl)-5-((benzoyloxy)methyl)-3-ethynyltetrahydrofuran-3,4-diyldibenzoate, 6.94 g, 72.4%, 90% purity) as a yellow solid. LCMS: ESI-MS: m/z 622.1 [M+H]⁺.

To a solution of compound 8-1 (5.7 g, 9.17 mmol) in pyridine (30.5 mL) was added 4-methoxytriphenylmethyl chloride (MMTrCl, 7.08 g, 22.93 mmol) and DMAP (560.17 mg, 4.59 mmol). The mixture was stirred at 60° C. for 40 h and then diluted with EA (250 mL). The mixture was washed with sat. NaHCO₃ solution (150 mL) and dried over anhydrous MgSO₄, filtered, and concentrated under reduced pressure. The residue was purified by column chromatography (petroleum ether/EA, 20/1 to 1/1) to give compound 8-2 ((2R,3R,4R,5R)-5-((benzoyloxy)methyl)-3-ethynyl-2-(2-fluoro-6-(((4-methoxyphenyl)diphenylmethyl)amino)-9H-purin-9-yl)tetrahydrofuran-3,4-diyldibenzoate, 3.7 g, 41.1%, 91% purity) as a yellow solid. ESI-MS: m/z 894.2 [M+H]⁺, 916.0 [M+Na]⁺.

Compound 8-2 (1.8 g, 2.01 mmol) in NH₃ (7M in MeOH, 122.45 mL) was stirred at 50° C. for 12 h. The mixture was concentrated under reduced pressure and the residue purified by column chromatography (DCM/MeOH, 100/1 to 10/1) to give compound 8-3 ((2R,3R,4R,5R)-3-ethynyl-2-(2-fluoro-6-(((4-methoxyphenyl)diphenylmethyl)amino)-9H-purin-9-yl)-5-(hydroxymethyl)tetrahydrofuran-3,4-diol, 2.1 g, 79.8%, 89% purity) as a white solid. ESI-MS: m/z=582.1 [M+H]⁺.

To a solution of compound 8-3 (2.1 g, 3.61 mmol) in THF (2.7 mL) and pyridine (4 mL) was added PPh₃ (1.70 g, 6.50 mmol) and imidazole (491.64 mg, 7.22 mmol). I₂ (1.37 g, 5.42 mmol, 1.09 mL) in THF (16 mL) was added to the mixture, which was then stirred at 30° C. for 16 h. The mixture was extracted with EA (15 mL) and washed with saturated sodium thiosulfate solution (15 mL). The organic phase was dried over anhydrous Na₂SO₄, and concentrated under reduced pressure. The residue was purified by column chromatography (DCM/EA, 20/3 to 5/1) to give compound 8-4 ((2R,3R,4R,5S)-3-ethynyl-2-(2-fluoro-6-(((4-methoxyphenyl)diphenylmethyl)amino)-9H-purin-9-yl)-5-(iodomethyl)tetrahydrofuran-3,4-diol, 1.78 g, 67.7%, 95% purity) as a faint yellow solid. LCMS: ESI-MS: m/z 692.1 [M+H]⁺.

To a solution of compound 8-4 (1.78 g, 2.57 mmol) in THF (10 mL) was added DBL (1.96 g, 12.87 mmol, 1.94 mL), and stirred at R.T. for 12 h. The mixture was neutralized with AcOH (2 mL), and concentrated under reduced pressure. The residue was purified by column chromatography (petroleum ether/EA, 3/1 to 1/3) to give compound 8-5 ((2R,3R,4S)-3-ethynyl-2-(2-fluoro-6-(((4-methoxyphenyl)diphenylmethyl)amino)-9H-purin-9-yl)-5-methylenetetrahydrofuran-3,4-diol, 1.33 g, 91.7%, 100% purity) as a colorless oil. ESI-MS: m/z=586.1 [M+Na]⁺.

To a solution of compound 8-5 (1.3 g, 2.31 mmol) in ACN (13 mL) was added N,N-diethylethanamine trihydrofluoride (371.9 mg, 375.6 μL) at 0° C., and NIS (778.44 mg, 3.46 mmol, 1.50 eq.), and stirred at 0° C. for 2 h. The mixture was extracted with EA (30 mL), and washed with saturated sodium thiosulfate solution (25 mL) and saturated K₂CO₃ solution (25 mL). The organic phase was dried over anhydrous Na₂SO₄ and concentrated under reduced pressure. The residue was purified by column chromatography (petroleum ether/EA, 20/1 to 1/2) to give compound 8-6 ((2R,3S,4R,5R)-4-ethynyl-2-fluoro-5-(2-fluoro-6-(((4-methoxyphenyl)diphenylmethyl)amino)-9H-purin-9-yl)-2-(iodomethyl)tetrahydrofuran-3,4-diol, 1.43 g, 80.4%, 92% purity) as a white solid. ESI-MS: m/z=710.1 [M+H]⁺.

To a solution of compound 8-6 (1.43 g, 2.0 mmol) in pyridine (14 mL) was added DMAP (123.12 mg, 1.01 mmol) and benzoylbenzoate (1.37 g, 6.05 mmol, 1.14 mL), and stirred at 65° C. for 3 h. The mixture was extracted with EA (50 mL), and washed with the saturated solution of NH₄Cl (50 mL) and saturated solution of NaHCO₃ (80 mL). The organic phase was dried over anhydrous Na₂SO₄, and concentrated under reduced pressure. The residue was purified by column chromatography (petroleum ether/EA, 100/1 to 3/1) to give compound 8-7 ((2R,3S,4R,5R)-4-ethynyl-2-fluoro-5-(2-fluoro-6-(((4-methoxyphenyl)diphenylmethyl)amino)-9H-purin-9-yl)-2-(iodomethyl)tetrahydrofuran-3,4-diyldibenzoate, 1.1 g, 59.4%, 99.8% purity) as a white solid. LCMS: ESI-MS: m/z=918.2 [M+H]⁺, 940.2 [M+Na]⁺.

To a solution of compound 8-7 (142 mg, 154.7 μmol) in DMSO (3 mL) was added sodium benzoate (222.98 mg, 1.55 mmol) and 15-crown-5 (374.90 mg, 1.70 mmol), and the mixture was stirred at 105° C. for 12 h. The mixture was diluted with EA (20 mL), filtered on the celite, and the filtrate washed with H₂O (20 mL) and brine (20 mL) and dried over anhydrous Na₂SO₄. The resulting solution was concentrated under reduced pressure. The residue was purified by column chromatography (petroleum ether/EA, 20/1 to 1/1) to give compound 8-8 ((2S,3S,4R,5R)-2-((benzoyloxy)methyl)-4-ethynyl-2-fluoro-5-(2-fluoro-6-(((4-methoxyphenyl)diphenylmethyl)amino)-9H-purin-9-yl)tetrahydrofuran-3,4-diyldibenzoate, 63 mg, 40.6%, 91% purity) as light yellow solid. LCMS: ESI-MS: m/z 912.2 [M+H]⁺, 935.2 [M+Na]⁺.

Compound 8-8 (110 mg, 120.6 μmol) was treated with NH₃/MeOH (5 mL, 7.0 M). The mixture was stirred at R.T. for 12 h. The reaction mixture was concentrated under reduced pressure. The residue was purified by prep-HPLC (column: Phenomenex Kinetex XB-C18 150 mm*30 mm, 5 μm; mobile phase: [water (10 mM NH₄HCO₃)— ACN]; B %: 40%-70%, 12 min) to give compound 8-9 ((2S,3S,4R,5R)-4-ethynyl-2-fluoro-5-(2-fluoro-6-(((4-methoxyphenyl)diphenylmethyl)amino)-9H-purin-9-yl)-2-(hydroxymethyl)tetrahydrofuran-3,4-diol, 41 mg, 55%, 97% purity) as light yellow oil. LCMS: ESI-MS: m/z 600.1 [M+H]⁺.

Compound 8-9 (40 mg, 66.71 μmol) was dissolved in a mixture of AcOH (0.8 mL) and H₂O (0.2 mL) and stirred at 20° C. for 1 h. The mixture was diluted with MeOH (5 mL) and concentrated under reduced pressure. The residue was purified by column chromatography (DCM/MeOH, 60/1 to 20/1) to give compound 8 ((2S,3S,4R,5R)-5-(7-amino-5-fluoro-3H-imidazo[4,5-b]pyridin-3-yl)-4-ethynyl-2-fluoro-2-(hydroxymethyl)tetrahydrofuran-3,4-diol, 16.3 mg, 72.5%, 97.08% purity) as white solid. ¹H NMR (400 MHz, CD₃CN) δ=8.09 (s, 1H), 6.41 (br, s, 2H), 6.32 (s, 1H), 4.84-4.79 (m, 1H), 4.75 (s, 1H), 4.21 (br, d, J=9.5 Hz, 1H), 3.91-3.88 (m, 1H), 3.81-3.78 (m, 2H), 2.52 (s, 1H). MS: ESI-MS: m/z32 328.08 [M+H]⁺.

### Example 7

### Compound 9: 4-amino-7-((2R,3R,4S,5S)-3-ethynyl-5-fluoro-3,4-dihydroxy-5-(hydroxymethyl)tetrahydrofuran-2-yl)-7H-pyrrolo[2,3-d]pyrimidine-5-carbonitrile

### Compound 10: 4-amino-7-((2R,3R,4S,5S)-3-ethynyl-5-fluoro-3,4-dihydroxy-5-(hydroxymethyl)tetrahydrofuran-2-yl)-7H-pyrrolo[2,3-d]pyrimidine-5-carboxamide

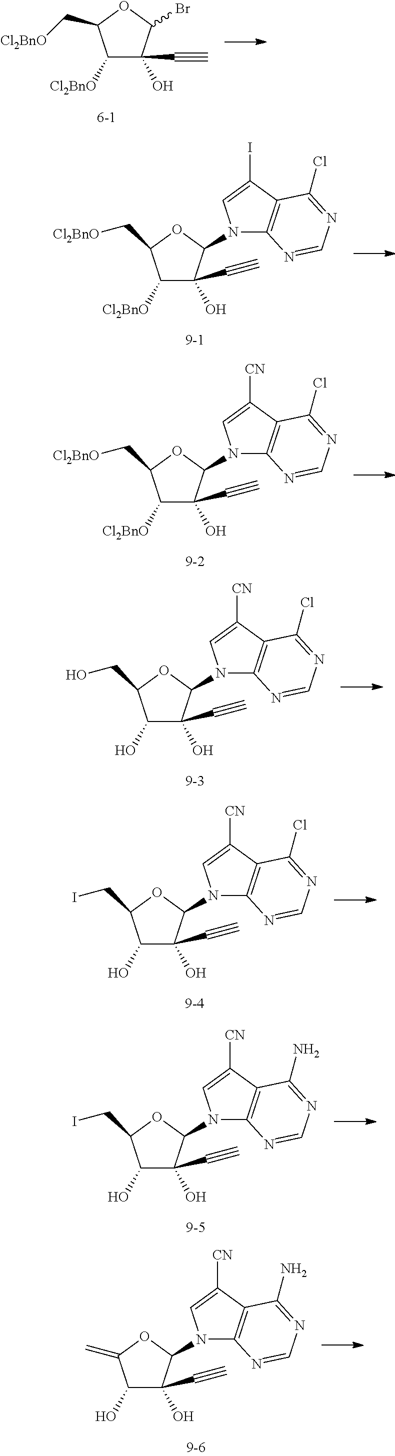

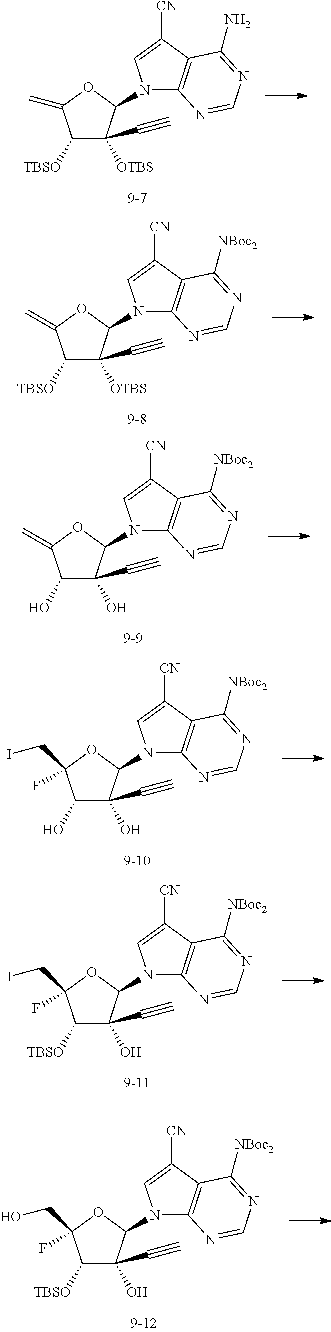

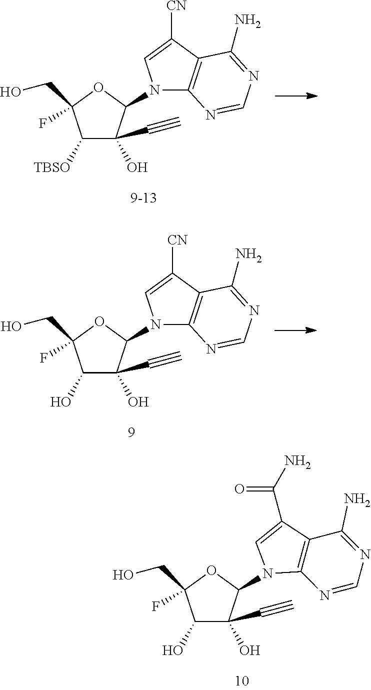 [SMILES: C#C[C@]1(O)[C@H](n2cc(C#N)c3c(N)ncnc32)O[C@](F)(CO)[C@H]1O[Si](C)(C)C(C)(C)C ; C#C[C@]1(O)[C@H](n2cc(C#N)c3c(N)ncnc32)O[C@](F)(CO)[C@H]1O ; C#C[C@]1(O)[C@H](n2cc(C(N)=O)c3c(N)ncnc32)O[C@](F)(CO)[C@H]1O]

To a suspension of 4-chloro-5-iodo-7H-pyrrolo[2,3-d]pyrimidine (1.13 g, 4.05 mmol, 1 eq.) in ACN (40.00 mL) was added NaH (729.00 mg, 12.15 mmol, 40% purity, 3 eq.) in one portion at R.T. under N₂. The mixture was stirred at R.T. for 1 h, then a solution of compound 6-1 ((3R,4R,5R)-2-bromo-4-((2,4-dichlorobenzyl)oxy)-5-(((2,4-dichlorobenzyl)oxy)methyl)-3-ethynyltetrahydrofuran-3-ol, 2.25 g, 4.05 mmol, 1.00 eq.) in ACN (40.00 mL) was added to the mixture in one portion. The reaction was stirred at R.T. for 12 h, then diluted with EA (160 mL) and water (40 mL) and neutralized with saturated NaHCO₃. The aqueous phase was extracted with EA (100 mL*2) and the combined organic phases were washed with brine (50 mL*2), dried over anhydrous Na₂SO₄, filtered and concentrated under reduced pressure. The residue was purified by column chromatography (SiO₂, petroleum ether/EA, 8/1 to 1/1) to give compound 9-1 ((2S,3S,4R,5R)-2-((R)-4-chloro-5-iodo-7H-cyclopenta[d]pyrimidin-7-yl)-4-((2,4-dichlorobenzyl)oxy)-5-(((2,4-dichlorobenzyl)oxy)methyl)-3-ethynyltetrahydrofuran-3-ol, 2.00 g, 2.65 mmol, 32.76%) as a white solid.

To a solution of compound 9-1 (2.35 g, 3.12 mmol, 1 eq.) in DMF (24 mL) was added Zn(CN)₂ (915 mg, 7.80 mmol, 494.8 μL, 2.5 eq.) and Pd(PPh₃)₄ (1.08 g, 936 μmol, 0.30 eq.) in one portion at R.T. under N₂. The mixture was stirred at 90° C. for 1.5 h. The mixture was cooled to R.T. and 6 batches product was combined together to work up. The combined mixture was diluted with EA (450 mL), filtrated on celite and the filter cake was washed with EA (2×50 mL). The filtrate was diluted with brine (200 mL) and water (200 mL). The aqueous phase was extracted with EA (2×150 mL). The combined organic phase was washed with brine (2×50 mL), dried over anhydrous Na₂SO₄, filtered and concentrated under reduced pressure. The residue was purified by column chromatography (SiO₂, petroleum ether:EA, 20:1 to 5:1) to give compound 9-2 ((R)-4-chloro-7-((2S,3S,4R,5R)-4-((2,4-dichlorobenzyl)oxy)-5-(((2,4-dichlorobenzyl)oxy)methyl)-3-ethynyl-3-hydroxytetrahydrofuran-2-yl)-7H-cyclopenta[d]pyrimidine-5-carbonitrile, 7.00 g, 10.72 mmol, 57.29%, 100% purity) as light oil. LCMS: m/z=652.9 [M+H]⁺.

To a solution of compound 9-2 (2.65 g, 4.06 mmol) in DCM (35 mL) was added BCl₃ (1 M, 32.48 mL) dropwise at −78° C. under N₂. The mixture was stirred at 0° C. for 2 h. Three batches were combined for work up. The reaction mixture was quenched with iPrOH (40 mL) at 0° C. and the mixture was neutralized with NH₃/H₂O to pH 7. The mixture was concentrated under reduced pressure and the residue was purified by column chromatography (SiO₂, DCM/MeOH, 20/1 to 5/1) to give compound 9-3 (4-chloro-7-((2R,3R,4R,5R)-3-ethynyl-3,4-dihydroxy-5-(hydroxymethyl)tetrahydrofuran-2-yl)-7H-pyrrolo[2,3-d]pyrimidine-5-carbonitrile, 3.65 g, 89.53%) as a white solid.

To a solution of compound 9-3 (1.4 g, 4.18 mmol) in THF (40 mL) was added imidazole (569.52 mg, 8.36 mmol) and PPh₃ (2.19 g, 8.36 mmol) in one portion, followed by dropwise a solution of I₂ (1.59 g, 6.27 mmol) in THF (20 mL). The mixture was stirred at R.T. for 2 h, then quenched with saturated Na₂S₂O₃ (8 mL) and the mixture was diluted with EA (80 mL) and water (20 mL). The aqueous phase was extracted with EA (45 mL*2) and the combined organic phase was washed with brine (35 mL), dried over anhydrous Na₂SO₄, filtered and concentrated under reduced pressure. The residue was purified by column chromatography (SiO₂, PE/EA=8/1 to 2.5/1) to give compound 9-4 (4-chloro-7-((2R,3R,4R,5S)-3-ethynyl-3,4-dihydroxy-5-(iodomethyl)tetrahydrofuran-2-yl)-7H-pyrrolo[2,3-d]pyrimidine-5-carbonitrile, 4.25 g, 76.23%) as brown solid.

Compound 9-4 (2.1 g, 4.72 mmol) was treated with liquid NH₃ (40 mL) and the reaction was stirred at R.T. for 1.5 h in a sealed tube. The mixture was concentrated under reduced pressure. The residue was purified by column chromatography (SiO₂, petroleum ether/EA, 1/1 to 1/9) to give compound 9-5 (4-amino-7-((2R,3R,4R,5S)-3-ethynyl-3,4-dihydroxy-5-(iodomethyl)tetrahydrofuran-2-yl)-7H-pyrrolo[2,3-d]pyrimidine-5-carbonitrile, 3.5 g, 87.2%) as a brown solid.

To a solution of compound 9-5 (1.75 g, 4.12 mmol) in THF (17.5 mL) was added DBU (3.14 g, 20.6 mmol, 3.11 mL) in portions at R.T. under N₂. The mixture was stirred at R.T. for 16 h. The mixture was neutralized with AcOH in THF (4 mL) to pH 7. The mixture was concentrated under reduced pressure. The residue was purified by column chromatography (SiO₂, petroleum ether/EA, 1/1 to 1/9) to give compound 9-6 (4-amino-7-((2R,3R,4S)-3-ethynyl-3,4-dihydroxy-5-methylenetetrahydrofuran-2-yl)-7H-pyrrolo[2,3-d]pyrimidine-5-carbonitrile, 2 g, 80.8%) as a white solid. LCMS: ESI-MS: m/z=297.9 [M+H]⁻.

To a solution of compound 9-6 (1 g, 3.36 mmol) in DMF (5 mL) was added imidazole (1.37 g, 20.16 mmol) and TBSC1 (2.03 g, 13.44 mmol) in one portion at R.T. under N₂. The mixture was stirred at 55° C. for 12 h. The mixture was cooled to R.T. and diluted with EA (80 mL) and water (20 mL). The aqueous phase was extracted with EA (30 mL*2). The combined organic phases were washed with brine (20 mL*2), dried over anhydrous Na₂SO₄, filtered and concentrated under reduced pressure. The residue was purified by silica gel chromatography (PE/EA=10/1 to 3/1) to give compound 9-7 (4-amino-7-((2R,3R,4R)-3,4-bi s((tert-butyldimethylsilyl)oxy)-3-ethynyl-5-methylenetetrahydrofuran-2-yl)-7H-pyrrolo[2,3-d]pyrimidine-5-carbonitrile, 2.38 g, 4.53 mmol, 67.4%) as a white solid. LCMS: ESI-MS: m/z=526.2 [M+H]⁺

To a solution of compound 9-7 (1.19 g, 2.26 mmol) in THF (30 mL) was added DMAP (55.3 mg, 452.65 μmol) and Boc₂O (1.48 g, 679 mmol) in one portion at R.T. under N₂. The mixture was stirred at R.T. for 12 h. The solvent was removed under reduced pressure and the residue was purified by column chromatography (SiO₂, petroleum ether/EA, 15/1 to 5/1) to give compound 9-8 (2.9 g, 78.6%) as brown oil. LCMS: ESI-MS: m/z=748.3 [M+Na]⁺.

To solution of compound 9-8 (1.45 g, 2.0 mmol) in THF (800 μL) was added TBAF (1 M, 7.99 mL) in one portion at R.T. under N₂. The mixture was stirred at R.T. for 15 min. The reaction mixture was removed under reduce pressure. The residue was purified by column chromatography (SiO₂, petroleum ether/EA, 5/1 to 1/1) to give compound 9-9 (1.56 g, 78.4%) as white solid. LCMS: ESI-MS: m/z=520.1 [M+Na]⁺.

To a solution of compound 9-9 (Batch 1, 200 mg, 402 μmol) in DCM (3 mL) was added Et₃N-3HF (64.8 mg, 402 μmol, 223 μL) and NIS (135.6 mg, 603 μmol) in one portion at −30° C. under N₂. The mixture was stirred at −30° C. for 2 h, then quenched with a mixture of saturated NaHCO₃ (5 mL) and saturated Na₂S₂O₃ (5 mL). The mixture was diluted with EA (30 mL). The aqueous phase was extracted with EA (15 mL*2) and the combined organic phases were washed with brine (30 mL), dried over anhydrous Na₂SO₄, filtered and concentrated under reduced pressure. The residue was purified by column chromatography (SiO₂, petroleum ether/EA, 10/1 to 1/1) to give compound 9-10 (190 mg, 295.3 μmol) as a brown solid. LCMS indicated that the product contained two isomers and the ratio was about 5:1.

To a solution of compound 9-9 (Batch 2, 680 mg, 1.37 mmol) in DCM (11 mL) was added Et₃N—3HF (220.9 mg, 1.37 mmol, 223 μL) and NIS (462.33 mg, 2.06 mmol) in one portion at −30° C. under N₂. The mixture was stirred at −30° C. for 2 h, then quenched with a mixture of saturated NaHCO₃ (10 mL) and saturated Na₂S₂O₃ (10 mL). The mixture was diluted with EA (80 mL). The aqueous phase was extracted with EA (35 mL*2) and the combined organic phases were washed with brine (30 mL), dried over anhydrous Na₂SO₄, filtered and concentrated under reduced pressure. The residue was purified by column chromatography (SiO₂, petroleum ether/EA, 10/1 to 1/1) to give compound 9-10 (1.45 g, 2.25 mmol) as a brown solid.

Batches 1 and 2 of compound 9-10 were combined and purified by Prep-HPLC (FA system) (column: Phenomenex Gemini C18 250*50 10 u; mobile phase: [water (0.225% FA)-ACN]; B %: 35%-65%, 11.2 min) to give compound 9-10 (395 mg, 613.93 μmol, 53.85%) as a white solid. LCMS: ESI-MS: m/z=487.9 [M+Na]⁻.

To a solution of compound 9-10 (800 mg, 1.24 mmol, 1 eq.) in DMF (2.5 mL) was added imidazole (338.60 mg, 4.97 mmol, 4 eq.) and TBSC1 (562.2 mg, 3.73 mmol, 457 μL, 3 eq.) in one portion at R.T. under N₂. The mixture was stirred at 50° C. for 2 h. The mixture was cooled to R.T. and diluted with EA (100 mL) and water (40 mL). The aqueous phase was extracted with EA (2×30 mL). The combined organic phases were washed with brine (20 mL), dried over anhydrous Na₂SO₄, filtered and concentrated under reduced pressure. The residue was purified by silica gel chromatography to give compound 9-11 (813 mg, 944.3 μmol, 76.15%, 88% purity) as a white solid. LCMS: ESI-MS: m/z=780.1 [M+Na]⁺.

A solution of tetrabutylammonium hydroxide (6.07 g, 12.87 mmol, 7.59 mL, 55% purity, 24 eq.) was neutralized with TFA (2.37 g, 20.75 mmol, 1.54 mL, 39.3 eq.) to pH=3˜4 at 0° C. and the mixture was added to the solution of compound 9-11 (406 mg, 535.9 μmol, 1 eq.) in DCM (6 mL). 3-chlorobenzenecarboperoxoic acid (759.2 mg, 2.64 mmol, 60% purity, 5 eq.) was added at 0° C. under vigorous stirring and the reaction was stirred at R.T. for 24 h. The reaction was quenched with saturated NaHCO₃ (15 mL) and saturated Na₂S₂O₃ (15 mL) at 0° C. The aqueous phase was extracted with EA (2×50 mL). The combined organic phases were washed with brine (20 mL), dried over anhydrous Na₂SO₄, filtered and concentrated under reduced pressure. The residue was purified by silica gel chromatography to give compound 9-12 (505 mg, 779.6 μmol, 72.7%) as a white solid. LCMS: ESI-MS: m/z=670.2 [M+Na]⁺.

To a solution of compound 9-12 (252 mg, 389 μmol, 1 eq.) in ACN (400 μL) was added a mixture of formic acid (1.83 g, 39.76 mmol, 1.50 mL) and H₂O (500 mg, 27.75 mmol, 500 μL) in one portion at R.T. under N₂. The reaction was stirred at R.T. for 8 h. The mixture was concentrated under reduced pressure. The residue was purified by silica gel chromatography to give compound 9-13 (4-amino-7-((2R,3R,4S,5S)-4-((tert-butyldimethylsilyl)oxy)-3-ethynyl-5-fluoro-3-hydroxy-5-(hydroxymethyl)tetrahydrofuran-2-yl)-7H-pyrrolo[2,3-d]pyrimidine-5-carbonitrile, 260 mg, 581 μmol, 74.7%) as a white solid. LCMS: ESI-MS: m/z=448.1 [M+H]⁺.

Compound 9-13 (130 mg, 290.5 μmol, 1 eq.) in THF (1 mL) was treated with TBAF (1 M, 435.7 μL, 1.5 eq.) in one portion at R.T. under N₂. The mixture was stirred at R.T. for 20 min. The mixture was concentrated under reduced pressure. The residue was purified by silica gel chromatography to give compound 9 (4-amino-7-((2R,3R,4S,5S)-3-ethynyl-5-fluoro-3,4-dihydroxy-5-(hydroxymethyl)tetrahydrofuran-2-yl)-7H-pyrrolo[2,3-d]pyrimidine-5-carbonitrile, 185 mg, 527.4 μmol, 90.8%, 95% purity) as a white solid. Compound 9 (35 mg, 105 μmol) was purified by prep-HPLC again (column: Phenomenex Gemini C18 250*50 10u; mobile phase: [water (0.225% FA)-ACN]; B %: 1%-26%, 11.2 min) to give compound 9 (22 mg, 64.7 μmol, 61.6%, 98% purity) as a white solid. ¹⁹F NMR (376 MHz, CD₃OD) δ=124.12. ¹H NMR (400 MHz, MeOD) δ=8.24 (s, 1H), 8.22 (s, 1H), 6.64 (s, 1H), 4.69 (s, 1H), 3.83-3.79 (m, 1H), 3.85-3.77 (m, 1H), 2.67 (s, 1H).

Compound 9 (Batch 1, 50 mg, 150 μmol, 1 eq.) was dissolved in a mixture of MeOH (230 μL), H₂O (448 mg, 3.96 mmol, 380 μL, 30% purity) and NH₃—H₂O (3.41 g, 27.26 mmol, 3.75 mL, 28% purity) in one portion at R.T. under N₂. The reaction was stirred at R.T. for 20 min. The solvent was removed under reduced pressure. The residue was purified by silica gel chromatography to give crude compound 10 (4-amino-7-((2R,3R,4S,5S)-3-ethynyl-5-fluoro-3,4-dihydroxy-5-(hydroxymethyl)tetrahydrofuran-2-yl)-7H-pyrrolo[2,3-d]pyrimidine-5-carboxamide, 40 mg, 108.2 μmol, 72.1%, 95% purity) as a light oil.

Compound 9 (Batch 2, 100 mg, 300 μmol, 1 eq.) was dissolved in a mixture of MeOH (460 μL), H₂O (896.80 mg, 7.91 mmol, 760 μL, 30% purity) and NH₃-H₂O (6.83 g, 54.52 mmol, 7.50 mL, 28% purity) in one portion at R.T. under N₂. The reaction was stirred at R.T. for 20 mins. The mixture was concentrated under reduced pressure. The residue was purified by silica gel chromatography to give crude compound 10 (4-amino-7-((2R,3R,4S,5S)-3-ethynyl-5-fluoro-3,4-dihydroxy-5-(hydroxymethyl)tetrahydrofuran-2-yl)-7H-pyrrolo[2,3-d]pyrimidine-5-carboxamide, 80 mg, 227.7 μmol, 75.9%) as light oil.

Batches 1 and 2 of compound 10 (115 mg, 1.14 mmol) were combined and purified by Prep-HPLC (FA system) to give compound 10 (60 mg, 170.8 μmol, 52.2%) as a white solid. ¹⁹F NMR (376 MHz, CD₃OD): δ=−124.73. ¹H NMR (400 MHz, CD₃OD) δ=8.14-8.12 (m, 1H), 8.05-8.01 (m, 1H), 6.64 (s, 1H), 4.72 (br, d, J=19.2 Hz, 1H), 3.86-3.81 (m, 1H), 3.87-3.80 (m, 1H), 2.69 (s, 1H). LCMS: ESI-MS: m/z=351.1 [M+H]⁺.

### Example 8

### Compound 11: (2S,3S,4R,5R)-5-(4-amino-5-ethynyl-7H-pyrrolo[2,3-d]pyrimidin-7-yl)-4-ethynyl-2-fluoro-2-(hydroxymethyl)tetrahydrofuran-3,4-diol

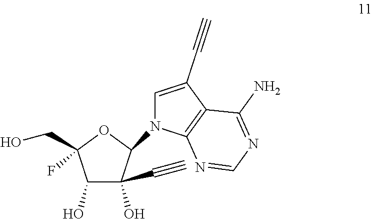 [SMILES: C#Cc1cn([C@@H]2O[C@](F)(CO)[C@@H](O)[C@]2(O)C#C)c2ncnc(N)c12]

Compound 11 can be prepared using the synthetic routes provided herein as examples and a starting point. Further information for preparing compound 11 is provided in PCT Publication No. WO 2014/100505 and U.S. Publication Nos. 2015/0011497 and 2015/0105341, which are each incorporated by reference in their entireties. Those skilled in the art will be able to recognize modifications of the disclosed syntheses and to devise routes based on the disclosures herein.

### Example 9

### Compound 12: (2R,3R,4R,5R)-2-(6-amino-9H-purin-9-yl)-5-(hydroxymethyl)-3-(propa-1,2-dien-1-yl)tetrahydrofuran-3,4-diol

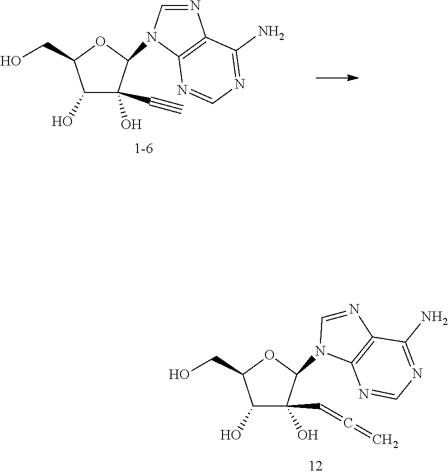 [SMILES: C#C[C@@]1(O)[C@H](O)[C@@H](CO)O[C@H]1n1cnc2c(N)ncnc21 ; C=C=C[C@@]1(O)[C@H](O)[C@@H](CO)O[C@H]1n1cnc2c(N)ncnc21]

To a solution of compound 1-2 ((2R,3R,4R,5R)-2-(6-amino-9H-purin-9-yl)-3-ethynyl-5-(hydroxymethyl)tetrahydrofuran-3,4-diol, 450 mg, 1.55 mmol) in dioxane (5.0 mL) was added CuBr (222.35 mg, 1.55 mmol), i-Pr₂NH (156 mg, 1.55 mmol, 1.20 eq.) and HCHO (188 mg, 2.3 mmol, 1.50 eq.). The mixture was stirred at 120° C. under microwave irradiation for 35 mins. The mixture was concentrated under reduced pressure. The residue was purified by prep-HPLC (column: Xtimate C18 150*25 mm*5 um; mobile phase: [water (0.225% FA)-ACN]; B %: 0%-10%, 11.5 min and lyophilized to give compound 12 (50 mg, 10.56%) as white solid. ¹H NMR (400 MHz, DMSO-d₆), δ=8.39 (s, 1H), 8.12 (s, 1H), 7.24 (s, 2H), 6.03 (s, 1H), 5.63 (s, 1H), 5.25 (s, 2H), 4.81-4.77 (m, 1H), 4.72-4.68 (m, 1H), 4.44 (dd, J=6.7, 11.4 Hz, 1H), 4.37 (d, J=9.0 Hz, 1H), 3.93 (d, J=9.0 Hz, 1H), 3.84 (d, J=11.8 Hz, 1H), 3.71-3.68 (m, 1H). ESI-LCMS: m/z 306.1 [M+H]⁺.

### Example 10

### Compound 13: (2S,3S,4R,5R)-5-(6-amino-9H-purin-9-yl)-2-fluoro-2-(hydroxymethyl)-4-methyltetrahydrofuran-3,4-diol

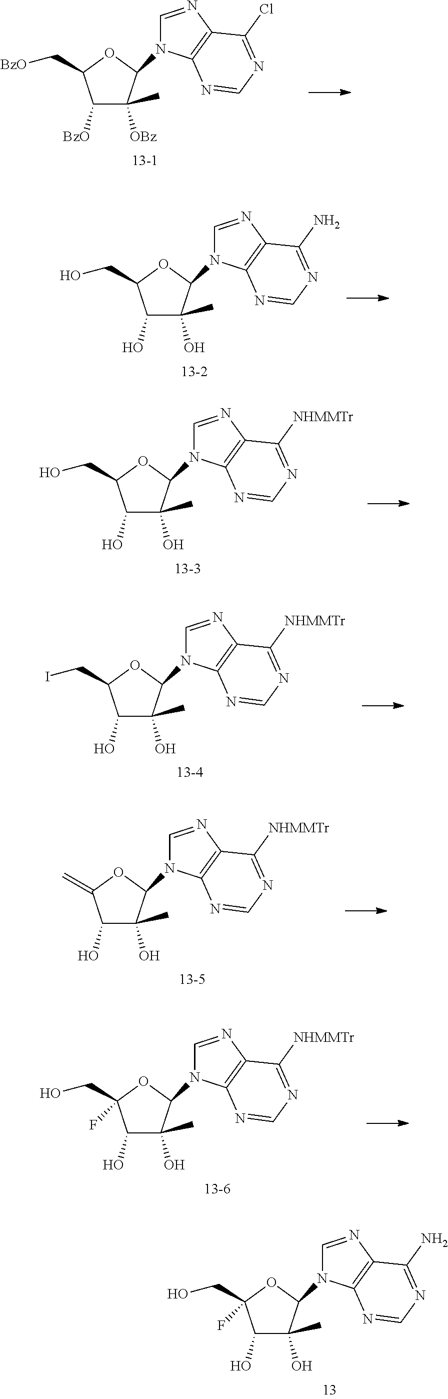

To a solution of Intermediate 2 ((3R,4R,5R)-5-((benzoyloxy)methyl)-3-methyltetrahydrofuran-2,3,4-triyltribenzoate, 33 g, 54.0 mmol) and 6-chloro-9H-purine (9.7 g, 62.1 mmol) in ACN (300 mL) was added DBU (25.9 g, 170.1 mmol) at 0° C. To this solution was added TMSOTf (50.4 g, 224.8 mmol) at 0° C. The solution was stirred for 15 mins at 0° C. and then 5 h at 65° C. The solution was diluted with of dichloromethane (DCM, 2000 mL), washed with NaHCO₃ (aq., 2×1000 mL). The resulting solution was dried over anhydrous Na₂SO₄, filtered and concentrated under reduced pressure. The residue was applied onto a silica gel column with EA/PE (1:2). Compound 13-5 was obtained ((2R,3R,4R,5R)-5-((benzoyloxy)methyl)-2-(6-chloro-9H-purin-9-yl)-3-methyltetrahydrofuran-3,4-diyldibenzoate, 33 g, 95%) as a yellow solid. ESI-MS: m/z 613 [M+H]⁺.

To a solution of compound 13-5 (40 g, 62 mmol) in dioxane (50 mL) was added ammonia (30%, 150 mL). The solution was stirred for 16 h at 110° C. in sealed tube. The solution was cooled to R.T., the mixture was concentrated under reduced pressure, washed with EA (2×400 mL). Compound 13-6 was obtained ((2R,3R,4R,5R)-2-(6-amino-9H-purin-9-yl)-5-(hydroxymethyl)-3-methyltetrahydrofuran-3,4-diol, crude, 16 g) as a white solid. ESI-MS: m/z 282 [M+H]⁺.

To a solution of compound 13-6 (8 g, 27.0 mmol) in pyridine (160 mL) was added trimethylchlorosilane (30.8 g, 283.5 mmol) at 0° C. The solution was stirred for 5 h at R.T. To this solution was added 4-methoxytriphenylmethyl chloride (26.3 g, 84.3 mmol). The solution was stirred for 16 h at 40° C. and then ammonia (30%, 40 mL) and tetrabutylammonium fluoride (1 M in THF, 40 mL) were added. The solution was stirred for 2 h at R.T., diluted with EA (1000 mL) and washed with water (2×500 mL). The solution was dried over anhydrous Na₂SO₄, filtered and concentrated under reduced pressure. The residue was applied onto a silica gel column with DCM/MeOH (50:1). Compound 13-7 was obtained ((2R,3R,4R,5R)-5-(hydroxymethyl)-2-(6-(((4-methoxyphenyl)diphenylmethyl)amino)-9H-purin-9-yl)-3-methyltetrahydrofuran-3,4-diol, 11 g, 70%) as a white solid. ESI-MS: m/z 554 [M+H]⁺.

To a solution of compound 13-7 (10 g, 18.1 mmol) and Ph₃P (7.1 g, 27.09 mmol) and imidazole (2.4 mg, 0.04 mmol) in pyridine:THF (2:5, 140 mL) at 0° C. was added iodine (6 g in THF (40 mL), 23.5 mmol). The solution was stirred for 2 h at R.T. and then concentrated under reduced pressure. The residue was applied onto a silica gel column with DCM/MeOH (70:1). Compound 13-8 was obtained ((2R,3R,4R,5S)-5-(iodomethyl)-2-(6-(((4-methoxyphenyl)diphenylmethyl)amino)-9H-purin-9H-yl)-3-methyltetrahydrofuran-3,4-diol, 3.2 g, 24%) as a white solid. ESI-MS: m/z 664 [M+H]⁺.

A solution of compound 13-8 (3 g, 4.5 mmol) in 5% NaOMe in MeOH (30 mL) was stirred for 16 h at 40° C. The mixture was concentrated under reduced pressure. The residue was applied onto a silica gel column with DCM/MeOH (50:1). Compound 13-9 was obtained ((2R,3R,4S)-2-(6-(((4-methoxyphenyl)diphenylmethyl)amino)-9H-purin-9-yl)-3-methyl-5-methylenetetrahydrofuran-3,4-diol, 1.7 g, 63%) as a white solid. ESI-MS: m/z 536 [M+H]⁺.

To a solution of compound 13-9 (1 g, 1.8 mmol) in DCM (8 mL) was added a solution of 3-chloroperoxybenzoic acid (70%, 690 mg, 4.0 mmol) in DCM (2 mL) at 0° C. To this solution was added TEA●3HF (902 mg, 5.6 mmol) at 0° C. The solution was stirred for 1 h at 0° C. and then concentrated under reduced pressure. The residue was applied onto a silica gel column with DCM/MeOH (40:1). Compound 13-10 was obtained ((2S,3 S,4R,5R)-2-fluoro-2-(hydroxymethyl)-5-(6-(((4-methoxyphenyl)diphenylmethyl)amino)-9H-purin-9-yl)-4-methyltetrahydrofuran-3,4-diol, 210 mg, 17%) as a white solid. ESI-MS: m/z 572 [M+H]⁺.

To a solution of compound 13-10 (500 mg, 0.87 mmol) in dioxane (5 mL) was added 5% trifluoroacetic acid (10 mL). The solution was stirred for 2 h at R.T. The pH value of the solution was adjusted to 8 with ammonia (30%) and then concentrated under reduced pressure. The crude product (500 mg) was purified by Prep-HPLC with the following conditions: Column, Atlantis Prep T3 OBD Column, 19*250 mm 10 u; mobile phase, waters and ACN (3.0% ACN up to 14.0% in 12 min); Detector, uv 254 nm. Compound 13 was obtained ((2S,3S,4R,5R)-5-(6-amino-9H-purin-9-yl)-2-fluoro-2-(hydroxymethyl)-4-methyltetrahydrofuran-3,4-diol, 86.5 mg, 31%) as a white solid. ESI-MS: m/z 300 [M+H]⁺.

### Example 11

### Compound 14: (2S,3S,4R,5R)-5-(6-amino-9H-purin-9-yl)-2,4-difluoro-2-(hydroxymethyl)-4-methyltetrahydrofuran-3-ol

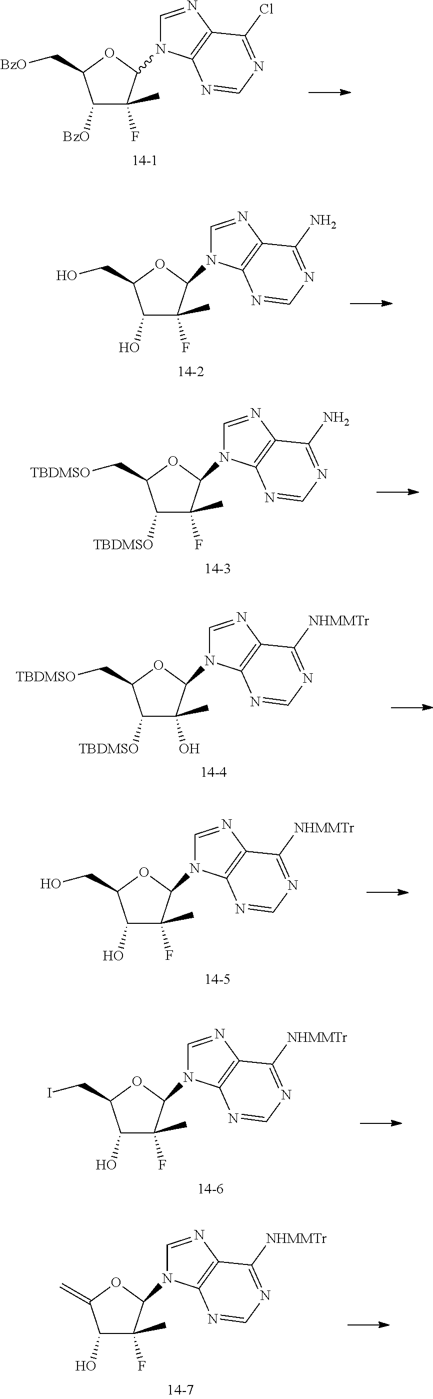

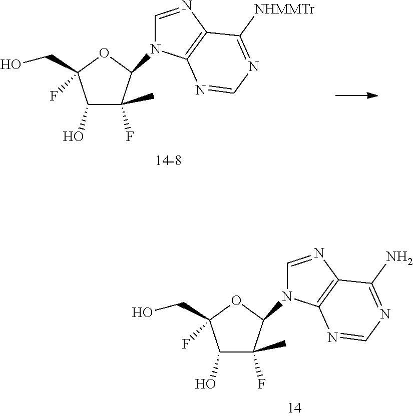

To a solution of Intermediate 3 ((2R,3R,4R)-3-(benzoyloxy)-4-fluoro-5-hydroxy-4-methyltetrahydrofuran-2-yl)methylbenzoate, 40 g, 106.9 mmol α/β=1/3), 6-chloro-9H-purine (24.8 g, 160.5 mmol) and Ph₃P (40 g, 152.5 mmol) in THF (400 mL) was added DEAD (37.2 g, 213.6 mmol) at 0° C. The resulting solution was stirred for 6 h at R.T., then concentrated under reduced pressure. The residue was applied onto a silica gel column with EA/PE (1:5). This resulted in 42 g (77%, α/β=1/1) of compound 14-1 (((2R,3R,4R)-3-(benzoyloxy)-5-(6-chloro-9H-purin-9-yl)-4-fluoro-4-methyltetrahydrofuran-2-yl)methyl benzoate)as yellow oil. ESI-MS: m/z 511 [M+H]⁺.

To a solution of compound 14-1 (10 g, 19.6 mmol, α/β=1/1) in dioxane (30 mL) was added ammonia (30%, 100 mL). The resulting solution was stirred for 16 h at 110° C. in sealed tube. The solution was cooled to R.T., the resulting mixture was concentrated under reduced pressure. The residue was applied onto a silica gel column with DCM/MeOH (10:1). This resulted in 2.1 g (38%) of compound 14-2 ((2R,3R,4R,5R)-5-(6-amino-9H-purin-9-yl)-4-fluoro-2-(hydroxymethyl)-4-methyltetrahydrofuran-3-ol) as yellow solid. ESI-MS: m/z 284 [M+H]⁺.

To a solution of compound 14-2 (100 mg, 0.35 mmol) and imidazole (144 mg, 2.1 mmol) in DMF (3 mL) was added tert-butyldimethylsilyl chloride (160 mg, 1.1 mmol) at 25° C. The resulting solution was stirred for 16 h at 60° C., then quenched by the addition of 100 mL of NaHCO₃ solution. The resulting solution was extracted with 2×100 mL of DCM and the organic layers combined. The resulting solution was dried over anhydrous Na₂SO₄, filtered and concentrated under reduced pressure. The residue was applied onto a silica gel column with PE/EA (1:1). This resulted in 157 mg (87%) of compound 14-3 (9-((2R,3R, 4R,5R)-4-((tert-butyldimethylsilyl)oxy)-5-(((tert-butyldimethylsilyl)oxy)methyl)-3-fluoro-3-methyltetrahydrofuran-2-yl)-9H-purin-6-amine) as yellow oil. ESI-MS: m/z 512 [M+H]⁺.

To a solution of compound 14-3 (200 mg, 0.39 mmol) and DMAP (9.5 mg, 0.08 mmol) in pyridine (3 mL) was added 4-methoxytriphenylmethyl chloride (241 mg, 0.8 mmol) at 25° C. The resulting solution was stirred for 48 h at 60° C., then quenched by the addition of 100 mL of NaHCO₃ solution. The resulting solution was extracted with 2×100 mL of DCM and the resulting solution was dried over anhydrous Na₂SO₄, filtered and concentrated under reduced pressure. This resulted in 400 mg crude of compound 14-4 (9-((2R,3R,4R,5R)-4-((tert-butyldimethylsilyl)oxy)-5-(((tert-butyldimethylsilyl)oxy)methyl)-3-fluoro-3-methyltetrahydrofuran-2-yl)-N-((4-methoxyphenyl)diphenylmethyl)-9H-purin-6-amine) as yellow oil. ESI-MS: m/z 784 [M+H]⁺.

To a solution of compound 14-4 (3 g, 3.8 mmol) in DCM (30 mL) was added tetrabutylammonium fluoride (23 mL, 1M in THF). The resulting solution was stirred for 3 h at R.T. The resulting mixture was concentrated under reduced pressure. The residue was applied onto a silica gel column with EA/PE (1:1). This resulted in 1.5 g (71%) of compound 14-5 ((2R,3R,4R,5R)-4-fluoro-2-(hydroxymethyl)-5-(6-(((4-methoxyphenyl)diphenylmethyl)amino)-9H-purin-9-yl)-4-methyltetrahydrofuran-3-ol) as yellow oil. ESI-MS: m/z 556 [M+H]⁺.

To a solution of compound 14-5 (1 g, 1.8 mmol), Ph₃P (1.89 g, 7.2 mmol) and imidazole (490 mg, 7.2 mmol) in THF (20 mL) was added iodine (1.37 g, 5.4 mmol) at 25° C. The resulting solution was stirred for 24 h at R.T., then concentrated under reduced pressure. The residue was applied onto a silica gel column with EA/PE (1:1). This resulted in 1.12 g (94%) of compound 14-6 ((2S,3R,4R,5R)-4-fluoro-2-(iodomethyl)-5-(6-(((4-methoxyphenyl)diphenylmethyl)amino)-9H-purin-9-yl)-4-methyltetrahydrofuran-3-ol) as a yellow solid. ESI-MS: m/z 666 [M+H]⁺.

To a solution of compound 14-6 (1.1 g, 1.65 mmol) in 15% NaOMe in methanol (10 mL) was stirred for 16 h at R.T. The resulting mixture was concentrated under reduced pressure. The residue was applied onto a silica gel column with DCM/MeOH (20:1). This resulted in 500 mg (56%) of compound 14-7 ((3R,4R,5R)-4-fluoro-5-(6-(((4-methoxyphenyl)diphenylmethyl)amino)-9H-purin-9-yl)-4-methyl-2-methylenetetrahydrofuran-3-ol) as a white solid. ESI-MS: m/z 538 [M+H]⁺.

To a solution of compound 14-7 (45 mg, 0.08 mmol) in DCE (1 mL) was added 3-chloroperoxybenzoic acid (70%, 41 mg, 0.17 mmol) and TEA·3HF (67 mg, 0.4 mmol) at 0° C. The resulting solution was stirred for 1 h at 0° C. and then concentrated under reduced pressure. The residue was applied onto a silica gel column with DCM/MeOH (20:1). This resulted in 14 mg (29%) of compound 14-8 ((2S,3S,4R,5R)-2,4-difluoro-2-(hydroxymethyl)-5-(6-(((4-methoxyphenyl)diphenylmethyl)amino)-9H-purin-9-yl)-4-methyltetrahydrofuran-3-ol) as a white solid. ESI-MS: m/z 574 [M+H]⁺.

To a solution of compound 14-8 (230 mg, 0.4 mmol) in 1,4-dioxane (0.5 mL) was added 5% TFA (1 mL). The resulting solution was stirred for 3 h at R.T. The pH value of the solution was adjusted to 7 with ammonia (30%) and then concentrated under reduced pressure. The crude product (230 mg) was purified by Prep-HPLC with the following conditions: Column, xBridge C18, 19 mm*250 mm, 5 μm; mobile phase, A: Water, mobile phase B: ACN (hold 3.0% ACN in 10 min); Detector, UV 254 nm. This resulted in 61.6 mg (51%) of compound 14 ((2S,3S,4R,5R)-5-(6-amino-9H-purin-9-yl)-2,4-difluoro-2-(hydroxymethyl)-4-methyltetrahydrofuran-3-ol) as a white solid. ESI-MS: m/z 302 [M+H]⁺. ¹H-NMR (300 MHz, CD₃OD): δ ppm 8.36 (s, 1H), 8.19 (s, 1H), 6.55 (d, J=16.5 Hz, 1H), 4.72 (m, 1H), 3.81 (m, 2H), 1.23 (d, J=14.7 Hz, 3H). ¹⁹F-NMR (300 MHz, CD₃OD): δ ppm −125.4, −160.2.

### Example 12

### Compound 15: (2S,3S,4R,5R)-5-(2,6-diamino-9H-purin-9-yl)-2,4-difluoro-2-(hydroxymethyl)-4-methyltetrahydrofuran-3-ol

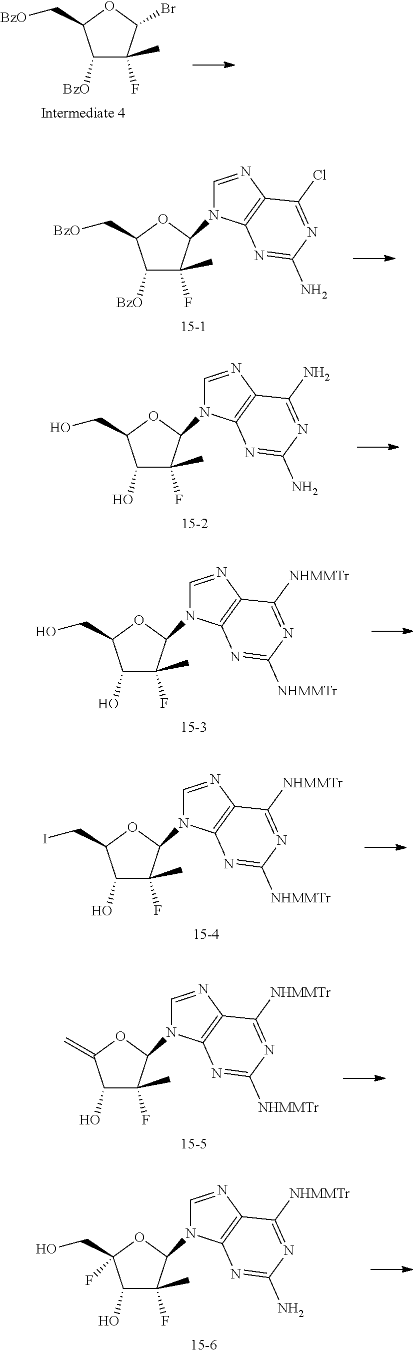

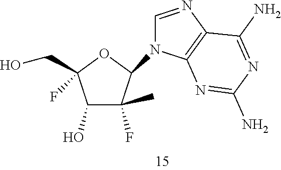 [SMILES: C[C@]1(F)[C@H](n2cnc3c(N)nc(N)nc32)O[C@](F)(CO)[C@H]1O]

To a suspension of 2-amine-6-chloro-9H-purine (1.45 g, 8.6 mmol) in t-BuOH (15 mL) was added t-BuOK (880 mg, 7.8 mmol) and stirred for 30 min. To this was added a solution of Intermediate 4 (((2R,3R,4R,5R)-3-(benzoyloxy)-5-bromo-4-fluoro-4-methyltetrahydrofuran-2-yl)methylbenzoate, 1.5 g, 3.4 mmol) in ACN (20 mL). The resulting solution was stirred for 16 h at 50° C. The solution was cooled to R.T., the pH was adjusted to 7 with AcOH, then diluted with 100 mL of EA, washed with 2×50 mL of water. The resulting solution was dried over anhydrous Na₂SO₄, filtered and concentrated under reduced pressure. The residue was applied onto a silica gel column with DCM/MeOH (50:1). This resulted in 1.26 g (70%) of compound 15-1 ((2S,3S,4R,5R)-5-(2,6-diamino-9H-purin-9-yl)-2,4-difluoro-2-(hydroxymethyl)-4-methyltetrahydrofuran-3-ol) as a yellow solid. ESI-MS: m/z 526 [M+H]⁺.

To a solution of compound 15-1 (3 g, 5.7 mmol) in 1,4-dioxane (5 mL) was added ammonia (30%, 15 mL). The resulting solution was stirred for 16 h at 110° C. in sealed tube. The solution was cooled to R.T., the resulting mixture was concentrated under reduced pressure. After re-crystallization from MeOH/EA, this resulted in 1.7 g (99%) of compound 15-2 ((2R,3R,4R,5R)-5-(2,6-diamino-9H-purin-9-yl)-4-fluoro-2-(hydroxymethyl)-4-methyltetrahydrofuran-3-ol) as a yellow solid. ESI-MS: m/z 299 [M+H]⁺.

To a solution of compound 15-2 (200 mg, 0.67 mmol) in pyridine (3 mL) was added trimethylchlorosilane (579 mg, 5.3 mmol) and stirred for 6 h at 30° C., then added 4-methoxytriphenylmethyl chloride (826 mg, 2.7 mmol) and stirred for 16 h at 40° C. To this was added ammonia (30%, 2 mL) and tetrabutylammonium fluoride (1 M in THF, 2 mL), stirred for 4 h. The resulting solution was extracted with 3×10 mL of EA. The resulting solution was dried over anhydrous Na₂SO₄, filtered and concentrated under reduced pressure. The residue was applied onto a silica gel column with DCM/MeOH (15:1). This resulted in 214.2 mg (38%) of compound 15-3 ((2R,3R,4R,5R)-5-(2,6-bis(((4-methoxyphenyl)diphenylmethyl)amino)-9H-purin-9-yl)-4-fluoro-2-(hydroxymethyl)-4-methyltetrahydrofuran-3-ol) as a yellow solid. ESI-MS: m/z 843 [M+H]⁺.

To a solution of compound 15-3 (1.5 g, 1.8 mmol) and Ph₃P (1.165 g, 4.45 mmol) and imidazole (298 mg, 4.4 mmol) in THF (15 mL) was added iodine (0.676 g, 2.7 mmol) at 0° C. The resulting solution was stirred for 2 h at 0° C. and then quenched by the addition of 50 mL of Na₂S₂O₃ solution. The resulting solution was extracted with 3×50 mL of EA. The resulting solution was dried over anhydrous Na₂SO₄, filtered and concentrated under reduced pressure. The residue was applied onto a silica gel column with EA/PE (3:5). This resulted in 197.6 mg (12%) of compound 15-4 ((2S,3R,4R,5R)-5-(2,6-bis(((4-methoxyphenyl)diphenylmethyl)amino)-9H-purin-9-yl)-4-fluoro-2-(iodomethyl)-4-methyltetrahydrofuran-3-ol) as a yellow solid. ESI-MS: m/z 953 [M+H]⁺.

A solution of compound 15-4 (1 g, 1.05 mmol) in 3% NaOMe in methanol (10 mL) was stirred for 2 h at 60° C. The solution was cooled to R.T., the pH of the solution was adjusted to 7 with AcOH. The resulting solution was concentrated under vacuum. The residue was applied onto a silica gel column with EA/PE (1:1). This resulted in 423 mg (49%) of compound 15-5 ((3R,4R,5R)-5-(2,6-bis(((4-methoxyphenyl)diphenylmethyl)amino)-9H-purin-9-yl)-4-fluoro-4-methyl-2-methylenetetrahydrofuran-3-ol) as a yellow solid. ESI-MS: m/z 847 [M+H]⁺.

To a solution of compound 15-5 (1.2 g, 1.45 mmol) in DCM (20 mL) was added TEA·3HF (1.17 g, 7.3 mmol) and 3-chloroperoxybenzoic acid (710 mg, 4.1 mmol) at 0° C. The resulting solution was stirred for 2 h at 0° C. and then quenched by the addition of 50 mL of NaHCO₃ solution, extracted with 3×50 mL of EA. The resulting solution was dried over anhydrous Na₂SO₄, filtered and concentrated under reduced pressure. The residue was applied onto a silica gel column with DCM/MeOH (10:1). This resulted in 375 mg (44%) of compound 15-6 ((2S,3S,4R,5R)-5-(2-amino-6-(((4-methoxyphenyl)diphenylmethyl)amino)-9H-purin-9-yl)-2,4-difluoro-2-(hydroxymethyl)-4-methyltetrahydrofuran-3-ol) as a yellow solid. ESI-MS: m/z 589 [M+H]₊.

To a solution of compound 15-6 (200 mg, 0.34 mmol) in 1,4-dioxane (2 mL) was added 5% TFA (6 mL). The resulting solution was stirred for 2 h at R.T. The pH of the solution was adjusted to 7 with ammonia (30%) and then concentrated under reduced pressure. The crude product (150 mg) was purified by Prep-HPLC with the following conditions: Column, XBridge Prep C18 OBD Column, 19 mm*250 mm, 5 um; mobile phase, Waters (10 mmol/L NH₄HCO₃) and ACN (3.0% ACN up to 15.0% in 15 min); Detector, uv 254 nm. This resulted in 79.8 mg (74%) of compound 15 ((2S,3S,4R,5R)-5-(2,6-diamino-9H-purin-9-yl)-2,4-difluoro-2-(hydroxymethyl)-4-methyltetrahydrofuran-3-ol) as a white solid. ESI-MS: m/z 317 [M+H]⁻. ¹H-NMR (400 MHz, CD₃OD): δ ppm 8.02 (s, 1H), 6.44 (d, J=16.9 Hz, 1H), 4.76˜4.65 (m, 1H), 3.91˜3.79 (m, 2H), 1.25 (d, J=22.3 Hz, 3H). ¹⁹F-NMR (400 MHz, CD₃OD): δ ppm −125.22, −160.15.

### Example 13

### Compound 16: (2R,3R,4R,5R)-2-(6-amino-2-fluoro-9H-yl)-purin-9-yl)-5-(hydroxymethyl)-3-methyltetrahydrofuran-3,4-diol

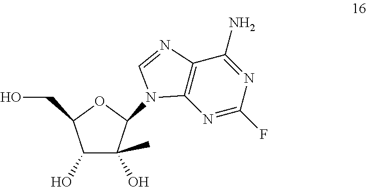 [SMILES: C[C@@]1(O)[C@H](O)[C@@H](CO)O[C@H]1n1cnc2c(N)nc(F)nc21]

Compound 16 can be prepared using the synthetic routes provided herein as examples and a starting point. Further information for preparing compound 16 is provided in U.S. Publication Nos. 2013/0165400, 2015/0011497 and 2015/0105341, which are each incorporated by reference in their entireties. Those skilled in the art will be able to recognize modifications of the disclosed syntheses and to devise routes based on the disclosures herein.

### Example 14

### Triphosphates

The triphosphates summarized in Table 3 below were prepared from the corresponding nucleosides in accordance with the following general procedure: Dry nucleoside (0.05 mmol) was dissolved in dry PO(OMe)₃ (0.7 mL). N-Methylimidazole (0.009 mL, 0.11 mmol) was added followed by POCl₃ (0.009 mL, 0.11 mmol) and the mixture was kept at R.T., for 20-40 mins. The reaction was controlled by LCMS and monitored by the appearance of corresponding nucleoside 5′-monophosphate. After completion of the reaction, tetrabutylammonium salt of pyrophosphate (150 mg) was added, followed by DMF (0.5 mL) to get a homogeneous solution. After 1.5 h at ambient temperature, the reaction was diluted with water (10 mL) and loaded on the column HiLoad 16/10 with Q Sepharose High Performance. Separation was done in a linear gradient of NaCl from 0 to 1N in 50 mM TRIS-buffer (pH 7.5). Triphosphate was eluted at 75-80% B. Corresponding fractions were concentrated. Desalting was achieved by RP HPLC on Synergy 4 micron Hydro-RP column (Phenominex). A linear gradient of MeOH from 0 to 30% in 50 mM triethylammonium acetate buffer (pH 7.5) was used for elution. The corresponding fractions were combined, concentrated and lyophilized (3×) to remove excess of buffer.

TABLE 3

| | | MS | | | |
|---|---|---|---|---|---|
| No. | Structure | [M − 1] | P(α) | P(β) | P(γ) |
| 17 | 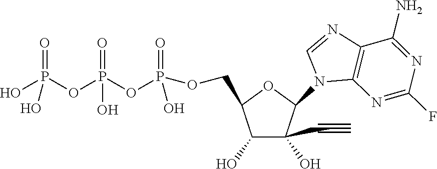 [SMILES: C#C[C@@]1(O)[C@H](O)[C@@H](COP(=O)(O)OP(=O)(O)OP(=O)(O)O)O[C@H]1n1cnc2c(N)nc(F)nc21] | 547.8 | −6.65 (d) | −22.21(t) | −11.24 (d) |
| 18 | 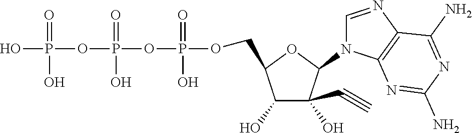 [SMILES: C#C[C@@]1(O)[C@H](O)[C@@H](COP(=O)(O)OP(=O)(O)OP(=O)(O)O)O[C@H]1n1cnc2c(N)nc(N)nc21] | 544.6 | −10.78 (d) | −23.19(t) | −11.41 (d) |
| 19 | 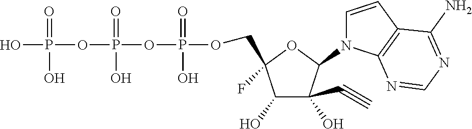 [SMILES: C#C[C@]1(O)[C@H](n2ccc3c(N)ncnc32)O[C@](F)(COP(=O)(O)OP(=O)(O)OP(=O)(O)O)[C@H]1O] | 547.1 | −7.98 (d) | −22.47(t) | −10.78 (d) |
| 20 | 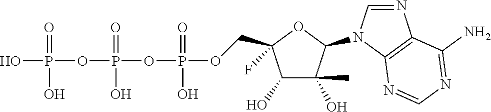 [SMILES: C[C@]1(O)[C@H](n2cnc3c(N)ncnc32)O[C@](F)(COP(=O)(O)OP(=O)(O)OP(=O)(O)O)[C@H]1O] | 538.4 | −9.20 (br.s) | −22.50(t) | −12.04 (br.s) |
| 21 | 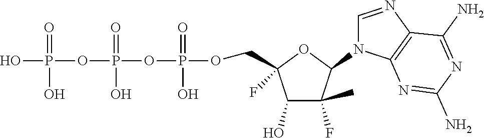 [SMILES: C[C@]1(F)[C@H](n2cnc3c(N)nc(N)nc32)O[C@](F)(COP(=O)(O)OP(=O)(O)OP(=O)(O)O)[C@H]1O] | 554.5 | −10.72 (d) | −23.08(t) | −12.09 (d) |
| 22 | 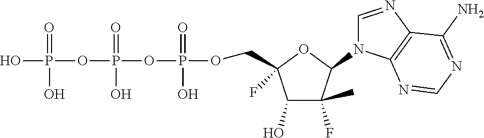 [SMILES: C[C@]1(F)[C@H](n2cnc3c(N)ncnc32)O[C@](F)(COP(=O)(O)OP(=O)(O)OP(=O)(O)O)[C@H]1O] | 539.8 | −6.44 (d) | −22.37(t) | −12.26 (d) |
| 23 | 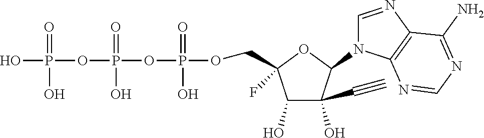 [SMILES: C#C[C@]1(O)[C@H](n2cnc3c(N)ncnc32)O[C@](F)(COP(=O)(O)OP(=O)(O)OP(=O)(O)O)[C@H]1O] | 547.5 | −10.99 (br.s) | −23.21(t) | −12.27(d) |
| 24 | 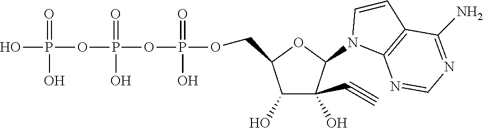 [SMILES: C#C[C@@]1(O)[C@H](O)[C@@H](COP(=O)(O)OP(=O)(O)OP(=O)(O)O)O[C@H]1n1ccc2c(N)ncnc21] | 528.8 | −6.44 (d) | −22.45(t) | −11.31 (d) |
| 25 | 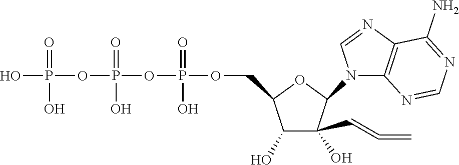 [SMILES: C=C=C[C@@]1(O)[C@H](O)[C@@H](COP(=O)(O)OP(=O)(O)OP(=O)(O)O)O[C@H]1n1cnc2c(N)ncnc21] | 544.3 | −10.96 (d) | −23.32(t) | −11.50(d) |
| 35 | 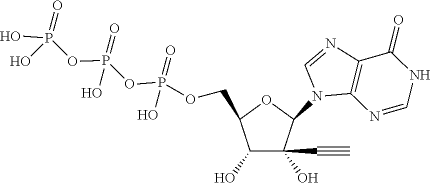 [SMILES: C#C[C@@]1(O)[C@H](O)[C@@H](COP(=O)(O)OP(=O)(O)OP(=O)(O)O)O[C@H]1n1cnc2c(=O)[nH]cnc21] | 531.1 | −11.03 (d) | −23.35(t) | −11.53 (d) |
| 45 | 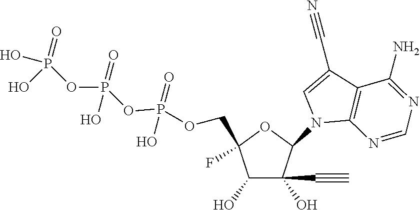 [SMILES: C#C[C@]1(O)[C@H](n2cc(C#N)c3c(N)ncnc32)O[C@](F)(COP(=O)(O)OP(=O)(O)OP(=O)(O)O)[C@H]1O] | 572.3 | −10.84 (d) | −23.20(t) | −12.28 (d) |
| 50 | 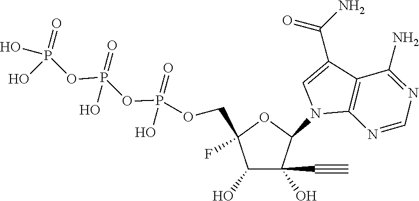 [SMILES: C#C[C@]1(O)[C@H](n2cc(C(N)=O)c3c(N)ncnc32)O[C@](F)(COP(=O)(O)OP(=O)(O)OP(=O)(O)O)[C@H]1O] | 590.8 | −10.87 (d) | −23.29(t) | −11.05 (d) |
| 51 | 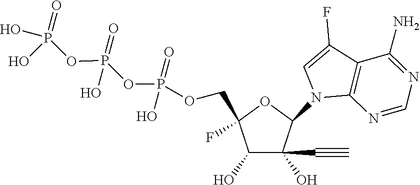 [SMILES: C#C[C@]1(O)[C@H](n2cc(F)c3c(N)ncnc32)O[C@](F)(COP(=O)(O)OP(=O)(O)OP(=O)(O)O)[C@H]1O] | 565.4 | −10.75 (d) | −23.15(t) | −12.32 (d) |
| 52 | 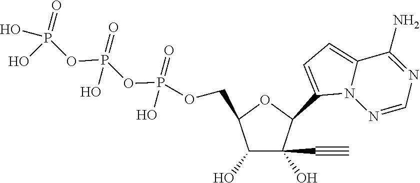 [SMILES: C#C[C@@]1(O)[C@H](O)[C@@H](COP(=O)(O)OP(=O)(O)OP(=O)(O)O)O[C@H]1c1ccc2c(N)ncnn12] | 529.2 | −9.44 (d) | −23.03(t) | −11.27 (d) |
| 53 | 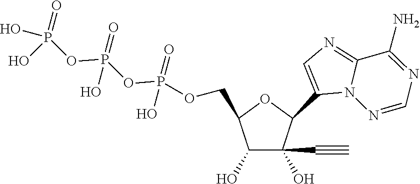 [SMILES: C#C[C@@]1(O)[C@H](O)[C@@H](COP(=O)(O)OP(=O)(O)OP(=O)(O)O)O[C@H]1c1cnc2c(N)ncnn12] | 530.3 | −10.99 (d) | −23.31(t) | −11.34 (d) |
| 54 | 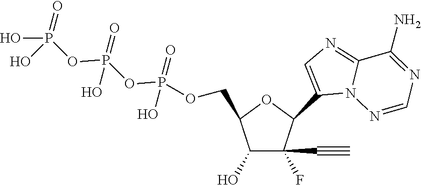 [SMILES: C#C[C@@]1(F)[C@H](O)[C@@H](COP(=O)(O)OP(=O)(O)OP(=O)(O)O)O[C@H]1c1cnc2c(N)ncnn12] | 532.3 | −11.01 (d) | −23.34(t) | −11.41 (d) |
| 55 | 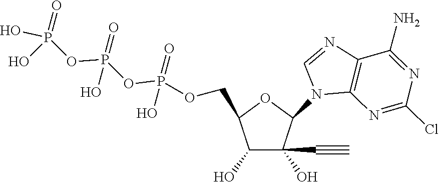 [SMILES: C#C[C@@]1(O)[C@H](O)[C@@H](COP(=O)(O)OP(=O)(O)OP(=O)(O)O)O[C@H]1n1cnc2c(N)nc(Cl)nc21] | 564.2 | −10.99 (d) | −23.36(t) | −11.52 (d) |
| 56 | 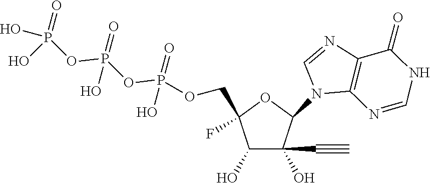 [SMILES: C#C[C@]1(O)[C@H](n2cnc3c(=O)[nH]cnc32)O[C@](F)(COP(=O)(O)OP(=O)(O)OP(=O)(O)O)[C@H]1O] | 549.4 | −6.82 (br.s) | −22.25(t) | −12.01 (d) |
| 57 | 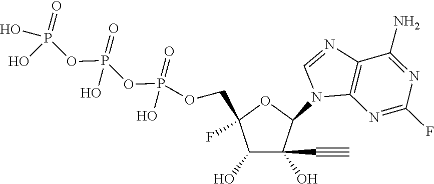 [SMILES: C#C[C@]1(O)[C@H](n2cnc3c(N)nc(F)nc32)O[C@](F)(COP(=O)(O)OP(=O)(O)OP(=O)(O)O)[C@H]1O] | 566.8 | −11.00 (d) | −23.28(t) | −12.31 (d) |
| 58 | 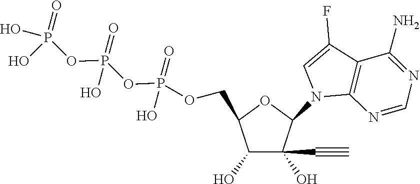 [SMILES: C#C[C@@]1(O)[C@H](O)[C@@H](COP(=O)(O)OP(=O)(O)OP(=O)(O)O)O[C@H]1n1cc(F)c2c(N)ncnc21] | 547.2 | −10.02 (d) | −23.18(t) | −11.49 (d) |

### Example 15

### Compound 26: (2S,3R,4R,5R)-2-(4-aminopyrrolo[2,1-f][1,2,4]triazin-7-yl)-3-ethynyl-5-(hydroxymethyl)tetrahydrofuran-3,4-diol

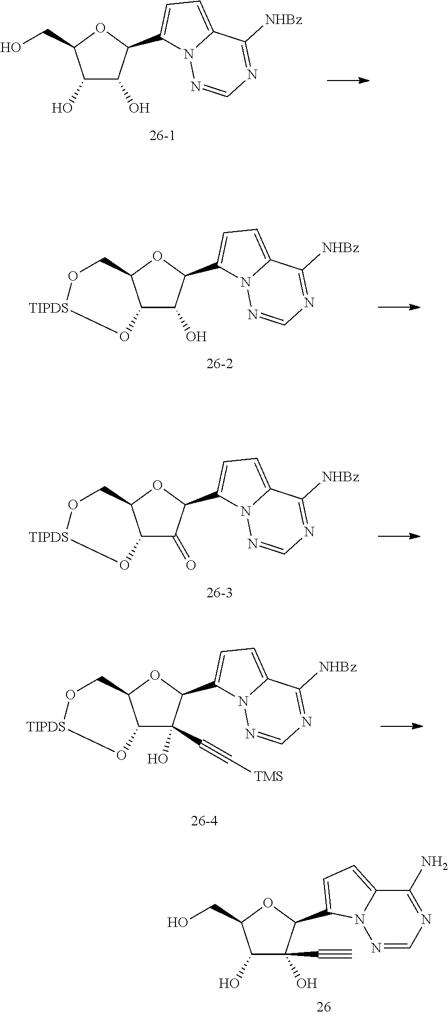

Compound 26-1 was prepared similarly to 15-1, using 7-bromopyrrolo[2,1-f][1,2,4]triazin-4-amine. To a solution of compound 26-1 ((2S,3R,4S,5R)-2-(4-(benzylamino)pyrrolo[2,1-f][1,2,4]triazin-7-yl)-5-(hydroxymethyl)tetrahydrofuran-3,4-diol, 1.82 g, 4.9 mmol) in pyridine (20 mL) was added chloro-[chloro(diisopropyl)silyl]oxy-diisopropyl-silane (1.63 g, 5.2 mmol, 1.64 mL). The reaction was stirred at 25° C. for 12 h. The reaction was quenched with saturated NH₄Cl (30 mL) and extracted with EA (50 mL). The organic layer was washed with brine (60 mL), dried over Na₂SO₄ and filtered. After concentrating under reduced pressure, the residue was applied onto a silica gel column with PE/EA (20:1 to 3:1) to give compound 26-2 ((6aR,8S,9S,9aS)-8-(4-(benzylamino)pyrrolo[2,1-f][1,2,4]triazin-7-yl)-2,2,4,4-tetraisopropyltetrahydro-6H-furo[3,2-f][1,3,5,2,4]trioxadisilocin-9-ol, 2.36 g, 3.8 mmol, 77.64%, 99% purity) as colorless oil. ¹H NMR (400 MHz, CDCl₃) δ=8.50-8.18 (m, 3H), 7.51-7.35 (m, 4H), 7.00-6.81 (m, 1H), 5.38-5.29 (m, 1H), 4.54 (s, 1H), 4.36 (s, 1H), 4.14-4.06 (m, 3H), 3.00 (s, 1H), 1.09-1.03 (m, 28H).

To a solution of compound 26-2 (2.30 g, 3.75 mmol) in ACN (25 mL) was added IBX (2.10 g, 7.5 mmol). The mixture was stirred at 90° C. for 2 h. The mixture was diluted with ACN (20 mL) and filtered. After concentrating under reduced pressure, the residue was applied onto a silica gel column with PE/EA (20:1 to 5:1) to give compound 26-3 ((6aR,8S,9aR)-8-(4-(benzylamino)pyrrolo[2,1-f][1,2,4]triazin-7-yl)-2,2,4,4-tetraisopropyldihydro-6H-furo[3,2-f][1,3,5,2,4]trioxadisilocin-9(8H)-one, 2.02 g, 3.3 mmol, 88%) as light yellow oil. LCMS: ESI-MS: m/z 611.0 [M+H]⁺.

To a solution of ethynyl(trimethyl)silane (963.54 mg, 9.8 mmol, 1.36 mL) in Et₂O (15.00 mL) was added n-BuLi (2.5 M, 3.92 mL) drop-wise at −78° C. The mixture was stirred at −78° C. for 1 h. A mixture solution of compound 26-3 (2.0 g, 3.3 mmol) in Et₂O (15 mL) was added drop-wise to the above solution at −78° C. and stirred at 0° C. for another 1 h. The reaction was quenched with saturated NaHCO₃ solution (40 mL) and extracted twice with EA (30 mL). The organic phase was washed with brine (60 mL), dried over anhydrous Na₂SO₄. After concentrating under reduced pressure, the residue was applied onto a silica gel column with PE/EA (30:1 to 5:1) to give compound 26-4 ((6aR,8S,9S,9aR)-8-(4-(benzylamino)pyrrolo[2,1-f][1,2,4]triazin-7-yl)-2,2,4,4-tetraisopropyl-9-((trimethylsilyl)ethynyl)tetrahydro-6H-furo[3,2-f][1,3,5,2,4]trioxadisilocin-9-ol, 220 mg, 285 μmol, 9%) as light yellow foam. LCMS: ESI-MS: m/z 709.1 [M+H]⁺.

To a solution of compound 26-4 (220 mg, 310 μmol) in MeOH (10.0 mL) was added NH₄F (230 mg, 6.2 mmol). The mixture was stirred at 80° C. for 11 h. NH₃.H₂O (194.2 mg, 1.55 mmol) was added into the above solution and kept stirring for another 1 h. After concentrating under reduced pressure, the residue was purified by Prep-HPLC (water (0.05% ammonia hydroxide v/v)-ACN) to give compound 26 ((2S,3R,4R,5R)-2-(4-aminopyrrolo[2,1-f][1,2,4]triazin-7-yl)-3-ethynyl-5-(hydroxymethyl)tetrahydrofuran-3,4-diol, 42.30 mg, 143.25 μmol, 46.17%, 98.3% purity) as white solid. ¹H NMR (400 MHz, MeOD) δ=7.76 (s, 1H), 6.85 (s, 2H), 5.60 (s, 1H), 4.27 (d, J=7.2 Hz, 1H), 3.89-3.98 (m, 2H), 3.78-3.81 (m, 1H), 2.57 (s, 1H). MS: m/z 291.11 [M+H]⁺.

### Example 16

### Compound 27: (2S,3R,4R,5R)-2-(4-aminoimidazo[2,1-f][1,2,4]triazin-7-yl)-3-ethynyl-5-(hydroxymethyl)tetrahydrofuran-3,4-diol

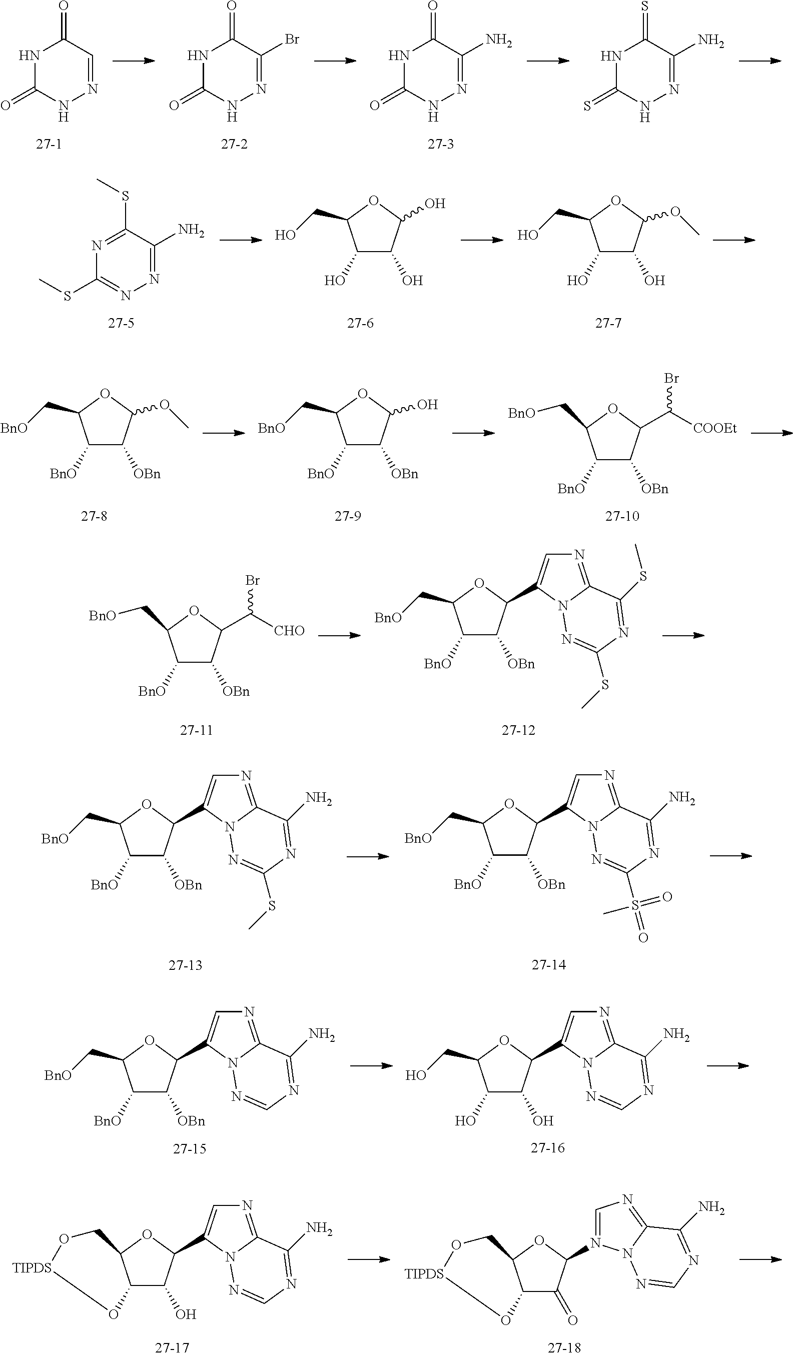

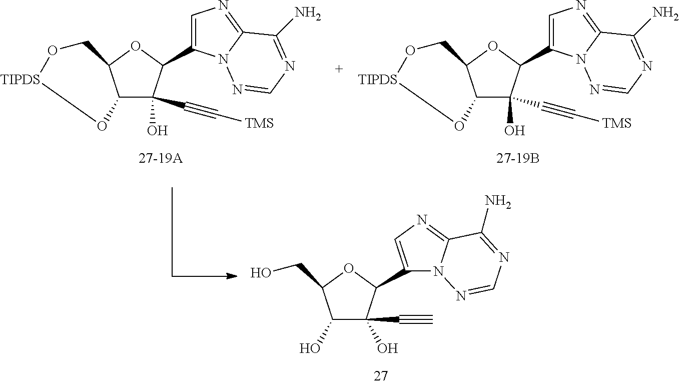

To a solution of compound 27-1 (1,2,4-triazine-3,5(2H,4H)-dione, 25.0 g, 221 mmol) in H₂O (350 mL) was added Br₂ (77.50 g, 485 mmol) drop-wise. The mixture was stirred at 25° C. for 24 h. The reaction was set up for 2 batches. The mixture was filtered to give a white solid. The solid was dried under reduced pressure with oil pump. Compound 27-2 (6-bromo-1,2,4-triazine-3,5(2H,4H)-dione, 40.0 g, 47.1%) was obtained as a white solid. ¹H NMR (400 MHz, DMSO-d₆) δ=12.55 (s, 1H), 12.29 (s, 1H).

Compound 27-2 (10.0 g, 52.1 mmol) in sealed tube was treated with Cu (331.03 mg, 5.2 mmol, 37 μL) and NH₃ (50.0 mL) and the reaction was stirred at 80° C. for 48 h. The reaction was set up for 4 batches. The mixture was cooled up to −40° C. and NH₃ (liquid) was volatilization. The crude was dissolved with hot H₂O (400 mL). The resulting solution was adjusted to pH=4 with conc. HCl solution. The resulting suspension was filtered, dissolved in dilute aq. NH₄OH and filtered again. The filtrate was acidified with conc. HCls until a precipitate formed and the suspension was filtered to give a white solid. Compound 27-3 (6-amino-1,2,4-triazine-3,5(2H,4H)-dione, 15.40 g, 120.2 mmol, 57.7%) was obtained as a white solid. ¹H NMR (400 MHz, DMSO-d₆) δ=11.72 (s, 1H), 10.87 (s, 1H), 5.94 (d, J=3.7 Hz, 2H).

To a solution of compound 27-3 (7.70 g, 60.1 mmol) in pyridine (500.0 mL) was added P₂S₃ (29.40 g, 132 mmol, 14.1 mL). The mixture was stirred at 130° C. for 7 h. The reaction was set up for 2 batches. Pyridine was removed under reduced pressure. The crude was dissolved in H₂O (500 mL). The suspension was stirred at 100° C. and then stand for 18 h. The solid was collected by filtration. The solid was dissolved in H₂O (300 mL). The resulting solution was adjusted to pH=10 by addition of NH₄OH solution, treated with norit and filtered to give the filtrate. The filtrate was then acidified with cone, HCl. After concentrating under reduced pressure, compound 27-4 (6-amino-1,2,4-triazine-3,5(2H,4H)-dithione, 10.0 g, 51.9%) was obtained as a brown solid. ¹H NMR (400 MHz, DMSO-d₆) δ=14.25 (s, 1H), 13.02 (s, 1H), 6.63 (s, 2H).

To a solution of compound 27-4 (5.20 g, 32.5 mmol) in DCM (400.0 mL) was added DIEA (25.17 g, 194.8 mmol, 34.0 mL) and MeI (13.40 g, 94.4 mmol, 5.9 mL). The mixture was stirred at 25° C. for 12 h. After concentrating under reduced pressure, the residue was applied onto a silica gel column with PE/EA (10:1 to 1:2). Compound 27-5 (3,5-bis(methylthio)-1,2,4-triazin-6-amine, 5.0 g, 26.6 mmol, 81.8%) was obtained as a yellow solid. ¹H NMR (400 MHz, CDCl₃) δ=4.65 (s, 2H), 2.60-2.61 (m, 6H).

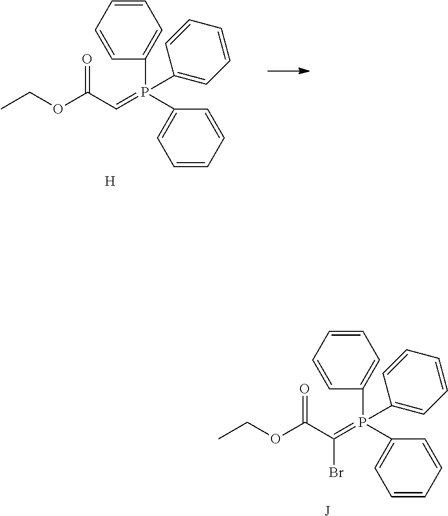

To a solution of compound H (ethyl2-(triphenyl-15-phosphanylidene)acetate, 25.0 g, 71.8 mmol) in DCM (200 mL) was added Br₂ (12.6 g, 78.9 mmol, 4.1 mL) in DCM (50 mL). The mixture was stirred at −40-20° C. for 12 h. The reaction was set up for 4 batches. The combined mixture was added DCM (100 mL) and water (100 mL). The resulting solution was washed with NaHCO₃ (aq., 2×200 mL) until the solution was neutralized and the organic phase over anhydrous Na₂SO₄ and concentrated in vacuum. The residue was recrystallized from acetone/n-hexane (2:1) (180 mL). The crystals were dried in vacuum. Compound J (ethyl2-bromo-2-(triphenyl-15-phosphanylidene)acetate, 102.0 g, 238.7 mmol, 83.2%) was obtained as a yellow solid. ¹H NMR (400 MHz, CDCl₃) δ=7.86-7.41 (m, 15H), 3.98 (q, J=7.2 Hz, 2H), 0.94 (t, J=7.2 Hz, 3H).

To a solution of compound 27-6 ((3R,4S,5R)-5-(hydroxymethyl)tetrahydrofuran-2,3,4-triol, 20.0 g, 133.2 mmol) in MeOH (150.0 mL) was added H₂SO₄. (2.40 g, 24 mmol). The mixture was stirred at 25° C. for 12 h. The mixture was diluted with MeOH (200 mL). The resulting solution was adjusted to pH=8 by adding Na₂CO₃ solid. After concentrating under reduced pressure, the residue was applied onto a silica gel column with DCM/MeOH (25:1 to 5:1) to give compound 27-7 ((2R,3S,4R)-2-(hydroxymethyl)-5-methoxytetrahydrofuran-3,4-diol, 32.40 g, 74.1%) as colorless oil.

To a solution of 27-7 (20.0 g, 121.8 mmol) in DMF (200 mL) was added NaH (17.1 g, 426.4 mmol) at 0° C. The mixture was stirred at 0° C. for 1 h. The resulting solution was treated with TBAI (4.50 g, 12.2 mmol) and BnBr (72.93 g, 426.4 mmol, 50.7 mL). The mixture was stirred at 25° C. for 11 h. The mixture was diluted with water (200 mL) and quenched with saturated NH₄Cl solution (100 mL). The resulting solution was extracted with EA (200 mL). The combined organic layers were washed twice with brine (200 mL) and dried over anhydrous Na₂SO₄. After concentrating under reduced pressure, the residue was applied onto a silica gel column with PE/EA (25:1 to 5:1) to give compound 27-8 ((2R,3R,4R)-3,4-bis(benzyloxy)-2-((benzyloxy)methyl)-5-methoxytetrahydrofuran, 37.20 g, 70%) as light yellow oil.

Compound 27-8 (20.0 g, 46.0 mmol) was dissolved in a mixture solution of TFA (56.0 mL) and H₂O (24.0 mL). The mixture was stirred at 25° C. for 12 h. The mixture was diluted with water (200 mL) and quenched with solid NaHCO₃ (80 g). The resulting solution was extracted with EA (300 mL). The organic layers were washed twice with brine (100 mL) and dried over anhydrous Na₂SO₄. After concentrating under reduced pressure, the residue was applied onto a silica gel column with PE/EA (25:1 to 5:1) to give compound 27-9 ((3R,4R,5R)-3,4-bis(benzyloxy)-5-((benzyloxy)methyl)tetrahydrofuran-2-ol, 37.8 g, 65.1%) as colorless oil. ESI-MS: m/z 443.1 [M+Na]⁺.

To a solution of compound 27-9 (10.0 g, 23.7 mmol) in toluene (100.0 mL) was added compound J (15.24 g, 35.7 mmol). The mixture was stirred at 110° C. for 8 h. The reaction was set up for 5 batches. The mixture was treated with DBU (60 drops) and stirred for 1 min. After concentrating under reduced pressure, the residue was applied onto a silica gel column with PE/EA (20:1 to 10:1). Compound 27-10 (ethyl2-((3R,4R,5R)-3,4-bis(benzyloxy)-5-((benzyloxy)methyl)tetrahydrofuran-2-yl)-2-bromoacetate, 45.0 g, 63.80%) was obtained as a light oil. LCMS: ESI-MS: m/z=591.1 [M+Na]⁺.

To a solution of compound 27-10 (12.50 g, 22 mmol) in toluene (125 mL) was added DIBAL-H (1 M, 43.90 mL). The mixture was stirred at −70° C. for 20 mins. The reaction was set up for 2 batches. The reaction was quenched by addition of MeOH (100 mL) and then diluted with EA (200 mL). After concentrating under reduced pressure, the residue was applied onto a silica gel column with PE/EA (15:1 to 3:1) to give compound 27-11 (2-((3R,4R,5R)-3,4-bis(benzyloxy)-5-((benzyloxy)methyl)tetrahydrofuran-2-yl)-2-bromoacetaldehyde, 20.0 g, 52%) as a light oil. ¹H NMR (400 MHz, CD₃Cl) δ=9.47-9.37 (m, 1H), 7.34-7.31 (m, 15H), 4.60-4.51 (m, 6H), 4.25-4.29(m, 2H), 4.17-4.08 (m, 1H), 4.06-4.00 (m, 1H), 3.99-3.93 (m, 1H), 3.56-3.47 (m, 2H).

To a solution of compound 27-11 (10.0 g, 19.0 mmol) in toluene (150 mL) was added 4A MS and compound 27-5 (3,5-bis(methylthio)-1,2,4-triazin-6-amine, 3.10 g, 16.5 mmol) in HMPA (50.0 mL). The mixture was stirred at 100° C. for 18 h. The reaction was set up for 2 batches. The mixture was concentrated under reduced pressure. The crude was dissolved in EA (200 mL) and H₂O (100 mL). The filtrate was collected and washed with brine (100 mL) and H₂O (100 mL) and dried over Na₂SO₄ (10 g), filtered and concentrated under reduced pressure. The residue was applied onto a silica gel column with PE/EA (5:1 to 3:1) to give compound 27-12 (7-((3S,4R,5R)-3,4-bis(benzyloxy)-5-((benzyloxy)methyl)tetrahydrofuran-2-yl)-2,4-bis(methylthio)imidazo[2,1-f][1,2,4]triazine, 8.90 g, 39.55%) as a brown oil. LCMS: ESI-MS: m/z=615.1 [M+H]⁺, 637.1 [M+Na]⁺.

To a solution of compound 27-12 (3.80 g, 6.2 mmol) in THF (10.0 mL) was added NH₃ (7 M in MeOH, 69.1 mL). The mixture was stirred at 60° C. for 24 h. The reaction was set up for 4 hatches. The mixture was concentrated under reduced pressure after excess NH₃ was volatized. The residue was applied onto a silica gel column with (PE/EA 5:1 to 0:1) to give 27-13 (7-((2S,3S,4R,5R)-3,4-bis(benzyloxy)-5-((benzyloxy)methyl)tetrahydrofuran-2-yl)-2-(methylthio)imidazo[2,1-f][1,2,4]triazin-4-amine, 9.80 g, 63.84%) as a brown foam. LCMS: ESI-MS: m/z=584.1 [M+H]⁻, 606.1 [M+Na]⁺.

To a solution of compound 27-13 (2.45 g, 4.2 mmol) in DCM (250 mL) was added m-CPBA (2.72 g, 12.6 mmol). The mixture was stirred at 0-25° C. for 18 h. The reaction was set up for 4 batches. The reaction was quenched by adding conc. NaHCO₃ and conc. Na₂S₂O₃ (v/v=200:200, mL) solution. The resulting mixture was extracted with DCM (200 mL). The organic layer was washed with brine (400 mL), dried over Na₂SO₄, filtered and concentrated under reduced pressure. After concentrating under reduced pressure, the residue was applied onto a silica gel column with PE/EA (15:100 to 0:100) PE/EA to give compound 27-14 (7-((2S,3S,4R,5R)-3,4-bis(benzyloxy)-5-((benzyloxy)methyl)tetrahydrofuran-2-yl)-2-(methylsulfonyl)imidazo[2,1-f][1,2,4]triazin-4-amine, 7.50 g, 72.5%) as a yellow foam. LCMS: ESI-MS: m/z=616.4 [M+H]⁺, 638.2 [M+Na]⁺.

To a solution of compound 27-14 (2.50 g, 4.1 mmol) in THF (100.0 mL) was added LiBHEt₃ (1 M, 162.40 mL) drop-wise at −70° C. The mixture was stirred at 18° C. for 2 h. The reaction was set up for 3 batches. The reaction was quenched with water (40 mL) and then extracted with EA (300 mL) and brine (300 mL). The combined organic layers were dried over anhydrous MgSO₄ and filtered. After concentrating under reduced pressure, the residue was applied onto a silica gel column with MeOH/DCM (0:100 to 1:00) to give compound 27-15 (7-((2S,3S,4R,5R)-3,4-bis(benzyloxy)-5-((benzyloxy)methyl)tetrahydrofuran-2-yl)imidazo[2,1-f][1,2,4]triazin-4-amine, 5.0 g, 76.4%) as a yellow foam. LCMS: ESI-MS: m/z=538.1 [M+H]⁺, 560.1 [M+Na]⁺.

To a solution of compound 27-15 (1.0 g, 1.86 mmol) in DCM (10.0 mL) was added BCl₃ (1 M, 11.16 mL) drop-wise at −70° C. under N₂ over 10 mins. The mixture was warmed to 0° C. and stirred for 2 h. The reaction was quenched with MeOH (50 mL) at 0° C. and concentrated under reduced pressure at 30° C. The residue was dissolved in MeOH (50 mL) and adjusted pH=10 with NH₃.H₂O (5 mL). The mixture was stirred for 1 h at 30° C. After concentrating under reduced pressure, the residue was applied onto a silica gel column with DCM/MeOH/NH₃.H₂O (10:1:1% to 5:1:1%) to give compound 27-16 ((2S,3R,4S,5R)-2-(4-aminoimidazo[2,1-f][1,2,4]triazin-7-yl)-5-(hydroxymethyl)tetrahydrofuran-3,4-diol, 600 mg, crude) as a white solid. LCMS: ESI-MS: m/z=267.9 [M+H]⁺.

To a solution of compound 27-16 (300 mg, 1.1 mmol) in pyridine (5.0 mL) was added chloro-[chloro(diisopropyl)silyl]oxy-diisopropyl-silane (424 mg, 1.34 mmol, 428 μL). The mixture was stirred at 25° C. for 12 h. The reaction was quenched with saturated NH₄Cl (30 mL) and the resulting solution was extracted with EA (50 mL). The organic layer was washed with brine (60 mL) and dried over anhydrous Na₂SO₄. After concentrating under reduced pressure, the residue was applied onto a silica gel column with PE/EA (20:1 to 3:1) to give compound 27-17 (6aR,8S,9S,9aS)-8-(4-aminoimidazo[2,1-f][1,2,4]triazin-7-yl)-2,2,4,4-tetraisopropyltetrahydro-6H-furo[3,2-f][1,3,5,2,4]trioxadisilocin-9-ol, 270 mg, 46.8%) as colorless oil. LCMS: ESI-MS: m/z=510.3 [M+H]⁺.

To a solution of compound 27-17 (270 mg, 530 μmol) in ACN (6.0 mL) was added IBX (297 mg, 1.1 mmol). The mixture was stirred at 90° C. for 3 h. The mixture was diluted with ACN (20 mL) and filtered. After concentrating under reduced pressure, the residue was applied onto a silica gel column with PE/EA (20:1 to 5:1) to give compound 27-18 ((6aR,8S,9aR)-8-(4-aminoimidazo[2,1-f][1,2,4]triazin-7-yl)-2,2,4,4-tetraisopropyldihydro-6H-furo[3,2-][1,3,5,2,4]trioxadisilocin-9(8H)-one, 148 mg, 53.4%) as light yellow oil. LCMS: ESI-MS: m/z=508.2 [M+H]⁻.

To a solution of ethynyl(trimethyl)silane (58.03 mg, 590.90 μmol) in Et₂O (3.0 mL) was added drop-wise n-BuLi (2.5 M, 189 μL) at 0° C. The mixture was stirred at 0° C. for 1 h. A mixture solution of compound 27-18 (30 mg, 59 μmol) in Et₂O (3.0 mL) was added drop-wise to the above solution at 0° C. and stirred at 0° C. for another 1 h. The reaction was quenched with saturated NH₄Cl solution (5 mL) and the resulting mixture was extracted twice with EA (10 mL). The organic phase was washed with brine (20 mL), dried over anhydrous Na₂SO₄ and concentrated under reduced pressure.

To a solution of ethynyl(trimethyl)silane (464 mg, 4.7 mmol) in Et₂O (6.0 mL) was added drop-wise n-BuLi (2.5 M, 1.51 mL) at 0° C. The mixture was stirred at 0° C. for 1 h. A mixture solution of compound 13 (6aR,8S,9aR)-8-(4-aminoimidazo[2,1-f][1,2,4]triazin-7-yl)-2,2,4,4-tetraisopropyldihydro-6H-furo[3,2-f][1,3,5,2,4]trioxadisilocin-9(8H)-one, 240 mg, 472 μmol) in Et₂O (6.0 mL) was added drop-wise to the above solution at 0° C. and stirred at 0° C. for another 1 h. The reaction was quenched with saturated NH₄Cl solution (30 mL) and the resulting solution was extracted twice with EA (30 mL). The organic phase was washed with brine (40 mL) and dried over anhydrous Na₂SO₄. After concentrating under reduced pressure, the residue was purified by prep-HPLC (water (10 mM NH₄HCO₃)—ACN) to give compound 27-19A ((6aR,8S,9S,9aR)-8-(4-aminoimidazo[2,1-f][1,2,4]triazin-7-yl)-2,2,4,4-tetraisopropyl-9-((trimethylsilyl)ethynyl)tetrahydro-6H-furo[3,2-f][1,3,5,2,4]trioxadisilocin-9-ol, 18.2 mg, 5.6%) and compound 27-19B ((6aR,8S,9R,9aR)-8-(4-aminoimidazo[2,1-f][1,2,4]triazin-7-yl)-2,2,4,4-tetraisopropyl-9-((trimethylsilyl)ethynyl)tetrahydro-6H-furo[3,2-f][1,3,5,2,4]trioxadisilocin-9-ol, 196 mg, 60.8%) as light yellow oils. 27-19A: ¹H NMR (400 MHz, CD₃OD) δ=8.14 (s, 1H), 7.67 (s, 1H), 5.58 (s, 1H), 4.70 (d, J=8.8 Hz, 1H), 4.19-4.23 (m, 1H), 3.97-4.20 (m, 2H), 3.40 (s, 1H), 1.07-1.55 (s, 28H), −0.19 (s, 9H). LCMS ESI-MS: m/z=606.2 [M+H]⁺. 27-19B: ¹H NMR (ES3943-365-P1B2): ¹H NMR (400 MHz, CD₃OD) δ=8.10 (s, 1H), 7.73 (s, 1H), 5.50 (s, 1H), 4.39 (t, J=2 Hz, 1H), 4.13 (d, J=8 Hz, 1H), 3.99-4.02 (m, 4H), 1.08-1.11 (s, 28H), −0.13 (s, 9H). LCMS: ESI-MS: m/z=606.3 [M+H]⁺.

To a solution of compound 27-19A (8 mg, 29.7 μmol) in MeOH (1.0 mL) was added NH₄F (11 mg, 297 μmol). The mixture was stirred at 60° C. for 3 h. After concentrating under reduced pressure, the residue was applied onto a silica gel column with DCM/MeOH (20:1 to 10:1) to give compound 27 (give (2S,3R,4R,5R)-2-(4-aminoimidazo[2,1-f][1,2,4]triazin-7-yl)-3-ethynyl-5-(hydroxymethyl)tetrahydrofuran-3,4-diol, 7 mg, 83%) as white solid, ¹H NMR (400 MHz, CD₃OD) δ=8.05 (s, 1H), 7.77 (s, 1H), 5.52 (s, 1H), 4.32 (d, J=7.6 Hz, 1H), 3.90-3.99 (m, 2H), 3.80 (dd, J=12.4, 4.8 Hz, 1H), 2.68 (s, 1H). ESI-MS: m/z=292.09 [M+H]+.

### Example 17

### Compound 28: (2R,3R,4R,5S)-5-(4-aminoimidazo[2,1-f][1,2,4]triazin-7-yl)-4-ethynyl-4-fluoro-2-(hydroxymethyl)tetrahydrofuran-3-ol

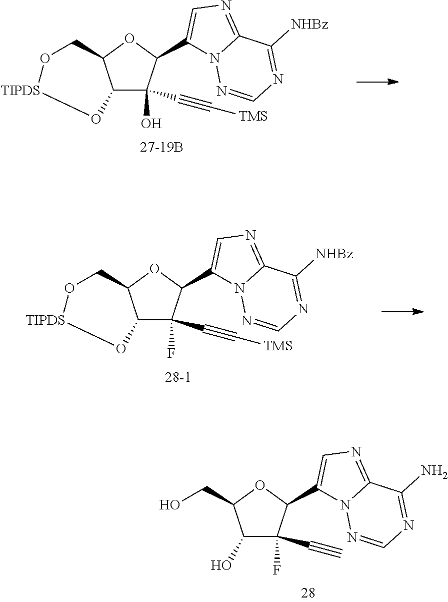

To a solution of compound 27-19B ((6aR,8S,9R,9aR)-8-(4-aminoimidazo[2,1-f][1,2,4]triazin-7-yl)-2,2,4,4-tetraisopropyl-9-((trimethylsilyl)ethynyl)tetrahydro-6H-furo[3,2-f][1,3,5,2,4]trioxadisilocin-9-ol, 215 mg, 303 μmol) in DCM (8.0 mL) was added DAST (195 mg, 1.2 mmol) dropwise at −78° C. The mixture was stirred at −78° C. for 2 h. The reaction was quenched with saturated NaHCO₃ solution (5 mL) and the aqueous phase was extracted with DCM (30 mL×2). The combined organic phase was washed with brine (15 mL) and dried over anhydrous Na₂SO₄. After concentrating under reduced pressure, the residue was applied onto a silica gel column with PE/EA (1:0 to 4:25) to give 28-1 (N-(7-((6aR,8S,9S,9aR)-9-fluoro-2,2,4,4-tetraisopropyl-9-((trimethylsilyl)ethynyl)tetrahydro-6H-furo[3,2-f][1,3,5,2,4]trioxadisilocin-8-yl)imidazo[2,1-f][1,2,4]triazin-4-yl)benzamide, 75 mg, 31.3%) as a brown solid. LCMS: ESI-MS: m/z=712.4 [M+H]⁺.

To a solution of 28-1 (87 mg, 122 μmol) in MeOH (8.0 mL) was added NH₄F (136 mg, 3.7 mmol) in one portion at 25° C. under N₂. The mixture was stirred at 90° C. for 3 h and the mixture was treated with NH₃.H₂O (1.50 mL, 28%) and stirred at 90° C. for 1.5 h. The mixture was cooled to 25° C. and concentrated under reduced pressure. The residue was applied onto a silica gel column with DCM/MeOH (30:1 to 10:1) to give compound 28 ((2R,3R,4R,5S)-5-(4-aminoimidazo[2,1-f][1,2,4]triazin-7-yl)-4-ethynyl-4-fluoro-2-(hydroxymethyl)tetrahydrofuran-3-ol, 28 mg, 75%) as a white solid. ESI-MS: m/z=294.09 [M+H]⁻. ¹H NMR (400 MHz, CD₃OD) δ=8.06 (s, 1H), 7.75 (s, 1H), 5.74 (d, J=22.8 Hz, 1H), 4.49 (dd, J=9.2, 19.6 Hz, 1H), 3.94-3.97 (m, 2H), 3.76-3.81 (m, 1H), 3.00 (d, J=5.2 Hz, 1H). ¹⁹F NMR (376 Hz, CD₃OD) δ=−154.639.

### Example 18

### Compound 29: (2R,3R,4R,5R)-2-(6-amino-2-chloro-9H-purin-9-yl)-3-ethynyl-5-(hydroxymethyl)tetrahydrofuran-3,4-diol

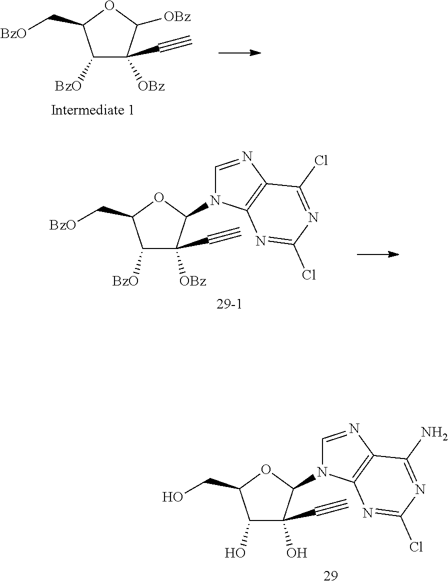

To a solution of Intermediate 1 ((2R,3R,4R,5R)-5-((benzoyloxy)methyl)-3-ethynyltetrahydrofuran-2,3,4-triyltribenzoate, 500 mg, 846.6 μmol) in ACN (5.0 mL) was added DBU (773 mg, 5.1 mmol) at 0° C. and stirred for 15 min. TMSOTf (1.51 g, 6.8 mmol, 1.2 mL) was added at 0° C. and the mixture was stirred for 15 mins and then at 70° C. for 12 h. The reaction was cooled to room temperature and diluted with EA (10 mL). The resulting solution was washed with sat. NaHCO₃ solution (50 mL×3) and brine (50 mL×3). The organic layer was dried over anhydrous Na₂SO₄. After concentrating under reduced pressure, the residue was applied onto a silica gel column with PE/EA (1:10 to 0:10) to give compound 29-1 ((2R,3R,4R,5R)-5-((benzoyloxy)methyl)-2-(2,6-dichloro-9H-purin-9-yl)-3-ethynyltetrahydrofuran-3,4-diyldibenzoate, 220 mg, 39.5%) as a white solid. LCMS: ESI-MS: m/z=658.8 [M+H]⁺.

To a solution of compound 29-1 (100 mg, 152 μmol) in THF (2.0 mL) was added NH₃ (7 M, in MeOH, 5.0 mL). The mixture was stirred at 50° C. for 24 h. After concentrating under reduced pressure, the residue was applied onto a silica gel column with MeOH/DCM (0:1 to 1:10) to give compound 29 ((2R,3R,4R,5R)-2-(6-amino-2-chloro-9H-purin-9-yl)-3-ethynyl-5-(hydroxymethyl)tetrahydrofuran-3,4-diol, 24 mg, 44%) as a white solid. ¹H NMR (400 MHz, DMSO-d₆) δ=8.44 (s, 1H), 7.83 (s, 2H), 6.46 (s, 1H), 5.95 (s, 1H), 5.74 (d, J=7.5 Hz, 1H), 5.21 (t, J=5.0 Hz, 1H), 4.40 (t, J=8.3 Hz, 1H), 3.90 (d, J=8.5 Hz, 1H), 3.83-3.74 (m, 1H), 3.74-3.54 (m, 1H), 3.21 (s, 1H). LCMS: ESI-MS: m/z=326.2 [M+H]⁺.

### Example 19

### Compound 30: 9-((2R,3R,4S,5S)-3-ethynyl-5-fluoro-3,4-dihydroxy-5-(hydroxymethyl)tetrahydrofuran-2-yl)-1,9-dihydro-6H-purin-6-one

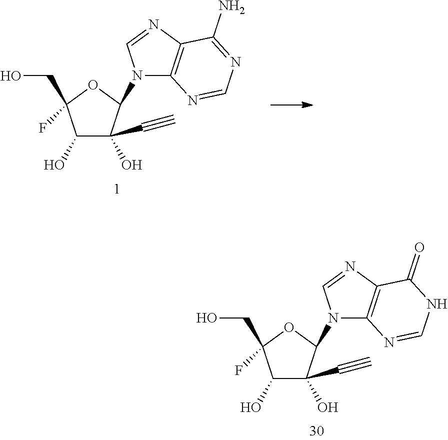 [SMILES: C#C[C@]1(O)[C@H](n2cnc3c(N)ncnc32)O[C@](F)(CO)[C@H]1O ; C#C[C@]1(O)[C@H](n2cnc3c(=O)[nH]cnc32)O[C@](F)(CO)[C@H]1O]

To a mixture of compound 1 (31 mg, 0.1 mmol) in glacial acetic acid (0.5 mL) was added 4 M aq. solution of NaNO₂ (50 μL, 0.2 mmol). Addition of the same amount of NaNO₂ solution was repeated 3 times in 8 h or 12 h intervals. Mixture was then concentrated and purified by RP-HPLC (0-30% B, A: 50 mM aq. TEAA, B: 50 mM TEAA in ACN) to provide compound 30 (25 mg, 81%). ¹H-NMR (DMSO-d₆): δ 8.20, 8.07 (2 s, 2H, H-2, H-8), 6.28 (s, 1H, H-1′), 6.5, 6.1, 5.7 (3 br, 3×1H, 3OH), 4.59 (d, J=19.6 Hz, 1H, H-3′), 3.62. (m, 2H, H-5′a, H-5′b), 3.22 (s, 1H, C≡CH). ¹⁹F-NMR (DMSO-d₆): δ −120.59 (m). MS m/z=309.0 (M−1).

### Example 20

### Compound 31: (2S,3S,4R,5R)-5-(4-amino-5-fluoro-7H-pyrrolo[2,3-d]pyrimidin-7-yl)-4-ethynyl-2-fluoro-2-(hydroxymethyl)tetrahydrofuran-3,4-diol

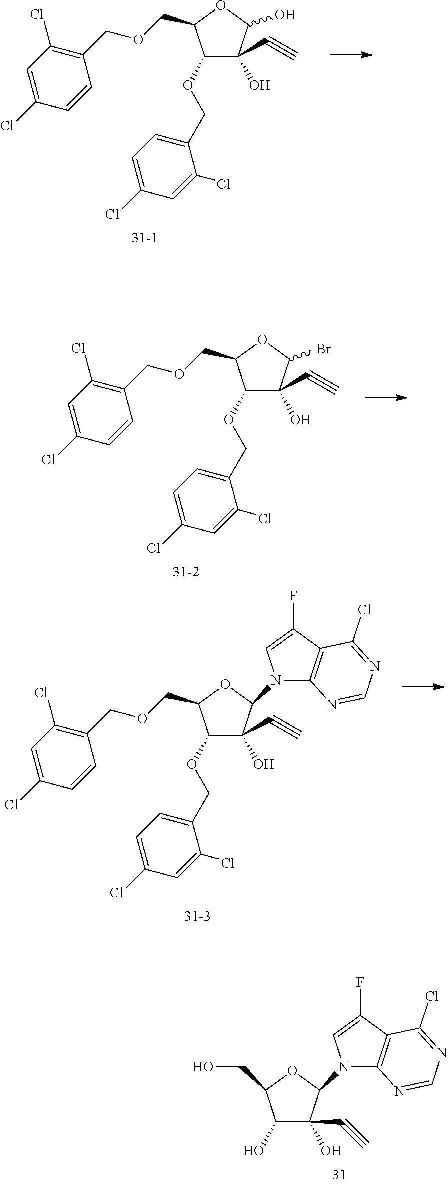 [SMILES: C#C[C@]1(O)C(O)O[C@H](COCc2ccc(Cl)cc2Cl)[C@H]1OCc1ccc(Cl)cc1Cl ; C#C[C@]1(O)C(Br)O[C@H](COCc2ccc(Cl)cc2Cl)[C@H]1OCc1ccc(Cl)cc1Cl ; C#C[C@@]1(O)[C@H](OCc2ccc(Cl)cc2Cl)[C@@H](COCc2ccc(Cl)cc2Cl)O[C@H]1n1cc(F)c2c(Cl)ncnc21 ; C#C[C@@]1(O)[C@H](O)[C@@H](CO)O[C@H]1n1cc(F)c2c(Cl)ncnc21]

Compound 31-1 ((3R,4R,5R)-4-((2,4-dichlorobenzyl)oxy)-5-(((2,4-dichlorobenzyl)oxy)methyl)-3-ethynyltetrahydrofuran-2,3-diol, 700 mg, 1.43 mmol) was dissolved in DCM (15 mL) and 33% HBr in AcOH (0.42 mL, 7.14 mmol, 5 eq.) was added to this at R.T. After stirring for 1 h 45 min, the solvent was evaporated to dryness and co-evaporated with anhydrous toluene (2×25 mL) to provide compound 31-2 ((3R,4R,5R)-2-bromo-4-((2,4-dichlorobenzyl)oxy)-5-(((2,4-dichlorobenzyl)oxy)methyl)-3-ethynyltetrahydrofuran-3-ol), which was used directly in the next step without further purification.

7-fluoro-6-chloroadenine (366 mg, 2.13 mmol, 1.5 eq.) was suspended in ACN (15 ml) and NaH (103 mg, 4.26 mmol, 3.0 eq.) was added at R.T. After stirring for 30 min at R.T., to compound 31-2 in ACN (20 mL) was added under argon. The mixture was stirred at R.T. overnight and quenched with citric acid solution (20 mL). EA (30 mL) was added and washed with sat. aq. NaHCO₃ (1×15 mL) and sat. aq. NaCl (1×15 mL). The organic phase was evaporated to dryness and the resulting crude was purified by silica gel column chromatography (10-50% EA in Hexane, v/v) to afford compound 31-3 ((2R,3R,4R,5R)-2-(4-chloro-5-fluoro-7H-pyrrolo[2,3-d]pyrimidin-7-yl)-4-((2,4-dichlorobenzyl)oxy)-5-(((2,4-dichlorobenzyl)oxy)methyl)-3-ethynyltetrahydrofuran-3-ol, 450 mg, 45%) as a white solid. MS m/z (ESI): [645.95 M+H]⁺.

Compound 31-3 (350 mg, 0.545 mmol) was coevaporated with an toluene (2×10 mL) and dissolved in anhydrous DCM (15 mL) and cooled to −78° C. BCl₃ in DCM (5.5 mL, 5.5 mmol, 1 M) was added to and the mixture was stirred for 3 h at −78° C. The mixture was allowed to warm to 0° C. and MeOH (15 mL) was added and stirred for 30 min. The reaction was neutralized with aq. NH₃ (1.3 mL) and filtered. The filtrate was evaporated to dryness and purified by purified by silica gel column chromatography (0-20% MeOH in DCM, v/v) to afford compound 31 ((2S,3S,4R,5R)-5-(4-amino-5-fluoro-7H-pyrrolo[2,3-d]pyrimidin-7-yl)-4-ethynyl-2-fluoro-2-(hydroxymethyl)tetrahydrofuran-3,4-diol, 91 mg, 52%) as a white solid. MS m/z (ESI): 309.00 [M+H]⁺, ¹H-NMR (400 MHz, CD3OD-d₃): δ ppm 8.05 (s, 1H, H2/H8), 7.34 (S, 1H, 1H), 6.31 (d, J=1.6 Hz, 1H), 4.41 (d, J=9.2 Hz, 1H), 3.92-3.98 (m, 2H), 3.75-3.95 (m, 1H), 2.56 9 s, 1H), ¹⁹F-NMR (376.40 MHz, DMSO-d₆): δ ppm −167.85 (multiplet).

### Example 21

### Compound 34: (2S,3S,4R,5R)-2-(acetoxymethyl)-5-(6-amino-9H-purin-9-yl)-4-ethynyl-2-fluorotetrahydrofuran-3,4-diyldiacetate

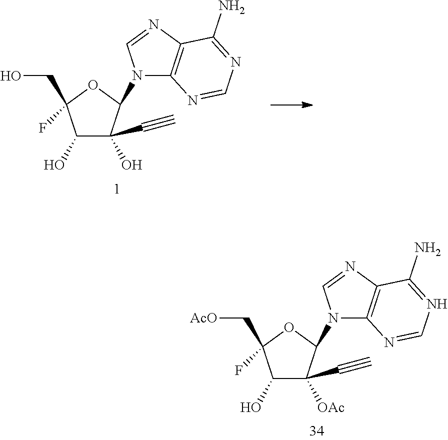 [SMILES: C#C[C@]1(O)[C@H](n2cnc3c(N)ncnc32)O[C@](F)(CO)[C@H]1O ; C#C[C@]1(OC(C)=O)[C@H](n2cnc3c(N)ncnc32)O[C@](F)(COC(C)=O)[C@H]1O]

To an ice-cold mixture of compound 1 (2S,3S,4R,5R)-5-(6-amino-9H-purin-9-yl)-4-ethynyl-2-fluoro-2-(hydroxymethyl)tetrahydrofuran-3,4-diol, 50 mg, 0.16 mmol), acetic anhydride (61 μL, 0.64 mmol) and Et₃N (0.11 mL, 0.8 mmol) in ACN (2 mL) was added DMAP (4 mg, 0.03 mmol) and the resulting solution stirred at 0° C. for 1 h. Reaction was quenched with MeOH and the mixture evaporated. Purification on silica gel column with iPrOH/DCM (4:100 to 15:100) provided 45 mg (65%) of 34 ((2S,3S,4R,5R)-2-(acetoxymethyl)-5-(6-amino-9H-purin-9-yl)-4-ethynyl-2-fluorotetrahydrofuran-3,4-diyldiacetate). ¹H-NMR (CDCl₃): δ 8.38, 8.01 (2 s, 2H, H-2, H-8), 6.69 (s, 1H, H-1′), 6.51 (d, J=14.0 Hz, 1H, H-3′), 5.69 (br s, 2H, NH₂), 4.55 (m, 2H, H-5′a, H-5′b), 2.46 (s, 1H, C≡CH), 2.12, 2.19, 2.21 (3 s, 3×3H, 3 Me). MS m/z=435.90 [M+1]⁺.

### Example 22

### Compound 36: 2S,3S,4R,5R)-5-(6-amino-9H-purin-9-yl)-4-ethynyl-2-fluoro-4-hydroxy-2-((propionyloxy)methyl)tetrahydrofuran-3-ylpropionate

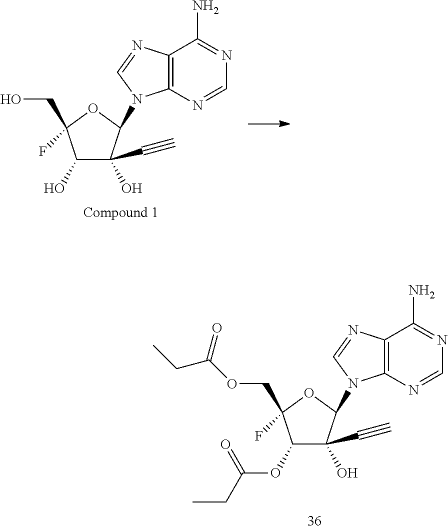 [SMILES: C#C[C@]1(O)[C@H](n2cnc3c(N)ncnc32)O[C@](F)(CO)[C@H]1O ; C#C[C@]1(O)[C@H](n2cnc3c(N)ncnc32)O[C@](F)(COC(=O)CC)[C@H]1OC(=O)CC]

Compound 1 (50 mg, 0.161 mmol) was co-evaporated with anhydrous toluene (2×10 mL) and dissolved in anhydrous ACN (1 mL). Pyridine (65 μL, 0.809 mmol) and propionic anhydride (52 μL, 0.404 mmol) were added at R.T. After stirring the mixture at R.T. overnight, EA (30 mL) was added and washed with sat. aq. NaHCO₃ (1×15 mL) and sat. aq. NaCl (1×15 mL). After evaporating the solvent under reduced pressure, the residue was purified by prep-HPLC (Buffer A: 0.1% formic acid in H₂O and Buffer B: 0.1% formic acid in ACN, gradient 25-85% of Buffer B in 20 min) to afford compound 36 (2S,3S,4R,5R)-5-(6-amino-9H-purin-9-yl)-4-ethynyl-2-fluoro-4-hydroxy-2-((propionyloxy)methyl)tetrahydrofuran-3-ylpropionate, 34 mg, 49.2%). MS m/z (ESI): 478.05 [M+H]⁺. ¹H-NMR (400 MHz, CD₃CN-d3): δ ppm 8.25 (s, 1H, H2/H8), 8.05 (s, 1H, H2/H8), 6.51 (d, J=17.6 Hz, 1H) 6.40 (s, 1H), 6.09 (br, S, 2H, NH₂), 4.52-4.62 (m, 1H, H5′), 4.38-4.48 (m, 1H, H5′), 2.50-2.59 (m, 4H, 2×CH2), 2.30-2.40 (m, 3H, 1×CH2, 1 acetylene proton), 1.17 (t, J=8 Hz, 3H), 1.08 (t, J=8 Hz, 3H). ¹⁹F-NMR (376.40 MHz, CD₃CN-d3): δ −116.7 (multiplet).

### Example 23

### Compound 37: (2S,3S,4R,5R)-5-(6-amino-9H-purin-9-yl)-2-((butyryloxy)methyl)-4-ethynyl-2-fluoro-4-hydroxytetrahydrofuran-3-ylbutyrate

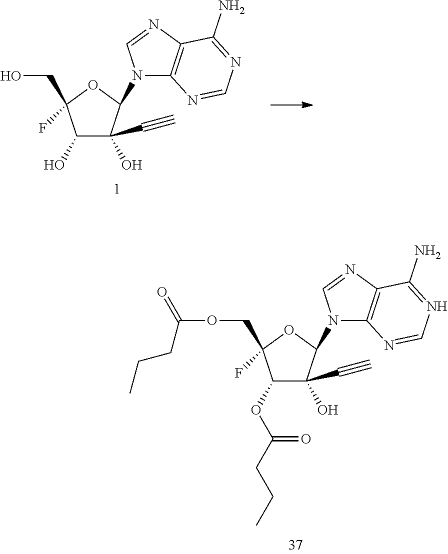 [SMILES: C#C[C@]1(O)[C@H](n2cnc3c(N)ncnc32)O[C@](F)(CO)[C@H]1O ; C#C[C@]1(O)[C@H](n2cnc3c(N)ncnc32)O[C@](F)(COC(=O)CCC)[C@H]1OC(=O)CCC]

A mixture of compound 1 (50 mg, 0.16 mmol) in pyridine (2 mL) and butyric anhydride (78 μL, 0.48 mmol) was stirred overnight at r.t. Reaction was quenched with MeOH and the mixture evaporated and coevaporated with toluene. Purification on silica gel column with iPrOH/DCM (4:100 to 15:100) provided 62 mg (86%) of 37 ((2S,3S,4R,5R)-5-(6-amino-9H-purin-9-yl)-2-((butyryloxy)methyl)-4-ethynyl-2-fluoro-4-hydroxytetrahydrofuran-3-ylbutyrate). ¹H-NMR (CDCl₃): δ 8.34, 7.97 (2 s, 2H, H-2, H-8), 6.43 (s, 1H, H-1′), 6.15 (d, J=13.2 Hz, 1H, H-3′), 5.79 (br s, 2H, NH₂), 4.50 (m, 2H, H-5′a, H-5′b), 2.45, 2.36 (2 m, 2×2H, 2×C(O)CH₂), 2.30 (s, 1H, C≡CH), 1.62-1.77 (m, 4H, 2×CH₂CH₂CH₃), 0.98 (t, J=7.2 Hz, 3H, CH₃), 0.95 (t, J=7.2 Hz, 3H, CH₃). MS m/z=450.0 [M+1]⁺.

### Example 24

### Compound 38: (2S,3S,4R,5R)-5-(6-amino-9H-purin-9-yl)-4-ethynyl-2-fluoro-2-((propionyloxy)methyl)tetrahydrofuran-3,4-diyldipropionate

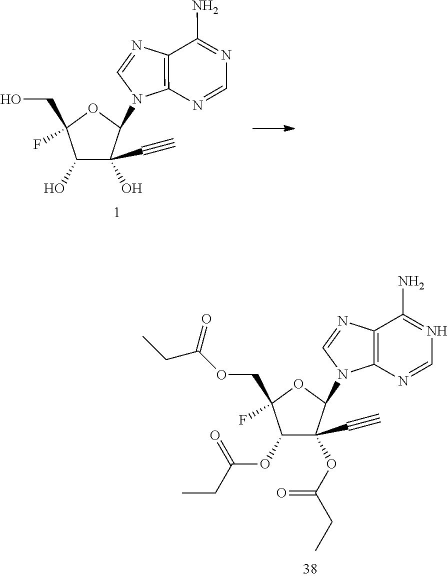 [SMILES: C#C[C@]1(O)[C@H](n2cnc3c(N)ncnc32)O[C@](F)(CO)[C@H]1O ; C#C[C@]1(OC(=O)CC)[C@H](n2cnc3c(N)ncnc32)O[C@](F)(COC(=O)CC)[C@H]1OC(=O)CC]

Compound 1 (50 mg, 0.161 mmol) was coevaporated with anhydrous toluene (2×10 mL) and dissolved anhydrous ACN (1 mL). TEA (113 μL, 0.805 mmol), DMAP (2 mg, 0.016 mmol) and propionic anhydride (88 μL, 0.680 mmol) were added at 0° C. After stirring for 90 min at 0° C., the mixture was diluted with EA (30 mL) and washed with sat. aq. NaHCO₃ (1×15 mL) and sat. aq. NaCl (1×15 mL). The resulting crude material was purified by prep-HPLC (Buffer A: 0.1% formic acid in H₂O and Buffer B: 0.1% formic acid in ACN, gradient 25-85% of Buffer B in 20 min) to afford compound 38 ((2S,3S,4R,5R)-5-(6-amino-9H-purin-9-yl)-4-ethynyl-2-fluoro-2-((propionyloxy)methyl)tetrahydrofuran-3,4-diyldipropionate, 48 mg, 62.3%). MS m/z (ESI): 478.05 [M+H]⁺. ¹H-NMR (400 MHz, CD₃CN-d3): δ ppm 8.25 (s, 1H, H2/H8), 8.02 (s, 1H, H2/H8), 6.70-6.78 (m, 1.5H, H3′/H1′), 6.68-6.73 (m, 0.5H, H3′/H1′), 6.09 (br. S, 2H, NH2), 4.56-4.67 (m, 1H, H5′), 4.44-4.55 (m, 1H, H5′), 2.63 (s, 1H, acetylene proton), 2.40-2.52 (m, 4H, 2×CH2), 2.16-2.40 (m, 2H, 1×CH2), 1.05-1.20 (m, 9H). 19F-NMR (376.40 MHz, CD₃CN-d3): δ ppm −117.7 (multiplet).

### Example 25

### Compound 39: (2S,3S,4R,5R)-5-(6-amino-9H-purin-9-yl)-4-ethynyl-2-fluoro-4-hydroxy-2-(hydroxymethyl)tetrahydrofuran-3-yldecanoate

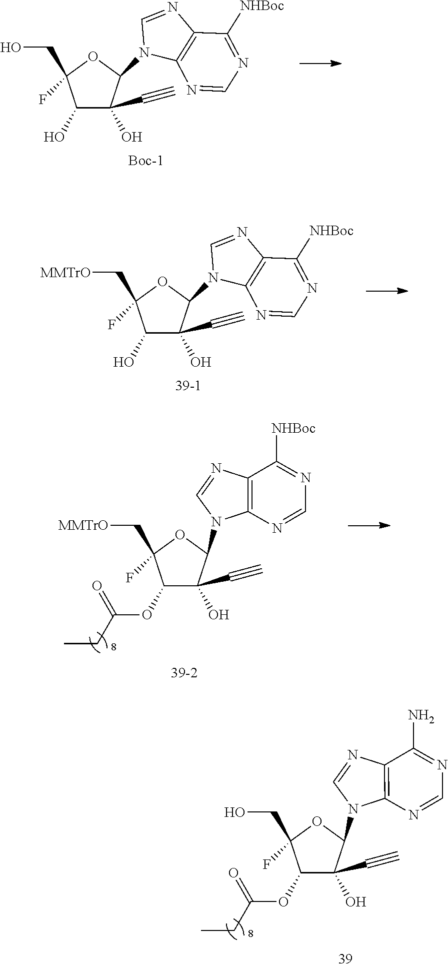

Boc-protected 1 (tert-butyl(9-((2R,3R,4S,5S)-3-ethynyl-5-fluoro-3,4-dihydroxy-5-(hydroxymethyl)tetrahydrofuran-2-yl)-9H-purin-6-yl)carbamate, 630 mg, 1.54 mmol) was co-evaporated with anhydrous pyridine (2*20 mL) and dissolved anhydrous pyridine (10 mL). Methoxytritylchloride (MMTr-Cl, 0.72 gr, 2.31 mmol) was added to this in two portions over 20 min at 0° C. After stirring the reaction mixture for overnight at R.T., it was diluted EA (60 mL) and washed with sat. aq. NaHCO₃ (1*25 mL) and sat. aq. NaCl (1×25 mL). The organic phase was evaporated to dryness and resulting crude was purified by column chromatography (0-15% MeOH in DCM:Hexane:Acetone, 5:3:2, v/v/v) to afford compound 39-1 (tert-butyl(9-((2R,3R,4S,5S)-3-ethynyl-5-fluoro-3,4-dihydroxy-5-(((4-methoxyphenyl)diphenylmethoxy)methyl)tetrahydrofuran-2-yl)-9H-purin-6-yl)carbamate, 740 mg, 71%) as a white solid. MS m/z (ESI): 682.10 [M+H]⁺.

Compound 39-1 (120 mg, 0.176 mmol) was co-evaporated with anhydrous toluene (2*10 mL) and dissolved anhydrous ACN (2 mL). Pyridine (70 μL, 0.85 mmol) and decanoicanhydride (75 mg, 0.23 mmol) were added to this at R.T. After stirring the reaction mixture at R.T. for overnight, it was diluted EA (30 mL) and washed with sat. aq. NaHCO₃ (1×15 mL) and sat. aq. NaCl (1*15 mL). The organic phase was evaporated to dryness and the resulting crude was purified by silica gel chromatography (0-70% EA in Hexane, v/v) to afford compound 39-2 ((2S,3S,4R,5R)-5-(6-((tert-butoxycarbonyl)amino)-9H-purin-9-yl)-4-ethynyl-2-fluoro-4-hydroxy-2-(((4-methoxyphenyl)diphenylmethoxy)methyl)tetrahydrofuran-3-yldecanoate, 118 mg, 80.2%) as a white solid. MS m/z (ESI): 836.30 [M+H]⁺. 3′-Decanoate nucleoside 3 (116 mg, 0.138 mmol) was subjected to HCl in ACN (0.97 mmol, 0.4M, 2.43 mL). Triethylsilane (110 μL, 0.69 mmol) was added to this and after stirring the reaction at R.T. for 16 h, it was evaporated to dryness and purified by prep-HPLC (Buffer A: 0.1% formic acid in H₂O and Buffer B: 0.1% formic acid in ACN, gradient 25-85% of Buffer B in 20 min) to afford compound 39 (((2S,3S,4R,5R)-5-(6-amino-9H-purin-9-yl)-4-ethynyl-2-fluoro-4-hydroxy-2-(hydroxymethyl)tetrahydrofuran-3-yldecanoate, 34 mg, 53.1%). MS m/z [M+H]⁺ (ESI): 464.10. ¹H-NMR (400 MHz, DMSO-d₆): δ ppm 8.29 (s, 1H, H2/H8), 8.13 (s, 1H, H2/H8), 7.33 (br. S, 2H, NH2), 6.87 (s, 1H, 2′OH), 6.39 (s, 1H), 6.02 (d, J=18 Hz, 1H), 5.63 (m, 1H, 5′OH), 3.58-3.70 (m, 1H), 2.42 (s, 1H), 1.48-1.57 (m, 2H), 1.20-1.32 (m, 14H), 0.78-0.85 (m, 3H). ¹⁹F-NMR (376.40 MHz, CD3CN-d₃): δ −119.3 (multiplet).

### Example 26

### Compound 40: 2S,3S,4R,5R)-5-(6-((tert-butoxycarbonyl)amino)-9H-purin-9-yl)-4-ethynyl-2-fluoro-4-hydroxy-2-(((4-methoxyphenyl)diphenylmethoxy)methyl)tetrahydrofuran-3-yl)-octanoate

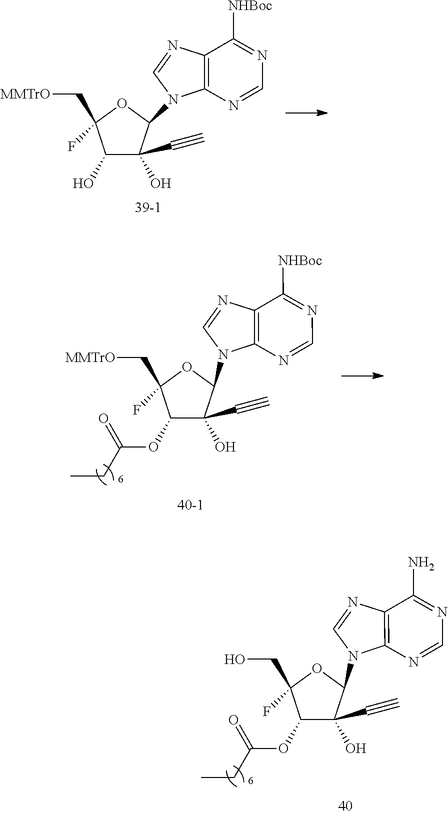

Octonoic acid (47 mg, 0.323 mmol) and CDI (53 mg, 0.323 mmol) was dissolved in ACN (2 mL). This mixture was stirred for 1 h at R.T. to generate the activated acid. Compound 39-1 (147 mg, 0.215 mmol) was co-evaporated with anhydrous toluene (2*10 mL) and dissolved anhydrous ACN (1 mL), and trimethylamine (60 μL, 0.430 mmol) was added and the mixture cooled to 0° C. The activated acid was added over 2 min at 0° C. After stirring for 6 h, the reaction was diluted with EA (30 mL) and washed with sat. aq. NaHCO₃ (1*15 mL) and sat. aq. NaCl (1*15 mL). The organic phase was evaporated to dryness and the crude material was purified by silica gel chromatography 0-70% EA in Hexane, v/v) to afford compound 40-1 ((2S,3S,4R,5R)-5-(6-((tert-butoxycarbonyl)amino)-9H-purin-9-yl)-4-ethynyl-2-fluoro-4-hydroxy-2-(((4-methoxyphenyl)diphenylmethoxy)methyl)tetrahydrofuran-3-yloctanoate, 127 mg, 72.9%) as a white solid. MS m/z [M+H]⁺ (ESI): 808.20.

Compound 40-1 (125 mg, 0.154 mmol) was treated with HCl in ACN (1.08 mmol, 2.8 mL). Triethylsilane (197 μL, 1.23 mmol) was added, and after stirring at R.T. for 48 h, the volatiles were removed under reduced pressure and the residue was purified by prep-HPLC (Buffer A: 0.1% formic acid in H₂O and Buffer B: 0.1% formic acid in ACN, gradient 25-85% of Buffer B in 20 min) to afford 40 (2S,3S,4R,5R)-5-(6-((tert-butoxycarbonyl)amino)-9H-purin-9-yl)-4-ethynyl-2-fluoro-4-hydroxy-2-(((4-methoxyphenyl)diphenylmethoxy)methyl)tetrahydrofuran-3-yl-octanoate, 32 mg, 47.2%). MS m/z [M+H]⁻ (ESI): 436.00. ¹H-NMR (400 MHz, DMSO-d₆): δ ppm 8.29 (s, 1H, H2/H8), 8.13 (s, 1H, H2/H8), 7.33 (br. S, 2H, NH2), 6.87 (s, 1H, 2′OH), 6.39 (s, 1H), 6.02 (d, J=18 Hz, 1H), 5.63 (m, 1H, 5′OH), 3.58-3.72 (m, 2H), 2.40-2.52 (m, 3H), 1.50-1.58 (m, 2H), 1.20-1.32 (m, 8H), 0.80-0.86 (m, 3H). ¹⁹F-NMR (376.40 MHz, DMSO-d₆): δ −117.6 (multiplet).

### Example 27

### Compound 41: (2S,3S,4R,5R)-5-(6-amino-9H-purin-9-yl)-4-ethynyl-2-fluoro-4-hydroxy-2-(hydroxymethyl)tetrahydrofuran-3-yl L-valinate

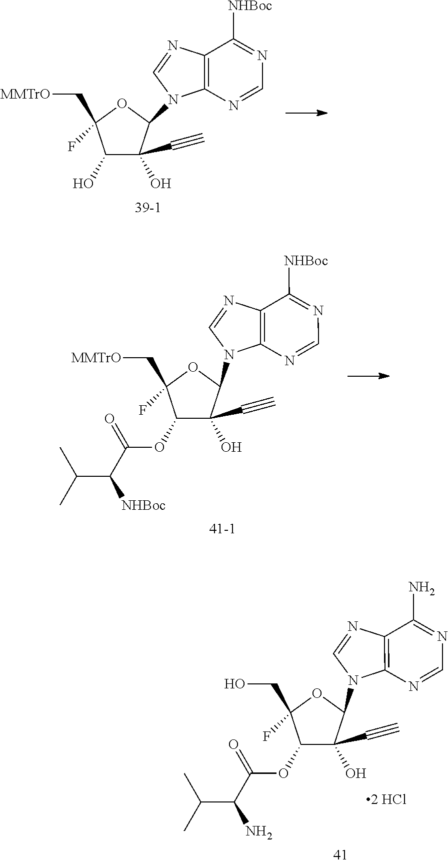

Boc-Val-OH (73 mg, 0.332 mmol) and CDI (55 mg, 0.332 mmol) was dissolved in ACN (1 mL). This mixture was stirred for 1 h at R.T. to generate the activated amino acid. Compound 39-1 (150 mg, 0.221 mmol) was co-evaporated with anhydrous toluene (2*10 mL) and dissolved anhydrous ACN (1 mL), and trimethylamine (63 μL, 0.440 mmol) was added and the mixture cooled to 0° C. The activated amino-acid was added over 2 min at 0° C. After stirring the mixture at R.T. for 2 h, EA (30 mL) was added and washed with sat. aq. NaHCO₃ (1*15 mL) and sat. aq. NaCl (1*15 mL). The organic phase was evaporated to dryness and the crude material was purified by prep-HPLC (Buffer A: 0.1% formic acid in H₂O and Buffer B: 0.1% formic acid in ACN, gradient 60-95% of Buffer B in 20 min) to afford compound 41-1 ((2S,3S,4R,5R)-5-(6-((tert-butoxycarbonyl)amino)-9H-purin-9-yl)-4-ethynyl-2-fluoro-4-hydroxy-2-(((4-methoxyphenyl)diphenylmethoxy)methyl)tetrahydrofuran-3-yl(tert-butoxycarbonyl)-L-valinate, 92 mg, 47.4%). MS m/z [M+H]⁺ (ESI): 881.20

Compound 41-1 (92 mg, 0.104 mmol) was treated with HCl in ACN (1.04 mmol, 0.4M, 2.6 mL). Triethylsilane (133 μL, 0.832 mmol) was added and after stirring the mixture at R.T. for 48 h, the reaction was further diluted with Et₂O (30 mL) and resulting precipitate filtered and washed with excess Et₂O to afford compound 41 as a di-hydrochloride salt ((2S,3S,4R,5R)-5-(6-amino-9H-purin-9-yl)-4-ethynyl-2-fluoro-4-hydroxy-2-(hydroxymethyl)tetrahydrofuran-3-yl L-valinate, 32 mg, 76.1%). MS m/z [M+H]⁺ (ESI): 408.95. ¹H-NMR (400 MHz, DMSO-d₆): δ ppm 8.58-8.65 (m, 2H), 8.57 (s, 1H, H2/H8), 8.44 (s, 1H, H2/H8), 7.06 (s, 1H), 6.50 (s, 1H), 6.12 (d, J=17.2 Hz, 1H), 4.10-4.15 (m, 1H), 3.60-3.82 (m, 5H) 3.34 (s, H), 2.20-2.36 (m, 1H) 0.92-1.03 (m, 6H). ¹⁹F-NMR (376.40 MHz, DMSO-d₆): δ−117.1 (multiplet).

### Example 28

### Compound 42: (2S,3S,4R,5R)-5-(6-((tert-butoxycarbonyl)amino)-9H-purin-9-yl)-4-ethynyl-2-fluoro-4-hydroxy-2-(((4-methoxyphenyl)diphenylmethoxy)methyl)tetrahydrofuran-3-yl-dodecanoate

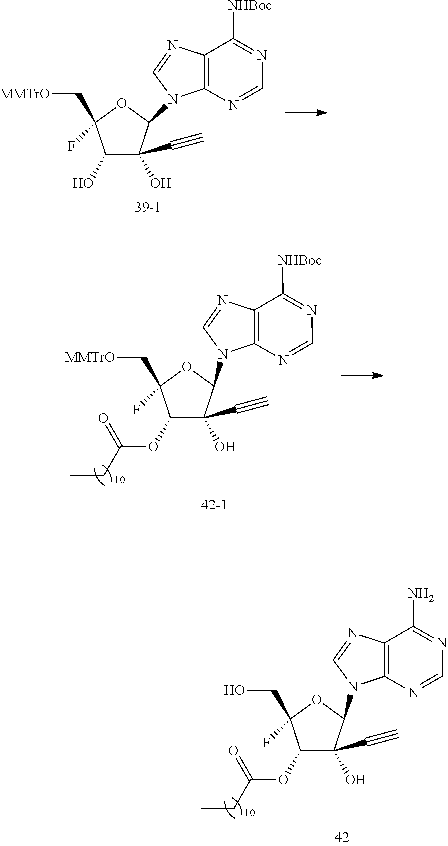

Compound 39-1 (130 mg, 0.190 mmol) was co-evaporated with anhydrous toluene (2×10 mL) and dissolved anhydrous ACN/DCM (2:1, 3 mL). Pyridine (77 μL, 0.95 mmol) and dodecanoic anhydride (102 mg, 0.27 mmol) were added at R.T. After stirring the reaction mixture at R.T. overnight, the mixture was diluted with EA (30 mL) and washed with sat. aq. NaHCO₃ (1*15 mL) and sat. aq. NaCl (1*15 mL). The organic phase was evaporated to dryness and the crude material was purified by silica gel chromatography (0-70% EA in Hexane, v/v) to afford compound 42-1 (((2S,3S,4R,5R)-5-(6-((tert-butoxycarbonyl)amino)-9H-purin-9-yl)-4-ethynyl-2-fluoro-4-hydroxy-2-(((4-methoxyphenyl)diphenylmethoxy)methyl)tetrahydrofuran-3-yldodecanoate 140 mg, 84.8%) as a white solid. MS m/z [M+H]⁺ (ESI): 864.30.

Compound 42-1 (140 mg, 0.162 mmol) was treated with HCl in ACN (1.29 mmol, 0.4M, 3.3 mL). Triethylsilane (206 μL, 1.29 mmol) was added, and after stirring at R.T. for 16 h, the volatiles were removed under reduced pressure and the residue was purified by prep-HPLC (Buffer A: 0.1% formic acid in H₂O and Buffer B: 0.1% formic acid in ACN, gradient 35-85% of Buffer B in 20 min) to afford compound 42 ((2S,3S,4R,5R)-5-(6-((tert-butoxycarbonyl)amino)-9H-purin-9-yl)-4-ethynyl-2-fluoro-4-hydroxy-2-(((4-methoxyphenyl)diphenylmethoxy)methyl)tetrahydrofuran-3-yl-dodecanoate 37 mg, 46.2%). MS m/z [M+H]⁺ (ESI): 492.10. ¹H-NMR (400 MHz, DMSO-d₆): δ ppm 8.29 (s, 1H, H2/H8), 8.13 (s, 1H, H2/H8), 7.33 (br. S, 2H, NH2), 6.87 (s, 1H, 2′—OH), 6.39 (s, 1H), 6.02 (d, J=17.6 Hz, 1H), 5.63 (m, 1H, 5′—OH), 3.58-3.70 (m, 1H), 2.40-2.45 (m, 3H), 1.48-1.57 (m, 2H), 1.15-1.35 (m, 18H), 0.82 (t, J=6.8 Hz, 3H). ¹⁹F-NMR (376.40 MHz, DMSO-d₆): δ−117.7 (multiplet).

### Example 29

### Compound 43: (2S,3S,4R,5R)-5-(6-amino-9H-purin-9-yl)-4-ethynyl-2-fluoro-2-((isobutyryloxy)methyl)tetrahydrofuran-3,4-diylbis(2-methylpropanoate)

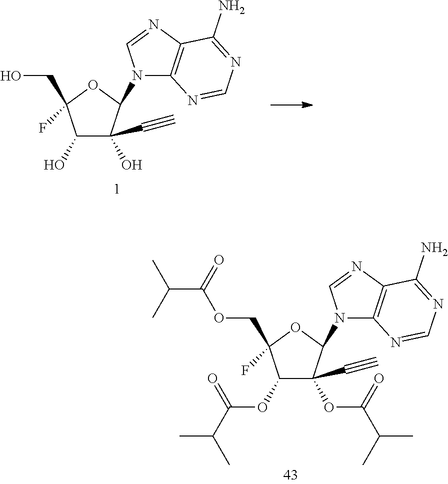 [SMILES: C#C[C@]1(O)[C@H](n2cnc3c(N)ncnc32)O[C@](F)(CO)[C@H]1O ; C#C[C@]1(OC(=O)C(C)C)[C@H](n2cnc3c(N)ncnc32)O[C@](F)(COC(=O)C(C)C)[C@H]1OC(=O)C(C)C]

To an ice-cold mixture of compound 1 ((2S,3S,4R,5R)-5-(6-amino-9H-purin-9-yl)-4-ethynyl-2-fluoro-2-(hydroxymethyl)tetrahydrofuran-3,4-diol, 50 mg, 0.16 mmol), isobutyric anhydride (0.11 mL, 0.64 mmol) and Et₃N (0.11 mL, 0.8 mmol) in ACN (2 mL) was added DMAP (4 mg,0.03 mmol) and the resulting solution stirred at 0° C. for 1 h. Reaction was quenched with MeOH and the mixture evaporated. Purification on silica gel column with iPrOH/DCM (3:100 to 10:100) provided 70 mg (85%) of 43 ((2S,3S,4R,5R)-5-(6-amino-9H-purin-9-yl)-4-ethynyl-2-fluoro-2-((isobutyryloxy)methyl)tetrahydrofuran-3,4-diylbis(2-methylpropanoate)). ¹H-NMR (DMSO-d₆): δ 8.18, 8.14 (2 s, 2H, H-2, H-8), 7.38 (br s, 2H, NH₂), 6.38 (s, 1H, H-1′), 6.74 (d, J=18.0 Hz, 1H, H-3′), 4.49 (m, 2H, H-5′a, H-5′b), 3.52 (s, 1H, C≡CH), 2.61-2.73 (m, 2H, 2*CHMe₂), 2.51 (m, 1H, CHMe₂), 1.12-1.16 (m, 12H, 2*CHMe₂), 1.06, 1.04 (2 d, J=7.0 Hz, 2*3H, CHMe₂). ¹⁹F-NMR (DMSO-d₆): δ −116.58 (m). MS m/z=520.05 [M+1]⁺.

### Example 30

### Compound 44: ((2S,3S,4R,5R)-5-(6-amino-9H-purin-9-yl)-4-ethynyl-2-fluoro-3,4-dihydroxytetrahydrofuran-2-yl)methyldecanoate

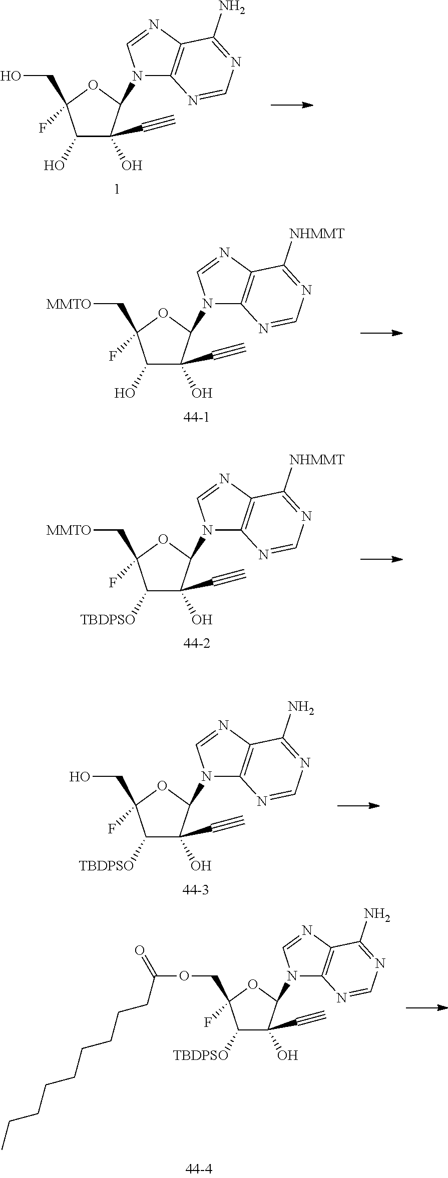

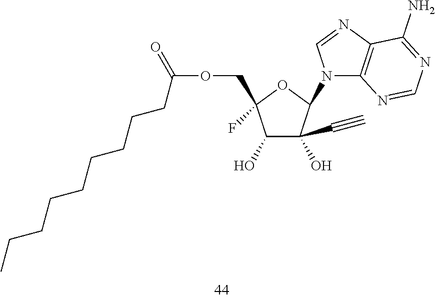 [SMILES: C#C[C@]1(O)[C@H](n2cnc3c(N)ncnc32)O[C@](F)(COC(=O)CCCCCCCCC)[C@H]1O]

To a solution of compound 1 ((2S,3S,4R,5R)-5-(6-amino-9H-purin-9-yl)-4-ethynyl-2-fluoro-2-(hydroxymethyl)tetrahydrofuran-3,4-diol, 300 mg, 0.9 mmol) in pyridine was added MMTCl (095 g, 3.0 mmol) and the resulting mixture stirred at R.T. for 1 d. An additional portion of MMTCl (0.16 g, 0.5 mmol) was added and stirring continued at 40° C. for 2 h. After cooling to R.T., the reaction was quenched with MeOH and the mixture concentrated and coevaporated with toluene. The residue was partitioned between water and EA. The organic layer was washed with sat. aq. NaHCO₃ and brine and dried (Na₂SO₄). After concentrating under reduced pressure, the residue was applied onto a silica gel column with EA/Hexanes (2:10 to 1:0) to provide 0.66 g (87%) 44-1 ((2S,3S,4R,5R)-4-ethynyl-2-fluoro-2-(((4-methoxyphenyl)diphenylmethoxy)methyl)-5-(6-(((4-methoxyphenyl)diphenylmethyl)amino)-9H-purin-9-yl)tetrahydrofuran-3,4-diol).

A mixture of 44-1 (0.51 g, 0.6 mmol), imidazole (82 mg, 1.2 mmol), TBDPSCl (0.16 mL, 0.6 mmol) and DMAP (7 mg, 0.06 mmol) in DCM (7 mL) was stirred for 1 d at R.T. Additional amounts of imidazole (82 mg, 1.2 mmol), TBDPSCl (0.16 mL, 0.6 mmol) and DMAP (7 mg, 0.06 mmol) were added and stirring continued for 12 h. The mixture was then diluted with EA and washed with 1N citric acid, water, sat. aq. NaHCO₃ and brine and dried (Na₂SO₄). Purification on silica gel with EA/Hex (1:10 to 8:10) yielded 0.52 g (80%) 44-2 ((2R,3R,4S,5S)-4-((tert-butyldiphenylsilyl)oxy)-3-ethynyl-5-fluoro-5-(((4-methoxyphenyl)diphenylmethoxy)methyl)-2-(6-(((4-methoxyphenyl)diphenylmethyl)amino)-9H-purin-9-yl)tetrahydrofuran-3-ol).

44-2 (0.62 g, 0.57 mmol) was treated in 80% aq. Formic acid for 1 h. The mixture was evaporated and the residue coevaporated with toluene/ACN. The residue was applied onto a silica gel column with MeOH/DCM (3:100 to 10:100) provided 0.28 g (86%) of 44-3 ((2R,3R,4S,5S)-2-(6-amino-9H-purin-9-yl)-4-((tert-butyldiphenylsilyl)oxy)-3-ethynyl-5-fluoro-5-(hydroxymethyl)tetrahydrofuran-3-ol).

A mixture of 44-3 (208 mg, 0.38 mmol) in pyridine (4 mL) and decanoic anhydride (0.25 g, 0.76 mmol) was stirred for 12 h at R.T., then coevaporated with toluene. Purification on silica gel with MeOH/DCM (3:100 to 10:100) provided 74 mg (38%) 44-4 (((2S,3S,4R,5R)-5-(6-amino-9H-purin-9-yl)-3-((tert-butyldiphenylsilyl)oxy)-4-ethynyl-2-fluoro-4-hydroxytetrahydrofuran-2-yl)methyldecanoate).

To an ice-cold solution of 44-4 (74 mg, 0.1 mmol) in THF (2 mL) was added TBAF (1.0 M in THF, 0.2 mL, 0.2 mmol) and the mixture allowed to warm to R.T. After 30 mins the reaction was quenched with silica, evaporated and purified on silica gel with iPrOH/DCM (3:100 to 15:100), providing 37 mg (80%) of 44 (((2S,3S,4R,5R)-5-(6-amino-9H-purin-9-yl)-4-ethynyl-2-fluoro-3,4-dihydroxytetrahydrofuran-2-yl)methyl decanoate). ¹H-NMR (CD₃CN): δ 8.25, 8.02 (2 s, 2H, H-2, H-8), 6.40 (s, 1H, H-1′), 6.16 (br s, 2H, NH₂), 5.08 (d, J=18.4 Hz, 1H, H-3′), 4.55 (dd, J=10.2 Hz, 12.2 Hz, 1H, H-5′a), 4.42 (app t, J=11.7 Hz, H-5′b), 2.51 (s, 1H, C≡CH), 2.37 (m, 2H, C(O)CH₂), 1.57 (m, 2H, CH₂), 1.26 (m, 12H, (CH₂)₆CH₃), 0.73 (m, 3H, CH₃), 0.95 (t, J=7.2 Hz, 3H, CH₃). ¹⁹F-NMR (CD₃CN): δ−120.89 (m). MS m/z=464.05 [M+1]⁺.

### Example 31

### Compound 46: ((2R,3R,4R,5R)-2-(6-amino-9H-purin-9-yl)-3-ethynyl-5-(hydroxymethyl)tetrahydrofuran-3,4-diol)

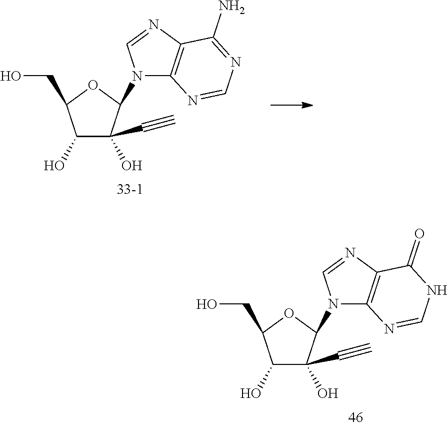 [SMILES: C#C[C@@]1(O)[C@H](O)[C@@H](CO)O[C@H]1n1cnc2c(N)ncnc21 ; C#C[C@@]1(O)[C@H](O)[C@@H](CO)O[C@H]1n1cnc2c(=O)[nH]cnc21]

Compound 46 was prepared from compound 33-1 ((2R,3R,4R,5R)-2-(6-amino-9H-purin-9-yl)-3-ethynyl-5-(hydroxymethyl)tetrahydrofuran-3,4-diol, 115 mg, 0.4 mmol) in glacial acetic acid (2 mL) with 4 M aq. solution of NaNO₂ (4×200 μL, 4×0.8 mmol) in the a manner analogous to compound 30 from compound 1. Purification by reverse phase HPLC (0-30% B; A: 50 mM aq. triethylammonium acetate (TEAA), B: 50 mM TEAA in ACN) gave 46 (50 mg, 42%). ¹H-NMR (DMSO-d₆): δ 12.3 (br, 1H, NH), 8.36, 8.03 (2 s, 2H, H-2, H-8), 5.97 (s, 1H, H-1′), 6.4, 5.8, 5.2 (3 br, 3×1H, 3OH), 4.34 (d, J=8.8 Hz, 1H, H-3′), 3.86 (m, 1H, H-4′), 3.77, 3.63 (2 m, 2H, H-5′a, H-5′b), 3.13 (s, 1H, C≡CH), MS m/z=291.3.0 (M−1).

### Example 32

### Compound 47: (2S,3R,4R,5M)-2-(4-aminopyrazolo[1,5-a][1,3,5]triazin-8-yl)-3-ethynl-5-(hydroxymethyl)tetrahydrofuran-3,4-diol

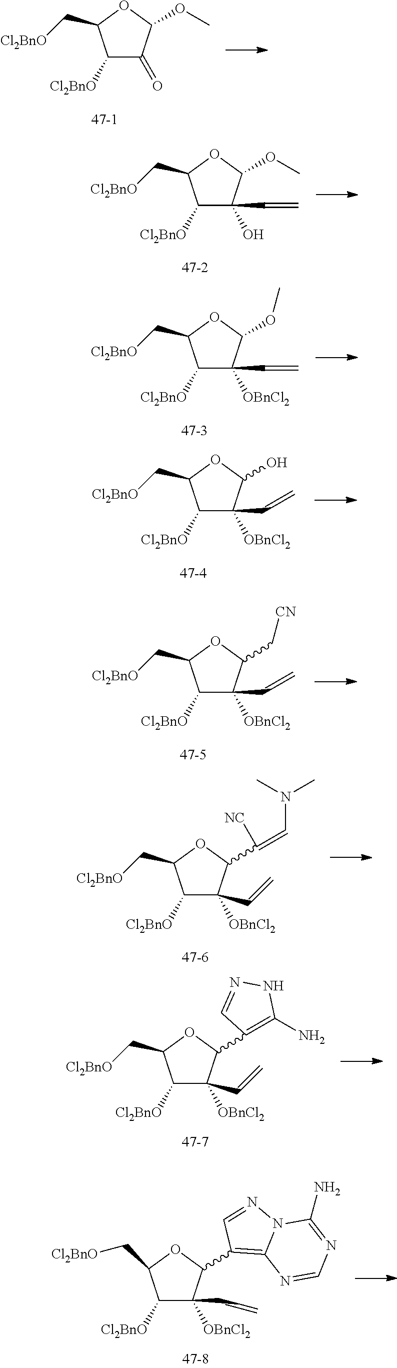

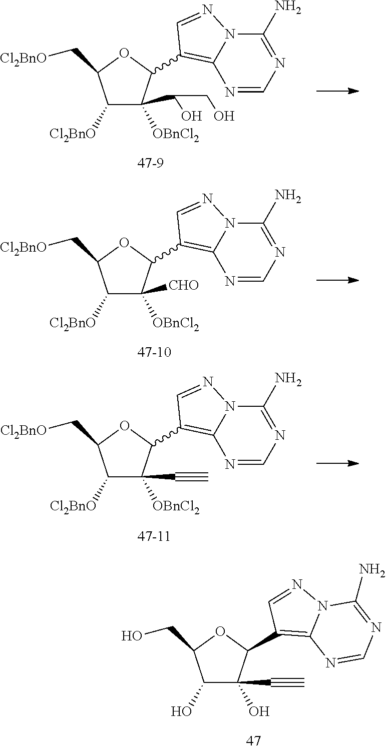

To a solution of 47-1 ((2S,4R,5R)-4-((2,4-dichlorobenzyl)oxy)-5-(((2,4-dichlorobenzyl)oxy)methyl)-2-methoxydihydrofuran-3(2H)-one, 13.0 g, 27.1 mmol) in THF (150 mL) was added bromo(vinyl)magnesium (1 M, 54.1 mL) dropwise at −78° C. The mixture was stirred at 20° C. for 3 h. The mixture was poured into saturated NH₄Cl solution (100 mL) and extracted twice with EA (100 mL) and washed with brine (100 mL). After concentrating under reduced pressure, the residue was applied onto a silica gel column with PE/EA (40:1 to 10:1) to give 47-2 ((2S,3R,4R,5R)-4-((2,4-dichlorobenzyl)oxy)-5-(((2,4-dichlorobenzyl)oxy)methyl)-2-methoxy-3-vinyltetrahydrofuran-3-ol, 24 g, 42.31 mmol, 78.15%, 89.6% purity) as a yellow oil. LCMS: ESI -MS: m/z=530.8 [M+Na]⁺.

To a solution of 47-2 (12.0 g, 23.6 mmol, two batches) in DMF (200 mL) was added NaH (1.42 g, 35.4 mmol) at 0° C. The mixture was stirred at 0° C. for 1 h and 2,4-dichloro-1-(chloromethyl)benzene (6.92 g, 35.4 mmol) and TBAI (1.74 g, 4.7 mmol) were added. The mixture was stirred at 25° C. for 1 h. The reaction was quenched by addition of saturated NH₄Cl solution (100 mL) and then diluted with EA (50 mL) and extracted with EA (50 mL×3). The combined organic layers were washed with saturated brine (20 mL×2) and dried over Na₂SO₄. After concentrating under reduced pressure, the residue was on silica gel with PE/EA (30:1 5:1) to give 47-3 ((2S,3R,4R,5R)-3,4-bis((2,4-dichlorobenzyl)oxy)-5-(((2,4-dichlorobenzyl)oxy)methyl)-2-methoxy-3-vinyltetrahydrofuran, 31.5 g, 47.2 mmol, 100%, 100% purity) as a yellow oil. LCMS: ESI-MS: m/z=688.8 [M+Na]⁻.

To a solution of 47-3 (15 g, 22.5 mmol) in AcOH (200 mL) was added water (10.0 g, 555 mmol, 10 mL) and H₂SO₄ (8.82 g, 89.9 mmol, 4.79 mL). The mixture was stirred at 105° C. for 5 h. The mixture was diluted with EA (300 mL) and extracted with EA (50 mL×3). The combined organic layers were washed with a saturated solution of NaHCO₃ (200 mL×2) and dried over Na₂SO₄. After concentrating under reduced pressure, the residue was purified on silica gel with PE/EA (30:1 to 5:1) to give 47-4 ((3R,4R,5R)-3,4-bis((2,4-dichlorobenzyl)oxy)-5-(((2,4-dichlorobenzyl)oxy)methyl)-3-vinyltetrahydrofuran-2-ol, 12 g, 18.4 mmol, 81.7%) as a colorless oil. LCMS: ESI-MS: m/z=674.8, 676.8 [M+Na]⁺.

To a solution of NaH (119 mg, 3 mmol) in DME (10 mL) was added 2-diethoxyphosphorylACN (705 mg, 4 mmol, 640 μL) and stirred at 0° C. for 30 min. 47-4 (1.3 g, 2 mmol in DME (10 mL) was added. The mixture was stirred at 0-25° C. for 2 h. The mixture was quenched with H₂O (10 mL) and extracted with EA (20 mL×2). The combined organic layers were dried over Na₂SO₄, filtered and concentrated under reduced pressure. The residue was purified by flash silica gel chromatography (ISCO®; 12 g SepaFlash® Silica Flash Column, Eluent of 5˜12% Ethylacetate/Petroleum ether gradient @ 28 mL/min) to give 47-5 (2-((3S,4R,5R)-3,4-bis((2,4-dichlorobenzyl)oxy)-5-(((2,4-dichlorobenzyl)oxy)methyl)-3-vinyltetrahydrofuran-2-yl)ACN, 2.4 g, 3.3 mmol, 82%) as a colorless oil. ESI-MS: m/z=674.0 [M+H]⁻, 697.9 [M+Na]⁻

To a solution of 47-5 (2.6 g, 3.8 mmol) in DMF (25 mL) was added 1-tert-butoxy-N,N,N′,N′-tetramethyl-methanediamine (3.35 g, 19.2 mmol, 4 mL). The mixture was stirred at 60° C. for 12 h. The mixture was quenched with H₂O (15 mL) and extracted with EA (20 mL×2) and the combined organic layers were dried over Na₂SO₄. After concentrating under reduced pressure, the residue purified by flash silica gel chromatography (ISCO®; 24 g SepaFlash® Silica Flash Column, Eluent of 5-50% Ethyl acetate/Petroleum ether gradient @ 35 mL/min) to give 47-6 (2-((3S,4R,5R)-3,4-bis((2,4-dichlorobenzyl)oxy)-5-(((2,4-dichlorobenzyl)oxy)methyl)-3-vinyltetrahydrofuran-2-yl)-3-(dimethylamino)acrylonitrile, 5 g 6.7 mmol, 87.1%) as colorless oil. LCMS: ESI-MS: m/z=752.8 [M+Na]⁺.

To a solution of 47-6 (2.5 g, 3.4 mmol) in EtOH (20 mL) and H₂O (4 mL) was added hydrazine (1.87 g, 27.4 mmol). The mixture was stirred at 105° C. for 2 h. The mixture was quenched with NaHCO₃ (10 mL) and extracted with EA (20 mL×2) and the combined organic layers were dried over Na₂SO₄. After concentrating under reduced pressure, the residue purified by flash silica gel chromatography (ISCO®; 12 g SepaFlash® Silica Flash Column, Eluent of 10×100% Ethyl acetate/Petroleum ether gradient @ 30 mL/min) to give 47-7 (4-((3S,4R,5R)-3,4-bis((2,4-dichlorobenzyl)oxy)-5-(((2,4-dichlorobenzyl)oxy)methyl)-3-vinyltetrahydrofuran-2-yl)-1H-pyrazol-5-amine, 4.2 g, 5.8 mmol, 84.7%) as colorless oil. LCMS: ESI-MS: m/z=739.6, 741.8 [M+Na]⁺.

To a solution of 47-7 (1.7 g, 2.4 mmol) in toluene (20 mL) was added ethyl(Z)-N-cyanomethanimidate (2.1 g, 21.3 mmol). The mixture was stirred at 85° C. for 2.5 h. After concentrating under reduced pressure, the residue purified by flash silica gel chromatography (ISCO®; 12 g SepaFlash® Silica Flash Column, Eluent of 10˜100% Ethyl acetate/Petroleum ether gradient @ 30 mL/min) to give 47-8 (8-((3S,4R,5R)-3,4-bis((2,4-dichlorobenzyl)oxy)-5-(((2,4-dichlorobenzyl)oxy)methyl)-3-vinyltetrahydrofuran-2-yl)pyrazolo[1,5-a][1,3,5]triazin-4-amine, 2.7 g, 3.5 mmol, 74%) as a yellow foam. LCMS: ESI-MS: m/z=769.8, 769.9 [M+H]⁺.

To a solution of compound 47-8 (0.85 g, 1.1 mmol) in THF (8 mL) was added OsO₄ (0.1 M, 3.3 mL), NMO (194 mg, 1.7 mmol, 175 μL) and H₂O (1.2 mL). The mixture was stirred at 30° C. for 12 h, then quenched with Na₂S₂O₄ (4 mL) and extracted with EA (8 mL×2) and the combined organic layers were dried over Na₂SO₄. After concentrating under reduced pressure, the residue purified by flash silica gel chromatography (ISCO®, 12 g SepaFlash® Silica Flash Column, Eluent of 0˜2% MeOH/DCM gradient @ 28 mL/min) to give 47-9 ((R)-1-((3S,4R,5R)-2-(4-aminopyrazolo[1,5-a][1,3,5]triazin-8-yl)-3,4-bis((2,4-dichlorobenzyl)oxy)-5-(((2,4-dichlorobenzyl)oxy)methyl)tetrahydrofuran-3-yl)ethane-1,2-diol, 1.1 g, 1.4 mmol, 62%) as a yellow foam. LCMS: ESI-MS: m/z=825.5, 825.6 [M+Na]⁻.

To a solution of 47-9 (0.8 g, 995 μmol) in H₂O (2.25 mL), MeOH (12.75 mL) and THF (3.75 mL) was added NaIO₄ (319 mg, 1.5 mmol, 83 μL). The mixture was stirred at 25° C. for 2 h. The mixture was quenched with Na₂SO₃ (5 mL) and extracted with EA (10 mL). The organic layer was dried with Na₂SO₄, filtered and concentrated under reduced pressure to give 47-10 ((3S,4R,5R)-2-(4-aminopyrazolo[1,5-a][1,3,5]triazin-8-yl)-3,4-bis((2,4-dichlorobenzyl)oxy)-5-(((2,4-dichlorobenzyl)oxy)methyl)tetrahydrofuran-3-carbaldehyde, 1.5 g, 1.9 mmol, 97.64%) a brown solid. LCMS: ESI-MS: m/z=793.7 [M+Na]⁺.

To a solution of K₂CO₃ (1.61 g, 11.7 mmol) and TsN₃ (766 mg, 3.9 mmol) in ACN (5 mL) was added 1-dimethoxyphosphorylpropan-2-one (645 mg, 3.9 mmol, 533 μL) at 25° C. under N₂. The mixture was stirred at 25° C. for 2 h. 47-10 (1.5 g, 1.9 mmol) in MeOH (5 mL) and ACN (5 mL) was added. The mixture was stirred at 25° C. for 12 h. The mixture was quenched with H₂O (5 mL) and extracted with EA (15 mL) and the organic layer was dried over Na₂SO₄. After concentrating under reduced pressure, the residue was applied onto a silica gel column with to give 47-11 (8-((3S,4R,5R)-3,4-bis((2,4-dichlorobenzyl)oxy)-5-(((2,4-dichlorobenzyl)oxy)methyl)-3-ethynyltetrahydrofuran-2-yl)pyrazolo[1,5-a][1,3,5]triazin-4-amine, Beta-isomer, 0.5 g, 35.7%) as a brown solid. LCMS: m/z=767.6 [M+H]⁺.

To a solution of 47-11 (0.2 g, 260 μmol) in DCM (2 mL) was added BCl₃ (1 M, 2.6 mL) at −78° C. The mixture was stirred at 0° C. for 2 h. The mixture was quenched with MeOH (2 mL) and the solvent was removed. Two drops of NH₃H₂O was added in MeOH (2 mL). The mixture was stirred at 25° C. for 12 h. After concentrating under reduced pressure, the residue was applied onto a silica gel column with DCM/MeOH (50:1 to 15:1) to give 47 ((2S,3R,4R,5R)-2-(4-aminopyrazolo[1,5-a][1,3,5]triazin-8-yl)-3-ethynyl-5-(hydroxymethyl)tetrahydrofuran-3,4-diol. 35 mg, 118 μmol, 45.2%) as a white solid. ¹H NMR (400 MHz, CD₃OD) δ=8.24 (s, 1H), 8.06 (s, 1H), 5.22 (s, 1H), 4.28 (d, J=6.5 Hz, 1H), 4.02-3.90 (m, 2H), 3.86-3.77 (m, 1H), 2.82 (s, 1H). LCMS: ESI-MS: m/z=292.1 [M+H]⁺.

### Example 33

### Compound 48: tert-butyl(9-((2R,3R,4S,5S)-3-ethynyl-5-fluoro-3,4-dihydroxy-5-(hydroxymethyl)tetrahydrofuran-2-yl)-9H-purin-6-yl)carbamate

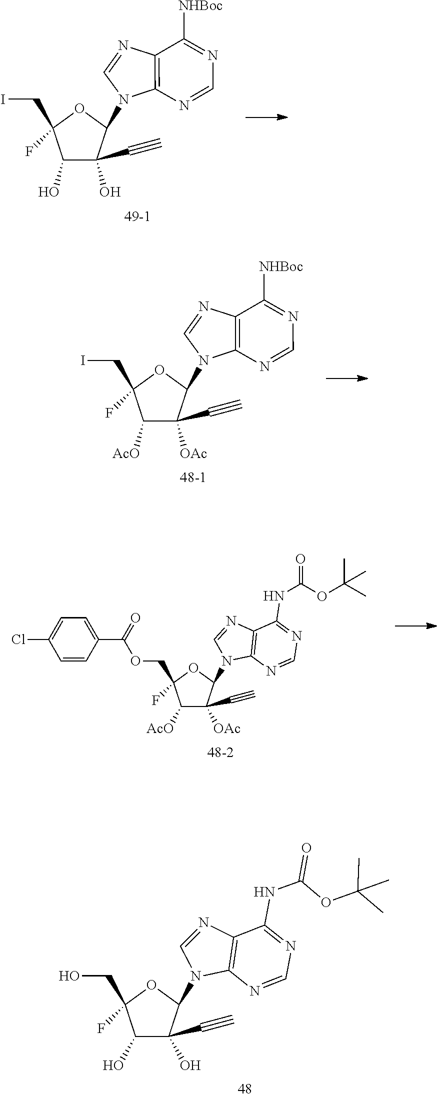 [SMILES: C#C[C@]1(O)[C@H](n2cnc3c(NC(=O)OC(C)(C)C)ncnc32)O[C@](F)(CI)[C@H]1O ; C#C[C@]1(OC(C)=O)[C@H](n2cnc3c(NC(=O)OC(C)(C)C)ncnc32)O[C@](F)(CI)[C@H]1OC(C)=O ; C#C[C@]1(OC(C)=O)[C@H](n2cnc3c(NC(=O)OC(C)(C)C)ncnc32)O[C@](F)(COC(=O)c2ccc(Cl)cc2)[C@H]1OC(C)=O ; C#C[C@]1(O)[C@H](n2cnc3c(NC(=O)OC(C)(C)C)ncnc32)O[C@](F)(CO)[C@H]1O]

To a solution of 49-1 (2′-C-ethynyl-4′-fluoro-5′-deoxy-5′-iodo-6-N-Boc-adenosine, 14.65 g, 28.21 mmol) in 460 mL of anhydrous acetonitrile was added triethylamine (22.8 g, 8 eq.) at 0° C. followed by 40 mg of DMAP. Acetic anhydride (5.9 g, 2 eq.) was added dropwise to form a clear solution. The reaction was stirred at R.T. and completed in 2 h. After quenching with methanol, the mixture was concentrated under reduced pressure. The residue was purified via column chromatography (silica gel, 0-30% EtOAc in DCM) to afford 48-1 (2R,3S,4R,5R)-5-(6-((tert-butoxycarbonyl)amino)-9H-purin-9-yl)-4-ethynyl-2-fluoro-2-(iodomethyl)tetrahydrofuran-3,4-diyldiacetate) as a white solid (69%). LC-MS: 604 [M+1]⁺.

Compound 48-1 (2R,3S,4R,5R)-5-(6-((tert-butoxycarbonyl)amino)-9H-purin-9-yl)-4-ethynyl-2-fluoro-2-(iodomethyl)tetrahydrofuran-3,4-diyldiacetate, 16.96 g, 28.1 mmol) was added to a stirred mixture of tetra-n-butylammonium hydrogensulfate (10.5 g, 31 mmol), di-potassium hydrogenphosphate (14.7 g, 84 mmol) and m-chlorobenzoic acid (11 g, 70 mmol) in DCM and water. M-chloroperbenzoic acid (˜70%, 19.4 g, 112 mmol) was then added. The mixture was stirred at R.T. for overnight, and the reaction was quenched by the addition of a solution of sodium sulphite (Na₂SO₃, 17 g, 135 mmol) in water (85 mL). After aqueous work-up and column chromatography, 48-2 (2S,3S,4R,5R)-5-(6-((tert-butoxycarbonyl)amino)-9H-purin-9-yl)-2-(((4-chlorobenzoyl)oxy)methyl)-4-ethynyl-2-fluorotetrahydrofuran-3,4-diyldiacetate) was collected as a colorless oil (80%). LC-MS: 632 [M+1]⁺.

A mixture of 48-2 ((2S,3S,4R,5R)-5-(6-((tert-butoxycarbonyl)amino)-9H-purin-9-yl)-2-(((4-chlorobenzoyl)oxy)methyl)-4-ethynyl-2-fluorotetrahydrofuran-3,4-diyldiacetate, 0.16 g, 0.25 mmol) in BuNH₂ (1 mL) was stirred at R.T. for 30 min. After concentrating under reduced pressure, the residue was purified on silica gel with MeOH/DCM (4:100-15:100) to provide 90 mg (88%) of 48 (tert-butyl(9-((2R,3R,4S,5S)-3-ethynyl-5-fluoro-3,4-dihydroxy-5-(hydroxymethyl)tetrahydrofuran-2-yl)-9H-purin-6-yl)carbamate). ¹H-NMR (DMSO-d₆): δ 10.14 (s, 1H, NH), 8.60, 8.55 (2 s, 2H, H-2, H-8), 6.58 (s, 1H, H-1′), 6.43 (s, 1H, 2′—OH), 6.02 (d, J=8.8 Hz, 1H, OH—3′), 5.69 (t, J=6.0 Hz 1H, OH—5′), 4.67 (dd, J=9.2 Hz, 19.6 Hz, 1H, H—3′), 3.66 (m, 2H, H—5′a, H—5′b), 3.15 (s, 1H, C≡CH), 1.44 (s, 9H, CMe₃). ¹⁹F-NMR (DMSO-d₆): δ−120.73 (m). MS m/z=409.95 [M+1]⁺.

### Example 34

### Compound 49: (2S,3S,4S,5R)-5-(6-amino-9H-purin-9-yl)-4-ethynyl-2-fluoro-4-hydroxy-2-(hydroxymethyl)tetrahydrofuran-3-ylpropylcarbamate

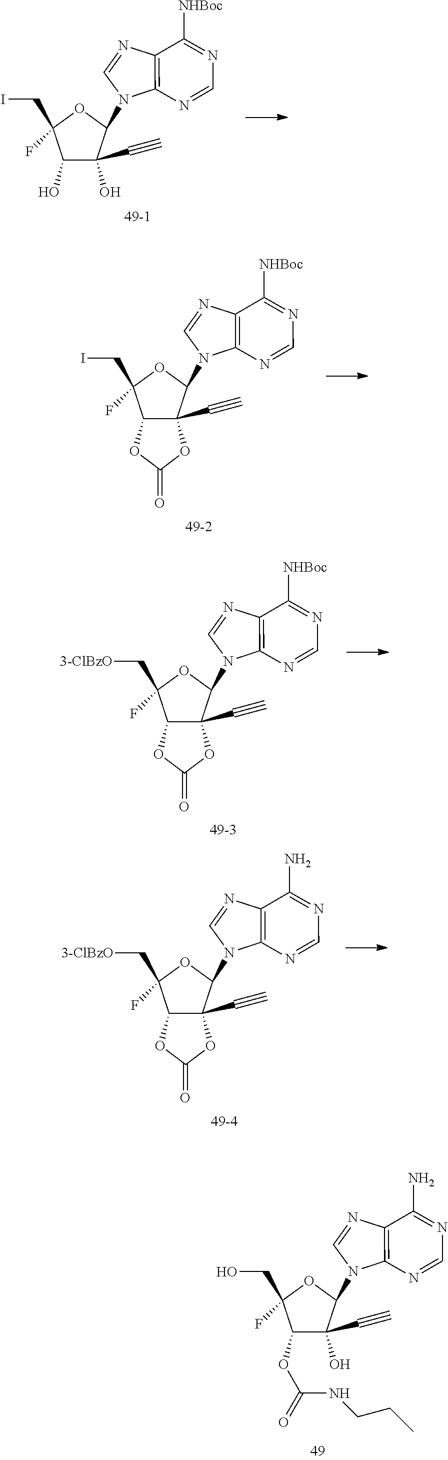

To a solution of 49-1 (tert-butyl(9-((2R,3R,4S,5R)-3-ethynyl-5-fluoro-3,4-dihydroxy-5-(iodomethyl)tetrahydrofuran-2-yl)-9H-purin-6-yl)carbamate, 1.06 g, 2 mmol) in 10 mL of anhydrous DMF was added 662 mg of CDI (4.1 mmol) at 0° C. The mixture was stirred at R.T. for 2 h and then quenched by addition of water. After aqueous work-up and column chromatography, 49-2 was collected as a white solid (tert-butyl(9-((3aR,4R,6R,6aS)-3a-ethynyl-6-fluoro-6-(iodomethyl)-2-oxotetrahydrofuro[3,4-d][1,3]dioxol-4-yl)-9H-purin-6-yl)carbamate, 380 mg, 34%). LC-MS: 546 [M+1]⁺.

49-2 (380 mg, 0.7 mmol) was added to a stirred mixture of tetra-n-butylammonium hydrogensulfate (260 mg, 0.8 mmol), K₂HPO₄. (366 mg, 2.1 mmol) and m-chlorobenzoic acid (274 mg, 1.8 mmol) in DCM and water. m-Chloroperbenzoic acid (70%, 485 mg, 2.8 mmol) was added. The reaction was stirred at R.T. for overnight and quenched by addition of a solution of sodium sulphite (Na₂SO₃, 675 mg, 5.3 mmol) in water (4 mL). After aqueous work-up and column chromatography, 49-3 was collected as a foamy solid (((3aS,4S,6R,6aR)-6-(6-((tert-butoxycarbonyl)amino)-9H-purin-9-yl)-6a-ethynyl-4-fluoro-2-oxotetrahydrofuro[3,4-d][1,3]dioxol-4-yl)methyl2-(3-chlorophenyl)acetate, 176 mg, 58%). LC-MS: 574 [M+1]⁺.

To a solution of 49-3 (176 mg, 0.3 mmol) in anhydrous DCM (5 mL) was added 0.6 mL of TFA and the mixture was stirred at R.T. for 3 h. After removal of solvent, the residue was co-evaporated with 2-propanol three times to afford a foamy crude 49-4 (((3aS,4S,6R,6aR)-6-(6-amino-9H-purin-9-yl)-6a-ethynyl-4-fluoro-2-oxotetrahydrofuro[3,4-d][1,3]dioxol-4-yl)methyl2-(3-chlorophenyl)acetate) which was used directly in next step. To the crude 49-4 was added n-propylamine (840 mg) at 0° C. and the mixture was stirred at R.T. for 2 h. After removal of propylamine under reduced pressure, 49 was isolated via column chromatography as a white powder ((2S,3S,4R,5R)-5-(6-amino-9H-purin-9-yl)-4-ethynyl-2-fluoro-4-hydroxy-2-(hydroxymethyl)tetrahydrofuran-3-ylpropylcarbamate, 95 mg, 78%). ¹H NMR (dmso-d₆) d (ppm): 8.32 (s, 1H), 8.17 (s, 1H), 7.54 (t, 1H), 7.36 (s, 2H), 6.89 (s, 1H), 6.42 (s, 1H), 5.90 (d, 1H), 5.67 (t, 1H), 3.72-3.61 (m, 2H), 3.01-2.97 (m, 2H), 1.49-1.40 (m, 2H), 0.84 (t, 3H); LC-MS: 395 [M+1]⁺.

### Example A

### Picornavirus Assay

HeLa-OHIO cells (Sigma-Aldrich, St. Louis, Mo.) were plated in 96 well plates at a density of 1.5×10⁵ cells per well in assay media (MEM without phenol red or L-glutamine, supplemented with 1% FBS, 1% penicillin/streptomycin, 2 mM GlutaGro and 1× MEM nonessential amino acids, all from Cellgro, Manassas, Va.). Assay setup took place after allowing cells to adhere for 24 h. Compounds dissolved in DMSO were serially diluted in assay media to 2× final concentration. Media was aspirated from the cells and 100 μl media with compound was added in triplicate. Human rhinovirus 1B (ATCC, Manassas, Va.) was diluted in assay media and 100 μL was added to cells and compound. The virus inoculum was selected to cause 80-90% cytopathic effect in 4 d. Infected cells were incubated for 4 d at 33° C., 5% CO₂. To develop the assay, 100 μL media was replaced with 100 μCellTiter-Glo® reagent (Promega, Madison, Wis.) and incubated for 10 mins at R.T. Luminescence was measured on a Victor ×3 multi-label plate reader.

HeLa-OHIO cells were plated at a density of 1.5×10⁵ cells per mL (1.5×10⁴ cells per well) in assay media (MEM without phenol red or L-glutamine (Gibco cat. #51200) supplemented with 1% FBS, 1% penicillin/streptomycin (Mediatech cat. #30-002-CI) and 1% Glutamax (Gibco cat. #35050) in clear-bottom black 96 well plates. After 24 h, media was removed and replaced with serially diluted compounds in assay media. For EC₅₀ measurements, cells were infected with HRV-1b or an equivalent inoculum for the other virus strains in 100 μL assay media. The virus inoculum was selected to cause 80-90% cytopathic effect in 4-6 d. After 4-6 days, cell viability was measured using CellTiter Glo Luminescent Cell Viability Assay (Promega cat. #G7572). 100 μL media was removed from each well and 100 μL CellTiter Glo reagent was added. Plates were incubated at R.T. for 5 mins, then luminescence was measured using a Perkin Elmer multilabel counter Victor3V. EC₅₀ values were determined using XLFit.

### Example B

### Picornavirus Polymerase Inhibition Assay

The enzyme activity of human rhinovirus 16 polymerase (HRV16pol) was measured as an incorporation of tritiated NMP into acid-insoluble RNA products. hV16pol assay reactions contained 30 Nm recombinant enzyme, 50 nM heteropolymeric RNA, about 0.5 μCi tritiated NTP, 0.1 mM of competing cold NTP, 40 mM Tris-HCl (pH 7.0), 3 Mm dithiothreitol and 0.5 mM MgCl₂. Standard reactions were incubated for 2.5 h at 30° C., in the presence of increasing concentration of inhibitor. At the end of the reaction, RNA was precipitated with 10% TCA and acid-insoluble RNA products were filtered on a size exclusion 96-well plate. After washing of the plate, scintillation liquid was added and radiolabeled RNA products were detected using standard procedures with a Trilux Microbeta scintillation counter. The compound concentration at which the enzyme-catalyzed rate was reduced by 50% (IC₅₀) was calculated by fitting the data to a non-linear regression (sigmoidal).

### Example C

### Enterovirus Assay

### Cells

HeLa OHIO cells were purchased from Sigma Aldrich (St Louis, Mo.) and cultured in MEM with Glutamax (Gibco cat. #41090) supplemented with 10% FBS (Mediatech cat. #35-011-CV) and 1% penicillin/streptomycin (Mediatech cat. #30-002-CI), at 37° C. with 5% CO₂. RD cells were purchased from ATCC (Manassas, Va.) and cultured in DMEM, supplemented with 10% FBS (Mediatech cat. #35-011-CV) and 1% penicillin/streptomycin (Mediatech cat. #30-002-CI), at 37° C. with 5% CO₂.

### Determination of Anti-Enterovirus Activity

For hV1b, hV14, hV16, hV75, EV68 and CVB3, HeLa-OHIO cells were plated at a density of 1.5×10⁵ cells per mL (1.5×10⁴ cells per well) in assay media (MEM without phenol red or L-glutamine (Gibco cat. #51200) supplemented with 1% FBS, 1% penicillin/streptomycin (Mediatech cat. #30-002-CI) and 1% Glutamax (Gibco cat. #35050)) in clear-bottom 96 well plates. For EV71, RD cells were plated at a density of 5×10⁴ cells per mL (5000 cells per well) in assay media (DMEM supplemented with 2% FBS and 1% penicillin/streptomycin). After 24 h, media was removed and replaced with serially diluted compounds in assay media. For EC₅₀ measurements, cells were infected in 100 μL assay media with a virus inoculum sufficient to obtain to cause 80-90% cytopathic effect. After 2-6 days, cell viability was measured using CellTiter Glo Luminescent Cell Viability Assay (Promega cat. #G7572). Cells infected with EV-71, EV-68 and CVB3 were cultured at 37° C., while cells infected with hV1b, hV-16, hV-75 were cultured at 33° C. 100 μL media was removed from each well and 100 μL CellTiter Glo reagent was added. Plates were incubated at R.T. for 5 mins, then luminescence was measured using a Perkin Elmer multilabel counter Victor3V. EC₅₀ values were determined using XLFit.

### Example D

### Dengue and Zika Viral Assay

The Dengue virus type 2 strain New Guinea C (NG-C) and the Dengue virus type 4 strain H241 were purchased from ATCC (Manassas, Va.; item numbers VR-1584 and VR-1490, respectively). The Zika virus strain MR766 was purchased from ATCC (item #VR-1838) and the Zika virus strain IbH 30656 was purchased from BEI Resources (Manassas, Va.; item number NR-500066). 24 h prior to dosing, Huh-7.5 cells were plated in 96 well plates at a density of 1.5×10⁵/mL in DMEM medium supplemented with 10% fetal bovine serum, 1% HEPES buffer, 1% Penicillin/Streptomycin and 1% non-essential amino acids (all Mediatech, Manassas, Va.). At the day of infection, serially diluted compounds were added to cells and incubated for 24 h. After the end of the 24 h pre-incubation period, cells were infected with either Dengue virus type 2 NG-C, Dengue virus type 4 H241, Zika virus strain MR766 or Zika virus strain IbH 30656. The virus inoculum was selected to cause 80-90% cytopathic effect in four (Zika) to five (Dengue) days. Infected cells were incubated for four to five days at 37° C., 5% CO₂. To develop the assay, 100 μL media was replaced with 100 μl CellTiter-Glo® reagent (Promega, Madison, Wis.) and incubated for 10 mins at R.T. Luminescence was measured on a Victor ×3 multi-label plate reader. Potential compound cytotoxicity was determined using uninfected parallel cultures.

### Example E

### HCV Replicon Assay

### Cells

Huh-7 cells containing the self-replicating, subgenomic HCV replicon with a stable luciferase (LUC) reporter were cultured in Dulbecco's modified Eagle's medium (DMEM) containing 2 mM L-glutamine and supplemented with 10% heat-inactivated fetal bovine serum (FBS), 1% penicillin-streptomyocin, 1% nonessential amino acids and 0.5 mg/Ml G418.

### Determination of Anti-HCV Activity

Determination of 50% inhibitory concentration (EC₅₀) of compounds in HCV replicon cells were performed by the following procedure. On the first day, 5000 HCV replicon cells were plated per well in a 96-well plate. On the following day, test compounds were solubilized in 100% DMSO to 100× the desired final testing concentration. Each compound was then serially diluted (1:3) up to 9 different concentrations. Compounds in 100% DMSO are reduced to 10% DMSO by diluting 1:10 in cell culture media. The compounds were diluted to 10% DMSO with cell culture media, which were used to dose the HCV replicon cells in 96-well format. The final DMSO concentration was 1%. The HCV replicon cells were incubated at 37° C. for 72 h. At 72 h, cells were processed when the cells are still subconfluent. Compounds that reduce the LUC signal are determined by Bright-Glo Luciferase Assay (Promega, Madison, Wis.). % Inhibition was determined for each compound concentration in relation to the control cells (untreated HCV replicon) to calculate the EC₅₀.

### Example F

### NS5B Inhibition Assay

The enzyme activity of NS5B-BK (Delta-21) was measured as an incorporation of tritiated NMP into acid-insoluble RNA products. The complementary IRES (cIRES) RNA sequence was used as a template, corresponding to 377 nucleotides from the 3′-end of HCV (-) strand. RNA, with a base content of 21% Ade, 23% Ura, 28% Cyt and 28% Gua. The cIRES RNA was transcribed in vitro using a T7 transcription kit (Ambion, Inc.) and purified using the Qiagen RNeasy maxi kit. HCV polymerase reactions contained 50 nM NS5B-BK, 50 nM cIRES RNA, about 0.5 μCi tritiated NTP, 1 μM of competing cold NTP, 20 mM NaCl, 40 mM Tris-HCl (pH 8.0), 4 mM dithiothreitol and 4 mM MgCl₂. Standard reactions were incubated for 2 h at 30° C., in the presence of increasing concentration of inhibitor. At the end of the reaction, RNA was precipitated with 10% TCA and acid-insoluble RNA products were filtered on a size exclusion 96-well plate. After washing of the plate, scintillation liquid was added and radio labeled RNA products were detected according to standard procedures with a Trilux Topcount scintillation counter. The compound concentration at which the enzyme-catalyzed rate was reduced by 50% (IC₅₀) was calculated by fitting the data to a non-linear regression (sigmoidal). The IC₅₀ values were derived from the mean of several independent experiments.

Compounds of Formulae (I) and (II) showed activity in one or more of the assays described above as summarized in Tables 4-6 below, where ‘A’ indicates an IC₅₀, EC₅₀<3 μM, ‘B’ indicates an IC₅₀, EC₅₀≥3 μM and <30 μM, ‘C’ indicates an IC₅₀, EC₅₀≥30 μM and <100 μM and “D” indicates an IC₅₀, EC₅₀≥100 μM.

TABLE 4

| | Viral Polymerase | | |
|---|---|---|---|
| | Inhibition IC₅₀ | | |
| Compound No. | Picornavirus | Dengue | HCV |
| 17 | A | B | A |
| 18 | A | B | A |
| 19 | A | B | A |
| 20 | A | B | A |
| 21 | A | C | A |
| 22 | A | B | A |
| 23 | A | A | A |
| 25 | B | B | A |
| 35 | D | A | A |
| 45 | A | A | A |
| 50 | A | A | B |
| 51 | A | A | A |
| 52 | A | A | A |
| 53 | A | B | A |
| 54 | C | C | A |
| 56 | A | B | A |
| 57 | A | A | A |
| 58 | A | A | A |

TABLE 5

| | | Compound No. | | | | | | | | | | | |
|---|---|---|---|---|---|---|---|---|---|---|---|---|---|
| Virus | | 1 | 2 | 3 | 4 | 6 | 7 | 10 | 13 | 15 | 16 | 26 | 43 |
| Dengue | EC₅₀ | A | A | A | D | B | A | B | D | D | D | A | A |
| NGC | | | | | | | | | | | | | |
| HCV | EC₅₀ | A | | | | | | | B | D | | | |
| (replicon) | | | | | | | | | | | | | |
| Zika | EC₅₀ | A | | | | | | | | | | | |
| HRV 1B | EC₅₀ | A | A | A | B | A | A | D | B | C | B | A | A |
| HRV 16 | EC₅₀ | A | | | | | | | | | | | |
| HRV 14 | EC₅₀ | A | | | | | | | | | | | |
| HRV 75 | EC₅₀ | A | | | | | | | | | | | |
| CVB3 | EC₅₀ | A | | | | | | | | | | | |
| EV71 | EC₅₀ | A | | | | | A | | | | | A | A |

*Dengue NGC—Dengue virus type 2 (NG-C strain), hV 1B—Human rhinovirus 1B, hV 16—Human rhinovirus 16, hV 14—Human rhinovirus 14, hV 75—Human rhinovirus 75 and CVB3—Coxsackie virus 3B

TABLE 6

| | | Compound No. | | | | | | | | | | | |
|---|---|---|---|---|---|---|---|---|---|---|---|---|---|
| Virus | | 27 | 31 | 34 | 36 | 37 | 38 | 39 | 40 | 41 | 42 | 44 | 49 |
| Dengue | EC₅₀ | A | A | A | A | A | A | A | A | A | A | A | C |
| NGC | | | | | | | | | | | | | |
| HRV 1B | EC₅₀ | A | A | A | A | A | A | A | A | A | A | A | B |

*Dengue NGC—Dengue virus type 2 (NG-C strain), hV 1B—Human rhinovirus LB

Although the foregoing has been described in some detail by way of illustrations and examples for purposes of clarity and understanding, it will be understood by those of skill in the art that numerous and various modifications can be made without departing from the spirit of the present disclosure. Therefore, it should be clearly understood that the forms disclosed herein are illustrative only and are not intended to limit the scope of the present disclosure, but rather to also cover all modification and alternatives coming with the true scope and spirit of the invention.

1. A compound of Formula (I), or a pharmaceutically acceptable salt thereof, having the structure: 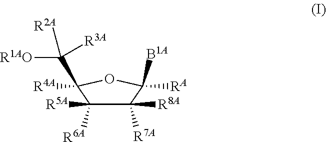
wherein:
B^{1A} is 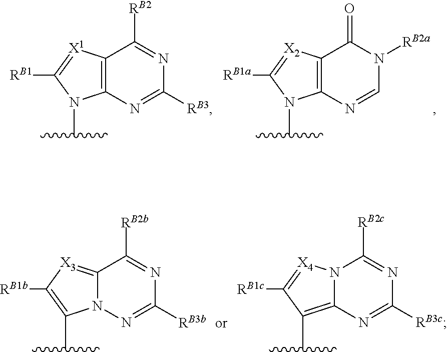
X¹ is N (nitrogen) or —CR^{B6};
X² is N (nitrogen) or —CR^{B6a};
X³ is N (nitrogen) or —CR^{B6b};
X⁴ is N (nitrogen) or —CR^{B6c};
R^{B1}, R^{B1a}, R^{B1b} and R^{B1c} are independently hydrogen or deuterium;
R^{B2} is NR^{B4a}R^{B4b};
R^{B2b} is NR^{B4a1}R^{B4b1};
R^{B2c} is NR^{B4a2}R^{B4b2};
R^{B2a} is selected from the group consisting of hydrogen, an optionally substituted C₁₋₆ alkyl, an optionally substituted C₂₋₆ alkenyl and an optionally substituted C₃₋₆ cycloalkyl;
R^{B3} is hydrogen, deuterium, halogen or NR^{B5a}R^{B5b};
R^{B3b} is hydrogen, deuterium, halogen or NR^{B5a1}R^{B5b1};
R^{B3c} is hydrogen, deuterium, halogen or NR^{B5a2}R^{B5b2};
R^{B4a}, R^{B4a1} and R^{B4a2} are independently hydrogen or deuterium;
R^{B4b}, R^{B4b1} and R^{B4b2} are independently selected from the group consisting of hydrogen, deuterium, an optionally substituted C₁₋₆ alkyl, an optionally substituted C₂₋₆ alkenyl, an optionally substituted C₃₋₆ cycloalkyl, —C(═O)R^{B7} and —C(═O)OR^{B8};
R^{B5a} is hydrogen or deuterium;
R^{B5b} is selected from the group consisting of hydrogen, an optionally substituted C₁₋₆ alkyl, an optionally substituted C₂₋₆ alkenyl, an optionally substituted C₃₋₆ cycloalkyl, —C(═O)R^{B9} and —C(═O)OR^{B10};
R^{B5a1} and R^{B5a2} are independently hydrogen or deuterium;
R^{B5b1} and R^{B5b2} are independently selected from the group consisting of hydrogen, an optionally substituted C₁₋₆ alkyl, an optionally substituted C₂₋₆ alkenyl, an optionally substituted C₃₋₆ cycloalkyl, —C(═O)R^{B9} and —C(═O)OR^{B10};
R^{B6a}, R^{B6b} and R^{B6c} are independently selected from the group consisting of hydrogen, deuterium, halogen, —C≡N, —C(═O)NH₂, an optionally substituted C₁₋₆ alkyl, an optionally substituted C₂₋₆ alkenyl and an optionally substituted C₂₋₆ alkynyl;
R^{B7}, R^{B8}, R^{B9} and R^{B10} are independently selected from the group consisting of an optionally substituted C₁₋₆ alkyl, an optionally substituted C₂₋₆ alkenyl, an optionally substituted C₂₋₆ alkynyl, an optionally substituted C₃₋₆ cycloalkyl, an optionally substituted C₅₋₁₀ cycloalkenyl, an optionally substituted C₆₋₁₀ aryl, an optionally substituted heteroaryl, an optionally substituted heterocyclyl, an optionally substituted aryl (C₁₋₆ alkyl), an optionally substituted heteroaryl (C₁₋₆ alkyl) and an optionally substituted heterocyclyl (C₁₋₆ alkyl);
R^{1A} is hydrogen, deuterium, an optionally substituted acyl, an optionally substituted O-linked amino acid or 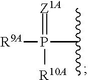
R^{2A}, R^{3A}, R^{5A} and R^{A} are independently hydrogen or deuterium;
R^{4A} is fluoro;
R^{6A} is selected from the group consisting of —OH, —OC(═O)R″^{A} and an optionally substituted O-linked amino acid;
R^{7A} is —OH, —OC(═O)R″^{B}, fluoro or chloro;
R^{8A} is an optionally substituted C₁₋₃ alkyl, an optionally substituted C₃₋₆ allenyl or an optionally substituted C₂₋₆ alkynyl;
R^{9A} and R^{10A} are independently selected from the group consisting of O⁻, —OH, an optionally substituted —O—C₁₋₂₄ alkyl, an optionally substituted —O—C₂₋₂₄ alkenyl, an optionally substituted —O—C₂₋₂₄ alkynyl, an optionally substituted —O—C₃₋₆ cycloalkyl, an optionally substituted —O—C₅₋₁₀ cycloalkenyl, an optionally substituted —O-aryl, an optionally substituted —O-heteroaryl, an optionally substituted —O-aryl (C₁₋₆ alkyl), an optionally substituted *—O—(CR^{11A}R^{12A})ₚ—O—C₁₋₂₄ alkyl, an optionally substituted *—O—(CR^{13A}R^{14A})_{q}—O—C₂₋₂₄ alkenyl, 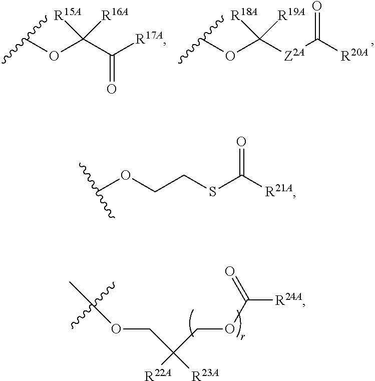
an optionally substituted N-linked amino acid and an optionally substituted N-linked amino acid ester derivative; or
R^{9A} is 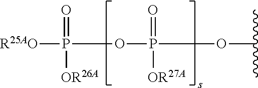
and R^{10A} is O⁻ or OH; or
R^{9A} and R^{10A} are taken together to form a moiety selected from an optionally substituted 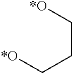
and an optionally substituted 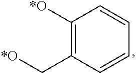
wherein the phosphorus and the moiety form a six-membered to ten-membered ring system and wherein the asterisks indicate the points of attachment of the moieties;
each R^{11A}, each R^{12A}, each R^{13A} and each R^{14A} are independently hydrogen, deuterium, an optionally substituted C₁₋₂₄ alkyl or alkoxy;
R^{15A}, R^{16A}, R^{18A} and R^{19A} are independently selected from the group consisting of hydrogen, deuterium, an optionally substituted C₁₋₂₄ alkyl and an optionally substituted aryl;
R^{17A} and R^{20A} are independently selected from the group consisting of hydrogen, deuterium, an optionally substituted C₁₋₂₄ alkyl, an optionally substituted aryl, an optionally substituted —O—C₁₋₂₄ alkyl, an optionally substituted —O-aryl, an optionally substituted —O-heteroaryl and an optionally substituted —O-monocyclic heterocyclyl;
R^{21A} is selected from the group consisting of hydrogen, deuterium, an optionally substituted C₁₋₂₄ alkyl and an optionally substituted aryl;
R^{22A} and R^{23A} are independently selected from the group consisting of —C≡N, an optionally substituted C₂₋₈ organylcarbonyl, an optionally substituted C₂₋₈ alkoxycarbonyl and an optionally substituted C₂₋₈ organylaminocarbonyl;
R^{24A} is selected from the group consisting of hydrogen, deuterium, an optionally substituted C₁₋₂₄-alkyl, an optionally substituted C₂₋₂₄ alkenyl, an optionally substituted C₂₋₂₄ alkynyl, an optionally substituted C₃₋₆ cycloalkyl and an optionally substituted C₅₋₁₀ cycloalkenyl;
R^{25A}, R^{26A} and R^{27A} are independently absent, hydrogen or deuterium;
p and q are independently selected from 1, 2 and 3;
r is 1 or 2;
s is 0 or 1;
R″^{A} and R″^{B} are independently an optionally substituted C₁₋₂₄ alkyl; and
Z^{1A} and Z^{2A} are independently oxygen (O) or sulfur (S); and
provided that when X¹ is N or CH, B^{1A} is 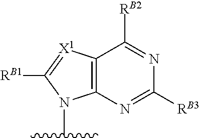
and R^{1A} is hydrogen or triphosphate, then R^{8A} is not methyl; and
provided that the compound of Formula (I) is not 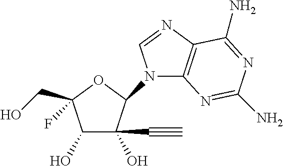 [SMILES: C#C[C@]1(O)[C@H](n2cnc3c(N)nc(N)nc32)O[C@](F)(CO)[C@H]1O]
or a pharmaceutically acceptable salt thereof.

2. (canceled)

3. The compound of claim 1, wherein R^{1A} is hydrogen or deuterium.

4. The compound of claim 1, wherein R^{1A} is an optionally substituted acyl.

5. The compound of claim 4, wherein the optionally substituted acyl is —C(═O)R″^{A1}, wherein R″^{A1} is an unsubstituted C₁₋₁₂-alkyl.

6. (canceled)

7. (canceled)

8. (canceled)

9. (canceled)

10. The compound of claim 1, wherein R^{1A} is

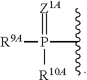

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. The compound of claim 10, wherein R^{9A} and R^{10A} are independently O⁻ or —OH.

32. (canceled)

33. The compound of claim 10, wherein R^{9A} is 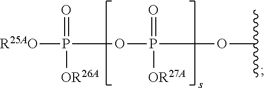
s is 0 or 1; R^{25A}, R^{26A} and R^{27A} are independently absent, hydrogen or deuterium; and R^{10A} is O⁻ or —OH.

34. The compound of claim 1, wherein R^{6A} is —OH.

35. The compound of claim 1, wherein R^{6A} is —OC(═O)R″^{A}.

36. The compound of claim 35, wherein R″^{A} is an unsubstituted C₁₋₁₂ alkyl.

37. (canceled)

38. (canceled)

39. (canceled)

40. The compound of claim 1, wherein R^{7A} is —OH.

41. (canceled)

42. (canceled)

43. The compound of claim 1, wherein R^{7A} is —OC(═O)R″^{B}, wherein R″^{B} is an unsubstituted C₁₋₁₂ alkyl.

44. (canceled)

45. (canceled)

46. (canceled)

47. The compound of claim 1, wherein R^{8A} is an optionally substituted C₂₋₆ alkynyl.

48. (canceled)

49. The compound of claim 47, wherein R^{8A} is an unsubstituted ethynyl.

50. (canceled)

51. The compound of claim 1, wherein B^{1A} is an optionally substituted

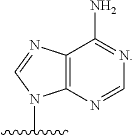 [SMILES: *n1cnc2c(N)ncnc21]

52. (canceled)

53. (canceled)

54. (canceled)

55. (canceled)

56. (canceled)

57. (canceled)

58. The compound of claim 51, wherein B^{1A} is an unsubstituted

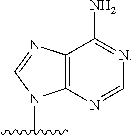 [SMILES: *n1cnc2c(N)ncnc21]

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. The compound of claim 1, selected from the group consisting of 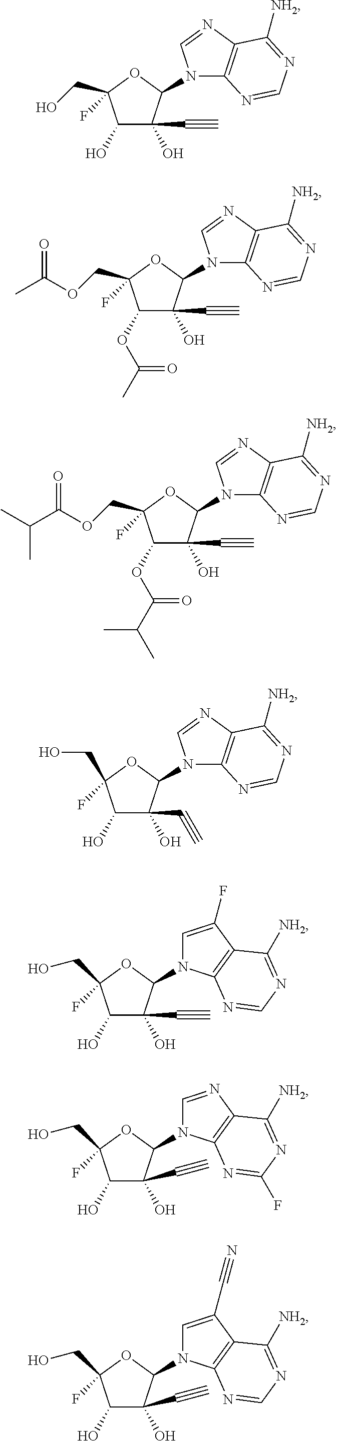 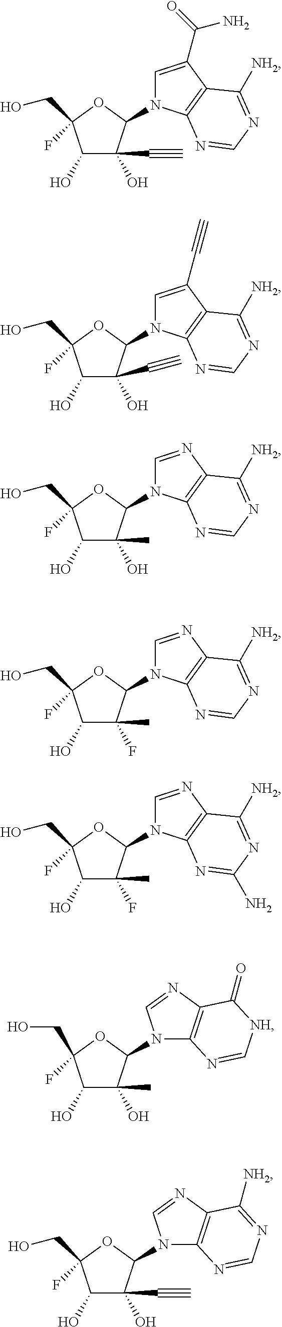 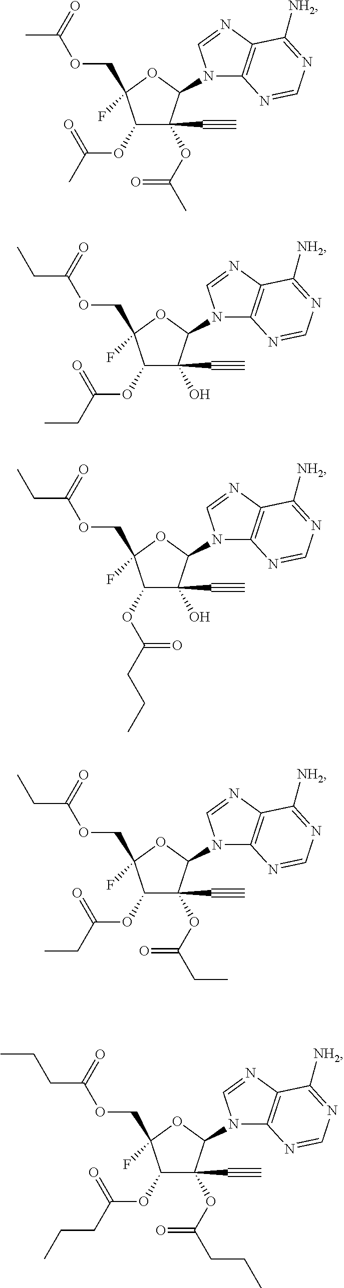 [SMILES: C#C[C@]1(OC(C)=O)[C@H](n2cnc3c(N)ncnc32)O[C@](F)(COC(C)=O)[C@H]1OC(C)=O ; C#C[C@]1(O)[C@H](n2cnc3c(N)ncnc32)O[C@](F)(COC(=O)CC)[C@H]1OC(=O)CC ; C#C[C@]1(O)[C@H](n2cnc3c(N)ncnc32)O[C@](F)(COC(=O)CC)[C@H]1OC(=O)CCC ; C#C[C@]1(OC(=O)CC)[C@H](n2cnc3c(N)ncnc32)O[C@](F)(COC(=O)CC)[C@H]1OC(=O)CC ; C#C[C@]1(OC(=O)CCC)[C@H](n2cnc3c(N)ncnc32)O[C@](F)(COC(=O)CCC)[C@H]1OC(=O)CCC] 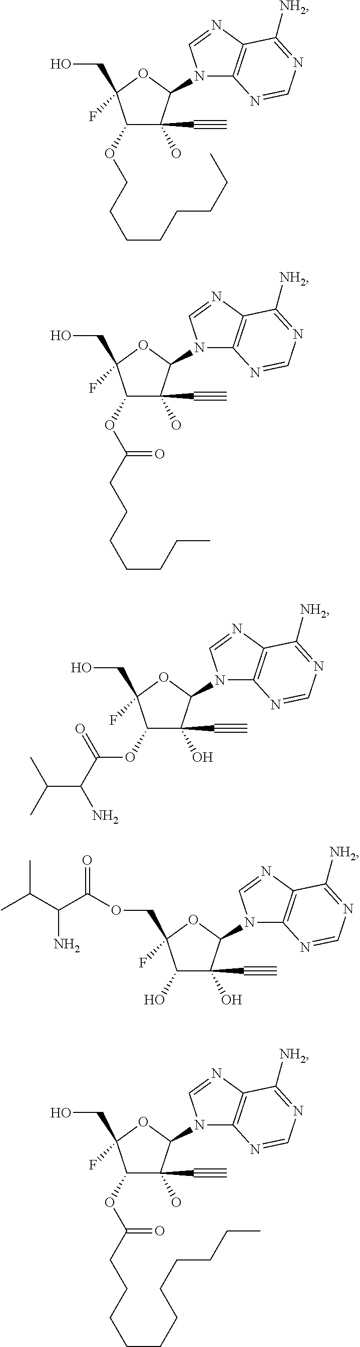 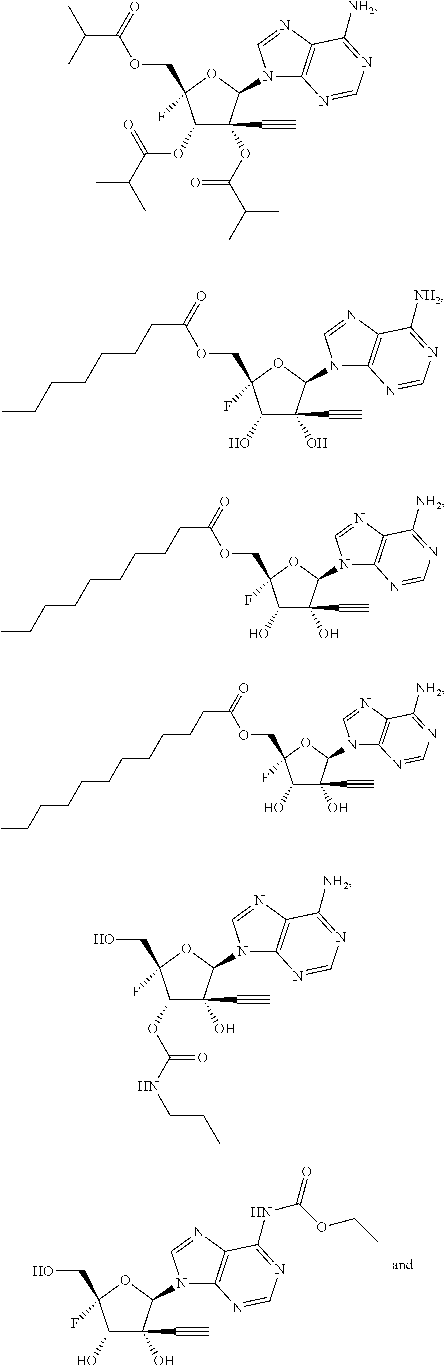 [SMILES: C#C[C@]1(OC(=O)C(C)C)[C@H](n2cnc3c(N)ncnc32)O[C@](F)(COC(=O)C(C)C)[C@H]1OC(=O)C(C)C ; C#C[C@]1(O)[C@H](n2cnc3c(N)ncnc32)O[C@](F)(COC(=O)CCCCCCC)[C@H]1O ; C#C[C@]1(O)[C@H](n2cnc3c(N)ncnc32)O[C@](F)(COC(=O)CCCCCCCCC)[C@H]1O ; C#C[C@]1(O)[C@H](n2cnc3c(N)ncnc32)O[C@](F)(COC(=O)CCCCCCCCCCC)[C@H]1O ; C#C[C@]1(O)[C@H](n2cnc3c(N)ncnc32)O[C@](F)(CO)[C@H]1OC(=O)NCCC ; C#C[C@]1(O)[C@H](n2cnc3c(NC(=O)OCC)ncnc32)O[C@](F)(CO)[C@H]1O] 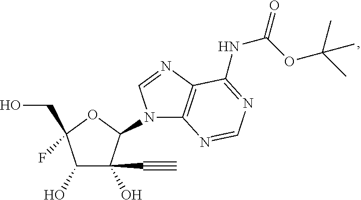 [SMILES: C#C[C@]1(O)[C@H](n2cnc3c(NC(=O)OC(C)(C)C)ncnc32)O[C@](F)(CO)[C@H]1O]
or a pharmaceutically acceptable salt of any of the foregoing.

82. The compound of claim 1, selected from the group consisting of 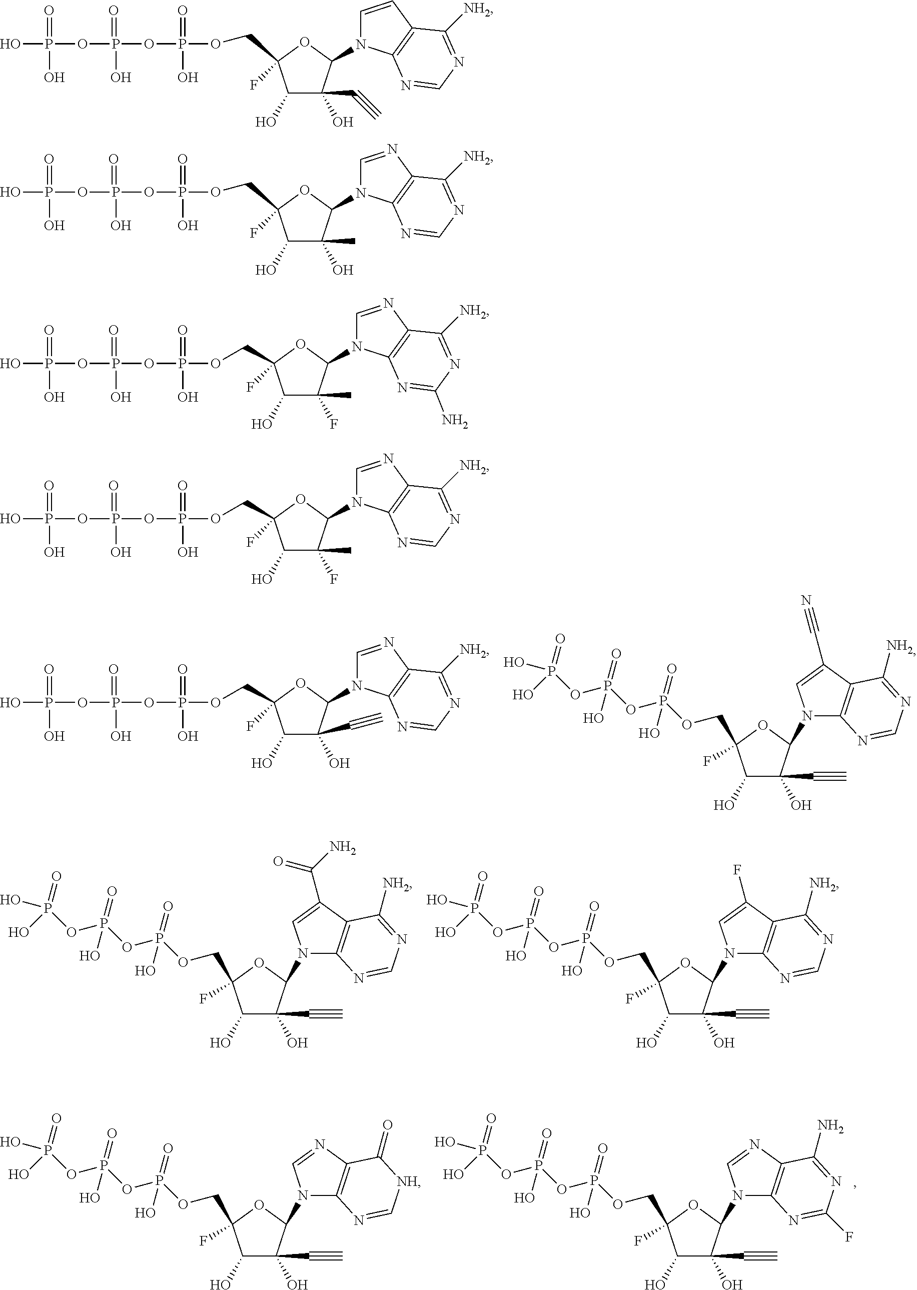 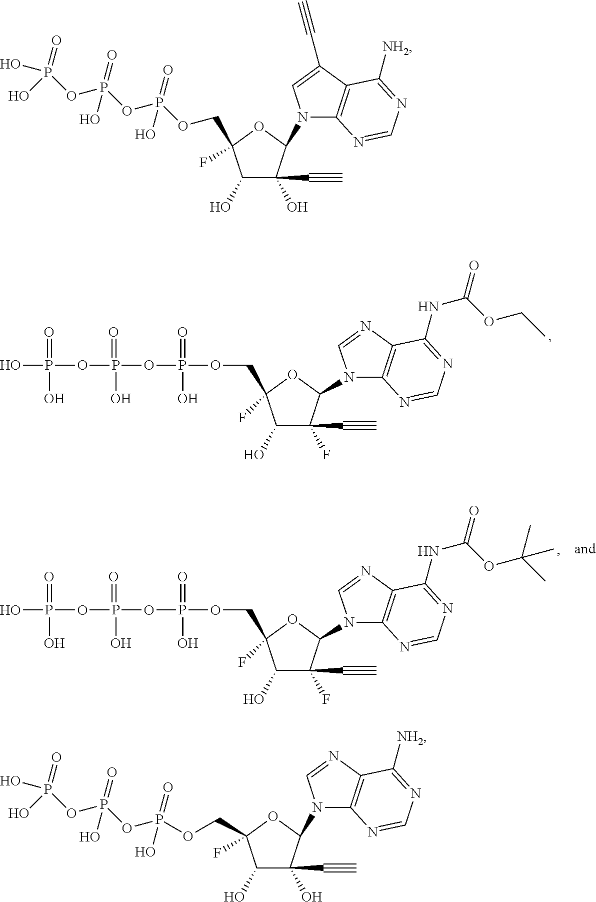 [SMILES: C#Cc1cn([C@@H]2O[C@](F)(COP(=O)(O)OP(=O)(O)OP(=O)(O)O)[C@@H](O)[C@]2(O)C#C)c2ncnc(N)c12 ; C#C[C@]1(F)[C@H](n2cnc3c(NC(=O)OCC)ncnc32)O[C@](F)(COP(=O)(O)OP(=O)(O)OP(=O)(O)O)[C@H]1O ; C#C[C@]1(F)[C@H](n2cnc3c(NC(=O)OC(C)(C)C)ncnc32)O[C@](F)(COP(=O)(O)OP(=O)(O)OP(=O)(O)O)[C@H]1O ; C#C[C@]1(O)[C@H](n2cnc3c(N)ncnc32)O[C@](F)(COP(=O)(O)OP(=O)(O)OP(=O)(O)O)[C@H]1O]
or a pharmaceutically acceptable salt of any of the foregoing.

83. (canceled)

84. (canceled)

85. (canceled)

86. (canceled)

87. (canceled)

88. (canceled)

89. (canceled)

90. (canceled)

91. (canceled)

92. (canceled)

93. (canceled)

94. (canceled)

95. (canceled)

96. (canceled)

97. (canceled)

98. (canceled)

99. A method of ameliorating and/or treating a Picornaviridae viral infection, comprising administering to a subject identified as suffering from the Picornaviridae viral infection an effective amount of one or more compounds of claim 1, or a pharmaceutically acceptable salt thereof.

100. A method of ameliorating and/or treating a Flaviviridae viral infection, comprising administering to a subject identified as suffering from the Flaviviridae viral infection an effective amount of one or more compounds of claim 1, or a pharmaceutically acceptable salt thereof.
